SEQUENCE LISTING (948 sequences)

SEQ ID NO: 1 - protein, 10 aa, Artificial Sequence
  Description of Artificial Sequence Synthetic peptide
GFTFSDYAMG

SEQ ID NO: 2 - protein, 17 aa, Artificial Sequence
  Description of Artificial Sequence Synthetic peptide
TISGSGGLTYYADSVKG

SEQ ID NO: 3 - protein, 9 aa, Artificial Sequence
  Description of Artificial Sequence Synthetic peptide
APYGYYMDV

SEQ ID NO: 4 - protein, 11 aa, Artificial Sequence
  Description of Artificial Sequence Synthetic peptide
RASQSISSWLA

SEQ ID NO: 5 - protein, 7 aa, Artificial Sequence
  Description of Artificial Sequence Synthetic peptide
KASSLES

SEQ ID NO: 6 - protein, 8 aa, Artificial Sequence
  Description of Artificial Sequence Synthetic peptide
QQYKSYIT

SEQ ID NO: 7 - protein, 5 aa, Artificial Sequence
  Description of Artificial Sequence Synthetic peptide
DYAMG

SEQ ID NO: 8 - protein, 17 aa, Artificial Sequence
  Description of Artificial Sequence Synthetic peptide
TISGSGGLTYYADSVKG

SEQ ID NO: 9 - protein, 9 aa, Artificial Sequence
  Description of Artificial Sequence Synthetic peptide
APYGYYMDV

SEQ ID NO: 10 - protein, 11 aa, Artificial Sequence
  Description of Artificial Sequence Synthetic peptide
RASQSISSWLA

SEQ ID NO: 11 - protein, 7 aa, Artificial Sequence
  Description of Artificial Sequence Synthetic peptide
KASSLES

SEQ ID NO: 12 - protein, 8 aa, Artificial Sequence
  Description of Artificial Sequence Synthetic peptide
QQYKSYIT

SEQ ID NO: 13 - protein, 7 aa, Artificial Sequence
  Description of Artificial Sequence Synthetic peptide
GFTFSDY

SEQ ID NO: 14 - protein, 4 aa, Artificial Sequence
  Description of Artificial Sequence Synthetic peptide
GSGG

SEQ ID NO: 15 - protein, 7 aa, Artificial Sequence
  Description of Artificial Sequence Synthetic peptide
PYGYYMD

SEQ ID NO: 16 - protein, 7 aa, Artificial Sequence
  Description of Artificial Sequence Synthetic peptide
SQSISSW

SEQ ID NO: 17 - protein, 3 aa, Artificial Sequence
  Description of Artificial Sequence Synthetic peptide
KAS

SEQ ID NO: 18 - protein, 5 aa, Artificial Sequence
  Description of Artificial Sequence Synthetic peptide
YKSYI

SEQ ID NO: 19 - protein, 10 aa, Artificial Sequence
  Description of Artificial Sequence Synthetic peptide
GFTFSDYAMG

SEQ ID NO: 20 - protein, 10 aa, Artificial Sequence
  Description of Artificial Sequence Synthetic peptide
TISGSGGLTY

SEQ ID NO: 21 - protein, 9 aa, Artificial Sequence
  Description of Artificial Sequence Synthetic peptide
APYGYYMDV

SEQ ID NO: 22 - protein, 11 aa, Artificial Sequence
  Description of Artificial Sequence Synthetic peptide
RASQSISSWLA

SEQ ID NO: 23 - protein, 7 aa, Artificial Sequence
  Description of Artificial Sequence Synthetic peptide
KASSLES

SEQ ID NO: 24 - protein, 8 aa, Artificial Sequence
  Description of Artificial Sequence Synthetic peptide
QQYKSYIT

SEQ ID NO: 25 - protein, 6 aa, Artificial Sequence
  Description of Artificial Sequence Synthetic peptide
SDYAMG

SEQ ID NO: 26 - protein, 13 aa, Artificial Sequence
  Description of Artificial Sequence Synthetic peptide
WVSTISGSGGLTY

SEQ ID NO: 27 - protein, 10 aa, Artificial Sequence
  Description of Artificial Sequence Synthetic peptide
AKAPYGYYMD

SEQ ID NO: 28 - protein, 7 aa, Artificial Sequence
  Description of Artificial Sequence Synthetic peptide
SSWLAWY

SEQ ID NO: 29 - protein, 10 aa, Artificial Sequence
  Description of Artificial Sequence Synthetic peptide
LLIYKASSLE

SEQ ID NO: 30 - protein, 7 aa, Artificial Sequence
  Description of Artificial Sequence Synthetic peptide
QQYKSYI

SEQ ID NO: 31 - protein, 8 aa, Artificial Sequence
  Description of Artificial Sequence Synthetic peptide
GFTFSDYA

SEQ ID NO: 32 - protein, 8 aa, Artificial Sequence
  Description of Artificial Sequence Synthetic peptide
ISGSGGLT

SEQ ID NO: 33 - protein, 11 aa, Artificial Sequence
  Description of Artificial Sequence Synthetic peptide
AKAPYGYYMDV

SEQ ID NO: 34 - protein, 6 aa, Artificial Sequence
  Description of Artificial Sequence Synthetic peptide
QSISSW

SEQ ID NO: 35 - protein, 3 aa, Artificial Sequence
  Description of Artificial Sequence Synthetic peptide
KAS

SEQ ID NO: 36 - protein, 8 aa, Artificial Sequence
  Description of Artificial Sequence Synthetic peptide
QQYKSYIT

SEQ ID NO: 37 - protein, 118 aa, Artificial Sequence
  Description of Artificial Sequence Synthetic polypeptide
EVQLLESGGGLVQPGGSLRLSCAASGFTFSDYAMGWVRQAPGKGLEWVSTISGSGGLTYYADSVKGRFTISRDNSKNTLYLQMNSLRAEDTAVYYCAKAPYGYYMDVWGKGTTVTVSS

SEQ ID NO: 38 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYKSYITFGGGTKVEIK

SEQ ID NO: 39 - protein, 10 aa, Artificial Sequence
  Description of Artificial Sequence Synthetic peptide
GFTFSSYAMA

SEQ ID NO: 40 - protein, 17 aa, Artificial Sequence
  Description of Artificial Sequence Synthetic peptide
AISGSGGLTYYADSVKG

SEQ ID NO: 41 - protein, 9 aa, Artificial Sequence
  Description of Artificial Sequence Synthetic peptide
APYGYYMDV

SEQ ID NO: 42 - protein, 11 aa, Artificial Sequence
  Description of Artificial Sequence Synthetic peptide
RASQSISSWLA

SEQ ID NO: 43 - protein, 7 aa, Artificial Sequence
  Description of Artificial Sequence Synthetic peptide
KASSLES

SEQ ID NO: 44 - protein, 8 aa, Artificial Sequence
  Description of Artificial Sequence Synthetic peptide
QQYKSYIT

SEQ ID NO: 45 - protein, 5 aa, Artificial Sequence
  Description of Artificial Sequence Synthetic peptide
SYAMA

SEQ ID NO: 46 - protein, 17 aa, Artificial Sequence
  Description of Artificial Sequence Synthetic peptide
AISGSGGLTYYADSVKG

SEQ ID NO: 47 - protein, 9 aa, Artificial Sequence
  Description of Artificial Sequence Synthetic peptide
APYGYYMDV

SEQ ID NO: 48 - protein, 11 aa, Artificial Sequence
  Description of Artificial Sequence Synthetic peptide
RASQSISSWLA

SEQ ID NO: 49 - protein, 7 aa, Artificial Sequence
  Description of Artificial Sequence Synthetic peptide
KASSLES

SEQ ID NO: 50 - protein, 8 aa, Artificial Sequence
  Description of Artificial Sequence Synthetic peptide
QQYKSYIT

SEQ ID NO: 51 - protein, 7 aa, Artificial Sequence
  Description of Artificial Sequence Synthetic peptide
GFTFSSY

SEQ ID NO: 52 - protein, 4 aa, Artificial Sequence
  Description of Artificial Sequence Synthetic peptide
GSGG

SEQ ID NO: 53 - protein, 7 aa, Artificial Sequence
  Description of Artificial Sequence Synthetic peptide
PYGYYMD

SEQ ID NO: 54 - protein, 7 aa, Artificial Sequence
  Description of Artificial Sequence Synthetic peptide
SQSISSW

SEQ ID NO: 55 - protein, 3 aa, Artificial Sequence
  Description of Artificial Sequence Synthetic peptide
KAS

SEQ ID NO: 56 - protein, 5 aa, Artificial Sequence
  Description of Artificial Sequence Synthetic peptide
YKSYI

SEQ ID NO: 57 - protein, 10 aa, Artificial Sequence
  Description of Artificial Sequence Synthetic peptide
GFTFSSYAMA

SEQ ID NO: 58 - protein, 10 aa, Artificial Sequence
  Description of Artificial Sequence Synthetic peptide
AISGSGGLTY

SEQ ID NO: 59 - protein, 9 aa, Artificial Sequence
  Description of Artificial Sequence Synthetic peptide
APYGYYMDV

SEQ ID NO: 60 - protein, 11 aa, Artificial Sequence
  Description of Artificial Sequence Synthetic peptide
RASQSISSWLA

SEQ ID NO: 61 - protein, 7 aa, Artificial Sequence
  Description of Artificial Sequence Synthetic peptide
KASSLES

SEQ ID NO: 62 - protein, 8 aa, Artificial Sequence
  Description of Artificial Sequence Synthetic peptide
QQYKSYIT

SEQ ID NO: 63 - protein, 6 aa, Artificial Sequence
  Description of Artificial Sequence Synthetic peptide
SSYAMA

SEQ ID NO: 64 - protein, 13 aa, Artificial Sequence
  Description of Artificial Sequence Synthetic peptide
WVSAISGSGGLTY

SEQ ID NO: 65 - protein, 10 aa, Artificial Sequence
  Description of Artificial Sequence Synthetic peptide
AKAPYGYYMD

SEQ ID NO: 66 - protein, 7 aa, Artificial Sequence
  Description of Artificial Sequence Synthetic peptide
SSWLAWY

SEQ ID NO: 67 - protein, 10 aa, Artificial Sequence
  Description of Artificial Sequence Synthetic peptide
LLIYKASSLE

SEQ ID NO: 68 - protein, 7 aa, Artificial Sequence
  Description of Artificial Sequence Synthetic peptide
QQYKSYI

SEQ ID NO: 69 - protein, 8 aa, Artificial Sequence
  Description of Artificial Sequence Synthetic peptide
GFTFSSYA

SEQ ID NO: 70 - protein, 8 aa, Artificial Sequence
  Description of Artificial Sequence Synthetic peptide
ISGSGGLT

SEQ ID NO: 71 - protein, 11 aa, Artificial Sequence
  Description of Artificial Sequence Synthetic peptide
AKAPYGYYMDV

SEQ ID NO: 72 - protein, 6 aa, Artificial Sequence
  Description of Artificial Sequence Synthetic peptide
QSISSW

SEQ ID NO: 73 - protein, 3 aa, Artificial Sequence
  Description of Artificial Sequence Synthetic peptide
KAS

SEQ ID NO: 74 - protein, 8 aa, Artificial Sequence
  Description of Artificial Sequence Synthetic peptide
QQYKSYIT

SEQ ID NO: 75 - protein, 118 aa, Artificial Sequence
  Description of Artificial Sequence Synthetic polypeptide
EVQLLESGGGLVQPGGSLRLSCAASGFTFSSYAMAWVRQAPGKGLEWVSAISGSGGLTYYADSVKGRFTISRDNSKNTLYLQMNSLRAEDTAVYYCAKAPYGYYMDVWGKGTTVTVSS

SEQ ID NO: 76 - protein, 106 aa, Artificial Sequence; identical to SEQ ID NO: 38
  Description of Artificial Sequence Synthetic polypeptide

SEQ ID NO: 77 - protein, 10 aa, Artificial Sequence
  Description of Artificial Sequence Synthetic peptide
GYTFDVYGIS

SEQ ID NO: 78 - protein, 17 aa, Artificial Sequence
  Description of Artificial Sequence Synthetic peptide
WIAPYNGNTNYAQKLQG

SEQ ID NO: 79 - protein, 14 aa, Artificial Sequence
  Description of Artificial Sequence Synthetic peptide
DAGTYSPFGYGMDV

SEQ ID NO: 80 - protein, 11 aa, Artificial Sequence
  Description of Artificial Sequence Synthetic peptide
RASQSISSWLA

SEQ ID NO: 81 - protein, 7 aa, Artificial Sequence
  Description of Artificial Sequence Synthetic peptide
KASSLES

SEQ ID NO: 82 - protein, 10 aa, Artificial Sequence
  Description of Artificial Sequence Synthetic peptide
QQFQSLPPFT

SEQ ID NO: 83 - protein, 5 aa, Artificial Sequence
  Description of Artificial Sequence Synthetic peptide
VYGIS

SEQ ID NO: 84 - protein, 17 aa, Artificial Sequence
  Description of Artificial Sequence Synthetic peptide
WIAPYNGNTNYAQKLQG

SEQ ID NO: 85 - protein, 14 aa, Artificial Sequence
  Description of Artificial Sequence Synthetic peptide
DAGTYSPFGYGMDV

SEQ ID NO: 86 - protein, 11 aa, Artificial Sequence
  Description of Artificial Sequence Synthetic peptide
RASQSISSWLA

SEQ ID NO: 87 - protein, 7 aa, Artificial Sequence
  Description of Artificial Sequence Synthetic peptide
KASSLES

SEQ ID NO: 88 - protein, 10 aa, Artificial Sequence
  Description of Artificial Sequence Synthetic peptide
QQFQSLPPFT

SEQ ID NO: 89 - protein, 7 aa, Artificial Sequence
  Description of Artificial Sequence Synthetic peptide
GYTFDVY

SEQ ID NO: 90 - protein, 4 aa, Artificial Sequence
  Description of Artificial Sequence Synthetic peptide
PYNG

SEQ ID NO: 91 - protein, 12 aa, Artificial Sequence
  Description of Artificial Sequence Synthetic peptide
AGTYSPFGYGMD

SEQ ID NO: 92 - protein, 7 aa, Artificial Sequence
  Description of Artificial Sequence Synthetic peptide
SQSISSW

SEQ ID NO: 93 - protein, 3 aa, Artificial Sequence
  Description of Artificial Sequence Synthetic peptide
KAS

SEQ ID NO: 94 - protein, 7 aa, Artificial Sequence
  Description of Artificial Sequence Synthetic peptide
FQSLPPF

SEQ ID NO: 95 - protein, 10 aa, Artificial Sequence
  Description of Artificial Sequence Synthetic peptide
GYTFDVYGIS

SEQ ID NO: 96 - protein, 10 aa, Artificial Sequence
  Description of Artificial Sequence Synthetic peptide
WIAPYNGNTN

SEQ ID NO: 97 - protein, 14 aa, Artificial Sequence
  Description of Artificial Sequence Synthetic peptide
DAGTYSPFGYGMDV

SEQ ID NO: 98 - protein, 11 aa, Artificial Sequence
  Description of Artificial Sequence Synthetic peptide
RASQSISSWLA

SEQ ID NO: 99 - protein, 7 aa, Artificial Sequence
  Description of Artificial Sequence Synthetic peptide
KASSLES

SEQ ID NO: 100 - protein, 10 aa, Artificial Sequence
  Description of Artificial Sequence Synthetic peptide
QQFQSLPPFT

SEQ ID NO: 101 - protein, 6 aa, Artificial Sequence
  Description of Artificial Sequence Synthetic peptide
DVYGIS

SEQ ID NO: 102 - protein, 13 aa, Artificial Sequence
  Description of Artificial Sequence Synthetic peptide
WMGWIAPYNGNTN

SEQ ID NO: 103 - protein, 15 aa, Artificial Sequence
  Description of Artificial Sequence Synthetic peptide
ARDAGTYSPFGYGMD

SEQ ID NO: 104 - protein, 7 aa, Artificial Sequence
  Description of Artificial Sequence Synthetic peptide
SSWLAWY

SEQ ID NO: 105 - protein, 10 aa, Artificial Sequence
  Description of Artificial Sequence Synthetic peptide
LLIYKASSLE

SEQ ID NO: 106 - protein, 9 aa, Artificial Sequence
  Description of Artificial Sequence Synthetic peptide
QQFQSLPPF

SEQ ID NO: 107 - protein, 8 aa, Artificial Sequence
  Description of Artificial Sequence Synthetic peptide
GYTFDVYG

SEQ ID NO: 108 - protein, 8 aa, Artificial Sequence
  Description of Artificial Sequence Synthetic peptide
IAPYNGNT

SEQ ID NO: 109 - protein, 16 aa, Artificial Sequence
  Description of Artificial Sequence Synthetic peptide
ARDAGTYSPFGTGMDV

SEQ ID NO: 110 - protein, 6 aa, Artificial Sequence
  Description of Artificial Sequence Synthetic peptide
QSISSW

SEQ ID NO: 111 - protein, 3 aa, Artificial Sequence
  Description of Artificial Sequence Synthetic peptide
KAS

SEQ ID NO: 112 - protein, 10 aa, Artificial Sequence
  Description of Artificial Sequence Synthetic peptide
QQFQSLPPFT

SEQ ID NO: 113 - protein, 123 aa, Artificial Sequence
  Description of Artificial Sequence Synthetic polypeptide
QVQLVQSGAEVKKPGASVKVSCKASGYTFDVYGISWVRQAPGQGLEWMGWIAPYNGNTNYAQKLQGRVTMTTDTSTSTAYMELRSLRSDDTAVYYCARDAGTYSPFGYGMDVWGQGTTVTVSS

SEQ ID NO: 114 - protein, 108 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQFQSLPPFTFGGGTKVEIK

SEQ ID NO: 115 - protein, 10 aa, Artificial Sequence
  Description of Artificial Sequence Synthetic peptide
GYTFDVYGIS

SEQ ID NO: 116 - protein, 17 aa, Artificial Sequence
  Description of Artificial Sequence Synthetic peptide
WIAPYSGNTNYAQKLQG

SEQ ID NO: 117 - protein, 14 aa, Artificial Sequence
  Description of Artificial Sequence Synthetic peptide
DAGTYSPFGYGMDV

SEQ ID NO: 118 - protein, 11 aa, Artificial Sequence
  Description of Artificial Sequence Synthetic peptide
QASQSINNWLA

SEQ ID NO: 119 - protein, 7 aa, Artificial Sequence
  Description of Artificial Sequence Synthetic peptide
KAYNLES

SEQ ID NO: 120 - protein, 10 aa, Artificial Sequence
  Description of Artificial Sequence Synthetic peptide
QLFQSLPPFT

SEQ ID NO: 121 - protein, 5 aa, Artificial Sequence
  Description of Artificial Sequence Synthetic peptide
VYGIS

SEQ ID NO: 122 - protein, 17 aa, Artificial Sequence
  Description of Artificial Sequence Synthetic peptide
WIAPYSGNTNYAQKLQG

SEQ ID NO: 123 - protein, 14 aa, Artificial Sequence
  Description of Artificial Sequence Synthetic peptide
DAGTYSPFGYGMDV

SEQ ID NO: 124 - protein, 11 aa, Artificial Sequence
  Description of Artificial Sequence Synthetic peptide
QASQSINNWLA

SEQ ID NO: 125 - protein, 7 aa, Artificial Sequence
  Description of Artificial Sequence Synthetic peptide
KAYNLES

SEQ ID NO: 126 - protein, 10 aa, Artificial Sequence
  Description of Artificial Sequence Synthetic peptide
QLFQSLPPFT

SEQ ID NO: 127 - protein, 7 aa, Artificial Sequence
  Description of Artificial Sequence Synthetic peptide
GYTFDVY

SEQ ID NO: 128 - protein, 4 aa, Artificial Sequence
  Description of Artificial Sequence Synthetic peptide
PYSG

SEQ ID NO: 129 - protein, 12 aa, Artificial Sequence
  Description of Artificial Sequence Synthetic peptide
AGTYSPFGYGMD

SEQ ID NO: 130 - protein, 7 aa, Artificial Sequence
  Description of Artificial Sequence Synthetic peptide
SQSINNW

SEQ ID NO: 131 - protein, 3 aa, Artificial Sequence
  Description of Artificial Sequence Synthetic peptide
KAY

SEQ ID NO: 132 - protein, 7 aa, Artificial Sequence
  Description of Artificial Sequence Synthetic peptide
FQSLPPF

SEQ ID NO: 133 - protein, 10 aa, Artificial Sequence
  Description of Artificial Sequence Synthetic peptide
GYTFDVYGIS

SEQ ID NO: 134 - protein, 10 aa, Artificial Sequence
  Description of Artificial Sequence Synthetic peptide
WIAPYSGNTN

SEQ ID NO: 135 - protein, 14 aa, Artificial Sequence
  Description of Artificial Sequence Synthetic peptide
DAGTYSPFGYGMDV

SEQ ID NO: 136 - protein, 11 aa, Artificial Sequence
  Description of Artificial Sequence Synthetic peptide
QASQSINNWLA

SEQ ID NO: 137 - protein, 7 aa, Artificial Sequence
  Description of Artificial Sequence Synthetic peptide
KAYNLES

SEQ ID NO: 138 - protein, 10 aa, Artificial Sequence
  Description of Artificial Sequence Synthetic peptide
QLFQSLPPFT

SEQ ID NO: 139 - protein, 6 aa, Artificial Sequence
  Description of Artificial Sequence Synthetic peptide
DVYGIS

SEQ ID NO: 140 - protein, 13 aa, Artificial Sequence
  Description of Artificial Sequence Synthetic peptide
WMGWIAPYSGNTN

SEQ ID NO: 141 - protein, 15 aa, Artificial Sequence
  Description of Artificial Sequence Synthetic peptide
ARDAGTYSPFGYGMD

SEQ ID NO: 142 - protein, 7 aa, Artificial Sequence
  Description of Artificial Sequence Synthetic peptide
NNWLAWY

SEQ ID NO: 143 - protein, 10 aa, Artificial Sequence
  Description of Artificial Sequence Synthetic peptide
LLIYKAYNLE

SEQ ID NO: 144 - protein, 9 aa, Artificial Sequence
  Description of Artificial Sequence Synthetic peptide
QLFQSLPPF

SEQ ID NO: 145 - protein, 8 aa, Artificial Sequence
  Description of Artificial Sequence Synthetic peptide
GYTFDVYG

SEQ ID NO: 146 - protein, 8 aa, Artificial Sequence
  Description of Artificial Sequence Synthetic peptide
IAPYSGNT

SEQ ID NO: 147 - protein, 16 aa, Artificial Sequence
  Description of Artificial Sequence Synthetic peptide
ARDAGTYSPFGTGMDV

SEQ ID NO: 148 - protein, 6 aa, Artificial Sequence
  Description of Artificial Sequence Synthetic peptide
QSINNW

SEQ ID NO: 149 - protein, 3 aa, Artificial Sequence
  Description of Artificial Sequence Synthetic peptide
KAY

SEQ ID NO: 150 - protein, 10 aa, Artificial Sequence
  Description of Artificial Sequence Synthetic peptide
QLFQSLPPFT

SEQ ID NO: 151 - protein, 123 aa, Artificial Sequence
  Description of Artificial Sequence Synthetic polypeptide
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSS

SEQ ID NO: 152 - protein, 108 aa, Artificial Sequence
  Description of Artificial Sequence Synthetic polypeptide
DIQMTQSPSTLSASVGDRVTITCQASQSINNWLAWYQQKPGKAPKLLIYKAYNLESGVPSRFSGSGSGTEFTLTISSLQPDDFATYYCQLFQSLPPFTFGGGTKVEIK

SEQ ID NO: 153 - protein, 10 aa, Artificial Sequence
  Description of Artificial Sequence Synthetic peptide
GYTFDVYGIS

SEQ ID NO: 154 - protein, 17 aa, Artificial Sequence
  Description of Artificial Sequence Synthetic peptide
WIAPYSGNTNYAQKLQG

SEQ ID NO: 155 - protein, 14 aa, Artificial Sequence
  Description of Artificial Sequence Synthetic peptide
DAGTYSPFGYGMDV

SEQ ID NO: 156 - protein, 11 aa, Artificial Sequence
  Description of Artificial Sequence Synthetic peptide
RASESISNWLA

SEQ ID NO: 157 - protein, 7 aa, Artificial Sequence
  Description of Artificial Sequence Synthetic peptide
KAYSLEY

SEQ ID NO: 158 - protein, 10 aa, Artificial Sequence
  Description of Artificial Sequence Synthetic peptide
QQFQKLPPFT

SEQ ID NO: 159 - protein, 5 aa, Artificial Sequence
  Description of Artificial Sequence Synthetic peptide
VYGIS

SEQ ID NO: 160 - protein, 17 aa, Artificial Sequence
  Description of Artificial Sequence Synthetic peptide
WIAPYSGNTNYAQKLQG

SEQ ID NO: 161 - protein, 14 aa, Artificial Sequence
  Description of Artificial Sequence Synthetic peptide
DAGTYSPFGYGMDV

SEQ ID NO: 162 - protein, 11 aa, Artificial Sequence
  Description of Artificial Sequence Synthetic peptide
RASESISNWLA

SEQ ID NO: 163 - protein, 7 aa, Artificial Sequence
  Description of Artificial Sequence Synthetic peptide
KAYSLEY

SEQ ID NO: 164 - protein, 10 aa, Artificial Sequence
  Description of Artificial Sequence Synthetic peptide
QQFQKLPPFT

SEQ ID NO: 165 - protein, 7 aa, Artificial Sequence
  Description of Artificial Sequence Synthetic peptide
GYTFDVY

SEQ ID NO: 166 - protein, 4 aa, Artificial Sequence
  Description of Artificial Sequence Synthetic peptide
PYSG

SEQ ID NO: 167 - protein, 12 aa, Artificial Sequence
  Description of Artificial Sequence Synthetic peptide
AGTYSPFGYGMD

SEQ ID NO: 168 - protein, 7 aa, Artificial Sequence
  Description of Artificial Sequence Synthetic peptide
SESISNW

SEQ ID NO: 169 - protein, 3 aa, Artificial Sequence
  Description of Artificial Sequence Synthetic peptide
KAY

SEQ ID NO: 170 - protein, 7 aa, Artificial Sequence
  Description of Artificial Sequence Synthetic peptide
FQKLPPF

SEQ ID NO: 171 - protein, 10 aa, Artificial Sequence
  Description of Artificial Sequence Synthetic peptide
GYTFDVYGIS

SEQ ID NO: 172 - protein, 10 aa, Artificial Sequence
  Description of Artificial Sequence Synthetic peptide
WIAPYSGNTN

SEQ ID NO: 173 - protein, 14 aa, Artificial Sequence
  Description of Artificial Sequence Synthetic peptide
DAGTYSPFGYGMDV

SEQ ID NO: 174 - protein, 11 aa, Artificial Sequence
  Description of Artificial Sequence Synthetic peptide
RASESISNWLA

SEQ ID NO: 175 - protein, 7 aa, Artificial Sequence
  Description of Artificial Sequence Synthetic peptide
KAYSLEY

SEQ ID NO: 176 - protein, 10 aa, Artificial Sequence
  Description of Artificial Sequence Synthetic peptide
QQFQKLPPFT

SEQ ID NO: 177 - protein, 6 aa, Artificial Sequence
  Description of Artificial Sequence Synthetic peptide
DVYGIS

SEQ ID NO: 178 - protein, 13 aa, Artificial Sequence
  Description of Artificial Sequence Synthetic peptide
WMGWIAPYSGNTN

SEQ ID NO: 179 - protein, 15 aa, Artificial Sequence
  Description of Artificial Sequence Synthetic peptide
ARDAGTYSPFGYGMD

SEQ ID NO: 180 - protein, 7 aa, Artificial Sequence
  Description of Artificial Sequence Synthetic peptide
SNWLAWY

SEQ ID NO: 181 - protein, 10 aa, Artificial Sequence
  Description of Artificial Sequence Synthetic peptide
LLIYKAYSLE

SEQ ID NO: 182 - protein, 9 aa, Artificial Sequence
  Description of Artificial Sequence Synthetic peptide
QQFQKLPPF

SEQ ID NO: 183 - protein, 8 aa, Artificial Sequence
  Description of Artificial Sequence Synthetic peptide
GYTFDVYG

SEQ ID NO: 184 - protein, 8 aa, Artificial Sequence
  Description of Artificial Sequence Synthetic peptide
IAPYSGNT

SEQ ID NO: 185 - protein, 16 aa, Artificial Sequence
  Description of Artificial Sequence Synthetic peptide
ARDAGTYSPFGTGMDV

SEQ ID NO: 186 - protein, 6 aa, Artificial Sequence
  Description of Artificial Sequence Synthetic peptide
ESISNW

SEQ ID NO: 187 - protein, 3 aa, Artificial Sequence
  Description of Artificial Sequence Synthetic peptide
KAY

SEQ ID NO: 188 - protein, 10 aa, Artificial Sequence
  Description of Artificial Sequence Synthetic peptide
QQFQKLPPFT

SEQ ID NO: 189 - protein, 123 aa, Artificial Sequence; identical to SEQ ID NO: 151
  Description of Artificial Sequence Synthetic polypeptide

SEQ ID NO: 190 - protein, 108 aa, Artificial Sequence
  Description of Artificial Sequence Synthetic polypeptide
DIQMTQSPSTLSASVGDRVTITCRASESISNWLAWYQQKPGKAPKLLIYKAYSLEYGVPSRFSGSGSGTEFTLTISSLQPDDFATYYCQQFQKLPPFTFGGGTKVEIK

SEQ ID NO: 191 - protein, 10 aa, Artificial Sequence
  Description of Artificial Sequence Synthetic peptide
GYTFRSYGIS

SEQ ID NO: 192 - protein, 17 aa, Artificial Sequence
  Description of Artificial Sequence Synthetic peptide
WVAPYNGNTNYAQKLQG

SEQ ID NO: 193 - protein, 14 aa, Artificial Sequence
  Description of Artificial Sequence Synthetic peptide
DAGTYSPYGYGMDV

SEQ ID NO: 194 - protein, 11 aa, Artificial Sequence
  Description of Artificial Sequence Synthetic peptide
RASQSISSWLA

SEQ ID NO: 195 - protein, 7 aa, Artificial Sequence
  Description of Artificial Sequence Synthetic peptide
KASSLES

SEQ ID NO: 196 - protein, 10 aa, Artificial Sequence
  Description of Artificial Sequence Synthetic peptide
QQFQSLPPFT

SEQ ID NO: 197 - protein, 5 aa, Artificial Sequence
  Description of Artificial Sequence Synthetic peptide
SYGIS

SEQ ID NO: 198 - protein, 17 aa, Artificial Sequence
  Description of Artificial Sequence Synthetic peptide
WVAPYNGNTNYAQKLQG

SEQ ID NO: 199 - protein, 14 aa, Artificial Sequence
  Description of Artificial Sequence Synthetic peptide
DAGTYSPYGYGMDV

SEQ ID NO: 200 - protein, 11 aa, Artificial Sequence
  Description of Artificial Sequence Synthetic peptide
RASQSISSWLA

SEQ ID NO: 201 - protein, 7 aa, Artificial Sequence
  Description of Artificial Sequence Synthetic peptide
KASSLES

SEQ ID NO: 202 - protein, 10 aa, Artificial Sequence
  Description of Artificial Sequence Synthetic peptide
QQFQSLPPFT

SEQ ID NO: 203 - protein, 7 aa, Artificial Sequence
  Description of Artificial Sequence Synthetic peptide
GYTFRSY

SEQ ID NO: 204 - protein, 4 aa, Artificial Sequence
  Description of Artificial Sequence Synthetic peptide
PYNG

SEQ ID NO: 205 - protein, 12 aa, Artificial Sequence
  Description of Artificial Sequence Synthetic peptide
AGTYSPYGYGMD

SEQ ID NO: 206 - protein, 7 aa, Artificial Sequence
  Description of Artificial Sequence Synthetic peptide
SQSISSW

SEQ ID NO: 207 - protein, 3 aa, Artificial Sequence
  Description of Artificial Sequence Synthetic peptide
KAS

SEQ ID NO: 208 - protein, 7 aa, Artificial Sequence
  Description of Artificial Sequence Synthetic peptide
FQSLPPF

SEQ ID NO: 209 - protein, 10 aa, Artificial Sequence
  Description of Artificial Sequence Synthetic peptide
GYTFRSYGIS

SEQ ID NO: 210 - protein, 10 aa, Artificial Sequence
  Description of Artificial Sequence Synthetic peptide
WVAPYNGNTN

SEQ ID NO: 211 - protein, 14 aa, Artificial Sequence
  Description of Artificial Sequence Synthetic peptide
DAGTYSPYGYGMDV

SEQ ID NO: 212 - protein, 11 aa, Artificial Sequence
  Description of Artificial Sequence Synthetic peptide
RASQSISSWLA

SEQ ID NO: 213 - protein, 7 aa, Artificial Sequence
  Description of Artificial Sequence Synthetic peptide
KASSLES

SEQ ID NO: 214 - protein, 10 aa, Artificial Sequence
  Description of Artificial Sequence Synthetic peptide
QQFQSLPPFT

SEQ ID NO: 215 - protein, 6 aa, Artificial Sequence
  Description of Artificial Sequence Synthetic peptide
RSYGIS

SEQ ID NO: 216 - protein, 13 aa, Artificial Sequence
  Description of Artificial Sequence Synthetic peptide
WMGWVAPYNGNTN

SEQ ID NO: 217 - protein, 15 aa, Artificial Sequence
  Description of Artificial Sequence Synthetic peptide
ARDAGTYSPYGYGMD

SEQ ID NO: 218 - protein, 7 aa, Artificial Sequence
  Description of Artificial Sequence Synthetic peptide
SSWLAWY

SEQ ID NO: 219 - protein, 10 aa, Artificial Sequence
  Description of Artificial Sequence Synthetic peptide
LLIYKASSLE

SEQ ID NO: 220 - protein, 9 aa, Artificial Sequence
  Description of Artificial Sequence Synthetic peptide
QQFQSLPPF

SEQ ID NO: 221 - protein, 8 aa, Artificial Sequence
  Description of Artificial Sequence Synthetic peptide
GYTFRSYG

SEQ ID NO: 222 - protein, 8 aa, Artificial Sequence
  Description of Artificial Sequence Synthetic peptide
VAPYNGNT

SEQ ID NO: 223 - protein, 16 aa, Artificial Sequence
  Description of Artificial Sequence Synthetic peptide
ARDAGTYSPYGYGMDV

SEQ ID NO: 224 - protein, 6 aa, Artificial Sequence
  Description of Artificial Sequence Synthetic peptide
QSISSW

SEQ ID NO: 225 - protein, 3 aa, Artificial Sequence
  Description of Artificial Sequence Synthetic peptide
KAS

SEQ ID NO: 226 - protein, 10 aa, Artificial Sequence
  Description of Artificial Sequence Synthetic peptide
QQFQSLPPFT

SEQ ID NO: 227 - protein, 123 aa, Artificial Sequence
  Description of Artificial Sequence Synthetic polypeptide
QVQLVQSGAEVKKPGASVKVSCKASGYTFRSYGISWVRQAPGQGLEWMGWVAPYNGNTNYAQKLQGRVTMTTDTSTSTAYMELRSLRSDDTAVYYCARDAGTYSPYGYGMDVWGQGTTVTVSS

SEQ ID NO: 228 - protein, 108 aa, Artificial Sequence; identical to SEQ ID NO: 114
  Description of Artificial Sequence Synthetic polypeptide

SEQ ID NO: 229 - protein, 10 aa, Artificial Sequence
  Description of Artificial Sequence Synthetic peptide
GYTFRSYGIS

SEQ ID NO: 230 - protein, 17 aa, Artificial Sequence
  Description of Artificial Sequence Synthetic peptide
WVAPYSGNTNYAQKLQG

SEQ ID NO: 231 - protein, 14 aa, Artificial Sequence
  Description of Artificial Sequence Synthetic peptide
DAGTYSPYGYGMDV

SEQ ID NO: 232 - protein, 11 aa, Artificial Sequence
  Description of Artificial Sequence Synthetic peptide
RASHSIDSWLA

SEQ ID NO: 233 - protein, 7 aa, Artificial Sequence
  Description of Artificial Sequence Synthetic peptide
KASYLES

SEQ ID NO: 234 - protein, 10 aa, Artificial Sequence
  Description of Artificial Sequence Synthetic peptide
QLFQSLPPFT

SEQ ID NO: 235 - protein, 5 aa, Artificial Sequence
  Description of Artificial Sequence Synthetic peptide
SYGIS

SEQ ID NO: 236 - protein, 17 aa, Artificial Sequence
  Description of Artificial Sequence Synthetic peptide
WVAPYSGNTNYAQKLQG

SEQ ID NO: 237 - protein, 14 aa, Artificial Sequence
  Description of Artificial Sequence Synthetic peptide
DAGTYSPYGYGMDV

SEQ ID NO: 238 - protein, 11 aa, Artificial Sequence
  Description of Artificial Sequence Synthetic peptide
RASHSIDSWLA

SEQ ID NO: 239 - protein, 7 aa, Artificial Sequence
  Description of Artificial Sequence Synthetic peptide
KASYLES

SEQ ID NO: 240 - protein, 10 aa, Artificial Sequence
  Description of Artificial Sequence Synthetic peptide
QLFQSLPPFT

SEQ ID NO: 241 - protein, 7 aa, Artificial Sequence
  Description of Artificial Sequence Synthetic peptide
GYTFRSY

SEQ ID NO: 242 - protein, 4 aa, Artificial Sequence
  Description of Artificial Sequence Synthetic peptide
PYSG

SEQ ID NO: 243 - protein, 12 aa, Artificial Sequence
  Description of Artificial Sequence Synthetic peptide
AGTYSPYGYGMD

SEQ ID NO: 244 - protein, 7 aa, Artificial Sequence
  Description of Artificial Sequence Synthetic peptide
SHSIDSW

SEQ ID NO: 245 - protein, 3 aa, Artificial Sequence
  Description of Artificial Sequence Synthetic peptide
KAS

SEQ ID NO: 246 - protein, 7 aa, Artificial Sequence
  Description of Artificial Sequence Synthetic peptide
FQSLPPF

SEQ ID NO: 247 - protein, 10 aa, Artificial Sequence
  Description of Artificial Sequence Synthetic peptide
GYTFRSYGIS

SEQ ID NO: 248 - protein, 10 aa, Artificial Sequence
  Description of Artificial Sequence Synthetic peptide
WVAPYSGNTN

SEQ ID NO: 249 - protein, 14 aa, Artificial Sequence
  Description of Artificial Sequence Synthetic peptide
DAGTYSPYGYGMDV

SEQ ID NO: 250 - protein, 11 aa, Artificial Sequence
  Description of Artificial Sequence Synthetic peptide
RASHSIDSWLA

SEQ ID NO: 251 - protein, 7 aa, Artificial Sequence
  Description of Artificial Sequence Synthetic peptide
KASYLES

SEQ ID NO: 252 - protein, 10 aa, Artificial Sequence
  Description of Artificial Sequence Synthetic peptide
QLFQSLPPFT

SEQ ID NO: 253 - protein, 6 aa, Artificial Sequence
  Description of Artificial Sequence Synthetic peptide
RSYGIS

SEQ ID NO: 254 - protein, 13 aa, Artificial Sequence
  Description of Artificial Sequence Synthetic peptide
WMGWVAPYSGNTN

SEQ ID NO: 255 - protein, 15 aa, Artificial Sequence
  Description of Artificial Sequence Synthetic peptide
ARDAGTYSPYGYGMD

SEQ ID NO: 256 - protein, 7 aa, Artificial Sequence
  Description of Artificial Sequence Synthetic peptide
DSWLAWY

SEQ ID NO: 257 - protein, 10 aa, Artificial Sequence
  Description of Artificial Sequence Synthetic peptide
LLIYKASYLE

SEQ ID NO: 258 - protein, 9 aa, Artificial Sequence
  Description of Artificial Sequence Synthetic peptide
QLFQSLPPF

SEQ ID NO: 259 - protein, 8 aa, Artificial Sequence
  Description of Artificial Sequence Synthetic peptide
GYTFRSYG

SEQ ID NO: 260 - protein, 8 aa, Artificial Sequence
  Description of Artificial Sequence Synthetic peptide
VAPYSGNT

SEQ ID NO: 261 - protein, 16 aa, Artificial Sequence
  Description of Artificial Sequence Synthetic peptide
ARDAGTYSPYGYGMDV

SEQ ID NO: 262 - protein, 6 aa, Artificial Sequence
  Description of Artificial Sequence Synthetic peptide
HSIDSW

SEQ ID NO: 263 - protein, 3 aa, Artificial Sequence
  Description of Artificial Sequence Synthetic peptide
KAS

SEQ ID NO: 264 - protein, 10 aa, Artificial Sequence
  Description of Artificial Sequence Synthetic peptide
QLFQSLPPFT

SEQ ID NO: 265 - protein, 123 aa, Artificial Sequence
  Description of Artificial Sequence Synthetic polypeptide
QVQLVQSGAEVKKPGASVKVSCKASGYTFRSYGISWVRQAPGQGLEWMGWVAPYSGNTNYAQKLQGRVTMTTDTSTSTAYMELRSLRSDDTAVYYCARDAGTYSPYGYGMDVWGQGTTVTVSS

SEQ ID NO: 266 - protein, 108 aa, Artificial Sequence
  Description of Artificial Sequence Synthetic polypeptide
DIQMTQSPSTLSASVGDRVTITCRASHSIDSWLAWYQQKPGKAPKLLIYKASYLESGVPSRFSGSGSGTEFTLTISSLQPDDFATYYCQLFQSLPPFTFGGGTKVEIK

SEQ ID NO: 267 - protein, 10 aa, Artificial Sequence
  Description of Artificial Sequence Synthetic peptide
GYTFRSYGIS

SEQ ID NO: 268 - protein, 17 aa, Artificial Sequence
  Description of Artificial Sequence Synthetic peptide
WVAPYSGNTNYAQKLQG

SEQ ID NO: 269 - protein, 14 aa, Artificial Sequence
  Description of Artificial Sequence Synthetic peptide
DAGTYSPYGYGMDV

SEQ ID NO: 270 - protein, 11 aa, Artificial Sequence
  Description of Artificial Sequence Synthetic peptide
QASQSIDSWLA

SEQ ID NO: 271 - protein, 7 aa, Artificial Sequence
  Description of Artificial Sequence Synthetic peptide
SASYLES

SEQ ID NO: 272 - protein, 10 aa, Artificial Sequence
  Description of Artificial Sequence Synthetic peptide
QRFQSLPPFT

SEQ ID NO: 273 - protein, 5 aa, Artificial Sequence
  Description of Artificial Sequence Synthetic peptide
SYGIS

SEQ ID NO: 274 - protein, 17 aa, Artificial Sequence
  Description of Artificial Sequence Synthetic peptide
WVAPYSGNTNYAQKLQG

SEQ ID NO: 275 - protein, 14 aa, Artificial Sequence
  Description of Artificial Sequence Synthetic peptide
DAGTYSPYGYGMDV

SEQ ID NO: 276 - protein, 11 aa, Artificial Sequence
  Description of Artificial Sequence Synthetic peptide
QASQSIDSWLA

SEQ ID NO: 277 - protein, 7 aa, Artificial Sequence
  Description of Artificial Sequence Synthetic peptide
SASYLES

SEQ ID NO: 278 - protein, 10 aa, Artificial Sequence
  Description of Artificial Sequence Synthetic peptide
QRFQSLPPFT

SEQ ID NO: 279 - protein, 7 aa, Artificial Sequence
  Description of Artificial Sequence Synthetic peptide
GYTFRSY

SEQ ID NO: 280 - protein, 4 aa, Artificial Sequence
  Description of Artificial Sequence Synthetic peptide
PYSG

SEQ ID NO: 281 - protein, 12 aa, Artificial Sequence
  Description of Artificial Sequence Synthetic peptide
AGTYSPYGYGMD

SEQ ID NO: 282 - protein, 7 aa, Artificial Sequence
  Description of Artificial Sequence Synthetic peptide
SQSIDSW

SEQ ID NO: 283 - protein, 3 aa, Artificial Sequence
  Description of Artificial Sequence Synthetic peptide
SAS

SEQ ID NO: 284 - protein, 7 aa, Artificial Sequence
  Description of Artificial Sequence Synthetic peptide
FQSLPPF

SEQ ID NO: 285 - protein, 10 aa, Artificial Sequence
  Description of Artificial Sequence Synthetic peptide
GYTFRSYGIS

SEQ ID NO: 286 - protein, 10 aa, Artificial Sequence
  Description of Artificial Sequence Synthetic peptide
WVAPYSGNTN

SEQ ID NO: 287 - protein, 14 aa, Artificial Sequence
  Description of Artificial Sequence Synthetic peptide
DAGTYSPYGYGMDV

SEQ ID NO: 288 - protein, 11 aa, Artificial Sequence
  Description of Artificial Sequence Synthetic peptide
QASQSIDSWLA

SEQ ID NO: 289 - protein, 7 aa, Artificial Sequence
  Description of Artificial Sequence Synthetic peptide
SASYLES

SEQ ID NO: 290 - protein, 10 aa, Artificial Sequence
  Description of Artificial Sequence Synthetic peptide
QRFQSLPPFT

SEQ ID NO: 291 - protein, 6 aa, Artificial Sequence
  Description of Artificial Sequence Synthetic peptide
RSYGIS

SEQ ID NO: 292 - protein, 13 aa, Artificial Sequence
  Description of Artificial Sequence Synthetic peptide
WMGWVAPYSGNTN

SEQ ID NO: 293 - protein, 15 aa, Artificial Sequence
  Description of Artificial Sequence Synthetic peptide
ARDAGTYSPYGYGMD

SEQ ID NO: 294 - protein, 7 aa, Artificial Sequence
  Description of Artificial Sequence Synthetic peptide
DSWLAWY

SEQ ID NO: 295 - protein, 10 aa, Artificial Sequence
  Description of Artificial Sequence Synthetic peptide
LLIYSASYLE

SEQ ID NO: 296 - protein, 9 aa, Artificial Sequence
  Description of Artificial Sequence Synthetic peptide
QRFQSLPPF

SEQ ID NO: 297 - protein, 8 aa, Artificial Sequence
  Description of Artificial Sequence Synthetic peptide
GYTFRSYG

SEQ ID NO: 298 - protein, 8 aa, Artificial Sequence
  Description of Artificial Sequence Synthetic peptide
VAPYSGNT

SEQ ID NO: 299 - protein, 16 aa, Artificial Sequence
  Description of Artificial Sequence Synthetic peptide
ARDAGTYSPYGYGMDV

SEQ ID NO: 300 - protein, 6 aa, Artificial Sequence
  Description of Artificial Sequence Synthetic peptide
QSIDSW

SEQ ID NO: 301 - protein, 3 aa, Artificial Sequence
  Description of Artificial Sequence Synthetic peptide
SAS

SEQ ID NO: 302 - protein, 10 aa, Artificial Sequence
  Description of Artificial Sequence Synthetic peptide
QRFQSLPPFT

SEQ ID NO: 303 - protein, 123 aa, Artificial Sequence; identical to SEQ ID NO: 265
  Description of Artificial Sequence Synthetic polypeptide

SEQ ID NO: 304 - protein, 108 aa, Artificial Sequence
  Description of Artificial Sequence Synthetic polypeptide
DIQMTQSPSTLSASVGDRVTITCQASQSIDSWLAWYQQKPGKAPKLLIYSASYLESGVPSRFSGSGSGTEFTLTISSLQPDDFATYYCQRFQSLPPFTFGGGTKVEIK

SEQ ID NO: 305 - protein, 10 aa, Artificial Sequence
  Description of Artificial Sequence Synthetic peptide
GFTFHSRGMH

SEQ ID NO: 306 - protein, 17 aa, Artificial Sequence
  Description of Artificial Sequence Synthetic peptide
VITYDGINKYYADSVEG

SEQ ID NO: 307 - protein, 10 aa, Artificial Sequence
  Description of Artificial Sequence Synthetic peptide
DGVYYGVYDY

SEQ ID NO: 308 - protein, 17 aa, Artificial Sequence
  Description of Artificial Sequence Synthetic peptide
KSSQSVLFSSNNKNYLA

SEQ ID NO: 309 - protein, 7 aa, Artificial Sequence
  Description of Artificial Sequence Synthetic peptide
WASTRES

SEQ ID NO: 310 - protein, 9 aa, Artificial Sequence
  Description of Artificial Sequence Synthetic peptide
QQFHSYPLT

SEQ ID NO: 311 - protein, 5 aa, Artificial Sequence
  Description of Artificial Sequence Synthetic peptide
SRGMH

SEQ ID NO: 312 - protein, 17 aa, Artificial Sequence
  Description of Artificial Sequence Synthetic peptide
VITYDGINKYYADSVEG

SEQ ID NO: 313 - protein, 10 aa, Artificial Sequence
  Description of Artificial Sequence Synthetic peptide
DGVYYGVYDY

SEQ ID NO: 314 - protein, 17 aa, Artificial Sequence
  Description of Artificial Sequence Synthetic peptide
KSSQSVLFSSNNKNYLA

SEQ ID NO: 315 - protein, 7 aa, Artificial Sequence
  Description of Artificial Sequence Synthetic peptide
WASTRES

SEQ ID NO: 316 - protein, 9 aa, Artificial Sequence
  Description of Artificial Sequence Synthetic peptide
QQFHSYPLT

SEQ ID NO: 317 - protein, 7 aa, Artificial Sequence
  Description of Artificial Sequence Synthetic peptide
GFTFHSR

SEQ ID NO: 318 - protein, 4 aa, Artificial Sequence
  Description of Artificial Sequence Synthetic peptide
YDGI

SEQ ID NO: 319 - protein, 8 aa, Artificial Sequence
  Description of Artificial Sequence Synthetic peptide
GVYYGVYD

SEQ ID NO: 320 - protein, 13 aa, Artificial Sequence
  Description of Artificial Sequence Synthetic peptide
SQSVLFSSNNKNY

SEQ ID NO: 321 - protein, 3 aa, Artificial Sequence
  Description of Artificial Sequence Synthetic peptide
WAS

SEQ ID NO: 322 - protein, 6 aa, Artificial Sequence
  Description of Artificial Sequence Synthetic peptide
FHSYPL

SEQ ID NO: 323 - protein, 10 aa, Artificial Sequence
  Description of Artificial Sequence Synthetic peptide
GFTFHSRGMH

SEQ ID NO: 324 - protein, 10 aa, Artificial Sequence
  Description of Artificial Sequence Synthetic peptide
VITYDGINKY

SEQ ID NO: 325 - protein, 10 aa, Artificial Sequence
  Description of Artificial Sequence Synthetic peptide
DGVYYGVYDY

SEQ ID NO: 326 - protein, 17 aa, Artificial Sequence
  Description of Artificial Sequence Synthetic peptide
KSSQSVLFSSNNKNYLA

SEQ ID NO: 327 - protein, 7 aa, Artificial Sequence
  Description of Artificial Sequence Synthetic peptide
WASTRES

SEQ ID NO: 328 - protein, 9 aa, Artificial Sequence
  Description of Artificial Sequence Synthetic peptide
QQFHSYPLT

SEQ ID NO: 329 - protein, 6 aa, Artificial Sequence
  Description of Artificial Sequence Synthetic peptide
HSRGMH

SEQ ID NO: 330 - protein, 13 aa, Artificial Sequence
  Description of Artificial Sequence Synthetic peptide
WVAVITYDGINKY

SEQ ID NO: 331 - protein, 11 aa, Artificial Sequence
  Description of Artificial Sequence Synthetic peptide
ARDGVYYGVYD

SEQ ID NO: 332 - protein, 13 aa, Artificial Sequence
  Description of Artificial Sequence Synthetic peptide
LFSSNNKNYLAWY

SEQ ID NO: 333 - protein, 10 aa, Artificial Sequence
  Description of Artificial Sequence Synthetic peptide
LLIYWASTRE

SEQ ID NO: 334 - protein, 8 aa, Artificial Sequence
  Description of Artificial Sequence Synthetic peptide
QQFHSYPL

SEQ ID NO: 335 - protein, 8 aa, Artificial Sequence
  Description of Artificial Sequence Synthetic peptide
GFTFHSRG

SEQ ID NO: 336 - protein, 8 aa, Artificial Sequence
  Description of Artificial Sequence Synthetic peptide
ITYDGINK

SEQ ID NO: 337 - protein, 12 aa, Artificial Sequence
  Description of Artificial Sequence Synthetic peptide
ARDGVYYGVYDY

SEQ ID NO: 338 - protein, 12 aa, Artificial Sequence
  Description of Artificial Sequence Synthetic peptide
QSVLFSSNNKNY

SEQ ID NO: 339 - protein, 3 aa, Artificial Sequence
  Description of Artificial Sequence Synthetic peptide
WAS

SEQ ID NO: 340 - protein, 9 aa, Artificial Sequence
  Description of Artificial Sequence Synthetic peptide
QQFHSYPLT

SEQ ID NO: 341 - protein, 119 aa, Artificial Sequence
  Description of Artificial Sequence Synthetic polypeptide
QVQLVESGGGVVQPGRSLRLSCAASGFTFHSRGMHWVRQAPGKGLEWVAVITYDGINKYYADSVEGRFTISRDNSKNTLYLQMNSLRAEDTAVYYCARDGVYYGVYDYWGQGTLVTVSS

SEQ ID NO: 342 - protein, 113 aa, Artificial Sequence
  Description of Artificial Sequence Synthetic polypeptide
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQFHSYPLTFGGGTKVEIK

SEQ ID NO: 343 - protein, 10 aa, Artificial Sequence
  Description of Artificial Sequence Synthetic peptide
GFTFRSYGMH

SEQ ID NO: 344 - protein, 17 aa, Artificial Sequence
  Description of Artificial Sequence Synthetic peptide
VITYDGINKYYADSVEG

SEQ ID NO: 345 - protein, 10 aa, Artificial Sequence
  Description of Artificial Sequence Synthetic peptide
DGVYYGVYDY

SEQ ID NO: 346 - protein, 17 aa, Artificial Sequence
  Description of Artificial Sequence Synthetic peptide
KSSQSVLFSSNNKNYLA

SEQ ID NO: 347 - protein, 7 aa, Artificial Sequence
  Description of Artificial Sequence Synthetic peptide
WASTRES

SEQ ID NO: 348 - protein, 9 aa, Artificial Sequence
  Description of Artificial Sequence Synthetic peptide
QQFHSYPLT

SEQ ID NO: 349 - protein, 5 aa, Artificial Sequence
  Description of Artificial Sequence Synthetic peptide
SYGMH

SEQ ID NO: 350 - protein, 17 aa, Artificial Sequence
  Description of Artificial Sequence Synthetic peptide
VITYDGINKYYADSVEG

SEQ ID NO: 351 - protein, 10 aa, Artificial Sequence
  Description of Artificial Sequence Synthetic peptide
DGVYYGVYDY

SEQ ID NO: 352 - protein, 17 aa, Artificial Sequence
  Description of Artificial Sequence Synthetic peptide
KSSQSVLFSSNNKNYLA

SEQ ID NO: 353 - protein, 7 aa, Artificial Sequence
  Description of Artificial Sequence Synthetic peptide
WASTRES

SEQ ID NO: 354 - protein, 9 aa, Artificial Sequence
  Description of Artificial Sequence Synthetic peptide
QQFHSYPLT

SEQ ID NO: 355 - protein, 7 aa, Artificial Sequence
  Description of Artificial Sequence Synthetic peptide
GFTFRSY

SEQ ID NO: 356 - protein, 4 aa, Artificial Sequence
  Description of Artificial Sequence Synthetic peptide
YDGI

SEQ ID NO: 357 - protein, 8 aa, Artificial Sequence
  Description of Artificial Sequence Synthetic peptide
GVYYGVYD

SEQ ID NO: 358 - protein, 13 aa, Artificial Sequence
  Description of Artificial Sequence Synthetic peptide
SQSVLFSSNNKNY

SEQ ID NO: 359 - protein, 3 aa, Artificial Sequence
  Description of Artificial Sequence Synthetic peptide
WAS

SEQ ID NO: 360 - protein, 6 aa, Artificial Sequence
  Description of Artificial Sequence Synthetic peptide
FHSYPL

SEQ ID NO: 361 - protein, 10 aa, Artificial Sequence
  Description of Artificial Sequence Synthetic peptide
GFTFRSYGMH

SEQ ID NO: 362 - protein, 10 aa, Artificial Sequence
  Description of Artificial Sequence Synthetic peptide
VITYDGINKY

SEQ ID NO: 363 - protein, 10 aa, Artificial Sequence
  Description of Artificial Sequence Synthetic peptide
DGVYYGVYDY

SEQ ID NO: 364 - protein, 17 aa, Artificial Sequence
  Description of Artificial Sequence Synthetic peptide
KSSQSVLFSSNNKNYLA

SEQ ID NO: 365 - protein, 7 aa, Artificial Sequence
  Description of Artificial Sequence Synthetic peptide
WASTRES

SEQ ID NO: 366 - protein, 9 aa, Artificial Sequence
  Description of Artificial Sequence Synthetic peptide
QQFHSYPLT

SEQ ID NO: 367 - protein, 6 aa, Artificial Sequence
  Description of Artificial Sequence Synthetic peptide
RSYGMH

SEQ ID NO: 368 - protein, 13 aa, Artificial Sequence
  Description of Artificial Sequence Synthetic peptide
WVAVITYDGINKY

SEQ ID NO: 369 - protein, 11 aa, Artificial Sequence
  Description of Artificial Sequence Synthetic peptide
ARDGVYYGVYD

SEQ ID NO: 370 - protein, 13 aa, Artificial Sequence
  Description of Artificial Sequence Synthetic peptide
LFSSNNKNYLAWY

SEQ ID NO: 371 - protein, 10 aa, Artificial Sequence
  Description of Artificial Sequence Synthetic peptide
LLIYWASTRE

SEQ ID NO: 372 - protein, 8 aa, Artificial Sequence
  Description of Artificial Sequence Synthetic peptide
QQFHSYPL

SEQ ID NO: 373 - protein, 8 aa, Artificial Sequence
  Description of Artificial Sequence Synthetic peptide
GFTFRSYG

SEQ ID NO: 374 - protein, 8 aa, Artificial Sequence
  Description of Artificial Sequence Synthetic peptide
ITYDGINK

SEQ ID NO: 375 - protein, 12 aa, Artificial Sequence
  Description of Artificial Sequence Synthetic peptide
ARDGVYYGVYDY

SEQ ID NO: 376 - protein, 12 aa, Artificial Sequence
  Description of Artificial Sequence Synthetic peptide
QSVLFSSNNKNY

SEQ ID NO: 377 - protein, 3 aa, Artificial Sequence
  Description of Artificial Sequence Synthetic peptide
WAS

SEQ ID NO: 378 - protein, 9 aa, Artificial Sequence
  Description of Artificial Sequence Synthetic peptide
QQFHSYPLT

SEQ ID NO: 379 - protein, 119 aa, Artificial Sequence
  Description of Artificial Sequence Synthetic polypeptide
QVQLVESGGGVVQPGRSLRLSCAASGFTFRSYGMHWVRQAPGKGLEWVAVITYDGINKYYADSVEGRFTISRDNSKNTLYLQMNSLRAEDTAVYYCARDGVYYGVYDYWGQGTLVTVSS

SEQ ID NO: 380 - protein, 113 aa, Artificial Sequence; identical to SEQ ID NO: 342
  Description of Artificial Sequence Synthetic polypeptide

SEQ ID NO: 381 - protein, 10 aa, Artificial Sequence
  Description of Artificial Sequence Synthetic peptide
GGTFSSNAIG

SEQ ID NO: 382 - protein, 17 aa, Artificial Sequence
  Description of Artificial Sequence Synthetic peptide
SIIPIIGFANYAQKFQG

SEQ ID NO: 383 - protein, 15 aa, Artificial Sequence
  Description of Artificial Sequence Synthetic peptide
DSGYYYGASSFGMDV

SEQ ID NO: 384 - protein, 11 aa, Artificial Sequence
  Description of Artificial Sequence Synthetic peptide
RASQSVSSNLA

SEQ ID NO: 385 - protein, 7 aa, Artificial Sequence
  Description of Artificial Sequence Synthetic peptide
GASTRAT

SEQ ID NO: 386 - protein, 9 aa, Artificial Sequence
  Description of Artificial Sequence Synthetic peptide
EQYNNLPLT

SEQ ID NO: 387 - protein, 5 aa, Artificial Sequence
  Description of Artificial Sequence Synthetic peptide
SNAIG

SEQ ID NO: 388 - protein, 17 aa, Artificial Sequence
  Description of Artificial Sequence Synthetic peptide
SIIPIIGFANYAQKFQG

SEQ ID NO: 389 - protein, 15 aa, Artificial Sequence
  Description of Artificial Sequence Synthetic peptide
DSGYYYGASSFGMDV

SEQ ID NO: 390 - protein, 11 aa, Artificial Sequence
  Description of Artificial Sequence Synthetic peptide
RASQSVSSNLA

SEQ ID NO: 391 - protein, 7 aa, Artificial Sequence
  Description of Artificial Sequence Synthetic peptide
GASTRAT

SEQ ID NO: 392 - protein, 9 aa, Artificial Sequence
  Description of Artificial Sequence Synthetic peptide
EQYNNLPLT

SEQ ID NO: 393 - protein, 7 aa, Artificial Sequence
  Description of Artificial Sequence Synthetic peptide
GGTFSSN

SEQ ID NO: 394 - protein, 4 aa, Artificial Sequence
  Description of Artificial Sequence Synthetic peptide
PIIG

SEQ ID NO: 395 - protein, 13 aa, Artificial Sequence
  Description of Artificial Sequence Synthetic peptide
SGYYYGASSFGMD

SEQ ID NO: 396 - protein, 7 aa, Artificial Sequence
  Description of Artificial Sequence Synthetic peptide
SQSVSSN

SEQ ID NO: 397 - protein, 3 aa, Artificial Sequence
  Description of Artificial Sequence Synthetic peptide
GAS

SEQ ID NO: 398 - protein, 6 aa, Artificial Sequence
  Description of Artificial Sequence Synthetic peptide
YNNLPL

SEQ ID NO: 399 - protein, 10 aa, Artificial Sequence
  Description of Artificial Sequence Synthetic peptide
GGTFSSNAIG

SEQ ID NO: 400 - protein, 10 aa, Artificial Sequence
  Description of Artificial Sequence Synthetic peptide
SIIPIIGFAN

SEQ ID NO: 401 - protein, 15 aa, Artificial Sequence
  Description of Artificial Sequence Synthetic peptide
DSGYYYGASSFGMDV

SEQ ID NO: 402 - protein, 11 aa, Artificial Sequence
  Description of Artificial Sequence Synthetic peptide
RASQSVSSNLA

SEQ ID NO: 403 - protein, 7 aa, Artificial Sequence
  Description of Artificial Sequence Synthetic peptide
GASTRAT

SEQ ID NO: 404 - protein, 9 aa, Artificial Sequence
  Description of Artificial Sequence Synthetic peptide
EQYNNLPLT

SEQ ID NO: 405 - protein, 6 aa, Artificial Sequence
  Description of Artificial Sequence Synthetic peptide
SSNAIG

SEQ ID NO: 406 - protein, 13 aa, Artificial Sequence
  Description of Artificial Sequence Synthetic peptide
WMGSIIPIIGFAN

SEQ ID NO: 407 - protein, 16 aa, Artificial Sequence
  Description of Artificial Sequence Synthetic peptide
ARDSGYYYGASSFGMD

SEQ ID NO: 408 - protein, 7 aa, Artificial Sequence
  Description of Artificial Sequence Synthetic peptide
SSNLAWY

SEQ ID NO: 409 - protein, 10 aa, Artificial Sequence
  Description of Artificial Sequence Synthetic peptide
LLIYGASTRA

SEQ ID NO: 410 - protein, 8 aa, Artificial Sequence
  Description of Artificial Sequence Synthetic peptide
EQYNNLPL

SEQ ID NO: 411 - protein, 8 aa, Artificial Sequence
  Description of Artificial Sequence Synthetic peptide
GGTFSSNA

SEQ ID NO: 412 - protein, 8 aa, Artificial Sequence
  Description of Artificial Sequence Synthetic peptide
IIPIIGFA

SEQ ID NO: 413 - protein, 17 aa, Artificial Sequence
  Description of Artificial Sequence Synthetic peptide
ARDSGYYYGASSFGMDV

SEQ ID NO: 414 - protein, 6 aa, Artificial Sequence
  Description of Artificial Sequence Synthetic peptide
QSVSSN

SEQ ID NO: 415 - protein, 3 aa, Artificial Sequence
  Description of Artificial Sequence Synthetic peptide
GAS

SEQ ID NO: 416 - protein, 9 aa, Artificial Sequence
  Description of Artificial Sequence Synthetic peptide
EQYNNLPLT

SEQ ID NO: 417 - protein, 124 aa, Artificial Sequence
  Description of Artificial Sequence Synthetic polypeptide
QVQLVQSGAEVKKPGSSVKVSCKASGGTFSSNAIGWVRQAPGQGLEWMGSIIPIIGFANYAQKFQGRVTITADESTSTAYMELSSLRSEDTAVYYCARDSGYYYGASSFGMDVWGQGTTVTVSS

SEQ ID NO: 418 - protein, 107 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCEQYNNLPLTFGGGTKVEIK

SEQ ID NO: 419 - protein, 11 aa, Artificial Sequence
  Description of Artificial Sequence Synthetic peptide
GGSISSGQYWS

SEQ ID NO: 420 - protein, 16 aa, Artificial Sequence
  Description of Artificial Sequence Synthetic peptide
EIYYSGSTRYNPSLKS

SEQ ID NO: 421 - protein, 16 aa, Artificial Sequence
  Description of Artificial Sequence Synthetic peptide
DAPYYYGGGYYYYMDV

SEQ ID NO: 422 - protein, 12 aa, Artificial Sequence
  Description of Artificial Sequence Synthetic peptide
RASQSVSSSYLA

SEQ ID NO: 423 - protein, 7 aa, Artificial Sequence
  Description of Artificial Sequence Synthetic peptide
GASSRAT

SEQ ID NO: 424 - protein, 9 aa, Artificial Sequence
  Description of Artificial Sequence Synthetic peptide
QQVGVVPYT

SEQ ID NO: 425 - protein, 6 aa, Artificial Sequence
  Description of Artificial Sequence Synthetic peptide
SGQYWS

SEQ ID NO: 426 - protein, 16 aa, Artificial Sequence
  Description of Artificial Sequence Synthetic peptide
EIYYSGSTRYNPSLKS

SEQ ID NO: 427 - protein, 16 aa, Artificial Sequence
  Description of Artificial Sequence Synthetic peptide
DAPYYYGGGYYYYMDV

SEQ ID NO: 428 - protein, 12 aa, Artificial Sequence
  Description of Artificial Sequence Synthetic peptide
RASQSVSSSYLA

SEQ ID NO: 429 - protein, 7 aa, Artificial Sequence
  Description of Artificial Sequence Synthetic peptide
GASSRAT

SEQ ID NO: 430 - protein, 9 aa, Artificial Sequence
  Description of Artificial Sequence Synthetic peptide
QQVGVVPYT

SEQ ID NO: 431 - protein, 8 aa, Artificial Sequence
  Description of Artificial Sequence Synthetic peptide
GGSISSGQ

SEQ ID NO: 432 - protein, 3 aa, Artificial Sequence
  Description of Artificial Sequence Synthetic peptide
YSG

SEQ ID NO: 433 - protein, 14 aa, Artificial Sequence
  Description of Artificial Sequence Synthetic peptide
APYYYGGGYYYYMD

SEQ ID NO: 434 - protein, 8 aa, Artificial Sequence
  Description of Artificial Sequence Synthetic peptide
SQSVSSSY

SEQ ID NO: 435 - protein, 3 aa, Artificial Sequence
  Description of Artificial Sequence Synthetic peptide
GAS

SEQ ID NO: 436 - protein, 6 aa, Artificial Sequence
  Description of Artificial Sequence Synthetic peptide
VGVVPY

SEQ ID NO: 437 - protein, 11 aa, Artificial Sequence
  Description of Artificial Sequence Synthetic peptide
GGSISSGQYWS

SEQ ID NO: 438 - protein, 9 aa, Artificial Sequence
  Description of Artificial Sequence Synthetic peptide
EIYYSGSTR

SEQ ID NO: 439 - protein, 16 aa, Artificial Sequence
  Description of Artificial Sequence Synthetic peptide
DAPYYYGGGYYYYMDV

SEQ ID NO: 440 - protein, 12 aa, Artificial Sequence
  Description of Artificial Sequence Synthetic peptide
RASQSVSSSYLA

SEQ ID NO: 441 - protein, 7 aa, Artificial Sequence
  Description of Artificial Sequence Synthetic peptide
GASSRAT

SEQ ID NO: 442 - protein, 9 aa, Artificial Sequence
  Description of Artificial Sequence Synthetic peptide
QQVGVVPYT

SEQ ID NO: 443 - protein, 7 aa, Artificial Sequence
  Description of Artificial Sequence Synthetic peptide
SSGQYWS

SEQ ID NO: 444 - protein, 12 aa, Artificial Sequence
  Description of Artificial Sequence Synthetic peptide
WIGEIYYSGSTR

SEQ ID NO: 445 - protein, 17 aa, Artificial Sequence
  Description of Artificial Sequence Synthetic peptide
ARDAPYYYGGGYYYYMD

SEQ ID NO: 446 - protein, 8 aa, Artificial Sequence
  Description of Artificial Sequence Synthetic peptide
SSSYLAWY

SEQ ID NO: 447 - protein, 10 aa, Artificial Sequence
  Description of Artificial Sequence Synthetic peptide
LLIYGASSRA

SEQ ID NO: 448 - protein, 8 aa, Artificial Sequence
  Description of Artificial Sequence Synthetic peptide
QQVGVVPY

SEQ ID NO: 449 - protein, 9 aa, Artificial Sequence
  Description of Artificial Sequence Synthetic peptide
GGSISSGQY

SEQ ID NO: 450 - protein, 7 aa, Artificial Sequence
  Description of Artificial Sequence Synthetic peptide
IYYSGST

SEQ ID NO: 451 - protein, 18 aa, Artificial Sequence
  Description of Artificial Sequence Synthetic peptide
ARDAPYYYGGGYYYYMDV

SEQ ID NO: 452 - protein, 7 aa, Artificial Sequence
  Description of Artificial Sequence Synthetic peptide
QSVSSSY

SEQ ID NO: 453 - protein, 3 aa, Artificial Sequence
  Description of Artificial Sequence Synthetic peptide
GAS

SEQ ID NO: 454 - protein, 9 aa, Artificial Sequence
  Description of Artificial Sequence Synthetic peptide
QQVGVVPYT

SEQ ID NO: 455 - protein, 125 aa, Artificial Sequence
  Description of Artificial Sequence Synthetic polypeptide
QVQLQESGPGLVKPSQTLSLTCTVSGGSISSGQYWSWIRQHPGKGLEWIGEIYYSGSTRYNPSLKSRVTISVDTSKNQFSLKLSSVTAADTAVYYCARDAPYYYGGGYYYYMDVWGKGTTVTVSS

SEQ ID NO: 456 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQVGVVPYTFGGGTKVEIK

SEQ ID NO: 457 - protein, 11 aa, Artificial Sequence
  Description of Artificial Sequence Synthetic peptide
GGSISSGQYWS

SEQ ID NO: 458 - protein, 16 aa, Artificial Sequence
  Description of Artificial Sequence Synthetic peptide
EIYYSGSTRYNPSLKS

SEQ ID NO: 459 - protein, 16 aa, Artificial Sequence
  Description of Artificial Sequence Synthetic peptide
DAPYYYGGGYYYYMDV

SEQ ID NO: 460 - protein, 12 aa, Artificial Sequence
  Description of Artificial Sequence Synthetic peptide
RASESVDSSYLA

SEQ ID NO: 461 - protein, 7 aa, Artificial Sequence
  Description of Artificial Sequence Synthetic peptide
GASTRQT

SEQ ID NO: 462 - protein, 9 aa, Artificial Sequence
  Description of Artificial Sequence Synthetic peptide
QQAGVVPYT

SEQ ID NO: 463 - protein, 6 aa, Artificial Sequence
  Description of Artificial Sequence Synthetic peptide
SGQYWS

SEQ ID NO: 464 - protein, 16 aa, Artificial Sequence
  Description of Artificial Sequence Synthetic peptide
EIYYSGSTRYNPSLKS

SEQ ID NO: 465 - protein, 16 aa, Artificial Sequence
  Description of Artificial Sequence Synthetic peptide
DAPYYYGGGYYYYMDV

SEQ ID NO: 466 - protein, 12 aa, Artificial Sequence
  Description of Artificial Sequence Synthetic peptide
RASESVDSSYLA

SEQ ID NO: 467 - protein, 7 aa, Artificial Sequence
  Description of Artificial Sequence Synthetic peptide
GASTRQT

SEQ ID NO: 468 - protein, 9 aa, Artificial Sequence
  Description of Artificial Sequence Synthetic peptide
QQAGVVPYT

SEQ ID NO: 469 - protein, 8 aa, Artificial Sequence
  Description of Artificial Sequence Synthetic peptide
GGSISSGQ

SEQ ID NO: 470 - protein, 3 aa, Artificial Sequence
  Description of Artificial Sequence Synthetic peptide
YSG

SEQ ID NO: 471 - protein, 14 aa, Artificial Sequence
  Description of Artificial Sequence Synthetic peptide
APYYYGGGYYYYMD

SEQ ID NO: 472 - protein, 8 aa, Artificial Sequence
  Description of Artificial Sequence Synthetic peptide
SESVDSSY

SEQ ID NO: 473 - protein, 3 aa, Artificial Sequence
  Description of Artificial Sequence Synthetic peptide
GAS

SEQ ID NO: 474 - protein, 6 aa, Artificial Sequence
  Description of Artificial Sequence Synthetic peptide
AGVVPY

SEQ ID NO: 475 - protein, 11 aa, Artificial Sequence
  Description of Artificial Sequence Synthetic peptide
GGSISSGQYWS

SEQ ID NO: 476 - protein, 9 aa, Artificial Sequence
  Description of Artificial Sequence Synthetic peptide
EIYYSGSTR

SEQ ID NO: 477 - protein, 16 aa, Artificial Sequence
  Description of Artificial Sequence Synthetic peptide
DAPYYYGGGYYYYMDV

SEQ ID NO: 478 - protein, 12 aa, Artificial Sequence
  Description of Artificial Sequence Synthetic peptide
RASESVDSSYLA

SEQ ID NO: 479 - protein, 7 aa, Artificial Sequence
  Description of Artificial Sequence Synthetic peptide
GASTRQT

SEQ ID NO: 480 - protein, 9 aa, Artificial Sequence
  Description of Artificial Sequence Synthetic peptide
QQAGVVPYT

SEQ ID NO: 481 - protein, 7 aa, Artificial Sequence
  Description of Artificial Sequence Synthetic peptide
SSGQYWS

SEQ ID NO: 482 - protein, 12 aa, Artificial Sequence
  Description of Artificial Sequence Synthetic peptide
WIGEIYYSGSTR

SEQ ID NO: 483 - protein, 17 aa, Artificial Sequence
  Description of Artificial Sequence Synthetic peptide
ARDAPYYYGGGYYYYMD

SEQ ID NO: 484 - protein, 8 aa, Artificial Sequence
  Description of Artificial Sequence Synthetic peptide
DSSYLAWY

SEQ ID NO: 485 - protein, 10 aa, Artificial Sequence
  Description of Artificial Sequence Synthetic peptide
LLIYGASTRQ

SEQ ID NO: 486 - protein, 8 aa, Artificial Sequence
  Description of Artificial Sequence Synthetic peptide
QQAGVVPY

SEQ ID NO: 487 - protein, 9 aa, Artificial Sequence
  Description of Artificial Sequence Synthetic peptide
GGSISSGQY

SEQ ID NO: 488 - protein, 7 aa, Artificial Sequence
  Description of Artificial Sequence Synthetic peptide
IYYSGST

SEQ ID NO: 489 - protein, 18 aa, Artificial Sequence
  Description of Artificial Sequence Synthetic peptide
ARDAPYYYGGGYYYYMDV

SEQ ID NO: 490 - protein, 7 aa, Artificial Sequence
  Description of Artificial Sequence Synthetic peptide
ESVDSSY

SEQ ID NO: 491 - protein, 3 aa, Artificial Sequence
  Description of Artificial Sequence Synthetic peptide
GAS

SEQ ID NO: 492 - protein, 9 aa, Artificial Sequence
  Description of Artificial Sequence Synthetic peptide
QQAGVVPYT

SEQ ID NO: 493 - protein, 125 aa, Artificial Sequence; identical to SEQ ID NO: 455
  Description of Artificial Sequence Synthetic polypeptide

SEQ ID NO: 494 - protein, 108 aa, Artificial Sequence
  Description of Artificial Sequence Synthetic polypeptide
EIVLTQSPGTLSLSPGERATLSCRASESVDSSYLAWYQQKPGQAPRLLIYGASTRQTGIPDRFSGSGSGTDFTLTISRLEPEDFAVYYCQQAGVVPYTFGGGTKVEIK

SEQ ID NO: 495 - protein, 11 aa, Artificial Sequence
  Description of Artificial Sequence Synthetic peptide
GGSISSGQYWS

SEQ ID NO: 496 - protein, 16 aa, Artificial Sequence
  Description of Artificial Sequence Synthetic peptide
EIYYSGSTRYNPSLKS

SEQ ID NO: 497 - protein, 16 aa, Artificial Sequence
  Description of Artificial Sequence Synthetic peptide
DAPYYYGGGYYYYMDV

SEQ ID NO: 498 - protein, 12 aa, Artificial Sequence
  Description of Artificial Sequence Synthetic peptide
RASESVDSSYLA

SEQ ID NO: 499 - protein, 7 aa, Artificial Sequence
  Description of Artificial Sequence Synthetic peptide
GADSRAT

SEQ ID NO: 500 - protein, 9 aa, Artificial Sequence
  Description of Artificial Sequence Synthetic peptide
QQDGVVPYT

SEQ ID NO: 501 - protein, 6 aa, Artificial Sequence
  Description of Artificial Sequence Synthetic peptide
SGQYWS

SEQ ID NO: 502 - protein, 16 aa, Artificial Sequence
  Description of Artificial Sequence Synthetic peptide
EIYYSGSTRYNPSLKS

SEQ ID NO: 503 - protein, 16 aa, Artificial Sequence
  Description of Artificial Sequence Synthetic peptide
DAPYYYGGGYYYYMDV

SEQ ID NO: 504 - protein, 12 aa, Artificial Sequence
  Description of Artificial Sequence Synthetic peptide
RASESVDSSYLA

SEQ ID NO: 505 - protein, 7 aa, Artificial Sequence
  Description of Artificial Sequence Synthetic peptide
GADSRAT

SEQ ID NO: 506 - protein, 9 aa, Artificial Sequence
  Description of Artificial Sequence Synthetic peptide
QQDGVVPYT

SEQ ID NO: 507 - protein, 8 aa, Artificial Sequence
  Description of Artificial Sequence Synthetic peptide
GGSISSGQ

SEQ ID NO: 508 - protein, 3 aa, Artificial Sequence
  Description of Artificial Sequence Synthetic peptide
YSG

SEQ ID NO: 509 - protein, 14 aa, Artificial Sequence
  Description of Artificial Sequence Synthetic peptide
APYYYGGGYYYYMD

SEQ ID NO: 510 - protein, 8 aa, Artificial Sequence
  Description of Artificial Sequence Synthetic peptide
SESVDSSY

SEQ ID NO: 511 - protein, 3 aa, Artificial Sequence
  Description of Artificial Sequence Synthetic peptide
GAD

SEQ ID NO: 512 - protein, 6 aa, Artificial Sequence
  Description of Artificial Sequence Synthetic peptide
DGVVPY

SEQ ID NO: 513 - protein, 11 aa, Artificial Sequence
  Description of Artificial Sequence Synthetic peptide
GGSISSGQYWS

SEQ ID NO: 514 - protein, 9 aa, Artificial Sequence
  Description of Artificial Sequence Synthetic peptide
EIYYSGSTR

SEQ ID NO: 515 - protein, 16 aa, Artificial Sequence
  Description of Artificial Sequence Synthetic peptide
DAPYYYGGGYYYYMDV

SEQ ID NO: 516 - protein, 12 aa, Artificial Sequence
  Description of Artificial Sequence Synthetic peptide
RASESVDSSYLA

SEQ ID NO: 517 - protein, 7 aa, Artificial Sequence
  Description of Artificial Sequence Synthetic peptide
GADSRAT

SEQ ID NO: 518 - protein, 9 aa, Artificial Sequence
  Description of Artificial Sequence Synthetic peptide
QQDGVVPYT

SEQ ID NO: 519 - protein, 7 aa, Artificial Sequence
  Description of Artificial Sequence Synthetic peptide
SSGQYWS

SEQ ID NO: 520 - protein, 12 aa, Artificial Sequence
  Description of Artificial Sequence Synthetic peptide
WIGEIYYSGSTR

SEQ ID NO: 521 - protein, 17 aa, Artificial Sequence
  Description of Artificial Sequence Synthetic peptide
ARDAPYYYGGGYYYYMD

SEQ ID NO: 522 - protein, 8 aa, Artificial Sequence
  Description of Artificial Sequence Synthetic peptide
DSSYLAWY

SEQ ID NO: 523 - protein, 10 aa, Artificial Sequence
  Description of Artificial Sequence Synthetic peptide
LLIYGADSRA

SEQ ID NO: 524 - protein, 8 aa, Artificial Sequence
  Description of Artificial Sequence Synthetic peptide
QQDGVVPY

SEQ ID NO: 525 - protein, 9 aa, Artificial Sequence
  Description of Artificial Sequence Synthetic peptide
GGSISSGQY

SEQ ID NO: 526 - protein, 7 aa, Artificial Sequence
  Description of Artificial Sequence Synthetic peptide
IYYSGST

SEQ ID NO: 527 - protein, 18 aa, Artificial Sequence
  Description of Artificial Sequence Synthetic peptide
ARDAPYYYGGGYYYYMDV

SEQ ID NO: 528 - protein, 7 aa, Artificial Sequence
  Description of Artificial Sequence Synthetic peptide
ESVDSSY

SEQ ID NO: 529 - protein, 3 aa, Artificial Sequence
  Description of Artificial Sequence Synthetic peptide
GAD

SEQ ID NO: 530 - protein, 9 aa, Artificial Sequence
  Description of Artificial Sequence Synthetic peptide
QQDGVVPYT

SEQ ID NO: 531 - protein, 125 aa, Artificial Sequence; identical to SEQ ID NO: 455
  Description of Artificial Sequence Synthetic polypeptide

SEQ ID NO: 532 - protein, 108 aa, Artificial Sequence
  Description of Artificial Sequence Synthetic polypeptide
EIVLTQSPGTLSLSPGERATLSCRASESVDSSYLAWYQQKPGQAPRLLIYGADSRATGIPDRFSGSGSGTDFTLTISRLEPEDFAVYYCQQDGVVPYTFGGGTKVEIK

SEQ ID NO: 533 - protein, 10 aa, Artificial Sequence
  Description of Artificial Sequence Synthetic peptide
GGSLSGYYWS

SEQ ID NO: 534 - protein, 16 aa, Artificial Sequence
  Description of Artificial Sequence Synthetic peptide
EIGASGSTRYNPSLKS

SEQ ID NO: 535 - protein, 16 aa, Artificial Sequence
  Description of Artificial Sequence Synthetic peptide
DTPYYYEGGYYYYMDV

SEQ ID NO: 536 - protein, 12 aa, Artificial Sequence
  Description of Artificial Sequence Synthetic peptide
RASQSVSSSYLA

SEQ ID NO: 537 - protein, 7 aa, Artificial Sequence
  Description of Artificial Sequence Synthetic peptide
GASSRAT

SEQ ID NO: 538 - protein, 9 aa, Artificial Sequence
  Description of Artificial Sequence Synthetic peptide
QQVGVVPYT

SEQ ID NO: 539 - protein, 5 aa, Artificial Sequence
  Description of Artificial Sequence Synthetic peptide
GYYWS

SEQ ID NO: 540 - protein, 16 aa, Artificial Sequence
  Description of Artificial Sequence Synthetic peptide
EIGASGSTRYNPSLKS

SEQ ID NO: 541 - protein, 16 aa, Artificial Sequence
  Description of Artificial Sequence Synthetic peptide
DTPYYYEGGYYYYMDV

SEQ ID NO: 542 - protein, 12 aa, Artificial Sequence
  Description of Artificial Sequence Synthetic peptide
RASQSVSSSYLA

SEQ ID NO: 543 - protein, 7 aa, Artificial Sequence
  Description of Artificial Sequence Synthetic peptide
GASSRAT

SEQ ID NO: 544 - protein, 9 aa, Artificial Sequence
  Description of Artificial Sequence Synthetic peptide
QQVGVVPYT

SEQ ID NO: 545 - protein, 7 aa, Artificial Sequence
  Description of Artificial Sequence Synthetic peptide
GGSLSGY

SEQ ID NO: 546 - protein, 3 aa, Artificial Sequence
  Description of Artificial Sequence Synthetic peptide
ASG

SEQ ID NO: 547 - protein, 14 aa, Artificial Sequence
  Description of Artificial Sequence Synthetic peptide
TPYYYEGGYYYYMD

SEQ ID NO: 548 - protein, 8 aa, Artificial Sequence
  Description of Artificial Sequence Synthetic peptide
SQSVSSSY

SEQ ID NO: 549 - protein, 3 aa, Artificial Sequence
  Description of Artificial Sequence Synthetic peptide
GAS

SEQ ID NO: 550 - protein, 6 aa, Artificial Sequence
  Description of Artificial Sequence Synthetic peptide
VGVVPY

SEQ ID NO: 551 - protein, 10 aa, Artificial Sequence
  Description of Artificial Sequence Synthetic peptide
GGSLSGYYWS

SEQ ID NO: 552 - protein, 9 aa, Artificial Sequence
  Description of Artificial Sequence Synthetic peptide
EIGASGSTR

SEQ ID NO: 553 - protein, 16 aa, Artificial Sequence
  Description of Artificial Sequence Synthetic peptide
DTPYYYEGGYYYYMDV

SEQ ID NO: 554 - protein, 12 aa, Artificial Sequence
  Description of Artificial Sequence Synthetic peptide
RASQSVSSSYLA

SEQ ID NO: 555 - protein, 7 aa, Artificial Sequence
  Description of Artificial Sequence Synthetic peptide
GASSRAT

SEQ ID NO: 556 - protein, 9 aa, Artificial Sequence
  Description of Artificial Sequence Synthetic peptide
QQVGVVPYT

SEQ ID NO: 557 - protein, 6 aa, Artificial Sequence
  Description of Artificial Sequence Synthetic peptide
SGYYWS

SEQ ID NO: 558 - protein, 12 aa, Artificial Sequence
  Description of Artificial Sequence Synthetic peptide
WIGEIGASGSTR

SEQ ID NO: 559 - protein, 17 aa, Artificial Sequence
  Description of Artificial Sequence Synthetic peptide
ARDTPYYYEGGYYYYMD

SEQ ID NO: 560 - protein, 8 aa, Artificial Sequence
  Description of Artificial Sequence Synthetic peptide
SSSYLAWY

SEQ ID NO: 561 - protein, 10 aa, Artificial Sequence
  Description of Artificial Sequence Synthetic peptide
LLIYGASSRA

SEQ ID NO: 562 - protein, 8 aa, Artificial Sequence
  Description of Artificial Sequence Synthetic peptide
QQVGVVPY

SEQ ID NO: 563 - protein, 8 aa, Artificial Sequence
  Description of Artificial Sequence Synthetic peptide
GGSLSGYY

SEQ ID NO: 564 - protein, 7 aa, Artificial Sequence
  Description of Artificial Sequence Synthetic peptide
IGASGST

SEQ ID NO: 565 - protein, 18 aa, Artificial Sequence
  Description of Artificial Sequence Synthetic peptide
ARDTPYYYEGGYYYYMDV

SEQ ID NO: 566 - protein, 7 aa, Artificial Sequence
  Description of Artificial Sequence Synthetic peptide
QSVSSSY

SEQ ID NO: 567 - protein, 3 aa, Artificial Sequence
  Description of Artificial Sequence Synthetic peptide
GAS

SEQ ID NO: 568 - protein, 9 aa, Artificial Sequence
  Description of Artificial Sequence Synthetic peptide
QQVGVVPYT

SEQ ID NO: 569 - protein, 124 aa, Artificial Sequence
  Description of Artificial Sequence Synthetic polypeptide
QVQLQQWGAGLLKPSETLSLTCAVYGGSLSGYYWSWIRQPPGKGLEWIGEIGASGSTRYNPSLKSRVTISVDTSKNQFSLKLSSVTAADTAVYYCARDTPYYYEGGYYYYMDVWGKGTTVTVSS

SEQ ID NO: 570 - protein, 108 aa, Artificial Sequence; identical to SEQ ID NO: 456
  Description of Artificial Sequence Synthetic polypeptide

SEQ ID NO: 571 - protein, 10 aa, Artificial Sequence
  Description of Artificial Sequence Synthetic peptide
GGSLSGYYWS

SEQ ID NO: 572 - protein, 16 aa, Artificial Sequence
  Description of Artificial Sequence Synthetic peptide
EIGASGSTRYNPSLKS

SEQ ID NO: 573 - protein, 16 aa, Artificial Sequence
  Description of Artificial Sequence Synthetic peptide
DTPYYYEGGYYYYMDV

SEQ ID NO: 574 - protein, 12 aa, Artificial Sequence
  Description of Artificial Sequence Synthetic peptide
RASDSVDSSYLA

SEQ ID NO: 575 - protein, 7 aa, Artificial Sequence
  Description of Artificial Sequence Synthetic peptide
GAFSRAN

SEQ ID NO: 576 - protein, 9 aa, Artificial Sequence
  Description of Artificial Sequence Synthetic peptide
QQAGVVPYT

SEQ ID NO: 577 - protein, 5 aa, Artificial Sequence
  Description of Artificial Sequence Synthetic peptide
GYYWS

SEQ ID NO: 578 - protein, 16 aa, Artificial Sequence
  Description of Artificial Sequence Synthetic peptide
EIGASGSTRYNPSLKS

SEQ ID NO: 579 - protein, 16 aa, Artificial Sequence
  Description of Artificial Sequence Synthetic peptide
DTPYYYEGGYYYYMDV

SEQ ID NO: 580 - protein, 12 aa, Artificial Sequence
  Description of Artificial Sequence Synthetic peptide
RASDSVDSSYLA

SEQ ID NO: 581 - protein, 7 aa, Artificial Sequence
  Description of Artificial Sequence Synthetic peptide
GAFSRAN

SEQ ID NO: 582 - protein, 9 aa, Artificial Sequence
  Description of Artificial Sequence Synthetic peptide
QQAGVVPYT

SEQ ID NO: 583 - protein, 7 aa, Artificial Sequence
  Description of Artificial Sequence Synthetic peptide
GGSLSGY

SEQ ID NO: 584 - protein, 3 aa, Artificial Sequence
  Description of Artificial Sequence Synthetic peptide
ASG

SEQ ID NO: 585 - protein, 14 aa, Artificial Sequence
  Description of Artificial Sequence Synthetic peptide
TPYYYEGGYYYYMD

SEQ ID NO: 586 - protein, 8 aa, Artificial Sequence
  Description of Artificial Sequence Synthetic peptide
SDSVDSSY

SEQ ID NO: 587 - protein, 3 aa, Artificial Sequence
  Description of Artificial Sequence Synthetic peptide
GAF

SEQ ID NO: 588 - protein, 6 aa, Artificial Sequence
  Description of Artificial Sequence Synthetic peptide
AGVVPY

SEQ ID NO: 589 - protein, 10 aa, Artificial Sequence
  Description of Artificial Sequence Synthetic peptide
GGSLSGYYWS

SEQ ID NO: 590 - protein, 9 aa, Artificial Sequence
  Description of Artificial Sequence Synthetic peptide
EIGASGSTR

SEQ ID NO: 591 - protein, 16 aa, Artificial Sequence
  Description of Artificial Sequence Synthetic peptide
DTPYYYEGGYYYYMDV

SEQ ID NO: 592 - protein, 12 aa, Artificial Sequence
  Description of Artificial Sequence Synthetic peptide
RASDSVDSSYLA

SEQ ID NO: 593 - protein, 7 aa, Artificial Sequence
  Description of Artificial Sequence Synthetic peptide
GAFSRAN

SEQ ID NO: 594 - protein, 9 aa, Artificial Sequence
  Description of Artificial Sequence Synthetic peptide
QQAGVVPYT

SEQ ID NO: 595 - protein, 6 aa, Artificial Sequence
  Description of Artificial Sequence Synthetic peptide
SGYYWS

SEQ ID NO: 596 - protein, 12 aa, Artificial Sequence
  Description of Artificial Sequence Synthetic peptide
WIGEIGASGSTR

SEQ ID NO: 597 - protein, 17 aa, Artificial Sequence
  Description of Artificial Sequence Synthetic peptide
ARDTPYYYEGGYYYYMD

SEQ ID NO: 598 - protein, 8 aa, Artificial Sequence
  Description of Artificial Sequence Synthetic peptide
DSSYLAWY

SEQ ID NO: 599 - protein, 10 aa, Artificial Sequence
  Description of Artificial Sequence Synthetic peptide
LLIYGAFSRA

SEQ ID NO: 600 - protein, 8 aa, Artificial Sequence
  Description of Artificial Sequence Synthetic peptide
QQAGVVPY

SEQ ID NO: 601 - protein, 8 aa, Artificial Sequence
  Description of Artificial Sequence Synthetic peptide
GGSLSGYY

SEQ ID NO: 602 - protein, 7 aa, Artificial Sequence
  Description of Artificial Sequence Synthetic peptide
IGASGST

SEQ ID NO: 603 - protein, 18 aa, Artificial Sequence
  Description of Artificial Sequence Synthetic peptide
ARDTPYYYEGGYYYYMDV

SEQ ID NO: 604 - protein, 7 aa, Artificial Sequence
  Description of Artificial Sequence Synthetic peptide
DSVDSSY

SEQ ID NO: 605 - protein, 3 aa, Artificial Sequence
  Description of Artificial Sequence Synthetic peptide
GAF

SEQ ID NO: 606 - protein, 9 aa, Artificial Sequence
  Description of Artificial Sequence Synthetic peptide
QQAGVVPYT

SEQ ID NO: 607 - protein, 124 aa, Artificial Sequence; identical to SEQ ID NO: 569
  Description of Artificial Sequence Synthetic polypeptide

SEQ ID NO: 608 - protein, 108 aa, Artificial Sequence
  Description of Artificial Sequence Synthetic polypeptide
EIVLTQSPGTLSLSPGERATLSCRASDSVDSSYLAWYQQKPGQAPRLLIYGAFSRANGIPDRFSGSGSGTDFTLTISRLEPEDFAVYYCQQAGVVPYTFGGGTKVEIK

SEQ ID NO: 609 - protein, 10 aa, Artificial Sequence
  Description of Artificial Sequence Synthetic peptide
GGSLSGYYWS

SEQ ID NO: 610 - protein, 16 aa, Artificial Sequence
  Description of Artificial Sequence Synthetic peptide
EIGASGSTRYNPSLKS

SEQ ID NO: 611 - protein, 16 aa, Artificial Sequence
  Description of Artificial Sequence Synthetic peptide
DTPYYYEGGYYYYMDV

SEQ ID NO: 612 - protein, 12 aa, Artificial Sequence
  Description of Artificial Sequence Synthetic peptide
RASQSVSSSFLA

SEQ ID NO: 613 - protein, 7 aa, Artificial Sequence
  Description of Artificial Sequence Synthetic peptide
GAYSRAT

SEQ ID NO: 614 - protein, 9 aa, Artificial Sequence
  Description of Artificial Sequence Synthetic peptide
QQAGVVPYT

SEQ ID NO: 615 - protein, 5 aa, Artificial Sequence
  Description of Artificial Sequence Synthetic peptide
GYYWS

SEQ ID NO: 616 - protein, 16 aa, Artificial Sequence
  Description of Artificial Sequence Synthetic peptide
EIGASGSTRYNPSLKS

SEQ ID NO: 617 - protein, 16 aa, Artificial Sequence
  Description of Artificial Sequence Synthetic peptide
DTPYYYEGGYYYYMDV

SEQ ID NO: 618 - protein, 12 aa, Artificial Sequence
  Description of Artificial Sequence Synthetic peptide
RASQSVSSSFLA

SEQ ID NO: 619 - protein, 7 aa, Artificial Sequence
  Description of Artificial Sequence Synthetic peptide
GAYSRAT

SEQ ID NO: 620 - protein, 9 aa, Artificial Sequence
  Description of Artificial Sequence Synthetic peptide
QQAGVVPYT

SEQ ID NO: 621 - protein, 7 aa, Artificial Sequence
  Description of Artificial Sequence Synthetic peptide
GGSLSGY

SEQ ID NO: 622 - protein, 3 aa, Artificial Sequence
  Description of Artificial Sequence Synthetic peptide
ASG

SEQ ID NO: 623 - protein, 14 aa, Artificial Sequence
  Description of Artificial Sequence Synthetic peptide
TPYYYEGGYYYYMD

SEQ ID NO: 624 - protein, 8 aa, Artificial Sequence
  Description of Artificial Sequence Synthetic peptide
SQSVSSSF

SEQ ID NO: 625 - protein, 3 aa, Artificial Sequence
  Description of Artificial Sequence Synthetic peptide
GAY

SEQ ID NO: 626 - protein, 6 aa, Artificial Sequence
  Description of Artificial Sequence Synthetic peptide
AGVVPY

SEQ ID NO: 627 - protein, 10 aa, Artificial Sequence
  Description of Artificial Sequence Synthetic peptide
GGSLSGYYWS

SEQ ID NO: 628 - protein, 9 aa, Artificial Sequence
  Description of Artificial Sequence Synthetic peptide
EIGASGSTR

SEQ ID NO: 629 - protein, 16 aa, Artificial Sequence
  Description of Artificial Sequence Synthetic peptide
DTPYYYEGGYYYYMDV

SEQ ID NO: 630 - protein, 12 aa, Artificial Sequence
  Description of Artificial Sequence Synthetic peptide
RASQSVSSSFLA

SEQ ID NO: 631 - protein, 7 aa, Artificial Sequence
  Description of Artificial Sequence Synthetic peptide
GAYSRAT

SEQ ID NO: 632 - protein, 9 aa, Artificial Sequence
  Description of Artificial Sequence Synthetic peptide
QQAGVVPYT

SEQ ID NO: 633 - protein, 6 aa, Artificial Sequence
  Description of Artificial Sequence Synthetic peptide
SGYYWS

SEQ ID NO: 634 - protein, 12 aa, Artificial Sequence
  Description of Artificial Sequence Synthetic peptide
WIGEIGASGSTR

SEQ ID NO: 635 - protein, 17 aa, Artificial Sequence
  Description of Artificial Sequence Synthetic peptide
ARDTPYYYEGGYYYYMD

SEQ ID NO: 636 - protein, 8 aa, Artificial Sequence
  Description of Artificial Sequence Synthetic peptide
SSSFLAWY

SEQ ID NO: 637 - protein, 10 aa, Artificial Sequence
  Description of Artificial Sequence Synthetic peptide
LLIYGAYSRA

SEQ ID NO: 638 - protein, 8 aa, Artificial Sequence
  Description of Artificial Sequence Synthetic peptide
QQAGVVPY

SEQ ID NO: 639 - protein, 8 aa, Artificial Sequence
  Description of Artificial Sequence Synthetic peptide
GGSLSGYY

SEQ ID NO: 640 - protein, 7 aa, Artificial Sequence
  Description of Artificial Sequence Synthetic peptide
IGASGST

SEQ ID NO: 641 - protein, 18 aa, Artificial Sequence
  Description of Artificial Sequence Synthetic peptide
ARDTPYYYEGGYYYYMDV

SEQ ID NO: 642 - protein, 7 aa, Artificial Sequence
  Description of Artificial Sequence Synthetic peptide
QSVSSSF

SEQ ID NO: 643 - protein, 3 aa, Artificial Sequence
  Description of Artificial Sequence Synthetic peptide
GAY

SEQ ID NO: 644 - protein, 9 aa, Artificial Sequence
  Description of Artificial Sequence Synthetic peptide
QQAGVVPYT

SEQ ID NO: 645 - protein, 124 aa, Artificial Sequence; identical to SEQ ID NO: 569
  Description of Artificial Sequence Synthetic polypeptide

SEQ ID NO: 646 - protein, 108 aa, Artificial Sequence
  Description of Artificial Sequence Synthetic polypeptide
EIVLTQSPGTLSLSPGERATLSCRASQSVSSSFLAWYQQKPGQAPRLLIYGAYSRATGIPDRFSGSGSGTDFTLTISRLEPEDFAVYYCQQAGVVPYTFGGGTKVEIK

SEQ ID NO: 647 - protein, 11 aa, Artificial Sequence
  Description of Artificial Sequence Synthetic peptide
GGSISSGQYWS

SEQ ID NO: 648 - protein, 16 aa, Artificial Sequence
  Description of Artificial Sequence Synthetic peptide
EIYYSGSTRYNPSLKS

SEQ ID NO: 649 - protein, 16 aa, Artificial Sequence
  Description of Artificial Sequence Synthetic peptide
DTPYYYDGGYYYYMDV

SEQ ID NO: 650 - protein, 12 aa, Artificial Sequence
  Description of Artificial Sequence Synthetic peptide
RASQSVSSSYLA

SEQ ID NO: 651 - protein, 7 aa, Artificial Sequence
  Description of Artificial Sequence Synthetic peptide
GASSRAT

SEQ ID NO: 652 - protein, 9 aa, Artificial Sequence
  Description of Artificial Sequence Synthetic peptide
QQVGVVPYT

SEQ ID NO: 653 - protein, 6 aa, Artificial Sequence
  Description of Artificial Sequence Synthetic peptide
SGQYWS

SEQ ID NO: 654 - protein, 16 aa, Artificial Sequence
  Description of Artificial Sequence Synthetic peptide
EIYYSGSTRYNPSLKS

SEQ ID NO: 655 - protein, 16 aa, Artificial Sequence
  Description of Artificial Sequence Synthetic peptide
DTPYYYDGGYYYYMDV

SEQ ID NO: 656 - protein, 12 aa, Artificial Sequence
  Description of Artificial Sequence Synthetic peptide
RASQSVSSSYLA

SEQ ID NO: 657 - protein, 7 aa, Artificial Sequence
  Description of Artificial Sequence Synthetic peptide
GASSRAT

SEQ ID NO: 658 - protein, 9 aa, Artificial Sequence
  Description of Artificial Sequence Synthetic peptide
QQVGVVPYT

SEQ ID NO: 659 - protein, 8 aa, Artificial Sequence
  Description of Artificial Sequence Synthetic peptide
GGSISSGQ

SEQ ID NO: 660 - protein, 3 aa, Artificial Sequence
  Description of Artificial Sequence Synthetic peptide
YSG

SEQ ID NO: 661 - protein, 14 aa, Artificial Sequence
  Description of Artificial Sequence Synthetic peptide
TPYYYDGGYYYYMD

SEQ ID NO: 662 - protein, 8 aa, Artificial Sequence
  Description of Artificial Sequence Synthetic peptide
SQSVSSSY

SEQ ID NO: 663 - protein, 3 aa, Artificial Sequence
  Description of Artificial Sequence Synthetic peptide
GAS

SEQ ID NO: 664 - protein, 6 aa, Artificial Sequence
  Description of Artificial Sequence Synthetic peptide
VGVVPY

SEQ ID NO: 665 - protein, 11 aa, Artificial Sequence
  Description of Artificial Sequence Synthetic peptide
GGSISSGQYWS

SEQ ID NO: 666 - protein, 9 aa, Artificial Sequence
  Description of Artificial Sequence Synthetic peptide
EIYYSGSTR

SEQ ID NO: 667 - protein, 16 aa, Artificial Sequence
  Description of Artificial Sequence Synthetic peptide
DTPYYYDGGYYYYMDV

SEQ ID NO: 668 - protein, 12 aa, Artificial Sequence
  Description of Artificial Sequence Synthetic peptide
RASQSVSSSYLA

SEQ ID NO: 669 - protein, 7 aa, Artificial Sequence
  Description of Artificial Sequence Synthetic peptide
GASSRAT

SEQ ID NO: 670 - protein, 9 aa, Artificial Sequence
  Description of Artificial Sequence Synthetic peptide
QQVGVVPYT

SEQ ID NO: 671 - protein, 7 aa, Artificial Sequence
  Description of Artificial Sequence Synthetic peptide
SSGQYWS

SEQ ID NO: 672 - protein, 12 aa, Artificial Sequence
  Description of Artificial Sequence Synthetic peptide
WIGEIYYSGSTR

SEQ ID NO: 673 - protein, 17 aa, Artificial Sequence
  Description of Artificial Sequence Synthetic peptide
ARDTPYYYDGGYYYYMD

SEQ ID NO: 674 - protein, 8 aa, Artificial Sequence
  Description of Artificial Sequence Synthetic peptide
SSSYLAWY

SEQ ID NO: 675 - protein, 10 aa, Artificial Sequence
  Description of Artificial Sequence Synthetic peptide
LLIYGASSRA

SEQ ID NO: 676 - protein, 8 aa, Artificial Sequence
  Description of Artificial Sequence Synthetic peptide
QQVGVVPY

SEQ ID NO: 677 - protein, 9 aa, Artificial Sequence
  Description of Artificial Sequence Synthetic peptide
GGSISSGQY

SEQ ID NO: 678 - protein, 7 aa, Artificial Sequence
  Description of Artificial Sequence Synthetic peptide
IYYSGST

SEQ ID NO: 679 - protein, 18 aa, Artificial Sequence
  Description of Artificial Sequence Synthetic peptide
ARDTPYYYDGGYYYYMDV

SEQ ID NO: 680 - protein, 7 aa, Artificial Sequence
  Description of Artificial Sequence Synthetic peptide
QSVSSSY

SEQ ID NO: 681 - protein, 3 aa, Artificial Sequence
  Description of Artificial Sequence Synthetic peptide
GAS

SEQ ID NO: 682 - protein, 9 aa, Artificial Sequence
  Description of Artificial Sequence Synthetic peptide
QQVGVVPYT

SEQ ID NO: 683 - protein, 125 aa, Artificial Sequence
  Description of Artificial Sequence Synthetic polypeptide
QVQLQESGPGLVKPSQTLSLTCTVSGGSISSGQYWSWIRQHPGKGLEWIGEIYYSGSTRYNPSLKSRVTISVDTSKDQFSLKLSSVTAADTAVYYCARDTPYYYDGGYYYYMDVWGKGTTVTVSS

SEQ ID NO: 684 - protein, 108 aa, Artificial Sequence; identical to SEQ ID NO: 456
  Description of Artificial Sequence Synthetic polypeptide

SEQ ID NO: 685 - protein, 11 aa, Artificial Sequence
  Description of Artificial Sequence Synthetic peptide
GGSISSGQYWS

SEQ ID NO: 686 - protein, 16 aa, Artificial Sequence
  Description of Artificial Sequence Synthetic peptide
EIYYSGSTRYNPSLKS

SEQ ID NO: 687 - protein, 16 aa, Artificial Sequence
  Description of Artificial Sequence Synthetic peptide
DTPYYYDGGYYYYMDV

SEQ ID NO: 688 - protein, 12 aa, Artificial Sequence
  Description of Artificial Sequence Synthetic peptide
RASQSVSSSYLA

SEQ ID NO: 689 - protein, 7 aa, Artificial Sequence
  Description of Artificial Sequence Synthetic peptide
GASSRAT

SEQ ID NO: 690 - protein, 9 aa, Artificial Sequence
  Description of Artificial Sequence Synthetic peptide
QQVGVVPYT

SEQ ID NO: 691 - protein, 6 aa, Artificial Sequence
  Description of Artificial Sequence Synthetic peptide
SGQYWS

SEQ ID NO: 692 - protein, 16 aa, Artificial Sequence
  Description of Artificial Sequence Synthetic peptide
EIYYSGSTRYNPSLKS

SEQ ID NO: 693 - protein, 16 aa, Artificial Sequence
  Description of Artificial Sequence Synthetic peptide
DTPYYYDGGYYYYMDV

SEQ ID NO: 694 - protein, 12 aa, Artificial Sequence
  Description of Artificial Sequence Synthetic peptide
RASQSVSSSYLA

SEQ ID NO: 695 - protein, 7 aa, Artificial Sequence
  Description of Artificial Sequence Synthetic peptide
GASSRAT

SEQ ID NO: 696 - protein, 9 aa, Artificial Sequence
  Description of Artificial Sequence Synthetic peptide
QQVGVVPYT

SEQ ID NO: 697 - protein, 8 aa, Artificial Sequence
  Description of Artificial Sequence Synthetic peptide
GGSISSGQ

SEQ ID NO: 698 - protein, 3 aa, Artificial Sequence
  Description of Artificial Sequence Synthetic peptide
YSG

SEQ ID NO: 699 - protein, 14 aa, Artificial Sequence
  Description of Artificial Sequence Synthetic peptide
TPYYYDGGYYYYMD

SEQ ID NO: 700 - protein, 8 aa, Artificial Sequence
  Description of Artificial Sequence Synthetic peptide
SQSVSSSY

SEQ ID NO: 701 - protein, 3 aa, Artificial Sequence
  Description of Artificial Sequence Synthetic peptide
GAS

SEQ ID NO: 702 - protein, 6 aa, Artificial Sequence
  Description of Artificial Sequence Synthetic peptide
VGVVPY

SEQ ID NO: 703 - protein, 11 aa, Artificial Sequence
  Description of Artificial Sequence Synthetic peptide
GGSISSGQYWS

SEQ ID NO: 704 - protein, 9 aa, Artificial Sequence
  Description of Artificial Sequence Synthetic peptide
EIYYSGSTR

SEQ ID NO: 705 - protein, 16 aa, Artificial Sequence
  Description of Artificial Sequence Synthetic peptide
DTPYYYDGGYYYYMDV

SEQ ID NO: 706 - protein, 12 aa, Artificial Sequence
  Description of Artificial Sequence Synthetic peptide
RASQSVSSSYLA

SEQ ID NO: 707 - protein, 7 aa, Artificial Sequence
  Description of Artificial Sequence Synthetic peptide
GASSRAT

SEQ ID NO: 708 - protein, 9 aa, Artificial Sequence
  Description of Artificial Sequence Synthetic peptide
QQVGVVPYT

SEQ ID NO: 709 - protein, 7 aa, Artificial Sequence
  Description of Artificial Sequence Synthetic peptide
SSGQYWS

SEQ ID NO: 710 - protein, 12 aa, Artificial Sequence
  Description of Artificial Sequence Synthetic peptide
WIGEIYYSGSTR

SEQ ID NO: 711 - protein, 17 aa, Artificial Sequence
  Description of Artificial Sequence Synthetic peptide
ARDTPYYYDGGYYYYMD

SEQ ID NO: 712 - protein, 8 aa, Artificial Sequence
  Description of Artificial Sequence Synthetic peptide
SSSYLAWY

SEQ ID NO: 713 - protein, 10 aa, Artificial Sequence
  Description of Artificial Sequence Synthetic peptide
LLIYGASSRA

SEQ ID NO: 714 - protein, 8 aa, Artificial Sequence
  Description of Artificial Sequence Synthetic peptide
QQVGVVPY

SEQ ID NO: 715 - protein, 9 aa, Artificial Sequence
  Description of Artificial Sequence Synthetic peptide
GGSISSGQY

SEQ ID NO: 716 - protein, 7 aa, Artificial Sequence
  Description of Artificial Sequence Synthetic peptide
IYYSGST

SEQ ID NO: 717 - protein, 18 aa, Artificial Sequence
  Description of Artificial Sequence Synthetic peptide
ARDTPYYYDGGYYYYMDV

SEQ ID NO: 718 - protein, 7 aa, Artificial Sequence
  Description of Artificial Sequence Synthetic peptide
QSVSSSY

SEQ ID NO: 719 - protein, 3 aa, Artificial Sequence
  Description of Artificial Sequence Synthetic peptide
GAS

SEQ ID NO: 720 - protein, 9 aa, Artificial Sequence
  Description of Artificial Sequence Synthetic peptide
QQVGVVPYT

SEQ ID NO: 721 - protein, 125 aa, Artificial Sequence
  Description of Artificial Sequence Synthetic polypeptide
QVQLQESGPGLVKPSQTLSLTCTVSGGSISSGQYWSWIRQHPGKGLEWIGEIYYSGSTRYNPSLKSRVTISVDTSKNQFSLKLSSVTAADTAVYYCARDTPYYYDGGYYYYMDVWGKGTTVTVSS

SEQ ID NO: 722 - protein, 108 aa, Artificial Sequence; identical to SEQ ID NO: 456
  Description of Artificial Sequence Synthetic polypeptide

SEQ ID NO: 723 - protein, 10 aa, Artificial Sequence
  Description of Artificial Sequence Synthetic peptide
GYTFANYYMH

SEQ ID NO: 724 - protein, 17 aa, Artificial Sequence
  Description of Artificial Sequence Synthetic peptide
IINPSGGITVYAQKFQG

SEQ ID NO: 725 - protein, 12 aa, Artificial Sequence
  Description of Artificial Sequence Synthetic peptide
GGSKVAALAFDI

SEQ ID NO: 726 - protein, 11 aa, Artificial Sequence
  Description of Artificial Sequence Synthetic peptide
QASQDISNSLN

SEQ ID NO: 727 - protein, 7 aa, Artificial Sequence
  Description of Artificial Sequence Synthetic peptide
DASNLET

SEQ ID NO: 728 - protein, 9 aa, Artificial Sequence
  Description of Artificial Sequence Synthetic peptide
QQYNFHPLT

SEQ ID NO: 729 - protein, 5 aa, Artificial Sequence
  Description of Artificial Sequence Synthetic peptide
NYYMH

SEQ ID NO: 730 - protein, 17 aa, Artificial Sequence
  Description of Artificial Sequence Synthetic peptide
IINPSGGITVYAQKFQG

SEQ ID NO: 731 - protein, 12 aa, Artificial Sequence
  Description of Artificial Sequence Synthetic peptide
GGSKVAALAFDI

SEQ ID NO: 732 - protein, 11 aa, Artificial Sequence
  Description of Artificial Sequence Synthetic peptide
QASQDISNSLN

SEQ ID NO: 733 - protein, 7 aa, Artificial Sequence
  Description of Artificial Sequence Synthetic peptide
DASNLET

SEQ ID NO: 734 - protein, 9 aa, Artificial Sequence
  Description of Artificial Sequence Synthetic peptide
QQYNFHPLT

SEQ ID NO: 735 - protein, 7 aa, Artificial Sequence
  Description of Artificial Sequence Synthetic peptide
GYTFANY

SEQ ID NO: 736 - protein, 4 aa, Artificial Sequence
  Description of Artificial Sequence Synthetic peptide
PSGG

SEQ ID NO: 737 - protein, 10 aa, Artificial Sequence
  Description of Artificial Sequence Synthetic peptide
GSKVAALAFD

SEQ ID NO: 738 - protein, 7 aa, Artificial Sequence
  Description of Artificial Sequence Synthetic peptide
SQDISNS

SEQ ID NO: 739 - protein, 3 aa, Artificial Sequence
  Description of Artificial Sequence Synthetic peptide
DAS

SEQ ID NO: 740 - protein, 6 aa, Artificial Sequence
  Description of Artificial Sequence Synthetic peptide
YNFHPL

SEQ ID NO: 741 - protein, 10 aa, Artificial Sequence
  Description of Artificial Sequence Synthetic peptide
GYTFANYYMH

SEQ ID NO: 742 - protein, 10 aa, Artificial Sequence
  Description of Artificial Sequence Synthetic peptide
IINPSGGITV

SEQ ID NO: 743 - protein, 12 aa, Artificial Sequence
  Description of Artificial Sequence Synthetic peptide
GGSKVAALAFDI

SEQ ID NO: 744 - protein, 11 aa, Artificial Sequence
  Description of Artificial Sequence Synthetic peptide
QASQDISNSLN

SEQ ID NO: 745 - protein, 7 aa, Artificial Sequence
  Description of Artificial Sequence Synthetic peptide
DASNLET

SEQ ID NO: 746 - protein, 9 aa, Artificial Sequence
  Description of Artificial Sequence Synthetic peptide
QQYNFHPLT

SEQ ID NO: 747 - protein, 6 aa, Artificial Sequence
  Description of Artificial Sequence Synthetic peptide
ANYYMH

SEQ ID NO: 748 - protein, 13 aa, Artificial Sequence
  Description of Artificial Sequence Synthetic peptide
WMGIINPSGGITV

SEQ ID NO: 749 - protein, 13 aa, Artificial Sequence
  Description of Artificial Sequence Synthetic peptide
ARGGSKVAALAFD

SEQ ID NO: 750 - protein, 7 aa, Artificial Sequence
  Description of Artificial Sequence Synthetic peptide
SNSLNWY

SEQ ID NO: 751 - protein, 10 aa, Artificial Sequence
  Description of Artificial Sequence Synthetic peptide
LLIYDASNLE

SEQ ID NO: 752 - protein, 8 aa, Artificial Sequence
  Description of Artificial Sequence Synthetic peptide
QQYNFHPL

SEQ ID NO: 753 - protein, 8 aa, Artificial Sequence
  Description of Artificial Sequence Synthetic peptide
GYTFANYY

SEQ ID NO: 754 - protein, 8 aa, Artificial Sequence
  Description of Artificial Sequence Synthetic peptide
INPSGGIT

SEQ ID NO: 755 - protein, 14 aa, Artificial Sequence
  Description of Artificial Sequence Synthetic peptide
ARGGSKVAALAFDI

SEQ ID NO: 756 - protein, 6 aa, Artificial Sequence
  Description of Artificial Sequence Synthetic peptide
QDISNS

SEQ ID NO: 757 - protein, 3 aa, Artificial Sequence
  Description of Artificial Sequence Synthetic peptide
DAS

SEQ ID NO: 758 - protein, 9 aa, Artificial Sequence
  Description of Artificial Sequence Synthetic peptide
QQYNFHPLT

SEQ ID NO: 759 - protein, 121 aa, Artificial Sequence
  Description of Artificial Sequence Synthetic polypeptide
QVQLVQSGAEVKKPGASVKVSCKASGYTFANYYMHWVRQAPGQGLEWMGIINPSGGITVYAQKFQGRVTMTRDTSTSTVYMELSSLRSEDTAVYYCARGGSKVAALAFDIWGQGTMVTVSS

SEQ ID NO: 760 - protein, 107 aa, Artificial Sequence
  Description of Artificial Sequence Synthetic polypeptide
DIQMTQSPSSLSASVGDRVTITCQASQDISNSLNWYQQKPGKAPKLLIYDASNLETGVPSRFSGSRSGTDFTFTISSLQPEDIATYYCQQYNFHPLTFGGGTKVEIK

SEQ ID NO: 761 - protein, 118 aa, Artificial Sequence
  Description of Artificial Sequence Synthetic polypeptide
  MOD_RES (31)..(31): D or S
  MOD_RES (35)..(35): A or G
  MOD_RES (50)..(50): A or T
EVQLLESGGGLVQPGGSLRLSCAASGFTFSXYAMXWVRQAPGKGLEWVSXISGSGGLTYYADSVKGRFTISRDNSKNTLYLQMNSLRAEDTAVYYCAKAPYGYYMDVWGKGTTVTVSS

SEQ ID NO: 762 - protein, 106 aa, Artificial Sequence; identical to SEQ ID NO: 38
  Description of Artificial Sequence Synthetic polypeptide

SEQ ID NO: 763 - protein, 123 aa, Artificial Sequence
  Description of Artificial Sequence Synthetic polypeptide
  MOD_RES (30)..(30): D or R
  MOD_RES (31)..(31): S, V, or A
  MOD_RES (51)..(51): I or V
  MOD_RES (55)..(55): S or N
  MOD_RES (106)..(106): F or Y
QVQLVQSGAEVKKPGASVKVSCKASGYTFXXYGISWVRQAPGQGLEWMGWXAPYXGNTNYAQKLQGRVTMTTDTSTSTAYMELRSLRSDDTAVYYCARDAGTYSPXGYGMDVWGQGTTVTVSS

SEQ ID NO: 764 - protein, 108 aa, Artificial Sequence
  Description of Artificial Sequence Synthetic polypeptide
  MOD_RES (24)..(24): R or Q
  MOD_RES (27)..(27): Q, E, or H
  MOD_RES (30)..(30): S, D, or N
  MOD_RES (31)..(31): S or N
  MOD_RES (50)..(50): K or S
  MOD_RES (52)..(52): S or Y
  MOD_RES (53)..(53): S, Y, or N
  MOD_RES (56)..(56): S or Y
  MOD_RES (90)..(90): Q, L, or R
  MOD_RES (93)..(93): S or K
DIQMTQSPSTLSASVGDRVTITCXASXSIXXWLAWYQQKPGKAPKLLIYXAXXLEXGVPSRFSGSGSGTEFTLTISSLQPDDFATYYCQXFQXLPPFTFGGGTKVEIK

SEQ ID NO: 765 - protein, 119 aa, Artificial Sequence
  Description of Artificial Sequence Synthetic polypeptide
  MOD_RES (30)..(30): H or R
  MOD_RES (32)..(32): R or Y
QVQLVESGGGVVQPGRSLRLSCAASGFTFXSXGMHWVRQAPGKGLEWVAVITYDGINKYYADSVEGRFTISRDNSKNTLYLQMNSLRAEDTAVYYCARDGVYYGVYDYWGQGTLVTVSS

SEQ ID NO: 766 - protein, 113 aa, Artificial Sequence; identical to SEQ ID NO: 342
  Description of Artificial Sequence Synthetic polypeptide

SEQ ID NO: 767 - protein, 124 aa, Artificial Sequence; identical to SEQ ID NO: 417
  Description of Artificial Sequence Synthetic polypeptide

SEQ ID NO: 768 - protein, 107 aa, Artificial Sequence; identical to SEQ ID NO: 418
  Description of Artificial Sequence Synthetic polypeptide

SEQ ID NO: 769 - protein, 125 aa, Artificial Sequence
  Description of Artificial Sequence Synthetic polypeptide
  MOD_RES (6)..(6): E or Q
  MOD_RES (7)..(7): S or W
  MOD_RES (9)..(9): P or A
  MOD_RES (12)..(12): V or L
  MOD_RES (16)..(16): Q or E
  MOD_RES (23)..(23): T or A
  MOD_RES (25)..(25): S or Y
  MOD_RES (29)..(29): I or L
  MOD_RES (33)..(33): Q or Y
  MOD_RES (41)..(41): H or P
  MOD_RES (53)..(53): Y or G
  MOD_RES (54)..(54): Y or A
  MOD_RES (77)..(77): N or D
  MOD_RES (100)..(100): T or A
  MOD_RES (105)..(105): E, G, or D
QVQLQXXGXGLXKPSXTLSLTCXVXGGSXSSGXYWSWIRQXPGKGLEWIGEIXXSGSTRYNPSLKSRVTISVDTSKXQFSLKLSSVTAADTAVYYCARDXPYYYXGGYYYYMDVWGKGTTVTVSS

SEQ ID NO: 770 - protein, 108 aa, Artificial Sequence
  Description of Artificial Sequence Synthetic polypeptide
  MOD_RES (27)..(27): Q, E, or D
  MOD_RES (30)..(30): S or D
  MOD_RES (33)..(33): Y or F
  MOD_RES (53)..(53): S, D, F, or Y
  MOD_RES (54)..(54): S or T
  MOD_RES (56)..(56): A or Q
  MOD_RES (57)..(57): T or N
  MOD_RES (92)..(92): V, A, or D
EIVLTQSPGTLSLSPGERATLSCRASXSVXSSXLAWYQQKPGQAPRLLIYGAXXRXXGIPDRFSGSGSGTDFTLTISRLEPEDFAVYYCQQXGVVPYTFGGGTKVEIK

SEQ ID NO: 771 - protein, 121 aa, Artificial Sequence; identical to SEQ ID NO: 759
  Description of Artificial Sequence Synthetic polypeptide

SEQ ID NO: 772 - protein, 107 aa, Artificial Sequence; identical to SEQ ID NO: 760
  Description of Artificial Sequence Synthetic polypeptide

SEQ ID NO: 773 - protein, 10 aa, Artificial Sequence
  Description of Artificial Sequence Synthetic peptide
  MOD_RES (6)..(6): D or S
  MOD_RES (10)..(10): A or G
GFTFSXYAMX

SEQ ID NO: 774 - protein, 17 aa, Artificial Sequence
  Description of Artificial Sequence Synthetic peptide
  MOD_RES (1)..(1): A or T
XISGSGGLTYYADSVKG

SEQ ID NO: 775 - protein, 9 aa, Artificial Sequence
  Description of Artificial Sequence Synthetic peptide
APYGYYMDV

SEQ ID NO: 776 - protein, 11 aa, Artificial Sequence
  Description of Artificial Sequence Synthetic peptide
RASQSISSWLA

SEQ ID NO: 777 - protein, 7 aa, Artificial Sequence
  Description of Artificial Sequence Synthetic peptide
KASSLES

SEQ ID NO: 778 - protein, 8 aa, Artificial Sequence
  Description of Artificial Sequence Synthetic peptide
QQYKSYIT

SEQ ID NO: 779 - protein, 10 aa, Artificial Sequence
  Description of Artificial Sequence Synthetic peptide
  MOD_RES (5)..(5): D or R
  MOD_RES (6)..(6): S or V
GYTFXXYGIS

SEQ ID NO: 780 - protein, 17 aa, Artificial Sequence
  Description of Artificial Sequence Synthetic peptide
  MOD_RES (2)..(2): I or V
  MOD_RES (6)..(6): S or N
WXAPYXGNTNYAQKLQG

SEQ ID NO: 781 - protein, 14 aa, Artificial Sequence
  Description of Artificial Sequence Synthetic peptide
  MOD_RES (8)..(8): F or Y
DAGTYSPXGYGMDV

SEQ ID NO: 782 - protein, 11 aa, Artificial Sequence
  Description of Artificial Sequence Synthetic peptide
  MOD_RES (1)..(1): R or Q
  MOD_RES (4)..(4): Q, E, or H
  MOD_RES (7)..(7): S, D, or N
  MOD_RES (8)..(8): S or N
XASXSIXXWLA

SEQ ID NO: 783 - protein, 7 aa, Artificial Sequence
  Description of Artificial Sequence Synthetic peptide
  MOD_RES (1)..(1): K or S
  MOD_RES (3)..(3): S or Y
  MOD_RES (4)..(4): S, Y, or N
  MOD_RES (7)..(7): S or Y
XAXXLEX

SEQ ID NO: 784 - protein, 10 aa, Artificial Sequence
  Description of Artificial Sequence Synthetic peptide
  MOD_RES (2)..(2): Q, L, or R
  MOD_RES (5)..(5): S or K
QXFQXLPPFT

SEQ ID NO: 785 - protein, 10 aa, Artificial Sequence
  Description of Artificial Sequence Synthetic peptide
  MOD_RES (5)..(5): H or R
  MOD_RES (7)..(7): R or Y
GFTFXSXGMH

SEQ ID NO: 786 - protein, 17 aa, Artificial Sequence
  Description of Artificial Sequence Synthetic peptide
VITYDGINKYYADSVEG

SEQ ID NO: 787 - protein, 10 aa, Artificial Sequence
  Description of Artificial Sequence Synthetic peptide
DGVYYGVYDY

SEQ ID NO: 788 - protein, 17 aa, Artificial Sequence
  Description of Artificial Sequence Synthetic peptide
KSSQSVLFSSNNKNYLA

SEQ ID NO: 789 - protein, 7 aa, Artificial Sequence
  Description of Artificial Sequence Synthetic peptide
WASTRES

SEQ ID NO: 790 - protein, 9 aa, Artificial Sequence
  Description of Artificial Sequence Synthetic peptide
QQFHSYPLT

SEQ ID NO: 791 - protein, 10 aa, Artificial Sequence
  Description of Artificial Sequence Synthetic peptide
GGTFSSNAIG

SEQ ID NO: 792 - protein, 17 aa, Artificial Sequence
  Description of Artificial Sequence Synthetic peptide
SIIPIIGFANYAQKFQG

SEQ ID NO: 793 - protein, 15 aa, Artificial Sequence
  Description of Artificial Sequence Synthetic peptide
DSGYYYGASSFGMDV

SEQ ID NO: 794 - protein, 11 aa, Artificial Sequence
  Description of Artificial Sequence Synthetic peptide
RASQSVSSNLA

SEQ ID NO: 795 - protein, 7 aa, Artificial Sequence
  Description of Artificial Sequence Synthetic peptide
GASTRAT

SEQ ID NO: 796 - protein, 9 aa, Artificial Sequence
  Description of Artificial Sequence Synthetic peptide
EQYNNLPLT

SEQ ID NO: 797 - protein, 11 aa, Artificial Sequence
  Description of Artificial Sequence Synthetic peptide
  MOD_RES (4)..(4): I or L
  MOD_RES (8)..(8): Q or Y
GGSXSSGXYWS

SEQ ID NO: 798 - protein, 16 aa, Artificial Sequence
  Description of Artificial Sequence Synthetic peptide
  MOD_RES (3)..(3): Y or G
  MOD_RES (4)..(4): Y or A
EIXXSGSTRYNPSLKS

SEQ ID NO: 799 - protein, 16 aa, Artificial Sequence
  Description of Artificial Sequence Synthetic peptide
  MOD_RES (2)..(2): T or A
  MOD_RES (7)..(7): E, G, or D
DXPYYYXGGYYYYMDV

SEQ ID NO: 800 - protein, 12 aa, Artificial Sequence
  Description of Artificial Sequence Synthetic peptide
  MOD_RES (4)..(4): Q, E, or D
  MOD_RES (7)..(7): S or D
  MOD_RES (10)..(10): Y or F
RASXSVXSSXLA

SEQ ID NO: 801 - protein, 7 aa, Artificial Sequence
  Description of Artificial Sequence Synthetic peptide
  MOD_RES (3)..(3): S, D, F, or Y
  MOD_RES (4)..(4): S or T
  MOD_RES (6)..(6): A or Q
  MOD_RES (7)..(7): T or N
GAXXRXX

SEQ ID NO: 802 - protein, 9 aa, Artificial Sequence
  Description of Artificial Sequence Synthetic peptide
  MOD_RES (3)..(3): V, A, or D
QQXGVVPYT

SEQ ID NO: 803 - protein, 10 aa, Artificial Sequence
  Description of Artificial Sequence Synthetic peptide
GYTFANYYMH

SEQ ID NO: 804 - protein, 17 aa, Artificial Sequence
  Description of Artificial Sequence Synthetic peptide
IINPSGGITVYAQKFQG

SEQ ID NO: 805 - protein, 12 aa, Artificial Sequence
  Description of Artificial Sequence Synthetic peptide
GGSKVAALAFDI

SEQ ID NO: 806 - protein, 11 aa, Artificial Sequence
  Description of Artificial Sequence Synthetic peptide
QASQDISNSLN

SEQ ID NO: 807 - protein, 7 aa, Artificial Sequence
  Description of Artificial Sequence Synthetic peptide
DASNLET

SEQ ID NO: 808 - protein, 9 aa, Artificial Sequence
  Description of Artificial Sequence Synthetic peptide
QQYNFHPLT

SEQ ID NO: 809 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 810 - protein, 219 aa, Homo sapiens
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 811 - protein, 227 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRETGHHHHHH

SEQ ID NO: 812 - protein, 455 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRETGENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 813 - protein, 296 aa, Macaca fascicularis
METPAWPRVPRPETAVARTLLLGWVFAQVAGASGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESREIFYIIGAVVFVVIILVIILAISLHKCKKARVGRSWKENSPLNVA

SEQ ID NO: 814 - protein, 220 aa, Macaca fascicularis
SGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESRE

SEQ ID NO: 815 - protein, 228 aa, Artificial Sequence
  Description of Artificial Sequence Synthetic polypeptide
SGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESRETGHHHHHH

SEQ ID NO: 816 - protein, 456 aa, Artificial Sequence
  Description of Artificial Sequence Synthetic polypeptide
SGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESRETGENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 817 - protein, 294 aa, Mus musculus
MAILVRPRLLAALAPTFLGCLLLQVTAGAGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGETLIIVGAVVLLATIFIILLSISLCKRRKNRAGQKGKNTPSRLA

SEQ ID NO: 818 - protein, 223 aa, Mus musculus
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGE

SEQ ID NO: 819 - protein, 231 aa, Artificial Sequence
  Description of Artificial Sequence Synthetic polypeptide
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGETGHHHHHH

SEQ ID NO: 820 - protein, 459 aa, Artificial Sequence
  Description of Artificial Sequence Synthetic polypeptide
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGETGENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 821 - protein, 120 aa, Artificial Sequence
  Description of Artificial Sequence Synthetic polypeptide
EVQLVQSGAEVKKPGESLRISCKGSGYTFAPYWIEWVRQMPGKGLEWMGDILPGTGFTTYSPSFQGHVTISADKSISTAYLQWSSLKASDTAMYYCARSGYYGNSGFAYWGQGTLVTVSS

SEQ ID NO: 822 - protein, 113 aa, Artificial Sequence
  Description of Artificial Sequence Synthetic polypeptide
DIVMTQTPLSLPVTPGEPASISCKSSQSLLSSGNQKNYLTWYLQKPGQSPQLLIYWASTRESGVPDRFSGSGSGTDFTLKISRVEAEDVGVYYCQNDYTYPLTFGQGTKLEIK

SEQ ID NO: 823 - protein, 5 aa, Artificial Sequence
  Description of Artificial Sequence Synthetic peptide
  See specification as filed for detailed description of substitutions and preferred embodiments
GGGGS

SEQ ID NO: 824 - protein, 292 aa, Sus scrofa
MATPTGPPVSCPKAAVARALLLGWVLVQVAGATGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRELFLIVGAVVFAVIVFVLVLSVSLYKCRKERAGPSGKENAPLNVA

SEQ ID NO: 825 - protein, 216 aa, Sus scrofa
TGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRE

SEQ ID NO: 826 - protein, 224 aa, Artificial Sequence
  Description of Artificial Sequence Synthetic polypeptide
TGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRETGHHHHHH

SEQ ID NO: 827 - protein, 452 aa, Artificial Sequence
  Description of Artificial Sequence Synthetic polypeptide
TGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRETGENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 828 - protein, 118 aa, Artificial Sequence
  Description of Artificial Sequence Synthetic polypeptide
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 829 - protein, 107 aa, Artificial Sequence
  Description of Artificial Sequence Synthetic polypeptide
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 830 - protein, 117 aa, Artificial Sequence
  Description of Artificial Sequence Synthetic polypeptide
QVQLVESGGGVVQPGRSLRLSCKASGFNIKDYYMHWVRQAPGKGLEWIGLIDPENGNTIYDPKFQGRFTISADNSKNTLFLQMDSLRPEDTAVYYCARDNSYYFDYWGQGTPVTVSS

SEQ ID NO: 831 - protein, 107 aa, Artificial Sequence
  Description of Artificial Sequence Synthetic polypeptide
DIQMTQSPSSLSASVGDRVTITCKASQDIRKYLNWYQQKPGKAPKLLIYYATSLADGVPSRFSGSGSGTDYTFTISSLQPEDIATYYCLQHGESPYTFGQGTKLEIT

SEQ ID NO: 832 - protein, 292 aa, Oryctolagus cuniculus
MAPPTRLQVPRPGTAVPYTVLLGWLLAQVARAADTTGRAYNLTWKSTNFKTILEWEPKSIDHVYTVQISTRLENWKSKCFLTAETECDLTDEVVKDVGQTYMARVLSYPARNGNTTGFPEEPPFRNSPEFTPYLDTNLGQPTIQSFEQVGTKLNVTVQDARTLVRRNGTFLSLRAVFGKDLNYTLYYWRASSTGKKTATTNTNEFLIDVDKGENYCFSVQAVIPSRKRKQRSPESLTECTSREQGRAREMFFIIGAVVVVALLIIVLSVTVYKCRKARAGPSGKESSPLNIA

SEQ ID NO: 833 - protein, 218 aa, Oryctolagus cuniculus
ADTTGRAYNLTWKSTNFKTILEWEPKSIDHVYTVQISTRLENWKSKCFLTAETECDLTDEVVKDVGQTYMARVLSYPARNGNTTGFPEEPPFRNSPEFTPYLDTNLGQPTIQSFEQVGTKLNVTVQDARTLVRRNGTFLSLRAVFGKDLNYTLYYWRASSTGKKTATTNTNEFLIDVDKGENYCFSVQAVIPSRKRKQRSPESLTECTSREQGRAREM

SEQ ID NO: 834 - protein, 226 aa, Artificial Sequence
  Description of Artificial Sequence Synthetic polypeptide
ADTTGRAYNLTWKSTNFKTILEWEPKSIDHVYTVQISTRLENWKSKCFLTAETECDLTDEVVKDVGQTYMARVLSYPARNGNTTGFPEEPPFRNSPEFTPYLDTNLGQPTIQSFEQVGTKLNVTVQDARTLVRRNGTFLSLRAVFGKDLNYTLYYWRASSTGKKTATTNTNEFLIDVDKGENYCFSVQAVIPSRKRKQRSPESLTECTSREQGRAREMTGHHHHHH

SEQ ID NO: 835 - protein, 452 aa, Artificial Sequence
  Description of Artificial Sequence Synthetic polypeptide
ADTTGRAYNLTWKSTNFKTILEWEPKSIDHVYTVQISTRLENWKSKCFLTAETECDLTDEVVKDVGQTYMARVLSYPARNGNTTGFPEEPPFRNSPEFTPYLDTNLGQPTIQSFEQVGTKLNVTVQDARTLVRRNGTFLSLRAVFGKDLNYTLYYWRASSTGKKTATTNTNEFLIDVDKGENYCFSVQAVIPSRKRKQRSPESLTECTSREQGRAREMENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 836 - protein, 123 aa, Artificial Sequence
  Description of Artificial Sequence Synthetic polypeptide
QVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSS

SEQ ID NO: 837 - protein, 108 aa, Artificial Sequence; identical to SEQ ID NO: 190
  Description of Artificial Sequence Synthetic polypeptide

SEQ ID NO: 838 - protein, 224 aa, Rattus sp.
AGTPPGKAFNLTWISTDFKTILEWQPKPTNYTYTVQISDRSRNWKYKCTGTTDTECDLTDEIVKDVNWTYEARVLSVPWRNSTHGKETLFGTHGEEPPFTNARKFLPYRDTKIGQPVIQKYEQGGTKLKVTVKDSFTLVRKNGTFLTLRQVFGNDLGYILTYRKDSSTGRKTNTTHTNEFLIDVEKGVSYCFFAQAVIFSRKTNHKSPESITKCTEQWKSVLGE

SEQ ID NO: 839 - protein, 224 aa, Artificial Sequence
  Description of Artificial Sequence Synthetic polypeptide
AGTPPGKAFNLTWISTDFKTILEWQPKPTNYTYTVQISDRSRNWKYKCTGTTDTECDLTDEIVKDVNWTYEARVLSVPWRNSTHGKETLFGTHGEEPPFTNARKFLPYRDTKLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 840 - protein, 218 aa, Artificial Sequence
  Description of Artificial Sequence Synthetic polypeptide
AGTPPGKAFNLTWISTDFKTILEWQPKPTNYTYTVQISDRSRNWKYKCTGTTDTECDLTDEIVKDVNWTYEARVLSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 841 - protein, 218 aa, Artificial Sequence
  Description of Artificial Sequence Synthetic polypeptide
AGTPPGKAFNLTWISTDFKTILEWQPKPTNYTYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 842 - protein, 219 aa, Artificial Sequence
  Description of Artificial Sequence Synthetic polypeptide
SGTTNTVAAYNLTWKSTNFKTILEWEPKPVNQVYTVQISDRSRNWKYKCTGTTDTECDLTDEIVKDVNWTYEARVLSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 843 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSVPWRNSTHGTHGEEPPFTNARKFLPYRDTKLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 844 - protein, 225 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSVPWRNSTHGKETLFGTHGEEPPFTNARKFLPYRDTKLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 845 - protein, 225 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSVPWRNSTHGKETLFGTHGEEPPYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 846 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLFTNARKFLPYRDTKLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 847 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNIGQPVIQKYEQGGTKLKVTVKDSFTLVRKNGTFLTLRQVFGNDLGYILTYRKDSSTGRKTNTTHTNEFLIDVEKGVSYCFFAQAVIFSRKTNHKSPESITKCTEQWKSVLGE

SEQ ID NO: 848 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNIGQPVIQKYEQGGTKLKVTVKDSFTLVRKNGTFLTLRQVFGNDLGYILTYRKSSSSGKKTAKTNTNEFLIDVDKGENYCFSVQAVIPSRTVNRKSTDSPVECMGQEKGEFRE

SEQ ID NO: 849 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNIGQPVIQKYEQGGTKLKVTVKDSFTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 850 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRKNGTFLTLRQVFGNDLGYILTYRKSSSSGKKTAKTNTNEFLIDVDKGENYCFSVQAVIPSRTVNRKSTDSPVECMGQEKGEFRE

SEQ ID NO: 851 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRKNGTFLTLRQVFGKDLIYTLYYWKSSSSGKKTAKTNTNEFLIDVDKGENYCFSVQAVIPSRTVNRKSTDSPVECMGQEKGEFRE

SEQ ID NO: 852 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRKNGTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 853 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLTLRQVFGKDLIYTLYYWKSSSSGKKTAKTNTNEFLIDVDKGENYCFSVQAVIPSRTVNRKSTDSPVECMGQEKGEFRE

SEQ ID NO: 854 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGNDLGYILTYRKSSSSGKKTAKTNTNEFLIDVDKGENYCFSVQAVIPSRTVNRKSTDSPVECMGQEKGEFRE

SEQ ID NO: 855 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGNDLIYTLYYWKSSSSGKKTAKTNTNEFLIDVDKGENYCFSVQAVIPSRTVNRKSTDSPVECMGQEKGEFRE

SEQ ID NO: 856 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLGYILTYRKSSSSGKKTAKTNTNEFLIDVDKGENYCFSVQAVIPSRTVNRKSTDSPVECMGQEKGEFRE

SEQ ID NO: 857 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKDSSTGRKTNTTHTNEFLIDVEKGVSYCFFAQAVIFSRKTNHKSPESITKCTEQWKSVLGE

SEQ ID NO: 858 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKDSSTGRKTNTTHTNEFLIDVEKGVSYCFFAQAVIPSRTVNRKSTDSPVECMGQEKGEFRE

SEQ ID NO: 859 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKDSSTGRKTNTTHTNEFLIDVDKGENYCFSVQAVIPSRTVNRKSTDSPVECMGQEKGEFRE

SEQ ID NO: 860 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKDSSTGRKTAKTNTNEFLIDVDKGENYCFSVQAVIPSRTVNRKSTDSPVECMGQEKGEFRE

SEQ ID NO: 861 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTNTTHTNEFLIDVDKGENYCFSVQAVIPSRTVNRKSTDSPVECMGQEKGEFRE

SEQ ID NO: 862 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVEKGVSYCFFAQAVIPSRTVNRKSTDSPVECMGQEKGEFRE

SEQ ID NO: 863 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIFSRKTNHKSPESITKCTEQWKSVLGE

SEQ ID NO: 864 - protein, 224 aa, Artificial Sequence
  Description of Artificial Sequence Synthetic polypeptide
AGTPPGKAFNLTWISTDFKTILEWQPKPTNYTYTVQISDRSRNWKYKCTGTTDTECDLTDEIVKDVNWTYEARVLSVPWRNSTHGKETLFGTHGEEPPFTNARKFLPYRDTKIGQPVIQKYEQGGTKLKVTVKDSFTLVRRNNTFLSLRDVFGKDLIYTLYYWKSSSSGKKTAKTNTNEFLIDVDKGENYCFSVQAVIFSRKTNHKSPESITKCTEQWKSVLGE

SEQ ID NO: 865 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVNQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 866 - protein, 219 aa, Artificial Sequence; identical to SEQ ID NO: 855
  Description of Artificial Sequence Synthetic polypeptide

SEQ ID NO: 867 - protein, 219 aa, Artificial Sequence
  Description of Artificial Sequence Synthetic polypeptide
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTHTNEFLIDVDKGENYCFSVQAVIPSRKVNRKSTDSPVECMGQEKGEFRE

SEQ ID NO: 868 - protein, 123 aa, Artificial Sequence
  Description of Artificial Sequence Synthetic polypeptide
  MOD_RES (31)..(31): V or A
  MOD_RES (55)..(55): N or S
QVQLVQSGAEVKKPGASVKVSCKASGYTFDXYGISWVRQAPGQGLEWMGWIAPYXGNTNYAQKLQGRVTMTTDTSTSTAYMELRSLRSDDTAVYYCARDAGTYSPFGYGMDVWGQGTTVTVSS

SEQ ID NO: 869 - protein, 108 aa, Artificial Sequence
  Description of Artificial Sequence Synthetic polypeptide
  MOD_RES (24)..(24): R or Q
  MOD_RES (27)..(27): Q or E
  MOD_RES (30)..(30): S or N
  MOD_RES (31)..(31): S or N
  MOD_RES (52)..(52): S or Y
  MOD_RES (53)..(53): S or N
  MOD_RES (56)..(56): S or Y
  MOD_RES (90)..(90): Q or L
  MOD_RES (93)..(93): S or K
DIQMTQSPSTLSASVGDRVTITCXASXSIXXWLAWYQQKPGKAPKLLIYKAXXLEXGVPSRFSGSGSGTEFTLTISSLQPDDFATYYCQXFQXLPPFTFGGGTKVEIK

SEQ ID NO: 870 - protein, 123 aa, Artificial Sequence
  Description of Artificial Sequence Synthetic polypeptide
  MOD_RES (55)..(55): N or S
QVQLVQSGAEVKKPGASVKVSCKASGYTFRSYGISWVRQAPGQGLEWMGWVAPYXGNTNYAQKLQGRVTMTTDTSTSTAYMELRSLRSDDTAVYYCARDAGTYSPYGYGMDVWGQGTTVTVSS

SEQ ID NO: 871 - protein, 108 aa, Artificial Sequence
  Description of Artificial Sequence Synthetic polypeptide
  MOD_RES (24)..(24): R or Q
  MOD_RES (27)..(27): Q or H
  MOD_RES (30)..(30): S or D
  MOD_RES (50)..(50): K or S
  MOD_RES (53)..(53): S or Y
  MOD_RES (90)..(90): Q, L, or R
DIQMTQSPSTLSASVGDRVTITCXASXSIXSWLAWYQQKPGKAPKLLIYXASXLESGVPSRFSGSGSGTEFTLTISSLQPDDFATYYCQXFQSLPPFTFGGGTKVEIK

SEQ ID NO: 872 - protein, 10 aa, Artificial Sequence
  Description of Artificial Sequence Synthetic peptide
  MOD_RES (6)..(6): V or A
GYTFDXYGIS

SEQ ID NO: 873 - protein, 17 aa, Artificial Sequence
  Description of Artificial Sequence Synthetic peptide
  MOD_RES (6)..(6): N or S
WIAPYXGNTNYAQKLQG

SEQ ID NO: 874 - protein, 14 aa, Artificial Sequence
  Description of Artificial Sequence Synthetic peptide
DAGTYSPFGYGMDV

SEQ ID NO: 875 - protein, 11 aa, Artificial Sequence
  Description of Artificial Sequence Synthetic peptide
  MOD_RES (1)..(1): R or Q
  MOD_RES (4)..(4): Q or E
  MOD_RES (7)..(7): S or N
  MOD_RES (8)..(8): S or N
XASXSIXXWLA

SEQ ID NO: 876 - protein, 7 aa, Artificial Sequence
  Description of Artificial Sequence Synthetic peptide
  MOD_RES (3)..(3): S or Y
  MOD_RES (4)..(4): S or N
  MOD_RES (7)..(7): S or Y
KAXXLEX

SEQ ID NO: 877 - protein, 10 aa, Artificial Sequence
  Description of Artificial Sequence Synthetic peptide
  MOD_RES (2)..(2): Q or L
  MOD_RES (5)..(5): S or K
QXFQXLPPFT

SEQ ID NO: 878 - protein, 10 aa, Artificial Sequence
  Description of Artificial Sequence Synthetic peptide
GYTFRSYGIS

SEQ ID NO: 879 - protein, 17 aa, Artificial Sequence
  Description of Artificial Sequence Synthetic peptide
  MOD_RES (6)..(6): S or N
WVAPYXGNTNYAQKLQG

SEQ ID NO: 880 - protein, 14 aa, Artificial Sequence
  Description of Artificial Sequence Synthetic peptide
DAGTYSPYGYGMDV

SEQ ID NO: 881 - protein, 11 aa, Artificial Sequence
  Description of Artificial Sequence Synthetic peptide
  MOD_RES (1)..(1): R or Q
  MOD_RES (4)..(4): Q or H
  MOD_RES (7)..(7): S or D
XASXSIXSWLA

SEQ ID NO: 882 - protein, 7 aa, Artificial Sequence
  Description of Artificial Sequence Synthetic peptide
  MOD_RES (1)..(1): K or S
  MOD_RES (4)..(4): S or Y
XASXLES

SEQ ID NO: 883 - protein, 10 aa, Artificial Sequence
  Description of Artificial Sequence Synthetic peptide
  MOD_RES (2)..(2): Q, L, or R
QXFQSLPPFT

SEQ ID NO: 884 - protein, 10 aa, Artificial Sequence
  Description of Artificial Sequence Synthetic peptide
GYTFDAYGIS

SEQ ID NO: 885 - protein, 17 aa, Artificial Sequence
  Description of Artificial Sequence Synthetic peptide
WIAPYSGNTNYAQKLQG

SEQ ID NO: 886 - protein, 14 aa, Artificial Sequence
  Description of Artificial Sequence Synthetic peptide
DAGTYSPFGYGMDV

SEQ ID NO: 887 - protein, 11 aa, Artificial Sequence
  Description of Artificial Sequence Synthetic peptide
RASESISNWLA

SEQ ID NO: 888 - protein, 7 aa, Artificial Sequence
  Description of Artificial Sequence Synthetic peptide
KAYSLEY

SEQ ID NO: 889 - protein, 10 aa, Artificial Sequence
  Description of Artificial Sequence Synthetic peptide
QQFQKLPPFT

SEQ ID NO: 890 - protein, 5 aa, Artificial Sequence
  Description of Artificial Sequence Synthetic peptide
AYGIS

SEQ ID NO: 891 - protein, 17 aa, Artificial Sequence
  Description of Artificial Sequence Synthetic peptide
WIAPYSGNTNYAQKLQG

SEQ ID NO: 892 - protein, 14 aa, Artificial Sequence
  Description of Artificial Sequence Synthetic peptide
DAGTYSPFGYGMDV

SEQ ID NO: 893 - protein, 11 aa, Artificial Sequence
  Description of Artificial Sequence Synthetic peptide
RASESISNWLA

SEQ ID NO: 894 - protein, 7 aa, Artificial Sequence
  Description of Artificial Sequence Synthetic peptide
KAYSLEY

SEQ ID NO: 895 - protein, 10 aa, Artificial Sequence
  Description of Artificial Sequence Synthetic peptide
QQFQKLPPFT

SEQ ID NO: 896 - protein, 7 aa, Artificial Sequence
  Description of Artificial Sequence Synthetic peptide
GYTFDAY

SEQ ID NO: 897 - protein, 4 aa, Artificial Sequence
  Description of Artificial Sequence Synthetic peptide
PYSG

SEQ ID NO: 898 - protein, 12 aa, Artificial Sequence
  Description of Artificial Sequence Synthetic peptide
AGTYSPFGYGMD

SEQ ID NO: 899 - protein, 7 aa, Artificial Sequence
  Description of Artificial Sequence Synthetic peptide
SESISNW

SEQ ID NO: 900 - protein, 3 aa, Artificial Sequence
  Description of Artificial Sequence Synthetic peptide
KAY

SEQ ID NO: 901 - protein, 7 aa, Artificial Sequence
  Description of Artificial Sequence Synthetic peptide
FQKLPPF

SEQ ID NO: 902 - protein, 10 aa, Artificial Sequence
  Description of Artificial Sequence Synthetic peptide
GYTFDAYGIS

SEQ ID NO: 903 - protein, 10 aa, Artificial Sequence
  Description of Artificial Sequence Synthetic peptide
WIAPYSGNTN

SEQ ID NO: 904 - protein, 14 aa, Artificial Sequence
  Description of Artificial Sequence Synthetic peptide
DAGTYSPFGYGMDV

SEQ ID NO: 905 - protein, 11 aa, Artificial Sequence
  Description of Artificial Sequence Synthetic peptide
RASESISNWLA

SEQ ID NO: 906 - protein, 7 aa, Artificial Sequence
  Description of Artificial Sequence Synthetic peptide
KAYSLEY

SEQ ID NO: 907 - protein, 10 aa, Artificial Sequence
  Description of Artificial Sequence Synthetic peptide
QQFQKLPPFT

SEQ ID NO: 908 - protein, 6 aa, Artificial Sequence
  Description of Artificial Sequence Synthetic peptide
DAYGIS

SEQ ID NO: 909 - protein, 13 aa, Artificial Sequence
  Description of Artificial Sequence Synthetic peptide
WMGWIAPYSGNTN

SEQ ID NO: 910 - protein, 15 aa, Artificial Sequence
  Description of Artificial Sequence Synthetic peptide
ARDAGTYSPFGYGMD

SEQ ID NO: 911 - protein, 7 aa, Artificial Sequence
  Description of Artificial Sequence Synthetic peptide
SNWLAWY

SEQ ID NO: 912 - protein, 10 aa, Artificial Sequence
  Description of Artificial Sequence Synthetic peptide
LLIYKAYSLE

SEQ ID NO: 913 - protein, 9 aa, Artificial Sequence
  Description of Artificial Sequence Synthetic peptide
QQFQKLPPF

SEQ ID NO: 914 - protein, 8 aa, Artificial Sequence
  Description of Artificial Sequence Synthetic peptide
GYTFDAYG

SEQ ID NO: 915 - protein, 8 aa, Artificial Sequence
  Description of Artificial Sequence Synthetic peptide
IAPYSGNT

SEQ ID NO: 916 - protein, 16 aa, Artificial Sequence
  Description of Artificial Sequence Synthetic peptide
ARDAGTYSPFGTGMDV

SEQ ID NO: 917 - protein, 6 aa, Artificial Sequence
  Description of Artificial Sequence Synthetic peptide
ESISNW

SEQ ID NO: 918 - protein, 3 aa, Artificial Sequence
  Description of Artificial Sequence Synthetic peptide
KAY

SEQ ID NO: 919 - protein, 10 aa, Artificial Sequence
  Description of Artificial Sequence Synthetic peptide
QQFQKLPPFT

SEQ ID NO: 920 - protein, 452 aa, Artificial Sequence
  Description of Artificial Sequence Synthetic polypeptide
QVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 921 - protein, 215 aa, Artificial Sequence
  Description of Artificial Sequence Synthetic polypeptide
DIQMTQSPSTLSASVGDRVTITCRASESISNWLAWYQQKPGKAPKLLIYKAYSLEYGVPSRFSGSGSGTEFTLTISSLQPDDFATYYCQQFQKLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 922 - protein, 452 aa, Artificial Sequence
  Description of Artificial Sequence Synthetic polypeptide
QVQLVQSGAEVKKPGASVKVSCKASGYTFRSYGISWVRQAPGQGLEWMGWVAPYSGNTNYAQKLQGRVTMTTDTSTSTAYMELRSLRSDDTAVYYCARDAGTYSPYGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 923 - protein, 215 aa, Artificial Sequence
  Description of Artificial Sequence Synthetic polypeptide
DIQMTQSPSTLSASVGDRVTITCQASQSIDSWLAWYQQKPGKAPKLLIYSASYLESGVPSRFSGSGSGTEFTLTISSLQPDDFATYYCQRFQSLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 924 - protein, 453 aa, Artificial Sequence
  Description of Artificial Sequence Synthetic polypeptide
QVQLQQWGAGLLKPSETLSLTCAVYGGSLSGYYWSWIRQPPGKGLEWIGEIGASGSTRYNPSLKSRVTISVDTSKNQFSLKLSSVTAADTAVYYCARDTPYYYEGGYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 925 - protein, 215 aa, Artificial Sequence
  Description of Artificial Sequence Synthetic polypeptide
EIVLTQSPGTLSLSPGERATLSCRASDSVDSSYLAWYQQKPGQAPRLLIYGAFSRANGIPDRFSGSGSGTDFTLTISRLEPEDFAVYYCQQAGVVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 926 - protein, 453 aa, Artificial Sequence; identical to SEQ ID NO: 924
  Description of Artificial Sequence Synthetic polypeptide

SEQ ID NO: 927 - protein, 215 aa, Artificial Sequence
  Description of Artificial Sequence Synthetic polypeptide
EIVLTQSPGTLSLSPGERATLSCRASQSVSSSFLAWYQQKPGQAPRLLIYGAYSRATGIPDRFSGSGSGTDFTLTISRLEPEDFAVYYCQQAGVVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 928 - protein, 4 aa, Homo sapiens
ELLG

SEQ ID NO: 929 - protein, 4 aa, Homo sapiens
EFLG

SEQ ID NO: 930 - protein, 214 aa, Artificial Sequence [sequence omitted]

SEQ ID NO: 931 - protein, 448 aa, Artificial Sequence
  Description of Artificial Sequence Synthetic polypeptide
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 932 - protein, 214 aa, Artificial Sequence
  Description of Artificial Sequence Synthetic polypeptide
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 933 - protein, 446 aa, Artificial Sequence
  Description of Artificial Sequence Synthetic polypeptide
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 934 - protein, 214 aa, Artificial Sequence
  Description of Artificial Sequence Synthetic polypeptide
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 935 - protein, 229 aa, Artificial Sequence
  Description of Artificial Sequence Synthetic polypeptide
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 936 - protein, 452 aa, Artificial Sequence
  Description of Artificial Sequence Synthetic polypeptide
QVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 937 - protein, 214 aa, Artificial Sequence
  Description of Artificial Sequence Synthetic polypeptide
DIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 938 - protein, 880 aa, Artificial Sequence
  Description of Artificial Sequence Synthetic polypeptide
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 939 - protein, 452 aa, Artificial Sequence
  Description of Artificial Sequence Synthetic polypeptide
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 940 - protein, 452 aa, Artificial Sequence; identical to SEQ ID NO: 939
  Description of Artificial Sequence Synthetic polypeptide

SEQ ID NO: 941 - protein, 452 aa, Artificial Sequence; identical to SEQ ID NO: 939
  Description of Artificial Sequence Synthetic polypeptide

SEQ ID NO: 942 - protein, 452 aa, Artificial Sequence
  Description of Artificial Sequence Synthetic polypeptide
QVQLVQSGAEVKKPGASVKVSCKASGYTFRSYGISWVRQAPGQGLEWMGWVAPYNGNTNYAQKLQGRVTMTTDTSTSTAYMELRSLRSDDTAVYYCARDAGTYSPYGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 943 - protein, 452 aa, Artificial Sequence
  Description of Artificial Sequence Synthetic polypeptide
  MOD_RES (31)..(31): V or A
  MOD_RES (55)..(55): N or S
QVQLVQSGAEVKKPGASVKVSCKASGYTFDXYGISWVRQAPGQGLEWMGWIAPYXGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 944 - protein, 215 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQFQSLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 945 - protein, 215 aa, Artificial Sequence
  Description of Artificial Sequence Synthetic polypeptide
DIQMTQSPSTLSASVGDRVTITCQASQSINNWLAWYQQKPGKAPKLLIYKAYNLESGVPSRFSGSGSGTEFTLTISSLQPDDFATYYCQLFQSLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 946 - protein, 215 aa, Artificial Sequence; identical to SEQ ID NO: 944
  Description of Artificial Sequence Synthetic polypeptide

SEQ ID NO: 947 - protein, 215 aa, Artificial Sequence
  Description of Artificial Sequence Synthetic polypeptide
DIQMTQSPSTLSASVGDRVTITCRASHSIDSWLAWYQQKPGKAPKLLIYKASYLESGVPSRFSGSGSGTEFTLTISSLQPDDFATYYCQLFQSLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 948 - protein, 215 aa, Artificial Sequence
  Description of Artificial Sequence Synthetic polypeptide
  MOD_RES (24)..(24): R or Q
  MOD_RES (27)..(27): Q or E
  MOD_RES (30)..(30): S or N
  MOD_RES (31)..(31): S or N
  MOD_RES (52)..(52): S or Y
  MOD_RES (53)..(53): S or N
  MOD_RES (56)..(56): S or Y
  MOD_RES (90)..(90): Q or L
  MOD_RES (93)..(93): S or K
DIQMTQSPSTLSASVGDRVTITCXASXSIXXWLAWYQQKPGKAPKLLIYKAXXLEXGVPSRFSGSGSGTEFTLTISSLQPDDFATYYCQXFQXLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0166765 A1
(43) Pub. Date: May 23, 2024

(54) INFLAMMATORY DISEASE TREATMENT USING ANTI-TISSUE FACTOR ANTIBODIES

(71) Applicant: Iconic Therapeutics LLC, Palo Alto, CA (US)

(72) Inventors: Thi-Sau MIGONE, Palo Alto, CA (US); William Greene, Palo Alto, CA (US)

(21) Appl. No.: 18/015,532

(22) PCT Filed: Jul. 10, 2021
(86) PCT No.: PCT/US2021/041192

§ 371 Date: Jan. 10, 2023

Related U.S. Application Data
(60) Provisional application No. 63/050,629, filed on Jul. 10, 2020.

(51) Int. Cl. C07K 16/36; A61K 39/395; A61K 45/06; A61P 29/00

(52) U.S. Cl. CPC C07K 16/36; A61K 39/3955; A61K 45/06; A61P 29/00; A61K 2039/545; C07K 2317/21

(57) ABSTRACT

Provided herein are antibodies that specifically bind to human tissue factor (TF), anti-TF antibody-drug conjugates (ADCs), and compositions comprising the antibodies or ADCs for treatment of inflammatory diseases. Also provided herein are methods of treating subjects having inflammatory diseases by administering the anti-TF antibodies or ADCs.

### CROSS-REFERENCE TO RELATED APPLICATION

This application claims priority to and the benefit of U.S. Provisional Patent Application No. 63/050,629, filed on Jul. 10, 2020, the entire contents of which are incorporated by reference herein for all purposes.

### SEQUENCE LISTING

The instant application contains a Sequence Listing which has been submitted electronically in ASCII format and is hereby incorporated by reference in its entirety. Said ASCII copy, created on Jul. 8, 2021, is named ITI-005WO_SL.txt and is 466,223 bytes in size.

### BACKGROUND

Blood coagulation involves a complex set of processes that result in blood clotting. Tissue factor (TF) plays an important role in these coagulation processes. TF is a cell surface receptor. The TF/FVIIa complex catalyzes conversion of the inactive protease factor X (FX) into the active protease factor Xa (FXa). FXa and its co-factor FVa form the prothrombinase complex, which generates thrombin from prothrombin. Thrombin converts soluble fibrinogen into insoluble strands of fibrin and catalyzes many other coagulation-related processes.

Inflammatory diseases include a vast array of disorders and conditions that are characterized by inflammation (local or systemic). During inflammation, there is a change in vascular dynamics and recruitment of innate and adaptive immune cells to the site of injury or disease. Inflammation is necessary for guarding the body against foreign bodies and is necessary for wound repair; however, in autoimmune and/or inflammatory diseases, the immune system triggers an inflammatory response in the absence of a foreign substance to fight, and the body's normal protective immune system mistakenly attacks itself, thereby affecting its own tissue. Inflammatory diseases continue to be a burden to patients because of life-long debilitating illness, increased mortality and high costs for therapy and care.

TF is thought to play a role in diseases characterized by local and systemic inflammation, but to date there are no approved anti-TF antibodies indicated for the treatment of inflammatory diseases. Aspects of the anti-TF antibodies, anti-TF antibody-drug conjugates (ADCs) and methods comprising use of the anti-TF antibodies and ADCs of this disclosure are described in international PCT application PCT/US2019/012427, U.S. utility application Ser. No. 16/959,652, and U.S. provisional application Nos. 62/713,797; 62/713,804; 62/646,788; 62/613,545; and 62/613,564, incorporated herein by reference in their entirety for all purposes.

### SUMMARY

Provided herein are antibodies that specifically bind human Tissue Factor (TF), anti-TF antibody-drug conjugates, and related methods. Provided herein are methods for treating inflammatory diseases by administering an antibody or ADC of the present disclosure.

In one aspect, provided herein is a method of treating an inflammatory disease in a subject in need thereof comprising administering to the subject an isolated antibody wherein the antibody binds to the extracellular domain of human Tissue Factor (TF), wherein the antibody binds human TF at a human TF binding site that is distinct from a human TF binding site bound by human FVIIa.

In some embodiments, the viral infection is severe acute respiratory syndrome coronavirus 2 (SARS-CoV-2). In some embodiments, the inflammatory disease is selected from: colitis, inflammatory bowel disease, arthritis, acute lung injury, acute respiratory distress syndrome (ARDS), and Respiratory Syncytial Virus (RSV). In some embodiments, the inflammatory disease is colitis. In some embodiments, the inflammatory disease is inflammatory bowel disease (IBD). In some embodiments, the inflammatory disease is arthritis. In some embodiments, the inflammatory disease is acute lung injury. In some embodiments, the inflammatory disease is ARDS. In some embodiments, the inflammatory disease is RSV. In some embodiments, the inflammatory disease is a cardiovascular disease or injury. In some embodiments, the cardiac disease or injury is myocardial infarction. In some embodiments, the inflammatory disease is a cardiovascular disease associated with upregulation of protease-activated receptor 2 (PAR-2).

In some embodiments, the antibody does not inhibit human thrombin generation as determined by thrombin generation assay (TGA). In some embodiments, the isolated human antibody does not inhibit or inhibits human thrombin generation to a lesser extent, as determined by thrombin generation assay (TGA), compared to a reference antibody comprising a V_{H} sequence of SEQ ID NO:821 and a V_{L} sequence of SEQ ID NO:822. In some embodiments, binding between the isolated antibody and a variant TF extracellular domain comprising a mutation at amino acid residue 149 of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay. In some embodiments, the antibody comprises all three heavy chain Complementary Determining Regions (CDRs) and all three light chain CDRs from an antibody group in Table 35, wherein the all three heavy chain CDRs and the all three light chain CDRs are from the same antibody group.

In some embodiments, the antibody comprises all three heavy chain Complementary Determining Regions (CDRs) and all three light chain CDRs from an antibody in any one of Tables 15-34, wherein the all three heavy chain CDRs and the all three light chain CDRs are from the same antibody. In some embodiments, the antibody comprises all three heavy chain CDRs and all three light chain CDRs from: the antibody designated 25A, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea. In some embodiments, the antibody comprises all three heavy chain CDRs and all three light chain CDRs from: the antibody designated 43B, the antibody designated 43B1, the antibody designated 43B7, the antibody designated 43D, the antibody designated 43D7, the antibody designated 43D8, the antibody designated 43E, or the antibody designated 43Ea. In some embodiments, the antibody comprises all three heavy chain CDRs and all three light chain CDRs from: the antibody designated 25A, the antibody designated 25A5, the antibody designated 25A5-T, the antibody designated 25G, the antibody designated 25G1, or the antibody designated 25G9.

In some embodiments, the antibody comprises a VH Domain sequence and VL domain sequence from Table 14, wherein the VH and VL domain sequences are from the same group in Table 14. In some embodiments, the antibody comprises a VH Domain sequence and VL domain sequence from Table 13, wherein the VH and VL domain sequences are from the same clone in Table 13.

In some embodiments, the antibody comprises: a VH-CDR1 comprising the sequence set forth in SEQ ID NO:797; a VH-CDR2 comprising the sequence set forth in SEQ ID NO:798; a VH-CDR3 comprising the sequence set forth in SEQ ID NO:799; a VL-CDR1 comprising the sequence set forth in SEQ ID NO:800; a VL-CDR2 comprising the sequence set forth in SEQ ID NO:801; and a VL-CDR3 comprising the sequence set forth in SEQ ID NO:802.

In some embodiments, the antibody comprises: a VH-CDR1 comprising the sequence set forth in SEQ ID NO:571; a VH-CDR2 comprising the sequence set forth in SEQ ID NO:572; a VH-CDR3 comprising the sequence set forth in SEQ ID NO:573; a VL-CDR1 comprising the sequence set forth in SEQ ID NO:574; a VL-CDR2 comprising the sequence set forth in SEQ ID NO:575; and a VL-CDR3 comprising the sequence set forth in SEQ ID NO:576.

In some embodiments, the antibody comprises: a VH-CDR1 comprising the sequence set forth in SEQ ID NO:609; a VH-CDR2 comprising the sequence set forth in SEQ ID NO:610; a VH-CDR3 comprising the sequence set forth in SEQ ID NO:611; a VL-CDR1 comprising the sequence set forth in SEQ ID NO:612; a VL-CDR2 comprising the sequence set forth in SEQ ID NO:613; and a VL-CDR3 comprising the sequence set forth in SEQ ID NO:614.

In some embodiments, the antibody comprises: a VH sequence comprising the sequence set forth in SEQ ID NO:769 and a VL sequence comprising the sequence set forth in SEQ ID NO:770. In some embodiments, the antibody comprises: a VH sequence comprising the sequence set forth in SEQ ID NO:569 and a VL sequence comprising the sequence set forth in SEQ ID NO:570. In some embodiments, the antibody comprises: a VH sequence comprising the sequence set forth in SEQ ID NO:607 and a VL sequence comprising the sequence set forth in SEQ ID NO:608. In some embodiments, the antibody comprises: a heavy chain comprising the sequence set forth in SEQ ID NO:924 and a light chain comprising the sequence set forth in SEQ ID NO:925. In some embodiments, the antibody comprises: a VH sequence comprising the sequence set forth in SEQ ID NO:645 and a VL sequence comprising the sequence set forth in SEQ ID NO:646. In some embodiments, the antibody comprises: a heavy chain comprising the sequence set forth in SEQ ID NO:926 and a light chain comprising the sequence set forth in SEQ ID NO:927.

In some embodiments, the antibody comprises: a VH-CDR1 comprising the sequence set forth in SEQ ID NO:779; a VH-CDR2 comprising the sequence set forth in SEQ ID NO:780; a VH-CDR3 comprising the sequence set forth in SEQ ID NO:781; a VL-CDR1 comprising the sequence set forth in SEQ ID NO:782; a VL-CDR2 comprising the sequence set forth in SEQ ID NO:783; and a VL-CDR3 comprising the sequence set forth in SEQ ID NO:784.

In some embodiments, the antibody comprises: a VH-CDR1 comprising the sequence set forth in SEQ ID NO:872; a VH-CDR2 comprising the sequence set forth in SEQ ID NO:873; a VH-CDR3 comprising the sequence set forth in SEQ ID NO:874; a VL-CDR1 comprising the sequence set forth in SEQ ID NO:875; a VL-CDR2 comprising the sequence set forth in SEQ ID NO:876; and a VL-CDR3 comprising the sequence set forth in SEQ ID NO:877.

In some embodiments, the antibody comprises: a VH-CDR1 comprising the sequence set forth in SEQ ID NO:884; a VH-CDR2 comprising the sequence set forth in SEQ ID NO:885; a VH-CDR3 comprising the sequence set forth in SEQ ID NO:886; a VL-CDR1 comprising the sequence set forth in SEQ ID NO:887; a VL-CDR2 comprising the sequence set forth in SEQ ID NO:888; and a VL-CDR3 comprising the sequence set forth in SEQ ID NO:889.

In some embodiments, the antibody comprises: a VH sequence comprising the sequence set forth in SEQ ID NO:868 and a VL sequence comprising the sequence set forth in SEQ ID NO:869. In some embodiments, the antibody comprises: a VH sequence comprising the sequence set forth in SEQ ID NO:189 and a VL sequence comprising the sequence set forth in SEQ ID NO:190. In some embodiments, the antibody comprises: a VH sequence comprising the sequence set forth in SEQ ID NO:836 and a VL sequence comprising the sequence set forth in SEQ ID NO:837.

In some embodiments, the antibody comprises: a heavy chain comprising the sequence set forth in SEQ ID NO:920 and a light chain comprising the sequence set forth in SEQ ID NO:921.

In some embodiments, the antibody comprises: a VH-CDR1 comprising the sequence set forth in SEQ ID NO:878; a VH-CDR2 comprising the sequence set forth in SEQ ID NO:879; a VH-CDR3 comprising the sequence set forth in SEQ ID NO:880; a VL-CDR1 comprising the sequence set forth in SEQ ID NO:881; a VL-CDR2 comprising the sequence set forth in SEQ ID NO:882; and a VL-CDR3 comprising the sequence set forth in SEQ ID NO:883.

In some embodiments, the antibody comprises: a VH-CDR1 comprising the sequence set forth in SEQ ID NO:267; a VH-CDR2 comprising the sequence set forth in SEQ ID NO:268; a VH-CDR3 comprising the sequence set forth in SEQ ID NO:269; a VL-CDR1 comprising the sequence set forth in SEQ ID NO:270; a VL-CDR2 comprising the sequence set forth in SEQ ID NO:271; and a VL-CDR3 comprising the sequence set forth in SEQ ID NO:272.

In some embodiments, the antibody comprises: a VH sequence comprising the sequence set forth in SEQ ID NO:870 and a VL sequence comprising the sequence set forth in SEQ ID NO:871. In some embodiments, the antibody comprises: a VH sequence comprising the sequence set forth in SEQ ID NO:303 and a VL sequence comprising the sequence set forth in SEQ ID NO:304. In some embodiments, the antibody comprises: a heavy chain comprising the sequence set forth in SEQ ID NO:922 and a light chain comprising the sequence set forth in SEQ ID NO:923.

In some embodiments, the antibody competes for binding to human TF with the antibody designated 25A, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody competes for binding to human TF with the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody competes for binding to human TF with the antibody designated 25A, the antibody designated 25A5, the antibody designated 25A5-T, the antibody designated 25G, the antibody designated 25G1, or the antibody designated 25G9.

In some embodiments, the antibody binds to the same human TF epitope bound by the antibody designated 25A, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody binds to the same human TF epitope bound by the antibody designated 43B, the antibody designated 43B1, the antibody designated 43B7, the antibody designated 43D, the antibody designated 43D7, the antibody designated 43D8, the antibody designated 43E, or the antibody designated 43Ea. In some embodiments, the antibody binds to the same human TF epitope bound by the antibody designated 25A, the antibody designated 25A5, the antibody designated 25A5-T, the antibody designated 25G, the antibody designated 25G1, or the antibody designated 25G9.

In some embodiments, the antibody does not inhibit human thrombin generation as determined by thrombin generation assay (TGA), does not reduce the thrombin peak on a thrombin generation curve (Peak IIa) compared to an isotype control, does not increase the time from the assay start to the thrombin peak on a thrombin generation curve (ttPeak) compared to an isotype control, does not decrease the endogenous thrombin potential (ETP) as determined by the area under a thrombin generation curve compared to an isotype control, allows human thrombin generation as determined by thrombin generation assay (TGA), maintains the thrombin peak on a thrombin generation curve (Peak IIa) compared to an isotype control, maintains the time from the assay start to the thrombin peak on a thrombin generation curve (ttPeak) compared to an isotype control, preserves the endogenous thrombin potential (ETP) as determined by the area under a thrombin generation curve compared to an isotype control, binds human TF at a human TF binding site that is distinct from a human TF binding site bound by human FX, does not interfere with the ability of TF:FVIIa to convert FX into FXa, and does not compete for binding to human TF with FVIIa.

In some embodiments, the three heavy chain CDRs and the three light chain CDRs are determined using exemplary, Kabat, Chothia, AbM, Contact, or IMGT numbering.

In some embodiments, the antibody specifically binds to cynomolgus TF. In some embodiments, the antibody specifically binds to mouse TF. In some embodiments, the antibody specifically binds to rabbit TF. In some embodiments, the antibody specifically binds to pig TF.

In some embodiments, the disease involves vascular inflammation. In some embodiments, the disease involves local inflammation. In some embodiments, the disease involves systemic inflammation.

In some embodiments, the disease involves infiltration of mononuclear cells and/or granulocytes. In some embodiments, the mononuclear cells comprise macrophages and/or lymphocytes. In some embodiments, the granulocytes comprise neutrophils and/or eosinophils.

In some embodiments, the inflammatory disease is selected from the group consisting of: colitis, inflammatory bowel disease, arthritis, acute lung injury, acute respiratory distress syndrome (ARDS), Respiratory Syncytial Virus (RSV), myocardial infarction, and severe acute respiratory syndrome coronavirus 2 (SARS-CoV-2)

In some embodiments, upon administration to a subject, the antibody reduces the total leukocyte count. In some embodiments, the total leukocyte count is determined by light microscopy.

In some embodiments, upon administration to a subject, the antibody reduces the total number of granulocytes. In some embodiments, the granulocytes comprise neutrophils. In some embodiments, the granulocytes comprise eosinophils. In some embodiments, the total number of granulocytes is determined by immunohistochemical (IHC) analysis or bronco-alveolar lavage (BAL) fluid differential cell count. In some embodiments, the granulocytes are in the alveoli. In some embodiments, the granulocytes are in the interstitial fluid.

In some embodiments, upon administration to a subject, the antibody reduces the total number of mononuclear cells. In some embodiments, the mononuclear cells comprise macrophages. In some embodiments, the macrophages comprise M1 macrophages. In some embodiments, the mononuclear cells comprise lymphocytes. In some embodiments, the mononuclear cells comprise monocytes. In some embodiments, the total number of mononuclear cells is determined by immunohistochemical (IHC) analysis or bronco-alveolar lavage (BAL) fluid differential cell count. In some embodiments, the mononuclear cells are in the alveoli. In some embodiments, the mononuclear cells are in the interstitial fluid.

In some embodiments, upon administration to a subject, the subject maintains or increases body weight relative to baseline levels. In some embodiments, upon administration to a subject, the antibody maintains or increases body weight relative to a different anti-inflammatory therapeutic. In some embodiments, upon administration to a subject, the antibody reduces the spleen size or reverses spleen enlargement relative to baseline levels.

In some embodiments, upon administration to a subject, the antibody reduces the spleen size or reverses splenomegaly relative to a different anti-inflammatory therapeutic. In some embodiments, the spleen size or splenomegaly is determined using palpation, percussion, ultrasound, computerized tomography (CT) scan or magnetic resonance imagining (MRI).

In some embodiments, the inflammatory disease is acute lung injury or ARDS. In some embodiments, upon administration to a subject, the antibody increases net alveolar fluid clearance relative to baseline levels. In some embodiments, upon administration to a subject, the antibody increases net alveolar fluid clearance relative to a different anti-inflammatory therapeutic. In some embodiments, net alveolar fluid clearance is determined by measuring sequential edema fluid protein concentrations. In some embodiments, the sequential edema fluid protein concentrations are measured with ELISA.

In some embodiments, the inflammatory disease is SARS-Cov-2. In some embodiments, upon administration to a subject, the subject maintains or increases body weight relative to baseline levels. In some embodiments, upon administration to a subject, the antibody maintains or increases body weight relative to a different anti-inflammatory therapeutic.

In some embodiments, upon administration to a subject, the antibody reduces the concentration of inflammatory cytokines and chemokines relative to baseline levels. In some embodiments, upon administration to a subject, the antibody reduces the concentration of inflammatory cytokines and chemokines relative to a different anti-inflammatory therapeutic. In some embodiments, the inflammatory cytokines and chemokines are in bronco-alveolar lavage (BAL) samples. In some embodiments, the inflammatory cytokines and chemokines are in lung homogenate samples. In some embodiments, the inflammatory cytokines and chemokines comprise one or more of: IL-1a, IL-10, IL-2, IL-4, IL-5, IL-6, IL-8, IL-10, IFNγ, GM-CSF, TNFα, CCL2, CCL3, CCL4, CCL5, CCL19, CCL20, CCL25, CXCL1, CXCL2, and CXCL10. In some embodiments, the inflammatory cytokines and chemokines are measured using ELISA or Luminex Multiplex Assay. In some embodiments, the inflammatory cytokines and chemokines comprise VEGF. In some embodiments, the inflammatory cytokines and chemokines comprise one or more of: GMCSF, VEGF, IL17F, IL-1 beta, IL-6, IFNγ, IL-8, and KC.

In some embodiments, the inflammatory disease is a viral infection. In some embodiments, upon administration to a subject, the antibody increases anti-inflammatory cytokines and chemokines relative to baseline levels. In some embodiments, upon administration to a subject, the antibody increases anti-inflammatory cytokines and chemokines relative to a different anti-inflammatory therapeutic. In some embodiments, the anti-inflammatory cytokines and chemokines comprise one or more of: IL-10 and IL27p28. In some embodiments, the anti-inflammatory cytokines and chemokines are in bronco-alveolar lavage (BAL) samples. In some embodiments, the inflammatory cytokines and chemokines are measured using multiplex electrochemiluminescence MSD assay. In some embodiments, the inflammatory cytokines and chemokines are measured using Luminex Multiplex Assay.

In some embodiments, the inflammatory disease is RSV. In some embodiments, upon administration to a subject, the antibody reduces fibrosis in the lungs relative to baseline levels. In some embodiments, upon administration to a subject, the antibody reduces fibrosis in the lungs relative to a different anti-inflammatory therapeutic. In some embodiments, the fibrosis is determined by IHC analysis or by Quantitative High Resolution Computed Tomography (qHRCT).

In some embodiments, the inflammatory disease is arthritis. In some embodiments, upon administration to a subject, the antibody reduces the concentration of inflammatory cytokines and chemokines relative to baseline levels. In some embodiments, upon administration to a subject, the antibody reduces the concentration of inflammatory cytokines and chemokines relative to a different anti-inflammatory therapeutic. In some embodiments, the inflammatory cytokines and chemokines comprise one or more of: IL-1a, IL-10, IL-2, IL-4, IL-5, IL-6, IL-8, IL-10, IFNγ, GM-CSF, TNFα, CCL2, CCL3, CCL4, CCL5 CCL19, CCL20, CCL25, CXCL1, CXCL2, and CXCL10.

In some embodiments, the inflammatory disease is colitis or inflammatory bowel disease. In some embodiments, upon administration to a subject, the antibody results in a normal stool consistency or hardens the subject's stool consistency relative to baseline levels. In some embodiments, upon administration to a subject, the antibody results in a normal stool consistency or hardens the subject's stool consistency relative to a different anti-inflammatory therapeutic. In some embodiments, the stool consistency is determined using the Bristol Stool Scale. In some embodiments, upon administration to a subject, the antibody reduces blood or results in the absence of blood in the subject's stool relative to baseline levels. In some embodiments, upon administration to a subject, the antibody reduces blood or results in the absence of blood in the subject's stool relative to a different anti-inflammatory therapeutic. In some embodiments, the blood in the subject's stool is measured using a hemoccult test. In some embodiments, upon administration to a subject, the antibody reduces the concentration of inflammatory cytokines and chemokines relative to baseline levels. In some embodiments, upon administration to a subject, the antibody reduces the concentration of inflammatory cytokines and chemokines relative to a different anti-inflammatory therapeutic. In some embodiments, the inflammatory cytokines and chemokines comprise one or more of: IL-1a, IL-10, IL-2, IL-4, IL-5, IL-6, IL-8, IL-10, IFNγ, GM-CSF, TNFα, CCL2, CCL3, CCL4, CCL5, CCL19, CCL20, CCL25, CXCL1, CXCL2, and CXCL10.

In some embodiments, the inflammatory disease is myocardial infarction.

In some embodiments, upon administration to a subject, the antibody reduces infarct size relative to baseline levels. In some embodiments, upon administration to a subject, the antibody reduces infarct size relative to a different anti-inflammatory therapeutic. In some embodiments, upon administration to a subject, the antibody increases left ventricular ejection fraction relative to baseline levels. In some embodiments, upon administration to a subject, the antibody increases left ventricular ejection fraction relative to a different anti-inflammatory therapeutic. In some embodiments, upon administration to a subject, the antibody decreases left ventricular end diastolic volume relative to baseline levels. In some embodiments, upon administration to a subject, the antibody decreases left ventricular end diastolic volume relative to a different anti-inflammatory therapeutic. In some embodiments, upon administration to a subject, the antibody decreases inflammatory cell recruitment in the infarcted myocardium relative to baseline levels. In some embodiments, upon administration to a subject, the antibody decreases inflammatory cell recruitment in the infarcted myocardium relative to a different anti-inflammatory therapeutic. In some embodiments, the inflammatory cells are selected from CD45+, CD11b⁺, Ly6C^{hi}, CD45⁺/CD90.2⁻/NK1.1⁻/CD11b⁺, CD45⁺/CD90.2⁻/NK1.1⁻/CD11b⁺/Ly6C^{hi}, and CD45⁺/CD90.2⁻/NK1.1⁻/CD11b⁺/Ly6C^{lo}. In some embodiments, the inflammatory cell recruitment is measured using flow cytometry.

In some embodiments, upon administration to a subject, the antibody results in a reduced need for systemic steroids. In some embodiments, the different anti-inflammatory therapeutic comprises one or more of: a non-steroidal anti-inflammatory drug (NSAID), a steroidal anti-inflammatory drug, a beta-agonist, an anticholinergic agent, an antihistamine, and a methyl xanthine. In some embodiments, the different anti-inflammatory therapeutic comprises any one of: an IL-6 inhibitor, anti-GM-CSF, anti-TNFa, anti-IL-1a, dexamethasone, a chemokine and chemokine receptor antagonist, and a JAK inhibitor.

In some embodiments of the present disclosure, an inflammatory disease is treated with an antibody or ADC provided herein that binds human TF at a human TF binding site that is distinct from a human TF binding site bound by human FVIIa. It is also contemplated that an antibody or ADC provided herein that does not bind human TF at a human TF binding site that is distinct from a human TF binding site bound by human FVIIa may be useful for treating the inflammatory diseases. For example, such antibodies may be useful for treatment of inflammatory diseases that are characterized by thrombosis.

In some embodiments, the antibody is administered daily. In some embodiments, the antibody is administered weekly. In some embodiments, the antibody is administered biweekly. In some embodiments, the antibody is administered monthly.

### BRIEF DESCRIPTION OF THE DRAWINGS

These and other features, aspects, and advantages of the present invention will become better understood with regard to the following description, and accompanying drawings, where:

FIG. 1 includes a table showing some common characteristics of acute lung injury (ALI) and acute respiratory distress syndrome (ARDS) in humans.

FIG. 2 includes a schematic showing the qualitative system used for body condition scoring. (See Examples).

FIG. 3 includes a plot showing the percent body weight in mice receiving the indicated treatments in the DSS-colitis model study.

FIG. 4 includes a plot showing the disease activity scores for mice receiving the indicated treatment in the DSS-colitis model study.

FIG. 5 includes a plot showing the body condition scores over the course of the study in mice receiving the indicated treatments in the DSS-colitis model study.

FIG. 6 includes a plot showing the mean weight of mice at the end of the study after having received the indicated treatment in the DSS-colitis model study.

FIG. 7 includes a plot showing the percent weight change in body weight relative to baseline levels in mice receiving the indicated treatments in the ALI model study.

FIG. 8A include plots showing the total leukocyte, total macrophage, and total lymphocyte count in bronchoalveolar lavage (BAL) fluid samples from mice at the end of the study, after having received the indicated treatments in the ALI model study. FIG. 8B include plots showing the total neutrophil and total eosinophil counts in bronchoalveolar lavage (BAL) fluid samples from mice at the end of the study, after having received the indicated treatments in the ALI model study.

FIG. 9 includes a plot showing the results of the histopathological qualitative scoring to compare neutrophil infiltration in the interstitium and alveoli & bronchioles and infiltration of mononuclear cells into the perivascular and peribronchiolar tissue from mice that received the indicated treatments in the ALI model study.

FIG. 10A and FIG. 10B include plots showing the mean inflammatory cytokine and chemokine concentrations (±SEM) measured in BAL fluid from mice having received the indicated treatments in the ALI model study.

FIG. 11 includes a plot showing the mean BAL differential cell count (total leukocytes) measured in mice that received the indicated treatments in the Respiratory Syncytial Virus (RSV) model study.

FIG. 12 includes plots showing the BAL differential measurements for macrophages, neutrophils, and lymphocytes in mice that received the indicated treatments in the Respiratory Syncytial Virus (RSV) model study.

FIG. 13 includes a schematic showing the study schedule for a DSS-induced colitis model.

FIG. 14 includes a plot showing the percent weight changes in DSS mice that received the indicated treatments.

FIG. 15 includes a plot showing the effect of the indicated treatments on the disease activity index (DAI) score in the DSS model.

FIG. 16 includes a plot showing the effect of the indicated treatments on the colon density (i.e. colon weight/colon length) in DSS model.

FIG. 17 includes a plot showing the effect of the indicated treatments on the spleen weight in DSS model.

FIG. 18A includes plots showing the effect of the indicated treatments on levels of inflammatory cytokines in the Poly I:C model model.

FIG. 18B includes plots showing the effect of the indicated treatments on levels of anti-inflammatory cytokines in the Poly I:C model model.

FIG. 19 includes a plot showing the effect of the indicated treatments on body weight in the Poly I:C model model.

FIG. 20 includes echocardiogram images showing the effect of anti-TF antibody and isotype control treatment on infarct size in a myocardial infarction model.

FIG. 21 includes plots showing the effect of anti-TF and isotype control treatment on left ventricular ejection fraction and left ventricular end diastolic volume in a myocardial infarction model.

FIG. 22 and FIG. 23 include plots showing reduced recruitment of inflammatory cells with anti-TF treatment in a myocardial infarction model.

### DETAILED DESCRIPTION

### 1. Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

The terms “Tissue Factor,” “TF,” “platelet tissue factor,” “factor III,” “thromboplastin,” and “CD142” are used interchangeably herein to refer to TF, or any variants (e.g., splice variants and allelic variants), isoforms, and species homologs of TF that are naturally expressed by cells, or that are expressed by cells transfected with a TF gene. In some aspects, the TF protein is a TF protein naturally expressed by a primate (e.g., a monkey or a human), a rodent (e.g., a mouse or a rat), a dog, a camel, a cat, a cow, a goat, a horse, a pig or a sheep. In some aspects, the TF protein is human TF (hTF; SEQ ID NO:809). In some aspects, the TF protein is cynomolgus TF (cTF; SEQ ID NO:813). In some aspects, the TF protein is mouse TF (mTF; SEQ ID NO:817). In some aspects, the TF protein is pig TF (pTF; SEQ ID NO:824). TF is a cell surface receptor for the serine protease factor VIIa. It is often times constitutively expressed by certain cells surrounding blood vessels and in some disease settings.

The term “antibody-drug conjugate” or “ADC” refers to a conjugate comprising an antibody conjugated to one or more cytotoxic agents, optionally through one or more linkers. The term “anti-TF antibody-drug conjugate” or “anti-TF ADC” refers to a conjugate comprising an anti-TF antibody conjugated to one or more cytotoxic agents, optionally through one or more linkers.

The term “cytotoxic agent,” as used herein, refers to a substance that inhibits or prevents a cellular function and/or causes cell death or destruction. The cytotoxic agent can be an anti-angiogenic agent, a pro-apoptotic agent, an anti-mitotic agent, an anti-kinase agent, an alkylating agent, a hormone, a hormone agonist, a hormone antagonist, a chemokine, a drug, a prodrug, a toxin, an enzyme, an antimetabolite, an antibiotic, an alkaloid, or a radioactive isotope. Exemplary cytotoxic agents include calicheamycin, camptothecin, carboplatin, irinotecan, SN-38, carboplatin, camptothecan, cyclophosphamide, cytarabine, dacarbazine, docetaxel, dactinomycin, daunorubicin, doxorubicin, doxorubicin, etoposide, idarubicin, topotecan, vinca alkaloid, maytansinoid, maytansinoid analog, pyrrolobenzodiazepine, taxoid, duocarmycin, dolastatin, auristatin, and derivatives thereof.

A “linker” refers to a molecule that connects one composition to another, e.g., an antibody to an agent. Linkers described herein can conjugate an antibody to a cytotoxic agent. Exemplary linkers include a labile linker, an acid labile linker, a photolabile linker, a charged linker, a disulfide-containing linker, a peptidase-sensitive linker, a β-glucuronide-linker, a dimethyl linker, a thio-ether linker, and a hydrophilic linker. A linker can be cleavable or non-cleavable.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (VH) and a heavy chain constant region (C_{H}). The heavy chain constant region typically comprises three domains, abbreviated C_{H1}, C_{H2}, and C_{H3}. Each light chain typically comprises a light chain variable region (VL) and a light chain constant region. The light chain constant region typically comprises one domain, abbreviated CL.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

The term “alternative scaffold” refers to a molecule in which one or more regions may be diversified to produce one or more antigen-binding domains that specifically bind to an antigen or epitope. In some embodiments, the antigen-binding domain binds the antigen or epitope with specificity and affinity similar to that of an antibody. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody®), ankyrin repeats (e.g., DARPins), gamma-B-crystallin/ubiquitin (e.g., Affilins), CTLD3 (e.g., Tetranectins), Fynomers, and (LDLR-A module) (e.g., Avimers). Additional information on alternative scaffolds is provided in Binz et al., Nat. Biotechnol., 2005 23:1257-1268; Skerra, Current Opin. in Biotech., 2007 18:295-304; and Silacci et al., J. Biol. Chem., 2014, 289:14392-14398; each of which is incorporated by reference in its entirety.

The term “antigen-binding domain” means the portion of an antibody that is capable of specifically binding to an antigen or epitope. One example of an antigen-binding domain is an antigen-binding domain formed by a V_{H}-V_{L} dimer of an antibody. Another example of an antigen-binding domain is an antigen-binding domain formed by diversification of certain loops from the tenth fibronectin type III domain of an Adnectin. Antigen-binding domains can be found in various contexts including antibodies and chimeric antigen receptors (CARs), for example CARs derived from antibodies or antibody fragments such as scFvs.

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “Fc region” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125:S41-52, incorporated by reference in its entirety. The Fc region may be a naturally occurring Fc region, or an Fc region modified as described in the art or elsewhere in this disclosure.

The VH and VL regions may be further subdivided into regions of hypervariability (“hypervariable regions (HVRs);” also called “complementarity determining regions” (CDRs)) interspersed with regions that are more conserved. The more conserved regions are called framework regions (FRs). Each VH and VL generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, VID, incorporated by reference in its entirety.

A “Complementary Determining Region (CDR)” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDRs are variable region sequences interspersed within the framework region sequences. CDRs are well recognized in the art and have been defined by, for example, Kabat as the regions of most hypervariability within the antibody variable (V) domains. See Kabat et al., J Biol Chem, 1977, 252:6609-6616 and Kabat, Adv Protein Chem, 1978, 32:1-75, each of which is incorporated by reference in its entirety. CDRs have also been defined structurally by Chothia as those residues that are not part of the conserved β-sheet framework, and thus are able to adapt different conformations. See Chothia and Lesk, J Mol Biol, 1987, 196:901-917, incorporated by reference in its entirety. Both the Kabat and Chothia nomenclatures are well known in the art. AbM, Contact and IMGT also defined CDRs. CDR positions within a canonical antibody variable domain have been determined by comparison of numerous structures. See Morea et al., Methods, 2000, 20:267-279 and A₁-Lazikani et al., J Mol Biol, 1997, 273:927-48, each of which is incorporated by reference in its entirety. Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra). Such terminology is well known to those skilled in the art.

A number of hypervariable region delineations are in use and are included herein. The Kabat CDRs are based on sequence variability and are the most commonly used. See Kabat et al. (1992) Sequences of Proteins of Immunological Interest, DIANE Publishing: 2719, incorporated by reference in its entirety. Chothia refers instead to the location of the structural loops (Chothia and Lesk, supra). The AbM hypervariable regions represent a compromise between the Kabat CDRs and Chothia structural loops, and are used by Oxford Molecular's AbM antibody modeling software. The Contact hypervariable regions are based on an analysis of the available complex crystal structures. The residues from each of these hypervariable regions are noted in Table 1.

More recently, a universal numbering system ImMunoGeneTics (IMGT) Information System™ has been developed and widely adopted. See Lefranc et al., Dev Comp Immunol, 2003, 27:55-77, incorporated by reference in its entirety. IMGT is an integrated information system specializing in immunoglobulins (IG), T cell receptors (TR) and major histocompatibility complex (MHC) of human and other vertebrates. The IMGT CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. Correspondence between the Kabat, Chothia and IMGT numbering is also well known in the art (Lefranc et al., supra). An Exemplary system, shown herein, combines Kabat and Chothia CDR definitions.

TABLE 1

| | Exemplary | | | | | |
|---|---|---|---|---|---|---|
| | (Kabat + | | | | | |
| | Chothia) | Kabat | Chothia | AbM | Contact | IMGT |
| VH CDR1 | 26-35 | 31-35 | 26-32 | 26-35 | 30-35 | 27-38 |
| VH CDR2 | 50-65 | 50-65 | 52a-55 | 50-58 | 47-58 | 56-65 |
| VH CDR3 | 95-102 | 95-102 | 96-101 | 95-102 | 93-101 | 105-117 |
| VL CDR1 | 24-34 | 24-34 | 26-32 | 24-34 | 30-36 | 27-38 |
| VL CDR2 | 50-56 | 50-56 | 50-52 | 50-56 | 46-55 | 56-65 |
| VL CDR3 | 89-97 | 89-97 | 91-96 | 89-97 | 89-96 | 105-117 |

The light chain from any vertebrate species can be assigned to one of two types, called kappa (x) and lambda (k), based on the sequence of its constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable domain, which contains the antigen-binding site. The constant domain contains the C_{H1}, C_{H2} and C_{H3} domains of the heavy chain and the CL domain of the light chain.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen-binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). Any suitable linker may be used. In some embodiments, the linker is a (GGGGS)ₙ(SEQ ID NO:823). In some embodiments, n=1, 2, 3, 4, 5, or 6. See Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminal of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used.

The term “single domain antibody” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies.

A “multispecific antibody” is an antibody that comprises two or more different antigen-binding domains that collectively specifically bind two or more different epitopes. The two or more different epitopes may be epitopes on the same antigen (e.g., a single TF molecule expressed by a cell) or on different antigens (e.g., a TF molecule and a non-TF molecule). In some aspects, a multi-specific antibody binds two different epitopes (i.e., a “bispecific antibody”). In some aspects, a multi-specific antibody binds three different epitopes (i.e., a “trispecific antibody”). In some aspects, a multi-specific antibody binds four different epitopes (i.e., a “quadspecific antibody”). In some aspects, a multi-specific antibody binds five different epitopes (i.e., a “quintspecific antibody”). In some aspects, a multi-specific antibody binds 6, 7, 8, or more different epitopes. Each binding specificity may be present in any suitable valency. Examples of multispecific antibodies are provided elsewhere in this disclosure.

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” or “isolated nucleic acid” is an antibody or nucleic acid that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. In some embodiments, an isolated antibody may include an antibody in situ within recombinant cells, since at least one component of the antibody's natural environment is not present. In some aspects, an isolated antibody or isolated nucleic acid is prepared by at least one purification step. In some embodiments, an isolated antibody or isolated nucleic acid is purified to at least 80%, 85%, 90%, 95%, or 99% by weight. In some embodiments, an isolated antibody or isolated nucleic acid is purified to at least 80%, 85%, 90%, 95%, or 99% by volume. In some embodiments, an isolated antibody or isolated nucleic acid is provided as a solution comprising at least 85%, 90%, 95%, 98%, 99% to 100% antibody or nucleic acid by weight. In some embodiments, an isolated antibody or isolated nucleic acid is provided as a solution comprising at least 85%, 90%, 95%, 98%, 99% to 100% antibody or nucleic acid by volume.

“Affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some aspects, the affinity of a TF antibody for a non-target molecule is less than about 50% of the affinity for TF. In some aspects, the affinity of a TF antibody for a non-target molecule is less than about 40% of the affinity for TF. In some aspects, the affinity of a TF antibody for a non-target molecule is less than about 30% of the affinity for TF. In some aspects, the affinity of a TF antibody for a non-target molecule is less than about 20% of the affinity for TF. In some aspects, the affinity of a TF antibody for a non-target molecule is less than about 10% of the affinity for TF. In some aspects, the affinity of a TF antibody for a non-target molecule is less than about 1% of the affinity for TF. In some aspects, the affinity of a TF antibody for a non-target molecule is less than about 0.1% of the affinity for TF.

In some embodiments, specifically binding refers to an antibody binding with an affinity of less than 1 nM. In some embodiments, specifically binding refers to an antibody binding with an affinity of less than 10 nM. In some embodiments, specifically binding refers to an antibody binding with an affinity of less than 50 nM. In some embodiments, specifically binding refers to an antibody binding with an affinity of less than 100 nM. In some embodiments, specifically binding refers to an antibody binding with an affinity of less than 200 nM. In some embodiments, specifically binding refers to an antibody binding with an affinity of less than 300 nM. In some embodiments, specifically binding refers to an antibody binding with an affinity of less than 200 nM, 300 nM, 400 nM or 500 nM. In some embodiments, specifically binding refers to an antibody binding with an affinity of less than 0 nM, 10 nM, 20 nM, 30 nM, 40 nM, 50 nM, 60 nM, 70 nM, 80 nM, 90 nM, or 100 nM.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ. In some embodiments, the affinity of an antibody is described in terms of the K_{D} for an interaction between such antibody and its antigen. For clarity, as known in the art, a smaller K_{D} value indicates a higher affinity interaction, while a larger K_{D} value indicates a lower affinity interaction.

The term “KA” (M⁻¹), as used herein, refers to the association equilibrium constant of a particular antibody-antigen interaction. K_{A}=kₐ/k_{d}.

An “affinity matured” antibody is an antibody with one or more alterations (e.g., in one or more CDRs or FRs) relative to a parent antibody (i.e., an antibody from which the altered antibody is derived or designed) that result in an improvement in the affinity of the antibody for its antigen, compared to the parent antibody which does not possess the alteration(s). In some embodiments, an affinity matured antibody has nanomolar or picomolar affinity for the target antigen. Affinity matured antibodies may be produced using a variety of methods known in the art. For example, Marks et al. (Bio Technology, 1992, 10:779-783, incorporated by reference in its entirety) describes affinity maturation by V_{H} and V_{L} domain shuffling. Random mutagenesis of CDR and/or framework residues is described by, for example, Barbas et al., Proc. Nat. Acad. Sci. U.S.A., 1994, 91:3809-3813; Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896; each of which is incorporated by reference in its entirety.

“Fc effector functions” refer to those biological activities mediated by the Fc region of an antibody, which activities may vary depending on the antibody isotype. Examples of antibody effector functions include C1q binding to activate complement dependent cytotoxicity (CDC), Fc receptor binding to activate antibody-dependent cellular cytotoxicity (ADCC), and antibody dependent cellular phagocytosis (ADCP).

When used herein in the context of two or more antibodies, the term “competes with” or “cross-competes with” indicates that the two or more antibodies compete for binding to an antigen (e.g., TF). In one exemplary assay, TF is coated on a surface and contacted with a first TF antibody, after which a second TF antibody is added. In another exemplary assay, first a TF antibody is coated on a surface and contacted with TF, and then a second TF antibody is added. If the presence of the first TF antibody reduces binding of the second TF antibody, in either assay, then the antibodies compete with each other. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 25%, at least 50%, at least 60%, at least 70%, at least 80%, at least 85%, at least 90%, or at least 95%. A skilled artisan can select the concentrations of the antibodies used in the competition assays based on the affinities of the antibodies for TF and the valency of the antibodies. The assays described in this definition are illustrative, and a skilled artisan can utilize any suitable assay to determine if antibodies compete with each other. Suitable assays are described, for example, in Cox et al., “Immunoassay Methods,” in Assay Guidance Manual[Internet], Updated Dec. 24, 2014 (www.ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety. As provided in Example 8, antibodies of group 25 and antibodies of group 43 compete with each other for binding to human TF, while antibodies from groups 1, 29, 39, and 54 do not compete for binding to human TF with antibodies of groups 25 and 43.

As used herein, an antibody that binds specifically to a human antigen is considered to bind the same antigen of mouse origin when a K_{D} value can be measured on a ForteBio Octet with the mouse antigen. An antibody that binds specifically to a human antigen is considered to be “cross-reactive” with the same antigen of mouse origin when the K_{D} value for the mouse antigen is no greater than 20 times the corresponding K_{D} value for the respective human antigen. For example, the antibody M1593 described in U.S. Pat. Nos. 8,722,044, 8,951,525, and 8,999,333, each of which is herein incorporated by reference for all purposes, the humanized 5G9 antibody described in Ngo et al., 2007, Int J Cancer, 120(6):1261-1267, incorporated by reference in its entirety, and chimeric ALT-836 antibody described in Hong et al, 2012, J Nucl Med, 53(11):1748-1754, incorporated by reference in its entirety, do not bind to mouse TF. As provided in Examples 1 and 6, TF antibodies from groups 25 and 43 bind to mouse TF, e.g., the TF antibodies 25G, 25G1, 25G9, and 43D8 are cross-reactive with mouse TF.

As used herein, an antibody that binds specifically to a human antigen is considered to be “cross-reactive” with the same antigen of cynomolgus monkey origin when the K_{D} value for the cynomolgus monkey antigen is no greater than 15 times the corresponding K_{D} value for the respective human antigen. As provided in Example 1, all tested antibodies from groups 1, 25, 29, 39, 43, and 54 are cross-reactive with cynomolgus monkey TF.

The term “epitope” means a portion of an antigen that is specifically bound by an antibody. Epitopes frequently include surface-accessible amino acid residues and/or sugar side chains and may have specific three dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to TF variants with different point-mutations, or to chimeric TF variants.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of an amino acid with a chemically or functionally similar amino acid. Conservative substitution tables providing similar amino acids are well known in the art. By way of example, the groups of amino acids provided in Tables 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable ofdirecting the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “therapeutically effective amount” or “effective amount” refers to an amount of an antibody or pharmaceutical composition provided herein that, when administered to a subject, is effective to treat a disease or disorder. An effective amount is sufficient to effect a desired results or benefit in a subject. An effective amount can be administered in one or more administrations, applications or dosages and is not intended to be limited to a particular formulation or administration route.

As used herein, the terms “baseline levels” and “baseline” refer to the levels for a parameter (e.g. body weight) immediately prior to treatment or at the time of treatment.

As used herein, the term “subject” means a mammalian subject. Exemplary subjects include humans, monkeys, dogs, cats, mice, rats, cows, horses, camels, goats, rabbits, pigs and sheep. In certain embodiments, the subject is a human. In some embodiments the subject has a disease or condition that can be treated with an antibody provided herein. In some aspects, the disease or condition is an inflammatory disease. In some aspects, the disease or condition involves neovascularization or vascular inflammation.

As used herein, the phrase “subject in need thereof” refers to a subject that exhibits and/or is diagnosed with one or more symptoms or signs of inflammatory disease as described herein.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “cytostatic agent” refers to a compound or composition which arrests growth of a cell either in vitro or in vivo. In some embodiments, a cytostatic agent is an agent that reduces the percentage of cells in S phase. In some embodiments, a cytostatic agent reduces the percentage of cells in S phase by at least about 20%, at least about 40%, at least about 60%, or at least about 80%.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

2. TF Antibodies

2.1. TF Binding

Provided herein are isolated antibodies that specifically bind to TF. In some aspects, the TF is hTF (SEQ ID NO:809). In some aspects, the TF is cTF (SEQ ID NO:813). In some aspects, the TF is mTF (SEQ ID NO:817). In some aspects, the TF is rabbit TF (SEQ ID NO:832). In some aspects, the TF is pTF (SEQ ID NO:824). In some embodiments, the antibodies provided herein specifically bind to hTF (SEQ ID NO:809), cTF (SEQ ID NO:813), mTF (SEQ ID NO:817), rabbit TF (SEQ ID NO:832), and pTF (SEQ ID NO:824). In some embodiments, the antibodies provided herein specifically bind to hTF (SEQ ID NO:809), cTF (SEQ ID NO:813), mTF (SEQ ID NO:817), and pTF (SEQ ID NO:824). In some embodiments, the antibodies provided herein specifically bind to hTF (SEQ ID NO:809), cTF (SEQ ID NO:813), and mTF (SEQ ID NO:817). In some embodiments, the antibodies provided herein specifically bind to hTF (SEQ ID NO:809) and cTF (SEQ ID NO:813). In some embodiments, the antibodies provided herein do not bind mTF (SEQ ID NO:817). In some embodiments, the antibodies provided herein do not bind pTF (SEQ ID NO:824). In some embodiments, the antibodies provided herein do not bind rabbit TF (SEQ ID NO:832).

In various embodiments, the antibodies provided herein specifically bind to the extracellular domain of human TF (SEQ ID NO:810).

In some embodiments, the binding between an antibody provided herein and a variant TF extracellular domain comprising a mutation at amino acid residue 149 of the sequence shown in SEQ ID NO:810 is less than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N.

In some embodiments, the binding between an antibody provided herein and a variant TF extracellular domain comprising a mutation at amino acid residue 68 of the sequence shown in SEQ ID NO:810 is greater than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutation at amino acid residue 68 of the sequence shown in SEQ ID NO:810 is K68N.

In some embodiments, the binding between an antibody provided herein and a variant TF extracellular domain comprising mutations at amino acid residues 171 and 197 of the sequence shown in SEQ ID NO:810 is less than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutations at amino acid residues 171 and 197 of the sequence shown in SEQ ID NO:810 are N171H and T197K.

In some embodiments, the binding between an antibody provided herein and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody provided herein and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a variant TF extracellular domain comprising a mutation at amino acid residue 149 of the sequence shown in SEQ ID NO:810 is less than 50% of the binding between the antibody provided herein and the extracellular domain of TF of the sequence shown in SEQ ID NO:810; the binding between an antibody provided herein and a variant TF extracellular domain comprising a mutation at amino acid residue 68 of the sequence shown in SEQ ID NO:810 is greater than 50% of the binding between the antibody provided herein and the extracellular domain of TF of the sequence shown in SEQ ID NO:810; the binding between an antibody provided herein and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810; and the binding between an antibody provided herein and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutation at amino acid residue 68 of the sequence shown in SEQ ID NO:810 is K68N.

In some embodiments, the binding between an antibody provided herein and a variant TF extracellular domain comprising a mutation at amino acid residue 149 of the sequence shown in SEQ ID NO:810 is less than 50% of the binding between the antibody provided herein and the extracellular domain of TF of the sequence shown in SEQ ID NO:810; the binding between an antibody provided herein and a variant TF extracellular domain comprising a mutation at amino acid residue 68 of the sequence shown in SEQ ID NO:810 is greater than 50% of the binding between the antibody provided herein and the extracellular domain of TF of the sequence shown in SEQ ID NO:810; the binding between an antibody provided herein and a variant TF extracellular domain comprising mutations at amino acid residues 171 and 197 of the sequence shown in SEQ ID NO:810 is less than 50% of the binding between the antibody provided herein and the extracellular domain of TF of the sequence shown in SEQ ID NO:810; the binding between an antibody provided herein and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810; the binding between an antibody provided herein and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810; the binding between an antibody provided herein and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810; the binding between an antibody provided herein and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810; the binding between an antibody provided herein and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810; and the binding between an antibody provided herein and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; the mutation at amino acid residue 68 of the sequence shown in SEQ ID NO:810 is K68N; and the mutations at amino acid residues 171 and 197 of the sequence shown in SEQ ID NO:810 are N171H and T197K.

In some embodiments, the antibodies provided herein are inert in inhibiting human thrombin generation as determined by thrombin generation assay (TGA) compared to a reference antibody M1593, wherein the reference antibody M1593 comprises a VH sequence of SEQ ID NO:821 and a VL sequence of SEQ ID NO:822.

In some embodiments, the antibodies provided herein do not inhibit human thrombin generation as determined by thrombin generation assay (TGA). In certain embodiments, the antibodies provided herein allow human thrombin generation as determined by thrombin generation assay (TGA).

In some embodiments, the antibodies provided herein bind human TF at a human TF binding site that is distinct from a human TF binding site bound by human FX. In certain embodiments, the antibodies provided herein do not interfere with the ability of TF:FVIIa to convert FX into FXa.

In some embodiments, the antibodies provided herein bind human TF at a human TF binding site that is distinct from a human TF binding site bound by human FVIIa. In certain embodiments, the antibodies provided herein do not compete for binding to human TF with human FVIIa.

In some embodiments, the antibodies provided herein bind to the extracellular domain of human TF, bind human TF at a human TF binding site that is distinct from a human TF binding site bound by human FVIIa, bind human TF at a human TF binding site that is distinct from a human TF binding site bound by human FX, and allow human thrombin generation as determined by thrombin generation assay (TGA).

In some embodiments, the antibodies provided herein bind to the extracellular domain of human TF, do not inhibit human thrombin generation as determined by thrombin generation assay (TGA), do not interfere with the ability of TF:FVIIa to convert FX into FXa, and do not compete for binding to human TF with human FVIIa.

In some embodiments, the antibodies provided herein bind to the extracellular domain of human TF at a human TF binding site that is distinct from a human TF binding site bound by human FVIIa, do not inhibit human thrombin generation as determined by thrombin generation assay (TGA), allow human thrombin generation as determined by thrombin generation assay (TGA), bind to human TF at a human TF binding site that is distinct from a human TF binding site bound by human FX, do not interfere with the ability of TF:FVIIa to convert FX into FXa, and do not compete for binding to human TF with human FVIIa.

In some embodiments, the antibodies provided herein inhibit FVIIa-dependent TF signaling.

In some embodiments, the antibodies provided herein reduce lesion size in a swine choroidal neovascularization (CNV) model.

In some embodiments, the antibodies provided herein bind to the extracellular domain of human TF at a human TF binding site that is distinct from a human TF binding site bound by human FVIIa, do not inhibit human thrombin generation as determined by thrombin generation assay (TGA), allow human thrombin generation as determined by thrombin generation assay (TGA), bind to human TF at a human TF binding site that is distinct from a human TF binding site bound by human FX, do not interfere with the ability of TF:FVIIa to convert FX into FXa, do not compete for binding to human TF with human FVIIa, and bind to cynomolgus and mouse TF.

In some embodiments, the antibodies provided herein bind to the extracellular domain of human TF at a human TF binding site that is distinct from a human TF binding site bound by human FVIIa, do not inhibit human thrombin generation as determined by thrombin generation assay (TGA), allow human thrombin generation as determined by thrombin generation assay (TGA), bind to human TF at a human TF binding site that is distinct from a human TF binding site bound by human FX, do not interfere with the ability of TF:FVIIa to convert FX into FXa, do not compete for binding to human TF with human FVIIa, bind to cynomolgus, mouse, and pig TF, and reduce lesion size in a swine choroidal neovascularization (CNV) model.

In some embodiments, the antibodies provided herein bind to the extracellular domain of human TF, inhibit FVIIa-dependent TF signaling, and bind to cynomolgus TF.

2.2. Sequences of TF Antibodies

2.2.1. V_{H} Domains

In some embodiments, an antibody provided herein comprises a VH sequence selected from SEQ ID NOs: 37, 75, 113, 151, 189, 227, 265, 303, 341, 379, 417, 455, 493, 531, 569, 607, 645, 683, 721, and 759. In some embodiments, an antibody provided herein comprises a VH sequence of SEQ ID NO:37. In some embodiments, an antibody provided herein comprises a VH sequence of SEQ ID NO:75. In some embodiments, an antibody provided herein comprises a VH sequence of SEQ ID NO:113. In some embodiments, an antibody provided herein comprises a VH sequence of SEQ ID NO:151. In some embodiments, an antibody provided herein comprises a VH sequence of SEQ ID NO:189. In some embodiments, an antibody provided herein comprises a VH sequence of SEQ ID NO:836. In some embodiments, an antibody provided herein comprises a VH sequence of SEQ ID NO:227. In some embodiments, an antibody provided herein comprises a VH sequence of SEQ ID NO:265. In some embodiments, an antibody provided herein comprises a VH sequence of SEQ ID NO:303. In some embodiments, an antibody provided herein comprises a VH sequence of SEQ ID NO:341. In some embodiments, an antibody provided herein comprises a VH sequence of SEQ ID NO:379. In some embodiments, an antibody provided herein comprises a VH sequence of SEQ ID NO:417. In some embodiments, an antibody provided herein comprises a V_{H} sequence of SEQ ID NO:455. In some embodiments, an antibody provided herein comprises a V_{H} sequence of SEQ ID NO:493. In some embodiments, an antibody provided herein comprises a V_{H} sequence of SEQ ID NO:531. In some embodiments, an antibody provided herein comprises a V_{H} sequence of SEQ ID NO:569. In some embodiments, an antibody provided herein comprises a V_{H} sequence of SEQ ID NO:607. In some embodiments, an antibody provided herein comprises a V_{H} sequence of SEQ ID NO:645. In some embodiments, an antibody provided herein comprises a V_{H} sequence of SEQ ID NO:683. In some embodiments, an antibody provided herein comprises a V_{H} sequence of SEQ ID NO:721. In some embodiments, an antibody provided herein comprises a V_{H} sequence of SEQ ID NO:759.

In some embodiments, an antibody provided herein comprises a V_{H} sequence having at least about 50%, 60%, 70%, 80%, 90%, 95%, or 99% identity to an illustrative V_{H} sequence provided in SEQ ID NOs: 37, 75, 113, 151, 189, 227, 265, 303, 341, 379, 417, 455, 493, 531, 569, 607, 645, 683, 721, and 759. In some embodiments, an antibody provided herein comprises a VH sequence provided in SEQ ID NOs: 37, 75, 113, 151, 189, 227, 265, 303, 341, 379, 417, 455, 493, 531, 569, 607, 645, 683, 721, and 759, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

2.2.2. V_{L} Domains

In some embodiments, an antibody provided herein comprises a V_{L} sequence selected from SEQ ID NOs: 38, 76, 114, 152, 190, 228, 266, 304, 342, 380, 418, 456, 494, 532, 570, 608, 646, 684, 722, and 760. In some embodiments, an antibody provided herein comprises a V_{L} sequence of SEQ ID NO:38. In some embodiments, an antibody provided herein comprises a V_{L} sequence of SEQ ID NO:76. In some embodiments, an antibody provided herein comprises a V_{L} sequence of SEQ ID NO:114. In some embodiments, an antibody provided herein comprises a V_{L} sequence of SEQ ID NO:152. In some embodiments, an antibody provided herein comprises a V_{L} sequence of SEQ ID NO:190. In some embodiments, an antibody provided herein comprises a V_{L} sequence of SEQ ID NO:837. In some embodiments, an antibody provided herein comprises a V_{L} sequence of SEQ ID NO:228. In some embodiments, an antibody provided herein comprises a V_{L} sequence of SEQ ID NO:266. In some embodiments, an antibody provided herein comprises a V_{L} sequence of SEQ ID NO:304. In some embodiments, an antibody provided herein comprises a V_{L} sequence of SEQ ID NO:342. In some embodiments, an antibody provided herein comprises a V_{L} sequence of SEQ ID NO:380. In some embodiments, an antibody provided herein comprises a V_{L} sequence of SEQ ID NO:418. In some embodiments, an antibody provided herein comprises a V_{L} sequence of SEQ ID NO:456. In some embodiments, an antibody provided herein comprises a V_{L} sequence of SEQ ID NO:494. In some embodiments, an antibody provided herein comprises a V_{L} sequence of SEQ ID NO:532. In some embodiments, an antibody provided herein comprises a V_{L} sequence of SEQ ID NO:570. In some embodiments, an antibody provided herein comprises a V_{L} sequence of SEQ ID NO:608. In some embodiments, an antibody provided herein comprises a V_{L} sequence of SEQ ID NO:646. In some embodiments, an antibody provided herein comprises a V_{L} sequence of SEQ ID NO:684. In some embodiments, an antibody provided herein comprises a V_{L} sequence of SEQ ID NO:722. In some embodiments, an antibody provided herein comprises a V_{L} sequence of SEQ ID NO:760.

In some embodiments, an antibody provided herein comprises a V_{L} sequence having at least about 50%, 60%, 70%, 80%, 90%, 95%, or 99% identity to an illustrative V_{L} sequence provided in SEQ ID NOs: 38, 76, 114, 152, 190, 228, 266, 304, 342, 380, 418, 456, 494, 532, 570, 608, 646, 684, 722, and 760. In some embodiments, an antibody provided herein comprises a V_{L} sequence provided in SEQ ID NOs: 38, 76, 114, 152, 190, 228, 266, 304, 342, 380, 418, 456, 494, 532, 570, 608, 646, 684, 722, and 760, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

2.2.3. V_{H}-V_{L} Combinations

In some embodiments, an antibody provided herein comprises a VH sequence selected from SEQ ID NOs: 37, 75, 113, 151, 189, 227, 265, 303, 341, 379, 417, 455, 493, 531, 569, 607, 645, 683, 721, and 759 and a V_{L} sequence selected from SEQ ID NOs: 38, 76, 114, 152, 190, 228, 266, 304, 342, 380, 418, 456, 494, 532, 570, 608, 646, 684, 722, and 760.

In some embodiments, an antibody provided herein comprises a V_{H} sequence of SEQ ID NO:37 and a V_{L} sequence of SEQ ID NO:38. In some embodiments, an antibody provided herein comprises a V_{H} sequence of SEQ ID NO:75 and a V_{L} sequence of SEQ ID NO:76. In some embodiments, an antibody provided herein comprises a V_{H} sequence of SEQ ID NO:113 and a V_{L} sequence of SEQ ID NO:114. In some embodiments, an antibody provided herein comprises a V_{H} sequence of SEQ ID NO: 151 and a V_{L} sequence of SEQ ID NO:152. In some embodiments, an antibody provided herein comprises a V_{H} sequence of SEQ ID NO:189 and a V_{L} sequence of SEQ ID NO:190. In some embodiments, an antibody provided herein comprises a V_{H} sequence of SEQ ID NO:836 and a V_{L} sequence of SEQ ID NO:837. In some embodiments, an antibody provided herein comprises a V_{H} sequence of SEQ ID NO:227 and a V_{L} sequence of SEQ ID NO:228. In some embodiments, an antibody provided herein comprises a V_{H} sequence of SEQ ID NO:265 and a V_{L} sequence of SEQ ID NO:266. In some embodiments, an antibody provided herein comprises a V_{H} sequence of SEQ ID NO:303 and a V_{L} sequence of SEQ ID NO:304. In some embodiments, an antibody provided herein comprises a V_{H} sequence of SEQ ID NO:341 and a V_{L} sequence of SEQ ID NO:342. In some embodiments, an antibody provided herein comprises a V_{H} sequence of SEQ ID NO:379 and a V_{L} sequence of SEQ ID NO:380. In some embodiments, an antibody provided herein comprises a V_{H} sequence of SEQ ID NO:417 and a V_{L} sequence of SEQ ID NO:418. In some embodiments, an antibody provided herein comprises a V_{H} sequence of SEQ ID NO:455 and a V_{L} sequence of SEQ ID NO:456. In some embodiments, an antibody provided herein comprises a V_{H} sequence of SEQ ID NO:493 and a V_{L} sequence of SEQ ID NO:494. In some embodiments, an antibody provided herein comprises a V_{H} sequence of SEQ ID NO:531 and a V_{L} sequence of SEQ ID NO:532. In some embodiments, an antibody provided herein comprises a V_{H} sequence of SEQ ID NO:569 and a V_{L} sequence of SEQ ID NO:570. In some embodiments, an antibody provided herein comprises a V_{H} sequence of SEQ ID NO:607 and a V_{L} sequence of SEQ ID NO:608. In some embodiments, an antibody provided herein comprises a V_{H} sequence of SEQ ID NO:645 and a V_{L} sequence of SEQ ID NO:646. In some embodiments, an antibody provided herein comprises a V_{H} sequence of SEQ ID NO:683 and a V_{L} sequence of SEQ ID NO:684. In some embodiments, an antibody provided herein comprises a V_{H} sequence of SEQ ID NO:721 and a V_{L} sequence of SEQ ID NO:722. In some embodiments, an antibody provided herein comprises a V_{H} sequence of SEQ ID NO:759 and a V_{L} sequence of SEQ ID NO:760.

In some embodiments, an antibody provided herein comprises a V_{H} sequence having at least about 50%, 60%, 70%, 80%, 90%, 95%, or 99% identity to an illustrative V_{H} sequence provided in SEQ ID NOs: 37, 75, 113, 151, 189, 227, 265, 303, 341, 379, 417, 455, 493, 531, 569, 607, 645, 683, 721, and 759, and a V_{L} sequence having at least about 50%, 60%, 70%, 80%, 90%, 95%, or 99% identity to an illustrative V_{L} sequence provided in SEQ ID NOs: 38, 76, 114, 152, 190, 228, 266, 304, 342, 380, 418, 456, 494, 532, 570, 608, 646, 684, 722, and 760. In some embodiments, an antibody provided herein comprises a V_{H} sequence provided in SEQ ID NOs: 37, 75, 113, 151, 189, 227, 265, 303, 341, 379, 417, 455, 493, 531, 569, 607, 645, 683, 721, and 759, with up to 1, 2, 3, 4, 5, 6, 7, 8, 9, 10, 11, 12, 13, 14, 15, 16, 17, 18, 19, 20, 21, 22, 23, 24, or 25 amino acid substitutions, and a V_{L} sequence provided in SEQ ID NOs: 38, 76, 114, 152, 190, 228, 266, 304, 342, 380, 418, 456, 494, 532, 570, 608, 646, 684, 722, and 760, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

2.2.4. CDRs

In some embodiments, an antibody provided herein comprises one to three CDRs of a V_{H} domain selected from SEQ ID NOs: 37, 75, 113, 151, 189, 227, 265, 303, 341, 379, 417, 455, 493, 531, 569, 607, 645, 683, 721, and 759. In some embodiments, an antibody provided herein comprises two to three CDRs of a V_{H} domain selected from SEQ ID NOs: 37, 75, 113, 151, 189, 227, 265, 303, 341, 379, 417, 455, 493, 531, 569, 607, 645, 683, 721, and 759. In some embodiments, an antibody provided herein comprises three CDRs of a V_{H} domain selected from SEQ ID NOs: 37, 75, 113, 151, 189, 227, 265, 303, 341, 379, 417, 455, 493, 531, 569, 607, 645, 683, 721, and 759. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-H1, CDR-H2, or CDR-H3 of SEQ ID NOs: 37, 75, 113, 151, 189, 227, 265, 303, 341, 379, 417, 455, 493, 531, 569, 607, 645, 683, 721, and 759.

In some embodiments, the CDR-H1 is a CDR-H1 of a V_{H} domain selected from SEQ ID NOs: 37, 75, 113, 151, 189, 227, 265, 303, 341, 379, 417, 455, 493, 531, 569, 607, 645, 683, 721, and 759, with up to 1, 2, 3, 4, or 5 amino acid substitutions. In some embodiments, the CDR-H2 is a CDR-H2 of a V_{H} domain selected from SEQ ID NOs: 37, 75, 113, 151, 189, 227, 265, 303, 341, 379, 417, 455, 493, 531, 569, 607, 645, 683, 721, and 759, with up to 1, 2, 3, 4, 5, 6, 7, or 8 amino acid substitutions. In some embodiments, the CDR-H3 is a CDR-H3 of a V_{H} domain selected from SEQ ID NOs: 37, 75, 113, 151, 189, 227, 265, 303, 341, 379, 417, 455, 493, 531, 569, 607, 645, 683, 721, and 759, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a V_{L} domain selected from SEQ ID NOs: 38, 76, 114, 152, 190, 228, 266, 304, 342, 380, 418, 456, 494, 532, 570, 608, 646, 684, 722, and 760. In some embodiments, an antibody provided herein comprises two to three CDRs of a V_{L} domain selected from SEQ ID NOs: 38, 76, 114, 152, 190, 228, 266, 304, 342, 380, 418, 456, 494, 532, 570, 608, 646, 684, 722, and 760. In some embodiments, an antibody provided herein comprises three CDRs of a V_{L} domain selected from SEQ ID NOs: 38, 76, 114, 152, 190, 228, 266, 304, 342, 380, 418, 456, 494, 532, 570, 608, 646, 684, 722, and 760. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-L1, CDR-L2, or CDR-L3 of SEQ ID NOs: 38, 76, 114, 152, 190, 228, 266, 304, 342, 380, 418, 456, 494, 532, 570, 608, 646, 684, 722, and 760.

In some embodiments, the CDR-L1 is a CDR-L1 of a V_{L} domain selected from SEQ ID NOs: 38, 76, 114, 152, 190, 228, 266, 304, 342, 380, 418, 456, 494, 532, 570, 608, 646, 684, 722, and 760, with up to 1, 2, 3, 4, or 5 amino acid substitutions. In some embodiments, the CDR-L2 is a CDR-L2 of a V_{L} domain selected from SEQ ID NOs: 38, 76, 114, 152, 190, 228, 266, 304, 342, 380, 418, 456, 494, 532, 570, 608, 646, 684, 722, and 760, with up to 1, 2, 3, 4, 5, 6, 7, or 8 amino acid substitutions. In some embodiments, the CDR-L3 is a CDR-L3 of a V_{L} domain selected from SEQ ID NOs: 38, 76, 114, 152, 190, 228, 266, 304, 342, 380, 418, 456, 494, 532, 570, 608, 646, 684, 722, and 760, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a V_{H} domain selected from SEQ ID NOs: 37, 75, 113, 151, 189, 227, 265, 303, 341, 379, 417, 455, 493, 531, 569, 607, 645, 683, 721, and 759 and one to three CDRs of a V_{L} domain selected from SEQ ID NOs: 38, 76, 114, 152, 190, 228, 266, 304, 342, 380, 418, 456, 494, 532, 570, 608, 646, 684, 722, and 760. In some embodiments, an antibody provided herein comprises two to three CDRs of a V_{H} domain selected from SEQ ID NOs: 37, 75, 113, 151, 189, 227, 265, 303, 341, 379, 417, 455, 493, 531, 569, 607, 645, 683, 721, and 759 and two to three CDRs of a V_{L} domain selected from SEQ ID NOs: 38, 76, 114, 152, 190, 228, 266, 304, 342, 380, 418, 456, 494, 532, 570, 608, 646, 684, 722, and 760. In some embodiments, an antibody provided herein comprises three CDRs of a V_{H} domain selected from SEQ ID NOs: 37, 75, 113, 151, 189, 227, 265, 303, 341, 379, 417, 455, 493, 531, 569, 607, 645, 683, 721, and 759 and three CDRs of a V_{L} domain selected from SEQ ID NOs: 38, 76, 114, 152, 190, 228, 266, 304, 342, 380, 418, 456, 494, 532, 570, 608, 646, 684, 722, and 760. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-H1, CDR-H2, or CDR-H3 of SEQ ID NOs: 37, 75, 113, 151, 189, 227, 265, 303, 341, 379, 417, 455, 493, 531, 569, 607, 645, 683, 721, and 759 and at least about 50%, 75%, 80%, 85%, 90%, or 95% identity with a CDR-L1, CDR-L2, or CDR-L3 of SEQ ID NOs: 38, 76, 114, 152, 190, 228, 266, 304, 342, 380, 418, 456, 494, 532, 570, 608, 646, 684, 722, and 760. In some embodiments, the CDR-H1 is a CDR-H1 of a V_{H} domain selected from SEQ ID NOs: 37, 75, 113, 151, 189, 227, 265, 303, 341, 379, 417, 455, 493, 531, 569, 607, 645, 683, 721, and 759, with up to 1, 2, 3, 4, or 5 amino acid substitutions; the CDR-H2 is a CDR-H2 of a V_{H} domain selected from SEQ ID NOs: 37, 75, 113, 151, 189, 227, 265, 303, 341, 379, 417, 455, 493, 531, 569, 607, 645, 683, 721, and 759, with up to 1, 2, 3, 4, 5, 6, 7, or 8 amino acid substitutions; the CDR-H3 is a CDR-H3 of a V_{H} domain selected from SEQ ID NOs: 37, 75, 113, 151, 189, 227, 265, 303, 341, 379, 417, 455, 493, 531, 569, 607, 645, 683, 721, and 759, with up to 1, 2, 3, 4, 5, 6, 7, or 8 amino acid substitutions; the CDR-L1 is a CDR-L1 of a V_{L} domain selected from SEQ ID NOs: 38, 76, 114, 152, 190, 228, 266, 304, 342, 380, 418, 456, 494, 532, 570, 608, 646, 684, 722, and 760, with up to 1, 2, 3, 4, 5, or 6 amino acid substitutions; the CDR-L2 is a CDR-L2 of a V_{L} domain selected from SEQ ID NOs: 38, 76, 114, 152, 190, 228, 266, 304, 342, 380, 418, 456, 494, 532, 570, 608, 646, 684, 722, and 760, with up to 1, 2, 3, or 4 amino acid substitutions; and the CDR-L3 is a CDR-L3 of a V_{L} domain selected from SEQ ID NOs: 38, 76, 114, 152, 190, 228, 266, 304, 342, 380, 418, 456, 494, 532, 570, 608, 646, 684, 722, and 760, with up to 1, 2, 3, 4, or 5 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein.

In some embodiments, such variants are not derived from a sequence provided herein and may, for example, be isolated de novo according to the methods provided herein for obtaining antibodies.

In some embodiments, an antibody provided herein comprises a CDR-H3 selected from SEQ ID NOs: 3, 41, 79, 117, 155, 193, 231, 269, 307, 345, 383, 421, 459, 497, 535, 573, 611, 649, 687, and 725, as determined by the Exemplary numbering system. In some aspects, the CDR-H3 has at least about 50%, 75%, 80%, 85%, 90%, or 95% identity with a CDR-H3 of SEQ ID NOs: 3, 41, 79, 117, 155, 193, 231, 269, 307, 345, 383, 421, 459, 497, 535, 573, 611, 649, 687, and 725. In some embodiments, the CDR-H3 is a CDR-H3 selected from SEQ ID NOs: 3, 41, 79, 117, 155, 193, 231, 269, 307, 345, 383, 421, 459, 497, 535, 573, 611, 649, 687, and 725, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H2 selected from SEQ ID NOs: 2, 40, 78, 116, 154, 192, 230, 268, 306, 344, 382, 420, 458, 496, 534, 572, 610, 648, 686, and 724, as determined by the Exemplary numbering system. In some aspects, the CDR-H2 has at least about 50%, 75%, 80%, 85%, 90%, or 95% identity with a CDR-H2 of SEQ ID NOs: 2, 40, 78, 116, 154, 192, 230, 268, 306, 344, 382, 420, 458, 496, 534, 572, 610, 648, 686, and 724. In some embodiments, the CDR-H2 is a CDR-H2 selected from SEQ ID NOs: 2, 40, 78, 116, 154, 192, 230, 268, 306, 344, 382, 420, 458, 496, 534, 572, 610, 648, 686, and 724, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H1 selected from SEQ ID NOs: 1, 39, 77, 115, 153, 191, 229, 267, 305, 343, 381, 419, 457, 495, 533, 571, 609, 647, 685, and 723, as determined by the Exemplary numbering system. In some aspects, the CDR-H1 has at least about 50%, 75%, 80%, 85%, 90%, or 95% identity with a CDR-H1 of SEQ ID NOs: 1, 39, 77, 115, 153, 191, 229, 267, 305, 343, 381, 419, 457, 495, 533, 571, 609, 647, 685, and 723. In some embodiments, the CDR-H1 is a CDR-H1 selected from SEQ ID NOs: 1, 39, 77, 115, 153, 191, 229, 267, 305, 343, 381, 419, 457, 495, 533, 571, 609, 647, 685, and 723, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected from SEQ ID NOs: 3, 41, 79, 117, 155, 193, 231, 269, 307, 345, 383, 421, 459, 497, 535, 573, 611, 649, 687, and 725 and a CDR-H2 selected from SEQ ID NOs: 2, 40, 78, 116, 154, 192, 230, 268, 306, 344, 382, 420, 458, 496, 534, 572, 610, 648, 686, and 724. In some embodiments, an antibody provided herein comprises a CDR-H3 selected from SEQ ID NOs: 3, 41, 79, 117, 155, 193, 231, 269, 307, 345, 383, 421, 459, 497, 535, 573, 611, 649, 687, and 725, a CDR-H2 selected from SEQ ID NOs: 2, 40, 78, 116, 154, 192, 230, 268, 306, 344, 382, 420, 458, 496, 534, 572, 610, 648, 686, and 724, and a CDR-H1 selected from SEQ ID NOs: 1, 39, 77, 115, 153, 191, 229, 267, 305, 343, 381, 419, 457, 495, 533, 571, 609, 647, 685, and 723. In some embodiments, the CDR-H3 has at least about 50%, 75%, 80%, 85%, 90%, or 95% identity with a CDR-H3 of SEQ ID NOs: 3, 41, 79, 117, 155, 193, 231, 269, 307, 345, 383, 421, 459, 497, 535, 573, 611, 649, 687, and 725, the CDR-H2 has at least about 50%, 75%, 80%, 85%, 90%, or 95% identity with a CDR-H2 of SEQ ID NOs: 2, 40, 78, 116, 154, 192, 230, 268, 306, 344, 382, 420, 458, 496, 534, 572, 610, 648, 686, and 724, and the CDR-H1 has at least about 50%, 75%, 80%, 85%, 90%, or 95% identity with a CDR-H1 of SEQ ID NOs: 1, 39, 77, 115, 153, 191, 229, 267, 305, 343, 381, 419, 457, 495, 533, 571, 609, 647, 685, and 723. In some embodiments, the CDR-H3 is a CDR-H3 selected from SEQ ID NOs: 3, 41, 79, 117, 155, 193, 231, 269, 307, 345, 383, 421, 459, 497, 535, 573, 611, 649, 687, and 725, with up to 1, 2, 3, 4, 5, 6, 7, or 8 amino acid substitutions; the CDR-H2 is a CDR-H2 selected from SEQ ID NOs: 2, 40, 78, 116, 154, 192, 230, 268, 306, 344, 382, 420, 458, 496, 534, 572, 610, 648, 686, and 724, with up to 1, 2, 3, 4, 5, 6, 7, or 8 amino acid substitutions; and the CDR-H1 is a CDR-H1 selected from SEQ ID NOs: 1, 39, 77, 115, 153, 191, 229, 267, 305, 343, 381, 419, 457, 495, 533, 571, 609, 647, 685, and 723, with up to 1, 2, 3, 4, or 5 amino acid substitutions. In some aspects, the amino acid substitutions are conservative amino acid substitutions. In some embodiments, the antibody described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L3 selected from SEQ ID NOs: 6, 44, 82, 120, 158, 196, 234, 272, 310, 348, 386, 424, 462, 500, 538, 576, 614, 652, 690, and 728, as determined by the Exemplary numbering system. In some aspects, the CDR-L3 has at least about 50%, 75%, 80%, 85%, 90%, or 95% identity with a CDR-L3 of SEQ ID NOs: 6, 44, 82, 120, 158, 196, 234, 272, 310, 348, 386, 424, 462, 500, 538, 576, 614, 652, 690, and 728. In some embodiments, the CDR-L3 is a CDR-L3 selected from SEQ ID NOs: 6, 44, 82, 120, 158, 196, 234, 272, 310, 348, 386, 424, 462, 500, 538, 576, 614, 652, 690, and 728, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L2 selected from SEQ ID NOs: 5, 43, 81, 119, 157, 195, 233, 271, 309, 347, 385, 423, 461, 499, 537, 575, 613, 651, 689, and 727, as determined by the Exemplary numbering system. In some aspects, the CDR-L2 has at least about 50%, 75%, 80%, 85%, 90%, or 95% identity with a CDR-L2 of SEQ ID NOs: 5, 43, 81, 119, 157, 195, 233, 271, 309, 347, 385, 423, 461, 499, 537, 575, 613, 651, 689, and 727. In some embodiments, the CDR-L2 is a CDR-L2 selected from SEQ ID NOs: 5, 43, 81, 119, 157, 195, 233, 271, 309, 347, 385, 423, 461, 499, 537, 575, 613, 651, 689, and 727, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L1 selected from SEQ ID NOs: 4, 42, 80, 118, 156, 194, 232, 270, 308, 346, 384, 422, 460, 498, 536, 574, 612, 650, 688, and 726, as determined by the Exemplary numbering system. In some aspects, the CDR-L1 has at least about 50%, 75%, 80%, 85%, 90%, or 95% identity with a CDR-L1 of SEQ ID NOs: 4, 42, 80, 118, 156, 194, 232, 270, 308, 346, 384, 422, 460, 498, 536, 574, 612, 650, 688, and 726. In some embodiments, the CDR-L1 is a CDR-L1 selected from SEQ ID NOs: 4, 42, 80, 118, 156, 194, 232, 270, 308, 346, 384, 422, 460, 498, 536, 574, 612, 650, 688, and 726, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L3 selected from SEQ ID NOs: 6, 44, 82, 120, 158, 196, 234, 272, 310, 348, 386, 424, 462, 500, 538, 576, 614, 652, 690, and 728 and a CDR-L2 selected from SEQ ID NOs: 5, 43, 81, 119, 157, 195, 233, 271, 309, 347, 385, 423, 461, 499, 537, 575, 613, 651, 689, and 727. In some embodiments, an antibody provided herein comprises a CDR-L3 selected from SEQ ID NOs: 6, 44, 82, 120, 158, 196, 234, 272, 310, 348, 386, 424, 462, 500, 538, 576, 614, 652, 690, and 728, a CDR-L2 selected from SEQ ID NOs: 5, 43, 81, 119, 157, 195, 233, 271, 309, 347, 385, 423, 461, 499, 537, 575, 613, 651, 689, and 727, and a CDR-L1 selected from SEQ ID NOs: 4, 42, 80, 118, 156, 194, 232, 270, 308, 346, 384, 422, 460, 498, 536, 574, 612, 650, 688, and 726. In some embodiments, the CDR-L3 has at least about 50%, 75%, 80%, 85%, 90%, or 95% identity with a CDR-L3 of SEQ ID NOs: 6, 44, 82, 120, 158, 196, 234, 272, 310, 348, 386, 424, 462, 500, 538, 576, 614, 652, 690, and 728, the CDR-L2 has at least about 50%, 75%, 80%, 85%, 90%, or 95% identity with a CDR-L2 of SEQ ID NOs: 5, 43, 81, 119, 157, 195, 233, 271, 309, 347, 385, 423, 461, 499, 537, 575, 613, 651, 689, and 727, and the CDR-L1 has at least about 50%, 75%, 80%, 85%, 90%, or 95% identity with a CDR-L1 of SEQ ID NOs: 4, 42, 80, 118, 156, 194, 232, 270, 308, 346, 384, 422, 460, 498, 536, 574, 612, 650, 688, and 726. In some embodiments, the CDR-L3 is a CDR-L3 selected from SEQ ID NOs: 6, 44, 82, 120, 158, 196, 234, 272, 310, 348, 386, 424, 462, 500, 538, 576, 614, 652, 690, and 728, with up to 1, 2, 3, 4, or 5 amino acid substitutions; the CDR-L2 is a CDR-L2 selected from SEQ ID NOs: 5, 43, 81, 119, 157, 195, 233, 271, 309, 347, 385, 423, 461, 499, 537, 575, 613, 651, 689, and 727, with up to 1, 2, 3, or 4 amino acid substitutions; and the CDR-L1 is a CDR-L1 selected from SEQ ID NOs: 4, 42, 80, 118, 156, 194, 232, 270, 308, 346, 384, 422, 460, 498, 536, 574, 612, 650, 688, and 726, with up to 1, 2, 3, 4, 5, or 6 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected from SEQ ID NOs: 3, 41, 79, 117, 155, 193, 231, 269, 307, 345, 383, 421, 459, 497, 535, 573, 611, 649, 687, and 725, a CDR-H2 selected from SEQ ID NOs: 2, 40, 78, 116, 154, 192, 230, 268, 306, 344, 382, 420, 458, 496, 534, 572, 610, 648, 686, and 724, a CDR-H1 selected from SEQ ID NOs: 1, 39, 77, 115, 153, 191, 229, 267, 305, 343, 381, 419, 457, 495, 533, 571, 609, 647, 685, and 723, a CDR-L3 selected from SEQ ID NOs: 6, 44, 82, 120, 158, 196, 234, 272, 310, 348, 386, 424, 462, 500, 538, 576, 614, 652, 690, and 728, a CDR-L2 selected from SEQ ID NOs: 5, 43, 81, 119, 157, 195, 233, 271, 309, 347, 385, 423, 461, 499, 537, 575, 613, 651, 689, and 727, and a CDR-L1 selected from SEQ ID NOs: 4, 42, 80, 118, 156, 194, 232, 270, 308, 346, 384, 422, 460, 498, 536, 574, 612, 650, 688, and 726. In some embodiments, the CDR-H3 has at least about 50%, 75%, 80%, 85%, 90%, or 95% identity with a CDR-H3 of SEQ ID NOs: 3, 41, 79, 117, 155, 193, 231, 269, 307, 345, 383, 421, 459, 497, 535, 573, 611, 649, 687, and 725, the CDR-H2 has at least about 50%, 75%, 80%, 85%, 90%, or 95% identity with a CDR-H2 of SEQ ID NOs: 2, 40, 78, 116, 154, 192, 230, 268, 306, 344, 382, 420, 458, 496, 534, 572, 610, 648, 686, and 724, the CDR-H1 has at least about 50%, 75%, 80%, 85%, 90%, or 95% identity with a CDR-H1 of SEQ ID NOs: 1, 39, 77, 115, 153, 191, 229, 267, 305, 343, 381, 419, 457, 495, 533, 571, 609, 647, 685, and 723, the CDR-L3 has at least about 50%, 75%, 80%, 85%, 90%, or 95% identity with a CDR-L3 of SEQ ID NOs: 6, 44, 82, 120, 158, 196, 234, 272, 310, 348, 386, 424, 462, 500, 538, 576, 614, 652, 690, and 728, the CDR-L2 has at least about 50%, 75%, 80%, 85%, 90%, or 95% identity with a CDR-L2 of SEQ ID NOs: 5, 43, 81, 119, 157, 195, 233, 271, 309, 347, 385, 423, 461, 499, 537, 575, 613, 651, 689, and 727, and the CDR-L1 has at least about 50%, 75%, 80%, 85%, 90%, or 95% identity with a CDR-L1 of SEQ ID NOs: 4, 42, 80, 118, 156, 194, 232, 270, 308, 346, 384, 422, 460, 498, 536, 574, 612, 650, 688, and 726. In some embodiments, the CDR-H3 is a CDR-H3 selected from SEQ ID NOs: 3, 41, 79, 117, 155, 193, 231, 269, 307, 345, 383, 421, 459, 497, 535, 573, 611, 649, 687, and 725, with up to 1, 2, 3, 4, 5, 6, 7, or 8 amino acid substitutions; the CDR-H2 is a CDR-H2 selected from SEQ ID NOs: 2, 40, 78, 116, 154, 192, 230, 268, 306, 344, 382, 420, 458, 496, 534, 572, 610, 648, 686, and 724, with up to 1, 2, 3, 4, 5, 6, 7, or 8 amino acid substitutions; the CDR-H1 is a CDR-H1 selected from SEQ ID NOs: 1, 39, 77, 115, 153, 191, 229, 267, 305, 343, 381, 419, 457, 495, 533, 571, 609, 647, 685, and 723, with up to 1, 2, 3, 4, or 5 amino acid substitutions; the CDR-L3 is a CDR-L3 selected from SEQ ID NOs: 6, 44, 82, 120, 158, 196, 234, 272, 310, 348, 386, 424, 462, 500, 538, 576, 614, 652, 690, and 728, with up to 1, 2, 3, 4, or 5 amino acid substitutions; the CDR-L2 is a CDR-L2 selected from SEQ ID NOs: 5, 43, 81, 119, 157, 195, 233, 271, 309, 347, 385, 423, 461, 499, 537, 575, 613, 651, 689, and 727, with up to 1, 2, 3, or 4 amino acid substitutions; and the CDR-L1 is a CDR-L1 selected from SEQ ID NOs: 4, 42, 80, 118, 156, 194, 232, 270, 308, 346, 384, 422, 460, 498, 536, 574, 612, 650, 688, and 726, with up to 1, 2, 3, 4, 5, or 6 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H1 of SEQ ID NO:1, a CDR-H2 of SEQ ID NO:2, a CDR-H3 of SEQ ID NO:3, a CDR-L1 of SEQ ID NO:4, a CDR-L2 of SEQ ID NO:5, and a CDR-L1 of SEQ ID NO:6, as determined by the Exemplary numbering system.

In some embodiments, an antibody provided herein comprises a CDR-H1 of SEQ ID NO:39, a CDR-H2 of SEQ ID NO:40, a CDR-H3 of SEQ ID NO:41, a CDR-L1 of SEQ ID NO:42, a CDR-L2 of SEQ ID NO:43, and a CDR-L1 of SEQ ID NO:44, as determined by the Exemplary numbering system.

In some embodiments, an antibody provided herein comprises a CDR-H1 of SEQ ID NO:77, a CDR-H2 of SEQ ID NO:78, a CDR-H3 of SEQ ID NO:79, a CDR-L1 of SEQ ID NO:80, a CDR-L2 of SEQ ID NO:81, and a CDR-L1 of SEQ ID NO:82, as determined by the Exemplary numbering system.

In some embodiments, an antibody provided herein comprises a CDR-H1 of SEQ ID NO:115, a CDR-H2 of SEQ ID NO:116, a CDR-H3 of SEQ ID NO:117, a CDR-L1 of SEQ ID NO: 118, a CDR-L2 of SEQ ID NO: 119, and a CDR-L1 of SEQ ID NO:120, as determined by the Exemplary numbering system.

In some embodiments, an antibody provided herein comprises a CDR-H1 of SEQ ID NO:153, a CDR-H2 of SEQ ID NO:154, a CDR-H3 of SEQ ID NO:155, a CDR-L1 of SEQ ID NO:156, a CDR-L2 of SEQ ID NO:157, and a CDR-L1 of SEQ ID NO:158, as determined by the Exemplary numbering system.

In some embodiments, an antibody provided herein comprises a CDR-H1 of SEQ ID NO:884, a CDR-H2 of SEQ ID NO:885, a CDR-H3 of SEQ ID NO:886, a CDR-L1 of SEQ ID NO:887, a CDR-L2 of SEQ ID NO:888, and a CDR-L1 of SEQ ID NO:889, as determined by the Exemplary numbering system.

In some embodiments, an antibody provided herein comprises a CDR-H1 of SEQ ID NO:191, a CDR-H2 of SEQ ID NO:192, a CDR-H3 of SEQ ID NO:193, a CDR-L1 of SEQ ID NO:194, a CDR-L2 of SEQ ID NO:195, and a CDR-L1 of SEQ ID NO:196, as determined by the Exemplary numbering system.

In some embodiments, an antibody provided herein comprises a CDR-H1 of SEQ ID NO:229, a CDR-H2 of SEQ ID NO:230, a CDR-H3 of SEQ ID NO:231, a CDR-L1 of SEQ ID NO:232, a CDR-L2 of SEQ ID NO:233, and a CDR-L1 of SEQ ID NO:234, as determined by the Exemplary numbering system.

In some embodiments, an antibody provided herein comprises a CDR-H1 of SEQ ID NO:267, a CDR-H2 of SEQ ID NO:268, a CDR-H3 of SEQ ID NO:269, a CDR-L1 of SEQ ID NO:270, a CDR-L2 of SEQ ID NO:271, and a CDR-L1 of SEQ ID NO:272, as determined by the Exemplary numbering system.

In some embodiments, an antibody provided herein comprises a CDR-H1 of SEQ ID NO:305, a CDR-H2 of SEQ ID NO:306, a CDR-H3 of SEQ ID NO:307, a CDR-L1 of SEQ ID NO:308, a CDR-L2 of SEQ ID NO:309, and a CDR-L1 of SEQ ID NO:310, as determined by the Exemplary numbering system.

In some embodiments, an antibody provided herein comprises a CDR-H1 of SEQ ID NO:343, a CDR-H2 of SEQ ID NO:344, a CDR-H3 of SEQ ID NO:345, a CDR-L1 of SEQ ID NO:346, a CDR-L2 of SEQ ID NO:347, and a CDR-L1 of SEQ ID NO:348, as determined by the Exemplary numbering system.

In some embodiments, an antibody provided herein comprises a CDR-H1 of SEQ ID NO:381, a CDR-H2 of SEQ ID NO:382, a CDR-H3 of SEQ ID NO:383, a CDR-L1 of SEQ ID NO:384, a CDR-L2 of SEQ ID NO:385, and a CDR-L1 of SEQ ID NO:386, as determined by the Exemplary numbering system.

In some embodiments, an antibody provided herein comprises a CDR-H1 of SEQ ID NO:419, a CDR-H2 of SEQ ID NO:420, a CDR-H3 of SEQ ID NO:421, a CDR-L1 of SEQ ID NO:422, a CDR-L2 of SEQ ID NO:423, and a CDR-L1 of SEQ ID NO:424, as determined by the Exemplary numbering system.

In some embodiments, an antibody provided herein comprises a CDR-H1 of SEQ ID NO:457, a CDR-H2 of SEQ ID NO:458, a CDR-H3 of SEQ ID NO:459, a CDR-L1 of SEQ ID NO:460, a CDR-L2 of SEQ ID NO:461, and a CDR-L1 of SEQ ID NO:462, as determined by the Exemplary numbering system.

In some embodiments, an antibody provided herein comprises a CDR-H1 of SEQ ID NO:495, a CDR-H2 of SEQ ID NO:496, a CDR-H3 of SEQ ID NO:497, a CDR-L1 of SEQ ID NO:498, a CDR-L2 of SEQ ID NO:499, and a CDR-L1 of SEQ ID NO:500, as determined by the Exemplary numbering system.

In some embodiments, an antibody provided herein comprises a CDR-H1 of SEQ ID NO:533, a CDR-H2 of SEQ ID NO:534, a CDR-H3 of SEQ ID NO:535, a CDR-L1 of SEQ ID NO:536, a CDR-L2 of SEQ ID NO:537, and a CDR-L1 of SEQ ID NO:538, as determined by the Exemplary numbering system.

In some embodiments, an antibody provided herein comprises a CDR-H1 of SEQ ID NO:571, a CDR-H2 of SEQ ID NO:572, a CDR-H3 of SEQ ID NO:573, a CDR-L1 of SEQ ID NO:574, a CDR-L2 of SEQ ID NO:575, and a CDR-L1 of SEQ ID NO:576, as determined by the Exemplary numbering system.

In some embodiments, an antibody provided herein comprises a CDR-H1 of SEQ ID NO:609, a CDR-H2 of SEQ ID NO:610, a CDR-H3 of SEQ ID NO:611, a CDR-L1 of SEQ ID NO:612, a CDR-L2 of SEQ ID NO:613, and a CDR-L1 of SEQ ID NO:614, as determined by the Exemplary numbering system.

In some embodiments, an antibody provided herein comprises a CDR-H1 of SEQ ID NO:647, a CDR-H2 of SEQ ID NO:648, a CDR-H3 of SEQ ID NO:649, a CDR-L1 of SEQ ID NO:650, a CDR-L2 of SEQ ID NO:651, and a CDR-L1 of SEQ ID NO:652, as determined by the Exemplary numbering system.

In some embodiments, an antibody provided herein comprises a CDR-H1 of SEQ ID NO:685, a CDR-H2 of SEQ ID NO:686, a CDR-H3 of SEQ ID NO:687, a CDR-L1 of SEQ ID NO:688, a CDR-L2 of SEQ ID NO:689, and a CDR-L1 of SEQ ID NO:690, as determined by the Exemplary numbering system.

In some embodiments, an antibody provided herein comprises a CDR-H1 of SEQ ID NO:723, a CDR-H2 of SEQ ID NO:724, a CDR-H3 of SEQ ID NO:725, a CDR-L1 of SEQ ID NO:726, a CDR-L2 of SEQ ID NO:727, and a CDR-L1 of SEQ ID NO:728, as determined by the Exemplary numbering system.

2.2.5. Consensus Sequences

In some embodiments, provided herein is a first family of antibodies, wherein an antibody of such family comprises the following six CDR sequences: (a) a CDR-H1 having the sequence G-F-T-F-S-X₁-Y-A-M-X₂, wherein X₁ is D or S and X₂ is A or G (SEQ ID NO:773); (b) a CDR-H2 having the sequence X₃-I-S-G-S-G-G-L-T-Y-Y-A-D-S-V-K-G, wherein X₃ is A or T (SEQ ID NO:774); (c) a CDR-H3 having the sequence APYGYYMDV (SEQ ID NO:775); (d) a CDR-L1 having the sequence RASQSISSWLA (SEQ ID NO:776); (e) a CDR-L2 having the sequence KASSLES (SEQ ID NO:777); and (f) a CDR-L3 having the sequence QQYKSYIT (SEQ ID NO:778). In some embodiments, an antibody of such family comprises a V_{H} sequence of SEQ ID NO:761 and a V_{L} sequence of SEQ ID NO:762. In some embodiments, provided herein is an antibody within such first family.

In some embodiments, provided herein is a second family of antibodies, wherein an antibody of such family comprises the following six CDR sequences: (a) a CDR-H1 having the sequence G-Y-T-F-X₁-X₂-Y-G-I-S, wherein X₁ is D or R and X₂ is S or V (SEQ ID NO:779); (b) a CDR-H2 having the sequence W-X₃-A-P-Y-X₄-G-N-T-N-Y-A-Q-K-L-Q-G, wherein X₃ is I or V and X₄ is S or N (SEQ ID NO:780); (c) a CDR-H3 having the sequence D-A-G-T-Y-S-P-X₅-G-Y-G-M-D-V, wherein X₅ is F or Y (SEQ ID NO:781); (d) a CDR-L1 having the sequence X₆-A-S-X₇-S-I-X₈-X₉-W-L-A, wherein X₆ is R or Q, X₇ is Q, E, or H, X₈ is S, D, or N, and X₉ is S or N (SEQ ID NO:782); (e) a CDR-L2 having the sequence X₁₀-A-X₁₁-X₁₂-L-E-X₁₃, wherein X₁₀ is K or S, X₁₁ is S or Y, X₁₂ is S, Y, or N, and X₁₃ is S or Y (SEQ ID NO:783); and (f) a CDR-L3 having the sequence Q-X₁₄-F-Q-X₁₅-L-P-P-F-T, wherein X₁₄ is Q, L, or R, and X₁₅ is S or K (SEQ ID NO:784). In some embodiments, an antibody of such family comprises a V_{H} sequence of SEQ ID NO:763 and a V_{L} sequence of SEQ ID NO:764. In some embodiments, provided herein is an antibody within such second family.

In some embodiments, provided herein is a third family of antibodies, wherein an antibody of such family comprises the following six CDR sequences: (a) a CDR-H1 having the sequence G-F-T-F-X₁-S-X₂-G-M-H, wherein X₁ is H or R and X₂ is R or Y (SEQ ID NO:785); (b) a CDR-H2 having the sequence VITYDGINKYYADSVEG (SEQ ID NO:786); (c) a CDR-H3 having the sequence DGVYYGVYDY (SEQ ID NO:787); (d) a CDR-L1 having the sequence KSSQSVLFSSNNKNYLA (SEQ ID NO:788); (e) a CDR-L2 having the sequence WASTRES (SEQ ID NO:789); and (f) a CDR-L3 having the sequence QQFHSYPLT (SEQ ID NO:790). In some embodiments, an antibody of such family comprises a V_{H} sequence of SEQ ID NO:765 and a V_{L} sequence of SEQ ID NO:766. In some embodiments, provided herein is an antibody within such third family.

In some embodiments, provided herein is a fourth family of antibodies, wherein an antibody of such family comprises the following six CDR sequences: (a) a CDR-H1 having the sequence GGTFSSNAIG (SEQ ID NO:791); (b) a CDR-H2 having the sequence SIIPIIGFANYAQKFQG (SEQ ID NO:792); (c) a CDR-H3 having the sequence DSGYYYGASSFGMDV (SEQ ID NO:793); (d) a CDR-L1 having the sequence RASQSVSSNLA (SEQ ID NO:794); (e) a CDR-L2 having the sequence GASTRAT (SEQ ID NO:795); and (f) a CDR-L3 having the sequence EQYNNLPLT (SEQ ID NO:796). In some embodiments, an antibody of such family comprises a V_{H} sequence of SEQ ID NO:767 and a V_{L} sequence of SEQ ID NO:768. In some embodiments, provided herein is an antibody within such fourth family.

In some embodiments, provided herein is a fifth family of antibodies, wherein an antibody of such family comprises the following six CDR sequences: (a) a CDR-H1 having the sequence G-G-S-X₁-S-S-G-X₂-Y-W-S, wherein X₁ is I or L and X₂ is Q or Y (SEQ ID NO:797); (b) a CDR-H2 having the sequence E-I-X₃-X₄-S-G-S-T-R-Y-N-P-S-L-K-S, wherein X₃ is Y or G and X₄ is Y or A (SEQ ID NO:798); (c) a CDR-H3 having the sequence D-X₅-P-Y-Y-Y-X₆-G-G-Y-Y-Y-Y-M-D-V, wherein X₅ is T or A and X₆ is E, G, or D (SEQ ID NO:799); (d) a CDR-L1 having the sequence R-A-S-X₇-S-V-X₈-S-S-X₉-L-A, wherein X₇ is Q, E, or D, X₈ is S or D, and X₉ is Y or F (SEQ ID NO:800); (e) a CDR-L2 having the sequence G-A-X₁₀-X₁₁-R-X₁₂-X₁₃, wherein X₁₀ is S, D, F, or Y, X₁₁ is S or T, X₁₂ is A or Q, and X₁₃ is T or N (SEQ ID NO:801); and (f) a CDR-L3 having the sequence Q-Q-X₁₄-G-V-V-P-Y-T, wherein X₁₄ is V, A, or D (SEQ ID NO:802). In some embodiments, an antibody of such family comprises a V_{H} sequence of SEQ ID NO:769 and a V_{L} sequence of SEQ ID NO:770. In some embodiments, provided herein is an antibody within such fifth family.

In some embodiments, provided herein is a sixth family of antibodies, wherein an antibody of such family comprises the following six CDR sequences: (a) a CDR-H1 having the sequence GYTFANYYMH (SEQ ID NO:803); (b) a CDR-H2 having the sequence IINPSGGITVYAQKFQG (SEQ ID NO:804); (c) a CDR-H3 having the sequence GGSKVAALAFDI (SEQ ID NO:805); (d) a CDR-L1 having the sequence QASQDISNSLN (SEQ ID NO:806); (e) a CDR-L2 having the sequence DASNLET (SEQ ID NO:807); and (f) a CDR-L3 having the sequence QQYNFHPLT (SEQ ID NO:808). In some embodiments, an antibody of such family comprises a V_{H} sequence of SEQ ID NO:771 and a V_{L} sequence of SEQ ID NO:772. In some embodiments, provided herein is an antibody within such sixth family.

In some embodiments, provided herein is a seventh family of antibodies, wherein an antibody of such family comprises the following six CDR sequences: (a) a CDR-H1 having the sequence G-Y-T-F-D-X₁-Y-G-I-S, wherein X₁ is V or A (SEQ ID NO:872); (b) a CDR-H2 having the sequence W-I-A-P-Y-X₂-G-N-T-N-Y-A-Q-K-L-Q-G, wherein X₂ is N or S (SEQ ID NO:873); (c) a CDR-H3 having the sequence D-A-G-T-Y-S-P-F-G-Y-G-M-D-V (SEQ ID NO:874); (d) a CDR-L1 having the sequence X₃-A-S-X₄-S-I-X₅-X₆-W-L-A, wherein X₃ is R or Q, X₄ is Q or E, X₅ is S or N, and X₆ is S or N (SEQ ID NO:875); (e) a CDR-L2 having the sequence K-A-X₇-X₈-L-E-X₉, wherein X₇ is S or Y, X₈ is S or N, and X₉ is S or Y (SEQ ID NO:876); and (f) a CDR-L3 having the sequence Q-X₁₀-F-Q-X₁₁-L-P-P-F-T, wherein X₁₀ is Q or L, and X₁₁ is S or K (SEQ ID NO:877). In some embodiments, an antibody of such family comprises a V_{H} sequence of SEQ ID NO:868 and a V_{L} sequence of SEQ ID NO:869. In some embodiments, provided herein is an antibody within such seventh family.

In some embodiments, provided herein is an eighth family of antibodies, wherein an antibody of such family comprises the following six CDR sequences: (a) a CDR-H1 having the sequence G-Y-T-F-R-S-Y-G-I-S(SEQ ID NO:878); (b) a CDR-H2 having the sequence W-V-A-P-Y-X₁-G-N-T-N-Y-A-Q-K-L-Q-G, wherein X₁ is S or N (SEQ ID NO:879); (c) a CDR-H3 having the sequence D-A-G-T-Y-S-P-Y-G-Y-G-M-D-V (SEQ ID NO:880); (d) a CDR-L1 having the sequence X₂-A-S-X₃-S-I-X₄-S-W-L-A, wherein X₂ is R or Q, X₃ is Q or H, X₄ is S or D (SEQ ID NO:881); (e) a CDR-L2 having the sequence X₅-A-S-X₆-L-E-S, wherein X₅ is K or S, X₆ is S or Y (SEQ ID NO:882); and (f) a CDR-L3 having the sequence Q-X₇-F-Q-S-L-P-P-F-T, wherein X₇ is Q, L, or R (SEQ ID NO:883). In some embodiments, an antibody of such family comprises a V_{H} sequence of SEQ ID NO:870 and a V_{L} sequence of SEQ ID NO:871. In some embodiments, provided herein is an antibody within such eighth family.

2.2.6. Functional Properties of Antibody Variants

As described above, and elsewhere in this disclosure, provided herein are antibody variants defined based on percent identity to an illustrative antibody sequence provided herein, or substitution of amino acid residues in comparison to an illustrative antibody sequence provided herein.

In some embodiments, a variant of an antibody provided herein has specificity for hTF. In some embodiments, a variant of an antibody provided herein has specificity for cTF. In some embodiments, a variant of an antibody provided herein has specificity for mTF. In some embodiments, a variant of an antibody provided herein has specificity for hTF and cTF. In some embodiments, a variant of an antibody provided herein has specificity for hTF and mTF. In some embodiments, a variant of an antibody provided herein has specificity for cTF and mTF. In some embodiments, a variant of an antibody provided herein has specificity for hTF, cTF and mTF.

In some embodiments, a variant of an antibody that is derived from an illustrative antibody sequence provided herein retains affinity, as measured by K_{D}, for hTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for cTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for mTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for both hTF and cTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for both hTF and mTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for both cTF and mTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for all three of hTF, cTF and mTF that is within about 1.5-fold, about 2-fold, about 3-fold, about 4-fold, about 5-fold, about 6-fold, about 7-fold, about 8-fold, about 9-fold or about 10-fold the affinity of such illustrative antibody.

In some embodiments, a variant of an antibody provided herein retains the ability to inhibit TF signaling, as measured by one or more assays or biological effects described herein. In some embodiments, a variant of an antibody provided herein retains the normal function of TF in the blood coagulation processes.

In some embodiments, a variant of an antibody provided herein competes for binding to TF with an antibody selected from 1F, 1G, 25A, 25A3, 25A5, 25A5-T, 25G, 25G1, 25G9, 29D, 29E, 39A, 43B, 43B1, 43B7, 43D, 43D7, 43D8, 43E, 43Ea, and 54E, each as provided in Table 13 of this disclosure. In some embodiments, a variant of an antibody provided herein competes for binding to TF with an antibody selected from 25A, 25A3, 25A5, 25A5-T, 25G, 25G1, and 25G9. In some embodiments, a variant of an antibody provided herein competes for binding to TF with an antibody selected from 43B, 43B1, 43B7, 43D, 43D7, 43D8, 43E, and 43Ea. In some embodiments, a variant of an antibody provided herein competes for binding to TF with an antibody selected from 25A, 25A3, 25A5, 25A5-T, 25G, 25G1, 25G9, 43B, 43B1, 43B7, 43D, 43D7, 43D8, 43E, and 43Ea. In some embodiments, a variant of an antibody provided herein competes for binding to TF with an antibody selected from 1F, 1G, 29D, 29E, 39A, or 54E.

In some embodiments, a variant of an antibody provided herein allows human thrombin generation as determined by thrombin generation assay (TGA). In some embodiments, a variant of an antibody provided herein does not inhibit human thrombin generation as determined by thrombin generation assay (TGA).

In some embodiments, a variant of an antibody provided herein binds human TF at a human TF binding site that is distinct from a human TF binding site bound by human FX. In some embodiments, a variant of an antibody provided herein does not interfere with the ability of TF:FVIIa to convert FX into FXa.

In some embodiments, a variant of an antibody provided herein binds human TF at a human TF binding site that is distinct from a human TF binding site bound by human FVIIa. In some embodiments, a variant of an antibody provided herein does not compete for binding to human TF with human FVIIa.

In some embodiments, a variant of an antibody provided herein inhibits FVIIa-dependent TF signaling.

In some embodiments, a variant of an antibody provided herein binds mouse TF (SEQ ID NO:817). In some embodiments, a variant of an antibody provided herein binds mouse TF with an affinity lower (as indicated by higher K_{D}) than the affinity of the antibody for hTF. In some embodiments, a variant of an antibody provided herein does not bind mTF.

In some embodiments, a variant of an antibody provided herein binds pig TF (SEQ ID NO:824). In some embodiments, a variant of an antibody provided herein binds pig TF with an affinity lower (as indicated by higher K_{D}) than the affinity of the antibody for hTF. In some embodiments, a variant of an antibody provided herein does not bind pTF.

In some embodiments, a variant of an antibody provided herein binds the same epitope of TF as such antibody.

2.2.7. Other Functional Properties of Antibodies

In some embodiments, an antibody provided herein has one or more of the characteristics listed in the following (a)-(dd): (a) binds human TF at a human TF binding site that is distinct from a human TF binding site bound by human FVIIa; (b) does not inhibit human thrombin generation as determined by thrombin generation assay (TGA); (c) does not reduce the thrombin peak on a thrombin generation curve (Peak IIa) compared to an isotype control; (d) does not increase the time from the assay start to the thrombin peak on a thrombin generation curve (ttPeak) compared to an isotype control; (e) does not decrease the endogenous thrombin potential (ETP) as determined by the area under a thrombin generation curve compared to an isotype control; (f) allows human thrombin generation as determined by thrombin generation assay (TGA); (g) maintains the thrombin peak on a thrombin generation curve (Peak IIa) compared to an isotype control; (h) maintains the time from the assay start to the thrombin peak on a thrombin generation curve (ttPeak) compared to an isotype control; (i) preserves the endogenous thrombin potential (ETP) as determined by the area under a thrombin generation curve compared to an isotype control; (j) binds human TF at a human TF binding site that is distinct from a human TF binding site bound by human FX; (k) does not interfere with the ability of TF:FVIIa to convert FX into FXa; (1) does not compete for binding to human TF with human FVIIa; (m) inhibits FVIIa-dependent TF signaling; (n) binds to cynomolgus TF; (o) binds to mouse TF; (p) binds to rabbit TF; (q) binds to pig TF; (r) reduces lesion size in a swine choroidal neovascularization (CNV) model; (s) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a variant TF extracellular domain comprising a mutation at amino acid residue 68 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (bb) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (cc) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (dd) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay. In some embodiments, an antibody provided herein has two or more of the characteristics listed in the foregoing (a)-(dd). In some embodiments, an antibody provided herein has three or more of the characteristics listed in the foregoing (a)-(dd). In some embodiments, an antibody provided herein has four or more of the characteristics listed in the foregoing (a)-(dd). In some embodiments, an antibody provided herein has five or more of the characteristics listed in the foregoing (a)-(dd). In some embodiments, an antibody provided herein has six or more of the characteristics listed in the foregoing (a)-(dd). In some embodiments, an antibody provided herein has seven or more of the characteristics listed in the foregoing (a)-(dd). In some embodiments, an antibody provided herein has eight or more of the characteristics listed in the foregoing (a)-(dd). In some embodiments, an antibody provided herein has nine or more of the characteristics listed in the foregoing (a)-(dd). In some embodiments, an antibody provided herein has ten or more of the characteristics listed in the foregoing (a)-(dd). In some embodiments, an antibody provided herein has eleven or more of the characteristics listed in the foregoing (a)-(dd). In some embodiments, an antibody provided herein has twelve or more of the characteristics listed in the foregoing (a)-(dd). In some embodiments, an antibody provided herein has thirteen or more of the characteristics listed in the foregoing (a)-(dd). In some embodiments, an antibody provided herein has fourteen or more of the characteristics listed in the foregoing (a)-(dd). In some embodiments, an antibody provided herein has fifteen or more of the characteristics listed in the foregoing (a)-(dd). In some embodiments, an antibody provided herein has sixteen or more of the characteristics listed in the foregoing (a)-(dd). In some embodiments, an antibody provided herein has seventeen or more of the characteristics listed in the foregoing (a)-(dd). In some embodiments, an antibody provided herein has eighteen or more of the characteristics listed in the foregoing (a)-(dd). In some embodiments, an antibody provided herein has nineteen or more of the characteristics listed in the foregoing (a)-(dd). In some embodiments, an antibody provided herein has twenty or more of the characteristics listed in the foregoing (a)-(dd). In some embodiments, an antibody provided herein has twenty-one or more of the characteristics listed in the foregoing (a)-(dd). In some embodiments, an antibody provided herein has twenty-two or more of the characteristics listed in the foregoing (a)-(dd). In some embodiments, an antibody provided herein has twenty-three of the characteristics listed in the foregoing (a)-(dd). In some embodiments, an antibody provided herein has twenty-four of the characteristics listed in the foregoing (a)-(dd). In some embodiments, an antibody provided herein has twenty-five of the characteristics listed in the foregoing (a)-(dd). In some embodiments, an antibody provided herein has twenty-six of the characteristics listed in the foregoing (a)-(dd). In some embodiments, an antibody provided herein has twenty-seven of the characteristics listed in the foregoing (a)-(dd). In some embodiments, an antibody provided herein has twenty-eight of the characteristics listed in the foregoing (a)-(dd). In some embodiments, an antibody provided herein has twenty-nine of the characteristics listed in the foregoing (a)-(dd). In some embodiments, an antibody provided herein has all thirty of the characteristics listed in the foregoing (a)-(dd).

In some embodiments, an antibody provided herein has one or more of the characteristics listed in the following (a)-(dd): (a) binds human TF at a human TF binding site that is distinct from a human TF binding site bound by human FVIIa; (b) does not inhibit human thrombin generation as determined by thrombin generation assay (TGA); (c) does not reduce the thrombin peak on a thrombin generation curve (Peak IIa) compared to an isotype control; (d) does not increase the time from the assay start to the thrombin peak on a thrombin generation curve (ttPeak) compared to an isotype control; (e) does not decrease the endogenous thrombin potential (ETP) as determined by the area under a thrombin generation curve compared to an isotype control; (f) allows human thrombin generation as determined by thrombin generation assay (TGA); (g) maintains the thrombin peak on a thrombin generation curve (Peak IIa) compared to an isotype control; (h) maintains the time from the assay start to the thrombin peak on a thrombin generation curve (ttPeak) compared to an isotype control; (i) preserves the endogenous thrombin potential (ETP) as determined by the area under a thrombin generation curve compared to an isotype control; (j) binds human TF at a human TF binding site that is distinct from a human TF binding site bound by human FX; (k) does not interfere with the ability of TF:FVIIa to convert FX into FXa; (1) does not compete for binding to human TF with human FVIIa; (m) inhibits FVIIa-dependent TF signaling; (n) binds to cynomolgus TF; (o) binds to mouse TF; (p) binds to rabbit TF; (q) binds to pig TF; (r) reduces lesion size in a swine choroidal neovascularization (CNV) model; (s) the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a variant TF extracellular domain comprising a mutation K68N of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (bb) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (cc) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (dd) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay. In some embodiments, an antibody provided herein has two or more of the characteristics listed in the foregoing (a)-(dd). In some embodiments, an antibody provided herein has three or more of the characteristics listed in the foregoing (a)-(dd). In some embodiments, an antibody provided herein has four or more of the characteristics listed in the foregoing (a)-(dd). In some embodiments, an antibody provided herein has five or more of the characteristics listed in the foregoing (a)-(dd). In some embodiments, an antibody provided herein has six or more of the characteristics listed in the foregoing (a)-(dd). In some embodiments, an antibody provided herein has seven or more of the characteristics listed in the foregoing (a)-(dd). In some embodiments, an antibody provided herein has eight or more of the characteristics listed in the foregoing (a)-(dd). In some embodiments, an antibody provided herein has nine or more of the characteristics listed in the foregoing (a)-(dd). In some embodiments, an antibody provided herein has ten or more of the characteristics listed in the foregoing (a)-(dd). In some embodiments, an antibody provided herein has eleven or more of the characteristics listed in the foregoing (a)-(dd). In some embodiments, an antibody provided herein has twelve or more of the characteristics listed in the foregoing (a)-(dd). In some embodiments, an antibody provided herein has thirteen or more of the characteristics listed in the foregoing (a)-(dd). In some embodiments, an antibody provided herein has fourteen or more of the characteristics listed in the foregoing (a)-(dd). In some embodiments, an antibody provided herein has fifteen or more of the characteristics listed in the foregoing (a)-(dd). In some embodiments, an antibody provided herein has sixteen or more of the characteristics listed in the foregoing (a)-(dd). In some embodiments, an antibody provided herein has seventeen or more of the characteristics listed in the foregoing (a)-(dd). In some embodiments, an antibody provided herein has eighteen or more of the characteristics listed in the foregoing (a)-(dd). In some embodiments, an antibody provided herein has nineteen or more of the characteristics listed in the foregoing (a)-(dd). In some embodiments, an antibody provided herein has twenty or more of the characteristics listed in the foregoing (a)-(dd). In some embodiments, an antibody provided herein has twenty-one or more of the characteristics listed in the foregoing (a)-(dd). In some embodiments, an antibody provided herein has twenty-two or more of the characteristics listed in the foregoing (a)-(dd). In some embodiments, an antibody provided herein has twenty-three of the characteristics listed in the foregoing (a)-(dd). In some embodiments, an antibody provided herein has twenty-four of the characteristics listed in the foregoing (a)-(dd). In some embodiments, an antibody provided herein has twenty-five of the characteristics listed in the foregoing (a)-(dd). In some embodiments, an antibody provided herein has twenty-six of the characteristics listed in the foregoing (a)-(dd). In some embodiments, an antibody provided herein has twenty-seven of the characteristics listed in the foregoing (a)-(dd). In some embodiments, an antibody provided herein has twenty-eight of the characteristics listed in the foregoing (a)-(dd). In some embodiments, an antibody provided herein has twenty-nine of the characteristics listed in the foregoing (a)-(dd). In some embodiments, an antibody provided herein has all thirty of the characteristics listed in the foregoing (a)-(dd).

In some embodiments, an antibody provided herein exhibits a combination of characteristics comprising two or more of characteristics listed in the following (a)-(dd): (a) binds human TF at a human TF binding site that is distinct from a human TF binding site bound by human FVIIa; (b) does not inhibit human thrombin generation as determined by thrombin generation assay (TGA); (c) does not reduce the thrombin peak on a thrombin generation curve (Peak IIa) compared to an isotype control; (d) does not increase the time from the assay start to the thrombin peak on a thrombin generation curve (ttPeak) compared to an isotype control; (e) does not decrease the endogenous thrombin potential (ETP) as determined by the area under a thrombin generation curve compared to an isotype control; (f) allows human thrombin generation as determined by thrombin generation assay (TGA); (g) maintains the thrombin peak on a thrombin generation curve (Peak IIa) compared to an isotype control; (h) maintains the time from the assay start to the thrombin peak on a thrombin generation curve (ttPeak) compared to an isotype control; (i) preserves the endogenous thrombin potential (ETP) as determined by the area under a thrombin generation curve compared to an isotype control; (j) binds human TF at a human TF binding site that is distinct from a human TF binding site bound by human FX; (k) does not interfere with the ability of TF:FVIIa to convert FX into FXa; (1) does not compete for binding to human TF with human FVIIa; (m) inhibits FVIIa-dependent TF signaling; (n) binds to cynomolgus TF; (o) binds to mouse TF; (p) binds to rabbit TF; (q) binds to pig TF; (r) reduces lesion size in a swine choroidal neovascularization (CNV) model; (s) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a variant TF extracellular domain comprising a mutation at amino acid residue 68 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (bb) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (cc) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (dd) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of characteristics comprising two or more of characteristics listed in the following (a)-(dd): (a) binds human TF at a human TF binding site that is distinct from a human TF binding site bound by human FVIIa; (b) does not inhibit human thrombin generation as determined by thrombin generation assay (TGA); (c) does not reduce the thrombin peak on a thrombin generation curve (Peak IIa) compared to an isotype control; (d) does not increase the time from the assay start to the thrombin peak on a thrombin generation curve (ttPeak) compared to an isotype control; (e) does not decrease the endogenous thrombin potential (ETP) as determined by the area under a thrombin generation curve compared to an isotype control; (f) allows human thrombin generation as determined by thrombin generation assay (TGA); (g) maintains the thrombin peak on a thrombin generation curve (Peak IIa) compared to an isotype control; (h) maintains the time from the assay start to the thrombin peak on a thrombin generation curve (ttPeak) compared to an isotype control; (i) preserves the endogenous thrombin potential (ETP) as determined by the area under a thrombin generation curve compared to an isotype control; (j) binds human TF at a human TF binding site that is distinct from a human TF binding site bound by human FX; (k) does not interfere with the ability of TF:FVIIa to convert FX into FXa; (1) does not compete for binding to human TF with human FVIIa; (m) inhibits FVIIa-dependent TF signaling; (n) binds to cynomolgus TF; (o) binds to mouse TF; (p) binds to rabbit TF; (q) binds to pig TF; (r) reduces lesion size in a swine choroidal neovascularization (CNV) model; (s) the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a variant TF extracellular domain comprising a mutation K68N of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (bb) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (cc) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (dd) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); binds to cynomolgus TF; the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); binds to cynomolgus TF; the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); binds to cynomolgus TF; the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); binds to cynomolgus TF; the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

2.3. Affinity and Other Properties of TF Antibodies

2.3.1. Affinity of TF Antibodies

In some embodiments, the affinity of an antibody provided herein for TF as indicated by K_{D}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. In some embodiments, the affinity of the antibody is between about 10⁻⁷ M and 10⁻¹² M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸ M and 10⁻¹² M. In some embodiments, the affinity of the antibody is between about 10⁻⁸ M and 10⁻¹¹ M. In some embodiments, the affinity of the antibody is between about 10⁻⁹ M and 10⁻¹¹ M. In some embodiments, the affinity of the antibody is between about 10⁻¹⁰ M and 10⁻¹¹ M.

In some embodiments, the K_{D} value of an antibody provided herein for cTF is no more than 15× of the K_{D} value of the antibody for hTF. In some embodiments, the K_{D} value of an antibody provided herein for cTF is no more than 10× of the K_{D} value of the antibody for hTF. In some embodiments, the K_{D} value of an antibody provided herein for cTF is no more than 8× of the K_{D} value of the antibody for hTF. In some embodiments, the K_{D} value of an antibody provided herein for cTF is no more than 5× of the K_{D} value of the antibody for hTF. In some embodiments, the K_{D} value of an antibody provided herein for cTF is no more than 3× of the K_{D} value of the antibody for hTF. In some embodiments, the K_{D} value of an antibody provided herein for cTF is no more than 2× of the K_{D} value of the antibody for hTF.

In some embodiments, the K_{D} value of an antibody provided herein for mTF is no more than 20× of the K_{D} value of the antibody for hTF. In some embodiments, the K_{D} value of an antibody provided herein for mTF is no more than 15× of the K_{D} value of the antibody for hTF. In some embodiments, the K_{D} value of an antibody provided herein for mTF is no more than 10× of the K_{D} value of the antibody for hTF. In some embodiments, the K_{D} value of an antibody provided herein for mTF is no more than 5× of the K_{D} value of the antibody for hTF. In some embodiments, the K_{D} value of an antibody provided herein for mTF is no more than 2× of the K_{D} value of the antibody for hTF.

In some embodiments, the affinity of an antibody provided herein for hTF as indicated by K_{D} measured by Biacore, as set forth in Table 5 is selected from about 0.31 nM, about 6.20 nM, about 0.36 nM, about 0.08 nM, about 23.0 nM, about 0.94 nM, about 13.3 nM, about 0.47 nM, about 0.09 nM, about 1.75 nM, about 0.07 nM, about 0.14 nM, about 2.09 nM, about 0.06 nM, about 0.15 nM, about 1.46 nM, about 1.60 nM, and about 0.42 nM. In some embodiments, such affinity as indicated by K_{D} ranges from about 23.0 nM to about 0.06 nM. In some embodiments, such is about 23.0 nM or less.

In some embodiments, the affinity of an antibody provided herein for hTF as indicated by K_{D} measured by ForteBio, as set forth in Table 5 is selected from about 1.28 nM, about 2.20 nM, about 8.45 nM, about 1.67 nM, about 0.64 nM, about 21.9 nM, about 3.97 nM, about 35.8 nM, about 3.30 nM, about 2.32 nM, about 0.83 nM, about 2.40 nM, about 0.96 nM, about 0.86 nM, about 3.84 nM, about 1.02 nM, about 1.61 nM, about 2.52 nM, about 2.28 nM, and about 1.59 nM. In some embodiments, such affinity as indicated by K_{D} ranges from about 35.8 nM to about 0.64 nM. In some embodiments, such K_{D} is about 35.8 nM or less.

In some embodiments, the affinity of an antibody provided herein for cTF as indicated by K_{D} measured by Biacore, as set forth in Table 5 is selected from about 0.26 nM, about 5.42 nM, about 0.21 nM, about 0.04 nM, about 18.0 nM, about 0.78 nM, about 16.4 nM, about 5.06 nM, about 0.08 nM, about 5.64 nM, about 0.12 nM, about 0.24 nM, about 5.66 nM, about 0.39 nM, about 5.69 nM, about 6.42 nM, and about 1.83 nM. In some embodiments, such affinity as indicated by K_{D} ranges from about 18.0 nM to about 0.04 nM. In some embodiments, such K_{D} is about 18.0 nM or less.

In some embodiments, the affinity of an antibody provided herein for cTF as indicated by K_{D} measured by ForteBio, as set forth in Table 5 is selected from about 1.43 nM, about 2.70 nM, about 7.65 nM, about 1.36 nM, about 0.76 nM, about 17.5 nM, about 4.99 nM, about 42.9 nM, about 12.0 nM, about 15.0 nM, about 0.57 nM, about 3.40 nM, about 1.05 nM, about 0.94 nM, about 4.12 nM, about 1.11 nM, about 1.96 nM, about 4.07 nM, about 2.71 nM, and about 4.16 nM. In some embodiments, such affinity as indicated by K_{D} ranges from about 42.9 nM to about 0.57 nM. In some embodiments, such K_{D} is about 42.9 nM or less.

In some embodiments, the affinity of an antibody provided herein for mTF as indicated by K_{D} measured by Biacore, as set forth in Table 5 is selected from about 5.4 nM, about 2.9 nM, about 21 nM, and about 2.4 nM. In some embodiments, such affinity as indicated by K_{D} ranges from about 21 nM to about 2.4 nM. In some embodiments, such K_{D} is about 21 nM or less.

In some embodiments, the affinity of an antibody provided herein for mTF as indicated by K_{D} measured by ForteBio, as set forth in Table 5 is selected from about 263 nM, about 131 nM, about 188 nM, about 114 nM, about 34.2 nM, about 9.16 nM, about 161 nM, about 72.1 nM, about 360 nM, about 281 nM, about 41.4 nM, about 6.12 nM, about 121 nM, and about 140 nM. In some embodiments, such affinity as indicated by K_{D} ranges from about 360 nM to about 6.12 nM. In some embodiments, such K_{D} is about 360 nM or less.

In some embodiments, the affinity of an antibody provided herein for hTF as indicated by EC₅₀ measured with human TF-positive HCT-116 cells, (as set forth in international PCT application PCT/US2019/012427 and U.S. utility application Ser. No. 16/959,652, incorporated herein by reference in their entirety) is selected from about 50 pM, about 58 pM, about 169 pM, about 77 pM, about 88 pM, about 134 pM, about 85 pM, about 237 pM, about 152 pM, about 39 pM, about 559 pM, about 280 pM, about 255 pM, about 147 pM, about 94 pM, about 117 pM, about 687 pM, about 532 pM, and about 239 pM. In some embodiments, such affinity ranges from about 687 pM to about 39 pM. In some embodiments, such EC₅₀ is about 687 pM or less.

In some embodiments, the affinity of an antibody provided herein for mTF as indicated by EC₅₀ measured with mouse TF-positive CHO cells, (as set forth in international PCT application PCT/US2019/012427 and U.S. utility application Ser. No. 16/959,652, incorporated herein by reference in their entirety) is selected from about 455 nM, about 87 nM, about 11 nM, about 3.9 nM, about 3.0 nM, about 3.4 nM, about 255 nM, about 2.9 nM, about 3.6 nM, and about 4.0 nM. In some embodiments, such affinity ranges from about 455 nM to about 2.9 nM. In some embodiments, such EC₅₀ is about 455 pM or less.

In some embodiments, the K_{D} value of an antibody provided herein for pTF is no more than 20× of the K_{D} value of the antibody for hTF. In some embodiments, the K_{D} value of an antibody provided herein for pTF is no more than 15× of the K_{D} value of the antibody for hTF. In some embodiments, the K_{D} value of an antibody provided herein for pTF is no more than 10× of the K_{D} value of the antibody for hTF. In some embodiments, the K_{D} value of an antibody provided herein for pTF is no more than 5× of the K_{D} value of the antibody for hTF. In some embodiments, the K_{D} value of an antibody provided herein for pTF is no more than 2× of the K_{D} value of the antibody for hTF.

In some embodiments, the affinity of an antibody provided herein for pTF as indicated by K_{D} measured by Biacore, as set forth in Table 40 is 3.31 nM or 12.9 nM.

2.3.2. Thrombin Generation in the Presence of TF Antibodies

In some embodiments, the TF antibodies provided herein do not inhibit human thrombin generation as determined by thrombin generation assay (TGA). In certain embodiments, the TF antibodies provided herein allow human thrombin generation as determined by thrombin generation assay (TGA).

In some embodiments, the percent peak thrombin generation (% Peak IIa) is at least 40% in the presence of no less than 100 nM TF antibody compared to the control conditions without the antibody, as determined by thrombin generation assay (TGA). In some embodiments, the % Peak IIa is at least 50% in the presence of no less than 100 nM TF antibody compared to the control conditions without the antibody, as determined by thrombin generation assay (TGA). In some embodiments, the % Peak IIa is at least 60% in the presence of no less than 100 nM TF antibody compared to the control conditions without the antibody, as determined by thrombin generation assay (TGA). In some embodiments, the % Peak IIa is at least 70% in the presence of no less than 100 nM TF antibody compared to the control conditions without the antibody, as determined by thrombin generation assay (TGA). In some embodiments, the % Peak IIa is at least 80% in the presence of no less than 100 nM TF antibody compared to the control conditions without the antibody, as determined by thrombin generation assay (TGA). In some embodiments, the % Peak IIa is at least 90% in the presence of no less than 100 nM TF antibody compared to the control conditions without the antibody, as determined by thrombin generation assay (TGA). In some embodiments, the % Peak IIa is at least 95% in the presence of no less than 100 nM TF antibody compared to the control conditions without the antibody, as determined by thrombin generation assay (TGA). In some embodiments, the % Peak IIa is at least 99% in the presence of no less than 100 nM TF antibody compared to the control conditions without the antibody, as determined by thrombin generation assay (TGA).

In some embodiments, the % Peak IIa is at least 40% in the presence of no less than 50 nM TF antibody compared to the control conditions without the antibody, as determined by thrombin generation assay (TGA). In some embodiments, the % Peak IIa is at least 50% in the presence of no less than 50 nM TF antibody compared to the control conditions without the antibody, as determined by thrombin generation assay (TGA). In some embodiments, the % Peak IIa is at least 60% in the presence of no less than 50 nM TF antibody compared to the control conditions without the antibody, as determined by thrombin generation assay (TGA). In some embodiments, the % Peak IIa is at least 70% in the presence of no less than 50 nM TF antibody compared to the control conditions without the antibody, as determined by thrombin generation assay (TGA). In some embodiments, the % Peak IIa is at least 80% in the presence of no less than 50 nM TF antibody compared to the control conditions without the antibody, as determined by thrombin generation assay (TGA). In some embodiments, the % Peak IIa is at least 90% in the presence of no less than 50 nM TF antibody compared to the control conditions without the antibody, as determined by thrombin generation assay (TGA). In some embodiments, the % Peak IIa is at least 95% in the presence of no less than 50 nM TF antibody compared to the control conditions without the antibody, as determined by thrombin generation assay (TGA). In some embodiments, the % Peak IIa is at least 99% in the presence of no less than 50 nM TF antibody compared to the control conditions without the antibody, as determined by thrombin generation assay (TGA).

In some embodiments, the % Peak IIa is at least 60% in the presence of no less than 10 nM TF antibody compared to the control conditions without the antibody, as determined by thrombin generation assay (TGA). In some embodiments, the % Peak IIa is at least 70% in the presence of no less than 10 nM TF antibody compared to the control conditions without the antibody, as determined by thrombin generation assay (TGA). In some embodiments, the % Peak IIa is at least 80% in the presence of no less than 10 nM TF antibody compared to the control conditions without the antibody, as determined by thrombin generation assay (TGA). In some embodiments, the % Peak IIa is at least 90% in the presence of no less than 10 nM TF antibody compared to the control conditions without the antibody, as determined by thrombin generation assay (TGA). In some embodiments, the % Peak IIa is at least 95% in the presence of no less than 10 nM TF antibody compared to the control conditions without the antibody, as determined by thrombin generation assay (TGA). In some embodiments, the % Peak IIa is at least 99% in the presence of no less than 10 nM TF antibody compared to the control conditions without the antibody, as determined by thrombin generation assay (TGA).

In some embodiments, the % Peak IIa in the presence of 100 nM TF antibody, as set forth in Table 6 and Table 37 is selected from about 99%, about 100%, about 103%, about 64%, about 52%, about 87%, about 96%, about 98%, and about 53% compared to the control conditions without the antibody, as determined by thrombin generation assay (TGA) without antibody pre-incubation. In some embodiments, such % Peak IIa ranges from about 52% to about 103%. In some embodiments, such % Peak IIa is about 52% or more.

In some embodiments, the % Peak IIa in the presence of 50 nM TF antibody, as set forth in Table 6 and Table 37 is selected from about 99%, about 100%, about 103%, about 67%, about 58%, about 89%, about 96%, about 98%, about 68%, about 62%, and about 88% compared to the control conditions without the antibody, as determined by thrombin generation assay (TGA) without antibody pre-incubation. In some embodiments, such % Peak IIa ranges from about 58% to about 103%. In some embodiments, such % Peak IIa is about 58% or more.

In some embodiments, the % Peak IIa in the presence of 10 nM TF antibody, as set forth in Table 6 and Table 37 is selected from about 100%, about 99%, about 103%, about 87%, about 83%, about 95%, about 98%, about 86%, and about 96% compared to the control conditions without the antibody, as determined by thrombin generation assay (TGA) without antibody pre-incubation. In some embodiments, such % Peak IIa ranges from about 83% to about 103%. In some embodiments, such % Peak IIa is about 83% or more.

In some embodiments, the % Peak IIa in the presence of 100 nM TF antibody, as set forth in Table 7 and Table 38 is selected from about 108%, about 105%, about 111%, about 58%, about 47%, about 91%, about 103%, about 109%, about 107%, and about 45% compared to the control conditions without the antibody, as determined by thrombin generation assay (TGA) with 10 min antibody pre-incubation. In some embodiments, such % Peak IIa ranges from about 45% to about 111%. In some embodiments, such % Peak IIa is about 45% or more.

In some embodiments, the % Peak IIa in the presence of 50 nM TF antibody, as set forth in Table 7 and Table 38 is selected from about 107%, about 104%, about 114%, about 62%, about 49%, about 87%, about 105%, about 109%, about 55%, and about 92% compared to the control conditions without the antibody, as determined by thrombin generation assay (TGA) with 10 min antibody pre-incubation. In some embodiments, such % Peak IIa ranges from about 49% to about 114%. In some embodiments, such % Peak IIa is about 49% or more.

In some embodiments, the % Peak IIa in the presence of 10 nM TF antibody, as set forth in Table 7 and Table 38 is selected from about 105%, about 114%, about 76%, about 68%, about 94%, about 108%, about 104%, about 74%, and about 93% compared to the control conditions without the antibody, as determined by thrombin generation assay (TGA) with 10 min antibody pre-incubation. In some embodiments, such % Peak IIa ranges from about 68% to about 114%. In some embodiments, such % Peak IIa is about 68% or more.

In some embodiments, the percent endogenous thrombin potential (% ETP) is at least 80% in the presence of no less than 100 nM TF antibody compared to the control conditions without the antibody, as determined by thrombin generation assay (TGA). In some embodiments, the % ETP is at least 90% in the presence of no less than 100 nM TF antibody compared to the control conditions without the antibody, as determined by thrombin generation assay (TGA). In some embodiments, the % ETP is at least 95% in the presence of no less than 100 nM TF antibody compared to the control conditions without the antibody, as determined by thrombin generation assay (TGA). In some embodiments, the % ETP is at least 99% in the presence of no less than 100 nM TF antibody compared to the control conditions without the antibody, as determined by thrombin generation assay (TGA).

In some embodiments, the % ETP is at least 80% in the presence of no less than 50 nM TF antibody compared to the control conditions without the antibody, as determined by thrombin generation assay (TGA). In some embodiments, the % ETP is at least 90% in the presence of no less than 50 nM TF antibody compared to the control conditions without the antibody, as determined by thrombin generation assay (TGA). In some embodiments, the % ETP is at least 95% in the presence of no less than 50 nM TF antibody compared to the control conditions without the antibody, as determined by thrombin generation assay (TGA). In some embodiments, the % ETP is at least 99% in the presence of no less than 50 nM TF antibody compared to the control conditions without the antibody, as determined by thrombin generation assay (TGA).

In some embodiments, the % ETP is at least 80% in the presence of no less than 10 nM TF antibody compared to the control conditions without the antibody, as determined by thrombin generation assay (TGA). In some embodiments, the % ETP is at least 90% in the presence of no less than 10 nM TF antibody compared to the control conditions without the antibody, as determined by thrombin generation assay (TGA). In some embodiments, the % ETP is at least 95% in the presence of no less than 10 nM TF antibody compared to the control conditions without the antibody, as determined by thrombin generation assay (TGA). In some embodiments, the % ETP is at least 99% in the presence of no less than 10 nM TF antibody compared to the control conditions without the antibody, as determined by thrombin generation assay (TGA).

In some embodiments, the % ETP in the presence of 100 nM TF antibody, as set forth in Table 6 and Table 37 is selected from about 108%, about 103%, about 109%, about 100%, about 96%, about 102%, about 105%, and about 92% compared to the control conditions without the antibody, as determined by thrombin generation assay (TGA) without antibody pre-incubation. In some embodiments, such % ETP ranges from about 92% to about 109%. In some embodiments, such % ETP is about 92% or more.

In some embodiments, the % ETP in the presence of 50 nM TF antibody, as set forth in Table 6 and Table 37 is selected from about 108%, about 103%, about 111%, about 101%, about 97%, about 104%, about 106%, about 93%, about 96%, and about 105% compared to the control conditions without the antibody, as determined by thrombin generation assay (TGA) without antibody pre-incubation. In some embodiments, such % ETP ranges from about 93% to about 111%. In some embodiments, such % ETP is about 93% or more.

In some embodiments, the % ETP in the presence of 10 nM TF antibody, as set forth in Table 6 and Table 37 is selected from about 106%, about 109%, about 105%, about 104%, about 107%, about 99%, about 101%, and about 102% compared to the control conditions without the antibody, as determined by thrombin generation assay (TGA) without antibody pre-incubation. In some embodiments, such % ETP ranges from about 99% to about 109%. In some embodiments, such % ETP is about 99% or more.

In some embodiments, the % ETP in the presence of 100 nM TF antibody, as set forth in Table 7 and Table 38 is selected from about 110%, about 104%, about 106%, about 98%, about 95%, about 108%, about 107%, about 96%, about 92%, and about 103% compared to the control conditions without the antibody, as determined by thrombin generation assay (TGA) with 10 min antibody pre-incubation. In some embodiments, such % ETP ranges from about 92% to about 110%. In some embodiments, such % ETP is about 92% or more.

In some embodiments, the % ETP in the presence of 50 nM TF antibody, as set forth in Table 7 and Table 38 is selected from about 110%, about 106%, about 108%, about 103%, about 96%, about 109%, about 102%, about 104%, about 94%, and about 98% compared to the control conditions without the antibody, as determined by thrombin generation assay (TGA) with 10 min antibody pre-incubation. In some embodiments, such % ETP ranges from about 94% to about 110%. In some embodiments, such % ETP is about 94% or more.

In some embodiments, the % ETP in the presence of 10 nM TF antibody, as set forth in Table 7 and Table 38 is selected from about 107%, about 106%, about 110%, about 103%, about 100%, about 105%, about 102%, and about 101% compared to the control conditions without the antibody, as determined by thrombin generation assay (TGA) with 10 min antibody pre-incubation. In some embodiments, such % ETP ranges from about 100% to about 110%. In some embodiments, such % ETP is about 100% or more.

2.3.3. FXa Conversion in the Presence of TF Antibodies

In some embodiments, the antibodies provided herein bind human TF at a human TF binding site that is distinct from a human TF binding site bound by human FX. In certain embodiments, the antibodies provided herein do not interfere with the ability of TF:FVIIa to convert FX into FXa.

In some embodiments, the percentage of FXa conversion (% FXa) is at least 75% in the presence of no less than 100 nM TF antibody compared to the control conditions without the antibody. In some embodiments, the % FXa is at least 80% in the presence of no less than 100 nM TF antibody compared to the control conditions without the antibody. In some embodiments, the % FXa is at least 85% in the presence of no less than 100 nM TF antibody compared to the control conditions without the antibody. In some embodiments, the % FXa is at least 90% in the presence of no less than 100 nM TF antibody compared to the control conditions without the antibody. In some embodiments, the % FXa is at least 95% in the presence of no less than 100 nM TF antibody compared to the control conditions without the antibody.

In some embodiments, the % FXa is at least 75% in the presence of no less than 50 nM TF antibody compared to the control conditions without the antibody. In some embodiments, the % FXa is at least 80% in the presence of no less than 50 nM TF antibody compared to the control conditions without the antibody. In some embodiments, the % FXa is at least 85% in the presence of no less than 50 nM TF antibody compared to the control conditions without the antibody. In some embodiments, the % FXa is at least 90% in the presence of no less than 50 nM TF antibody compared to the control conditions without the antibody. In some embodiments, the % FXa is at least 95% in the presence of no less than 50 nM TF antibody compared to the control conditions without the antibody.

In some embodiments, the % FXa is at least 75% in the presence of no less than 25 nM TF antibody compared to the control conditions without the antibody. In some embodiments, the % FXa is at least 80% in the presence of no less than 25 nM TF antibody compared to the control conditions without the antibody. In some embodiments, the % FXa is at least 85% in the presence of no less than 25 nM TF antibody compared to the control conditions without the antibody. In some embodiments, the % FXa is at least 90% in the presence of no less than 25 nM TF antibody compared to the control conditions without the antibody. In some embodiments, the % FXa is at least 95% in the presence of no less than 25 nM TF antibody compared to the control conditions without the antibody.

In some embodiments, the % FXa is at least 75% in the presence of no less than 12.5 nM TF antibody compared to the control conditions without the antibody. In some embodiments, the % FXa is at least 80% in the presence of no less than 12.5 nM TF antibody compared to the control conditions without the antibody. In some embodiments, the % FXa is at least 85% in the presence of no less than 12.5 nM TF antibody compared to the control conditions without the antibody. In some embodiments, % FXa is at least 90% in the presence of no less than 12.5 nM TF antibody compared to the control conditions without the antibody. In some embodiments, the % FXa is at least 95% in the presence of no less than 12.5 nM TF antibody compared to the control conditions without the antibody.

In some embodiments, the % FXa in the presence of 100 nM TF antibody, as set forth in Table 8 is selected from about 89%, about 96%, about 116%, about 108%, about 117%, about 105%, about 112%, about 106%, about 103%, about 111%, about 98%, and about 101% compared to the control conditions without the antibody. In some embodiments, such % FXa ranges from about 89% to about 117%. In some embodiments, such % FXa is about 89% or more.

In some embodiments, the % FXa in the presence of 50 nM TF antibody, as set forth in Table 8 is selected from about 94%, about 93%, about 78%, about 102%, about 99%, about 104%, about 105%, about 108%, about 107%, about 97%, and about 106% compared to the control conditions without the antibody. In some embodiments, such % FXa ranges from about 78% to about 108%. In some embodiments, such % FXa is about 78% or more.

In some embodiments, the % FXa in the presence of 25 nM TF antibody, as set forth in Table 8 is selected from about 81%, about 89%, about 85%, about 109%, about 96%, about 97%, about 108%, about 104%, about 103%, about 112%, and about 89% compared to the control conditions without the antibody. In some embodiments, such % FXa ranges from about 81% to about 112%. In some embodiments, such % FXa is about 81% or more.

In some embodiments, the % FXa in the presence of 12.5 nM TF antibody, as set forth in Table 8 is selected from about 87%, about 89%, about 82%, about 99%, about 101%, about 98%, about 113%, about 106%, about 115%, about 110%, about 120%, about 85%, and about 108% compared to the control conditions without the antibody. In some embodiments, such % FXa ranges from about 82% to about 120%. In some embodiments, such % FXa is about 82% or more.

2.3.4. FVIIa Binding in the Presence of TF Antibodies

In some embodiments, the antibodies provided herein bind human TF at a human TF binding site that is distinct from a human TF binding site bound by human FVIIa. In certain embodiments, the antibodies provided herein do not compete for binding to human TF with human FVIIa.

In some embodiments, the percentage of FVIIa binding (% FVIIa) is at least 75% in the presence of no less than 250 nM TF antibody compared to the control conditions without the antibody. In some embodiments, the % FVIIa is at least 80% in the presence of no less than 250 nM TF antibody compared to the control conditions without the antibody. In some embodiments, the % FVIIa is at least 85% in the presence of no less than 250 nM TF antibody compared to the control conditions without the antibody. In some embodiments, the % FVIIa is at least 90% in the presence of no less than 250 nM TF antibody compared to the control conditions without the antibody. In some embodiments, the % FVIIa is at least 95% in the presence of no less than 250 nM TF antibody compared to the control conditions without the antibody.

In some embodiments, the % FVIIa is at least 75% in the presence of no less than 83 nM TF antibody compared to the control conditions without the antibody. In some embodiments, the % FVIIa is at least 80% in the presence of no less than 83 nM TF antibody compared to the control conditions without the antibody. In some embodiments, the % FVIIa is at least 85% in the presence of no less than 83 nM TF antibody compared to the control conditions without the antibody. In some embodiments, the % FVIIa is at least 90% in the presence of no less than 83 nM TF antibody compared to the control conditions without the antibody. In some embodiments, the % FVIIa is at least 95% in the presence of no less than 83 nM TF antibody compared to the control conditions without the antibody.

In some embodiments, the % FVIIa is at least 75% in the presence of no less than 28 nM TF antibody compared to the control conditions without the antibody. In some embodiments, the % FVIIa is at least 80% in the presence of no less than 28 nM TF antibody compared to the control conditions without the antibody. In some embodiments, the % FVIIa is at least 85% in the presence of no less than 28 nM TF antibody compared to the control conditions without the antibody. In some embodiments, the % FVIIa is at least 90% in the presence of no less than 28 nM TF antibody compared to the control conditions without the antibody. In some embodiments, the % FVIIa is at least 95% in the presence of no less than 28 nM TF antibody compared to the control conditions without the antibody.

In some embodiments, the % FVIIa is at least 75% in the presence of no less than 9.25 nM TF antibody compared to the control conditions without the antibody. In some embodiments, the % FVIIa is at least 80% in the presence of no less than 9.25 nM TF antibody compared to the control conditions without the antibody. In some embodiments, the % FVIIa is at least 85% in the presence of no less than 9.25 nM TF antibody compared to the control conditions without the antibody. In some embodiments, the % FVIIa is at least 90% in the presence of no less than 9.25 nM TF antibody compared to the control conditions without the antibody. In some embodiments, the % FVIIa is at least 95% in the presence of no less than 9.25 nM TF antibody compared to the control conditions without the antibody.

In some embodiments, the % FVIIa in the presence of 250 nM TF antibody, as set forth in Table 9 is selected from about 98%, about 87%, about 80%, about 92%, about 95%, about 89%, about 91%, about 97%, about 94%, about 101%, and about 96% compared to the control conditions without the antibody. In some embodiments, such % FVIIa ranges from about 80% to about 101%. In some embodiments, such % FVIIa is about 80% or more.

In some embodiments, the % FVIIa in the presence of 83 nM TF antibody, as set forth in Table 9 is selected from about 97%, about 88%, about 77%, about 93%, about 94%, about 91%, about 98%, about 100%, and about 92% compared to the control conditions without the antibody. In some embodiments, such % FVIIa ranges from about 77% to about 100%. In some embodiments, such % FVIIa is about 77% or more.

In some embodiments, the % FVIIa in the presence of 28 nM TF antibody, as set forth in Table 9 is selected from about 101%, about 87%, about 79%, about 96%, about 93%, about 95%, about 98%, about 100%, about 102%, about 99%, about 92%, and about 91% compared to the control conditions without the antibody. In some embodiments, such % FVIIa ranges from about 79% to about 102%. In some embodiments, such % FVIIa is about 79% or more.

In some embodiments, the % FVIIa in the presence of 9.25 nM TF antibody, as set forth in Table 9 is selected from about 100%, about 90%, about 76%, about 97%, about 93%, about 99%, about 98%, about 102%, about 101%, and about 95% compared to the control conditions without the antibody. In some embodiments, such % FVIIa ranges from about 76% to about 102%. In some embodiments, such % FVIIa is about 76% or more.

2.3.5. FVIIa-Dependent TF Signaling in the Presence of TF Antibodies

In some embodiments, the antibodies provided herein inhibit FVIIa-dependent TF signaling. In some embodiments, the inhibition of FVIIa-dependent TF signaling is measured by the reduction of IL8. In some embodiments, the inhibition of FVIIa-dependent TF signaling is measured by the reduction of GM-CSF.

In some embodiments, the Interleukin 8 concentration (IL8 conc) is reduced by at least 70% in the presence of no less than 100 nM TF antibody compared to the control conditions without the antibody. In some embodiments, the IL8 conc is reduced by at least 80% in the presence of no less than 100 nM TF antibody compared to the control conditions without the antibody. In some embodiments, the IL8 conc is reduced by at least 90% in the presence of no less than 100 nM TF antibody compared to the control conditions without the antibody.

In some embodiments, the IL8 conc is reduced by at least 70% in the presence of no less than 40 nM TF antibody compared to the control conditions without the antibody. In some embodiments, the IL8 conc is reduced by at least 80% in the presence of no less than 40 nM TF antibody compared to the control conditions without the antibody. In some embodiments, the IL8 conc is reduced by at least 90% in the presence of no less than 40 nM TF antibody compared to the control conditions without the antibody.

In some embodiments, the IL8 conc is reduced by at least 60% in the presence of no less than 16 nM TF antibody compared to the control conditions without the antibody. In some embodiments, the IL8 conc is reduced by at least 70% in the presence of no less than 16 nM TF antibody compared to the control conditions without the antibody. In some embodiments, the IL8 conc is reduced by at least 80% in the presence of no less than 16 nM TF antibody compared to the control conditions without the antibody. In some embodiments, the IL8 conc is reduced by at least 90% in the presence of no less than 16 nM TF antibody compared to the control conditions without the antibody.

In some embodiments, the IL8 conc is reduced by at least 50% in the presence of no less than 6.4 nM TF antibody compared to the control conditions without the antibody. In some embodiments, the IL8 conc is reduced by at least 60% in the presence of no less than 6.4 nM TF antibody compared to the control conditions without the antibody. In some embodiments, the IL8 conc is reduced by at least 70% in the presence of no less than 6.4 nM TF antibody compared to the control conditions without the antibody. In some embodiments, the IL8 conc is reduced by at least 80% in the presence of no less than 6.4 nM TF antibody compared to the control conditions without the antibody. In some embodiments, the IL8 conc is reduced by at least 90% in the presence of no less than 6.4 nM TF antibody compared to the control conditions without the antibody.

In some embodiments, the Granulocyte-Macrophage Colony-Stimulating Factor concentration (GM-CSF conc) is reduced by at least 70% in the presence of no less than 100 nM TF antibody compared to the control conditions without the antibody. In some embodiments, the GM-CSF conc is reduced by at least 80% in the presence of no less than 100 nM TF antibody compared to the control conditions without the antibody. In some embodiments, the GM-CSF conc is reduced by at least 90% in the presence of no less than 100 nM TF antibody compared to the control conditions without the antibody.

In some embodiments, the GM-CSF conc is reduced by at least 70% in the presence of no less than 40 nM TF antibody compared to the control conditions without the antibody. In some embodiments, the GM-CSF conc is reduced by at least 80% in the presence of no less than 40 nM TF antibody compared to the control conditions without the antibody. In some embodiments, the GM-CSF conc is reduced by at least 90% in the presence of no less than 40 nM TF antibody compared to the control conditions without the antibody.

In some embodiments, the GM-CSF conc is reduced by at least 60% in the presence of no less than 16 nM TF antibody compared to the control conditions without the antibody. In some embodiments, the GM-CSF conc is reduced by at least 70% in the presence of no less than 16 nM TF antibody compared to the control conditions without the antibody. In some embodiments, the GM-CSF conc is reduced by at least 80% in the presence of no less than 16 nM TF antibody compared to the control conditions without the antibody. In some embodiments, the GM-CSF conc is reduced by at least 90% in the presence of no less than 16 nM TF antibody compared to the control conditions without the antibody.

In some embodiments, the GM-CSF conc is reduced by at least 50% in the presence of no less than 6.4 nM TF antibody compared to the control conditions without the antibody. In some embodiments, the GM-CSF conc is reduced by at least 60% in the presence of no less than 6.4 nM TF antibody compared to the control conditions without the antibody. In some embodiments, the GM-CSF conc is reduced by at least 70% in the presence of no less than 6.4 nM TF antibody compared to the control conditions without the antibody. In some embodiments, the GM-CSF conc is reduced by at least 80% in the presence of no less than 6.4 nM TF antibody compared to the control conditions without the antibody. In some embodiments, the GM-CSF conc is reduced by at least 90% in the presence of no less than 6.4 nM TF antibody compared to the control conditions without the antibody.

In some embodiments, the percentage of Interleukin 8 (% IL8) in the presence of 100 nM TF antibody, as set forth in Table 10 is selected from about 2%, about 9%, about 8%, about 6%, about 13%, about 1%, about 3%, about 4%, and about 5% compared to the control conditions without the antibody. In some embodiments, such % IL8 ranges from about 1% to about 13%. In some embodiments, such % IL8 is about 13% or less.

In some embodiments, the % IL8 in the presence of 40 nM TF antibody, as set forth in Table 10 is selected from about 2%, about 8%, about 7%, about 10%, about 14%, about 4%, about 5%, and about 6% compared to the control conditions without the antibody. In some embodiments, such % IL8 ranges from about 2% to about 14%. In some embodiments, such % IL8 is about 14% or less.

In some embodiments, the % IL8 in the presence of 16 nM TF antibody, as set forth in Table 10 is selected from about 2%, about 3%, about 10%, about 8%, about 7%, about 16%, about 9%, about 15%, about 5%, and about 6% compared to the control conditions without the antibody. In some embodiments, such % IL8 ranges from about 2% to about 16%. In some embodiments, such % IL8 is about 16% or less.

In some embodiments, the % IL8 in the presence of 6.4 nM TF antibody, as set forth in Table 10 is selected from about 3%, about 4%, about 11%, about 9%, about 14%, about 22%, about 12%, about 6%, about 5%, about 15%, about 21%, and about 8% compared to the control conditions without the antibody. In some embodiments, such % IL8 ranges from about 3% to about 22%. In some embodiments, such % IL8 is about 22% or less.

In some embodiments, the percentage of Granulocyte-Macrophage Colony-Stimulating Factor (% GM-CSF) in the presence of 100 nM TF antibody, as set forth in Table 11 is selected from about 6%, about 7%, about 22%, about 20%, about 12%, about 19%, about 17%, about 25%, about 5%, about 14%, about 11%, and about 10% compared to the control conditions without the antibody. In some embodiments, such % GM-CSF ranges from about 5% to about 25%. In some embodiments, such % GM-CSF is about 25% or less.

In some embodiments, the % GM-CSF in the presence of 40 nM TF antibody, as set forth in Table 11 is selected from about 6%, about 7%, about 19%, about 15%, about 18%, about 16%, about 26%, about 5%, about 13%, about 11%, and about 10% compared to the control conditions without the antibody. In some embodiments, such % GM-CSF ranges from about 5% to about 26%. In some embodiments, such % GM-CSF is about 26% or less.

In some embodiments, the % GM-CSF in the presence of 16 nM TF antibody, as set forth in Table 11 is selected from about 6%, about 7%, about 22%, about 19%, about 14%, about 32%, about 17%, about 26%, about 5%, about 12%, about 13%, about 9%, about 11%, and about 15% compared to the control conditions without the antibody. In some embodiments, such % GM-CSF ranges from about 5% to about 32%. In some embodiments, such % GM-CSF is about 32% or less.

In some embodiments, the % GM-CSF in the presence of 6.4 nM TF antibody, as set forth in Table 11 is selected from about 8%, about 9%, about 24%, about 20%, about 18%, about 39%, about 34%, about 15%, about 21%, about 16%, about 17%, and about 10% compared to the control conditions without the antibody. In some embodiments, such % GM-CSF ranges from about 8% to about 39%. In some embodiments, such % GM-CSF is about 39% or less.

2.3.6. Lesion Size Reduction in Swine Choroidal Neovascularization (CNV) Model

In some embodiments, the antibodies provided herein reduce lesion size in a swine choroidal neovascularization (CNV) model. In some embodiments, the reduction in lesion size is measured by Fluorescein Angiography (FA).

In some embodiments, the lesion size in a swine CNV model is reduced by at least 5% 7 days after administration of the anti-TF antibody. In some embodiments, the lesion size in a swine CNV model is reduced by at least 10% 7 days after administration of the anti-TF antibody. In some embodiments, the lesion size in a swine CNV model is reduced by at least 20% 7 days after administration of the anti-TF antibody. In some embodiments, the lesion size in a swine CNV model is reduced by at least 40% 7 days after administration of the anti-TF antibody. In some embodiments, the lesion size in a swine CNV model is reduced by at least 60% 7 days after administration of the anti-TF antibody.

In some embodiments, the lesion size in a swine CNV model is reduced by at least 10% 21 days after administration of the anti-TF antibody. In some embodiments, the lesion size in a swine CNV model is reduced by at least 20% 21 days after administration of the anti-TF antibody. In some embodiments, the lesion size in a swine CNV model is reduced by at least 40% 21 days after administration of the anti-TF antibody. In some embodiments, the lesion size in a swine CNV model is reduced by at least 60% 21 days after administration of the anti-TF antibody. In some embodiments, the lesion size in a swine CNV model is reduced by at least 80% 21 days after administration of the anti-TF antibody.

2.4. Germlines

The antibodies provided herein may comprise any suitable V_{H} and V_{L} germline sequences.

In some embodiments, the V_{H} region of an antibody provided herein is from the VH3 germline. In some embodiments, the V_{H} region of an antibody provided herein is from the VH1 germline. In some embodiments, the V_{H} region of an antibody provided herein is from the VH4 germline.

In some embodiments, the V_{H} region of an antibody provided herein is from the VH3-23 germline. In some embodiments, the V_{H} region of an antibody provided herein is from the VH1-18 germline. In some embodiments, the V_{H} region of an antibody provided herein is from the VH3-30 germline. In some embodiments, the V_{H} region of an antibody provided herein is from the VH1-69 germline. In some embodiments, the V_{H} region of an antibody provided herein is from the VH4-31 germline. In some embodiments, the V_{H} region of an antibody provided herein is from the VH4-34 germline. In some embodiments, the V_{H} region of an antibody provided herein is from the VH1-46 germline.

In some embodiments, the V_{L} region of an antibody provided herein is from the VK1 germline. In some embodiments, the V_{L} region of an antibody provided herein is from the VK4 germline. In some embodiments, the V_{L} region of an antibody provided herein is from the VK3 germline

In some embodiments, the V_{L} region of an antibody provided herein is from the VK1-05 germline. In some embodiments, the V_{L} region of an antibody provided herein is from the VK4-01 germline. In some embodiments, the V_{L} region of an antibody provided herein is from the VK3-15 germline. In some embodiments, the V_{L} region of an antibody provided herein is from the VK3-20 germline. In some embodiments, the V_{L} region of an antibody provided herein is from the VK1-33 germline.

2.5. Monospecific and Multispecific TF Antibodies

In some embodiments, the antibodies provided herein are monospecific antibodies.

In some embodiments, the antibodies provided herein are multispecific antibodies.

In some embodiments, a multispecific antibody provided herein binds more than one antigen. In some embodiments, a multispecific antibody binds two antigens. In some embodiments, a multispecific antibody binds three antigens. In some embodiments, a multispecific antibody binds four antigens. In some embodiments, a multispecific antibody binds five antigens.

In some embodiments, a multispecific antibody provided herein binds more than one epitope on a TF antigen. In some embodiments, a multispecific antibody binds two epitopes on a TF antigen. In some embodiments, a multispecific antibody binds three epitopes on a TF antigen.

Many multispecific antibody constructs are known in the art, and the antibodies provided herein may be provided in the form of any suitable multispecific suitable construct.

In some embodiments, the multispecific antibody comprises an immunoglobulin comprising at least two different heavy chain variable regions each paired with a common light chain variable region (i.e., a “common light chain antibody”). The common light chain variable region forms a distinct antigen-binding domain with each of the two different heavy chain variable regions. See Merchant et al., Nature Biotechnol., 1998, 16:677-681, incorporated by reference in its entirety.

In some embodiments, the multispecific antibody comprises an immunoglobulin comprising an antibody or fragment thereof attached to one or more of the N- or C-termini of the heavy or light chains of such immunoglobulin. See Coloma and Morrison, Nature Biotechnol., 1997, 15:159-163, incorporated by reference in its entirety. In some aspects, such antibody comprises a tetravalent bispecific antibody.

In some embodiments, the multispecific antibody comprises a hybrid immunoglobulin comprising at least two different heavy chain variable regions and at least two different light chain variable regions. See Milstein and Cuello, Nature, 1983, 305:537-540; and Staerz and Bevan, Proc. Natl. Acad. Sci. USA, 1986, 83:1453-1457; each of which is incorporated by reference in its entirety.

In some embodiments, the multispecific antibody comprises immunoglobulin chains with alterations to reduce the formation of side products that do not have multispecificity. In some aspects, the antibodies comprise one or more “knobs-into-holes” modifications as described in U.S. Pat. No. 5,731,168, incorporated by reference in its entirety.

In some embodiments, the multispecific antibody comprises immunoglobulin chains with one or more electrostatic modifications to promote the assembly of Fc hetero-multimers. See WO 2009/089004, incorporated by reference in its entirety.

In some embodiments, the multispecific antibody comprises a bispecific single chain molecule. See Traunecker et al., EMBO J., 1991, 10:3655-3659; and Gruber et al., J. Immunol., 1994, 152:5368-5374; each of which is incorporated by reference in its entirety.

In some embodiments, the multispecific antibody comprises a heavy chain variable domain and a light chain variable domain connected by a polypeptide linker, where the length of the linker is selected to promote assembly of multispecific antibodies with the desired multispecificity. For example, monospecific scFvs generally form when a heavy chain variable domain and light chain variable domain are connected by a polypeptide linker of more than 12 amino acid residues. See U.S. Pat. Nos. 4,946,778 and 5,132,405, each of which is incorporated by reference in its entirety. In some embodiments, reduction of the polypeptide linker length to less than 12 amino acid residues prevents pairing of heavy and light chain variable domains on the same polypeptide chain, thereby allowing pairing of heavy and light chain variable domains from one chain with the complementary domains on another chain. The resulting antibodies therefore have multispecificity, with the specificity of each binding site contributed by more than one polypeptide chain. Polypeptide chains comprising heavy and light chain variable domains that are joined by linkers between 3 and 12 amino acid residues form predominantly dimers (termed diabodies). With linkers between 0 and 2 amino acid residues, trimers (termed triabodies) and tetramers (termed tetrabodies) are favored. However, the exact type of oligomerization appears to depend on the amino acid residue composition and the order of the variable domain in each polypeptide chain (e.g., V_{H}-linker-V_{L} vs. V_{L}-linker-V_{H}), in addition to the linker length. A skilled person can select the appropriate linker length based on the desired multispecificity.

In some embodiments, the multispecific antibody comprises a diabody. See Hollinger et al., Proc. Natl. Acad. Sci. USA, 1993, 90:6444-6448, incorporated by reference in its entirety. In some embodiments, the multispecific antibody comprises a triabody. See Todorovska et al., J. Immunol. Methods, 2001, 248:47-66, incorporated by reference in its entirety. In some embodiments, the multispecific antibody comprises a tetrabody. See id., incorporated by reference in its entirety.

In some embodiments, the multispecific antibody comprises a trispecific F(ab′)3 derivative. See Tutt et al. J. Immunol., 1991, 147:60-69, incorporated by reference in its entirety.

In some embodiments, the multispecific antibody comprises a cross-linked antibody. See U.S. Pat. No. 4,676,980; Brennan et al., Science, 1985, 229:81-83; Staerz, et al. Nature, 1985, 314:628-631; and EP 0453082; each of which is incorporated by reference in its entirety.

In some embodiments, the multispecific antibody comprises antigen-binding domains assembled by leucine zippers. See Kostelny et al., J. Immunol., 1992, 148:1547-1553, incorporated by reference in its entirety.

In some embodiments, the multispecific antibody comprises complementary protein domains. In some aspects, the complementary protein domains comprise an anchoring domain (AD) and a dimerization and docking domain (DDD). In some embodiments, the AD and DDD bind to each other and thereby enable assembly of multispecific antibody structures via the “dock and lock” (DNL) approach. Antibodies of many specificities may be assembled, including bispecific antibodies, trispecific antibodies, tetraspecific antibodies, quintspecific antibodies, and hexaspecific antibodies. Multispecific antibodies comprising complementary protein domains are described, for example, in U.S. Pat. Nos. 7,521,056; 7,550,143; 7,534,866; and 7,527,787; each of which is incorporated by reference in its entirety.

In some embodiments, the multispecific antibody comprises a dual action Fab (DAF) antibody as described in U.S. Pat. Pub. No. 2008/0069820, incorporated by reference in its entirety.

In some embodiments, the multispecific antibody comprises an antibody formed by reduction of two parental molecules followed by mixing of the two parental molecules and reoxidation to assembly a hybrid structure. See Carlring et al., PLoS One, 2011, 6:e22533, incorporated by reference in its entirety.

In some embodiments, the multispecific antibody comprises a DVD-Ig™. A DVD-Ig™ is a dual variable domain immunoglobulin that can bind to two or more antigens. DVD-Igs™ are described in U.S. Pat. No. 7,612,181, incorporated by reference in its entirety.

In some embodiments, the multispecific antibody comprises a DART™. DART™ are described in Moore et al., Blood, 2011, 117:454-451, incorporated by reference in its entirety.

In some embodiments, the multispecific antibody comprises a DuoBody®. DuoBodies® are described in Labrijn et al., Proc. Natd. Acad. Sci. USA, 2013, 110:5145-5150; Gramer et al., mAbs, 2013, 5:962-972; and Labrijn et al., Nature Protocols, 2014, 9:2450-2463; each of which is incorporated by reference in its entirety.

In some embodiments, the multispecific antibody comprises an antibody fragment attached to another antibody or fragment. The attachment can be covalent or non-covalent. When the attachment is covalent, it may be in the form of a fusion protein or via a chemical linker. Illustrative examples of multispecific antibodies comprising antibody fragments attached to other antibodies include tetravalent bispecific antibodies, where an scFv is fused to the C-terminus of the C_{H3} from an IgG. See Coloma and Morrison, Nature Biotechnol., 1997, 15:159-163. Other examples include antibodies in which a Fab molecule is attached to the constant region of an immunoglobulin. See Miler et al., J. Immunol., 2003, 170:4854-4861, incorporated by reference in its entirety. Any suitable fragment may be used, including any of the fragments described herein or known in the art.

In some embodiments, the multispecific antibody comprises a CovX-Body. CovX-Bodies are described, for example, in Doppalapudi et al., Proc. Natl. Acad. Sci. USA, 2010, 107:22611-22616, incorporated by reference in its entirety.

In some embodiments, the multispecific antibody comprises an Fcab antibody, where one or more antigen-binding domains are introduced into an Fc region. Fcab antibodies are described in Wozniak-Knopp et al., Protein Eng. Des. Sel., 2010, 23:289-297, incorporated by reference in its entirety.

In some embodiments, the multispecific antibody comprises a TandAb® antibody. TandAb® antibodies are described in Kipriyanov et al., J. Mol. Biol., 1999, 293:41-56 and Zhukovsky et al., Blood, 2013, 122:5116, each of which is incorporated by reference in its entirety.

In some embodiments, the multispecific antibody comprises a tandem Fab. Tandem Fabs are described in WO 2015/103072, incorporated by reference in its entirety.

In some embodiments, the multispecific antibody comprises a Zybody™. Zybodies™ are described in LaFleur et al., mAbs, 2013, 5:208-218, incorporated by reference in its entirety.

2.6. Glycosylation Variants

In certain embodiments, an antibody provided herein may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to or from an antibody provided herein may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

In some embodiments, an antibody provided herein comprises a glycosylation motif that is different from a naturally occurring antibody. Any suitable naturally occurring glycosylation motif can be modified in the antibodies provided herein. The structural and glycosylation properties of immunoglobulins, for example, are known in the art and summarized, for example, in Schroeder and Cavacini, J. Allergy Clin. Immunol., 2010, 125:S41-52, incorporated by reference in its entirety.

In some embodiments, an antibody provided herein comprises an IgG1 Fc region with modification to the oligosaccharide attached to asparagine 297 (Asn 297). Naturally occurring IgG1 antibodies produced by mammalian cells typically comprise a branched, biantennary oligosaccharide that is generally attached by an N-linkage to Asn 297 of the C_{H2} domain of the Fc region. See Wright et al., TIBTECH, 1997, 15:26-32, incorporated by reference in its entirety. The oligosaccharide attached to Asn 297 may include various carbohydrates such as mannose, N-acetyl glucosamine (GlcNAc), galactose, and sialic acid, as well as a fucose attached to a GlcNAc in the “stem” of the biantennary oligosaccharide structure.

In some embodiments, the oligosaccharide attached to Asn 297 is modified to create antibodies having altered ADCC. In some embodiments, the oligosaccharide is altered to improve ADCC. In some embodiments, the oligosaccharide is altered to reduce ADCC.

In some aspects, an antibody provided herein comprises an IgG1 domain with reduced fucose content at position Asn 297 compared to a naturally occurring IgG1 domain. Such Fc domains are known to have improved ADCC. See Shields et al., J. Biol. Chem., 2002, 277:26733-26740, incorporated by reference in its entirety. In some aspects, such antibodies do not comprise any fucose at position Asn 297. The amount of fucose may be determined using any suitable method, for example as described in WO 2008/077546, incorporated by reference in its entirety.

In some embodiments, an antibody provided herein comprises a bisected oligosaccharide, such as a biantennary oligosaccharide attached to the Fc region of the antibody that is bisected by GlcNAc. Such antibody variants may have reduced fucosylation and/or improved ADCC function. Examples of such antibody variants are described, for example, in WO 2003/011878; U.S. Pat. No. 6,602,684; and U.S. Pat. Pub. No. 2005/0123546; each of which is incorporated by reference in its entirety.

Other illustrative glycosylation variants which may be incorporated into the antibodies provided herein are described, for example, in U.S. Pat. Pub. Nos. 2003/0157108, 2004/0093621, 2003/0157108, 2003/0115614, 2002/0164328, 2004/0093621, 2004/0132140, 2004/0110704, 2004/0110282, 2004/0109865; International Pat. Pub. Nos. 2000/61739, 2001/29246, 2003/085119, 2003/084570, 2005/035586, 2005/035778; 2005/053742, 2002/031140; Okazaki et al., J. Mol. Biol., 2004, 336:1239-1249; and Yamane-Ohnuki et al., Biotech. Bioeng., 2004, 87: 614-622; each of which is incorporated by reference in its entirety.

In some embodiments, an antibody provided herein comprises an Fc region with at least one galactose residue in the oligosaccharide attached to the Fc region. Such antibody variants may have improved CDC function. Examples of such antibody variants are described, for example, in WO 1997/30087; WO 1998/58964; and WO 1999/22764; each of which is incorporated by reference in its entirety.

Examples of cell lines capable of producing defucosylated antibodies include Lec13 CHO cells, which are deficient in protein fucosylation (see Ripka et al., Arch. Biochem. Biophys., 1986, 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al., Biotech. Bioeng., 2004, 87: 614-622; Kanda et al., Biotechnol. Bioeng., 2006, 94:680-688; and WO 2003/085107; each of which is incorporated by reference in its entirety).

In some embodiments, an antibody provided herein is an aglycosylated antibody. An aglycosylated antibody can be produced using any method known in the art or described herein. In some aspects, an aglycosylated antibody is produced by modifying the antibody to remove all glycosylation sites. In some aspects, the glycosylation sites are removed only from the Fc region of the antibody. In some aspects, an aglycosylated antibody is produced by expressing the antibody in an organism that is not capable of glycosylation, such as E. coli, or by expressing the antibody in a cell-free reaction mixture.

In some embodiments, an antibody provided herein has a constant region with reduced effector function compared to a native IgG1 antibody. In some embodiments, the affinity of a constant region of an Fc region of an antibody provided herein for Fc receptor is less than the affinity of a native IgG1 constant region for such Fc receptor.

2.7. Fc Region Amino Acid Sequence Variants

In certain embodiments, an antibody provided herein comprises an Fc region with one or more amino acid substitutions, insertions, or deletions in comparison to a naturally occurring Fc region. In some aspects, such substitutions, insertions, or deletions yield antibodies with altered stability, glycosylation, or other characteristics. In some aspects, such substitutions, insertions, or deletions yield aglycosylated antibodies.

In some aspects, the Fc region of an antibody provided herein is modified to yield an antibody with altered affinity for an Fc receptor, or an antibody that is more immunologically inert. In some embodiments, the antibody variants provided herein possess some, but not all, effector functions. Such antibodies may be useful, for example, when the half-life of the antibody is important in vivo, but when certain effector functions (e.g., complement activation and ADCC) are unnecessary or deleterious.

In some embodiments, the Fc region of an antibody provided herein is a human IgG4 Fc region comprising one or more of the hinge stabilizing mutations S228P and L235E. See Aalberse et al., Immunology, 2002, 105:9-19, incorporated by reference in its entirety. In some embodiments, the IgG4 Fc region comprises one or more of the following mutations: E233P, F234V, and L235A. See Armour et al., Mol. Immunol., 2003, 40:585-593, incorporated by reference in its entirety. In some embodiments, the IgG4 Fc region comprises a deletion at position G236.

In some embodiments, the Fc region of an antibody provided herein is a human IgG1 Fc region comprising one or more mutations to reduce Fc receptor binding. In some aspects, the one or more mutations are in residues selected from S228 (e.g., S228A), L234 (e.g., L234A), L235 (e.g., L235A), D265 (e.g., D265A), and N297 (e.g., N297A). In some aspects, the antibody comprises a PVA236 mutation. PVA236 means that the amino acid sequence ELLG (SEQ ID NO: 928), from amino acid position 233 to 236 of IgG1 or EFLG (SEQ ID NO: 929) of IgG4, is replaced by PVA. See U.S. Pat. No. 9,150,641, incorporated by reference in its entirety.

In some embodiments, the Fc region of an antibody provided herein is modified as described in Armour et al., Eur. J. Immunol., 1999, 29:2613-2624; WO 1999/058572; and/or U.K. Pat. App. No. 98099518; each of which is incorporated by reference in its entirety.

In some embodiments, the Fc region of an antibody provided herein is a human IgG2 Fc region comprising one or more of mutations A330S and P331S.

In some embodiments, the Fc region of an antibody provided herein has an amino acid substitution at one or more positions selected from 238, 265, 269, 270, 297, 327 and 329. See U.S. Pat. No. 6,737,056, incorporated by reference in its entirety. Such Fc mutants include Fc mutants with substitutions at two or more of amino acid positions 265, 269, 270, 297 and 327, including the so-called “DANA” Fc mutant with substitution of residues 265 and 297 with alanine. See U.S. Pat. No. 7,332,581, incorporated by reference in its entirety. In some embodiments, the antibody comprises an alanine at amino acid position 265. In some embodiments, the antibody comprises an alanine at amino acid position 297.

In certain embodiments, an antibody provided herein comprises an Fc region with one or more amino acid substitutions which improve ADCC, such as a substitution at one or more of positions 298, 333, and 334 of the Fc region. In some embodiments, an antibody provided herein comprises an Fc region with one or more amino acid substitutions at positions 239, 332, and 330, that result in enhanced effector function, as described in Lazar et al., Proc. Natl. Acad. Sci. USA, 2006,103:4005-4010, incorporated by reference in its entirety.

In some embodiments, an antibody provided herein comprises one or more alterations that improves or diminishes C1q binding and/or CDC. See U.S. Pat. No. 6,194,551; WO 99/51642; and Idusogie et al., J. Immunol., 2000, 164:4178-4184; each of which is incorporated by reference in its entirety.

In some embodiments, an antibody provided herein comprises one or more alterations to increase half-life. Antibodies with increased half-lives and improved binding to the neonatal Fc receptor (FcRn) are described, for example, in Hinton et al., J. Immunol., 2006, 176:346-356; and U.S. Pat. Pub. No. 2005/0014934; each of which is incorporated by reference in its entirety. Such Fc variants include those with substitutions at one or more of Fc region residues: 238, 250, 256, 265, 272, 286, 303, 305, 307, 311, 312, 314, 317, 340, 356, 360, 362, 376, 378, 380, 382, 413, 424, 428, and 434 of an IgG.

In some embodiments, an antibody provided herein comprises one or more Fc region variants as described in U.S. Pat. Nos. 7,371,826, 5,648,260, and 5,624,821; Duncan and Winter, Nature, 1988, 322:738-740; and WO 94/29351; each of which is incorporated by reference in its entirety.

2.8. Pyroglutamate

As is known in the art, both glutamate (E) and glutamine (Q) at the N-termini of recombinant proteins can cyclize spontaneously to form pyroglutamate (pE) in vitro and in vivo. See Liu et al., J. Biol. Chem., 2011, 286:11211-11217, incorporated by reference in its entirety.

In some embodiments, provided herein are antibodies comprising a polypeptide sequence having a pE residue at the N-terminal position. In some embodiments, provided herein are antibodies comprising a polypeptide sequence in which the N-terminal residue has been converted from Q to pE. In some embodiments, provided herein are antibodies comprising a polypeptide sequence in which the N-terminal residue has been converted from E to pE.

2.9. Cysteine Engineered Antibody Variants

In certain embodiments, provided herein are cysteine engineered antibodies, also known as “thioMAbs,” in which one or more residues of the antibody are substituted with cysteine residues. In particular embodiments, the substituted residues occur at solvent accessible sites of the antibody. By substituting such residues with cysteine, reactive thiol groups are introduced at solvent accessible sites of the antibody and may be used to conjugate the antibody to other moieties, such as drug moieties or linker-drug moieties, for example, to create an immunoconjugate.

In certain embodiments, any one or more of the following residues may be substituted with cysteine: V205 of the light chain; Al 18 of the heavy chain Fc region; and S400 of the heavy chain Fc region. Cysteine engineered antibodies may be generated as described, for example, in U.S. Pat. No. 7,521,541, which is incorporated by reference in its entirety.

3. Anti-TF Antibody-Drug Conjugates

Provided herein are antibody-drug conjugates (ADCs) comprising an antibody that binds specifically to TF and a cytotoxic agent. In some embodiments, the cytotoxic agent is linked directly to the anti-TF antibody. In some embodiments, the cytotoxic agent is linked indirectly to the anti-TF antibody.

In some embodiments, the ADCs further comprise a linker. In some embodiments, the linker links the anti-TF antibody to the cytotoxic agent.

In some embodiments, the ADCs provided herein have a drug-antibody ratio (DAR) of 1. In some embodiments, the ADCs provided herein have a DAR of 2. In some embodiments, the ADCs provided herein have a DAR of 3. In some embodiments, the ADCs provided herein have a DAR of 4. In some embodiments, the ADCs provided herein have a DAR of 5. In some embodiments, the ADCs provided herein have a DAR of 1-2, 1-3, 1-4, 1-5, 2-3, 2-4, 2-5, 3-4, 3-5, 4-5, 1, 2, 3, 4, or 5. In some embodiments, the ADCs provided herein have a DAR greater than 5. In some embodiments, the DAR is measured by UV/vis spectroscopy, hydrophobic interaction chromatography (HIC), and/or reverse phase liquid chromatography separation with time-of-flight detection and mass characterization (RP-UPLC/Mass spectrometry).

4. Methods for Making TF Antibodies

4.1. TF Antigen Preparation

The TF antigen used for isolation of the antibodies provided herein may be intact TF or a fragment of TF. The TF antigen may be, for example, in the form of an isolated protein or a protein expressed on the surface of a cell.

In some embodiments, the TF antigen is a non-naturally occurring variant of TF, such as a TF protein having an amino acid sequence or post-translational modification that does not occur in nature.

In some embodiments, the TF antigen is truncated by removal of, for example, intracellular or membrane-spanning sequences, or signal sequences. In some embodiments, the TF antigen is fused at its C-terminus to a human IgG1 Fc domain or a polyhistidine tag.

4.2. Methods of Making Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage-display libraries (see e.g., U.S. Pat. No. 8,258,082, which is incorporated by reference in its entirety) or, alternatively, using yeast-based libraries (see e.g., U.S. Pat. No. 8,691,730, which is incorporated by reference in its entirety).

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, CA, incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X₆₃-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

4.3. Methods of Making Chimeric Antibodies

Illustrative methods of making chimeric antibodies are described, for example, in U.S. Pat. No. 4,816,567; and Morrison et al., Proc. Natl. Acad Sci. USA, 1984, 81:6851-6855; each of which is incorporated by reference in its entirety. In some embodiments, a chimeric antibody is made by using recombinant techniques to combine a non-human variable region (e.g., a variable region derived from a mouse, rat, hamster, rabbit, or non-human primate, such as a monkey) with a human constant region.

4.4. Methods of Making Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad Sci. U.S.A., 1998, 95:8910-8915; Steinberger et al., J. Biol. Chem., 2000, 275:36073-36078; Queen et al., Proc. Natl. Acad Sci. U.S.A., 1989, 86:10029-10033; and U.S. Pat. Nos. 5,585,089, 5,693,761, 5,693,762, and 6,180,370; each of which is incorporated by reference in its entirety.

4.5. Methods of Making Human Antibodies

Human antibodies can be generated by a variety of techniques known in the art, for example by using transgenic animals (e.g., humanized mice). See, e.g., Jakobovits et al., Proc. Natl. Acad Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety). 4.6. Methods of Making Antibody Fragments

The antibody fragments provided herein may be made by any suitable method, including the illustrative methods described herein or those known in the art. Suitable methods include recombinant techniques and proteolytic digestion of whole antibodies.

Illustrative methods of making antibody fragments are described, for example, in Hudson et al., Nat. Med., 2003, 9:129-134, incorporated by reference in its entirety. Methods of making scFv antibodies are described, for example, in Plückthun, in The Pharmacology of Monoclonal Antibodies, vol. 113, Rosenburg and Moore eds., Springer-Verlag, New York, pp. 269-315 (1994); WO 93/16185; and U.S. Pat. Nos. 5,571,894 and 5,587,458; each of which is incorporated by reference in its entirety.

4.7. Methods of Making Alternative Scaffolds

The alternative scaffolds provided herein may be made by any suitable method, including the illustrative methods described herein or those known in the art. For example, methods of preparing Adnectins™ are described in Emanuel et al., mAbs, 2011, 3:38-48, incorporated by reference in its entirety. Methods of preparing iMabs are described in U.S. Pat. Pub. No. 2003/0215914, incorporated by reference in its entirety. Methods of preparing Anticalins® are described in Vogt and Skerra, Chem. Biochem., 2004, 5:191-199, incorporated by reference in its entirety. Methods of preparing Kunitz domains are described in Wagner et al., Biochem. & Biophys. Res. Comm., 1992, 186:118-1145, incorporated by reference in its entirety. Methods of preparing thioredoxin peptide aptamers are provided in Geyer and Brent, Meth. Enzymol., 2000, 328:171-208, incorporated by reference in its entirety. Methods of preparing Affibodies are provided in Fernandez, Curr. Opinion in Biotech., 2004, 15:364-373, incorporated by reference in its entirety. Methods of preparing DARPins are provided in Zahnd et al., J. Mol. Biol., 2007, 369:1015-1028, incorporated by reference in its entirety. Methods of preparing Affilins are provided in Ebersbach et al., J. Mol. Biol., 2007, 372:172-185, incorporated by reference in its entirety. Methods of preparing Tetranectins are provided in Graversen et al., J. Biol. Chem., 2000, 275:37390-37396, incorporated by reference in its entirety. Methods of preparing Avimers are provided in Silverman et al., Nature Biotech., 2005, 23:1556-1561, incorporated by reference in its entirety. Methods of preparing Fynomers are provided in Silacci et al., J. Biol. Chem., 2014, 289:14392-14398, incorporated by reference in its entirety.

Further information on alternative scaffolds is provided in Binz et al., Nat. Biotechnol., 2005 23:1257-1268; and Skerra, Current Opin. in Biotech., 2007 18:295-304, each of which is incorporated by reference in its entirety.

4.8. Methods of Making Multispecific Antibodies

The multispecific antibodies provided herein may be made by any suitable method, including the illustrative methods described herein or those known in the art. Methods of making common light chain antibodies are described in Merchant et al., Nature Biotechnol., 1998, 16:677-681, incorporated by reference in its entirety. Methods of making tetravalent bispecific antibodies are described in Coloma and Morrison, Nature Biotechnol., 1997, 15:159-163, incorporated by reference in its entirety. Methods of making hybrid immunoglobulins are described in Milstein and Cuello, Nature, 1983, 305:537-540; and Staerz and Bevan, Proc. Natl. Acad. Sci. USA, 1986, 83:1453-1457; each of which is incorporated by reference in its entirety. Methods of making immunoglobulins with knobs-into-holes modification are described in U.S. Pat. No. 5,731,168, incorporated by reference in its entirety. Methods of making immunoglobulins with electrostatic modifications are provided in WO 2009/089004, incorporated by reference in its entirety. Methods of making bispecific single chain antibodies are described in Traunecker et al., EMBO J., 1991, 10:3655-3659; and Gruber et al., J. Immunol., 1994, 152:5368-5374; each of which is incorporated by reference in its entirety. Methods of making single-chain antibodies, whose linker length may be varied, are described in U.S. Pat. Nos. 4,946,778 and 5,132,405, each of which is incorporated by reference in its entirety. Methods of making diabodies are described in Hollinger et al., Proc. Natl. Acad. Sci. USA, 1993, 90:6444-6448, incorporated by reference in its entirety. Methods of making triabodies and tetrabodies are described in Todorovska et al., J. Immunol. Methods, 2001, 248:47-66, incorporated by reference in its entirety. Methods of making trispecific F(ab′)₃ derivatives are described in Tutt et al. J. Immunol., 1991, 147:60-69, incorporated by reference in its entirety. Methods of making cross-linked antibodies are described in U.S. Pat. No. 4,676,980; Brennan et al., Science, 1985, 229:81-83; Staerz, et al. Nature, 1985, 314:628-631; and EP 0453082; each of which is incorporated by reference in its entirety. Methods of making antigen-binding domains assembled by leucine zippers are described in Kostelny et al., J. Immunol., 1992, 148:1547-1553, incorporated by reference in its entirety. Methods of making antibodies via the DNL approach are described in U.S. Pat. Nos. 7,521,056; 7,550,143; 7,534,866; and 7,527,787; each of which is incorporated by reference in its entirety. Methods of making hybrids of antibody and non-antibody molecules are described in WO 93/08829, incorporated by reference in its entirety, for examples of such antibodies. Methods of making DAF antibodies are described in U.S. Pat. Pub. No. 2008/0069820, incorporated by reference in its entirety. Methods of making antibodies via reduction and oxidation are described in Carlring et al., PLoS One, 2011, 6:e22533, incorporated by reference in its entirety. Methods of making DVD-Igs™ are described in U.S. Pat. No. 7,612,181, incorporated by reference in its entirety. Methods of making DARTs™ are described in Moore et al., Blood, 2011, 117:454-451, incorporated by reference in its entirety. Methods of making DuoBodies® are described in Labrijn et al., Proc. Natl. Acad. Sci. USA, 2013, 110:5145-5150; Gramer et al., mAbs, 2013, 5:962-972; and Labrijn et al., Nature Protocols, 2014, 9:2450-2463; each of which is incorporated by reference in its entirety. Methods of making antibodies comprising scFvs fused to the C-terminus of the CH3 from an IgG are described in Coloma and Morrison, Nature Biotechnol., 1997, 15:159-163, incorporated by reference in its entirety. Methods of making antibodies in which a Fab molecule is attached to the constant region of an immunoglobulin are described in Miler et al., J. Immunol., 2003, 170:4854-4861, incorporated by reference in its entirety. Methods of making CovX-Bodies are described in Doppalapudi et al., Proc. Natd. Acad. Sci. USA, 2010, 107:22611-22616, incorporated by reference in its entirety. Methods of making Fcab antibodies are described in Wozniak-Knopp et al., Protein Eng. Des. Sel., 2010, 23:289-297, incorporated by reference in its entirety. Methods of making TandAb® antibodies are described in Kipriyanov et al., J. Mol. Biol., 1999, 293:41-56 and Zhukovsky et al., Blood, 2013, 122:5116, each of which is incorporated by reference in its entirety. Methods of making tandem Fabs are described in WO 2015/103072, incorporated by reference in its entirety. Methods of making Zybodies' are described in LaFleur et al., mAbs, 2013, 5:208-218, incorporated by reference in its entirety.

4.9. Methods of Making Variants

In some embodiments, an antibody provided herein is an affinity matured variant of a parent antibody, which may be generated, for example, using phage display-based affinity maturation techniques. Briefly, one or more CDR residues may be mutated and the variant antibodies, or portions thereof, displayed on phage and screened for affinity. Such alterations may be made in CDR “hotspots,” or residues encoded by codons that undergo mutation at high frequency during the somatic maturation process (see Chowdhury, Methods Mol. Biol., 2008, 207:179-196, incorporated by reference in its entirety), and/or residues that contact the antigen.

Any suitable method can be used to introduce variability into a polynucleotide sequence(s) encoding an antibody, including error-prone PCR, chain shuffling, and oligonucleotide-directed mutagenesis such as trinucleotide-directed mutagenesis (TRIM). In some aspects, several CDR residues (e.g., 4-6 residues at a time) are randomized. CDR residues involved in antigen binding may be specifically identified, for example, using alanine scanning mutagenesis or modeling. CDR-H3 and CDR-L3 in particular are often targeted for mutation.

The introduction of diversity into the variable regions and/or CDRs can be used to produce a secondary library. The secondary library is then screened to identify antibody variants with improved affinity. Affinity maturation by constructing and reselecting from secondary libraries has been described, for example, in Hoogenboom et al., Methods in Molecular Biology, 2001, 178:1-37, incorporated by reference in its entirety.

4.10. Vectors, Host Cells, and Recombinant Methods

Also provided are isolated nucleic acids encoding TF antibodies, vectors comprising the nucleic acids, and host cells comprising the vectors and nucleic acids, as well as recombinant techniques for the production of the antibodies.

For recombinant production of an antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting, and any suitable host cell may be used to produce the antibodies provided herein.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. lichemformis), Pseudomonas (P. aeruginosa), and Streptomyces. One useful E. coli cloning host is E. coli 294, although other strains such as E. coli B, E. coli X₁₇₇₆, and E. coli W3110 are also suitable.

In addition to prokaryotes, eukaryotic microbes such as filamentous fungi or yeast are also suitable cloning or expression hosts for TF antibody-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells, baby hamster kidney (BHK) cells, Chinese hamster ovary (CHO), mouse sertoli cells, African green monkey kidney cells (VERO-76), and the like.

The host cells used to produce the TF antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469; or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio Technology, 1992, 10:163-167, incorporated by reference in its entirety) describes a procedure for isolating antibodies which are secreted to the periplasmic space of E. coli.

Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that comprise human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

5. Cytotoxic Agents

In some embodiments, ADCs provided herein comprise a cytotoxic agent. Cytotoxic agents may be considered for patients who have inflammatory diseases (e.g., autoimmune disorders). The cytotoxic agents provided herein include various immunosuppressive, anti-tumor or anti-cancer agents known in the art. In some embodiments, the cytotoxic agents cause destruction of cancer cells or immune cells.

Suitable cytotoxic agents include anti-angiogenic agents, pro-apoptotic agents, anti-mitotic agents, anti-kinase agents, alkylating agents, hormones, hormone agonists, hormone antagonists, chemokines, drugs, prodrugs, toxins, enzymes, antimetabolites, antibiotics, alkaloids, and radioactive isotopes.

In some embodiments, the cytotoxic agent comprises at least one of: calicheamycin, camptothecin, carboplatin, irinotecan, SN-38, carboplatin, camptothecan, cyclophosphamide, cytarabine, dacarbazine, docetaxel, dactinomycin, daunorubicin, doxorubicin, doxorubicin, etoposide, idarubicin, topotecan, vinca alkaloid, maytansinoid, maytansinoid analog, pyrrolobenzodiazepine, taxoid, duocarmycin, dolastatin, auristatin and derivatives thereof. In certain embodiments, the cytotoxic agent is monomethyl auristatin E (MMAE).

In some embodiments, the cytotoxic agent is a diagnostic agent, such as a radioactive isotope, a metal chelator, an enzyme, a fluorescent compound, a bioluminescent compound, or a chemiluminescent compound.

In some embodiments, the cytotoxic agent is a cytotoxic payload improved safety profile, for example XMT-1267 and other cytotoxic payloads described in Trail et al., Pharmacol Ther, 2018, 181:126-142.

6. Linkers

In some embodiments, ADCs provided herein comprise a linker. In some embodiments, an unbound linker comprises two reactive termini: an antibody conjugation reactive termini and an cytotoxic agent conjugation reactive termini. The antibody conjugation reactive terminus of the linker can be conjugated to the antibody through a cysteine thiol or lysine amine group on the antibody, typically a thiol-reactive group such as a double bond, a leaving group such as a chloro, bromo or iodo, an R-sulfanyl group or sulfonyl group, or an amine-reactive group such as a carboxyl group. The cytotoxic agent conjugation reactive terminus of the linker can be conjugated to the cytotoxic agent through formation of an amide bond with a basic amine or carboxyl group on the cytotoxin, typically a carboxyl or basic amine group.

In some embodiments, the linker is a non-cleavable linker. In some embodiments, the linker is a cleavable linker. In some embodiments, the cytotoxic agent is released from the ADC in a cell.

Suitable linkers of ADCs include labile linkers, acid labile linkers (e.g., hydrazone linkers), photolabile linkers, charged linkers, disulfide-containing linkers, peptidase-sensitive linkers (e.g., peptide linkers comprising amino acids, for example, valine and/or citrulline such as citrulline-valine or phenylalanine-lysine), p-glucuronide-linkers (See e.g., Graaf et al., Curr Pharm Des, 2002, 8:1391-1403), dimethyl linkers (See e.g., Chari et al., Cancer Research, 1992, 52:127-131; U.S. Pat. No. 5,208,020), thio-ether linkers, or hydrophilic linkers (See e.g., Kovtun et al., Cancer Res., 2010, 70:2528-2537). In certain embodiments, the cytotoxic agent is conjugated to the antibody using a valine-citrulline (vc) linker.

7. Methods for Making Antibody-Drug Conjugates

The antibody-drug conjugates (ADCs) provided herein can be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfoSIAB, sulfo-SMCC, and sulfo-SMPB, and SVSB (succinimidyl-(4-vinylsulfone)benzoate)). For example, a ricin immunotoxin can be prepared as described in Vitetta et al., Science, 1987, 238:1098. Additionally, the ADCs can be prepared using any suitable methods as disclosed in the art, e.g., in Bioconjugate Techniques, 2nd Ed., G. T. Hermanson, ed., Elsevier, San Francisco, 2008.

In some embodiments, the ADCs are made with site-specific conjugation techniques, resulting in homogeneous drug loading and avoiding ADC subpopulations with altered antigen-binding or pharmacokinetics. In some embodiments, “thiomabs” comprising cysteine substitutions at positions on the heavy and light chains are engineered to provide reactive thiol groups that do not disrupt immunoglobulin folding and assembly or alter antigen binding (Junutula et al., J. Immunol. Meth., 2008, 332: 41-52; Junutula et al., Nat. Biotechnol., 2008, 26: 925-932,). In some embodiments, selenocysteine is co-translationally inserted into an antibody sequence by recoding the stop codon UGA from termination to selenocysteine insertion, allowing site specific covalent conjugation at the nucleophilic selenol group of selenocysteine in the presence of the other natural amino acids (See e.g., Hofer et al., Proc. Natl. Acad. Sci. USA, 2008, 105:12451-12456; Hofer et al., Biochemistry, 2009, 48(50):12047-12057). In certain embodiments, ADCs were synthesized as described in Behrens et al., Mol Pharm, 2015, 12:3986-98.

8. Assays

A variety of assays known in the art may be used to identify and characterize anti-TF antibodies and anti-TF ADCs provided herein.

8.1. Binding, Competition, and Epitope Mapping Assays

Specific antigen-binding activity of the antibodies provided herein may be evaluated by any suitable method, including using SPR, BLI, RIA and MSD-SET, as described elsewhere in this disclosure. Additionally, antigen-binding activity may be evaluated by ELISA assays and Western blot assays.

Assays for measuring competition between two antibodies, or an antibody and another molecule (e.g., one or more ligands of TF) are described elsewhere in this disclosure and, for example, in Harlow and Lane, Antibodies: A Laboratory Manual ch.14, 1988, Cold Spring Harbor Laboratory, Cold Spring Harbor, N.Y, incorporated by reference in its entirety.

Assays for mapping the epitopes to which the antibodies provided herein bind are described, for example, in Morris “Epitope Mapping Protocols,” in Methods in Molecular Biology vol. 66, 1996, Humana Press, Totowa, N.J., incorporated by reference in its entirety.

In some embodiments, the epitope is determined by peptide competition. In some embodiments, the epitope is determined by mass spectrometry. In some embodiments, the epitope is determined by crystallography.

8.2. Thrombin Generation, FXa Conversion, and TF Signaling Assays

Thrombin generation in the presence of the antibodies provided herein can be determined by the Thrombin Generation Assay (TGA), as described elsewhere in this disclosure.

Assays for measuring FXa conversion in the presence of the antibodies provided herein are described elsewhere in this disclosure.

Inhibition of TF signaling can be determined by measuring the production of a cytokine regulated by the TF signaling, such as IL8 and GM-CSF. Assays for determining the IL8 and/or GM-CSF level are provided elsewhere in this disclosure and, for example, in Hjortoe et al., Blood, 2004, 103:3029-3037.

8.3. Assays for Effector Functions

Effector function following treatment with the antibodies provided herein may be evaluated using a variety of in vitro and in vivo assays known in the art, including those described in Ravetch and Kinet, Annu. Rev. Immunol., 1991, 9:457-492; U.S. Pat. Nos. 5,500,362, 5,821,337; Hellstrom et al., Proc. Nat'l Acad. Sci. USA, 1986, 83:7059-7063; Hellstrom et al., Proc. Nat'l Acad. Sci. USA, 1985, 82:1499-1502; Bruggemann et al., J. Exp. Med., 1987, 166:1351-1361; Clynes et al., Proc. Nat'l Acad. Sci. USA, 1998, 95:652-656; WO 2006/029879; WO 2005/100402; Gazzano-Santoro et al., J. Immunol. Methods, 1996, 202:163-171; Cragg et al., Blood, 2003, 101:1045-1052; Cragg et al. Blood, 2004, 103:2738-2743; and Petkova et al., Int'l. Immunol., 2006, 18:1759-1769; each of which is incorporated by reference in its entirety.

8.4. Cytotoxicity Assays and In Vivo Studies

Assays for evaluating cytotoxicity of the antibody-drug conjugates (ADCs) provided herein are described elsewhere in this disclosure.

Xenograft studies in immune compromised mice for evaluating the in vivo efficacy of the ADCs provided herein are described elsewhere in this disclosure.

Syngeneic studies in immune competent mice for evaluating the in vivo efficacy of the ADCs are included in this disclosure.

8.5. Immunohistochemistry (IHC) Assays

Immunohistochemistry (IHC) assays for evaluating the TF expression in patient samples are described elsewhere in this disclosure.

8.6. Chimeric Construct Mapping and Epitope Binning Assays

Epitope binding differences between the anti-human TF antibodies provided herein can be determined by the chimeric TF construct mapping experiments and the epitope binning assays, as described elsewhere in this disclosure.

9. Pharmaceutical Compositions

The antibodies provided herein can be formulated in any appropriate pharmaceutical composition and administered by any suitable route of administration. The route of administration of the pharmaceutical composition can be according to known methods, e.g. orally, through injection by intravenous, intraperitoneal, intracerebral (intra-parenchymal), intracerebroventricular, intramuscular, intra-ocular, intraarterial, intraportal, intralesional routes, intramedullary, intrathecal, intraventricular, transdermal, subcutaneous, intraperitoneal, intranasal, enteral, topical, sublingual, urethral, vaginal, or rectal means, by sustained release systems or by implantation devices. Where desired, the compositions may be administered by bolus injection or continuously by infusion, or by implantation device. In certain embodiments, suitable routes of administration include, but are not limited to, the intraarterial, intradermal, intramuscular, intraperitoneal, intravenous, nasal, parenteral, topical, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

9.1. Parenteral Dosage Forms

In certain embodiments, the antibodies provided herein are formulated as parenteral dosage forms. Parenteral dosage forms can be administered to subjects by various routes including, but not limited to, subcutaneous, intravenous (including infusions and bolus injections), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry (e.g., lyophilized) products ready to be dissolved or suspended in a pharmaceutically acceptable vehicle for injection, suspensions ready for injection, and emulsions.

10. Dosage and Unit Dosage Forms

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies or ADCs.

The amount of the antibody/ADC or composition which will be effective in the prevention or treatment of a disorder or one or more symptoms thereof can vary with the nature and severity of the disease or condition, and the route by which the antibody/ADC is administered. The frequency and dosage can also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies or ADCs provided herein are also encompassed by the dosage amounts and dose frequency schedules provided herein. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

As discussed in more detail elsewhere in this disclosure, an antibody or ADC provided herein may optionally be administered with one or more additional agents useful to prevent or treat a disease or disorder. The effective amount of such additional agents may depend on the amount of ADC present in the formulation, the type of disorder or treatment, and the other factors known in the art or described herein.

11. Therapeutic Applications

For therapeutic applications, the antibodies of the invention are administered to a subject, generally a mammal, generally a human, in a pharmaceutically acceptable dosage form such as those known in the art and those discussed above. For example, the antibodies of the invention may be administered to a subject intravenously as a bolus or by continuous infusion over a period of time, by intravitreal, intraperitoneal, intra-cerebrospinal, subcutaneous, intra-articular, intrasynovial, intrathecal, intratumoral, or topical routes. In certain embodiments, administration is via intravenous, intramuscular, intratumoral, subcutaneous, intrasynovial, intraocular, intraplaque, or intradermal injection of the antibody or of an expression vector having cDNA encoding the antibody. The vector can be a replication-deficient adenoviral vector, retroviral vector or other viral vectors carrying a cDNA encoding the antibody.

In some embodiments, the patient is treated with an effective amount of one or more replication-deficient adenoviral vectors, or one or more adeno-associated vectors carrying cDNA encoding the antibody.

The antibodies provided herein may be useful for the treatment of inflammatory diseases involving TF. As used the term “inflammatory disease” refers broadly to any disease, disorder, injury or condition characterized by inflammation (local or systemic, acute or chronic). As used, “inflammatory disease” also encompasses autoimmune diseases. Further, as used, the term “inflammatory diseases” also encompass symptoms of inflammation.

Examples of symptoms of inflammation include, without limitation, increased concentration or expression of inflammatory cytokines and chemokines (local or systemic), swelling, pain, fibrosis, increased erythrocyte sedimentation rate (ESR), infiltration of mononuclear cells and/or granulocytes at the diseased or injured site (e.g., interstitial fluid of lungs, alveoli, site of acute injury, etc.), enlarged spleen, weight loss, hypoxemia as determined using pulse oximetry (indicative of an inflammatory disease affecting the respiratory system), reduced alveolar fluid clearance, change in stool consistency (e.g., softening of the subject's stool), diarrhea (e.g., chronic diarrhea), hematochezia, occult blood, rubor (redness) at the site of inflammation or injury, calor (increased heat) at the site of inflammation or injury, functio laesa (loss of function) at the site of inflammation or injury or in the disease organ, rash, headache, fever, nausea, or local tissue or cell damage.

Treatment of an inflammatory disease using the methods of the present disclosure results in reducing or ameliorating one or more adverse symptoms of the inflammatory disease or other effects associated with the contraction or progression of the inflammatory disease.

In some instances, an increase in total leukocyte count is a symptom of an inflammatory disease (e.g., colitis, inflammatory bowel disease, arthritis, acute lung injury, acute respiratory distress syndrome (ARDS), and Respiratory Syncytial Virus (RSV)). In certain embodiments, upon administration of an antibody or ADC provided herein, the antibody or ADC reduces the total leukocyte count by, for example, at least 5%, 10%, 15%, 20%, 25%, 30%, 35%, 40%, 45%, 50%, 55%, 60%, 65%, 70%, 75%, 80%, 85%, 90%, or 95% relative to baseline levels and/or another anti-inflammatory agent. Methods for measuring total leukocyte count are known in the art. In certain embodiments, the total leukocyte count is determined using light microscopy.

In some instances, an increase in total granulocyte count (e.g. total neutrophil count, total eosinophil count, total basophil count) is a symptom of inflammatory disease (e.g., colitis, inflammatory bowel disease, arthritis, acute lung injury, acute respiratory distress syndrome (ARDS), and Respiratory Syncytial Virus (RSV)). In certain embodiments, upon administration of an antibody or ADC provided herein, the antibody or ADC reduces the total granulocyte count by, for example at least 5%, 10%, 15%, 20%, 25%, 30%, 35%, 40%, 45%, 50%, 55%, 60%, 65%, 70%, 75%, 80%, 85%, 90%, or 95% relative to baseline levels and/or another anti-inflammatory agent. Methods for measuring total granulocyte count are known in the art. In certain embodiments, the total granulocyte count is determined using immunohistochemical (IHC) analysis on a tissue sample or serum sample. In certain embodiments, the total granulocyte count is determined using bronchoalveolar lavage (BAL) fluid differential cell counts. Methods for conducting BAL fluid differential cell counts and analysis are known in the art (see, for example, Choi S H, et al. PLoS One. 2014; 9(5):e97346, which is incorporated by reference in its entirety).

In some instances, an increase in total mononuclear cell count (e.g. total macrophage count, total lymphocyte count) is a symptom of inflammatory disease (e.g., colitis, inflammatory bowel disease, arthritis, acute lung injury, acute respiratory distress syndrome (ARDS), and Respiratory Syncytial Virus (RSV)). In certain embodiments, upon administration of an antibody or ADC provided herein, the antibody or ADC reduces the total mononuclear cell count by, for example at least 5%, 10%, 15%, 20%, 25%, 30%, 35%, 40%, 45%, 50%, 55%, 60%, 65%, 70%, 75%, 80%, 85%, 90%, or 95% relative to baseline levels and/or another anti-inflammatory agent. Methods for measuring total mononuclear cell count are known in the art. In certain embodiments, the total mononuclear cell count is determined using immunohistochemical (IHC) analysis on a tissue sample or serum sample. In certain embodiments, the total mononuclear cell count is determined using bronchoalveolar lavage (BAL) fluid differential cell counts. Methods for conducting BAL fluid differential cell counts and analysis are known in the art (see, for example, Choi S H, et al. PLoS One. 2014; 9(5):e97346, which is incorporated by reference in its entirety).

In certain embodiments, treatment with an antibody or ADC of the present disclosure results in a decrease in M1 macrophages and/or a decrease in M2 macrophages. In certain embodiments, treatment with an antibody or ADC of the present disclosure results in a decrease in M1 macrophages and/or an increase in M2 macrophages. In certain inflammatory diseases, elevated M2 macrophages have been associated with the asymptomatic state of the disease or disease regression. (See Hu, Kebin, et al., Journal of Immunology Research, 2018, which is incorporated by reference in its entirety).

In some instances, splenomegaly (enlarged spleen) is a symptom of inflammatory disease. In certain embodiments, upon administration of an antibody or ADC provided herein, the antibody or ADC reduces the weight of the spleen, reduces the size of the spleen, or eliminates/reverses splenomegaly relative to baseline levels or relative to a different anti-inflammatory agent. In a clinical setting, measuring weight of the spleen may not be practical. In such cases, the progression (or reversal) of splenomegaly can be measured using methods known in the art (e.g., palpation, percussion, ultrasound, computerized tomography (CT) scan or magnetic resonance imagining (MRI)). Ultrasound, computerized tomography (CT) scan and magnetic resonance imagining (MRI) allow for visualization of the spleen. Ultrasound or computerized tomography (CT) scan help determine the size of your spleen and determine whether it's crowding other organs. Magnetic resonance imagining (MRI) allows the clinician to trace blood flow through the spleen.

In some instances, fibrosis (e.g., fibrosis of the lung tissue or fibrosis at the site of inflammation) is a symptom of inflammatory disease. Fibrosis is often characteristic of chronic inflammation. In certain embodiments, upon administration of an antibody or ADC provided herein, the antibody or ADC reduces fibrosis (e.g. fibrosis in the lungs, skin or liver) relative to baseline levels or relative to a different anti-inflammatory agent. Changes in fibrosis can be measured using IHC analysis of the tissue or by Quantitative High Resolution Computed Tomography (qHRCT).

In some instances, increased erythrocyte sedimentation rate (ESR) is an indicator of an inflammatory disease. The ESR is the rate at which red blood cells in anticoagulated whole blood descend in a standardized tube over a period of one hour. It is a common hematology test, and is a non-specific measure of inflammation. In certain embodiments, upon administration of an antibody or ADC provided herein, the antibody or ADC reduces the ESR by, for example at least 5%, 10%, 15%, 20%, 25%, 30%, 35%, 40%, 45%, 50%, 55%, 60%, 65%, 70%, 75%, 80%, 85%, 90%, or 95%, relative to baseline levels and/or another anti-inflammatory agent.

In some instances, changes in stool consistency, softening of the stool, and/or diarrhea are symptom(s) of an inflammatory disease (e.g., colitis, inflammatory bowel disease (IBD)). For example a subject with an inflammatory disease may present with loose stool that is classified as greater than 4, 5, or 6 on the Bristol Stool Chart. The Bristol Stool Form Scale (BSFS) or Bristol Stool Chart was developed as a method of assessing intestinal transit time in adults (see Lewis S J, et al., Scand J Gastrotrnterol, 32:920-924 (1997), which is incorporated by reference in its entirety). It is a paper chart scale composed of 2-dimensional representations of the various stool types ordered in a vertical fashion with each stool type depicted in association with a text description of each stool type. The BSFS is widely used in patients with functional gastrointestinal disorders (FGIDs) in clinical care. An example of methods and devices for measuring stool consistency is provided in U.S. application Ser. No. 13/592,906, incorporated by reference in its entirety. In cases where a subject having an inflammatory disease (e.g. colitis, IBD) presents with loose stool, upon administration of an antibody or ADC provided herein, the antibody or ADC results in hardening of the stool relative to baseline levels and/or a different anti-inflammatory agent. In certain embodiments, upon administration of an antibody or ADC provided herein, the antibody or ADC results in a stool consistency classified as 3 on the BSFS. Other endpoints or symptoms that may be improved by treatment with an antibody or ADC provided herein include hematochezia, stool frequency, fecal urgency and severity, and abdominal pain.

In some instances, hematochezia and/or occult blood is a symptom of an inflammatory disease (e.g., colitis or IBD). Hematochezia is the passage of blood from the anus (typically in or with stool). Hematochezia can be determined by visual examination of the stool. In contrast, occult blood is blood in the stool that is not visibly apparent, and may also be indicative of an inflammatory disease. A more accurate method to determine changes in the amount of blood in stool (particularly occult blood) is by using a hemoccult test, fecal occult blood test, or immunochemical hemagglutination test. Methods for conducting a hemoccult test are known in the art (for example, the test can be performed using Hemoccult slide kit, SmithKline Diagnostics, Inc. and manufacturer instructions). Methods for conducting immunochemical hemagglutination tests are also known in the art and utilize an antibody specific for human hemoglobin for detection.

In some instances, a reduction in the net alveolar fluid clearance (AFC) or AFC impairment is a symptom of the inflammatory disease (e.g., acute respiratory distress syndrome (ARDS) and acute lung injury). In certain embodiments, upon administration of an antibody or ADC provided herein, the antibody or ADC increases the AFC relative to baseline levels or a different anti-inflammatory agent. AFC can be measured using methods known in the art, for example, measurement of sequential edema fluid protein concentrations. Methods for determining changes in AFC using measurement of sequential edema fluid protein concentrations are provided, for example, in Ware, L. B. and Michael, M. A., American journal of respiratory and critical care medicine, 163.6 (2001): 1376-1383, which is incorporated by reference in its entirety.

Inflammation can directly or indirectly cause cell, tissue or organ damage to multiple cells, tissues or organs, or to a single cell type, tissue type or organ. Exemplary tissues and organs that may show damage depend on the inflammatory disease and include epithelial or mucosal tissue, gastrointestinal tract, intestine, pancreas, thymus, liver, kidney, spleen, skin, or skeletal joint (e.g., knee, ankle, hip, shoulder, wrist, finger, toe, or elbow). Treatment according to the present disclosure may result in a reduction or inhibition of tissue damage, or may result in regeneration of damaged organs or tissues (e.g., skin, mucosa, liver, lungs, etc.).

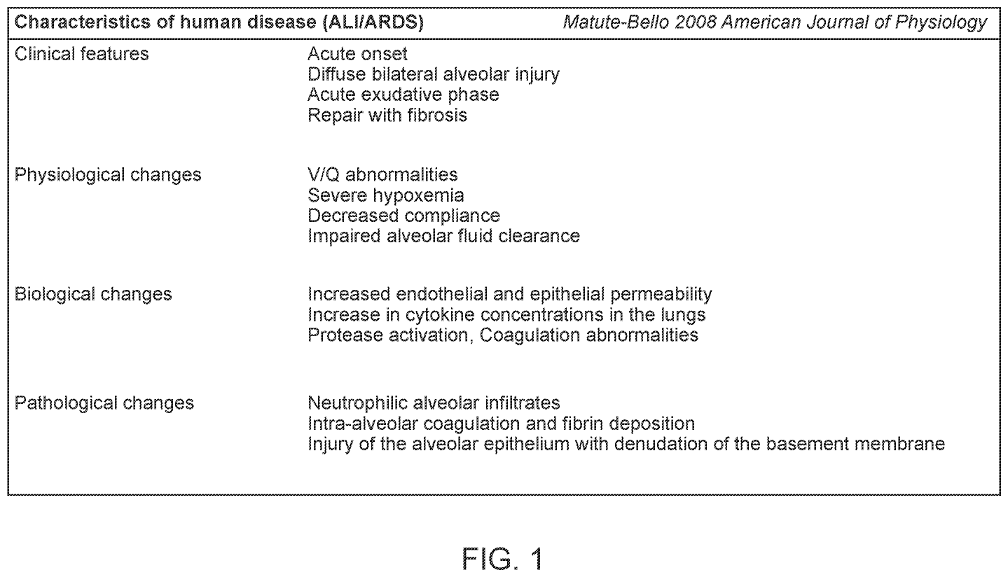

FIG. 1 provides examples of the characteristics/symptoms of ALI and ARDS in humans. (See Matute-Bello 2008 American Journal of Physiology, which is incorporated by reference in its entirety).

In some embodiments, provided herein is a method of delaying the onset of an inflammatory disease in a subject in need thereof by administering an effective amount of an antibody or ADC provided herein to the subject.

In some embodiments, provided herein is a method of preventing the onset of an inflammatory disease in a subject in need thereof by administering an effective amount of an antibody or ADC provided herein to the subject.

In some embodiments, provided herein is a method for extending the period of overall survival, median survival time, or progression-free survival in a subject in need thereof by administering an effective amount of an antibody or ADC provided herein to the subject.

In some embodiments, provided herein is a method for treating a subject who has become resistant to a standard of care therapeutic by administering an effective amount of an antibody or ADC provided herein to the subject.

In some embodiments, the disease or condition that can benefit from treatment with an anti-TF antibody is a disease or condition involving inflammation. In certain embodiments, the inflammatory disease is colitis, inflammatory bowel disease, arthritis, acute lung injury, acute respiratory distress syndrome (ARDS), or Respiratory Syncytial Virus (RSV). In some embodiments, the disease or condition that can benefit from treatment with an anti-TF antibody is a disease or condition involving vascular inflammation.

In some embodiments, the anti-TF antibodies or ADCs provided herein are provided for use as a medicament for the treatment of a disease or condition involving inflammation. In some embodiments, the anti-TF antibodies provided herein are provided for use in the manufacture or preparation of a medicament for the treatment of an inflammatory disease. In certain embodiments, the inflammatory disease is colitis, inflammatory bowel disease, arthritis, acute lung injury, acute respiratory distress syndrome (ARDS), or Respiratory Syncytial Virus (RSV). In some embodiments, the anti-TF antibodies or ADCs provided herein are provided for use as a medicament for the treatment of a disease or condition involving vascular inflammation. In some embodiments, the anti-TF antibodies provided herein are provided for use in the manufacture or preparation of a medicament for the treatment of a disease or condition involving vascular inflammation.

In some embodiments, provided herein is a method of treating an inflammatory disease in a subject in need thereof by administering an effective amount of an anti-TF antibody provided herein to the subject. In certain embodiments, the inflammatory disease is colitis, inflammatory bowel disease, arthritis, acute lung injury, acute respiratory distress syndrome (ARDS), or Respiratory Syncytial Virus (RSV). In some embodiments, provided herein is a method of treating a disease or condition involving vascular inflammation in a subject in need thereof by administering an effective amount of an anti-TF antibody or ADC provided herein to the subject.

In some embodiments, provided herein is a method of delaying the onset of an inflammatory disease in a subject in need thereof by administering an effective amount of an antibody provided herein to the subject.

In some embodiments, provided herein is a method of preventing the onset of an inflammatory disease in a subject in need thereof by administering an effective amount of an antibody provided herein to the subject.

In some embodiments, provided herein is a method of delaying the onset of a disease or condition involving vascular inflammation in a subject in need thereof by administering an effective amount of an antibody provided herein to the subject.

In some embodiments, provided herein is a method of preventing the onset of a disease or condition involving vascular inflammation in a subject in need thereof by administering an effective amount of an antibody provided herein to the subject.

12. Inflammation and Inflammatory Diseases

Inflammation can be classified as either acute or chronic. Acute inflammation is the body's initial response to harmful stimuli and is achieved by increased movement of plasma and white blood cells (e.g., leukocytes, e.g., mononuclear cells and granulocytes) from the blood to the damaged tissue. That initiates a cascade of biochemical events that result in a mature inflammatory response, including various cells in the local vasculature, immune system, and damaged tissue. In contrast, chronic inflammation, results in a progressive shift of the cell types present at the site of inflammation and is characterized by the simultaneous destruction and healing of tissue from the inflammatory process. Chronic inflammation can also lead to host diseases including, but not limited to, hay fever, periodontitis, atherosclerosis, rheumatoid arthritis, and cancer, highlighting the need for the body to closely regulated by the body.

Examples of inflammatory diseases that are contemplated in the methods of this disclosure include: colitis, inflammatory bowel disease, arthritis, acute lung injury (ALI), acute respiratory distress syndrome (ARDS), and Respiratory Syncytial Virus (RSV).

Non-limiting examples of inflammatory diseases include, but are not limited to, acne vulgaris, acute lung injury, acute respiratory distress syndrome, asthma, autoimmune diseases (e.g., acute disseminated encephalomyelitis (ADEM)), Addison's disease, agammaglbulinemia, alopecia areata, amyotrophic lateral sclerosis, ankylosing spondylitis, antiphospholipid syndrome, antisynthetase syndrome, atopic allergy, atopic dermatitis, autoimmune aplastic anemia, autoimmune cardiomyopathy, autoimmune enteropathy, autoimmunehemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo concentric sclerosis, Behcet's disease, Berger's disease, Bickerstaff s encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, Churg-Strauss syndrome, cicatricial pemphigoid, Cogan syndrome, colitis, cold agglutinin disease, complement component 2 deficiency, contact dermatitis, cranial arteritis, CREST syndrome, Crohn's disease, Cushing's syndrome, cutaneous leukocytoclastic vasculitis, Dego's disease, Dercum's disease, dermatitis herpetiformis, dermatomyositis, diabetes mellitus type 1, diffuse cutaneous systemic sclerosis, Dressler's syndrome, drug-induced lupus, discoid lupus erythematosus, eczema, endometriosis, enthesitis-related arthritis, eosinophilic fasciitis, eosinophilic gastroenteritis, epidermolysis bullosa acquisita, erythema nodosum, erythroblastosis fetalis, essential mixed cryoglobulinemia, Evan's syndrome, fibrodysplasia ossificans progressive, fibrosing alveolitis, gastritis, gastrointestinal pemphigoid, giant cell arteritis, glomerulonephritis, Goodpasture's syndrome, Grave's disease, Guillain-Barre syndrome, Hashimoto's encephalopathy, Hashimoto's thyroiditis, Henoch-Schonlein purpura, herpes gestationis, hidradenitis suppurativa, Hughes-Stovin syndrome, hypogammaglobulinemia, idiopathic inflammatory demyelinating diseases, idiopathic pulmonary fibrosis, idiopathic thrombocytopenic purpura, IgA nephropathy, inclusion body myositis, chronic inflammatory demyelinating polyneuropathy, interstitial cystitis, juvenile idiopathic arthritis, Kawasaki's disease, Lambert-Eaton myasthenic syndrome, leukocytoclastic vasculitis, lichen planus, lichen sclerosus, linear IgA disease, lupus erythematosus, Majeed syndrome, Meniere's disease, microscopic polyangiitis, mixed connective tissue disease, morphea, Mucha-Habermann disease, myasthenia gravis, myositis, narcolepsy, neuromyelitis optica, neuromyotonia, ocular cicatricial pemphigoid, opsoclonus myoclonus syndrome, Ord's thyroiditis, palindromic rheumatism, PANDAS, paraneoplastic cerebellar degeneration, paroxysmal nocturnal hemoglobinuria, Parry Romberg syndrome, Parsonage-Turner syndrome, pars planitis, pemphigus vulgaris, pernicious anemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriatic arthritis, pyoderma gangrenosum, pure red cell aplasia, Rasmussen's encephalitis, raynaud phenomenon, relapsing polychondritis, Reiter's syndrome, respiratory syncytial virus (RSV), restless leg syndrome, retroperitoneal fibrosis, rheumatic fever, Schnitzler syndrome, scleritis, scleroderma, serum sickness, Sjogren's syndrome, spondyloarthropathy, stiff person syndrome, subacute bacterial endocarditis, Susac's syndrome, Sweet's syndrome, sympathetic ophthalmia, Takayasu's arteritis, temporal arteritis, thrombocytopenia, Tolosa-Hunt syndrome, transverse myelitis, ulcerative colitis, undifferentiated connective tissue disease, undifferentiated spondyloarthropathy, vitiligo, and Wegener's granulomatosis), celiac disease, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, interstitial cystitis, and osteoarthritis.

In some embodiments, the term “inflammatory diseases” includes viral infections. In some embodiments, inflammatory disease includes severe acute respiratory syndrome coronavirus 2 (SARS-CoV-2). In some embodiments, the anti-TF antibody as described herein is used to treat a pathogenic virus, such as respiratory syncytial virus (RSV), poliovirus, herpes simplex virus, hepatitis A virus, rotavirus, adenovirus, SARS-CoV-2 and influenza type A virus. In some embodiments, the pathogenic virus is selected from: Herpesviridae, Poxviridae, Hepadnaviridae, Coronaviridae, Flaviviridae, Togaviridae, Retroviridae, Orthomyxoviridae, Arenaviridae, Bunyaviridae, Filoviridae, Paramyxoviridae, and Rhabdoviridae. In one embodiment, said virus is selected from the group consisting of Herpes simplex, type 1, Herpes simplex, type 2, Varicella-zoster virus, Epstein-Barr virus, Human cytomegalovirus, Human herpesvirus, Smallpox, Hepatitis B virus, Severe acute respiratory syndrome virus, Hepatitis C virus, yellow fever virus, dengue virus, West Nile virus, TBE virus, Zika virus, Rubella virus, Human immunodeficiency virus (HIV), Influenza virus, Lassa virus, Crimean-Congo, hemorrhagic fever virus, Hantaan virus, Ebola virus, Marburg virus, Measles virus, Mumps virus, Parainfluenza virus, Respiratory syncytial virus, Rabies virus, and Hepatitis D virus (HDV).

Several autoimmune diseases are considered inflammatory diseases and/or cause inflammation through a variety of mechanisms. Treatment of autoimmune disease using an antibody or ADC provided herein is also contemplated in the present disclosure. Non-limiting examples of inflammatory diseases include: Examples of autoimmune diseases or disorders include arthritis such as rheumatoid arthritis, acute arthritis, rheumatoid arthritis, gouty arthritis, acute gouty arthritis, acute immunological arthritis, chronic inflammatory arthritis, osteoarthritis, type-II collagen evoked arthritis, infectious arthritis, Lyme arthritis, proliferative arthritis, psoriatic arthritis, Still's disease, spondyloarthritis, juvenile-onset rheumatoid arthritis, osteoarthritis, arthritis chronica progrediente, osteoarthritis, chronic primary multiple polyarthritis chronica primaria, reactive arthritis and ankylosing spondylitis; inflammatory hyperproliferative skin disease; psoriasis such as psoriasis vulgaris, gutatte psoriasis, pustular psoriasis, nail psoriasis; atopy, (e.g., atopic diseases, e.g., hay fever and Job syndrome); dermatitis (e.g., contact dermatitis, chronic contact dermatitis, erythroderma, allergic dermatitis, allergic contact dermatitis, herpetic dermatitis, monetary dermatitis, seborrheic dermatitis, non-specific dermatitis, primary irritant contact dermatitis, and atopic dermatitis); x-linked hyper IgM syndrome; allergic intraocular inflammatory disease; urticaria, e.g. chronic allergic urticaria, Chronic idiopathic urticaria and chronic autoimmune urticaria; myositis; polymyositis/dermatomyositis; juvenile dermatomyositis; toxic epidermal necrosis; scleroderma, e.g. systemic scleroderma; sclerosis, e.g. whole body Sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic scler optic neuromyelitis (NMO); inflammatory bowel disease (IBD), e.g. Crohn's disease, autoimmune-mediated gastrointestinal disease, colitis, ulcerative colitis, ulcerative colitis, microscopic colitis, collagen formation Colitis, colitis polyposa, necrotizing enterocolitis, full thickness colitis, and autoimmune inflammatory bowel disease; enteritis; gangrenous scleroderma; nodular erythema; primary sclerosing cholangitis Dyspnea syndrome, e.g. adult or acute dyspnea syndrome (ARDS); meningitis; inflammation of all or part of the uvea; iritis; choroiditis; autoimmune blood disease; rheumatic spondylitis; Synovitis; hereditary angioedema; cranial nerve disorders such as meningitis; gestational herpes; gestational pemphigoid; pruritis scroti; autoimmune ovarian dysfunction; autoimmune symptoms Sudden hearing loss due to IgE-mediated diseases such as Anaphyki Encephalitis, e.g. ramssen encephalopathy and limbic and/or brainstem encephalitis; uveitis, e.g. anterior uveitis, acute anterior uveitis, granulomatous uveitis, Non-granulomatous uveitis, lens antigenic uveitis, posterior uveitis, or autoimmune uveitis; glomerulonephritis (GN) with or without nephrotic syndrome, e.g. chronic or acute thread Globe nephritis, primary GN, immune-mediated GN, membranous GN (membranous nephropathy), idiopathic membranous GN or idiopathic membranous nephropathy, membranous or membranous proliferative GN (MPGN), e.g. type I And type II, and rapidly progressive GN; proliferative nephritis; autoimmune multiple endocrine insufficiency; balanitis, e.g. plasma cell localized bullitis; glans foreskinitis; efferent annular erythema; Erythema multiforme; granulomas of the ring; gloss lichen; Atrophic lichen; bidar lichen; spiny lichen; lichen planus; lamellar ichthyosis; exfoliative keratosis; precancerous keratosis; gangrenous scleroderma; allergic symptoms and responses; Reactions; eczema such as allergic and atopic eczema, sebum-deficient eczema, vesicular eczema, and vesicular palmoplantar eczema; asthma such as bronchial asthma, bronchial asthma, and autoimmune asthma; T cell wetting and symptoms including chronic inflammatory response; immune response to foreign antigens such as fetal ABO blood group during pregnancy; chronic lung inflammatory disease; autoimmune myocarditis; leukocyte adhesion deficiency; lupus such as lupus nephritis; Lupus encephalitis, childhood lupus, non-renal lupus, extra-renal lupus, discoid lupus and discoid lupus erythematosus, alopecia lupus, systemic lupus Temato SLE, cutaneous SLE, subacute cutaneous SLE, neonatal lupus syndrome (NLE), and disseminated lupus, lupus erythematosus (lupus erythematosus disseminatus); juvenile onset (type I) diabetes, e.g. pediatric insulin dependence Diabetes mellitus (IDDM), adult-onset diabetes (type II diabetes), autoimmune diabetes, idiopathic diabetes insipidus, diabetic retinopathy, diabetic nephropathy, and diabetic aortic disease; mediated by cytokines and T lymphocytes Immune response associated with acute and delayed hypersensitivity; tuberculosis; sarcoidosis; granulomatosis, e.g. lymphoma-like granulomatosis; Wegener's granulomatosis; agranulocytosis; vasculitides, e.g. vasculitis, Macrovascular vasculitis, rheumatoid polymyalgia and giant cell (Takayasu) arteritis, medium vascular vasculitis, Kawasaki disease, nodular polyarteritis/nodal periarteritis, microscopic polyangiitis, Immune vasculitis, CNS vasculitis, cutaneous vasculitis, hypersensitivity vasculitis, necrotizing vasculitis, systemic necrotizing vasculitis, ANCA-related vasculitis, Churg-Strauss vasculitis or syndrome (CSS), And ANCA-related small vessel vasculitis; temporal arteritis; aplastic anemia; autoimmune aplastic anemia; Coombs positive anemia; Diamond Blackfan anemia; hemolytic anemia or immune hemolytic anemia (e.g., autoimmunity hemolytic anemia (AIHA)), perniciosemia (anemia perniciosa); Addison disease; true red cell anemia or red blood cell aplasia (PRCA); factor VIII deficiency; hemophilia A, autoimmune neutropenia; pancytopenia; leukopenia; diseases including leukocyte leakage; CNS inflammatory disease; multi-organ injury syndrome, e.g. secondary to sepsis, trauma or bleeding; antigen-antibody Complex-mediated disease; glomerular basement membrane antibody disease; antiphospholipid antibody syndrome; allergic god Behcet's disease/syndrome; Castleman syndrome; Goodpasture syndrome; Reynaud syndrome; Sjogren syndrome; Stevens-Johnson syndrome; Bullous pemphigoid and cutaneous pemphigoid, pemphigus, pemphigus vulgaris, deciduous pemphigus, pemphigus mucus-membrane pemphigoid, and erythematous pemphigus; autoimmune multi-endocrine endocrinopathy Reiter's disease or syndrome; heat injury; pre-eclampsia; immune complex disorders such as immune complex nephritis and antibody-mediated nephritis; multiple neuropathy; chronic nephropathy such as IgM multiple neuropathy and IgM-mediated neurosis; thrombocytopenia (e.g., in patients with myocardial infarction), e.g. thrombotic thrombocytopenic purpura (TTP), post-transfusion purpura (PTP), heparin-induced thrombocytopenia, autoimmune or immune-mediated thrombocytopenia, idiopathic thrombocytopenic purpura (ITP), and chronic or acute ITP; scleritis, e.g. idiopathic corneal scleritis, and episclerosis: testis and ovary Autoimmune diseases such as autoimmune orchitis; primary hypothyroidism; hypoparathyroidism; autoimmune endocrine diseases such as thyroiditis, autoimmune thyroiditis, Hashimoto's disease, chronic thyroiditis (Hashimoto's thyroiditis), or subacute thyroiditis, autoimmune thyroid disease, idiopathic hypothyroidism, Grave's disease, multigland syndrome, autoimmune multigland syndrome, and multi-gland endocrine disorder Syndrome; paraneoplastic syndrome, such as neurological paraneoplastic syndrome; Lambert-Eaton myasthenia syndrome or Eaton-Lambert syndrome; Stiffman syndrome or systemic stiffness Genital syndrome; encephalomyelitis, e.g. allergic performance myelitis, encephalomyelitic allergy, and experimental allergic encephalomyelitis (EAE); myasthenia gravis, e.g. myasthenia gravis associated with thymoma Cerebellar degeneration; neuromuscular tone; ocular clonus or ocular clonus myoclonus syndrome (OMS); sensory neuropathy; multifocal motor neuropathy; Sheehan syndrome; hepatitis such as autoimmune hepatitis, chronic hepatitis, lupoid Hepatitis, giant cell hepatitis, chronic active hepatitis and autoimmune chronic active hepatitis; lymphoid interstitial pneumonia (LIP); obstructive bronchiolitis (non-transplant) vs NSIP; Guillain-Barre Syndrome; Berger's disease (IgA nephropathy); idiopathic IgA nephropathy; linear IgA dermatosis; acute neutrophilic dermatosis; subhorny pustular dermatosis; for example Primary biliary cirrhosis and pulmonary fibrosis; autoimmune bowel disease syndrome; Celiac or Coeliac disease; lipostool (gluten enteropathy); refractory sprue; idiopathic sprue; Globulinemia; amyotrophic lateral sclerosis (ALS; Louis Gehrig disease); ring arterial disease; autoimmune ear disease such as autoimmune inner ear disease (AIED); autoimmune hearing loss; Chondritis, e.g. refractory or relapsed or relapsing polychondritis; cytoproteinosis; Cogan syndrome/nonsyphilitic interstitial keratitis; Bell paralysis; Sweets disease/syndrome; autoimmune rosacea autoimmune; pain associated with shingles; amyloidosis; non-cancerous lymphocytosis; primary lymphedema, e.g. monoclonal B cell lymph Cytomegaly (e.g., benign monoclonal immunoglobulin and monoclonal gammopathy of undetermined significance (MGUS); peripheral neuropathy; channel disease, e.g., epilepsy, migraine, arrhythmia, muscle Disability, hemorrhoids, blindness, periodic paralysis, and CNS channel disease; autism; inflammatory myopathy; focal or segmental glomerulosclerosis (FSGS); endocrine ophthalmopathy; Autoimmune liver disease; fibromyalgia; multiple endocrine insufficiency; Schmidt syndrome; adrenalitis; gastric atrophy; presenile dementia; demyelinating diseases such as autoimmune demyelinating and chronic inflammatory Demyelinating polyneuropathy; Dressler syndrome; alopecia areata; complete alopecia; CREST syndrome (calcification, Raynaud phenomenon, hypoesophageal peristalsis, sclerotia, and telangiectasia); male And women's autoimmunity Infertility (e.g., anti-spermatozoan antibodies); mixed connective tissue disease; Chagas disease; rheumatic fever; recurrent miscarriage; farmer's lung; erythema erythema; postcardiotomy syndrome; Cushing syndrome; avian disease; allergic granulomatous vasculitis; benign cutaneous lymphocytic vasculitis; Alport syndrome; alveolitis, e.g. allergic alveolitis and fibroalveolaritis Interstitial pneumonia; transfusion reaction; leprosy; malaria; Samter syndrome; Caplan syndrome; endocarditis; endocardial myocardial fibrosis; diffuse interstitial pulmonary fibrosis; Interstitial mung fibrosis; pulmonary fibrosis; idiopathic pulmonary fibrosis; cystic fibrosis; endophthalmitis; persistent elevated erythema; fetal erythroblastosis; eosinophil fasciitis; Schulman syndrome Felty syndrome; flaresis; ciliary body inflammation, even Chronic ciliitis, metachronous ciliitis, iris ciliitis (acute or chronic), or Fuch ciliitis; Henoch-Schonlein purpura; sepsis; internal Toxemia; pancreatitis; thyroxicosis; Evan syndrome; autoimmune gland dysfunction; Sydenham chorea; post-streptococcal nephritis; obstructive thrombovasculitis; thyroid poisoning; dorsalis); choroiditis; giant cell polymyalgia; chronic hypersensitivity pneumonitis; dry keratoconjunctivitis; epidemic keratoconjunctivitis; idiopathic nephrotic syndrome; minimal change nephrosis; benign familial and ischemic perfusion disorders; Perfusion; Retinal autoimmunity; Joint inflammation; Bronchitis; Chronic obstructive airway/lung disease; Silicosis; Aphtha; Aphthous stomatitis; Arteriosclerotic disease; Aspermiogenese; Autoimmune hemolysis), Croglob Nchisho; Dupuis Trang (Dupuytren) contracture; lens hypersensitivity endophthalmitis (endophthalmia phacoanaphylactica); allergic enterocolitis; erythema nodosum leprosum; idiopathic facial paralysis; rheumatic fever; Hamman-Rich disease; sensory neuropathic hearing loss; paroxysmal hemoglobinuria (haemoglobinuria paroxysmatica); gonadal dysfunction; focal ileitis; leukopenia; infectious mononucleosis; Primary idiopathic myxedema; nephrosis; ophthalmia symphatica; orchitis granulomatosa; pancreatitis; acute polyneuropathy; gangrenous Pyoderma; Quervain thyroiditis; acquired spenic atrophy; nonmalignant thymoma; vitiligo; toxic shock syndrome; food poisoning; symptoms including T cell infiltration; leukocyte-adhesion deficiency; immune response associated with acute and delayed hypersensitivity mediated by cytokines and T-lymphocytes; symptoms including leukocyte leakage; multi-organ injury syndrome; mediated by antigen-antibody complex disease Anti-glomerular basement membrane antibody disease; allergic neuritis; autoimmune multiglandular endocrine insufficiency; ovitis; primary myxedema; autoimmune atrophic gastritis; interchangeable ophthalmitis; nephrotic syndrome; insulitis; multiglandular endocrine deficiency; polyglandular autoimmune syndrome type i (adult-onset idiopathic hypoparathyroidism: AOIH) cardiomyopathy such as dilated cardiomyopathy; acquired epidermolysis bullosa (EBA); hemochromatosis; myocarditis; nephrotic syndrome; primary sclerosing cholangitis; purulent or non-purulent sinusitis; Rhinosinitis; ethmoid sinusitis, frontal sinusitis, maxillary sinusitis, or sphenoid sinusitis; diseases related to eosinophils, such as eosinophilia, pulmonary wet eosinophilia, eosinophils Increased myalgia syndrome, Loffler syndrome, chronic eosinophil pneumonia Localized pulmonary eosinophilia, bronchopulmonary aspergillosis, aspergilloma, or granulomas including eosinophils; anaphylaxis; seronegative spondyloarthritides; multigland endocrine autoimmune disease; sclerosis chronic mucocutaneous glandosis; Bruton syndrome; Transient hypogammaglobulinemia in infancy; Wiskott-Aldrich syndrome; ataxic peripheral vasodilatation syndrome; vasodilatation; autoimmune diseases related to collagen disease, rheumatism, neurological diseases, lymphadenitis, decreased blood pressure response, vascular dysfunction, tissue damage, cardiovascular ischemia, hyperalgesia, renal ischemia, cerebral ischemia, and angiogenesis associated diseases; allergic hypersensitivity disease; glomerulonephritides; reperfusion injury; ischemic re-perfusion disorder; myocardial or other tissue reperfusion injury, Lymphoma bronchitis; inflammatory skin disease; dermatosis due to acute inflammatory component; multiple organ failure; bullous disease; nephrocortical necrosis; acute purulent meningitis or other central nervous system inflammatory disease; ocular and orbital inflammation diseases; granulocyte transfusion related syndromes; cytokine-induced toxicity; narcolepsy; acute severe inflammation; chronic refractory inflammation; pyelonephritis; arterial hyperplasia; peptic ulcer; valvitis; and endometriosis.

In some embodiments, an antibody provided herein can be used to treat a disease or injury associated with upregulation of protease-activated receptor 2 (PAR-2). In some embodiments, an antibody provided herein can be used to treat a cardiovascular disease or injury associated with upregulation of PAR-2. In some embodiments, the cardiovascular disease or injury is myocardial infarction. In some embodiments, the cardiovascular disease or injury is atherosclerosis. Examples of diseases associated with upregulation of PAR2 are provided, for example, in Heuberger, Dorothea M., and Reto A. Schuepbach. Thrombosis journal 17.1 (2019): 1-24 and Kagota, Satomi et al. BioMed research international vol. 2016 (2016): 3130496, the relevant disclosures of each of which are herein incorporated by reference.

In certain embodiments, an antibody provided herein can be used for the treatmen of cancer that is associated with inflammation. For example, an antibody provided herein may be administered for the treatment of CRS (cytokine releaase syndrome) after Car-T therapy. For example, a number of cancers associated with chronic inflammation, include colorectal, lung, mesothelioma, liver, esophageal, stomach, pancreas, gall bladder, ovarian/uterine, prostate, bladder, thyroid, salivary gland, mouth (squamous), and skin cancer, Hodgkin's disease/Non-Hodgkin's Lymphoma, and MALT (mucosa-associated lymphoid tissue). Additional examples of inflammation-associated cancers are provided in Coussens LM and Werb Z. Nature. 2002; 420(6917):860-867, which is incorporated by reference in its entirety.

13. Inflammation and Coagulopathies

Inflammation initiates clotting, decreases the activity of natural anticoagulant mechanisms and impairs the fibrinolytic system. Inflammatory cytokines are the major mediators involved in coagulation activation. Acute inflammation has been shown to results in systemic activation of coagulation. Systemic inflammation results in activation of coagulation, due to TF-mediated thrombin generation. Mediators in anticoagulation cascades (e.g. thrombomodulin) reduce cell responsiveness to inflammatory mediators and facilitate the neutralisation of some inflammatory mediators. Interactions between inflammation and coagulation are detailed in Esmon, C.T. British journal haematology 131.4 (2005): 417-430, which is incorporated by reference in its entirety.

Coagulopathy is a condition in which the body's ability to form clots is impaired. In patients it manifests as difficulty controlling bleeding, chronic bleeding and/or excessive bleeding, especially after a challenge such as injury, surgery or childbirth. Coagulopathy results from decreased hepatic synthesis of coagulation factors and the presence of disseminated intravascular coagulopathy (DIC), which is a process of accelerated consumption of coagulation factors and platelets. In DIC there is unregulated and excessive generation of thrombin and resultant consumption of coagulation factors (e.g., fibrinogen and factor VIII). Studies have shown that inflammatory activation in concert with microvascular thrombosis contributes to multiple organ failure in patients with severe infection and DIC. (See Levi, M., et al., Cardiovascular research 60.1 (2003): 26-39, which is incorporated by reference in its entirety).

The term “coagulopathy”, as used herein, refers to an increased haemorrhagic tendency which may be attributed to any qualitative or quantitative deficiency of any pro-coagulative component of the normal coagulation cascade, or any upregulation of fibrinolysis. Coagulopathies can be classified as acquired, congenital or iatrogenic. They can be diagnosed and tracked using measurement of prothrombin time (PT) and partial thromboplastin time (PTT). In certain embodiments, the antibodies provided herein are useful for the treatment of coagulopathies (e.g., acquired coagulopathies, congenital coagulopathies). Examples of coagulopathies that can be treated using the antibodies or ADCs provided herein include, but are not limited to, disseminated intravascular coagulopathy (DIC; consumptive coagulopathy), hemophilia A, hemophilia B, von Willebrand disease, idiopathic thrombocytopenia, deficiency of one or more contact factors such as factor X₁, factor XII, precallicrein, and high molecular weight kininogen (HMMK), a deficiency of one or more factors associated with significant clinical bleeding, such as factor V, factor VII, factor VIII, factor IX, factor X, factor XIII, factor II (hypoprothrombinemia) and von Willebrand factor, vitamin deficiency mine K, a disorder associated with fibrinogen, including afibrinogenemia, hypofibrinogenemia and dysphibrinogenemia, alpha2-antiplasmin deficiency and heavy bleeding, such as bleeding caused by liver disease, kidney disease, thrombocytopenia, platelet dysfunction, hematoma, hematoma, hematoma, hematoma trauma, hypothermia, bleeding during menstruation and pregnancy. In some embodiments, NASPs are used to treat congenital bleeding disorders, including hemophilia A, hemophilia B, and von Willebrand disease. Examples of acquired coagulation disorders, including factor VIII deficiency, von Willebrand factor, factor IX, factor V, factor X₁, factor XII and factor XIII deficiency, in particular disorders caused by inhibitors or an autoimmune reaction against blood coagulation factor, or hemostatic disorders caused by a disease or condition that leads to a decrease in the synthesis of coagulation factors. Additional examples of coagulopathies and methods for assessing changes in coagulopathy (e.g. due to treatment with an antibody) are provided in U.S. application Ser. No. 13/721,802, which is incorporated by reference in its entirety.

In certain embodiments, a subject suffers from a coagulopathy and treatment with an antibody or ADC provided herein reduces or ameliorates one or more symptoms of the coagulopathy.

14. Inflammatory Cytokines and Chemokines

In certain embodiments, upon administration to a subject, the antibody or ADC provided herein reduces the concentration of inflammatory cytokines or chemokines. Inflammatory cytokines or pro-inflammatory cytokines are types of signaling molecules (cytokines) that are secreted from immune cells (e.g., helper T cells (Th), macrophages) and promote inflammation. Inflammatory chemokines are small cytokines or signaling proteins that function mainly as chemoattractants for leukocytes, recruiting monocytes, neutrophils and other effector cells from the blood to sites of infection or tissue damage. They can be classified into four major subfamilies: CXC, CC, CX3C, and XC, all of which are bioactive by selectively binding to chemokine receptors located on the surface of target cells.

In certain embodiments, upon administration of an antibody or ADC provided herein, the antibody or ADC results in a reduction of inflammatory cytokines and chemokines relative to baseline levels or a different anti-inflammatory agent, wherein the inflammatory cytokines and chemokines are one or more of: IL-1α, IL-1β, IL-2, IL-4, IL-5, IL-6, IL-8, IL-10, IFNγ, GM-CSF, TNFα, CCL2, CCL3, CCL4, CCL5, CCL19, CCL20, CCL25, CXCL1, CXCL2, and CXCL10.

IL-1α (Interleukin 1 Alpha) is a member of the interleukin 1 cytokine family. It is a pleiotropic cytokine involved in various immune responses, inflammatory processes, and hematopoiesis. IL-1α is produced by monocytes and macrophages as a proprotein, which is proteolytically processed and released in response to cell injury, and thus induces apoptosis.

IL-1β (Interleukin 1 Beta) is a member of the interleukin 1 cytokine family and is produced by activated macrophages as a proprotein, which is proteolytically processed to its active form by caspase 1 (CASP1/ICE). IL-10 is an important mediator of the inflammatory response, and is involved in a variety of cellular activities, including cell proliferation, differentiation, and apoptosis. The induction of cyclooxygenase-2 (PTGS2/COX2) by this cytokine in the central nervous system (CNS) is found to contribute to inflammatory pain hypersensitivity.

IL-2 (Interleukin 2) is a cytokine that is important for the proliferation of T and B lymphocytes. IL-2 is part of the immune response to microbial infection, and discriminating between foreign (“non-self”) and “self”. In the thymus, where T cells mature, it prevents autoimmune diseases by promoting the differentiation of certain immature T cells into regulatory T cells, to prevent the destruction of healthy cells by T-cells. The targeted disruption of a similar gene in mice leads to ulcerative colitis-like disease, which suggests an essential role of this gene in the immune response to antigenic stimuli.

IL-4 (Interleukin 4) is a pleiotropic cytokine produced by activated T cells. One of the roles of the cytokine is the stimulation of activated B-cell and T-cell proliferation, and the differentiation of B cells into plasma cells. The presence of IL-4 in extravascular tissues promotes alternative activation of macrophages into M2 cells and inhibits classical activation of macrophages into M1 cells.

IL-5 (Interleukin 5) is a cytokine that acts as a growth and differentiation factor for both B cells and eosinophils, and it plays a major role in the regulation of eosinophil formation, maturation, recruitment and survival. Elevated IL-5 has been associated with the pathogenesis of eosinophil-dependent inflammatory diseases. (See Takatsu K., Proc Jpn Acad Ser B Phys Biol Sci. 2011; 87(8):463-485, which is incorporated by reference in its entirety).

IL-6 (Interleukin 6) is a cytokine that plays an important role in inflammation and B-cell maturation. It is an endogenous pyrogen capable of inducing fever in people with autoimmune diseases or infections. The protein is primarily produced at sites of acute and chronic inflammation, where it is secreted into the serum and induces a transcriptional inflammatory response through interleukin 6 receptor, alpha.

IL-8 (Interleukin 8, CXCL8 or C-X-C Motif Chemokine Ligand 8) is a chemokine-a member of the CXC chemokine family—and a major mediator of the inflammatory response and a potent angiogenic factor. It is primarily secreted by neutrophils, where it serves as a chemotactic factor by guiding the neutrophils to the site of infection.

IL-10 (Interleukin 10) is a cytokine produced primarily by monocytes. It has pleiotropic effects in immunoregulation and inflammation. It down-regulates the expression of Th1 cytokines, MHIC class II Ags, and costimulatory molecules on macrophages. It also enhances B cell survival, proliferation, and antibody production. It also blocks NF-kappa B activity, and is involved in the regulation of the JAK-STAT signaling pathway. Knockout studies in mice suggested the function of this cytokine as an essential immunoregulator in the intestinal tract. (See Schreiber, S., et al., Gastroenterology 108.5 (1995): 1434-1444, which is incorporated by reference in its entirety).

IFNγ (Interferon Gamma) is a soluble cytokine that is a member of the type II interferon class. It is a homodimer that binds to the interferon gamma receptor which triggers a cellular response to viral and microbial infections. Mutations in the gene that encodes IFNγ are associated with an increased susceptibility to pathogenic infections and to several autoimmune diseases.

GM-CSF (Granulocyte-macrophage colony-stimulating factor) is a cytokine secreted by macrophages, T cells, mast cells, natural killer cells, endothelial cells and fibroblasts. It is a monomeric glycoprotein that stimulates stem cells to produce granulocytes (neutrophils, eosinophils, and basophils) and monocytes. It also enhances neutrophil migration. It has been recognized as a target that, when blocked or inhibited, reduces inflammation.

TNFa (Tumor Necrosis Factor) is a multifunctional proinflammatory cytokine, mainly secreted by macrophages, that belongs to the tumor necrosis factor (TNF) superfamily. It can bind to, and thus functions through its receptors TNFRSF1A/TNFR1 and TNFRSF1B/TNFBR. TNFa is involved in the regulation of a wide spectrum of biological processes including cell proliferation, differentiation, apoptosis, lipid metabolism, and coagulation.

CCL2 (C-C Motif Chemokine Ligand 2) is a member of the CC chemokine family characterized by two adjacent cysteine residues. CCL2 displays chemotactic activity for monocytes and basophils but not for neutrophils or eosinophils. It has been implicated in the pathogenesis of diseases characterized by monocytic infiltrates, like psoriasis, rheumatoid arthritis and atherosclerosis.

CCL3 (C-C Motif Chemokine Ligand 3 or macrophage inflammatory protein 1-alpha) is a member of the CC chemokine family. It plays a role in inflammatory responses through binding to the receptors CCR1, CCR4 and CCR5. It is a chemoattractant for macrophages, monocytes and neutrophils.

CCL4 (C-C Motif Chemokine Ligand 4) is a mitogen-inducible monokine secreted by neutrophils, monocytes, B cells, T cells, fibroblasts, endothelial cells, and epithelial cells, and is one of the major HIV-suppressive factors produced by CD8+ T-cells. The encoded protein is secreted and has chemokinetic and inflammatory functions.

CCL5 (C-C Motif Chemokine Ligand 5) is a member of the CC chemokine family characterized by two adjacent cysteine residues. This chemokine functions as a chemoattractant for blood monocytes, memory T helper cells and eosinophils. It causes the release of histamine from basophils and activates eosinophils. This cytokine is one of the major HIV-suppressive factors produced by CD8+ cells.

CCL19 (C-C Motif Chemokine Ligand 19) is a member of the CC chemokine family characterized by two adjacent cysteine residues. It plays a role in normal lymphocyte recirculation and homing. It also plays an important role in trafficking of T cells in thymus, and in T cell and B cell migration to secondary lymphoid organs.

CCL20 (C-C Motif Chemokine Ligand 20) is a member of the CC chemokine family characterized by two adjacent cysteine residues. It displays chemotactic activity for lymphocytes and can repress proliferation of myeloid progenitors.

CCL25 (C-C Motif Chemokine Ligand 25) are cytokines that display a chemotactic activity for dendritic cells, thymocytes, and activated macrophages but is inactive on peripheral blood lymphocytes and neutrophils.

CXCL1 (C-X-C Motif Chemokine Ligand 1) is a member of the CXC subfamily of chemokines that signals through the G-protein coupled receptor, CXC receptor 2. CXCL1 is expressed by macrophages, neutrophils and epithelial cells and has neutrophil chemoattractant activity. Aberrant expression of this protein is associated with the growth and progression of certain tumors.

CXCL2 (C-X-C Motif Chemokine Ligand 2 or macrophage inflammatory protein 2-alpha) is chemokine in the CXC subfamily that is expressed at sites of inflammation. It is secreted by monocytes and macrophages and is chemotactic for polymorphonuclear leukocytes and hematopoietic stem cells.

CXCL10 (C-X-C Motif Chemokine Ligand 10) is a chemokine in the CXC subfamily. It is a ligand for the receptor CXCR3. Binding of this protein to CXCR3 results in pleiotropic effects, including stimulation of monocytes, natural killer and T-cell migration, and modulation of adhesion molecule expression.

Non-limiting examples of inflammatory cytokines and chemokines are provided in Turner, M. D., et al. Biochimica et Biophysica Acta (BBA)—Molecular Cell Research 1843.11 (2014): 2563-2582, which is incorporated by reference in its entirety.

The inflammatory cytokines and chemokines described herein can be measured, for example, using immunohistochemistry, ELISA, MSD-ECLA, Olink panels (e.g. custom Olink panels; Olink Proteomics, Uppsala, Sweden), or Luminex Multiplex Assay. Alternatively, the expression levels for inflammatory cytokines in blood samples can be measured using RT-PCR.

15. Comparator Therapies for Treatment of Inflammatory Diseases

The antibodies and ADCs of the present disclosure are useful for the treatment of inflammatory diseases. In certain embodiments, the antibodies and ADCs provided herein mitigate or reduce the symptoms or indicators of inflammatory disease to a greater extent than comparator therapies, other anti-inflammatory therapeutics (also referred to as anti-inflammatory agents). These anti-inflammatory agents are alternative therapies that are known or indicated for the treatment of the inflammatory diseases contemplated herein. For example, in certain embodiments, the comparator anti-inflammatory agents are selected from any one of: non-steroidal anti-inflammatory drugs (NSAIDs), steroidal anti-inflammatory drugs, beta-agonists, anticholinergic agents, antihistamines, and methyl xanthines. In certain embodiments, the comparator anti-inflammatory agents are IL-6 inhibitors (soluble IL-6 and IL-6R), GM-CSF inhibitors, TNFa inhibitors, anti-IL-1α, dexamethasone, chemokine and chemokine receptor antagonists or JAK inhibitors. In certain embodiments, the comparator anti-inflammatory agent is cyclosporine.

Due to the role of IL-6 in inflammation and autoimmune disease (discussed supra), IL-6 is recognized as a viable target for autoimmune diseases. Non-limiting examples of IL-6 inhibitors include: anti-IL-6 antibodies, anti-IL-6 receptor antibodies, anti-gp130 antibodies, IL-6 variants, IL-6 receptor variants, soluble, and partial peptides of IL-6 or IL-6 receptor, and low molecular weight compounds and protons (for example, C326 Avimer (Nature Biotechnology (2005) 23:1556-61, which is incorporated by reference in its entirety)) showing similar activities. There are high levels of IL-6 in the synovium and serum of patients having rheumatoid arthritis (RA). Recent studies have shown significant efficacy in the treatment of RA with IL-6 inhibitors. (See Hennigan S., and Kavanaugh A. Ther Clin Risk Manag. 2008; 4(4):767-775, which is incorporated by reference in its entirety). Examples of available IL-6 inhibitor drugs include tocilizumab (RoActemra, Roche) and sarilumab (Kevzara, Sanofi).

Tocilizumab is a recombinant humanized monoclonal antibody IL-6 receptor inhibitor having the following light chain and heavy chain sequences:

Tocilizumab light chain:
(SEQ ID NO: 930)
DIQMTQSPSSLSASVGDRVTITCRASQDISSYLNWYQQKPGKAPKLLIYY
TSRLHSGVPSRFSGSGSGTDFTFTISSLQPEDIATYYCQQGNTLPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Tocilizumab heavy chain:
(SEQ ID NO: 931)
QVQLQESGPGLVRPSQTLSLTCTVSGYSITSDHAWSWVRQPPGRGLEWIG
YISYSGITTYNPSLKSRVTMLRDTSKNQFSLRLSSVTAADTAVYYCARSL
ARTTAMDYWGQGSLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG

Sarilumab is a fully human anti-IL-6R monoclonal IgG1 antibody that binds to both membrane bound and soluble interleukin 6 (IL-6) receptor forms. It has the following light chain and heavy chain sequences:

Sarilumab Light chain:
(SEQ ID NO: 932)
DIQMTQSPSSVSASVGDRVTITCRASQGISSWLAWYQQKPGKAPKLLIYG
ASSLESGVPSRFSGSGSGTDFTLTISSLQPEDFASYYCQQANSFPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Sarilumab Heavy chain:
(SEQ ID NO: 933)
EVQLVESGGGLVQPGRSLRLSCAASRFTFDDYAMHWVRQAPGKGLEWVSG
ISWNSGRIGYADSVKGRFTISRDNAENSLFLQMNGLRAEDTALYYCAKGR
DSFDIWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK

Due to the pro-inflammatory function of GM-CSF, therapies have been developed to target and inhibit the cytokine. Non-limiting examples of antibodies, antibody fragments that target GM-CSF, and other GM-CSF antagonists are provided in U.S. application Ser. Nos. 16/442,779 and 11/944,162, each of which is incorporated by reference in its entirety.

TNFα inhibitors are agents that interfere with the activity of TNFα (described supra). They include, without limitation, each of the anti-TNFα human antibodies and antibody portions described herein as well as those described in U.S. Pat. Nos. 6,090,382; 6,258,562; 6,509,015, and in U.S. patent application Ser. No. 09/801,185 (now U.S. Pat. No. 7,223,394) and 10/302,356, each of which is incorporated by reference in its entirety. In one embodiment, the TNFα inhibitor used in the invention is an anti-TNFα antibody, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab or Simponi; Medarex and Centocor, see International Application No. PCT/US2001/024785, which is incorporated by reference in its entirety), and adalimumab (Humira® Abbott Laboratories, a human anti-TNF mAb, described as D2E7 in U.S. Pat. No. 6,090,382, incorporated by reference in its entirety). Additional TNF antibodies which can be used in the invention are described in U.S. Pat. Nos. 6,593,458; 6,498,237; 6,451,983; and 6,448,380, each of which is incorporated by reference in its entirety. In another embodiment, the TNFα inhibitor is a TNF fusion protein, e.g., etanercept (Enbrel®, Amgen; described in International Application No. PCT/US1990/004001, incorporated by reference in its entirety). In another embodiment, the TNFα inhibitor is a recombinant TNF binding protein (r-TBP-I) (Serono). Another example of a TNFα inhibitor is certolizumab pegol (Cimzia).

Certolizumab pego is a pegylated monoclonal antibody against the tumor necrosis factor-alpha (TNF-alpha). Exemplary sequences for the heavy and light chains are provided below:

Certolizumab pegol light chain:
(SEQ ID NO: 934)
DIQMTQSPSSLSASVGDRVTITCKASQNVGTNVAWYQQKPGKAPKALIYS
ASFLYSGVPYRFSGSGSGTDFTLTISSLQPEDFATYYCQQYNIYPLTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Certolizumab Pegol heavy chain:
(SEQ ID NO: 935)
EVQLVESGGGLVQPGGSLRLSCAASGYVFTDYGMNWVRQAPGKGLEWMGW
INTYIGEPIYADSVKGRFTFSLDTSKSTAYLQMNSLRAEDTAVYYCARGY
RSYAMDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCAA

IL-1α inhibitors interfere with the activity of IL-1α (described supra). Non-limiting examples of IL-1α inhibitors include Bermekimab (MABp1 or Xilonix) and Rilonacept.

Bermekimab (MABp1 or Xilonix) is a human monoclonal antibody of IgG1k isotype targeting Interleukin 1 alpha. Examplary sequences for the Bermekimab heavy and light chains are provided below:

Bermekimab Heavy chain:
(SEQ ID NO: 936)
QVQLVESGGGVVQPGRSLRLSCTASGFTFSMFGVHWVRQAPGKGLEWVAA
VSYDGSNKYYAESVKGRFTISRDNSKNILFLQMDSLRLEDTAVYYCARGR
PKVVIPAPLAHWGQGTLVTFSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
Bermekimab Light chain:
(SEQ ID NO: 937)
DIQMTQSPSSVSASVGDRVTITCRASQGISSWLAWYQQKPGKAPKLLIYE
ASNLETGVPSRFSGSGSGSDFTLTISSLQPEDFATYYCQQTSSFLLSFGG
GTKVEHKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Rilonacept is a dimeric fusion protein that functions as an interleukin 1 inhibitor and is used in the treatment of CAPS, also known as cryopyrin-associated periodic syndromes, including familial cold auto-inflammatory syndrome (FCAS) and Muckle-Wells Syndrome (MWS). IL-1α is one of its targets. An exemplary sequence for rilonacept is provided below:

(SEQ ID NO: 938)
SERCDDWGLDTMRQIQVFEDEPARIKCPLFEHFLKFNYSTAHSAGLTLIW
YWTRQDRDLEEPINFRLPENRISKEKDVLWFRPTLLNDTGNYTCMLRNTT
YCSKVAFPLEVVQKDSCFNSPMKLPVHKLYIEYGIQRITCPNVDGYFPSS
VKPTITWYMGCYKIQNFNNVIPEGMNLSFLIALISNNGNYTCVVTYPENG
RTFHLTRTLTVKVVGSPKNAVPPVIHSPNDHVVYEKEPGEELLIPCTVYF
SFLMDSRNEVWWTIDGKKPDDITIDVTINESISHSRTEDETRTQILSIKK
VTSEDLKRSYVCHARSAKGEVAKAAKVKQKVPAPRYTVEKCKEREEKIIL
VSSANEIDVRPCPLNPNEHKGTITWYKDDSKTPVSTEQASRIHQHKEKLW
FVPAKVEDSGHYYCVVRNSSYCLRIKISAKFVENEPNLCYNAQAIFKQKL
PVAGDGGLVCPYMEFFKNENNELPKLQWYKDCKPLLLDNIHFSGVKDRLI
VMNVAEKHRGNYTCHASYTYLGKQYPITRVIEFITLEENKPTRPVIVSPA
NETMEVDLGSQIQLICNVTGQLSDIAYWKWNGSVIDEDDPVLGEDYYSVE
NPANKRRSTLITVLNISEIESRFYKHPFTCFAKNTHGIDAAYIQLIYPVT
NSGDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

Dexamethasone, or MK-125, is a corticosteroid fluorinated at position 9 used to treat endocrine, rheumatic, collagen, dermatologic, allergic, ophthalmic, gastrointestinal, respiratory, hematologic, neoplastic, edematous, and other conditions. The exemplary structure for Dexamethasone is provided below:

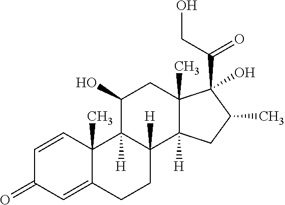

As used herein, the terms “chemokine antagonist” and “chemokine receptor antagonist” refer to a drug or molecule that inhibits, decreases, abrogates, or blocks binding of a chemokine to one or more of its cognate receptors. Non-limiting examples of chemokine antagonists and chemokine receptor antagonists are provided in U.S. application Ser. Nos. 15/759,886 and 10/996,353, each of which is incorporated by reference in its entirety.

JAK inhibitors function by inhibiting the activity of one or more of the Janus kinase family of enzymes (JAK1, JAK2, JAK3, TYK2), thereby interfering with the JAK-STAT signaling pathway. The Janus Kinase (JAK) family plays an important role in cytokine dependent regulation of the proliferation and action of cells involved in immune responses. Non-limiting examples of JAK inhibitors are provided in US Application Nos. 12/401,348 and international application No. PCT/US2017/025117, each of which is incorporated by reference in its entirety.

AZD1480 is a potent, adenosine triphosphate competitive, small-molecule inhibitor of JAK2 kinase. It has been used in trials studying the treatment of Solid Malignancies, Post-Polycythaemia Vera, Primary Myelofibrosis (PMF), and Essential Thrombocythaemia Myelofibrosis. It has been shown to suppress growth, survival, as well as FGFR3 and STAT3 signaling, and downstream targets including Cyclin D2 in human multiple myeloma cells. (See Scuto, Anna, et al. Leukemia 25.3 (2011): 538-550, which is incorporated by reference in its entirety). The exemplary structure for AZD1480 is provided below:

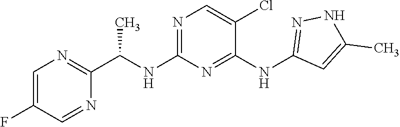 [SMILES: Cc1cc(Nc2nc(N[C@@H](C)c3ncc(F)cn3)ncc2Cl)n[nH]1]

Cyclosporine (CsA) is a calcineurin inhibitor known for its immunomodulatory properties that prevent organ transplant rejection and treat various inflammatory and autoimmune conditions. The exemplary structural for cyclosporine is provide below:

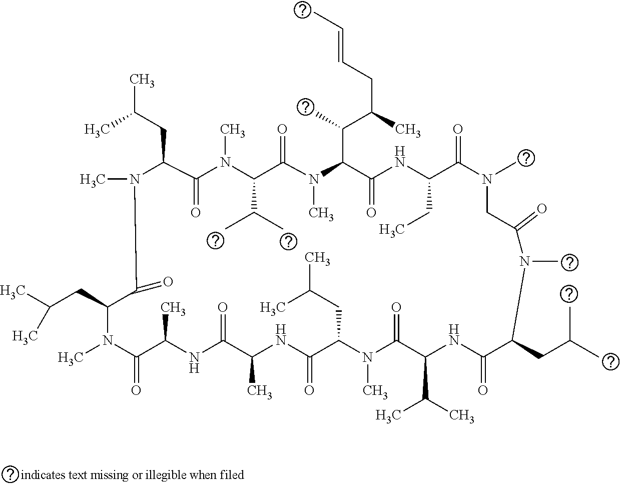

Non-limiting examples of anti-inflammatory agents include non-steroidal anti-inflammatory drugs (NSAIDs), steroidal anti-inflammatory drugs, beta-agonists, anticholinergic agents, antihistamines (e.g., ethanolamines, ethylenediamines, piperazines, and phenothiazine), and methyl xanthines. Examples of NSAIDs include, but are not limited to, aspirin, ibuprofen, salicylates, acetominophen, celecoxib, diclofenac, etodolac, fenoprofen, indomethacin, ketoralac, oxaprozin, nabumentone, sulindac, tolmentin, rofecoxib, naproxen, ketoprofen and nabumetone. Such NSAIDs function by inhibiting a cyclooxgenase enzyme (e.g., COX-1 and/or COX-2). Examples of steroidal anti-inflammatory drugs include, but are not limited to, glucocorticoids, dexamethasone, cortisone, hydrocortisone, prednisone, prednisolone, triamcinolone, azulfidine, and eicosanoids such as prostaglandins, thromboxanes, and leukotrienes.

16. Combination Therapies

In some embodiments, an antibody or ADC provided herein is administered with at least one additional therapeutic agent. Any suitable additional therapeutic agent may be administered with an antibody or ADC provided herein. In some aspects, the additional therapeutic agent is selected from radiation, a cytotoxic agent, a chemotherapeutic agent, a cytostatic agent, an anti-hormonal agent, an immunostimulatory agent, an immunosuppressive agent, an anti-inflammatory agent, an anti-angiogenic agent, and combinations thereof.

The additional therapeutic agent may be administered by any suitable means. In some embodiments, an antibody or ADC provided herein and the additional therapeutic agent are included in the same pharmaceutical composition. In some embodiments, an antibody or ADC provided herein and the additional therapeutic agent are included in different pharmaceutical compositions.

In embodiments where an antibody or ADC provided herein and the additional therapeutic agent are included in different pharmaceutical compositions, administration of the antibody or ADC can occur prior to, simultaneously, and/or following, administration of the additional therapeutic agent.

17. Diagnostic Methods

Also provided are methods for detecting the presence of TF on cells from a subject. Such methods may be used, for example, to predict and evaluate responsiveness to treatment with an antibody or ADC provided herein.

In some embodiments, the method can be used to detect TF in a subject having or suspected of having an inflammatory disease. In some embodiments, the methods comprise (a) receiving a sample from the subject; and (b) detecting the presence or the level of TF in the sample by contacting the sample with the antibody provided herein. In some embodiments, the methods comprise (a) administering to the subject the antibody provided herein; and (b) detecting the presence or the level of TF in the subject. In some embodiments, the inflammatory disease is any one of colitis, inflammatory bowel disease, arthritis, acute lung injury (ALI), acute respiratory distress syndrome (ARDS), and Respiratory Syncytial Virus (RSV). In some embodiments, inflammatory disease involves vascular inflammation.

In some embodiments, the methods comprise (a) administering to the subject the ADC provided herein; and (b) detecting the presence or the level of TF in the subject. In some embodiments, the inflammatory disease is any one of colitis, inflammatory bowel disease, arthritis, acute lung injury (ALI), acute respiratory distress syndrome (ARDS), and Respiratory Syncytial Virus (RSV).

In some embodiments, the antibody provided herein is conjugated with a fluorescent label. In some embodiments, the antibody provided herein is conjugated with a radioactive label. In some embodiments, the antibody provided herein is conjugated with an enzyme label.

In some embodiments, the ADC provided herein comprises a fluorescent label. In some embodiments, the ADC provided herein comprises a radioactive label. In some embodiments, the ADC provided herein comprises an enzyme label.

In some embodiments, the relative amount of TF expressed by such cells is determined. The fraction of cells expressing TF and the relative amount of TF expressed by such cells can be determined by any suitable method. In some embodiments, flow cytometry is used to make such measurements. In some embodiments, fluorescence assisted cell sorting (FACS) is used to make such measurement.

18. Kits

Also provided are kits comprising the antibodies or ADCs provided herein. The kits may be used for the treatment, prevention, and/or diagnosis of a disease or disorder, as described herein.

In some embodiments, the kit comprises a container and a label or package insert on or associated with the container. Suitable containers include, for example, bottles, vials, syringes, and IV solution bags. The containers may be formed from a variety of materials, such as glass or plastic. The container holds a composition that is by itself, or when combined with another composition, effective for treating, preventing and/or diagnosing a disease or disorder. The container may have a sterile access port. For example, if the container is an intravenous solution bag or a vial, it may have a port that can be pierced by a needle. At least one active agent in the composition is an antibody or ADC provided herein. The label or package insert indicates that the composition is used for treating the selected condition.

In some embodiments, the kit comprises (a) a first container with a first composition contained therein, wherein the first composition comprises an antibody or ADC provided herein; and (b) a second container with a second composition contained therein, wherein the second composition comprises a further therapeutic agent. The kit in this embodiment of the invention may further comprise a package insert indicating that the compositions can be used to treat a particular condition.

Alternatively, or additionally, the kit may further comprise a second (or third) container comprising a pharmaceutically-acceptable excipient. In some aspects, the excipient is a buffer. The kit may further include other materials desirable from a commercial and user standpoint, including filters, needles, and syringes.

### EXAMPLES

The following are examples of methods and compositions of the invention. It is understood that various other embodiments may be practiced, given the general description provided herein.

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures andMolecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Example 1: Generation of TF Antibodies

Human, cynomolgus monkey, and mouse TF extracellular domain (ECD) fragments were expressed as C-terminal His or Fcγ fragment fusions. Expi293 cells (ThermoFisher Scientific, Waltham, MA, USA) were transiently transfected as recommended by the manufacturer with pcDNA3.1V5-HisA (ThermoFisher Scientific) encoding human, cynomolgus, or mouse TF ECD-His6 (TF-His; SEQ ID NOs:811, 815, and 819, respectively) or pFUSE-hIgGl-Fc (Invivogen, San Diego, CA, USA) encoding human, cynomolgus or mouse TF ECD-Fc (TF-Fc; SEQ ID NOs:812, 816, and 820, respectively). For the His-tagged proteins, cell culture supernatants cleared from cells by centrifugation were preconditioned with 330 mM sodium chloride and 13.3 mM imidazole. Using recommended procedures, the TF-His6 and TF-Fc proteins were purified by affinity chromatography with a HisTrap HP and MabSelect SuRe column (GE Healthcare Bio-Sciences, Marlborough, MA, USA), respectively. FVII-Fc expressed in Expi293 was purified by affinity chromatography with a MabSelect SuRe column, followed by size exclusion chromatography. The TF-His6 and TF-Fc proteins were biotinylated with a 15× molar excess of Sulfo-NHS-SS-biotin as recommended (ThermoFisher Scientific). The non-labeled and biotinylated proteins were further purified by size exclusion chromatography using a Superdex 200 Increase 10/300 column (GE Healthcare Bio-Sciences).

Human antibodies against human TF were generated by Adimab™ yeast-based antibody presentation using the biotinylated recombinant TF proteins as screening antigens, as described below. All antibodies against human TF were evaluated for cross-reactivity with cynomolgus monkey and mouse TF. The binding activity of the antibodies to human, cynomolgus monkey, and mouse TF is shown in Table 5.

I. Library Interrogation and Selection Methodology for Isolation of Anti-TF Antibodies

Naive Library Selections

Eight naïve human synthetic yeast libraries each of ˜10⁹ diversity were designed, generated, and propagated as described previously (see, e.g., WO2009036379; WO2010105256; WO2012009568; Xu et al., Protein Eng Des Sel., 2013, 26(10):663-70). Eight parallel selections were performed, using the eight naive libraries for monomeric human TF selections.

For the first two rounds of selection, a magnetic bead sorting technique utilizing the Miltenyi MACS system was performed, essentially as described (Siegel et al., JImmunol Methods, 2004, 286(1-2):141-53). Briefly, yeast cells (˜10¹⁰ cells/library) were incubated with 10 nM of biotinylated human TF Fc-fusion antigen for 15 min at room temperature in FACS wash buffer PBS with 0.1% BSA. After washing once with 50 mL ice-cold wash buffer, the cell pellet was resuspended in 40 mL wash buffer, and 500 μl Streptavidin MicroBeads (Miltenyi Biotec, Bergisch Gladbach, Germany; Cat #130-048-101) were added to the yeast and incubated for 15 min at 4° C. Next, the yeast were pelleted, resuspended in 5 mL wash buffer, and loaded onto a MACS LS column (Miltenyi Biotec, Bergisch Gladbach, Germany; Cat. #130-042-401). After the 5 mL was loaded, the column was washed 3 times with 3 mL FACS wash buffer. The column was then removed from the magnetic field, and the yeast were eluted with 5 mL of growth media and then grown overnight.

Subsequent to the two rounds of MACS, the following four rounds of sorting were performed using flow cytometry (FACS). For the first round of FACS, approximately 5×107 yeast were pelleted, washed three times with wash buffer, and incubated with 10 nM of each the biotinylated Fc-fusion proteins of mouse and/or cynomolgus TF antigen for 10⁻¹⁵ min at room temperature. Yeast were then washed twice and stained with LC-FITC diluted 1:100 (Southern Biotech, Birmingham, Alabama; Cat #2062-02) and either SA-633 (Life Technologies, Grand Island, NY; Cat #S21375) diluted 1:500, or EA-PE (Sigma-Aldrich, St Louis; Cat #E4011) diluted 1:50, secondary reagents for 15 min at 4° C. After washing twice with ice-cold wash buffer, the cell pellets were resuspended in 0.4 mL wash buffer and transferred to strainer-capped sort tubes. Sorting was performed using a FACS ARIA sorter (BD Biosciences), and sort gates were determined to select for TF binding. The mouse- and cyno-selected populations from the first round of FACS were grown out and expanded through sub-culturing in selective media. The second, third, and fourth rounds of FACS involved positive sorts to enrich for TF binders and/or negative sorts to decrease the number of non-specific binders using soluble membrane proteins from CHO cells (see, e.g., WO2014179363 and Xu et al., PEDS, 2013, 26(10):663-70). After the final round of sorting, yeast were plated and sequenced.

Affinity Maturation of Clones Identified in Naive Selections

Heavy chains from the naive outputs (described above) were used to prepare light chain diversification libraries, which were then used for additional selection rounds. In particular, heavy chain variable regions were extracted from the fourth naive selection round outputs and transformed into a light chain library with a diversity of 1×10⁶.

The first of selection round utilized Miltenyi MACS beads and 10 nM biotinylated human TF Fc-fusion as antigen. Subsequent to the MACS bead selections, three rounds of FACS sorting were performed as described above using cynomolgus and mouse Fc-fusion TF at 10 nM or either biotinylated Fc-fusion TF antigens or biotinylated monomeric HIS-forms of human, mouse or cynomolgus TF. Individual colonies from each FACS selection round were sequenced.

Optimization of Leads Identified from Naive or Light Chain Diversification Selections

Optimization of lead clones was carried out utilizing three maturation strategies: diversification of CDR-H1 and CDR-H2; diversification of CDR-H3 following CDR-H1 and CDR-H2 diversity pool optimization; and diversification of CDR-L3 within selected CDR-L1 and CDR-L2 diversity pools.

CDR-H1 and CDR-H2 selection: The CDR-H3s from clones selected from either naive or light chain diversification procedure were recombined into a premade library with CDR-H1 and CDR-H2 variants of a diversity of 1×10⁻⁸ and selections were performed using biotinylated Fc-fusion cynomolgus TF antigen, biotinylated cynomolgus HIS-TF antigen, and/or biotinylated human HIS-TF. Affinity pressures were applied by using decreasing concentrations of biotinylated HIS-TF antigens (down to 1 nM) under equilibrium conditions at room temperature.

CDR-H3/CDR-H1/CDR-H2 selections: Oligos were ordered from IDT which comprised the CDR-H3 as well as a homologous flanking region on either side of the CDR-H3. Amino acid positions in the CDR-H3 were variegated via NNK diversity at two positions per oligo across the entire CDR-H3. The CDR-H3 oligos were double-stranded using primers which annealed to the flanking region of the CDR-H3. The remaining FR1 to FR3 of the heavy chain variable region was amplified from pools of antibodies with improved affinity that were isolated from the CDR-H1 and CDR-H2 diversities selected above. The library was then created by transforming the double stranded CDR-H3 oligo, the FR1 to FR3 pooled fragments, and the heavy chain expression vector into yeast already containing the light chain of the parent. Selections were performed as during previous cycles using FACS sorting. FACS rounds assessed non-specific binding, species cross-reactivity, and affinity pressure, and sorting was performed to obtain populations with the desired characteristics. Affinity pressures for these selections were performed as described above in the CDR-H1 and CDR-H2 selection.

CDR-L3/CDR-L1/CDR-L2 selections: Oligos were ordered from IDT which comprised the CDR-L3 as well as a homologous flanking region on either side of the CDR-L3. Amino acid positions in the CDR-L3 were variegated via NNK diversity at one position per oligo across the entire CDR-L3. The CDR-L3 oligos were double-stranded using primers which annealed to the flanking region of the CDR-L3. The remaining FR1 to FR3 of the light chain variable region was amplified from pools of antibodies with improved affinity that were isolated from the CDR-L1 and CDR-L2 diversities selected above. The library was then created by transforming the double stranded CDR-L3 oligo, the FR1 to FR3 pooled fragments, and the light chain expression vector into yeast already containing the heavy chain of the parent. Selections were performed as during previous cycles using FACS sorting. FACS rounds assessed non-specific binding, species cross-reactivity, and affinity pressure, and sorting was performed to obtain populations with the desired characteristics. Affinity pressures included titrations as well as incorporation of the parental Fab in antigen pre-complexation.

II. IgG and Fab Production and Purification

In order to produce sufficient amounts of selected antibodies for further characterization, the yeast clones were grown to saturation and then induced for 48 h at 30° C. with shaking. After induction, yeast cells were pelleted and the supernatants were harvested for purification. IgGs were purified using a Protein A column and eluted with acetic acid, pH 2.0. Fab fragments were generated by papain digestion and purified over CaptureSelect IgG-CH1 affinity matrix (LifeTechnologies, Cat #1943200250).

### Example 2: Influence of Anti-TF Antbody in DSS-Colitis Model

An in vivo study was conducted to determine the effects of an anti-TF antibody, (e.g., 43D8) on inflammatory endpoints in a colitis model. The 43D8 clone was used in this and following examples as a surrogate for the other anti-TF antibodies described herein because it is cross-reactive with mouse TF and binds to mouse TF with a high affinity. See, for example, Table 5.

In a colitis model, the administration of dextran sulfate sodium (DSS) causes colitis-like pathologies due to toxicity to colonic epithelial cells, which leads to a compromised mucosal function and infiltration of neutrophils, macrophages and lymphocytes. It results in loss of epithelial barrier function, secretion of proinflammatory cytokines and chemokines, and the influx of cells with cytotoxic potential, such as neutrophils and inflammatory macrophages. It is not considered a T-cell-mediated process in the art.

On Day 0 of the study, 8-12 week old male C57BL/6 mice received either sterile water ad libitum (Group 1, n=5) or 2.5% (w/v) DSS dissolved into sterile water ad libitum (Groups 2-5, n=10 mice per group). On Day 0 and Day 4, the mice from Group 2, 4, and 5 received the following doses (intravenous route):
Group 2; vehicle (PBS)
Group 4; test article at 3 mg/kg
Group 5; test article at 10 mg/kg
   The test article was anti-TF antibody 43D8.

Also starting on Day 0 to Day 10, the mice in Group 3 were treated once daily by oral gavage with the positive control cyclosporine (CsA) at 80 mg/kg (Neoral). On Day 8, all animals received sterile water for the remainder of the experiment and were euthanized on day 10.

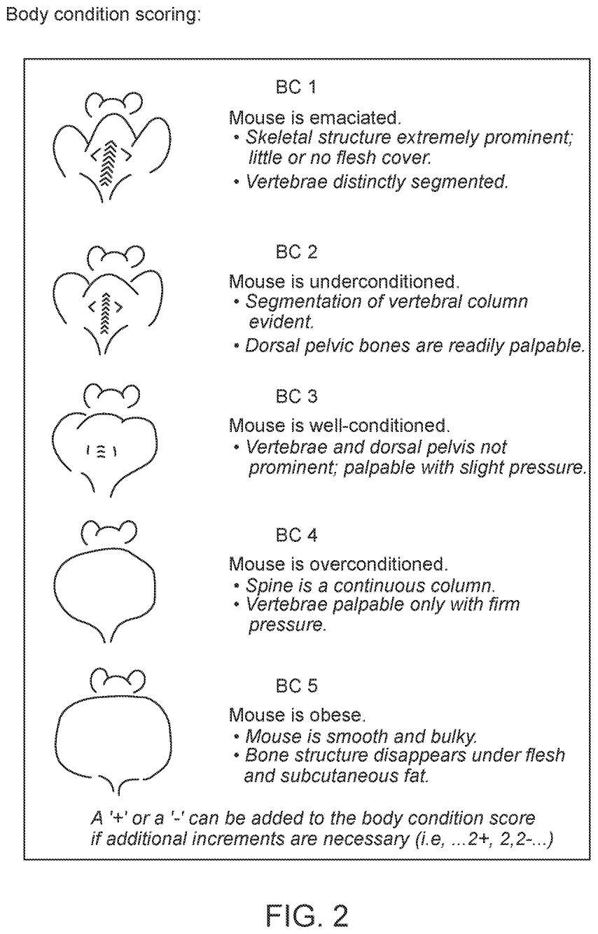

Throughout the study clinical observations were made daily. Body weight was measured and recorded daily (from Day 0 to Day 10). Body condition was also evaluated visually daily using the scoring system illustrated in FIG. 2. The stool consistency was determined qualitatively and blood in stool was measured daily using a hemoccult stool bleeding test. Tables 50, 60, and 61 illustrate the scoring systems used for assessing stool consistency, stool blood (occult blood) and changes in weight relative to baseline levels (Day 0). The stool consistency score, stool blood score and weight score were combined to provide a disease activity index for each subject at the time of measurement. Table 62 shows the compounded scoring system that determined the disease activity index.

TABLE 50
Stool Consistency Score

| Score | 0 | 1 | 2 | 3 |
|---|---|---|---|---|
| Observation | normal | moist/sticky stool | soft stool | diarrhea |

TABLE 60
Stool Blood Score

| Score | 0 | 1 | 2 | 3 |
|---|---|---|---|---|
| Hemoccult | negative | positive | positive | gross blood |
| test | hemoccult | hemoccult | hemoccult | observable |
| result | test, | test | test | on the |
| | no blood | in >30 seconds | in <30 seconds | slide |

TABLE 61
Weight Score

| Score | 0 | 1 | 2 | 3 | 4 |
|---|---|---|---|---|---|
| Weight loss | 0% | 1-5% | 6-10% | 11-15% | 16% or |
| relative to | | | | | higher |
| baseline | | | | | |

TABLE 62
Disease Activity Index (DAI) score, which was a combination of Stool
Consistency Score + Stool Blood Score + Weight Score.

| | Stool Consistency | | |
|---|---|---|---|
| Score | Score | Stool Blood Score | Weight Loss |
| 0 | Normal | negative hemoccult | 0% |
| | | test, no blood | |
| 1 | moist/sticky stool | positive hemoccult | 1-5% |
| | | test in >30 seconds | |
| 2 | soft stool | positive hemoccult | 6-10% weight |
| | | test in <30 seconds | loss |
| 3 | diarrhea | gross blood | 11-15% weight |
| | | observable on the | loss |
| | | slide | |
| 4 | N/A | N/A | 16% or higher |

Following euthanasia, the animal was measured (length determined) and weighed. Weight/length ratio was calculated for each animal. The animals were dissected and the weight of their spleens was determined. For each animal, the colon was “swiss-rolled” and placed in 10% neutral-buffered formalin (NBF) for 24 hours, followed by 70% ethanol. Fixed colon samples was processed in house. The samples were embedded in paraffin, sectioned at 5 microns, and slides stained with hematoxylin and eosin (H&E) for histologically analysis.

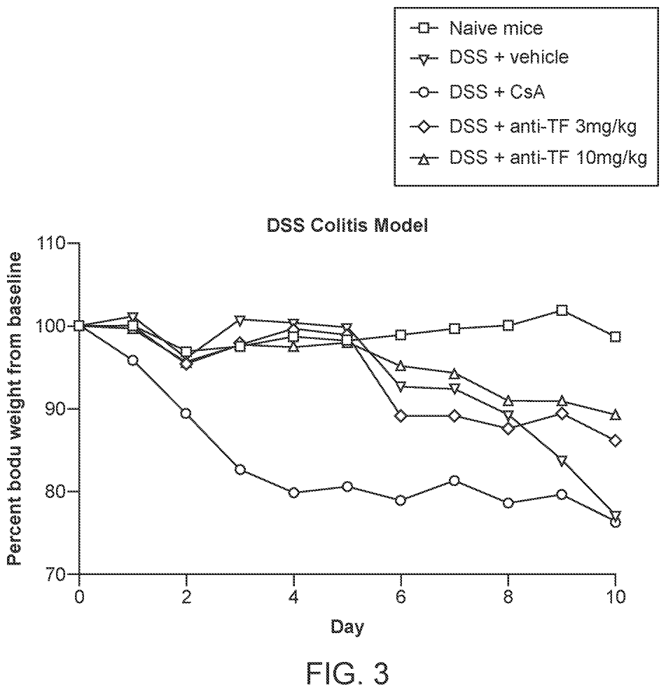

The results showed significant and early weight loss (˜20% weight loss relative to baseline) by Day 4 for the group 3 animals treated with CsA. Weight loss was comparable for the vehicle control group (group 2) and groups 4 and 5 for the first 5 days. Then, between Day 5 and Day 10, the vehicle control animals lost significantly more weight than the animals in groups 4 and 5. The results indicate that treatment with anti-TF antibody, 43D8, results in less weight loss relative to baseline levels than a comparator drug. They also indicate that treatment with anti-TF antibody results in less weight loss than would be experienced in the absence of the treatment (FIG. 3).

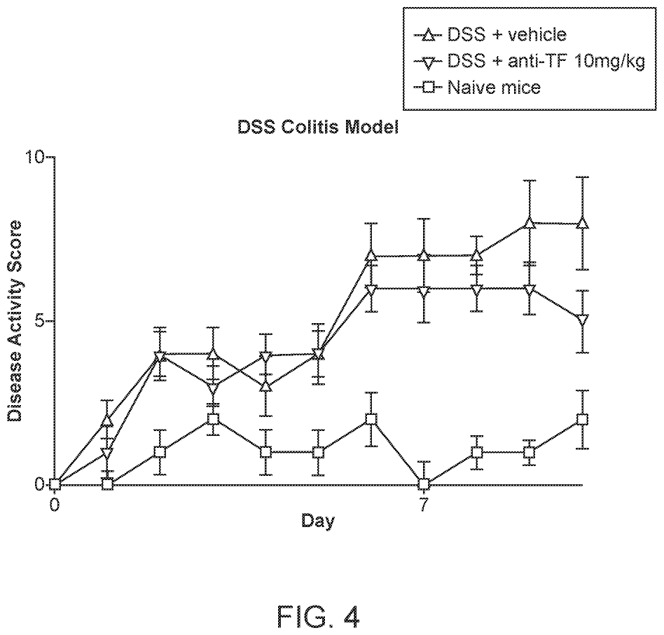

Disease activity was also analyzed using the above described metrics. The animals in group 5 (receiving 10 mg/kg of 43D8) had a lower (closer to normal) disease activity score than animals in the vehicle control (FIG. 4). There was no effect observed on disease activity in the vehicle control group or the group that received 3 mg/kg of 43D8. Overall, these results indicated that treatment with anti-TF antibody results in more normal stool consistency, less detectable blood and less weight loss than would be observed in the absence of the treatment.

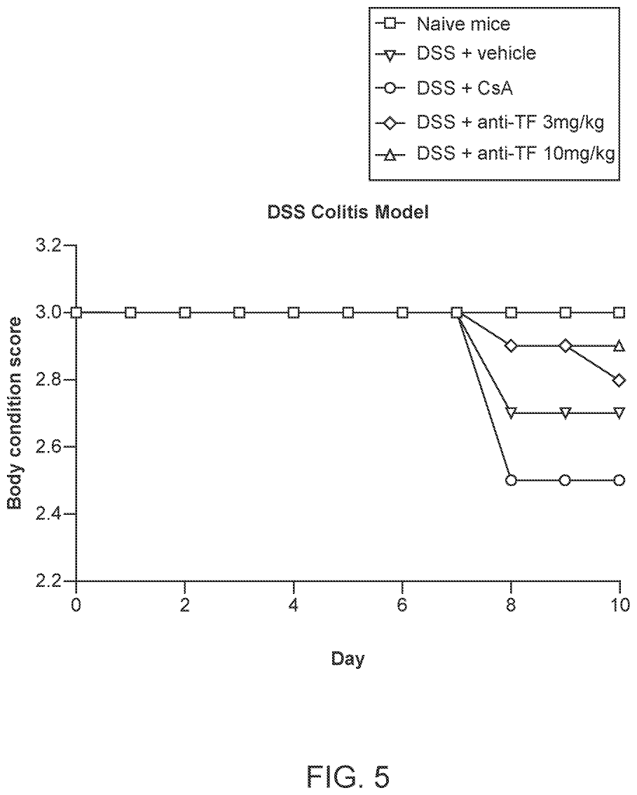

The results of the body conditioning revealed no change in the body condition of the mice in the study until Day 7, after which the group 2 CsA mice experienced the most significant deterioration in body condition. Only the naive group maintained a body condition of 3 (normal, well-conditioned state) throughout the study. Group 5 experienced the lowest reduction in body condition score, followed by group 4 (FIG. 5). The results indicate that treatment with anti-TF antibody improves body condition relative to a comparator treatment and relative to the body condition that would result from no treatment.

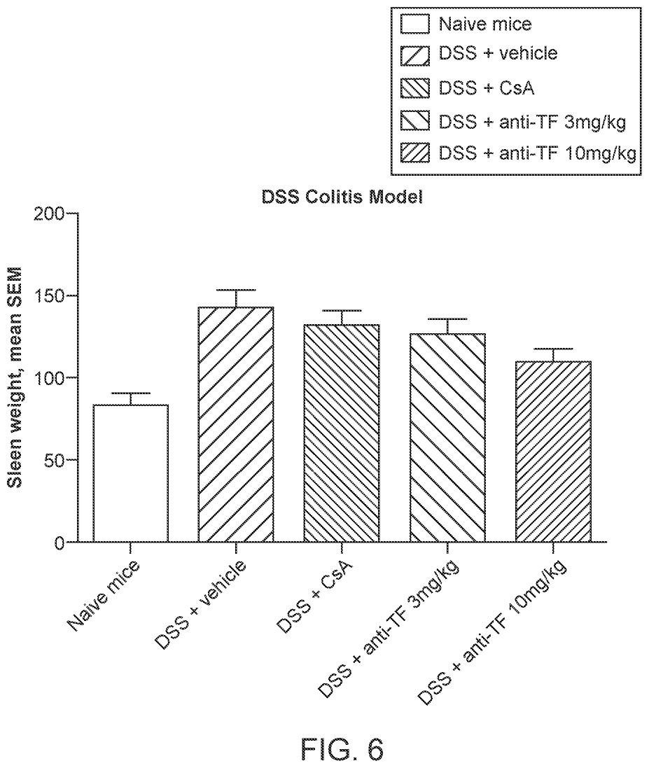

The results from measuring spleen weight showed a dose-dependent reduction in spleen weight in the 43D8 treatment groups relative to the vehicle control (FIG. 6). Those results suggest that treatment with anti-TF antibody can reverse or reduce the spleen enlargement that is often seen with inflammatory disease. The results are also indicative of a systemic anti-inflammatory effect from anti-TF antibody.

### Example 3: Influence of Anti-TF Antibody in a DSS-Colitis Model

Another in vivo study was conducted to determine the effects of an anti-TF antibody, (e.g., 43D8) on inflammatory endpoints in a colitis model. The study methods were the same as those outlined in Example 2, however, the concentration of DSS used to induce colitis and terminal day for study and controls were altered.

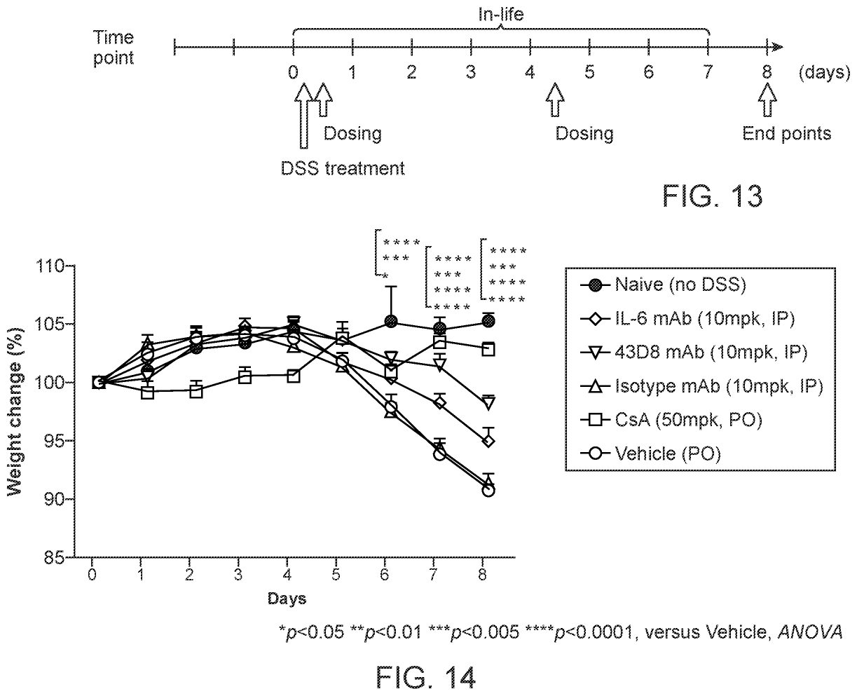

Briefly, on study Day 0, 8-12-week old male C57BL/6 mice received either sterile water ad libitum (Group 1, n=5) or 3% DSS dissolved into sterile water ad libitum (Groups 2-5, n=10 mice per group). On Day 0 and on day 4 mice from Groups 4, 5 and 6 received two doses of the Isotype, 43D8 mAb or anti-mouse 11-6 mAb. Also starting on Day 0 to Day 10, mice in Group 2 and 3 were treated once daily by oral gavage with the vehicle or positive control cyclosporine (CsA) at 80 mg/kg (Neoral, n=10). On Day 8, all animals were euthanized. The experimental design is shown in Table 67 and the time points and schedule are shown in FIG. 13. The study endpoints were body weight, DAI score, colon density (width/length), spleen weight, and histopathology.

TABLE 67
Experimental design for DSS model

| | | | Treatment | Systematic dosing | |
|---|---|---|---|---|---|
| Group | N | Test articles | (mg/kg) | Route | Frequency |
| 1 | 5 | Naïve (no DSS) | N/A | N/A | NA |
| 2 | 10 | Vehicle | N/A | PO | Once daily (QD) |
| 3 | 10 | CsA | 50 | PO | Once daily (QD) |
| 4 | 10 | Isotype antibody | 10 | IP | Twice (Day 0 and |
| | | | | | Day4) |
| 5 | 10 | 43D8 mAb | 10 | IP | Twice (Day 0 and |
| | | | | | Day 4) |
| 6 | 10 | IL-6 mAb | 10 | IP | Twice (Day 0 and |
| | | | | | Day 4) |

*IP = intraperitoneal; PO = oral administration

The results of body weight measurements, DAI score, colon density (ratio of colon weight/length), and spleen weight measurements are shown in FIGS. 14, 15, 16, and 17, respectively.

The results showed a delay in body weight loss by day 5 and at later times in the Group 5 mice (treated with 43D8 mAb), relative to the vehicle and isotype controls and the anti-IL-6 mAb mice. The delay in weight loss was highly significant relative to the vehicle control mice by day 6 (FIG. 14).

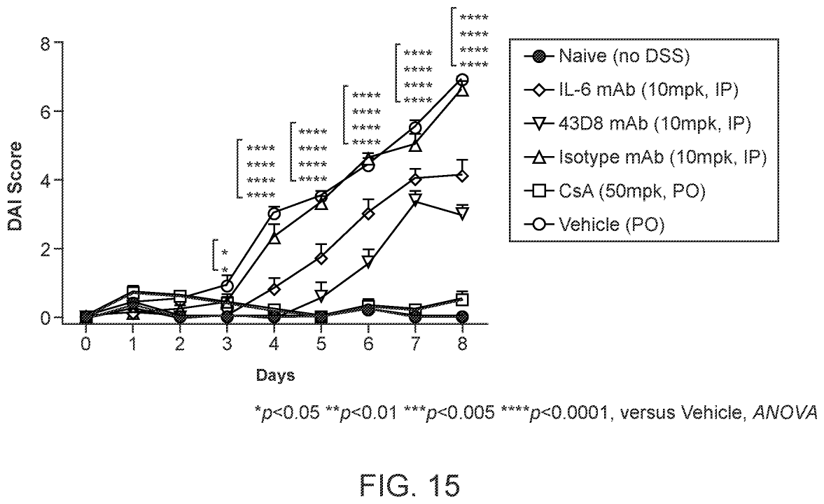

The results showed a significant improvement in the DAI score relative to the vehicle control mice by day 3. The DAI score was also lower in group 5 mice relative to group 6 mice (anti-IL-6 mAB) by day 4 (FIG. 15).

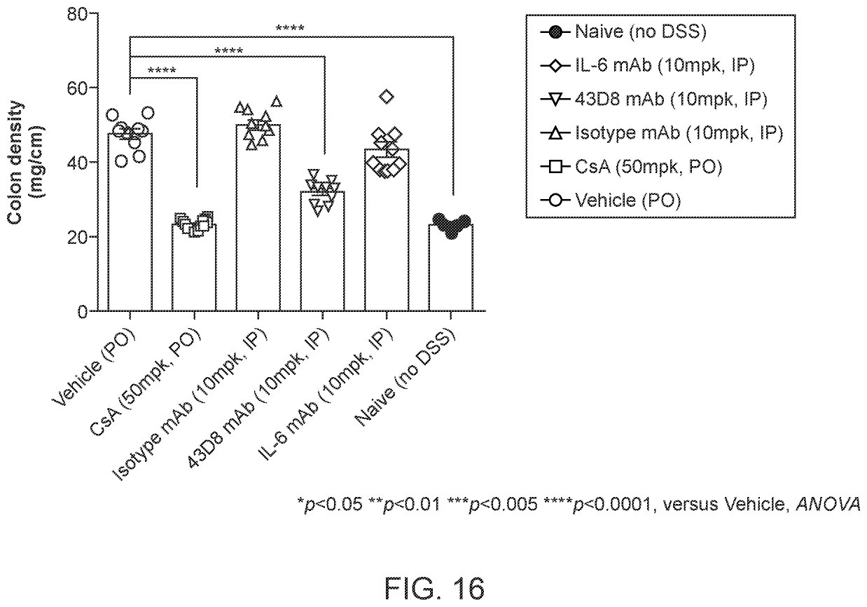

The results showed a significant improvement in the colon density of the group 5 mice relative to the vehicle control mice. The group 5 mice also exhibited lower colon density than the group 6 mice by the end of the study (FIG. 16).

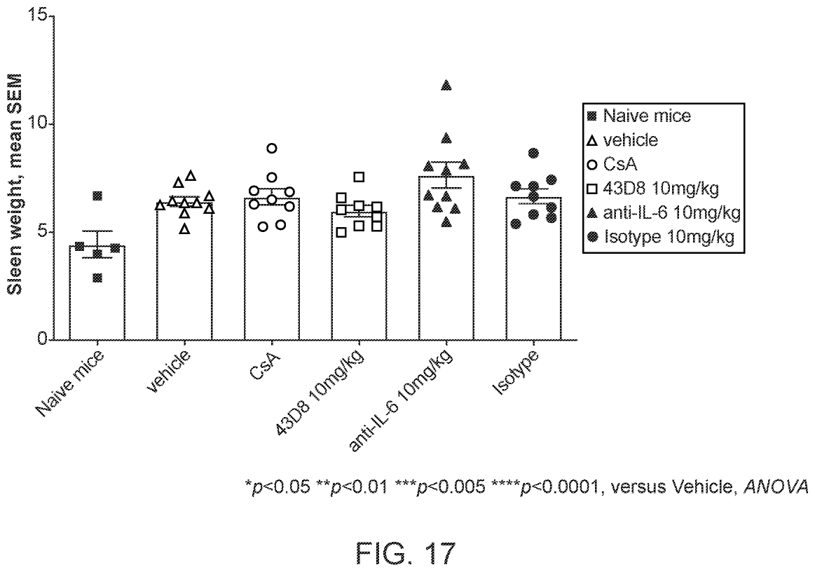

No significant differences in spleen weight were observed between the groups at the end of the study (FIG. 17).

### Example 4: Influence of Anti-TF Antibody in a TNBS-Colitis Model

An in vivo study was conducted to determine the effects of an anti-TF antibody, (e.g., 43D8) on inflammatory endpoints in a TNBS-colitis model.

In this colitis model, the administration of 2,4,6-trinitrobenzene sulfonic acid (TNBS) causes colitis-like pathologies. In general, the TNBS model is characterized by more focal damage in the colon than the DSS colitis model. It results in transmural colitis mainly driven by a TH1-mediated immune response and characterized by infiltration of the lamina propria with CD4b T cells, neutrophils, and macrophages. Anti-IFNg, anti-IL-12p40 have shown effect treatment in TNBS models.

Methods for making a TNBS-induced colitis model are known to those of ordinary skill in the art. See, for example, Antoniou, Efstathios, et al. Annals of medicine and surgery 11 (2016): 9-15, the relevant disclosures of which are herein incorporated by reference.

The effect of anti-TF was evaluated in a TNBS colitis model in which animals received an intracolonic injection of 2% TNBS to induce colitis (n=10 mice/group). Clinical observations, body weights, and DAI scoring were performed daily. Animals were treated with anti-TF antibody (e.g., 43D8), vehicle control or isotype control. An additional group received mesalamine as a positive control. The administration of anti-TF antibody (e.g., 43D8) showed no effect relative to the control. It is possible that the administration of anti-TF antibody in the TNBS model did not result in any effects because the TNBS model is a T cell dominated model. TF is known in the art to be expressed on activated myeloid cells, but not on T cells.

### Example 5: Influence of Anti-TF Antibody in Acute Lung Injury Model

An in vivo study was conducted to evaluate the effects of an anti-TF antibody, (e.g., 43D8), on inflammatory endpoints in a lipopolysaccharide (LPS)-induced acute lung injury model. Acute lung injury (ALI) and its most severe manifestation, the acute respiratory distress syndrome (ARDS), is a clinical syndrome defined by acute hypoxemic respiratory failure, bilateral pulmonary infiltrates consistent with edema, and normal cardiac filling pressures.

For this study, 48 male BALB/c mice were randomly and prospectively assigned to five groups: a group of six (n=6), a group of twelve (n=12), and three groups of ten (n=10 per group) animals each. On Day 0, animals in Groups 2-5 were dosed according to Table 63, 60 minutes prior to LPS administration. Dexamethasone (3 mg/kg) was again administered on Day 1 (24 hours post LPS) to the Group 3 animals (positive control). All animals were anesthetized using isoflurane and once each animal was non-responsive to toe pinch, the animal was challenged with an intranasal administration of 10 μg of LPS intranasally (IN) in 25 μl (Groups 2-5 only) or saline as a control (Group 1). Animals were then released into a recovery cage until they woke up.

TABLE 63
Experimental Design for ALI Study. TA denotes test article (43D8 antibody)

| | | LPS (IN) | | | | | |
|---|---|---|---|---|---|---|---|
| | No | Day 0 | | | | Dose | Endpoint/Terminal |
| Group | Animals | (0 h) | Treatment | Dose | Route | Schedule | Collections (48 h) |
| 1 | 6 | Saline | — | — | — | — | 1. Blood (plasma) |
| 2 | 12 | 10 μg | Vehicle | — | IP | −1 hr | 2. Lungs (sample |
| 3 | 10 | | Dexamethasone | 3 mg/kg | IP | −1 hr, 24 hr | collection) |
| 4 | 10 | | TA #1 | 1 mg/kg | IP | −1 hr | BAL Fluid: |
| 5 | 10 | | | 10 mg/kg | IP | −1 hr | Differential |
| | | | | | | | Counts, Total |
| | | | | | | | Protein |
| | | | | | | | Lung Histology: |
| | | | | | | | H&E staining |
| | | | | | | | Lung Tissue: snap |
| | | | | | | | frozen |

All animals were weighed and evaluated for respiratory distress daily (defined as an increase in respiratory rate and/or obvious respiratory effort). Animals with severe respiratory distress, or animals that lost greater than 20% of their total starting body weight, were euthanized within 2 hours of observation.

At 48 hours post-LPS challenge all animals were sacrificed with an overdose of xylazine and bronco-alveolar lavage (BAL) of the right lung only was performed (by tying off the left lung) for total and differential cell counts as well as total protein quantification and cytokine quantitation by Luminex. Lungs were then weighed (total lung weight and right lung weight). The right lung was frozen in liquid nitrogen and stored at −80° C. The left lobe of the lung was insufflated with 10% NBF, fixed in 10% NBF for 24 hours and then switched to PBS, and subsequently processed for histology. The formalin-fixed lung was embedded in paraffin, sectioned at 5 microns, and slides stained with hematoxylin and eosin (H&E). All slides were evaluated by a board-certified veterinary pathologist who used a scoring system to evaluate extent of lung injury and inflammation. Table 64 and Table 65 show the scoring system for leukocyte infiltration.

TABLE 64
Histopathological scoring for interstitial or
alveolar neutrophil infiltration in ALI model

| Score | Interstitial or Alveolar Neutrophil Infiltration |
|---|---|
| 0 | not present |
| 1 | minimal (<10% of sample affected) |
| 2 | mild (10-25% of the sample affected) |
| 3 | moderate (26-50% of the sample affected) |
| 4 | marked (51-75% of the sample affected) |
| 5 | severe (>75% of the sample affected) |

TABLE 65
Histopathological scoring for mononuclear cell infiltration/aggregate
formation in perivascular/peribronchiolar zones

| Score | Interstitial or Alveolar Neutrophil Infiltration |
|---|---|
| 0 | not present |
| 1 | minimal (focal infiltrate or scattered infiltrating cells) |
| 2 | mild (multifocal infiltrate or small aggregate formation) |
| 3 | moderate (multifocal aggregate formation) |
| 4 | marked (most blood vessels/bronchioles are surrounded by |
| | aggregates) |
| 5 | severe (all blood vessels/bronchioles surrounded by large |
| | and coalescing aggregates) |

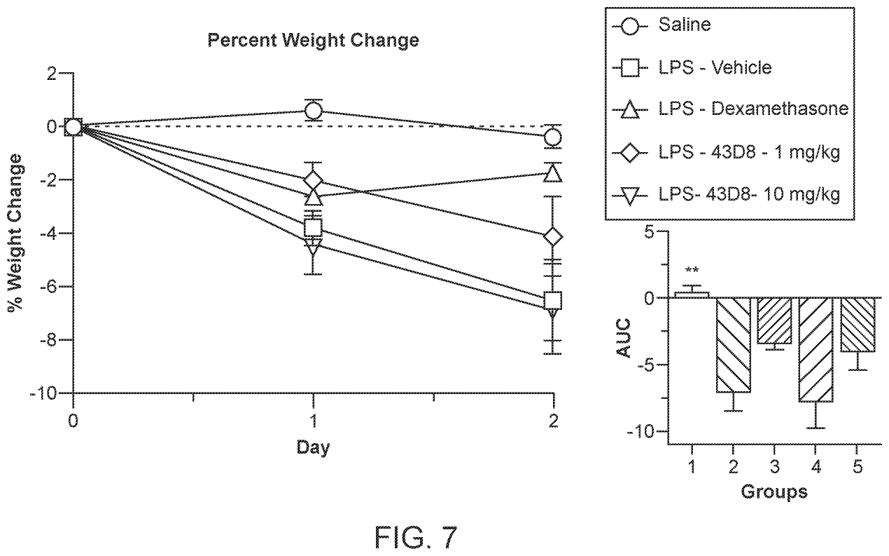

The body weight results showed the highest weight loss in group receiving 1 mg/kg 431D8. The vehicle control group and group 4 (1 mg/kg 431D8) had comparable percent weight loss by the end of the study (˜6% weight loss relative to baseline). In contrast, the positive control group (dexamethasone) only exhibited about 2% weight loss relative to baseline at the end of the study. Group 5 (receiving 10 mg/kg) exhibited less weight loss than the vehicle control, but more weight loss than the positive control (FIG. 7). The results indicate that in an ALI subject, treatment with anti-TF antibody (431D8) can result in less weight loss than would be experienced in the absence of treatment (a protective effect on body weight loss). The results also indicate that anti-TF antibody counters weight loss in a dose-dependent manner.

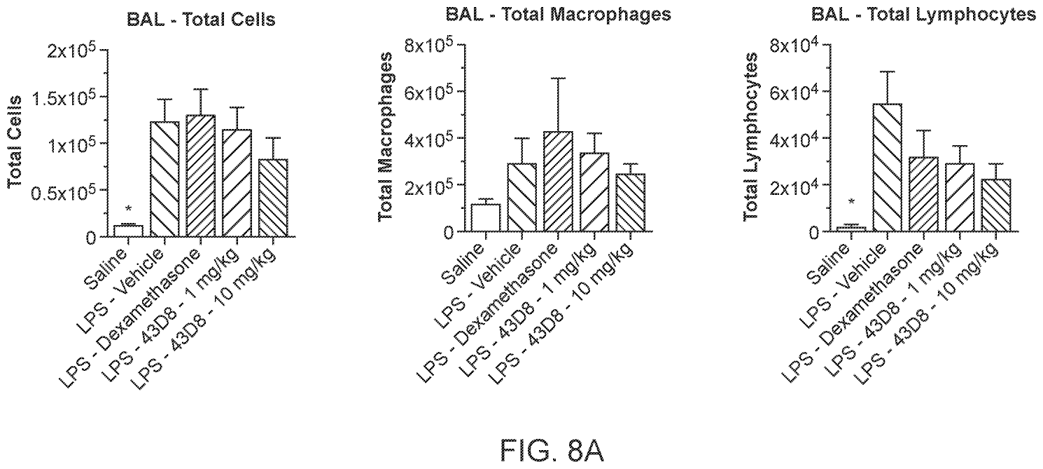

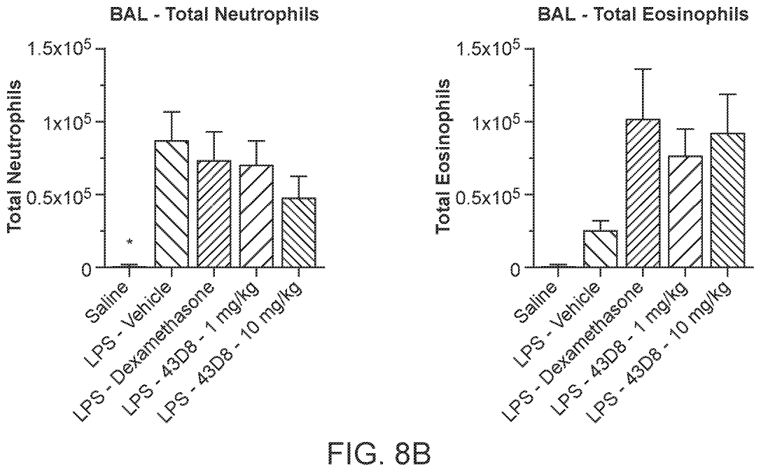

The results from the BAL cell differential counts revealed that treatment with anti-TF antibody (43D8) resulted in a lower total leukocyte count than the positive control and vehicle control. The total macrophage count in group 4 (1 mg/kg 43D8) was not significantly lower than the vehicle control, however, the total macrophage count for group 5 (10 mg/kg 43D8) was lower than the vehicle control (as well as the positive control). The total lymphocyte count and total neutrophil count for groups 4 and 5 were lower than their respective vehicle controls and the reduction in their counts was dose dependent. In contrast, the total eosinophil counts for groups 4 and 5 were significantly higher than the vehicle control. Overall, the results revealed a decrease in lymphocyte, macrophage and neutrophil infiltrate in BAL fluid in groups treated with 43D8 and the decreases were comparable or better than the positive control (dexamethasone) (FIGS. 8A and 8B).

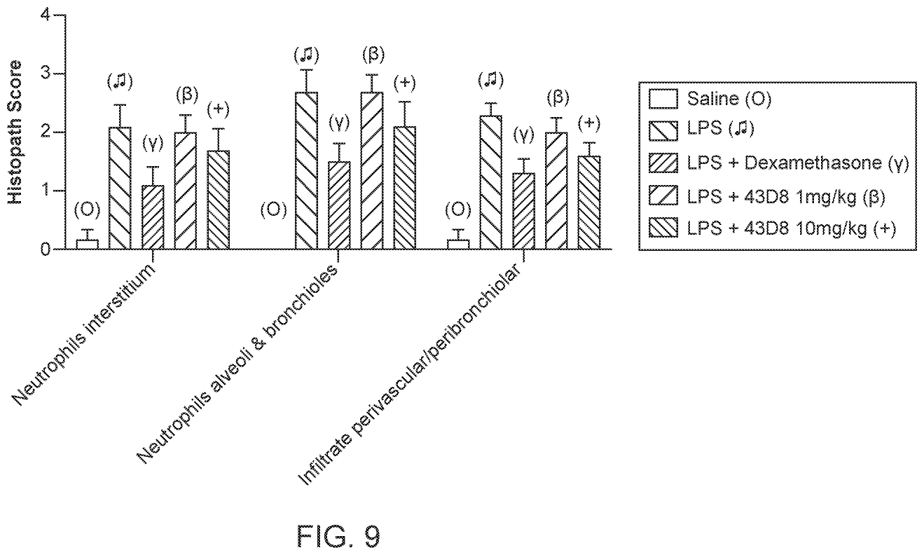

For the histopathological analysis, group 5 (10 mg/kg of 43D8) exhibited a slight decrease in neutrophil infiltration into the interstitium, alveoli, and bronchioles and mononuclear cell infiltration into perivascular/peribronchiolar tissue relative to the vehicle control. The differences between group 4 (1 mg/kg of 43D8) and the vehicle control in neutrophil infiltration into the interstitium, alveoli, and bronchioles were not significant. None of the test article groups were as effective as the positive control (dexamethasone) in reducing neutrophil infiltration into the interstitium, alveoli, and bronchioles and mononuclear cell infiltration into perivascular/peribronchiolar tissue (FIG. 9).

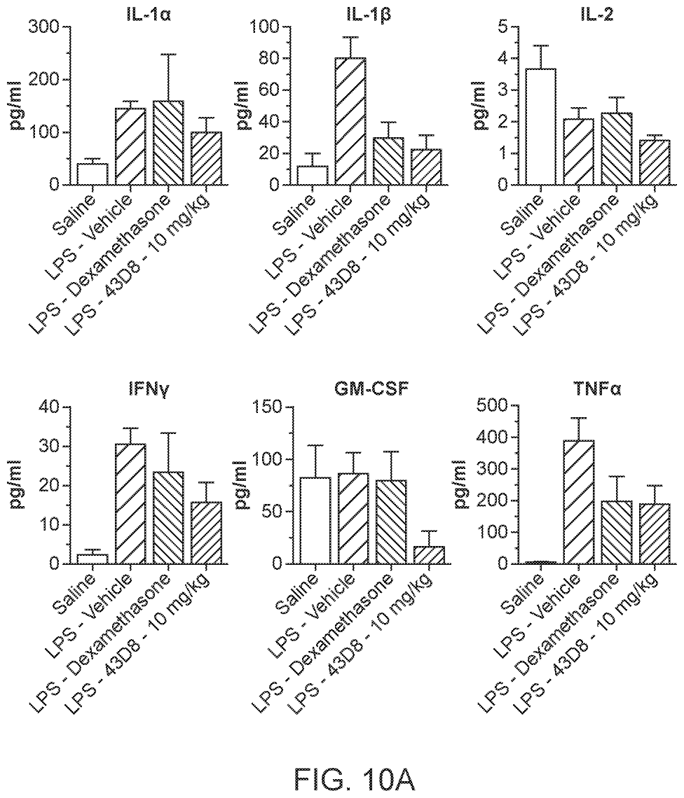

The results for inflammatory cytokines are shown in FIGS. 10A and 10B. The 10 mg/kg 43D8 group exhibited a significant reduction in cytokine concentration relative to the vehicle control. In all cases, except for IL-6 and TNFα, the 10 mg/kg 43D8 group exhibited a significant reduction in inflammatory cytokine levels relative to the positive control (dexamethasone). These results are also indicative of a reduction in local inflammation as a result of treatment with anti-TF antibody.

### Example 6: Influence of Anti-TF Antibody in RSV Model

An in vivo study was conducted to evaluate the effects of an anti-TF antibody, (e.g., 43D8), on BAL differential cell counts in an respiratory syncytial virus (RSV) model.

Female BALB/c mice of ˜6-8 weeks of age at study initiation were administered 50 μL of 8.5×10⁵ titer RSV-A2 stock which originally was acquired from ATCC (VR-1540) by intranasal inoculation. Group 1 received Hep-2 supernatant as a mock control. All inoculations were performed while the animals were under the influence of an inhalant anesthesia.

At 2 hours post-RSV-A2 infection, the molecules were administered in volumes formulated to deliver the amounts in Table 66 via an intravenous (IV) or oral (PO) route (n=10 per group). AZD1480 (a JAK inhibitor served as a positive control). At 5 days post infection, lungs were harvested from each animal and weighed. The lungs were then flushed with Hanks Buffer and Bronchoalveolar Lavage Fluid (BALF), harvested from each animal, and total BAL leukocyte counted. BALF was split into 3 aliquots and stored at −80° C. The right lung and left lung was bisected, weighed, individually snap frozen and stored at −80° C. The right lung was stored for viral quantification.

TABLE 66
Experimental design for the RSV model study

| | | | Route, Schedule | Dose | |
|---|---|---|---|---|---|
| Group | RSV A | Treatment | (t + 2 hours) | (mg/kg) | |
| 1 | HEP-2 lysate | none | none | NA | |
| 2 | 8.5 × 10⁵ pfu | Vehicle | IV, single | — | |
| | Intranasal | (PBS) | Day 1 | | |
| 3 | inoculation | 43D8 | IV, single | 1 | mg/kg |
| | | | Day 1 | | |
| 4 | | 43D8 | IV, single | 10 | mg/kg |
| | | | Day 1 | | |
| 5 | | AZD1480 | PO, BID | 30 | mg/kg |
| | | | from day | | |
| | | | 1 to day 4 | | |

Slides were prepared from remaining BAL leukocytes, fixed and stained with May Geimsa stain and differential counts were recorded manually. An aliquot BAL fluid was evaluated using a mouse cytokine panel from Meso Scale Discovery (MSD, Rockville, Maryland).

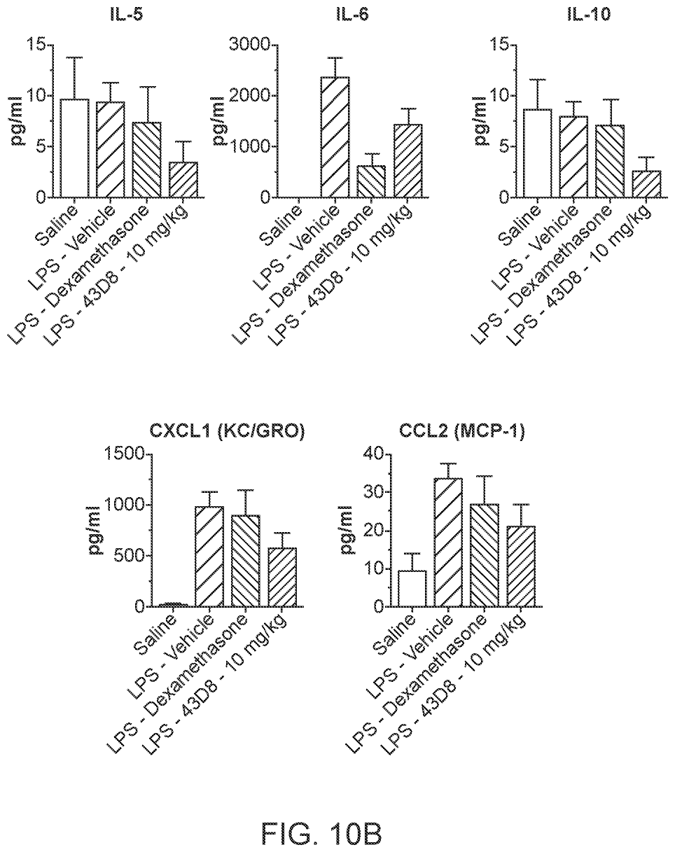

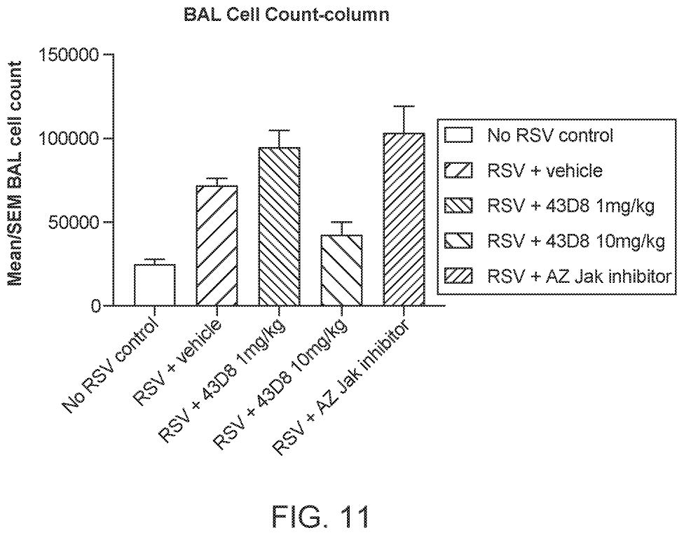

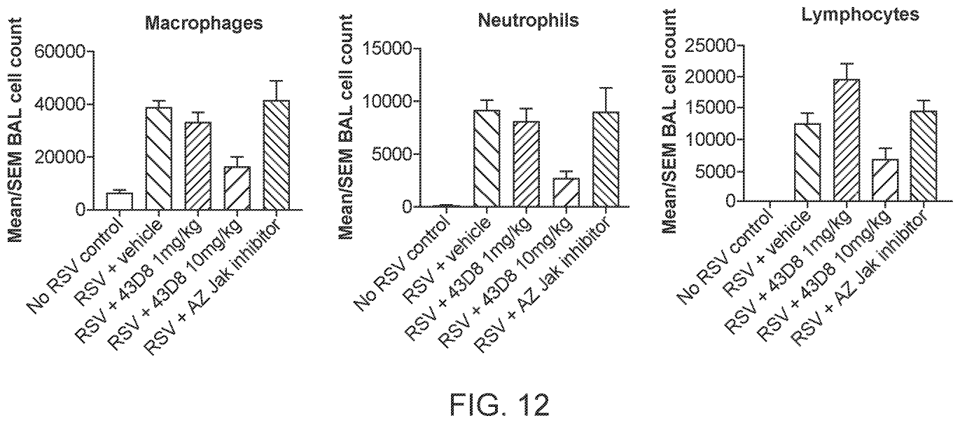

The results showed a significant reduction in the mean leukocyte count for group 4 (10 mg/kg of 43D8) relative to the vehicle control and relative to the positive control (AZD1480) (FIG. 11). As shown in FIG. 12, group 4 exhibited a significant decrease in mean macrophage BAL count, mean neutrophil BAL count, and mean lymphocyte BAL count. The results also reveal a dose-dependent response to treatment with anti-TF antibody. There were no changes in monocyte and eosinophil counts observed (data not shown). Overall, these results are consistent with TF mediating chemotaxis.

### Example 7: Influence of Anti-TF Antibody in Poly I:C Model

An in vivo study was conducted to evaluate the effects of an anti-TF antibody, (e.g., 43D8) on inflammatory endpoints in a polyinosine-polycytidylic acid (Poly(I:C)) model. The poly I:C model mimics the in vivo responses of the lung to viral infection. In the model, mice are administered Poly C, which is a synthetic analogue ofdouble-stranded (ds)RNA and is a TL3 ligand. It is often used in vivo to study viral recognition by host cell innate immune system and subsequent cytokine storm and inflammation.

Briefly, on Days 1, 2, and 3, all mice were anesthetized by isoflurane inhalation. Mice were held in an upright position and 50 fL Poly (C) in PBS was administered into the animal's nares using a pipette. On Day 2, at 3 hrs after the second intra-nasal challenge, 10 mice from selected group were terminally anaesthetized and, blood collection and three consecutive bronchoalveolar lavage (BAL) collections were performed. On Day 4, at 24 hrs after the last intra-nasal challenge, remaining mice were terminally anaesthetized and, blood collection and three consecutive broncheoalveolar lavage (BAL) collections were performed. BAL measurements were assessed by multiplex electrochemiluminescence MSD assay.

Dosing with test items: vehicle, isotype control and the antibody (e.g., 43D8) was injected intra-peritoneally (IP) on Day 1 at 2 hrs prior to poly:SC injection.

The doses and groups are provided in Tables 68 and 69.

TABLE 68
Study design examples for groups 1-4

| | | Poly:IC Dose | TI dose level, | | | |
|---|---|---|---|---|---|---|
| | | level, route, | route, volume, | | | |
| | | dosing | dosing | | Necropsy | Necropsy |
| Gr | Treatment | frequency | frequency | N | 1 | 2 |
| 1 | Naïve/ | — | 0 mg/kg, IP, | 10 | — | Day 4, |
| | Vehicle | | once on Day 1 | | | 24 hrs |
| | (PBS) | | | | | post last |
| | | | | | | poly:IC |
| 2 | Poly:(IC)/ | 50 μg/50 μL, | 0 mg/kg, IP, | 10 + 10 | Day 2, | Day 4, |
| | Vehicle | intranasal, | 10 mL/kg, once | | 3 hrs post | 24 hrs |
| | (PBS) | Days 1, 2, 3 | on Day 1 @2 hrs | | 2^{nd} poly:IC | post last |
| | | | prior to Poly:IC | | | poly:IC |
| 3 | Poly (I:C)/ | 50 μg/50 μL, | 0 mg/kg, IP, | 10 + 10 | Day 2, | Day 4, |
| | antibody | intranasal, | 10 mL/kg, once | | 3 hrs post | 24 hrs |
| | | Days 1, 2, 3 | on Day 1 @2 hrs | | 2^{nd} poly:IC | post last |
| | | | prior to Poly:IC | | | poly:IC |
| 4 | Poly (I:C)/ | 50 μg/50 μL, | 0 mg/kg, IP, | 10 | — | Day 4, |
| | Isotype | intranasal, | 10 mL/kg, once | | | 24 hrs |
| | control | Days 1, 2, 3 | on Day 1 @2 hrs | | | post last |
| | antivody | | prior to Poly:IC | | | poly:IC |

TABLE 69
Doses of 43D8 mg of antibody/kg

| | | Dose | |
|---|---|---|---|
| Group | Treatment and Test Article | (mg/kg) | Harvest time |
| 1 | Vehicle (naïve mice) | NA | Day 3 |
| 2 | Vehicle + Poly I:C | NA | Day 2 |
| 3 | 43D8 + Poly I:C | 10 mg/kg | Day 2 |
| 5 | Vehicle + Poly I:C | NA | Day 3 |
| 6 | Isotype + Poly I:C | 10 mg/kg | Day 3 |
| 7 | 43D8 + Poly I:C | 10 mg/kg | Day 3 |

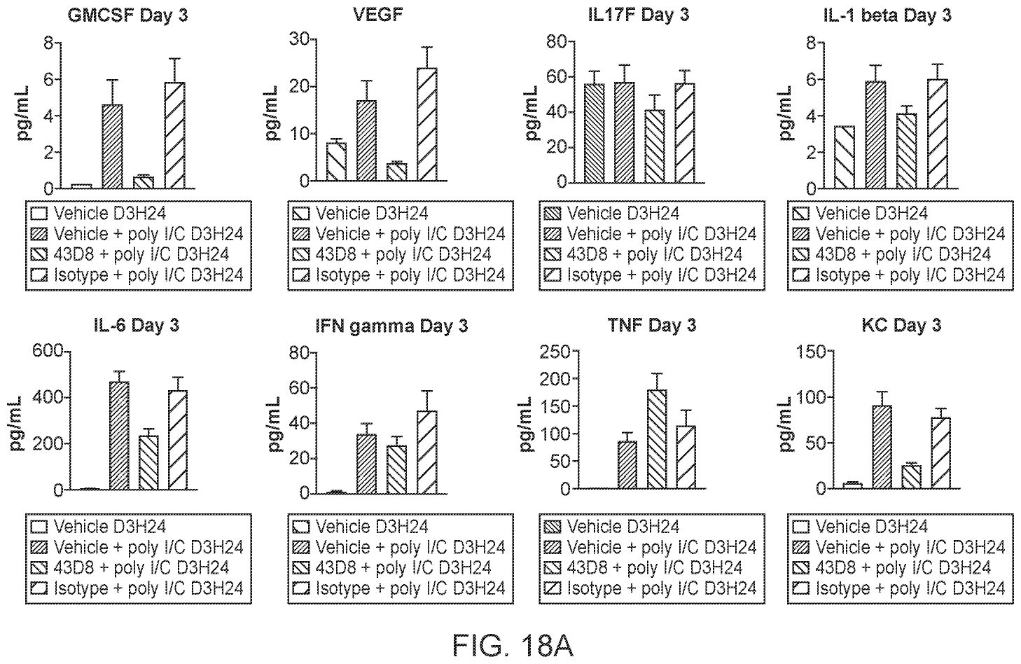

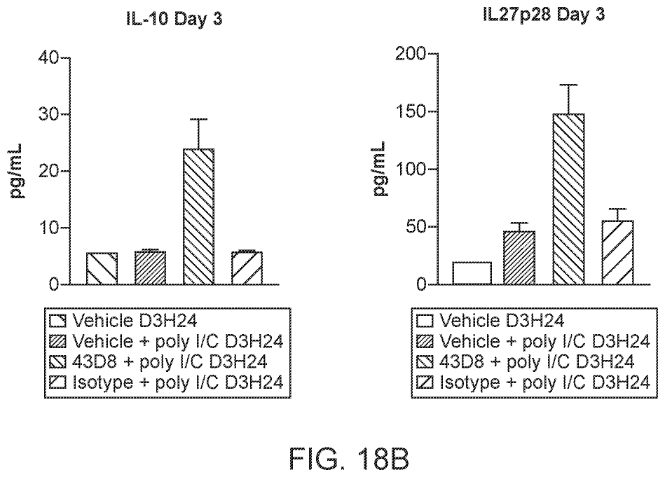

FIG. 18A shows proinflammatory cytokine levels from Day 3 of the study. The results showed a notable reduction in the levels of GMCSF, VEGF, IL17F, IL-1 beta, IL-6, IFN gamma and KC proinflammatory markers on Day 3 in Group 7 (43D8+Poly I:C treatment group) relative to the Group 5 (vehicle+Poly I:C control) and group 7 (isotype+Poly I:C control).

FIG. 18B shows the levels of anti-inflammatory markers IL-10 and IL28p28 from Day 3 of the study. Both markers were substantially increased on Day 3 in Group 7 (43D8+Poly I:C treatment group) relative to the Group 5 (vehicle+Poly I:C control) and group 7 (isotype+Poly I:C control).

In comparison, the magnitude of response was smaller at Day 2.

### Example 8: Influence of Anti-TF Antibody in COVID Model

An in vivo study was conducted to evaluate the effects of an anti-TF antibody, (e.g., 43D8) in a COVID model. This model was used to evaluate the treatment effects of anti-TF mAb 43D8 in the plasma and on the lungs of 4-8 week-old B6.Cg-Tg(K18-ACE2) mice thatexoress human ACE2 (The Jackson Laboratory), following a SARS-CoV-2 intranasal challenge.

Briefly, mice in groups 1 through 4 were challenged with neat stock of SARS-CoV-2 on Study Day 1 by intranasal inoculation according to Table 70. Mice in groups 1 through 4 received a single dose of test or control article approximately 2 hours (15 minutes) prior to challenge. Mice in groups 1 and 2 were euthanized for sample collection on Study Day 4. Mice in groups 3 and 4 surviving on Study Day 8 were euthanized for sample collection. Mice were observed, with observations recorded, a minimum of twice daily, at least six hours apart for the duration of the study period, except on the day of humane termination when only one observation was conducted. Body weights were collected pre-study and daily during study.

TABLE 70
Experimental details for the anti-TF-COVID model study

| | | | | | | Scheduled |
|---|---|---|---|---|---|---|
| | Number | Challenge | | Treatment | Treatment | Termination |
| Group | of Mice | Dose/Route | Treatment | Dose/Route | Initiation | (Study Day) |
| 1 | 5 M & 5F | SARS-CoV-2/ | Saline | 10 mg/kg/ | ~2 hours | 4 |
| 2 | 5 M & 5F | 1E+03/IN¹ | 43D8 | IP² | (±15 | 4 |
| 3 | 5 M & 5F | | Saline | | minutes) | 8 |
| 4 | 5 M & 5F | | 43D8 | | prior to | 8 |
| | | | | | change | |

F = Female
IN = Intranasal
IP = Intraperitoneal
M = Male
¹12.5 μL will be instilled into the right and left nares for a total volume of 25 μL.
²Treatments will be delivered at a target volume of 10 mL/kg.

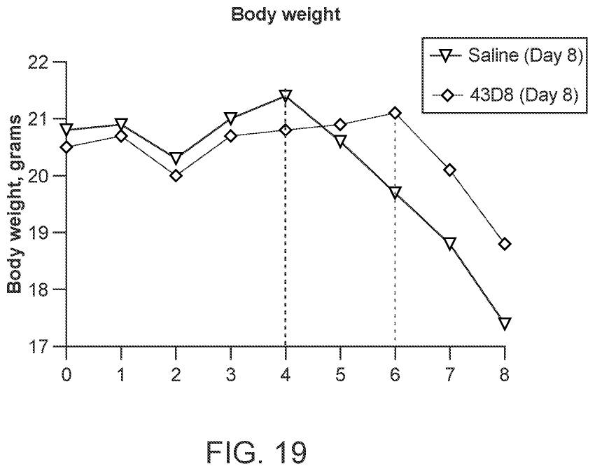

FIG. 19 shows the results for body weight measurements over the course of the study. Table 71 shows the results for the clinical observations in the saline and 43D8 treatment group.

TABLE 71
Clinical observations in COVID model

| | Clinical observations | | Clinical observations |
|---|---|---|---|
| Saline | for Saline | 43D8 | for 43D8 |
| 2254 | Hunched, rough coat | 2268 | Normal through end of study |
| 2255 | Died on Day 7 | 2269 | Normal through end of study |
| 2266 | Normal through end of | 2271 | Normal through end of study |
| | study | | |
| 2267 | Hunched, lethargic, | 2274 | Normal through end of study |
| | labored breathing | | |
| 2270 | Died on Day 8 | 2275 | Normal through end of study |
| 2281 | Normal through end of | 2277 | Lethargic, labored breathing |
| | study | | |
| 2286 | Normal through end of | 2279 | Normal through end of study |
| | study | | |
| 2288 | Hunched, labored | 2280 | Normal through end of study |
| | breathing | | |
| 2297 | Normal through end of | 2283 | Normal through end of study |
| | study | | |
| 2298 | Hunched, lethargic, | 2300 | Normal through end of study |
| | decreased breathing | | |

Overall, the results showed a delay in weight loss for the 431D8 treatment group. No deaths were observed in the 431D8 treatment group, while 2 animals died on study in control group. Most animals in control group had significant clinical observations, while only 1 animal showed signs of disease in 431D8 treatment group.

Lung Histopathology

To evaluate the effect of anti-TF antibodies (e.g., 431D8) on the lung histopathology, animals are euthanized at the end of the study. Following euthanasia, tissue samples from the lungs are placed in 10% neutral-buffered formalin (NBF) for >48 hours then transferred to 70% ethanol for >72 hours. The samples are embedded in paraffin, sectioned and stained with hematoxylin and eosin (H&E) for histopathological analysis.

Viral Titer Measurements

To evaluate the impact of the anti-TF antibodies (e.g., 43D8) on SARS-CoV-2 viral titer levels, Briefly, ˜4-5 mm³ samples are aseptically collected from the right lung after euthanasia and preserved in RNAlater. A quantitative real-time PCR (qRT-PCR) assay is used to measure viral load in samples. Nasal, pharyngeal and rectal samples are also analyzed using qRT-PCR at regular intervals over the course of the study. Methods for measuring and analyzing viral titer data are known to those of ordinary skill in the art. See, for example, Roberts, Anjeanette, et al. PLoS pathogens 3.1 (2007): e5, the relevant disclosures of which are herein incorporated by reference.

BAL Cytokine/Chemokine Measurements

To evaluate the effect of anti-TF antibodies (e.g., 43D8) on cytokine and chemokine levels, mice that are terminally anaesthetized during the study undergo blood collection and bronchoalveolar lavage (BAL) collections. Proinflammatory cytokines (e.g., GMCSF, VEGF, IL17F, IL-1 beta, IL-6, IFN gamma, TNF and KC) are measured. Anti-inflammatory cytokines (e.g., IL-10 and IL27p28 are measured).

D-Dimer Measurements

To evaluate the effect of anti-TF antibodies (e.g., 43D8) on D-dimer levels, blood is collected to obtain plasma and serum samples from the treatment (43D8) and control (saline) group. The plasma and serum samples are analyzed for D-dimers using ELISA. Examples of methods for measuring and analyzing d-dimer levels in a mouse model are provided in, for example, Weiler, Hartmut, et al. “Characterization of a mouse model for thrombomodulin deficiency.” Arteriosclerosis, thrombosis, and vascular biology 21.9 (2001): 1531-1537, the relevant disclosures of which are herein incorporated by reference.

### Example 9: Influence of Anti-TF Antibody on Myocardial Infarction (MI) Recovery

PAR2, expressed by macrophages, as well as the TF cytoplasmic domain have detrimental effects on postischemic recovery in myocardial infarction (MI) in mice. An in vivo study was conducted to evaluate the effects of an anti-TF antibody (e.g., 43D8) and TF signaling blockade on recovery from myocardial infarction (MI). Methods for making and testing end points in MI models are known to those of ordinary skill in the art. See, for example, Molitor, Michael, et al. Cardiovascular research 117.1 (2021): 162-177, the relevant disclosures of which are herein incorporated by reference.

Briefly, to induce MI, mice underwent a permanent ligation of the left anterior descent coronary artery. Cardiac function was monitored by high frequency ultrasound intravital imaging. After induction of MI, mice (8 mice/per group) received 10 mg of 43D8 antibody/kg or isotype control in the backbone. The administration of the antibody and control was at day 1 and day 4 after MI, and evaluation of cardiac function was conducted on day 7 by high frequency ultrasound intravital imaging. The high frequency ultrasound intravital imaging was used to determine the wall motion score index (infarct size), left ventricular ejection fraction, and left ventricular end diastolic volume. Mice were euthanized at day 7 and the ischemic heart tissues was evaluated for inflammatory cell recruitment in the infarcted myocardium. Inflammatory cell recruitment was analyzed using fluorescence-activated cell sorting (FACS) in infarcted myocardial tissue.

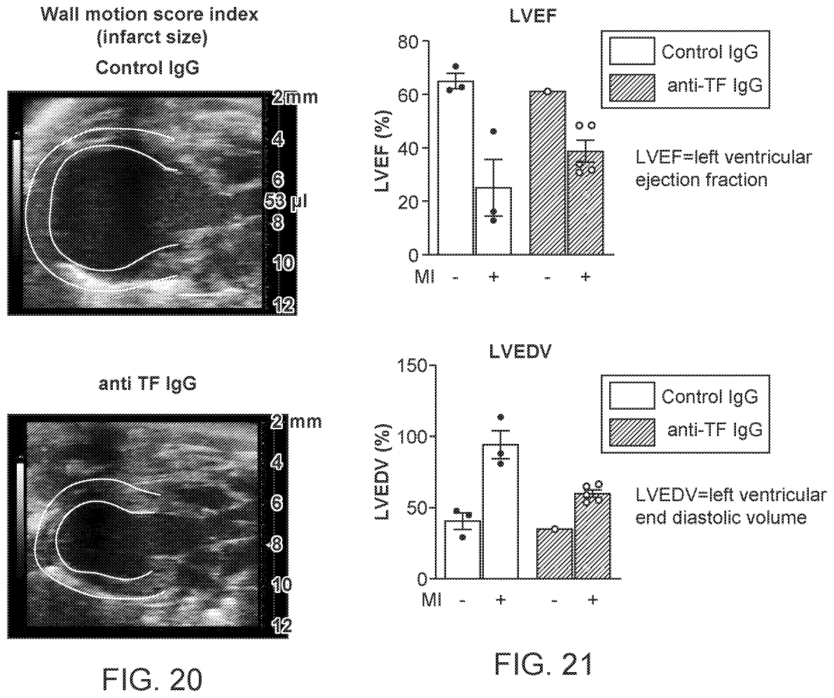

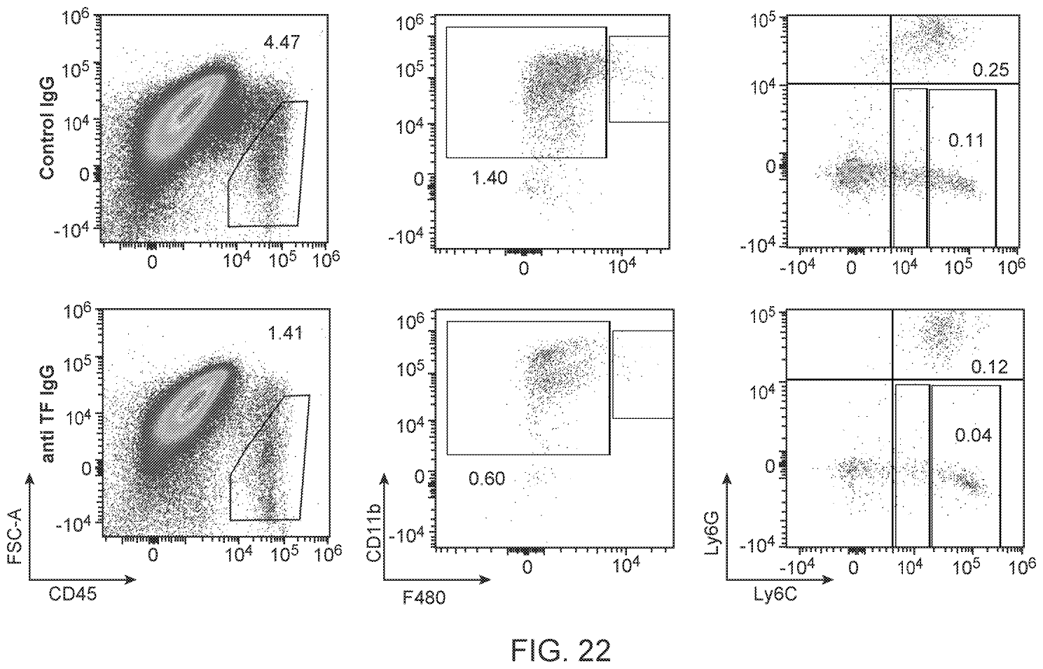

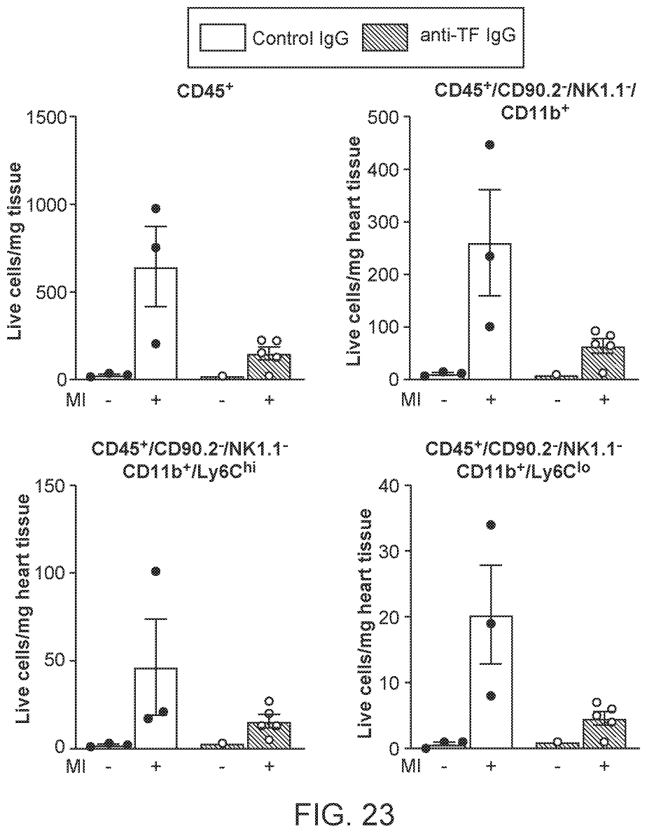

The results are shown in FIGS. 20-23. The results revealed that infarct size was reduced in the group treated with anti-TF antibody relative to the isotype control (FIG. 20). MI reduces left ventricular ejection fraction and the results showed that treatment with the anti-TF antibody restored left ventricular ejection fraction more than the isotype control. MI significantly increases left ventricular end diastolic volume, and the results revealed that treatment with the anti-TF antibody reduces the left ventricular end diastolic volume more than the isotype control (FIG. 21). The results also showed a reduction of inflammatory cell infiltration in infarcted myocardium (FIGS. 22 and 23).

### Cytokine Expression and PAR2 Signaling

The results above may be an indication that the anti-TF antibody interrupts TF-Par2 signalling. To evaluate the effect of the anti-TF antibody on TF-Par2 signalling, inflammatory cytokine expression is measured using RT-PCR and ERK1/2 phosphorylation was used as a marker for PAR2 signaling. The inflammatory end points are measured at day 7 and day 28.

### Example 10: Influence of Anti-TF Antibody in Collagen Antibody-Induced Arthritis (CAIA) Model

An in vivo study is conducted to evaluate the effects of an anti-TF antibody, (e.g., 43D8), on inflammatory endpoints in an CAIA model. In the CAIA model, arthritis is induced using monoclonal antibodies against type II collagen.

Briefly, mice of the same sex, ˜21 days of age at study initiation, are randomly and prospectively assigned to five groups (n=10 per group):
Group 1: naïve
Group 2: vehicle control (PBS)
Group 3: Test article (10 mg/kg of 43D8)
Group 4: positive control (dexamethasone)
Group 5: anti-TNFα

On Day 0, the disease is induced in groups 2-5 by administering an anti-Type II collagen antibody cocktail. On the same day, the animals in groups 2-5 receive the vehicle, positive controls or test article. On Day 3, the animals are administered LPS intraperitoneally (IP). Thereafter the animals are examined daily to assess changes in mobility that would be indicative of arthritis, weight measurements and body conditioning scoring as illustrated in (FIG. 2).

The animals are euthanized at the end of the study (Day 12). Following euthanasia, the animal are measured (length determined) and weighed. Weight/length ratio is calculated for each animal. The animals are dissected and the weight of the spleen is determined. Samples of the synovial fluid are collected and examined for mononuclear cell infiltration using IHC. Tissue samples from the site of induced arthritis are placed in 10% neutral-buffered formalin (NBF) for 24 hours, followed by 70% ethanol. The samples are embedded in paraffin, sectioned and stained with hematoxylin and eosin (H&E) for histopathological analysis. The bones at the site of induced arthritis are also observed for bone erosion. Additional endpoints measured in the animals include the clinical arthritis score, paw-pad thickness (e.g., where the arthritis is induced in a paw), and general clinical observation. (See, for example, MacKenzie J D et al. Radiology. 2011; 259(2):414-420 and Jung, E G., et al. BMC complementary and alternative medicine 15.1 (2015): 1-11., each of which is incorporated by reference in its entirety). The results show a significant improvement in measured metric(s) for anti-TF antibody (10 mg/kg of 43D8) relative to control.

### Example 11: Binding Affinity Assay

Kinetic measurements for the anti-TF antibodies were conducted on an Octet QK384 (Pall ForteBio, Fremont, CA, USA) or a Biacore (GE Healthcare Bio-Sciences).

ForteBio affinity measurements were performed generally as previously described (Estep et al., MAbs. 2013 Mar-Apr; 5(2):270-8). Briefly, ForteBio affinity measurements were performed by loading IgGs on-line onto AHC sensors. Sensors were equilibrated off-line in assay buffer for 30 min and then monitored on-line for 60 seconds for baseline establishment. Sensors with loaded IgGs were exposed to 100 nM antigen (human, cynomolgus, or mouse TF) for 3 min, afterwards they were transferred to assay buffer for 3 min for off-rate measurement. Alternatively, binding measurements were obtained by loading biotinylated TF monomer on SA sensors followed by exposure to 100 nM antibody Fab in solution. Kinetic data was analyzed and fitted using a 1:1 Langmuir binding model and the K_{D} was calculated by dividing the k_{off} by the kₒₙ. The K_{D} values of the TF antibodies measured by the Octet-based experiments are shown in Table 5.

For the Biacore-based measurements, the antibody was covalently coupled to a CM5 or C1 chip using an amine-coupling kit (GE Healthcare Bio-Sciences). Association between the anti-TF antibodies and a five-point three-fold titration of TF-His starting at 25 to 500 nM was measured for 300 sec. Subsequently, dissociation between the anti-TF antibody and TF-His was measured for up to 1800 sec. Kinetic data was analyzed and fitted globally using a 1:1 binding model. The K_{D} values of the TF antibodies measured by the Biacore-based experiments are shown in Table 5.

As shown in Table 5, the affinity of the antibodies for hTF, as indicated by K_{D}, is between 10⁻⁷ M and 10⁻¹¹ M. All anti-hTF antibodies are cross-reactive with cTF. In addition, all anti-hTF antibodies from groups 25 and 43 exhibit binding activity to mTF. The anti-hTF antibodies 25G, 25G1, 25G9, and 43D8 are cross-reactive with mTF. There are no other known human or humanized anti-hTF monoclonal antibodies that exhibit binding activity and cross-reactivity to mouse TF, indicating that the antibodies from groups 25 and 43 bind to a novel TF epitope.

TABLE 5
Antibody Kinetics

| | Human | Cynomolgus | Mouse | Human | Cynomolgus | Mouse |
|---|---|---|---|---|---|---|
| | K_{D} (nM) | K_{D} (nM) | K_{D} (nM) | K_{D} (nM) | K_{D} (nM) | K_{D} (nM) |
| Ab | [Biacore] | [Biacore] | [Biacore] | [ForteBio] | [ForteBio] | [ForteBio] |
| 1F | 0.31 | 0.26 | nd* | 1.28 | 1.43 | no binding* |
| 1G | nd* | nd* | nd* | 2.20 | 2.70 | nd* |
| 25A | 6.20 | 5.42 | nd* | 8.45 | 7.65 | 263 |
| 25A3 | 0.36 | 0.21 | nd* | 1.67 | 1.36 | 131 |
| 25A5 | 0.08 | 0.04 | nd* | 0.64 | 0.76 | 188 |
| 25G | 23.0 | 18.0 | nd* | 21.9 | 17.5 | 114 |
| 25G1 | 0.94 | 0.78 | 5.4 | 3.97 | 4.99 | 34.2 |
| 25G9 | 13.3 | 16.4 | 2.9 | 35.8 | 42.9 | 9.16 |
| 29D | nd* | nd* | nd* | 3.30 | 12.0 | nd |
| 29E | 0.47 | 5.06 | nd* | 2.32 | 15.0 | no binding* |
| 39A | 0.09 | 0.08 | nd* | 0.83 | 0.57 | no binding* |
| 43B | 1.75 | 5.64 | nd* | 2.40 | 3.40 | 161 |
| 43B1 | 0.07 | 0.12 | nd* | 0.96 | 1.05 | 72.1 |
| 43B7 | 0.14 | 0.24 | nd* | 0.86 | 0.94 | 360 |
| 43D | 2.09 | 5.66 | nd* | 3.84 | 4.12 | 281 |
| 43D7 | 0.06 | 0.12 | 21 | 1.02 | 1.11 | 41.4 |
| 43D8 | 0.15 | 0.39 | 2.4 | 1.61 | 1.96 | 6.12 |
| 43E | 1.46 | 5.69 | nd* | 2.52 | 4.07 | 121 |
| 43Ea | 1.60 | 6.42 | nd* | 2.28 | 2.71 | 140 |
| 54E | 0.42 | 1.83 | nd* | 1.59 | 4.16 | no binding* |

no binding*: no to weak binding, with no reportable K_{D}
nd*: not determined

### Example 12: Cell-Based Binding Assay

HCT116 cells with endogenous expression of human TF were obtained from the American Tissue Culture Collection (ATCC, Manassas, VA, USA) and were maintained as recommended. Flp-In-CHO cells expressing mouse TF were generated by transfection of Flp-In-CHO cells as recommended with a pcDNA5/FRT vector (ThermoFisher Scientific) encoding full-length mouse TF with a C-terminal FLAG tag. A mouse TF-positive CHO clone was isolated by limiting dilution in tissue culture-treated 96-well plates.

Cell-based antibody binding was assessed as previously described in Liao-Chan et al., PLoS One, 2015, 10:e0124708, which is incorporated by reference in its entirety. 1.2×10⁵ cells collected with Cellstripper (Mediatech, Manassas, VA, USA) were incubated with a twelve-point 1:3 dilution titration of anti-human TF IgG1 or Fab antibody starting at 250 nM or 100 nM for 2 hr on ice. After 2 washes, cells labeled with IgG1 or Fab were incubated for 30 min on ice with 150 nM of Goat Phycoerythrin (PE) F(ab′)₂ fragment goat anti-human IgG, Fcγ fragment specific (Jackson ImmunoResearch, West Grove, PA, USA) or FITC-labeled F(ab′)₂ fragment goat anti-human kappa (SouthernBiotech, Birmingham, AL, USA), respectively. After 2 washes, dead cells were labeled with TO-PRO-3 Iodide (ThermoFisher Scientific) and samples were analyzed on a CytoFLEX flow cytometer (Beckman Coulter, Brea, CA, USA) or Novocyte flow cytometer (ACEA Biosciences, San Diego, CA, USA). The median fluorescence intensities (MFIs) at each dilution were plotted and cell EC₅₀'s were derived using a 4-parameter binding model in Prism (GraphPad, La Jolla, CA, USA). The results of binding of anti-TF antibodies to human TF-positive HCT-116 cells are shown in international PCT application PCT/US2019/012427 and U.S. utility application Ser. No. 16/959,652, incorporated herein by reference in their entirety. The results of binding of anti-TF antibodies to CHO cells expressing mouse TF are shown in international PCT application PCT/US2019/012427 and U.S. utility application Ser. No. 16/959,652, incorporated herein by reference in their entirety.

All anti-hTF antibodies are shown in international PCT application PCT/US2019/012427 and U.S. utility application Ser. No. 16/959,652 exhibit high affinity to human TF-positive HCT-116 cells with an EC₅₀ ranging from about 687 pM to about 39 pM. Antibodies from groups 25 and 43 exhibit binding to CHO cells expressing mouse TF with an EC₅₀ ranging from about 455 nM to about 2.9 nM, are shown in international PCT application PCT/US2019/012427 and U.S. utility application Ser. No. 16/959,652, incorporated herein by reference in their entirety. The binding activity to mouse TF is a unique property of the anti-hTF antibodies (e.g., from groups 25 and 43). This is advantageous for pre-clinical studies of these antibodies with mouse models. In certain embodiments, binding affinity to mouse TF is an important property for selecting antibodies for inflammatory diseases, inflammation and fibrosis.

### Example 13: Thrombin Generation Assay (TGA)

The TGA assay was performed using the calibrated-automated-thrombogram (CAT) instrument manufactured and distributed by STAGO. The test method design was equivalent to a standard CAT assay measurement, except that the plasma source was NPP in citrate/CTI. The anti-TF antibodies were titrated at 0, 10, 50 and 100 nM and mixed with normal pooled plasma (NPP) collected in 11 mM citrate supplemented with 100 microgram/mL of corn trypsin inhibitor (citrate/CTI). Relipidated TF was added to a 96-well assay plate, followed by addition of the antibody/NPP mixture. After a 10-min incubation or directly after combining the relipidated TF with antibody/NPP, thrombin generation was initiated by the addition of calcium and the thrombin substrate. The STAGO software was used to report the following parameters: Peak IIa (highest thrombin concentration generated [nM]); Lag Time (time to IIa generation [min]); ETP (endogenous thrombin potential, area under the curve [nM×min]); and ttPeak (time to Peak IIa [min]). Percent peak thrombin generation (% Peak IIa) and percent endogenous thrombin potential (% ETP) in the presence of each antibody relative to a no antibody plasma control on the same plate were also reported.

The Peak IIa, Lag Time, ETP, ttPeak, % Peak IIa, and % ETP in the presence of each antibody selected from 1F, 25A, 25A3, 25G1, 29E, 39A, 43B1, 43D7, 43Ea, and 54E without antibody incubation prior to addition of calcium and thrombin substrate are shown in Table 6. The Peak IIa, Lag Time, ETP, ttPeak, % Peak IIa, and % ETP in the presence of each antibody selected from 1F, 25A, 25A3, 25G1, 29E, 39A, 43B1, 43D7, 43Ea, and 54E with 10 min antibody incubation prior to addition of calcium and thrombin substrate are shown in Table 7. The % Peak IIa in the presence of titrations of anti-TF antibodies without antibody incubation prior to addition of calcium and thrombin substrate are shown in international PCT application PCT/US2019/012427 and U.S. utility application Ser. No. 16/959,652, incorporated herein by reference in their entirety. The % Peak IIa in the presence of titrations of anti-TF antibodies with 10 min antibody incubation prior to addition of calcium and thrombin substrate are shown in international PCT application PCT/US2019/012427 and U.S. utility application Ser. No. 16/959,652, incorporated herein by reference in their entirety.

The % Peak IIa is greater than 90% in the presence of antibodies from group 25, including 25A, 25A3, and 25G1. The % ETP is greater than 100% in the presence of antibodies from group 25, including 25A, 25A3, and 25G1. The % Peak IIa is greater than 40% in the presence of antibodies from group 43, including 43B1, 43D7, and 43Ea. The % ETP is greater than 90% in the presence of antibodies from group 43, including 43B1, 43D7, and 43Ea.

This data indicates that antibodies from groups 25 and 43 allow normal thrombin generation, and therefore are not inhibitors of thrombin generation.

TABLE 6
Thrombin Generation Assay without Antibody Pre-Incubation

| | | Ab conc. | Peak IIa | Lag Time | ETP | ttPeak | % Peak | |
|---|---|---|---|---|---|---|---|---|
| Plate | Antibody | (nM) | (nM) | (mm) | (nM · min) | (mm) | IIa | % ETP |
| 1 | 1F | 100 | 29 | 25.9 | * | 37.9 | 7 | * |
| | | 50 | 32 | 27.2 | * | 36.8 | 8 | * |
| | | 10 | 83 | 12.1 | 1395 | 19.8 | 21 | 58 |
| 1 | 25A | 100 | 398 | 4.4 | 2610 | 7.1 | 99 | 108 |
| | | 50 | 399 | 4.2 | 2621 | 7.1 | 99 | 108 |
| | | 10 | 403 | 4.1 | 2555 | 6.8 | 100 | 106 |
| 1 | 25A3 | 100 | 405 | 3.9 | 2493 | 6.5 | 100 | 103 |
| | | 50 | 404 | 3.9 | 2495 | 6.6 | 100 | 103 |
| | | 10 | 401 | 4.2 | 2550 | 7.3 | 99 | 106 |
| 1 | 25G1 | 100 | 416 | 4.5 | 2626 | 7.1 | 103 | 109 |
| | | 50 | 416 | 4.5 | 2680 | 7.1 | 103 | 111 |
| | | 10 | 417 | 4.5 | 2635 | 7.0 | 103 | 109 |
| 1 | 29E | 100 | 99 | 17.3 | * | 26.4 | 25 | * |
| | | 50 | 107 | 14.4 | 1747 | 22.7 | 26 | 72 |
| | | 10 | 266 | 5.7 | 2189 | 10.0 | 66 | 91 |
| 1 | 39A | 100 | 26 | 28.9 | * | 40.1 | 6 | * |
| | | 50 | 30 | 30.5 | * | 40.0 | 7 | * |
| | | 10 | 82 | 12.1 | 1330 | 19.6 | 20 | 55 |
| 1 | Plasma | NA | 403 | 4.1 | 2417 | 6.8 | 100 | 100 |
| | ctrl. | | | | | | | |
| 2 | 43B1 | 100 | 221 | 5.2 | 2167 | 10.6 | 64 | 100 |
| | | 50 | 232 | 5.2 | 2195 | 10.3 | 67 | 101 |
| | | 10 | 299 | 4.9 | 2288 | 8.9 | 87 | 105 |
| 2 | 43D7 | 100 | 179 | 5.4 | 2094 | 11.8 | 52 | 96 |
| | | 50 | 202 | 5.3 | 2116 | 11.1 | 58 | 97 |
| | | 10 | 287 | 5.0 | 2263 | 9.0 | 83 | 104 |
| 2 | 43Ea | 100 | 300 | 4.6 | 2219 | 8.1 | 87 | 102 |
| | | 50 | 307 | 4.6 | 2234 | 8.1 | 89 | 103 |
| | | 10 | 328 | 5.0 | 2329 | 8.3 | 95 | 107 |
| 2 | 54E | 100 | 68 | 14.8 | 1175 | 23.9 | 20 | 54 |
| | | 50 | 154 | 8.9 | 2019 | 15.9 | 44 | 93 |
| | | 10 | 307 | 5.7 | 2307 | 9.6 | 89 | 106 |
| 2 | Isotype | 100 | 348 | 5.0 | 2415 | 8.3 | 101 | 111 |
| | | 50 | 347 | 5.0 | 2360 | 8.0 | 101 | 109 |
| | | 10 | 346 | 4.3 | 2260 | 7.6 | 100 | 104 |
| 2 | Plasma | NA | 345 | 4.7 | 2171 | 7.8 | 100 | 100 |
| | ctrl. | | | | | | | |

* Groups with “No Tail Found” Errors when the software cannot calculate the ETP.

TABLE 7
Thrombin Generation Assay with 10 min Antibody Pre-Incubation

| | | Ab conc. | Peak IIa | Lag Time | ETP | ttPeak | % Peak | |
|---|---|---|---|---|---|---|---|---|
| Plate | Antibody | (nM) | (nM) | (mm) | (nM · min) | (mm) | IIa | % ETP |
| 1 | 1F | 100 | 17 | 30.3 | * | 42.0 | 7 | * |
| | | 50 | 20 | 27.6 | * | 38.9 | 7 | * |
| | | 10 | 27 | 18.8 | 540 | 28.6 | 10 | 31 |
| 1 | 25A | 100 | 285 | 3.3 | 1898 | 6.7 | 108 | 110 |
| | | 50 | 284 | 3.3 | 1887 | 6.6 | 107 | 110 |
| | | 10 | 277 | 3.3 | 1842 | 6.7 | 105 | 107 |
| 1 | 25A3 | 100 | 277 | 3.1 | 1785 | 6.3 | 105 | 104 |
| | | 50 | 275 | 3.2 | 1824 | 6.4 | 104 | 106 |
| | | 10 | 278 | 3.2 | 1827 | 6.6 | 105 | 106 |
| 1 | 25G1 | 100 | 293 | 3.3 | 1827 | 6.4 | 111 | 106 |
| | | 50 | 301 | 3.3 | 1853 | 6.3 | 114 | 108 |
| | | 10 | 302 | 3.3 | 1891 | 6.3 | 114 | 110 |
| 1 | 29E | 100 | 68 | 15.1 | 1098 | 25.3 | 26 | 64 |
| | | 50 | 70 | 14.2 | 1168 | 24.3 | 27 | 68 |
| | | 10 | 78 | 10.4 | 1254 | 20.2 | 30 | 73 |
| 1 | 39A | 100 | 17 | 28.0 | * | 40.2 | 7 | * |
| | | 50 | 17 | 28.4 | 346 | 38.9 | 7 | 20 |
| | | 10 | 25 | 20.8 | 482 | 30.7 | 9 | 28 |
| 1 | Plasma | NA | 264 | 3.3 | 1720 | 6.8 | 100 | 100 |
| | ctrl. | | | | | | | |
| 2 | 43B1 | 100 | 152 | 3.2 | 1712 | 9.3 | 58 | 98 |
| | | 50 | 163 | 3.2 | 1797 | 9.0 | 62 | 103 |
| | | 10 | 200 | 3.2 | 1788 | 8.1 | 76 | 103 |
| 2 | 43D7 | 100 | 124 | 3.6 | 1656 | 10.3 | 47 | 95 |
| | | 50 | 128 | 3.6 | 1677 | 10.3 | 49 | 96 |
| | | 10 | 178 | 3.6 | 1745 | 8.8 | 68 | 100 |
| 2 | 43Ea | 100 | 239 | 2.9 | 1820 | 6.9 | 91 | 104 |
| | | 50 | 227 | 2.9 | 1791 | 7.1 | 87 | 103 |
| | | 10 | 247 | 3.2 | 1825 | 7.0 | 94 | 105 |
| 2 | 54E | 100 | 29 | 22.1 | 580 | 32.3 | 11 | 33 |
| | | 50 | 35 | 18.3 | 680 | 28.4 | 13 | 39 |
| | | 10 | 112 | 6.1 | 1530 | 13.4 | 43 | 88 |
| 2 | Isotype | 100 | 288 | 3.2 | 1888 | 6.6 | 110 | 108 |
| | | 50 | 285 | 3.2 | 1879 | 6.6 | 109 | 108 |
| | | 10 | 273 | 3.2 | 1804 | 6.6 | 104 | 104 |
| 2 | Plasma | NA | 262 | 3.2 | 1742 | 6.9 | 100 | 100 |
| | ctrl. | | | | | | | |

* Groups with “No Tail Found” Errors when the software cannot calculate the ETP.

### Example 14: FXa Conversion Assay

To evaluate the ability of TF:FVIIa to convert FX into FXa in the presence of human antibodies against TF, 5×10⁴ MDA-MB-231 cells (ATCC, Manassas, VA, USA) were plated into tissue culture-treated black 96-well plates (Greiner Bio-One, Monroe, NC, USA). After removal of the cell culture media and addition of a final concentration of 200 nM of FX in a HEPES buffer with 1.5 mM CaCl₂), cells were incubated with a titration of the antibodies for 15 min at 37° C. Upon reconstitution of the binary TF:FVIIa complex with a final concentration of 20 nM of FVIIa, cells were incubated for 5 min at 37° C. After quenching the reaction with ethylenediaminetetraacetic acid (EDTA), generated FXa was measured with 50 pM of SN-7 6-amino-1-naphthalenesulfonamide-based fluorogenic substrate (Haematologic Technologies, Essex Junction, VT, USA) on an Envision plate reader equipped with an Umbelliferone 355 excitation filter, an Umbelliferone 460 emission filter, and a LANCE/DELFIA top mirror (Perkin Elmer, Waltham, MA, USA). FXa conversion percentages (% FXa) in the presence of an anti-TF antibody titration relative to a no-antibody control are summarized in Table 8 and plotted in international PCT application PCT/US2019/012427 and U.S. utility application Ser. No. 16/959,652, incorporated herein by reference in their entirety.

The FXa conversion percentage ranges from about 7800 to about 12000 in presence of different concentrations of antibodies from groups 25 and 43, including 25A, 25A3, 25G, 25G1, 25G5, 25G9, 43B, 43B1, 43B7, 43D, 43D37, 431D8, 43E, and 43Ea.

This data indicates that anti-TF antibodies from groups 25 and 43 do not inhibit TF:FVIIa mediated FXa conversion from FX. This data also indicates that anti-TF antibodies from groups 25 and 43 have a human TF binding site that is distinct from the human TF binding site bound by FX.

TABLE 8
% FXa conversion

% FXa

| Antibody | 12.5 nM | 25 nM | 50 nM | 100 nM |
|---|---|---|---|---|
| 1F | 49 | 40 | 37 | 38 |
| 1G | 55 | 48 | 41 | 41 |
| 25A | 87 | 81 | 94 | 89 |
| 25A3 | 89 | 89 | 93 | 96 |
| 25A5 | 82 | 85 | 78 | 89 |
| 25G | 99 | 109 | 102 | 116 |
| 25G1 | 101 | 96 | 99 | 108 |
| 25G9 | 98 | 97 | 104 | 117 |
| 29D | 85 | 77 | 75 | 75 |
| 29E | 81 | 68 | 63 | 66 |
| 39A | 39 | 38 | 37 | 39 |
| 43B | 113 | 109 | 105 | 105 |
| 43B1 | 106 | 108 | 108 | 112 |
| 43B7 | 113 | 104 | 108 | 112 |
| 43D | 115 | 109 | 104 | 106 |
| 43D7 | 110 | 103 | 102 | 103 |
| 43D8 | 120 | 112 | 107 | 111 |
| 43E | 85 | 89 | 97 | 98 |
| 43Ea | 108 | 103 | 106 | 101 |
| 54E | 53 | 44 | 41 | 42 |
| 5G9 | 37 | 33 | 30 | 30 |
| Isotype ctrl | 93 | 95 | 89 | 97 |

### Example 15: FVIIa Competition Assay

FVII-Fc conjugates were generated using Alexa Fluor 488 5-sulfo-dichlorophenol esters (ThermoFisher Scientific). Excess Alexa Fluor dye was removed from the conjugate preparations by gel filtration (ThermoFisher Scientific).

To evaluate competition between FVIJa and the human antibodies against TF, TF-positive MIDA-MB-231 cells (ATCC, Manassas, VA, USA) were first incubated for 1 hr on ice with a titration of the human antibodies against TF. Subsequently, a final concentration of 20 nM of FVII-Fc conjugated to Alexa488 was added to the antibody cell mixture. After another 1 hr incubation on ice, cells were washed, stained with a viability dye, and analyzed by flow cytometry. The Alexa488 fluorescence data from viable cells was summarized using median fluorescence intensity. FVII-Fc binding was summarized with % FVII-Fc binding=[MFI_{antibody labeled cells}—MFI_{unstained} cells]/[MFI_{IgG1 control labeled cells}—MFI_{unstained} cells]. Percentage of FVIIa binding (% FVIIa) in the presence of an anti-TF antibody titration relative to a no-antibody control is summarized in Table 9 and are shown in international PCT application PCT/US2019/012427 and U.S. utility application Ser. No. 16/959,652, incorporated herein by reference in their entirety.

The FVIIa binding percentage ranges from about 76% to about 102% in the presence of antibodies of different concentrations from groups 25 and 43, including 25A, 25A3, 25G, 25G1, 25G5, 25G9, 43B, 43B1, 43B7, 43D, 43D7, 43D8, 43E, and 43Ea.

This data indicates that anti-TF antibodies from groups 25 and 43 do not compete for binding to human TF with FVIIa. This data also indicates that anti-TF antibodies from groups 25 and 43 have a human TF binding site that is distinct from the human TF binding site bound by FVIIa.

TABLE 9
Competition of Anti-TF Antibody with FVIIa

% FVIIa

| Antibody | 9.25 nM | 28 nM | 83 nM | 250 nM |
|---|---|---|---|---|
| 1F | 7 | 7 | 7 | 6 |
| 1G | 7 | 7 | 7 | 6 |
| 25A | 100 | 101 | 97 | 98 |
| 25A3 | 90 | 87 | 88 | 87 |
| 25A5 | 76 | 79 | 77 | 80 |
| 25G | 97 | 96 | 93 | 92 |
| 25G1 | 97 | 93 | 94 | 95 |
| 25G9 | 93 | 93 | 91 | 89 |
| 29D | 6 | 4 | 3 | 3 |
| 29E | 5 | 3 | 2 | 2 |
| 39A | 2 | 2 | 2 | 2 |
| 43B | 99 | 95 | 93 | 91 |
| 43B1 | 97 | 95 | 93 | 91 |
| 43B7 | 98 | 98 | 97 | 97 |
| 43D | 102 | 100 | 98 | 94 |
| 43D7 | 101 | 102 | 100 | 101 |
| 43D8 | 100 | 99 | 98 | 96 |
| 43E | 95 | 92 | 91 | 89 |
| 43Ea | 93 | 91 | 92 | 89 |
| 54E | 11 | 3 | 3 | 2 |
| Isotype | 99 | 98 | 97 | 99 |

### Example 16: TF Signaling Assay

IL-8 and GM-CSF protein levels were measured as described previously in Hjortoe et al., Blood, 2004, 103:3029-3037. TF-positive MDA-MB-231 cells (ATCC, Manassas, VA, USA) that underwent a 2 hr serum starvation with Leibovitz's L-15 medium were incubated with an 8-point 1:2.5 titration starting at 100 nM of anti-TF antibody. After 30 min at 37° C., FVIIa (NovoSeven RT, Novo Nordisk, Bagsvaerd, Denmark) was added to the cells at a final concentration of 20 nM. 5 hr later cell culture supernatants were harvested and analyzed by ELISA for IL8 or GM-CSF as recommended (R&D Biosystems, Minneapolis, MN, USA). A standard curve using recombinant IL8 or GM-CSF (R&D Biosystems, Minneapolis, MN, USA) was used in Prism to calculate cytokine concentration in the cell culture supernatants. Percent IL8 and GM-CSF (% IL8 and % GM-CSF) at reported antibody concentration were calculated relative to a no antibody control. The concentration of IL8 with the anti-TF antibody titration are shown in international PCT application PCT/US2019/012427 and U.S. utility application Ser. No. 16/959,652, incorporated herein by reference in their entirety and the % IL8 at different antibodies concentrations are shown in Table 10. The concentration of GM-CSF with the anti-TF antibody titration is shown in international PCT application PCT/US2019/012427 and U.S. utility application Ser. No. 16/959,652, incorporated herein by reference in their entirety and the % IL8 at different antibodies concentrations are shown in Table 11.

IL8 concentrations were reduced by more than 75% in the presence of the anti-TF antibodies at concentrations greater than or equal to 6.4 nM. GM-CSF concentrations were reduced by more than 60% in the presence of the anti-TF antibodies at concentrations greater than or equal to 6.4 nM.

This data indicates that all tested anti-TF antibodies inhibit FVIIa-dependent TF signaling.

TABLE 10
Inhibition of IL8

| | % IL8 | | | | |
|---|---|---|---|---|---|
| Antibody | 100 nM | 40 nM | 16 nM | 6.4 nM | 2.56 nM |
| 1F | 2 | 2 | 2 | 3 | 18 |
| 1G | 2 | 2 | 3 | 4 | 26 |
| 25A | 9 | 8 | 10 | 11 | 43 |
| 25A3 | 8 | 8 | 8 | 9 | 47 |
| 25A5 | 6 | 7 | 7 | 14 | 70 |
| 25G | 9 | 10 | 16 | 22 | 60 |
| 25G1 | 9 | 8 | 9 | 12 | 46 |
| 25G9 | 13 | 14 | 15 | 22 | 51 |
| 29D | 1 | 2 | 2 | 6 | 27 |
| 29E | 2 | 2 | 2 | 5 | 33 |
| 39A | 3 | 2 | 2 | 6 | 52 |
| 43B | 4 | 4 | 5 | 11 | 50 |
| 43B1 | 5 | 5 | 6 | 12 | 56 |
| 43B7 | 4 | 4 | 8 | 15 | 55 |
| 43D | 5 | 5 | 7 | 21 | 58 |
| 43D7 | 5 | 4 | 5 | 11 | 48 |
| 43D8 | 5 | 5 | 5 | 21 | 67 |
| 43E | 5 | 5 | 6 | 15 | 49 |
| 43Ea | 6 | 6 | 6 | 14 | 52 |
| 54E | 2 | 2 | 3 | 8 | 48 |
| Control | 106 | 108 | 84 | 88 | 90 |

TABLE 11
Inhibition of GM-CSF

| | % GM-CSF | | | | |
|---|---|---|---|---|---|
| Antibody | 100 nM | 40 nM | 16 nM | 6.4 nM | 2.56 nM |
| 1F | 6 | 6 | 6 | 8 | 27 |
| 1G | 7 | 7 | 7 | 9 | 34 |
| 25A | 22 | 19 | 22 | 24 | 57 |
| 25A3 | 20 | 19 | 19 | 20 | 59 |
| 25A5 | 12 | 15 | 14 | 18 | 72 |
| 25G | 19 | 18 | 32 | 39 | 77 |
| 25G1 | 17 | 16 | 17 | 18 | 48 |
| 25G9 | 25 | 26 | 26 | 34 | 60 |
| 29D | 5 | 6 | 7 | 15 | 38 |
| 29E | 6 | 6 | 5 | 9 | 33 |
| 39A | 7 | 5 | 5 | 8 | 42 |
| 43B | 14 | 13 | 12 | 21 | 59 |
| 43B1 | 11 | 11 | 13 | 16 | 50 |
| 43B7 | 11 | 11 | 13 | 17 | 50 |
| 43D | 12 | 11 | 13 | 24 | 56 |
| 43D7 | 10 | 10 | 9 | 15 | 45 |
| 43D8 | 12 | 11 | 11 | 24 | 57 |
| 43E | 14 | 15 | 15 | 21 | 61 |
| 43Ea | 14 | 15 | 14 | 21 | 65 |
| 54E | 5 | 5 | 5 | 10 | 38 |
| Control | 105 | 111 | 94 | 86 | 88 |

### Example 17: Antibody Competition Assay

Alexa Fluor antibodies were generated using Alexa Fluor 488 5-sulfo-dichlorophenol esters (ThermoFisher Scientific). Excess Alexa Fluor dye was removed from the antibody dye conjugate preparations by gel filtration (ThermoFisher Scientific).

To evaluate competition between a first human antibody against TF and 25A, TF-positive A431 cells (ATCC, Manassas, VA, USA) were first incubated for 1 hr on ice with a titration of the first human antibody against TF. Subsequently, a final concentration of 20 nM of 25A conjugated to Alexa488 was added to the antibody cell mixture. After another 1 hr incubation on ice, cells were washed, stained with a viability dye, and analyzed by flow cytometry. The Alexa488 fluorescence data from viable cells was summarized using median fluorescence intensity. 25A binding was summarized with % 25A binding=[MFI_{antibody labeled cells}—MFI_{unstained} cells]/[MFI_{IgG1 control labeled cells}−MFI_{unstained} cells].

To evaluate competition between a first human antibody against TF and 43Ea, TF-positive A431 cells (ATCC, Manassas, VA, USA) were first incubated for 1 hr on ice with a titration of the first human antibody against TF. Subsequently, a final concentration of 20 nM of 43Ea conjugated to Alexa488 was added to the antibody cell mixture. After another 1 hr incubation on ice, cells were washed, stained with a viability dye, and analyzed by flow cytometry. The Alexa488 fluorescence data from viable cells was summarized using median fluorescence intensity. 43Ea binding was summarized with % 43Ea binding=[MFI_{antibody labeled cells}−MFI_{unstained} cells]/[MFI_{IgG1 control labeled cells}−MFI_{unstained} cells].

% 25A binding and % 43Ea binding are shown in Table 12. Antibodies from group 25 and group 43 reduced the % 25A binding and % 43Ea binding to less than 10%.

This data indicates that antibodies of group 25 and antibodies of group 43 compete with each other for binding to human TF, and may bind the same or an overlapping epitope of human TF.

TABLE 12
Competition of Anti-TF Antibody
with Antibody Clone 25A or 43Ea

| Antibody (100 nM) | % 25A binding | % 43Ea binding |
|---|---|---|
| 1F | 95 | 77 |
| 1G | 75 | 58 |
| 25A | 3 | 1 |
| 25G | 7 | 3 |
| 29D | 70 | 64 |
| 29E | 96 | 85 |
| 39A | 99 | 96 |
| 43B | 0 | 0 |
| 43D | 0 | 0 |
| 43E | 0 | 0 |
| 54E | 99 | 96 |
| Isotype | 100 | 100 |

### Example 18: Cell Viability Assay

To evaluate internalization of the anti-TF antibodies, a cytotoxicity assay was conducted. Briefly, cells were plated in 384-well plates (Greiner Bio-One, Monroe, NC, USA) at 4×10³ cells per well in 40 μl of media. Antibodies and secondary anti-human Fe antibodies conjugated to the tubulin inhibitor mono-methyl auristatin F (MMAF) (Moradec, San Diego, CA, USA) were serially diluted starting at 5 and 30 nM, respectively. Plates were incubated for 3 days, followed by lysis in CellTiter-Glo (CTG) assay reagent (Promega, Madison, WI, USA). CTG luminescence was measured on an Envision plate reader and the mean and standard deviation of 4 replicates graphed in Prism. For each anti-TF antibody, the IC₅₀ and its associated 95% confidence interval were calculated in Prism using a 4-parameter binding model.

The cell viability as indicated by the level of luminescence and the calculated IC₅₀ is shown in international PCT application PCT/US2019/012427 and U.S. utility application Ser. No. 16/959,652, incorporated herein by reference in their entirety.

This data indicates that all anti-TF antibodies tested from groups 1, 25, 29, 39, 43, and 54 were effective in reducing the viability of TF-positive A431 cells.

### Example 19: Thrombin Generation Assay (TGA)

The TGA assay was performed using the calibrated-automated-thrombogram (CAT) instrument manufactured and distributed by STAGO. The test method design was equivalent to a standard CAT assay measurement, except that the plasma source was normal pooled plasma (NPP) in citrate supplemented with corn trypsin inhibitor (citrate/CTI). The anti-TF antibodies were titrated at 0, 10, 50 and 100 nM and mixed with normal pooled plasma (NPP) collected in 11 mM citrate supplemented with 100 microgram/mL of corn trypsin inhibitor (citrate/CTI). Relipidated TF was added to a 96-well assay plate, followed by addition of the antibody/NPP mixture. After a 10-min incubation or directly after combining the relipidated TF with antibody/NPP, thrombin generation was initiated by the addition of calcium and the thrombin substrate. The STAGO software was used to report the following parameters: Peak IIa (highest thrombin concentration generated [nM]); Lag Time (time to IIa generation [min]); ETP (endogenous thrombin potential, area under the curve [nM×min]); and ttPeak (time to Peak IIa [min]). Percent peak thrombin generation (% Peak IIa) and percent endogenous thrombin potential (% ETP) in the presence of each antibody relative to a no antibody plasma control on the same plate were also reported.

The Peak IIa, Lag Time, ETP, ttPeak, % Peak IIa, and % ETP in the presence of each antibody selected from 25A, 25A3, 25A5, 39A, 43B1, 43D7, 43Ea, and M1593 without antibody incubation prior to addition of calcium and thrombin substrate are shown in Table 37. The Peak IIa, Lag Time, ETP, ttPeak, % Peak IIa, and % ETP in the presence of each antibody selected from 25A, 25A3, 25A5, 39A, 43B1, 43D7, 43Ea, and M1593 with 10 min antibody incubation prior to addition of calcium and thrombin substrate are shown in Table 38. The % Peak IIa in the presence of titrations of anti-TF antibodies without antibody incubation prior to addition of calcium and thrombin substrate are shown in international PCT application PCT/US2019/012427 and U.S. utility application Ser. No. 16/959,652, incorporated herein by reference in their entirety. The % Peak IIa in the presence of titrations of anti-TF antibodies with 10 min antibody incubation prior to addition of calcium and thrombin substrate is shown in international PCT application PCT/US2019/012427 and U.S. utility application Ser. No. 16/959,652, incorporated herein by reference in their entirety. The M1593 antibody has a VH sequence of SEQ ID NO:821 and VL sequence of SEQ ID NO:822.

The % Peak IIa is 95% or greater in the presence of antibodies from group 25, including 25A, 25A3, and 25A5 without antibody pre-incubation. The % Peak IIa is 100% or greater in the presence of antibodies from group 25, including 25A, 25A3, and 25A5 with 10 min antibody pre-incubation. The % ETP is 99% or greater in the presence of the tested antibodies from group 25.

The % Peak IIa is greater than 50% but equal to or less than 96% in the presence of antibodies from group 43, including 43B1, 43D7, and 43Ea and anti-TF antibody M1593 without antibody pre-incubation. The % Peak IIa is greater than 40% but equal to or less than 93% in the presence of antibodies from group 43, including 43B1, 43D7, and 43Ea and anti-TF antibody M1593 with 10 min antibody pre-incubation. The % ETP is 92% or greater in the presence of the tested antibodies from group 43 and M1593 antibody.

This data indicates that antibodies from groups 25 and 43 allow normal thrombin generation, and therefore are not inhibitors of thrombin generation. The percent peak thrombin generation (% Peak IIa) is greater in the presence of antibodies of group 25 compared to antibodies of group 43 and M1593 antibody.

TABLE 37
Thrombin Generation Assay without Antibody Pre-Incubation

| | | Ab conc. | Peak IIa | Lag Time | ETP | ttPeak | % Peak | |
|---|---|---|---|---|---|---|---|---|
| Plate | Antibody | (nM) | (nM) | (mm) | (nM · min) | (mm) | IIa | % ETP |
| 3 | 25A | 100 | 334 | 5.0 | 2390 | 8.7 | 96 | 105 |
| | | 50 | 335 | 5.0 | 2380 | 8.7 | 96 | 104 |
| | | 10 | 333 | 5.0 | 2387 | 8.6 | 95 | 104 |
| 3 | 25A3 | 100 | 343 | 5.0 | 2405 | 8.4 | 98 | 105 |
| | | 50 | 349 | 5.0 | 2433 | 8.4 | 100 | 106 |
| | | 10 | 350 | 5.0 | 2426 | 8.0 | 100 | 106 |
| 3 | 25A5 | 100 | 342 | 5.1 | 2393 | 8.5 | 98 | 105 |
| | | 50 | 344 | 4.8 | 2317 | 8.1 | 98 | 101 |
| | | 10 | 343 | 4.7 | 2270 | 8.0 | 98 | 99 |
| 3 | 39A | 100 | 22 | 38.1 | * | 48.3 | 6 | * |
| | | 50 | 29 | 33.1 | * | 43.2 | 8 | * |
| | | 10 | 84 | 12.4 | 1332 | 20.7 | 24 | 58 |
| 3 | 43B1 | 100 | 223 | 4.8 | 2111 | 10.0 | 64 | 92 |
| | | 50 | 239 | 4.9 | 2134 | 9.9 | 68 | 93 |
| | | 10 | 303 | 5.1 | 2318 | 9.1 | 87 | 101 |
| 3 | 43D7 | 100 | 186 | 5.6 | 2105 | 12.2 | 53 | 92 |
| | | 50 | 216 | 5.5 | 2183 | 11.3 | 62 | 96 |
| | | 10 | 301 | 5.4 | 2338 | 9.3 | 86 | 102 |
| 3 | 43Ea | 100 | 302 | 5.1 | 2347 | 9.1 | 87 | 103 |
| | | 50 | 308 | 5.1 | 2392 | 8.8 | 88 | 105 |
| | | 10 | 336 | 4.5 | 2305 | 7.8 | 96 | 101 |
| 3 | M1593 | 100 | 242 | 5.1 | 2235 | 10.4 | 69 | 98 |
| | | 50 | 270 | 5.1 | 2282 | 9.8 | 77 | 100 |
| | | 10 | 322 | 5.1 | 2368 | 8.8 | 92 | 104 |
| 3 | Isotype | 100 | 347 | 5.0 | 2319 | 8.1 | 99 | 101 |
| | | 50 | 348 | 5.0 | 2324 | 8.1 | 100 | 102 |
| | | 10 | 348 | 5.0 | 2326 | 8.3 | 100 | 102 |
| 3 | Plasma | NA | 349 | 4.7 | 2285 | 7.7 | 100 | 100 |
| | ctrl. | | | | | | | |

* Groups with “No Tail Found” Errors when the software cannot calculate the ETP.

TABLE 38
Thrombin Generation Assay with 10 min Antibody Pre-Incubation

| | | Ab conc. | Peak IIa | Lag Time | ETP | ttPeak | % Peak | |
|---|---|---|---|---|---|---|---|---|
| Plate | Antibody | (nM) | (nM) | (mm) | (nM · min) | (mm) | IIa | % ETP |
| 3 | 25A | 100 | 274 | 3.3 | 1879 | 7.0 | 103 | 106 |
| | | 50 | 279 | 3.3 | 1876 | 7.0 | 105 | 106 |
| | | 10 | 280 | 3.6 | 1872 | 7.0 | 105 | 106 |
| 3 | 25A3 | 100 | 290 | 3.4 | 1906 | 6.8 | 109 | 108 |
| | | 50 | 291 | 3.6 | 1925 | 6.8 | 109 | 109 |
| | | 10 | 287 | 3.3 | 1886 | 6.8 | 108 | 107 |
| 3 | 25A5 | 100 | 286 | 3.7 | 1883 | 7.0 | 107 | 107 |
| | | 50 | 277 | 3.7 | 1803 | 7.0 | 104 | 102 |
| | | 10 | 278 | 3.7 | 1808 | 7.0 | 104 | 102 |
| 3 | 39A | 100 | 17 | 32.1 | * | 43.2 | 6 | * |
| | | 50 | 21 | 29.0 | * | 39.7 | 8 | * |
| | | 10 | 30 | 20.9 | * | 30.8 | 11 | * |
| 3 | 43B1 | 100 | 156 | 3.6 | 1701 | 9.3 | 58 | 96 |
| | | 50 | 148 | 3.3 | 1667 | 9.6 | 55 | 94 |
| | | 10 | 203 | 3.7 | 1776 | 8.2 | 76 | 101 |
| 3 | 43D7 | 100 | 120 | 3.7 | 1633 | 10.8 | 45 | 92 |
| | | 50 | 131 | 3.7 | 1724 | 10.4 | 49 | 98 |
| | | 10 | 197 | 3.7 | 1784 | 8.8 | 74 | 101 |
| 3 | 43Ea | 100 | 244 | 3.3 | 1817 | 7.3 | 91 | 103 |
| | | 50 | 246 | 3.3 | 1833 | 7.3 | 92 | 104 |
| | | 10 | 247 | 3.3 | 1779 | 7.1 | 93 | 101 |
| 3 | M1593 | 100 | 160 | 3.7 | 1737 | 9.4 | 60 | 98 |
| | | 50 | 165 | 3.7 | 1739 | 9.3 | 62 | 99 |
| | | 10 | 224 | 3.7 | 1807 | 8.0 | 84 | 102 |
| 3 | Isotype | 100 | 279 | 3.7 | 1829 | 7.2 | 105 | 104 |
| | | 50 | 283 | 3.7 | 1839 | 7.0 | 106 | 104 |
| | | 10 | 279 | 3.7 | 1814 | 7.1 | 105 | 103 |
| 3 | Plasma | NA | 267 | 3.7 | 1766 | 7.2 | 100 | 100 |
| | ctrl. | | | | | | | |

* Groups with “No Tail Found” Errors when the software cannot calculate the ETP.

### Example 20: Synthesis of Antibody-Drug Conjugates (ADCs)

Antibody-Drug Conjugates (ADCs) were synthesized as described in Behrens et al., Mol Pharm, 2015, 12:3986-98. 5 mg/mL of antibody in phosphate-buffered saline (PBS), pH 7.4 was reduced with 2.5 molar equivalents of Tris(2-carboxyehtyl)phosphine. After 2 hr at 37° C., the partially reduced antibody was cooled to room temperature and conjugated for 1 hr to 3 to 5 molar equivalents of MC-vc-PAB-MMAE (maleimidocaproyl-valine-citrulline-p-aminobenzoyloxycarbonyl-monomethyl auristatin E). The reaction was buffer exchanged into PBS to remove small molecular weight reagents. The drug-antibody ratio (DAR) of the resulting ADCs was 3-4. The DAR was determined with the following formula: Absorbance (248 nm)/Absorbance (280 nm)=(n×Ex_{PAB[248 nm]}+Ex_{antibody[248 nm]})/(n×Ex_{PAB[280 nm]} +Ex_{antibody[280 nm]}) with n as a variable for the DAR and Ex as the extinction coefficients of PAB and the antibody. Hydrophobic interaction chromatography and size exclusion chromatography were used to corroborate the absorbance-based DAR estimation and to ensure the ADC preparation was at least 95% monomeric, respectively.

### Example 21: Cytotoxicity Assays of Antibody-Drug Conjugates (ADCs)

To evaluate cytotoxicity of ADCs, TF-positive A431 and HPAF-II cells were plated in 384-well plates (Greiner Bio-One, Monroe, NC, USA) at 4×10³ cells per well in 40 μL of media. Anti-TF antibodies conjugated to MC-vc-PAB-MMAE were serially diluted starting at 5 nM. Plates were incubated for 3 to 4 days, followed by lysis in CellTiter-Glo (CTG) assay reagent (Promega, Madison, WI, USA). CTG luminescence was measured on an Envision plate reader and the mean and standard deviation of 4 replicates were graphed in Prism. For each ADC, the IC₅₀ and its associated 95% confidence interval were calculated in Prism using a 4-parameter binding model.

The cell viability as indicated by CTG luminescence and the calculated IC₅₀ in TF-positive A431 and HPAF-II cells is shown in international PCT application PCT/US2019/012427 and U.S. utility application Ser. No. 16/959,652, incorporated herein by reference in their entirety. ADCs comprising anti-TF antibodies from groups 25, 43, and 39 conjugated to MC-vc-PAB-MMAE resulted in cytotoxicity in TF-positive A431 and HPAF-II cells.

This data indicates that anti-TF antibody-drug conjugates reduced the viability of TF-positive cells in vitro.

### Example 22: Binding Affinity Assay for Pig TF

The ability of certain antibodies was tested for binding to pig TF. For pig TF Biacore-based measurements, a given anti-TF antibody was captured by an anti-human IgG antibody covalently coupled to a CM5 chip (GE Healthcare Bio-Sciences). Association between the anti-TF antibodies and a five-point three-fold titration of pig TF-His starting at 100 nM was measured for 180 to 240 sec. Subsequently, dissociation between the anti-TF antibody and TF-His was measured for 1800 sec. Kinetic data was analyzed and fitted globally using a 1:1 binding model. The K_{D} values of the indicated TF antibodies measured by the Biacore-based experiments are shown in Table 40.

As shown in Table 40, anti-hTF antibodies from groups 25 and 43, 25G9 and 43D8, exhibit binding activity and cross-reactivity to pig TF.

TABLE 40
Antibody kinetics for pig TF

| Ab | Pig K_{D} (nM) [standard deviation] |
|---|---|
| 1G | no binding* |
| 29D | no binding* |
| 25G9 | 3.31 [0.08] |
| 43D8 | 12.9 [0.03] |

no binding*: no binding to weak binding, with no reportable K_{D}

### Example 23: Cell-Based Binding Assay

Human TF-positive cancer cell lines A431 and MDA-MB-231 and Macaca mulatta TF-positive cell line RF/6A were obtained from the American Tissue Culture Collection (ATCC, Manassas, VA, USA) and were maintained as recommended.

Cell-based antibody binding was assessed as previously described in Liao-Chan et al., PLoS One, 2015, 10:e0124708, which is incorporated by reference in its entirety. 1.2×10⁵ cells collected with Cellstripper (Mediatech, Manassas, VA, USA) were incubated with a twelve-point 1:3 dilution titration of anti-human TF IgG1 antibody starting at 250 nM or 100 nM for 2 hr on ice. After 2 washes, cells labeled with IgG1 antibody were incubated for 30 min on ice with 150 nM of Goat Phycoerythrin (PE) F(ab′)₂ fragment goat anti-human IgG, Fcγ fragment specific (Jackson ImmunoResearch, West Grove, PA, USA) or FITC-labeled F(ab′)₂ fragment goat anti-human kappa (SouthemfBiotech, Birmingham, AL, USA), respectively. After 2 washes, dead cells were labeled with TO-PRO-3 Iodide (ThermoFisher Scientific) and samples were analyzed on a CytoFLEX flow cytometer (Beckman Coulter, Brea, CA, USA) or Novocyte flow cytometer (ACEA Biosciences, San Diego, CA, USA). The median fluorescence intensities (MFIs) at each dilution were plotted and cell EC₅₀'s were derived using a 4-parameter binding model in Prism (GraphPad, La Jolla, CA, USA). Antibodies that does not substantially affect FX conversion (i.e. 25A, 25A3, 25G1, 43B1, 43D7 and 43Ea) and antibodies that inhibited FX conversion by more than 50% (i.e. 1F, 29E, 39A and 54E) were included in the assay. The results of binding of anti-TF antibodies to human TF-positive A431 cells are shown in international PCT application PCT/US2019/012427 and U.S. utility application Ser. No. 16/959,652, incorporated herein by reference in their entirety. The results of binding of anti-TF antibodies to human TF-positive MDA-MB-231 cells are shown in international PCT application PCT/US2019/012427 and U.S. utility application Ser. No. 16/959,652, incorporated herein by reference in their entirety.

All tested anti-hTF antibodies in FIG. 12A of PCT application PCT/US2019/012427 and U.S. utility application Ser. No. 16/959,652 exhibit high affinity to human TF-positive A431 cells with an EC₅₀ ranging from about 1.50 nM to about 0.34 nM. An IgG1 isotype control did not bind A431 cells (no binding, nb). All tested anti-hTF antibodies in FIG. 12B of international PCT application PCT/US2019/012427 and U.S. utility application Ser. No. 16/959,652 exhibit high affinity to human TF-positive MDA-MB-231 cells with an EC₅₀ ranging from about 1.50 nM to about 0.06 nM. An IgG1 isotype control did not bind MDA-MB-231 cells (no binding, nb).

As described in Example 11 and shown in Table 5, the binding affinity of anti-hTF antibodies was evaluated on TF from cynomolgus monkey (Macaca fascicularis). The protein sequences of Macaca fascicularis TF and Macaca mulatta TF are identical. The binding of the TF-specific antibodies to cynomolgus monkey was confirmed using the Macaca mulatta RF/6A cell line as shown in Table 42. All tested anti-hTF antibodies exhibit high affinity to TF-positive Macaca mulatta RF/6A cells with an EC₅₀ ranging from about 1.28 nM to about 0.17 nM. The ability of the anti-TF antibodies to bind to cynomolgus monkey is advantageous for toxicology studies of these antibodies with nonhuman primate models.

TABLE 42
Binding of anti-TF antibodies to Macaca mulatta RF/6A cells

| Ab | RF/6A EC50 (nM) | RF/6A 95% CI |
|---|---|---|
| 1F | 0.17 | 0.14 to 0.21 |
| 25A | 0.43 | 0.37 to 0.50 |
| 25A3 | 0.27 | 0.24 to 0.30 |
| 25G1 | 0.27 | 0.23 to 0.32 |
| 29E | 0.53 | 0.46 to 0.61 |
| 39A | 0.27 | 0.23 to 0.32 |
| 43B1 | 0.47 | 0.40 to 0.55 |
| 43D7 | 0.41 | 0.35 to 0.49 |
| 43Ea | 0.92 | 0.83 to 1.01 |
| 54E | 1.28 | 1.16 to 1.41 |

### Example 24: Binding Assay to E. Coli-Derived TF

E. coli-derived TF was expressed as a fusion between the OmpA signal sequence and TF ECD-His6, and purified by affinity and anion exchange chromatography. The binding of anti-TF antibodies 1F, 25A, 25A3, 25G1, 29E, 39A, 43B1, 43D7, 43Ea, and 54E to Expi293- or E. coli-derived TF was determined by protein ELISA studies. Plates coated with Expi293- or E. coli-derived TF-His were incubated with increasing concentrations of antibodies. After incubation with an HRP-conjugated secondary antibody (Jackson Immunoresearch), luminescence data were obtained and used to calculate an EC₅₀ with 95% confidence intervals using Prism. The EC₅₀'s and 95% confidence intervals of the antibodies are listed in Table 43.

TABLE 43
Binding of anti-TF antibodies to Expi293- or E. coli-derived TF

| | Expi293- | Expi293- | | |
|---|---|---|---|---|
| | derived TF | derived TF | E.coli-derived | E.coli-derived |
| | protein EC50 | protein 95% | TF protein | TF protein |
| Ab | (nM) | CI | EC50 (nM) | 95% CI |
| 1F | 0.41 | 0.37 to 0.46 | 0.32 | 0.30 to 0.34 |
| 25A | 0.54 | 0.49 to 0.60 | 0.35 | 0.30 to 0.41 |
| 25A3 | 0.47 | 0.39 to 0.56 | 0.36 | 0.31 to 0.42 |
| 25G1 | 0.42 | 0.36 to 0.47 | 0.31 | 0.29 to 0.33 |
| 29E | 0.98 | 0.78 to 1.24 | 0.68 | 0.39 to 1.26 |
| 39A | 0.45 | 0.39 to 0.53 | 0.34 | 0.28 to 0.40 |
| 43B1 | 0.57 | 0.53 to 0.61 | 0.39 | 0.34 to 0.44 |
| 43D7 | 0.71 | 0.62 to 0.80 | 0.43 | 0.35 to 0.53 |
| 43Ea | 0.74 | 0.68 to 0.81 | 0.46 | 0.40 to 0.53 |
| 54E | 0.96 | 0.73 to 1.29 | 0.38 | 0.22 to 0.62 |

All tested anti-hTF antibodies exhibit high affinity to E. coli-derived TF with an EC₅₀ ranging from about 0.68 nM to about 0.31 nM, which is comparable to the binding affinity of the antibodies to Expi293-derived TF (about 0.98 nM to 0.41 nM). These results indicate that although the anti-TF antibodies were selected against glycosylated TF from a human cell line, the antibodies can bind to E. coli-derived TF with similar affinity when measured by protein ELISA.

### Example 25: Thrombin Generation Assay (TGA)

TGA assay was performed using the calibrated-automated-thrombogram (CAT) instrument manufactured and distributed by STAGO (Diagnostica Stago SAS, Asnieres sur Seine, France). See Samama et al., Thromb Res, 2012, 129:e77-82, which is incorporated by reference in its entirety. The test method design was equivalent to a standard CAT assay measurement, except that the plasma source was normal pooled plasma (NPP) collected in 11 mM citrate supplemented with 100 μg/mL of corn trypsin inhibitor (citrate/CTI). The anti-TF antibodies were titrated at 0, 10, 50 and 100 nM and mixed with NPP in citrate/CTI. Relipidated TF was added to a 96-well assay plate, followed by addition of the antibody/NPP mixture. After a 10-min incubation or directly after combining the relipidated TF with antibody/NPP, thrombin generation was initiated by the addition of calcium and the thrombin substrate. The STAGO software was used to report the following parameters: Peak IIa (highest thrombin concentration generated on the thrombin generation curve [nM]); Lag Time (time from assay start to the moment 10 nM of thrombin is formed [min]); ETP (endogenous thrombin potential, area under the curve [nM×min]); and ttPeak (time from assay start to Peak IIa [min]). Percent peak thrombin generation (% Peak IIa), percent endogenous thrombin potential (% ETP), and percent ttPeak (% ttPeak) in the presence of each antibody relative to a no-antibody plasma control on the same plate were also reported. As used herein, the term “thrombin generation assay” (TGA) refers to the TGA used in this example.

The Peak IIa, Lag Time, ETP, ttPeak, % Peak IIa, % ETP, and % ttPeak in the presence of each antibody selected from 1F, 25A, 25A3, 25G1, 29E, 39A, 43B1, 43D7, 43Ea, 54E, TF-011, 5G9, and 10H10 without antibody incubation prior to addition of calcium and thrombin substrate are shown in Table 44. The Peak IIa, Lag Time, ETP, ttPeak, % Peak IIa, % ETP, and % ttPeak in the presence of each antibody selected from 1F, 25A, 25A3, 25G1, 29E, 39A, 43B1, 43D7, 43Ea, 54E, TF-011, 5G9, and 10H10 with 10 min antibody incubation prior to addition of calcium and thrombin substrate are shown in Table 45. The thrombin generation curve in the presence of 100 nM anti-TF antibody without antibody pre-incubation are shown in international PCT application PCT/US2019/012427 and U.S. utility application Ser. No. 16/959,652, incorporated herein by reference in their entirety. The Peak thrombin concentration in the presence of titrations of anti-TF antibodies without antibody pre-incubation are shown in international PCT application PCT/US2019/012427 and U.S. utility application Ser. No. 16/959,652, incorporated herein by reference in their entirety.

As shown in international PCT application PCT/US2019/012427 and U.S. utility application Ser. No. 16/959,652 (incorporated herein by reference in their entirety), under the conditions without antibody pre-incubation, at the 100 nM antibody concentration, 1F, 29E, 39A, 54E diminished the peak IIa concentration by 92, 76, 91 and 70%, respectively. Similarly, 100 nM of 5G9 and TF-011 inhibited peak IIa concentration by 92% and 91 00 respectively. Severely reduced thrombin generation in the presence of the two highest concentrations of 1F, 39A, 5G9 and TF-011 hampered endogenous thrombin generation (ETP) calculations and increased time to Peak IIa/thrombin generation (ttPeak) by at least 284% and 353% at 50 nM and 100 nM respectively. In contrast, antibodies from group 25 did not impact the peak IIa concentration or ttPeak by more than 9%. Group 43 antibodies and 10H10 exhibited mild interference with the peak IIa concentration: 100 nM of 43B1, 43D7, 43Ea and 10H10 reduced the peak IIa concentration by 33, 44, 13 and 34%, respectively. In addition, 100 nM of 43B1, 43D7 and 10H10 showed at least a 29% increase in ttPeak. However, the observed decline in peak IIa concentration and delayed ttPeak for group 43 antibodies and 10H10 did not result in more than a 10% decline in the ETP.

Similar results are shown in Table 45 under the conditions with 10 min antibody pre-incubation. At the 100 nM antibody concentration, 1F, 29E, 39A, 54E diminished the peak IIa concentration by 93, 72, 93 and 87%, respectively. Similarly, 100 nM of 5G9 and TF-011 inhibited peak IIa concentration by 92% and 91%, respectively. Severely reduced thrombin generation in the presence of the two highest concentrations of 1F, 39A, 54E and TF-011 and all tested concentrations of 5G9 hampered endogenous thrombin generation (ETP) calculations and increased time to Peak IIa/thrombin generation (ttPeak) by at least 303% and 371% at 50 nM and 100 nM respectively. In contrast, antibodies from group 25 did not decrease the peak IIa concentration or increase ttPeak. Group 43 antibodies and 10H10 exhibited mild interference with the peak IIa concentration: 100 nM of 43B1, 43D7, 43Ea and 10H10 reduced the peak IIa concentration by 41, 56, 13 and 48%, respectively. In addition, 100 nM of 43B1, 43D7 and 10H10 showed at least a 33% increase in ttPeak. However, the observed decline in peak a concentration and delayed ttPeak for group 43 antibodies and 10H10 did not result in more than an 11% decline in the ETP.

Overall, these results indicate that group 25 antibodies are completely inert in the penultimate step of the coagulation cascade when all three TGA parameters (ETP, Peak IIa concentration and ttPeak) are taken into consideration.

TABLE 44
Thrombin Generation Assay without Antibody Pre-Incubation

| | | | Peak IIa | | Lag Time | | ETP | | ttPeak | | | | |
|---|---|---|---|---|---|---|---|---|---|---|---|---|---|
| | | Ab conc. | [nM] | | [min] | | [nM · min] | | [min] | | % Peak | | |
| Plate | Sample | (nM) | (SD) | | (SD) | | (SD) | | (SD) | | IIa | % ETP | % ttPeak |
| 1 | 1F | 100 | 25 | (1) | 31 | (1) | * | | 41 | (0.7) | 8 | * | 419 |
| | | 50 | 31 | (0) | 25.6 | (0.3) | * | | 35.3 | (0.3) | 9 | * | 347 |
| | | 10 | 155 | (1) | 8.2 | (0.2) | 1738 | (25) | 14.9 | (0.2) | 47 | 86 | 89 |
| 1 | 25A | 100 | 317 | (6) | 5.2 | (0.2) | 2134 | (28) | 8.6 | (0.2) | 95 | 105 | 9 |
| | | 50 | 317 | (2) | 5.2 | (0.2) | 2122 | (30) | 8.6 | (0.2) | 95 | 105 | 9 |
| | | 10 | 322 | (2) | 5 | (0) | 2108 | (29) | 8.2 | (0.2) | 97 | 104 | 4 |
| 1 | 25A3 | 100 | 323 | (1) | 4.6 | (0.2) | 2031 | (19) | 7.9 | (0.2) | 97 | 100 | 0 |
| | | 50 | 328 | (2) | 4.7 | (0) | 2080 | (23) | 8 | (0) | 98 | 103 | 1 |
| | | 10 | 326 | (4) | 5.3 | (0) | 2152 | (14) | 8.4 | (0.2) | 98 | 106 | 6 |
| 1 | 25G1 | 100 | 340 | (3) | 5.3 | (0) | 2160 | (27) | 8.3 | (0) | 102 | 107 | 5 |
| | | 50 | 346 | (6) | 5.1 | (0.2) | 2221 | (40) | 8.2 | (0.2) | 104 | 110 | 4 |
| | | 10 | 337 | (1) | 4.7 | (0) | 2061 | (34) | 7.8 | (0.2) | 101 | 102 | 1 |
| 1 | 29E | 100 | 81 | (0) | 17.1 | (0.2) | 1257 | (18) | 26.2 | (0.2) | 24 | 62 | 232 |
| | | 50 | 95 | (1) | 14.1 | (0.2) | 1365 | (26) | 22.6 | (0.4) | 29 | 67 | 186 |
| | | 10 | 235 | (3) | 7 | (0) | 1926 | (9) | 11.7 | (0) | 71 | 95 | 48 |
| 1 | Isotype | 100 | 326 | (3) | 5.3 | (0) | 2132 | (13) | 8.6 | (0.2) | 98 | 105 | 9 |
| | | 50 | 331 | (3) | 5.3 | (0) | 2177 | (19) | 8.3 | (0) | 99 | 108 | 5 |
| | | 10 | 328 | (4) | 5.3 | (0) | 2129 | (26) | 8.4 | (0.2) | 98 | 105 | 6 |
| 1 | TF-011 | 100 | 30 | (1) | 26 | (0.3) | * | | 35.8 | (0.2) | 9 | * | 353 |
| | | 50 | 39 | (3) | 21.3 | (0.5) | * | | 30.3 | (1.1) | 12 | * | 284 |
| | | 10 | 156 | (7) | 8 | (0) | 1714 | (41) | 14.7 | (0.5) | 47 | 85 | 86 |
| 1 | 5G9 | 100 | 27 | (1) | 29.9 | (0.4) | * | | 39.6 | (0.4) | 8 | * | 401 |
| | | 50 | 28 | (0) | 25.1 | (0.4) | * | | 34.6 | (0.2) | 8 | * | 338 |
| | | 10 | 79 | (1) | 10.4 | (0.2) | 1176 | (16) | 18.6 | (0.2) | 24 | 58 | 135 |
| 1 | 10H10 | 100 | 221 | (4) | 5.2 | (0.2) | 1945 | (37) | 10.2 | (0.2) | 66 | 96 | 29 |
| | | 50 | 248 | (3) | 5.2 | (0.2) | 1978 | (32) | 9.8 | (0.3) | 74 | 98 | 24 |
| | | 10 | 310 | (2) | 5.2 | (0.2) | 2036 | (33) | 8.6 | (0.2) | 93 | 101 | 9 |
| 1 | Plasma | NA | 333 | (0) | 4.7 | (0) | 2023 | (30) | 7.9 | (0.2) | 100 | 100 | 0 |
| | ctrl. | | | | | | | | | | | | |
| 2 | 39A | 100 | 29 | (0) | 34.7 | (0) | * | | 44.6 | (0.2) | 9 | * | 465 |
| | | 50 | 36 | (1) | 29.8 | (0.7) | * | | 39.3 | (0.7) | 11 | * | 397 |
| | | 10 | 122 | (3) | 10.8 | (0.3) | 1694 | (57) | 18.6 | (0.2) | 37 | 84 | 135 |
| 2 | 43B1 | 100 | 238 | (4) | 5.3 | (0) | 2300 | (32) | 10.8 | (0.2) | 67 | 99 | 37 |
| | | 50 | 258 | (5) | 5.2 | (0.2) | 2301 | (29) | 10.2 | (0.2) | 72 | 99 | 29 |
| | | 10 | 317 | (1) | 5 | (0) | 2341 | (34) | 8.6 | (0.2) | 89 | 101 | 9 |
| 2 | 43D7 | 100 | 199 | (6) | 5.1 | (0.2) | 2124 | (27) | 11.2 | (0.2) | 56 | 91 | 42 |
| | | 50 | 234 | (1) | 5 | (0) | 2190 | (15) | 10.3 | (0) | 66 | 94 | 30 |
| | | 10 | 312 | (3) | 5 | (0) | 2343 | (49) | 8.9 | (0.2) | 88 | 101 | 13 |
| 2 | 43Ea | 100 | 308 | (2) | 5 | (0) | 2349 | (9) | 9 | (0) | 87 | 101 | 14 |
| | | 50 | 316 | (3) | 5 | (0) | 2430 | (69) | 8.7 | (0) | 89 | 105 | 10 |
| | | 10 | 337 | (4) | 5 | (0) | 2416 | (82) | 8.3 | (0) | 95 | 104 | 5 |
| 2 | 54E | 100 | 108 | (3) | 12.2 | (0.2) | 1589 | (13) | 20.2 | (0.2) | 30 | 68 | 156 |
| | | 50 | 191 | (2) | 8 | (0) | 2109 | (51) | 14.3 | (0) | 54 | 91 | 81 |
| | | 10 | 311 | (5) | 5 | (0) | 2275 | (41) | 8.8 | (0.2) | 87 | 98 | 11 |
| 2 | Isotype | 100 | 351 | (2) | 4.7 | (0) | 2304 | (14) | 7.9 | (0.2) | 99 | 99 | 0 |
| | | 50 | 353 | (1) | 5 | (0) | 2391 | (29) | 8.2 | (0.2) | 99 | 103 | 4 |
| | | 10 | 348 | (1) | 5 | (0) | 2367 | (9) | 8.3 | (0) | 98 | 102 | 5 |
| 2 | Plasma | NA | 356 | (1) | 4.9 | (0.2) | 2323 | (76) | 8.11 | (0.3) | 100 | 100 | 3 |
| | ctrl. | | | | | | | | | | | | |

* Groups with “No Tail Found” Errors when the software cannot calculate the ETP.

TABLE 45
Thrombin Generation Assay with 10 min Antibody Pre-Incubation

| | | | Peak IIa | | Lag Time | | ETP | | ttPeak | | | | |
|---|---|---|---|---|---|---|---|---|---|---|---|---|---|
| | | Ab conc. | [nM] | | [min] | | [nM · min] | | [min] | | % Peak | % | % |
| Plate | Sample | (nM) | (SD) | | (SD) | | (SD) | | (SD) | | IIa | ETP | ttPeak |
| 1 | 1F | 100 | 20 | (1) | 29.5 | (0.2) | * | | 40.8 | (0.6) | 7 | * | 483 |
| | | 50 | 23 | (0) | 26.5 | (0.7) | * | | 37.3 | (0.4) | 8 | * | 433 |
| | | 10 | 44 | (2) | 13.8 | (0.5) | 742 | (23) | 22.4 | (0.4) | 16 | 41 | 220 |
| 1 | 25A | 100 | 291 | (3) | 3.3 | (0.1) | 1964 | (36) | 6.7 | (0.1) | 106 | 108 | −4 |
| | | 50 | 290 | (0) | 3.3 | (0.1) | 1972 | (22) | 6.8 | (0) | 106 | 108 | −3 |
| | | 10 | 284 | (1) | 3.3 | (0.1) | 1899 | (21) | 6.8 | (0) | 104 | 104 | −3 |
| 1 | 25A3 | 100 | 290 | (3) | 3.1 | (0) | 1893 | (28) | 6.4 | (0) | 106 | 104 | −9 |
| | | 50 | 284 | (4) | 3.1 | (0) | 1875 | (16) | 6.4 | (0) | 104 | 103 | −9 |
| | | 10 | 288 | (3) | 3.1 | (0) | 1901 | (26) | 6.4 | (0) | 105 | 105 | −9 |
| 1 | 25G1 | 100 | 311 | (3) | 3.1 | (0) | 1954 | (20) | 6.3 | (0.1) | 114 | 107 | −10 |
| | | 50 | 311 | (1) | 3.1 | (0) | 1951 | (22) | 6.1 | (0) | 114 | 107 | −13 |
| | | 10 | 302 | (3) | 3.1 | (0) | 1877 | (33) | 6.1 | (0) | 110 | 103 | −13 |
| 1 | 29E | 100 | 76 | (1) | 14.7 | (0.1) | 1201 | (24) | 24.3 | (0.3) | 28 | 66 | 247 |
| | | 50 | 83 | (1) | 14.1 | (0) | 1300 | (17) | 23.6 | (0.1) | 30 | 72 | 237 |
| | | 10 | 98 | (1) | 9.4 | (0) | 1408 | (11) | 18.1 | (0) | 36 | 77 | 159 |
| 1 | Isotype | 100 | 288 | (2) | 3.4 | (0) | 1922 | (28) | 6.8 | (0) | 105 | 106 | −3 |
| | | 50 | 292 | (2) | 3.4 | (0) | 1921 | (25) | 6.8 | (0) | 107 | 106 | −3 |
| | | 10 | 290 | (3) | 3.4 | (0) | 1926 | (38) | 6.8 | (0) | 106 | 106 | −3 |
| 1 | TF-011 | 100 | 26 | (0) | 23.8 | (1.1) | * | | 34.2 | (0.9) | 9 | * | 389 |
| | | 50 | 27 | (1) | 22.4 | (0.1) | * | | 33 | (0.1) | 10 | * | 371 |
| | | 10 | 46 | (3) | 13.5 | (0.5) | 792 | (55) | 22.5 | (0.2) | 17 | 44 | 221 |
| 1 | 5G9 | 100 | 22 | (0) | 26.7 | (0.3) | * | | 37.5 | (0.5) | 8 | * | 436 |
| | | 50 | 23 | (3) | 23.6 | (2.2) | * | | 34 | (2.4) | 8 | * | 386 |
| | | 10 | 30 | (1) | 19.3 | (0.4) | * | | 29 | (0.8) | 11 | * | 314 |
| 1 | 10H10 | 100 | 169 | (3) | 3.4 | (0) | 1795 | (36) | 9.3 | (0.1) | 62 | 99 | 33 |
| | | 50 | 175 | (4) | 3.4 | (0) | 1754 | (20) | 9.2 | (0.1) | 64 | 96 | 31 |
| | | 10 | 235 | (8) | 3.4 | (0) | 1807 | (42) | 7.8 | (0) | 86 | 99 | 11 |
| 1 | Plasma | NA | 274 | (1) | 3.4 | (0) | 1818 | (24) | 7 | (0.1) | 100 | 100 | 0 |
| | ctrl. | | | | | | | | | | | | |
| 2 | 39A | 100 | 19 | (1) | 33.6 | (0.7) | * | | 44.6 | (0.9) | 7 | * | 537 |
| | | 50 | 22 | (0) | 30.7 | (0.1) | * | | 41.4 | (0.1) | 8 | * | 491 |
| | | 10 | 36 | (1) | 19.6 | (0.7) | * | | 29.3 | (0.8) | 13 | 0 | 319 |
| 2 | 43B1 | 100 | 167 | (0) | 4 | (0) | 1806 | (15) | 9.8 | (0.1) | 59 | 98 | 40 |
| | | 50 | 174 | (1) | 3.8 | (0.1) | 1831 | (22) | 9.6 | (0) | 62 | 99 | 37 |
| | | 10 | 222 | (5) | 3.7 | (0.1) | 1841 | (37) | 8.3 | (0) | 79 | 100 | 19 |
| 2 | 43D7 | 100 | 123 | (2) | 4 | (0) | 1673 | (27) | 11.5 | (0.1) | 44 | 91 | 64 |
| | | 50 | 122 | (1) | 3.7 | (0.1) | 1639 | (29) | 11.3 | (0) | 43 | 89 | 61 |
| | | 10 | 194 | (5) | 4 | (0) | 1796 | (35) | 8.8 | (0.1) | 69 | 97 | 26 |
| 2 | 43Ea | 100 | 244 | (2) | 3.5 | (0.1) | 1857 | (42) | 7.5 | (0.1) | 87 | 101 | 7 |
| | | 50 | 245 | (0) | 3.6 | (0) | 1851 | (29) | 7.6 | (0) | 87 | 100 | 9 |
| | | 10 | 262 | (1) | 3.6 | (0) | 1877 | (15) | 7.3 | (0) | 93 | 102 | 4 |
| 2 | 54E | 100 | 37 | (1) | 22.3 | (0.2) | * | | 33 | (0.5) | 13 | * | 371 |
| | | 50 | 44 | (1) | 18.3 | (0.4) | * | | 28.2 | (1) | 16 | * | 303 |
| | | 10 | 121 | (4) | 6.5 | (0.1) | 1523 | (20) | 13.7 | (0.3) | 43 | 83 | 96 |
| 2 | Isotype | 100 | 275 | (2) | 3.6 | (0) | 1862 | (23) | 7.3 | (0) | 98 | 101 | 4 |
| | | 50 | 284 | (0) | 3.6 | (0) | 1899 | (15) | 7.2 | (0.1) | 101 | 103 | 3 |
| | | 10 | 281 | (3) | 3.6 | (0) | 1877 | (13) | 7.3 | (0) | 100 | 102 | 4 |
| 2 | Plasma | NA | 282 | (2) | 3.8 | (0.1) | 1845 | (22) | 7.3 | (0) | 100 | 100 | 4 |
| | ctrl. | | | | | | | | | | | | |

* Groups with “No Tail Found” Errors when the software cannot calculate the ETP.

### Example 26: FXa Conversion Assay and FVIIa Competition Assay with Previously Described Anti-TF Antibodies

The previously described TF-specific antibodies TF-011, 5G9 and 10H10 (Breij et al., Cancer Res, 2014, 74:1214-1226; Versteeg et al., Blood, 2008,111:190-199; each of which is incorporated by reference in its entirety) were tested in FXa conversion assay and FVIIa competition assay.

To evaluate the ability of TF:FVIIa to convert FX into FXa in the presence of human antibodies against TF, a cell-based FX conversion assay was conducted as described in Larsen et al., J Biol Chem, 2010, 285:19959-19966, which is incorporated by reference in its entirety. Briefly, 5×10⁴ MDA-MB-231 cells (ATCC, Manassas, VA, USA) were plated into tissue culture-treated black 96-well plates (Greiner Bio-One, Monroe, NC, USA) and cultured overnight. After removal of the cell culture media and addition of a final concentration of 200 nM of FX in a HEPES buffer with 1.5 mM CaCl₂), cells were incubated with a titration of the antibodies for 15 min at 37° C. Upon reconstitution of the binary TF:FVIIa complex with a final concentration of 20 nM of FVIIa, cells were incubated for 5 min at 37° C. After quenching the reaction with ethylenediaminetetraacetic acid (EDTA) in a black 94-well plate, generated FXa was measured with 50 pM of SN-7 6-amino-1-naphthalenesulfonamide-based fluorogenic substrate (Haematologic Technologies, Essex Junction, VT, USA) on an Envision plate reader equipped with an Umbelliferone 355 excitation filter, an Umbelliferone 460 emission filter, and a LANCE/DELFIA top mirror (Perkin Elmer, Waltham, MA, USA). FXa conversion percentages (% FXa) in the presence of an anti-TF antibody titration relative to a no antibody control are shown in international PCT application PCT/US2019/012427 and U.S. utility application Ser. No. 16/959,652, incorporated herein by reference in their entirety.

To evaluate competition between FVIIa and the human antibodies against TF, TF-positive MDA-MB-231 cells (ATCC, Manassas, VA, USA) were first incubated for 1 hr on ice with a titration of the human antibodies against TF or an isotype control. Subsequently, FVII-Fc conjugated to Alexa488 was added to the antibody-cell mixture at a final concentration of 20 nM. After another 1 hr incubation on ice, cells were washed, stained with a viability dye, and analyzed by flow cytometry. The Alexa488 fluorescence data from viable cells was summarized using median fluorescence intensity (MFI). FVII-Fc binding was summarized with % FVII-Fc binding=[MFI_{antibody labeled cells}—MFI_{unstained} cells]/[MFI_{IgG1 control labeled cells}—MFI_{unstained} cells]. Percentage of FVIIa binding (% FVIIa) in the presence of an anti-TF antibody titration relative to an isotype control is shown in international PCT application PCT/US2019/012427 and U.S. utility application Ser. No. 16/959,652, incorporated herein by reference in their entirety.

As shown in international PCT application PCT/US2019/012427 and U.S. utility application Ser. No. 16/959,652 (incorporated herein by reference in their entirety), TF-011 and 5G9 inhibited FX conversion by 57-59% and 67-70% at concentrations of 25, 50, and 100 nM. 10H10 did not significantly inhibit FX conversion at these three concentraions.

As shown in international PCT application PCT/US2019/012427 and U.S. utility application Ser. No. 16/959,652 (incorporated herein by reference in their entirety), TF-011 effectively competed with FVII, whereas 5G9 and 10H10 showed less than 25% and 10% competition at the highest concentration of antibody, respectively.

These results indicate that 5G9 predominantly competes with substrate FX binding, resulting in the observed inhibition of FX conversion and thrombin generation. TF-011 inhibits thrombin generation by competing with FVIIa for binding to TF. However, 10H10 inhibits TF-FVIIa mediated signaling without substantially affecting binding of FVIIa to TF. These findings are consistent with previous observations described in Huang et al., J Mol Biol, 1998, 275:873-894; Ruf et al., Biochem J, 1991, 278:729-733; and Teplyakov et al., Cell Signal, 2017, 36:139-144; each of which is incorporated by reference in its entirety.

### Example 27: Antibody Competition Assay

Alexa Fluor antibodies were generated using Alexa Fluor 488 5-sulfo-dichlorophenol esters (ThermoFisher Scientific) following manufacturer's protocol. Excess Alexa Fluor dye was removed from the antibody dye conjugate preparations by gel filtration (ThermoFisher Scientific).

To evaluate competition between a first human antibody against TF and 25A3, TF-positive MDA-MB-231 cells (ATCC, Manassas, VA, USA) were first incubated for 1 hr on ice with a titration of the first human antibody against TF. Subsequently, a final concentration of 20 nM of 25A3 conjugated to Alexa488 was added to the antibody cell mixture. After another 1 hr incubation on ice, cells were washed, stained with a viability dye, and analyzed by flow cytometry. The Alexa488 fluorescence data from viable cells was summarized using median fluorescence intensity. 25A3 binding was summarized with % 25A3 binding=[MFI_{antibody labeled cells} —MFI_{unstained} cells]/[MFI_{IgG1 control labeled cells}—MFI_{unstained} cells].

To evaluate competition between a first human antibody against TF and 43D7, TF-positive MDA-MB-231 cells (ATCC, Manassas, VA, USA) were first incubated for 1 hr on ice with a titration of the first human antibody against TF. Subsequently, a final concentration of 20 nM of 43D7 conjugated to Alexa488 was added to the antibody cell mixture. After another 1 hr incubation on ice, cells were washed, stained with a viability dye, and analyzed by flow cytometry. The Alexa488 fluorescence data from viable cells was summarized using median fluorescence intensity. 43D7 binding was summarized with % 43D7 binding=[MFI_{antibody labeled cells} —MFI_{unstained} cells]/[MFI_{IgG1 control labeled cells}−MFI_{unstained} cells].

To evaluate competition between a first human antibody against TF and 39A, TF-positive MDA-MB-231 cells (ATCC, Manassas, VA, USA) were first incubated for 1 hr on ice with a titration of the first human antibody against TF. Subsequently, a final concentration of 20 nM of 39A conjugated to Alexa488 was added to the antibody cell mixture. After another 1 hr incubation on ice, cells were washed, stained with a viability dye, and analyzed by flow cytometry. The Alexa488 fluorescence data from viable cells was summarized using median fluorescence intensity. 39A binding was summarized with % 39A binding=[MFI_{antibody labeled cells}−MFI_{unstained} cells]/[MFI_{IgG1 control labeled cells}−MFI_{unstained} cells].

% 25A3 binding, % 43D7 binding, and % 39A binding are shown in international PCT application PCT/US2019/012427 and U.S. utility application Ser. No. 16/959,652, incorporated herein by reference in their entirety. Antibodies from groups 25 and 43, 5G9, and 10H10 reduced % 25A3 binding and % 43D7 binding and did not reduce % 39A binding. Antibodies from groups 1, 29, 39, and 54, and TF-011 reduced % 39A binding and did not reduce % 25A3 binding and % 43D7 binding.

While the antibody competition assay results indicate that groups 25 and 43 antibodies, 5G9, and 10H10 may bind to the same or an overlapping epitope of human TF or may affect the TF binding of each other through an allosteric mechanism, the chimeric TF construct mapping experiments as described elsewhere in this disclosure demonstrate that group 25 antibodies, group 43 antibodies, 5G9 and 10H10 bind distinct epitopes. In addition, while the antibody competition assay results indicate that antibodies of groups 1, 29, 39, and 54, and TF-011 may bind to the same or an overlapping epitope of human TF or may affect the TF binding of each other through an allosteric mechanism, the chimeric TF construct mapping experiments as described elsewhere in this disclosure demonstrate that the antibodies of groups 29, 39 and 54 bind epitopes distinct from TF-011's epitope.

### Example 28: Anti-TF Antibody Internalization

To evaluate internalization of the anti-TF antibodies, a cytotoxicity assay was conducted as described in Liao-Chan et al., PLoS One, 2015, 10:e0124708, which is incorporated by reference in its entirety. Briefly, cells were plated in 384-well plates (Greiner Bio-One, Monroe, NC, USA) at 4×10³ cells per well in 40 μl of media. Antibodies and an anti-human Fc Fab conjugated to the tubulin inhibitor mono-methyl auristatin F (MMAF) (Moradec, San Diego, CA, USA) were serially diluted starting at 5 and 30 nM, respectively.

The anti-human Fc Fab conjugated to MMAF consisted of a polyclonal antibody specific to the Fc region of human IgGs with a DAR of 1.2 to 1.5. Plates were incubated for 3 days, followed by lysis in CellTiter-Glo (CTG) assay reagent (Promega, Madison, WI, USA). CTG luminescence was measured on an Envision plate reader and the mean and standard deviation of 4 replicates graphed in Prism (GraphPad, La Jolla, CA, USA). For each anti-TF antibody, the IC₅₀ and its associated 95% confidence interval were calculated in Prism using a 4-parameter binding model. The cell viability results after incubation with anti-TF antibodies and anti-TF antibody Fab:MMAF complexes are shown in international PCT application PCT/US2019/012427 and U.S. utility application Ser. No. 16/959,652, incorporated herein by reference in their entirety. The 95% confidence intervals for the IC₅₀ values are shown in Table 46.

Internalization of the anti-TF antibodies was also evaluated by a quantitative assay based on internalized fluorescence and quenched surface-fluorescence. Cell surface fluorescence quenching was assessed as described in Liao-Chan et al., PLoS One, 2015,10:e0124708. Briefly, 1.2×10⁵ MDA-MB-231 cells were pre-incubated with 100 nM of A488-conjugated antibodies in media for 2 hr on ice. After 2 washes, cells were resuspended in cold media and pulsed for up to 4 hr at 37° C. Cells were rapidly chilled and incubated with or without 300 nM of anti-A488 antibody (clone 19A) for 30 min on ice. After 2 washes, dead cells were labeled with DAPI and samples were analyzed on a Novocyte flow cytometer (ACEA Biosciences). The median fluorescence intensities (MFIs) at each anti-A488 mAb concentration were normalized against the isotype control to obtain a normalized MFI percentage. Internalized fluorescence was calculated from quenched and non-quenched sample data by correcting for incomplete surface quenching: 1−(N₁−Q₁)/(N₁−(N₁Q₀/N₀)) with N₁=unquenched MFI at each time point (ti); Q₁=Quenched MFI at t₁; Q₀=Quenched MFI for the sample kept on ice (t₀); N₀=Unquenched MFI at to. Percent internalization of anti-TF antibodies conjugated to A488 is shown in international PCT application PCT/US2019/012427 and U.S. utility application Ser. No. 16/959,652, incorporated herein by reference in their entirety.

Because Fab:MMAF binds the Fc region of the TF-specific antibodies, cellular uptake of these complexes can trigger cell death. While the TF-specific antibodies alone had no impact on cell viability in three-day cultures of TF-positive A431 cells, the TF-specific antibodies in complex with Fab:MMAF showed dose-dependent cell killing with IC₅₀ values ranging between 0.07 and 0.14 nM. (See international PCT application PCT/US2019/012427 and U.S. utility application Ser. No. 16/959,652, incorporated herein by reference in their entirety).

Cellular uptake was corroborated with fluorescently labeled TF-specific antibodies. In a quantitative assay based on internalized fluorescence and quenched surface-fluorescence, the TF-specific antibodies showed between 28 and 37% internalization after a 4 h incubation. (See international PCT application PCT/US2019/012427 and U.S. utility application Ser. No. 16/959,652, incorporated herein by reference in their entirety).

These results indicate that the tested anti-TF antibodies can medicate internalization and toxin delivery into TF-positive cells.

TABLE 46
ADC Data With Ranking (Continuous Incubation). *The referenced figures are shown in international PCT application
PCT/US2019/012427 and US utility application number 16/959,652, incorporated herein by reference in their entirety.

| | | Cell line | | | | | | | | | | | | |
|---|---|---|---|---|---|---|---|---|---|---|---|---|---|---|
| | | A431 | | | A431 | | | MDA-MB-231 | | | HPAF-II | | | |
| | | ADC format | | | | | | | | | | | | |
| | | Secondary ADC | | | Primary ADC | | | Primary ADC | | | Primary ADC | | | |
| | | Treatment | | | | | | | | | | | | |
| | | Continuous | | | Continuous | | | Continuous | | | Continuous | | | |
| | | FIG. | | | | | | | | | | | | |
| | | FIG. 18B | | | FIG. 20A | | | FIG. 22D | | | FIG. 22E | | | |
| | | Binding data | | | | | | | | | | | | Continuous |
| | | IC₅₀ | 95% | | IC₅₀ | 95% | | IC₅₀ | 95% | | IC₅₀ | 95% | | Primary |
| | | (nM) | CI | rank | (nM) | CI | rank | (nM) | CI | rank | (nM) | CI | rank | ADC RANK |
| Antibody: | 1F | 0.07 | 0.06 to | Not | Not | | | Not | | | Not | | | Not |
| | | | 0.07 | included | tested | | | tested | | | tested | | | included |
| | 25A | 0.11 | 0.10 to | 6 | 0.09 | 0.08 to | 7 | 0.14 | 0.12 to | 7 | 0.06 | 0.05 to | 8 | 7 |
| | | | 0.11 | | | 0.09 | | | 0.16 | | | 0.07 | | |
| | 25A3 | 0.09 | 0.08 to | 3 | 0.07 | 0.07 to | 5 | 0.11 | 0.10 to | 4 | 0.05 | 0.04 to | 5 | 4 |
| | | | 0.09 | | | 0.08 | | | 0.12 | | | 0.05 | | |
| | 25G1 | 0.08 | 0.07 to | 1 | 0.06 | 0.06 to | 3 | 0.09 | 0.08 to | 3 | 0.04 | 0.04 to | 3 | 3 |
| | | | 0.08 | | | 0.06 | | | 0.10 | | | 0.05 | | |
| | 29E | 0.10 | 0.09 to | 4 | 0.06 | 0.05 to | 2 | 0.07 | 0.07 to | 2 | 0.04 | 0.04 to | 2 | 2 |
| | | | 0.10 | | | 0.06 | | | 0.08 | | | 0.05 | | |
| | 39A | 0.08 | 0.08 to | 2 | 0.05 | 0.05 to | 1 | 0.05 | 0.05 to | 1 | 0.04 | 0.03 to | 1 | 1 |
| | | | 0.09 | | | 0.05 | | | 0.05 | | | 0.05 | | |
| | 43B1 | 0.12 | 0.11 to | 7 | 0.08 | 0.08 to | 6 | 0.14 | 0.13 to | 5 | 0.05 | 0.04 to | 4 | 5 |
| | | | 0.13 | | | 0.08 | | | 0.15 | | | 0.06 | | |
| | 43D7 | 0.10 | 0.10 to | 5 | 0.06 | 0.06 to | 4 | 0.14 | 0.12 to | 6 | 0.05 | 0.05 to | 6 | 6 |
| | | | 0.10 | | | 0.07 | | | 0.16 | | | 0.06 | | |
| | 43Ea | 0.13 | 0.13 to | 8 | 0.09 | 0.09 to | 8 | 0.15 | 0.13 to | 8 | 0.06 | 0.05 to | 7 | 8 |
| | | | 0.14 | | | 0.10 | | | 0.17 | | | 0.06 | | |

| | | | | | | | | | |
|---|---|---|---|---|---|---|---|---|---|
| 54E | 0.11 | 0.11 to | Not | 0.07 | 0.07 to | Not | Not | Not | Not |
| | | 0.12 | included | | 0.07 | included | tested | tested | included |

| Isotype | Not | Not | | Not | Not | Not |
|---|---|---|---|---|---|---|
| | applicable | applicable | | applicable | applicable | included |
| TF-011 | Not | 0.05 | 0.05 to 0.05 | Not | Not | Not |
| | tested | | | tested | tested | included |

TABLE 47
ADC Data With Ranking (4 h Incubation). *The referenced figures are shown
in international PCT application PCT/US2019/012427 and US utility application
number 16/959,652, incorporated herein by reference in their entirety.

| | | Cell line | | | | | | |
|---|---|---|---|---|---|---|---|---|
| | | A431 | | | A431 | | | |
| | | ADC format | | | | | | |
| | | Primary ADC | | | Primary ADC | | | |
| | | Treatment | | | | | | |
| | | 4 hr, followed by washout | | | 4 hr, followed by washout | | | |
| | | FIG. | | | | | | |
| | | FIG. 20B | | | FIG. 21A | | | |
| | | Measurement | | | | | | 4 hr |
| | | IC₅₀ | 95% | | IC₅₀ | 95% | | Primary |
| | | (nM) | CI | rank | (nM) | CI | rank | ADC RANK |
| Antibody: | 1F | Not | | | Not | | | Not |
| | | tested | | | tested | | | included |
| | 25A | 0.35 | 0.32 to 0.39 | 6 | 0.18 | 0.17 to 0.19 | 6 | 6 |
| | 25A3 | 0.19 | 0.17 to 0.21 | 3 | 0.12 | 0.11 to 0.12 | 3 | 3 |
| | 25G1 | 0.19 | 0.17 to 0.20 | 2 | 0.10 | 0.09 to 0.10 | 2 | 2 |
| | 29E | 0.20 | 0.18 to 0.21 | 4 | 0.13 | 0.12 to 0.14 | 4 | 4 |
| | 39A | 0.12 | 0.11 to 0.13 | 1 | 0.09 | 0.09 to 0.10 | 1 | 1 |
| | 43B1 | 0.36 | 0.32 to 0.41 | 7 | 0.19 | 0.17 to 0.20 | 7 | 7 |
| | 43D7 | 0.28 | 0.25 to 0.30 | 5 | 0.14 | 0.13 to 0.15 | 5 | 5 |
| | 43Ea | 0.43 | 0.39 to 0.48 | 8 | 0.24 | 0.22 to 0.25 | 8 | 8 |
| | 54E | 0.26 | 0.24 to 0.29 | | 0.20 | 0.18 to 0.22 | | Not |
| | | | | | | | | included |
| | Isotype | Not | | | Not | | | Not |
| | | applicable | | | applicable | | | included |
| | TF-011 | 0.17 | 0.16 to 0.18 | | 0.09 | 0.09 to 0.10 | | Not |
| | | | | | | | | included |

### Example 29: Cell-Based Binding Assay of Antibody-Drug Conjugates (ADCs)

To evaluate the cell binding properties of ADCs, binding of anti-TF antibodies and anti-TF ADCs to endogenous human TF expressing HCT116 cells was assessed as previously described in Liao-Chan et al., PLoS One, 2015, 10:e0124708, which is incorporated by reference in its entirety. Briefly, 1.2×10⁵ cells collected with Cellstripper (Mediatech, Manassas, VA, USA) were incubated with a twelve-point 1:3 dilution titration of anti-human TF antibody or ADC starting at 100 nM for 2 hr on ice. After 2 washes, cells labeled with antibody or ADC were incubated for 30 min on ice with 150 nM of Goat Phycoerythrin (PE) F(ab′)₂ fragment goat anti-human IgG, Fcγ fragment specific (Jackson ImmunoResearch, West Grove, PA, USA) or FITC-labeled F(ab′)₂ fragment goat anti-human kappa (SouthernBiotech, Birmingham, AL, USA), respectively. After 2 washes, dead cells were labeled with TO-PRO-3 Iodide (ThermoFisher Scientific) and samples were analyzed on a CytoFLEX flow cytometer (Beckman Coulter, Brea, CA, USA) or Novocyte flow cytometer (ACEA Biosciences, San Diego, CA, USA). The median fluorescence intensities (MFIs) at each dilution were plotted and cell EC₅₀'s were derived using a 4-parameter binding model in Prism (GraphPad, La Jolla, CA, USA). The binding curves of anti-TF antibodies and anti-TF ADCs are shown in international PCT application PCT/US2019/012427 and U.S. utility application Ser. No. 16/959,652, incorporated herein by reference in their entirety. The reportable cell EC₅₀'s and their 95% confidence intervals of the anti-TF antibodies and ADCs are shown in international PCT application PCT/US2019/012427 and U.S. utility application Ser. No. 16/959,652, incorporated herein by reference in their entirety.

As shown in international PCT application PCT/US2019/012427 and U.S. utility application Ser. No. 16/959,652, incorporated herein by reference in their entirety, the cell binding properties of TF-specific ADCs are comparable to the cell binding properties of TF-specific antibodies, which indicates that the conjugation process of ADC did not alter the cell-binding properties of the TF-specific antibody moiety of the ADC.

### Example 30: Cytotoxicity Assays of Antibody-Drug Conjugates (ADCs)

To evaluate ADC cytotoxicity, A431 cells were plated in 384-well plates (Greiner Bio-One). Anti-TF antibodies conjugated to MC-vc-PAB-MMAE were serially diluted as shown. The TF-specific ADCs were added to A431 cells, with either a 72 h incubation or a 4 h incubation followed by removal of excess ADC and culture for another 68 h. A431 cells were lysed in CTG assay reagent after treatment. CTG luminescence was measured and the mean and standard deviation of 4 replicates graphed in Prism. For each ADC, the IC₅₀ and its associated 95% confidence interval were calculated in Prism using a 4-parameter binding model.

The cell viability after titrations of anti-TF ADCs with a continuous 72 h incubation is shown in international PCT application PCT/US2019/012427 and U.S. utility application Ser. No. 16/959,652, incorporated herein by reference in their entirety. The cell viability after titrations of anti-TF ADCs with a 4 h incubation followed by removal of excess ADC and culture for another 68 h is shown in international PCT application PCT/US2019/012427 and U.S. utility application Ser. No. 16/959,652, incorporated herein by reference in their entirety. The reportable IC₅₀ values of ADCs under both the continuous treatment and the pulse treatment are shown in international PCT application PCT/US2019/012427 and U.S. utility application Ser. No. 16/959,652, incorporated herein by reference in their entirety. The 95% confidence intervals for the IC₅₀'s of the continuous treatment and the pulse treatment are listed in Table 46 and Table 47 respectively.

Both treatments resulted in efficacious cell killing, with a 2.4 to 4.7-fold increase in IC₅₀ when excess ADC was removed from the culture after the 4 h incubation compared to the 72 h incubation. Removal of excess 25A3 and 39A ADC had the smallest impact on IC₅₀, with a 2.7 and 2.4-fold increase from 0.07 and 0.05 nM, respectively.

These results indicate that similar to the TF-specific antibodies, the TF-specific ADCs undergo substantial cellular internalization.

### Example 31: Cytotoxicity Assays in the Presence of FVIIa

To understand whether FVIIa interfered with the activity of the TF-specific ADC, we treated A431 cells for 4 h with the TF-specific ADCs (anti-TF antibodies conjugated to MC-vc-PAB-MMAE) in the absence or presence of FVIIa and measured cell viability 68 h later. A431 cells were pre-incubated for 30 min without or with 50 nM of FVIIa prior to the addition of an anti-TF ADC titration. Cell viability was determined by CTG assay. The mean and standard deviation of 4 replicates were graphed in Prism. For each ADC, the IC₅₀ were calculated in Prism using a 4-parameter binding model.

The cell viability after titrations of anti-TF ADCs in the absence or presence of FVIIa is shown in international PCT application PCT/US2019/012427 and U.S. utility application Ser. No. 16/959,652, incorporated herein by reference in their entirety. The reportable IC₅₀ values of ADCs in the absence or presence of FVIIa are shown in international PCT application PCT/US2019/012427 and U.S. utility application Ser. No. 16/959,652, incorporated herein by reference in their entirety.

While the ADCs that competed with FVIIa (29E, 39A, 54E and TF-011) were negatively affected by the presence of FVIIa by at least 2.3-fold, the ADCs that did not compete with FVIIa (group 25 and 43 antibodies) were equally efficacious in the absence or presence of FVIIa.

These results indicate that FVIIa does not interfere with the activity of anti-TF ADCs from groups 25 and 43.

### Example 32: Intracellular Microtubule Network in the Presence of Antibody-Drug Conjugates (ADCs)

Immunofluorescence of the intracellular microtubule network of cells was conducted to illustrate the mechanism of action of the ADC. See Theunissen et al., Methods Enzymol, 2006, 409:251-284. Briefly, A431 or HPAF-II cells were seeded onto 8-well poly-D-lysine treated slides (Corning Inc, Corning, NY, USA). One day later, the culture medium was replaced with medium containing ADC at 5 nM. After twenty hours of ADC exposure, the cells were fixed for 15 min at room temperature with 4% paraformaldehyde (ThermoFisher Scientific). After three washes with PBS, the cells were permeabilized for 1 h with PBS containing 0.3% Triton X-100 and 5% normal goat serum. Next, the microtubule networks were stained for 3 h with anti-tubulin (11H10) rabbit mAb (Alexa Fluor 488 conjugate) (Cell Signaling Technology, Danvers, MA, USA) in PBS containing 1% BSA and 0.3% Triton X-100. After three washes, ProLong Gold Antifade reagent with DAPI (ThermoFisher Scientific) was added to the cells and the slide was mounted for microscopy by using a 0.17 mm coverslip. Image acquisition was conducted on a DMi8 fluorescence microscope (Leica Microsystems, Buffalo Grove, IL, USA) equipped with a sCMOS camera. The Leica LAS X software was used to acquire a system-optimized Z-stack of 6 to 7 microns. A sharp two-dimensional image from this Z-stack was created automatically with the extended depth of field (EDF) image feature. Representative images of tubulin staining of A431 or HPAF-II cells are shown in international PCT application PCT/US2019/012427 and U.S. utility application Ser. No. 16/959,652, incorporated herein by reference in their entirety.

While the isotype control ADC did not affect the microtubule network, the 25A3 ADC disrupted the microtubule network effectively in both A431 and HPAF-II cells.

These results indicate the MMAE-based anti-TF ADCs induce cytotoxicity in TF-positive cancer cells through disruption of the intracellular microtubule network.

### Example 33: Cytotoxicity Assays and G₂/M Arrest in HUVECs

To evaluate TF copy number on the cell surface of human umbilical vein endothelial cells (HUVECs), 1.2×10⁵HUVECs were harvested and incubated with 133 nM of anti-human TF antibody 5G9 on a mouse IgG2a backbone for 2 hr on ice. After 2 washes, QIFIKIT beads (Agilent) and cells labeled with anti-TF antibody were incubated for 30 min on ice with 150 nM of Goat Phycoerythrin (PE) F(ab′)₂ fragment goat anti-mouse IgG, Fc-gamma fragment specific (Jackson ImmunoResearch). After 2 washes, dead cells were labeled with TO-PRO-3 Iodide (ThermoFisher Scientific) and samples were analyzed on a CytoFLEX flow cytometer (Beckman Coulter). After gating for single live cells, the MFI's were determined using FlowJo (Flowjo, Ashland, OR, USA). A standard curve using QIFIKIT beads was generated in Prism using a 5-parameter binding model to determine copy number. The lower limit of quantitation was 1.9×10³ antibody binding sites (also referred to as copy number) and the upper limit of quantitation was 8.0×10⁵ antibody binding sites.

In response to injury, inflammatory and angiogenic factors transiently increase expression of surface TF in the vasculature. See Holy et al., Adv Pharmacol, 2010, 59:259-592, which is incorporated by reference in its entirety. The transient upregulation of TF in cell culture was mimicked by treating HUVECs with a combination of inflammatory cytokines (5 ng/mL IL1-beta, 25 ng/mL TNF-alpha and 50 ng/mL VEGF). As shown in international PCT application PCT/US2019/012427 and U.S. utility application Ser. No. 16/959,652 (incorporated herein by reference in their entirety), surface TF levels increased from 2.4×10³ copies in the absence of inflammatory cytokines to 1.2×10⁴ copies after 6 h of cytokine treatment. The surface TF was ˜3-fold lower after 20 h of cytokine treatment relative to 6 h of treatment, which indicates that the cytokine-induced TF upregulation was transient.

For the ADC cytotoxicity assay, HUVEC cultures were seeded on half-area 96-well plates. The next day, the combination of inflammatory cytokines and a titration of ADCs was added to the cultures. Four days later viability of the cultures was assessed by lysis in CellTiter-Glo (CTG) assay reagent. As shown in international PCT application PCT/US2019/012427 and U.S. utility application Ser. No. 16/959,652, incorporated herein by reference in their entirety, the cell viability of inflammatory cytokine-treated HUVEC cultures was unaffected by the anti-TF ADCs, 25A-vc-MMAE and 43Ea-vc-MMAE. The results indicates that the inflammatory cytokine-treated endothelial cells are resistant to anti-TF ADCs.

To further understand the resistance of endothelial cells to anti-TF ADCs, cell cycle progression was evaluated 24 h after addition of the cytokines and TF-specific ADCs. Arrest at the G₂/M phase of the cell cycle was analyzed as previously described in Theunissen et al, Methods Enzymol, 2006, 409:251-284. Briefly, low-passage HUVECs (Lifeline Cell Technologies, Frederick, MD, USA), propagated in VascuLife VEGF-Mv Endothelial media (Lifeline Cell Technologies), and HCT-116 cells were seeded on 12-well plates. The next day, media was removed and replaced with fresh media (no cytokines) or media containing 5 ng/man anan anL IL1-beta, 25 ng/mL TNF-alpha and 50 ng/mL VEGF (with cytokines). A titration of MMAE-linked ADCs or free MMAE was added to the cells. After 24 h of treatment, cells were fixed in ice-cold 70% ethanol. Subsequently, the cells were washed with flow cytometry buffer (PBS, 1% FBS, 0.1% Triton) and stained for 1 h with a 1:100 dilution of phospho-Histone H3 (Ser10) (D2C8 PE Conjugate, Cell Signaling Technology). After 2 washes, the cells were treated for 20 min with 100 μg/mL PureLink RNAse A (ThermoFisher Scientific), followed by the addition of the viability dye TO-PRO-3 Iodide (ThermoFisher Scientific). 40,000 events were collected on a Novocyte flow cytometer. In the Flowjo data analysis software cell doublets and aneuploid cells were excluded. The pH3 signal was plotted against DNA content to determine the percentage of pH3-positive cells.

The percentage of pH3-positive cells (% pH3) with titrations of anti-TF ADCs on HUVECs in the absence or presence of inflammatory cytokines is shown in international PCT application PCT/US2019/012427 and U.S. utility application Ser. No. 16/959,652, incorporated herein by reference in their entirety. The percentage of pH3-positive cells (% pH3) with titrations of anti-TF ADCs on HCT-116 cells is shown in international PCT application PCT/US2019/012427 and U.S. utility application Ser. No. 16/959,652, incorporated herein by reference in their entirety.

While the TF-specific ADCs induced an arrest at the G₂/M phase of the cell cycle in HCT-116 cells, the ADCs did not impact cell cycle progression in HUVECs with or without inflammatory cytokine treatment. As in international PCT application PCT/US2019/012427 and U.S. utility application Ser. No. 16/959,652, incorporated herein by reference in their entirety, the percentage of pH3-positive HCT-116 cells increased 5 times after treatment of 25A-vc-MMAE as compared to treatment of Isotype-vc-MMAE.

As shown in international PCT application PCT/US2019/012427 and U.S. utility application Ser. No. 16/959,652, incorporated herein by reference in their entirety, unconjugated MMAE increase the phosphorylation of histone H3 to a similar extent in both HCT-116 cells and HUVECs, indicating that the resistance in endothelial cells is specific for the MMAE-based ADC.

Taken together, these results indicate that the anti-TF ADCs do not affect the viability of HUVECs in the absence or presence of inflammatory cytokines.

### Example 34: Erk Phosphorylation Assay

For assessment of Erk phosphorylation, A431 cells were plated in 6-well plates (Corning) in media overnight. The following day, cells were washed once and serum starved in serum-free media. After starvation, cells were preincubated with 100 nM of anti-TF antibodies for 30 min at 37° C. FVIIa was spiked into the wells at 50 nM and incubated for 20 min at 37° C. for p-ERK induction. After induction, cells were lysed with RIPA Lysis and Extraction Buffer with Halt™ Protease and Phosphatase Inhibitor Cocktail (ThermoFisher Scientific). Western blot was performed with 20 μg of cell lysate using Phospho-p44/42 MAPK (Erkl/2) (Thr202/Tyr204) and p44/42 MAPK (Erkl/2) (137F5) (Cell Signaling Technology) as primary antibodies and Peroxidase AffiniPure Donkey Anti-Rabbit IgG (H+L) (Jackson ImmunoResearch) as a secondary antibody. Non-saturating band intensities for pErk and Erk were measured on an Amersham AI600 (GE Healthcare). Each pErk intensity was normalized against its respective Erk intensity and the no-antibody no-FVIIa sample intensity.

The Western blot results of pErk and Erk are shown in international PCT application PCT/US2019/012427 and U.S. utility application Ser. No. 16/959,652, incorporated herein by reference in their entirety. Treatment with FVIIa induced Erk phosphorylation by 5.2 fold in cell cultures without pretreatment of anti-TF antibodies. The inducation of Erk phosphorylation was ablated by pretreatment with 1F, 39A and 54E (fold induction between 0.8 and 1.2) and attenuated by 29E and the members of groups 25 and 43 (fold induction between 2.0 and 3.4).

This data indicates that anti-TF antibodies inhibit FVIIa-dependent TF signaling when assessing Erk phosphorylation.

### Example 35: Antibody-Dependent Cellular Cytotoxicity (ADCC) Assay

To evaluate ADCC activity, an ADCC Reporter Bioassay Core Kit (Promega) was used following the manufacturer's protocol. Briefly, A431 cells were plated on a microtiter plate (Corning). The following day, the cells were incubated with a ten-point 1:3 dilution titration of anti-TF antibodies or the ADCs starting at 50 nM. An ADCC effector-to-target cell ratio of 8:1 was added to each well and incubated for 6 h at 37° C. Bio-Glo™ Luciferase Assay Reagent was added to each well to measure luminescence on an Envision plate reader (PerkinElmer, Waltham, MA, USA). The mean and standard deviation of 4 replicates were graphed in Prism. For each antibody and ADC, the EC₅₀ and its associated 95% confidence interval were calculated in Prism using a 4-parameter binding model.

ADCC reporter luminescence after incubation with the reporter Jurkat cell line in the represece titrations of anti-TF antibodies or anti-TF ADCs is shown in international PCT application PCT/US2019/012427 and U.S. utility application Ser. No. 16/959,652, incorporated herein by reference in their entirety. The ADCC reporter luminescence EC₅₀ values for each anti-TF antibody or ADC are shown in international PCT application PCT/US2019/012427 and U.S. utility application Ser. No. 16/959,652, incorporated herein by reference in their entirety.

All the tested TF-specific antibodies and ADCs exerted induction of luciferase-dependent luminescence with EC₅₀ values ranging between 0.18 nM and 0.43 nM.

These data indicate that both the TF-specific antibodies and ADCs can induce antibody-dependent cellular cytotoxicity (ADCC) via the IgG1 Fc domain of the antibody.

### Example 36: Binding Affinity Assay for Pig TF and Rabbit TF

The ability of certain antibodies was tested for binding to pig TF. For pig TF Biacore-based measurements, a given anti-TF antibody was captured by an anti-human IgG antibody covalently coupled to a CM5 chip (GE Healthcare Bio-Sciences). Association between the anti-TF antibodies and a five-point three-fold titration of pig TF-His starting at 100 nM was measured for 180 to 240 sec. Subsequently, dissociation between the anti-TF antibody and TF-His was measured for 1800 sec. Kinetic data was analyzed and fitted globally using a 1:1 binding model. The K_{D} values of the indicated TF antibodies measured by the Biacore-based experiments are shown in Table 48.

The ability of certain antibodies was tested for binding to rabbit TF. For rabbit TF Biacore-based measurements, a given anti-TF antibody was captured by an anti-human IgG antibody covalently coupled to a CM5 chip (GE Healthcare Bio-Sciences). Association between the anti-TF antibodies and a five-point three-fold titration of rabbit TF-His starting at 100 nM was measured for 180 to 240 sec. Subsequently, dissociation between the anti-TF antibody and TF-His was measured for 1800 sec. Kinetic data was analyzed and fitted globally using a 1:1 binding model. The K_{D} values of the indicated TF antibodies measured by the Biacore-based experiments are shown in Table 48.

As shown in Table 48, anti-hTF antibodies from groups 25 and 43 exhibit binding activity and cross-reactivity to pig TF and rabbit TF. In contrast, antibodies from groups 1 and 29 show no binding activity to pig TF or rabbit TF.

TABLE 48
Antibody kinetics for pig and rabbit TF

| Antibody | Pig K_{D}, nM | Rabbit K_{D}, nM |
|---|---|---|
| 1G | no binding | no binding |
| 25A | 18.7 | 50.5 |
| 25A3 | 5.5 | 12.4 |
| 25A5 | 5.2 | 5.4 |
| 25A5-T | 4.5 | 5.4 |
| 25G | 26.0 | 75.5 |
| 25G1 | 2.6 | 3.6 |
| 25G9 | 3.3 | 4.2 |
| 29D | no binding | no binding |
| 43D7 | 8.8 | 6.8 |
| 43D8 | 19.2 | 7.7 |

no binding*: no binding to weak binding, with no reportable K_{D}

### Example 37: Epitope Binning of Anti-TF Antibodies

To establish epitope binding differences between the anti-human TF antibodies, chimeric TF construct mapping experiments were conducted. This mapping technique enables discrimination of antibody epitopes.

Because all the anti-human TF antibodies evaluated do not bind rat TF, the rat TF sequence was used for the construction of chimeric human-rat TF constructs. Chimeric human-rat construct design was guided by the N- and C-terminal domain of TF extracellular domain (amino acids 1-107 and 108-219 of the extracellular domain, respectively), with an alignment shown in international PCT application PCT/US2019/012427 and U.S. utility application Ser. No. 16/959,652, incorporated herein by reference in their entirety. Based on the chimera mapping results using the constructs from FIG. 36 of international PCT application PCT/US2019/012427 and U.S. utility application Ser. No. 16/959,652, rat amino acid segment 141-194 was replaced by the human sequence (amino acid 136-189 of hTF extracellular domain), with an alignment shown in international PCT application PCT/US2019/012427 and U.S. utility application Ser. No. 16/959,652, incorporated herein by reference in their entirety. Design of three human TF constructs with either 1 or 2 human-rat substitutions (hTF_K68N, hTF_K149N and hTF_N171H_T197K) was based on reported contact residues K68, K149 and N171 and T197 for the 10H10 antibody (Teplyakov et al., Cell Signal., 2017, 36:139-144), with an alignment shown in international PCT application PCT/US2019/012427 and U.S. utility application Ser. No. 16/959,652, incorporated herein by reference in their entirety.

To establish binding of the anti-human TF antibodies to the various TF constructs, HEK293 cells were transfected with a DNA plasmid that co-expresses the TF construct and a green fluorescent protein marker. For a subset of the antibodies, an antibody titration (a 12-point 1:3 dilution series starting at 250 nM) was evaluated on select TF constructs (See international PCT application PCT/US2019/012427 and U.S. utility application Ser. No. 16/959,652, incorporated herein by reference in their entirety). These antibody titrations demonstrated that the antibody concentration of 15 μg/ml (100 nM) used in Tables 51 and 52 was appropriate to establish “Percentage antibody binding to TF construct relative to hTF”. Two days after transfection, cells were collected from the tissue culture plate, stained with 15 μg/ml of the indicated anti-TF antibody, washed, stained with anti-human IgG-Fc Alexa Fluor 647 polyclonal antibody, washed, and stained with the viability dye 4′,6-Diamidino-2-Phenylindole, Dihydrochloride. Upon acquisition of 80,000 live events on a flow cytometer, live cells marked with the fluorescent marker were analyzed for the degree of staining by the anti-TF antibody. The median fluorescence intensity values relative to an isotype control for each TF expression construct were divided by the median fluorescence intensity value relative to an isotype control for the hTF expression construct, and the resulting percentage listed as “Percentage antibody binding to TF construct relative to hTF” in Tables 51 and 52. As used herein, the term “live cell staining assay” refers to the antibody binding assay used in this example.

The assumption that all chimeric TF constructs were expressed on the cell surface at levels between 50% and 150% of the hTF control construct was met for all TF constructs for at least one anti-human TF antibody in the antibody collection, with the exception of the h1-107_r construct (human amino acid segment 1-107 replaced by rat sequence). Lack of binding of the anti-human TF antibodies to cell surface-expressed rat TF was expected. When “Percentage antibody binding to TF construct relative to hTF” in Tables 51 and 52 was less than 50%, the antibody was considered a non-binder (0) in Tables 53 and 54. When “Percentage antibody binding to TF construct relative to hTF” in Tables 51 and 52 was between 50% and 150%, the antibody was considered a binder (1) in Tables 53 and 54.

Each antibody was assigned to an epitope bin in Table 55 based on the combination of unbound constructs from Table 53. The antibodies from Lineage 25 (25A, 25A3, 25A5-T, 25G1 and 25G9) bind a unique epitope, referred to as Epitope Bin 6 in Table 55. The antibodies from Lineage 43 (43B1, 43D7, 43D8 and 43Ea) also bind a unique epitope, referred to as Epitope Bin 7 in Table 55. The antibody from Lineage 29 (29E) binds a unique epitope, referred to as Epitope Bin 2 in Table 55. The antibodies from Lineage 39 and 54 (39A and 54E) bind a unique epitope, referred to as Epitope Bin 3 in Table 55.

Lineage 25 and 43 antibodies are the only antibodies in the antibody panel that bind r141-194_h, the chimeric construct in which rat amino acids 141-194 were replaced by human sequence (Table 54). Furthermore, while M1593 cannot bind hTF_K68N, all the other antibodies in the antibody panel bind hTF_K68N (Table 54). Only Lineage 25 and 43 antibodies cannot bind hTF_K149N (Table 54). Only Lineage 25 antibodies cannot bind hTF_N171H_T197K (Table 54). (See international PCT application PCT/US2019/012427 and U.S. utility application Ser. No. 16/959,652, incorporated herein by reference in their entirety).

In summary, these results indicate that lineage 25 antibodies bind a unique epitope on human TF compared to all other antibodies tested. Lineage 43 antibodies bind a unique epitope on human TF compared to all other antibodies tested. Lineage 25 and lineage 43 antibodies bind a different epitope on human TF from M1593.

TABLE 51
Percent antibody binding to TF construct relative to hTF

| | | Antibody | | | | | | | |
|---|---|---|---|---|---|---|---|---|---|
| | Construct | 1F | 29E | 39A | 54E | TF-011 | 5G9 | M1593 | 25A |
| Human | hTF | 100 | 100 | 100 | 100 | 100 | 100 | 100 | 100 |
| amino | rTF | 0 | 0 | 0 | 0 | 0 | 0 | 0 | 0 |
| acid | h1-107_r (52) | 0 | 0 | 0 | 0 | 0 | 41 | 0 | 32 |
| segment | h1-77_r (25) | 0 | 0 | 0 | 0 | 0 | 94 | 0 | 86 |
| replaced | h1-38_r (14) | 91 | 87 | 100 | 102 | 104 | 100 | 104 | 101 |
| by rat | h39-77_r (11) | 0 | 0 | 0 | 0 | 0 | 88 | 2 | 82 |
| segment (in | h78-107_r (21) | 0 | 8 | 81 | 68 | 32 | 114 | 74 | 108 |
| parentheses: | h78-107_r.v2 (27) | 0 | 0 | 76 | 62 | 23 | 101 | 59 | 95 |
| number of | h78-93_r (18) | 102 | 0 | 77 | 91 | 110 | 102 | 104 | 106 |
| amino acid | h94-107_r (9) | 1 | 82 | 85 | 89 | 27 | 91 | 46 | 82 |
| changes | h108-219_r (46) | 119 | 118 | 118 | 122 | 128 | 0 | 0 | 0 |
| relative to | h108-158_r (19) | 98 | 101 | 107 | 108 | 108 | 63 | 4 | 1 |
| human TF) | h108-132_r (10) | 105 | 108 | 109 | 107 | 124 | 125 | 124 | 112 |
| | h133-158_r (9) | 113 | 122 | 119 | 130 | 134 | 91 | 0 | 0 |
| | h133-145_r (4) | 84 | 95 | 96 | 104 | 104 | 108 | 100 | 77 |
| | h133-139_r (2) | 82 | 90 | 95 | 103 | 102 | 104 | 103 | 88 |
| | h140-145_r (2) | 89 | 100 | 101 | 110 | 109 | 113 | 97 | 80 |
| | h146-158_r (5) | 115 | 122 | 125 | 134 | 134 | 91 | 133 | 2 |
| | h146-151_r (1) | 122 | 133 | 139 | 142 | 143 | 141 | 118 | 3 |
| | h152-158_r (4) | 110 | 121 | 128 | 127 | 136 | 82 | 132 | 110 |
| | h159-219_r (27) | 132 | 134 | 141 | 142 | 155 | 0 | 137 | 0 |
| | h159-189_r (11) | 94 | 101 | 104 | 110 | 112 | 0 | 105 | 0 |
| | h159-174_r (6) | 96 | 98 | 101 | 118 | 120 | 0 | 98 | 0 |
| | h159-166_r (3) | 89 | 93 | 96 | 100 | 98 | 104 | 100 | 93 |
| | h167-174_r (3) | 96 | 112 | 96 | 122 | 128 | 0 | 118 | 0 |
| | h175-189_r (5) | 97 | 113 | 112 | 118 | 123 | 119 | 114 | 86 |
| | h190-219_r (16) | 111 | 138 | 149 | 141 | 145 | 12 | 143 | 125 |

| | | Antibody | | | | | | | |
|---|---|---|---|---|---|---|---|---|---|
| | Construct | 25A3 | 25A5-T | 25G1 | 25G9 | 43B1 | 43D7 | 43D8 | 43Ea |
| Human | hTF | 100 | 100 | 100 | 100 | 100 | 100 | 100 | 100 |
| amino | rTF | 0 | 0 | 0 | 0 | 0 | 0 | 0 | 0 |
| acid | h1-107_r (52) | 36 | 36 | 37 | 28 | 33 | 35 | 31 | 37 |
| segment | h1-77_r (25) | 95 | 84 | 88 | 64 | 64 | 75 | 69 | 69 |
| replaced | h1-38_r (14) | 104 | 93 | 101 | 88 | 97 | 106 | 104 | 103 |
| by rat | h39-77_r (11) | 88 | 80 | 87 | 71 | 59 | 75 | 71 | 69 |
| segment (in | h78-107_r (21) | 116 | 103 | 113 | 108 | 113 | 114 | 117 | 114 |
| parentheses: | h78-107_r.v2 (27) | 96 | 91 | 94 | 93 | 97 | 100 | 101 | 101 |
| number of | h78-93_r (18) | 105 | 92 | 101 | 98 | 101 | 104 | 102 | 103 |
| amino acid | h94-107_r (9) | 86 | 78 | 83 | 77 | 84 | 92 | 89 | 91 |
| changes | h108-219_r (46) | 0 | 0 | 0 | 0 | 0 | 0 | 0 | 0 |
| relative to | h108-158_r (19) | 0 | 0 | 11 | 22 | 0 | 1 | 0 | 0 |
| human TF) | h108-132_r (10) | 112 | 106 | 111 | 118 | 122 | 126 | 122 | 124 |
| | h133-158_r (9) | 0 | 0 | 2 | 3 | 0 | 4 | 1 | 0 |
| | h133-145_r (4) | 80 | 80 | 87 | 100 | 99 | 104 | 103 | 106 |
| | h133-139_r (2) | 89 | 88 | 91 | 86 | 94 | 101 | 97 | 101 |
| | h140-145_r (2) | 87 | 86 | 89 | 105 | 101 | 104 | 104 | 109 |
| | h146-158_r (5) | 17 | 18 | 17 | 0 | 3 | 20 | 10 | 0 |
| | h146-151_r (1) | 14 | 17 | 7 | 0 | 11 | 39 | 23 | 2 |
| | h152-158_r (4) | 116 | 112 | 116 | 111 | 119 | 134 | 129 | 134 |
| | h159-219_r (27) | 0 | 0 | 0 | 0 | 132 | 130 | 130 | 76 |
| | h159-189_r (11) | 0 | 0 | 0 | 0 | 100 | 106 | 104 | 94 |
| | h159-174_r (6) | 0 | 0 | 0 | 0 | 103 | 115 | 112 | 101 |
| | h159-166_r (3) | 95 | 87 | 91 | 88 | 99 | 106 | 105 | 110 |
| | h167-174_r (3) | 0 | 0 | 0 | 0 | 109 | 121 | 112 | 104 |
| | h175-189_r (5) | 95 | 99 | 100 | 86 | 109 | 118 | 114 | 122 |
| | h190-219_r (16) | 124 | 119 | 127 | 144 | 133 | 140 | 136 | 147 |

TABLE 52
Percent antibody binding to TF construct relative to hTF

| | Antibody | | | | | | | | | | | | | | | |
|---|---|---|---|---|---|---|---|---|---|---|---|---|---|---|---|---|
| Construct | 1F | 29E | 39A | 54E | TF-011 | 5G9 | M1593 | 25A | 25A3 | 25A5-T | 25G1 | 25G9 | 43B1 | 43D7 | 43D8 | 43Ea |
| hTF | 100 | 100 | 100 | 100 | 100 | 100 | 100 | 100 | 100 | 100 | 100 | 100 | 100 | 100 | 100 | 100 |
| rTF | 0 | 0 | 0 | 0 | 0 | 0 | 0 | 0 | 0 | 0 | 0 | 0 | 0 | 0 | 0 | 0 |
| r141-194_h* | 0 | 0 | 0 | 0 | 0 | 32 | 0 | 65 | 89 | 88 | 83 | 108 | 90 | 102 | 95 | 81 |
| hTF_K68N | 105 | 115 | 119 | 118 | 111 | 132 | 0 | 93 | 124 | 126 | 115 | 103 | 107 | 116 | 119 | 118 |
| hTF_K149N | 115 | 117 | 131 | 127 | 132 | 145 | 111 | 2 | 12 | 13 | 7 | 0 | 10 | 29 | 20 | 1 |
| hTF_N171H_T197K | 83 | 98 | 94 | 89 | 109 | 102 | 113 | 1 | 4 | 7 | 1 | 0 | 98 | 101 | 103 | 118 |

*rat amino acid segment replaced by human segment, resulting in 20 amino acid changes

TABLE 53
Antibody binding to TF construct

| | | Antibody | | | | | | | |
|---|---|---|---|---|---|---|---|---|---|
| | Construct | 1F | 29E | 39A | 54E | TF-011 | 5G9 | M1593 | 25A |
| Human amino | hTF | 1 | 1 | 1 | 1 | 1 | 1 | 1 | 1 |
| acid segment | rTF | 0 | 0 | 0 | 0 | 0 | 0 | 0 | 0 |
| replaced by | h1-107_r (52) | 0 | 0 | 0 | 0 | 0 | 0 | 0 | 0 |
| rat segment | h1-77_r (25) | 0 | 0 | 0 | 0 | 0 | 1 | 0 | 1 |
| (in parentheses: | h1-38_r (14) | 1 | 1 | 1 | 1 | 1 | 1 | 1 | 1 |
| number of amino | h39-77_r (11) | 0 | 0 | 0 | 0 | 0 | 1 | 0 | 1 |
| acid changes | h78-107_r (21) | 0 | 0 | 1 | 1 | 0 | 1 | 1 | 1 |
| relative to | h78-107_r.v2 (27) | 0 | 0 | 1 | 1 | 0 | 1 | 1 | 1 |
| human TF) | h78-93_r (18) | 1 | 0 | 1 | 1 | 1 | 1 | 1 | 1 |
| | h94-107_r (9) | 0 | 1 | 1 | 1 | 0 | 1 | 0 | 1 |
| | h108-219_r (46) | 1 | 1 | 1 | 1 | 1 | 0 | 0 | 0 |
| | h108-158_r (19) | 1 | 1 | 1 | 1 | 1 | 1 | 0 | 0 |
| | h108-132_r (10) | 1 | 1 | 1 | 1 | 1 | 1 | 1 | 1 |
| | h133-158_r (9) | 1 | 1 | 1 | 1 | 1 | 1 | 0 | 0 |
| | h133-145_r (4) | 1 | 1 | 1 | 1 | 1 | 1 | 1 | 1 |
| | h133-139_r (2) | 1 | 1 | 1 | 1 | 1 | 1 | 1 | 1 |
| | h140-145_r (2) | 1 | 1 | 1 | 1 | 1 | 1 | 1 | 1 |
| | h146-158_r (5) | 1 | 1 | 1 | 1 | 1 | 1 | 1 | 0 |
| | h146-151_r (1) | 1 | 1 | 1 | 1 | 1 | 1 | 1 | 0 |
| | h152-158_r (4) | 1 | 1 | 1 | 1 | 1 | 1 | 1 | 1 |
| | h159-219_r (27) | 1 | 1 | 1 | 1 | 1 | 0 | 1 | 0 |
| | h159-189_r (11) | 1 | 1 | 1 | 1 | 1 | 0 | 1 | 0 |
| | h159-174_r (6) | 1 | 1 | 1 | 1 | 1 | 0 | 1 | 0 |
| | h159-166_r (3) | 1 | 1 | 1 | 1 | 1 | 1 | 1 | 1 |
| | h167-174_r (3) | 1 | 1 | 1 | 1 | 1 | 0 | 1 | 0 |
| | h175-189_r (5) | 1 | 1 | 1 | 1 | 1 | 1 | 1 | 1 |
| | h190-219_r (16) | 1 | 1 | 1 | 1 | 1 | 0 | 1 | 1 |

| | | Antibody | | | | | | | |
|---|---|---|---|---|---|---|---|---|---|
| | Construct | 25A3 | 25A5-T | 25G1 | 25G9 | 43B1 | 43D7 | 43D8 | 43Ea |
| Human amino | hTF | 1 | 1 | 1 | 1 | 1 | 1 | 1 | 1 |
| acid segment | rTF | 0 | 0 | 0 | 0 | 0 | 0 | 0 | 0 |
| replaced by | h1-107_r (52) | 0 | 0 | 0 | 0 | 0 | 0 | 0 | 0 |
| rat segment | h1-77_r (25) | 1 | 1 | 1 | 1 | 1 | 1 | 1 | 1 |
| (in parentheses: | h1-38_r (14) | 1 | 1 | 1 | 1 | 1 | 1 | 1 | 1 |
| number of amino | h39-77_r (11) | 1 | 1 | 1 | 1 | 1 | 1 | 1 | 1 |
| acid changes | h78-107_r (21) | 1 | 1 | 1 | 1 | 1 | 1 | 1 | 1 |
| relative to | h78-107_r.v2 (27) | 1 | 1 | 1 | 1 | 1 | 1 | 1 | 1 |
| human TF) | h78-93_r (18) | 1 | 1 | 1 | 1 | 1 | 1 | 1 | 1 |
| | h94-107_r (9) | 1 | 1 | 1 | 1 | 1 | 1 | 1 | 1 |
| | h108-219_r (46) | 0 | 0 | 0 | 0 | 0 | 0 | 0 | 0 |
| | h108-158_r (19) | 0 | 0 | 0 | 0 | 0 | 0 | 0 | 0 |
| | h108-132_r (10) | 1 | 1 | 1 | 1 | 1 | 1 | 1 | 1 |
| | h133-158_r (9) | 0 | 0 | 0 | 0 | 0 | 0 | 0 | 0 |
| | h133-145_r (4) | 1 | 1 | 1 | 1 | 1 | 1 | 1 | 1 |
| | h133-139_r (2) | 1 | 1 | 1 | 1 | 1 | 1 | 1 | 1 |
| | h140-145_r (2) | 1 | 1 | 1 | 1 | 1 | 1 | 1 | 1 |
| | h146-158_r (5) | 0 | 0 | 0 | 0 | 0 | 0 | 0 | 0 |
| | h146-151_r (1) | 0 | 0 | 0 | 0 | 0 | 0 | 0 | 0 |
| | h152-158_r (4) | 1 | 1 | 1 | 1 | 1 | 1 | 1 | 1 |
| | h159-219_r (27) | 0 | 0 | 0 | 0 | 1 | 1 | 1 | 1 |
| | h159-189_r (11) | 0 | 0 | 0 | 0 | 1 | 1 | 1 | 1 |
| | h159-174_r (6) | 0 | 0 | 0 | 0 | 1 | 1 | 1 | 1 |
| | h159-166_r (3) | 1 | 1 | 1 | 1 | 1 | 1 | 1 | 1 |
| | h167-174_r (3) | 0 | 0 | 0 | 0 | 1 | 1 | 1 | 1 |
| | h175-189_r (5) | 1 | 1 | 1 | 1 | 1 | 1 | 1 | 1 |
| | h190-219_r (16) | 1 | 1 | 1 | 1 | 1 | 1 | 1 | 1 |

TABLE 54
Antibody binding to TF construct

| | Antibody | | | | | | | | | | | | | | | |
|---|---|---|---|---|---|---|---|---|---|---|---|---|---|---|---|---|
| Construct | 1F | 29E | 39A | 54E | TF-011 | 5G9 | M1593 | 25A | 25A3 | 25A5-T | 25G1 | 25G9 | 43B1 | 43D7 | 43D8 | 43Ea |
| hTF | 1 | 1 | 1 | 1 | 1 | 1 | 1 | 1 | 1 | 1 | 1 | 1 | 1 | 1 | 1 | 1 |
| rTF | 0 | 0 | 0 | 0 | 0 | 0 | 0 | 0 | 0 | 0 | 0 | 0 | 0 | 0 | 0 | 0 |
| r141-194_h* | 0 | 0 | 0 | 0 | 0 | 0 | 0 | 1 | 1 | 1 | 1 | 1 | 1 | 1 | 1 | 1 |
| hTF_K68N | 1 | 1 | 1 | 1 | 1 | 1 | 0 | 1 | 1 | 1 | 1 | 1 | 1 | 1 | 1 | 1 |
| hTF_K149N | 1 | 1 | 1 | 1 | 1 | 1 | 1 | 0 | 0 | 0 | 0 | 0 | 0 | 0 | 0 | 0 |
| hTF_N171H_T197K | 1 | 1 | 1 | 1 | 1 | 1 | 1 | 0 | 0 | 0 | 0 | 0 | 1 | 1 | 1 | 1 |

*rat amino acid segment replaced by human segment, resulting in 20 amino acid changes

TABLE 55
Epitope Bin assignment based on unbound chimeric constructs

| | | Epitope |
|---|---|---|
| Antibody | Constructs not bound by antibody | Bin |
| 1F | rTF, h1-107_r, h1-77_r, h39-77_r, h78-107_r, h78-107_r.v2, h94-107_r | 1 |
| 29E | rTF, h1-107_r, h1-77_r, h39-77_r, h78-107_r, h78-107_r.v2, h78-93_r | 2 |
| 39A | rTF, h1-107_r, h1-77_r, h39-77_r | 3 |
| 54E | rTF, h1-107_r, h1-77_r, h39-77_r | 3 |
| TF-011 | rTF, h1-107_r, h1-77_r, h39-77_r, h78-107_r, h78-107_r.v2, h94-107_r | 1 |
| 5G9 | rTF, h1-107_r, h108-219_r, h159-219_r, h159-189_r, h159-174_r, h167-174_r, h190-219_r | 4 |
| M1593 | rTF, h1-107_r, h1-77_r, h39-77_r, h94-107_r, h108-219_r, h108-158_r, h133-158_r | 5 |
| 25A | rTF, h1-107_r, h108-219_r, h108-158_r, h133-158_r, h146-158_r, h146-151_r, h159-219_r, | 6 |
| | h159-189_r, h159-174_r, h167-174_r | |
| 25A3 | rTF, h1-107_r, h108-219_r, h108-158_r, h133-158_r, h146-158_r, h146-151_r, h159-219_r, | 6 |
| | h159-189_r, h159-174_r, h167-174_r | |
| 25A5-T | rTF, h1-107_r, h108-219_r, h108-158_r, h133-158_r, h146-158_r, h146-151_r, h159-219_r, | 6 |
| | h159-189_r, h159-174_r, h167-174_r | |
| 25G1 | rTF, h1-107_r, h108-219_r, h108-158_r, h133-158_r, h146-158_r, h146-151_r, h159-219_r, | 6 |
| | h159-189_r, h159-174_r, h167-174_r | |
| 25G9 | rTF, h1-107_r, h108-219_r, h108-158_r, h133-158_r, h146-158_r, h146-151_r, h159-219_r, | 6 |
| | h159-189_r, h159-174_r, h167-174_r | |
| 43B1 | rTF, h1-107_r, h108-219_r, h108-158_r, h133-158_r, h146-158_r, h146-151_r | 7 |
| 43D7 | rTF, h1-107_r, h108-219_r, h108-158_r, h133-158_r, h146-158_r, h146-151_r | 7 |
| 43D8 | rTF, h1-107_r, h108-219_r, h108-158_r, h133-158_r, h146-158_r, h146-151_r | 7 |
| 43Ea | rTF, h1-107_r, h108-219_r, h108-158_r, h133-158_r, h146-158_r, h146-151_r | 7 |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

TABLE 13
SEQUENCES
Variable region sequences

| Clone | VH Domains (SEQ ID NO) | VL Domains (SEQ ID NO) |
|---|---|---|
| 1F | EVQLLESGGGLVQPGGSLRLSCAASGF | DIQMTQSPSTLSASVGDRVTITCRASQS |
| | TFSDYAMGWVRQAPGKGLEWVSTISG | ISSWLAWYQQKPGKAPKLLIYKASSLE |
| | SGGLTYYADSVKGRFTISRDNSKNTLY | SGVPSRFSGSGSGTEFTLTISSLQPDDF |
| | LQMNSLRAEDTAVYYCAKAPYGYYM | ATYYCQQYKSYITFGGGTKVEIK |
| | DVWGKGTTVTVSS (SEQ ID NO: 37) | (SEQ ID NO: 38) |
| 1G | EVQLLESGGGLVQPGGSLRLSCAASGF | DIQMTQSPSTLSASVGDRVTITCRASQS |
| | TFSSYAMAWVRQAPGKGLEWVSAISG | ISSWLAWYQQKPGKAPKLLIYKASSLE |
| | SGGLTYYADSVKGRFTISRDNSKNTLY | SGVPSRFSGSGSGTEFTLTISSLQPDDF |
| | LQMNSLRAEDTAVYYCAKAPYGYYM | ATYYCQQYKSYITFGGGTKVEIK |
| | DVWGKGTTVTVSS (SEQ ID NO: 75) | (SEQ ID NO: 76) |
| 25A | QVQLVQSGAEVKKPGASVKVSCKASG | DIQMTQSPSTLSASVGDRVTITCRASQS |
| | YTFDVYGISWVRQAPGQGLEWMGWI | ISSWLAWYQQKPGKAPKLLIYKASSLE |
| | APYNGNTNYAQKLQGRVTMTTDTSTS | SGVPSRFSGSGSGTEFTLTISSLQPDDF |
| | TAYMELRSLRSDDTAVYYCARDAGTY | ATYYCQQFQSLPPFTFGGGTKVEIK |
| | SPFGYGMDVWGQGTTVTVSS (SEQ ID | (SEQ ID NO: 114) |
| | NO: 113) | |
| 25A3 | QVQLVQSGAEVKKPGASVKVSCKASG | DIQMTQSPSTLSASVGDRVTITCQASQS |
| | YTFDVYGISWVRQAPGQGLEWMGWI | INNWLAWYQQKPGKAPKLLIYKAYNL |
| | APYSGNTNYAQKLQGRVTMTTDTSTS | ESGVPSRFSGSGSGTEFTLTISSLQPDDF |
| | TAYMELRSLRSDDTAVYYCARDAGTY | ATYYCQLFQSLPPFTFGGGTKVEIK |
| | SPFGYGMDVWGQGTTVTVSS (SEQ ID | (SEQ ID NO: 152) |
| | NO: 151) | |
| 25A5 | QVQLVQSGAEVKKPGASVKVSCKASG | DIQMTQSPSTLSASVGDRVTITCRASES |
| | YTFDVYGISWVRQAPGQGLEWMGWI | ISNWLAWYQQKPGKAPKLLIYKAYSL |
| | APYSGNTNYAQKLQGRVTMTTDTSTS | EYGVPSRFSGSGSGTEFTLTISSLQPDD |
| | TAYMELRSLRSDDTAVYYCARDAGTY | FATYYCQQFQKLPPFTFGGGTKVEIK |
| | SPFGYGMDVWGQGTTVTVSS (SEQ ID | (SEQ ID NO: 190) |
| | NO: 189) | |
| 25A5-T | QVQLVQSGAEVKKPGASVKVSCKASG | DIQMTQSPSTLSASVGDRVTITCRASES |
| | YTFDAYGISWVRQAPGQGLEWMGWI | ISNWLAWYQQKPGKAPKLLIYKAYSL |
| | APYSGNTNYAQKLQGRVTMTTDTSTS | EYGVPSRFSGSGSGTEFTLTISSLQPDD |
| | TAYMELRSLRSDDTAVYYCARDAGTY | FATYYCQQFQKLPPFTFGGGTKVEIK |
| | SPFGYGMDVWGQGTTVTVSS (SEQ ID | (SEQ ID NO: 837) |
| | NO: 836) | |
| 25G | QVQLVQSGAEVKKPGASVKVSCKASG | DIQMTQSPSTLSASVGDRVTITCRASQS |
| | YTFRSYGISWVRQAPGQGLEWMGWV | ISSWLAWYQQKPGKAPKLLIYKASSLE |
| | APYNGNTNYAQKLQGRVTMTTDTSTS | SGVPSRFSGSGSGTEFTLTISSLQPDDF |
| | TAYMELRSLRSDDTAVYYCARDAGTY | ATYYCQQFQSLPPFTFGGGTKVEIK |
| | SPYGYGMDVWGQGTTVTVSS (SEQ ID | (SEQ ID NO: 228) |
| | NO: 227) | |
| 25G1 | QVQLVQSGAEVKKPGASVKVSCKASG | DIQMTQSPSTLSASVGDRVTITCRASHS |
| | YTFRSYGISWVRQAPGQGLEWMGWV | IDSWLAWYQQKPGKAPKLLIYKASYL |
| | APYSGNTNYAQKLQGRVTMTTDTSTS | ESGVPSRFSGSGSGTEFTLTISSLQPDDF |
| | TAYMELRSLRSDDTAVYYCARDAGTY | ATYYCQLFQSLPPFTFGGGTKVEIK |
| | SPYGYGMDVWGQGTTVTVSS (SEQ ID | (SEQ ID NO: 266) |
| | NO: 265) | |
| 25G9 | QVQLVQSGAEVKKPGASVKVSCKASG | DIQMTQSPSTLSASVGDRVTITCQASQS |
| | YTFRSYGISWVRQAPGQGLEWMGWV | IDSWLAWYQQKPGKAPKLLIYSASYLE |
| | APYSGNTNYAQKLQGRVTMTTDTSTS | SGVPSRFSGSGSGTEFTLTISSLQPDDF |
| | TAYMELRSLRSDDTAVYYCARDAGTY | ATYYCQRFQSLPPFTFGGGTKVEIK |
| | SPYGYGMDVWGQGTTVTVSS (SEQ ID | (SEQ ID NO: 304) |
| | NO: 303) | |
| 29D | QVQLVESGGGVVQPGRSLRLSCAASG | DIVMTQSPDSLAVSLGERATINCKSSQS |
| | FTFHSRGMHWVRQAPGKGLEWVAVIT | VLFSSNNKNYLAWYQQKPGQPPKLLI |
| | YDGINKYYADSVEGRFTISRDNSKNTL | YWASTRESGVPDRFSGSGSGTDFTLTIS |
| | YLQMNSLRAEDTAVYYCARDGVYYG | SLQAEDVAVYYCQQFHSYPLTFGGGT |
| | VYDYWGQGTLVTVSS (SEQ ID | KVEIK (SEQ ID NO: 342) |
| | NO: 341) | |
| 29E | QVQLVESGGGVVQPGRSLRLSCAASG | DIVMTQSPDSLAVSLGERATINCKSSQS |
| | FTFRSYGMHWVRQAPGKGLEWVAVIT | VLFSSNNKNYLAWYQQKPGQPPKLLI |
| | YDGINKYYADSVEGRFTISRDNSKNTL | YWASTRESGVPDRFSGSGSGTDFTLTIS |
| | YLQMNSLRAEDTAVYYCARDGVYYG | SLQAEDVAVYYCQQFHSYPLTFGGGT |
| | VYDYWGQGTLVTVSS (SEQ ID | KVEIK (SEQ ID NO: 380) |
| | NO: 379) | |
| 39A | QVQLVQSGAEVKKPGSSVKVSCKASG | EIVMTQSPATLSVSPGERATLSCRASQS |
| | GTFSSNAIGWVRQAPGQGLEWMGSIIP | VSSNLAWYQQKPGQAPRLLIYGASTR |
| | IIGFANYAQKFQGRVTITADESTSTAY | ATGIPARFSGSGSGTEFTLTISSLQSEDF |
| | MELSSLRSEDTAVYYCARDSGYYYGA | AVYYCEQYNNLPLTFGGGTKVEIK |
| | SSFGMDVWGQGTTVTVSS (SEQ ID | (SEQ ID NO: 418) |
| | NO: 417) | |
| 43B | QVQLQESGPGLVKPSQTLSLTCTVSGG | EIVLTQSPGTLSLSPGERATLSCRASQS |
| | SISSGQYWSWIRQHPGKGLEWIGEIYY | VSSSYLAWYQQKPGQAPRLLIYGASSR |
| | SGSTRYNPSLKSRVTISVDTSKNQFSLK | ATGIPDRFSGSGSGTDFTLTISRLEPEDF |
| | LSSVTAADTAVYYCARDAPYYYGGGY | AVYYCQQVGVVPYTFGGGTKVEIK |
| | YYYMDVWGKGTTVTVSS (SEQ ID | (SEQ ID NO: 456) |
| | NO: 455) | |
| 43B1 | QVQLQESGPGLVKPSQTLSLTCTVSGG | EIVLTQSPGTLSLSPGERATLSCRASES |
| | SISSGQYWSWIRQHPGKGLEWIGEIYY | VDSSYLAWYQQKPGQAPRLLIYGAST |
| | SGSTRYNPSLKSRVTISVDTSKNQFSLK | RQTGIPDRFSGSGSGTDFTLTISRLEPED |
| | LSSVTAADTAVYYCARDAPYYYGGGY | FAVYYCQQAGVVPYTFGGGTKVEIK |
| | YYYMDVWGKGTTVTVSS (SEQ ID | (SEQ ID NO: 494) |
| | NO: 493) | |
| 43B7 | QVQLQESGPGLVKPSQTLSLTCTVSGG | EIVLTQSPGTLSLSPGERATLSCRASES |
| | SISSGQYWSWIRQHPGKGLEWIGEIYY | VDSSYLAWYQQKPGQAPRLLIYGADS |
| | SGSTRYNPSLKSRVTISVDTSKNQFSLK | RATGIPDRFSGSGSGTDFTLTISRLEPED |
| | LSSVTAADTAVYYCARDAPYYYGGGY | FAVYYCQQDGVVPYTFGGGTKVEIK |
| | YYYMDVWGKGTTVTVSS (SEQ ID | (SEQ ID NO: 532) |
| | NO: 531) | |
| 43D | QVQLQQWGAGLLKPSETLSLTCAVYG | EIVLTQSPGTLSLSPGERATLSCRASQS |
| | GSLSGYYWSWIRQPPGKGLEWIGEIGA | VSSSYLAWYQQKPGQAPRLLIYGASSR |
| | SGSTRYNPSLKSRVTISVDTSKNQFSLK | ATGIPDRFSGSGSGTDFTLTISRLEPEDF |
| | LSSVTAADTAVYYCARDTPYYYEGGY | AVYYCQQVGVVPYTFGGGTKVEIK |
| | YYYMDVWGKGTTVTVSS (SEQ ID | (SEQ ID NO: 570) |
| | NO: 569) | |
| 43D7 | QVQLQQWGAGLLKPSETLSLTCAVYG | EIVLTQSPGTLSLSPGERATLSCRASDS |
| | GSLSGYYWSWIRQPPGKGLEWIGEIGA | VDSSYLAWYQQKPGQAPRLLIYGAFS |
| | SGSTRYNPSLKSRVTISVDTSKNQFSLK | RANGIPDRFSGSGSGTDFTLTISRLEPE |
| | LSSVTAADTAVYYCARDTPYYYEGGY | DFAVYYCQQAGVVPYTFGGGTKVEIK |
| | YYYMDVWGKGTTVTVSS (SEQ ID | (SEQ ID NO: 608) |
| | NO: 607) | |
| 43D8 | QVQLQQWGAGLLKPSETLSLTCAVYG | EIVLTQSPGTLSLSPGERATLSCRASQS |
| | GSLSGYYWSWIRQPPGKGLEWIGEIGA | VSSSFLAWYQQKPGQAPRLLIYGAYSR |
| | SGSTRYNPSLKSRVTISVDTSKNQFSLK | ATGIPDRFSGSGSGTDFTLTISRLEPEDF |
| | LSSVTAADTAVYYCARDTPYYYEGGY | AVYYCQQAGVVPYTFGGGTKVEIK |
| | YYYMDVWGKGTTVTVSS (SEQ ID | (SEQ ID NO: 646) |
| | NO: 645) | |
| 43E | QVQLQESGPGLVKPSQTLSLTCTVSGG | EIVLTQSPGTLSLSPGERATLSCRASQS |
| | SISSGQYWSWIRQHPGKGLEWIGEIYY | VSSSYLAWYQQKPGQAPRLLIYGASSR |
| | SGSTRYNPSLKSRVTISVDTSKDQFSLK | ATGIPDRFSGSGSGTDFTLTISRLEPEDF |
| | LSSVTAADTAVYYCARDTPYYYDGGY | AVYYCQQVGVVPYTFGGGTKVEIK |
| | YYYMDVWGKGTTVTVSS (SEQ ID | (SEQ ID NO: 684) |
| | NO: 683) | |
| 43Ea | QVQLQESGPGLVKPSQTLSLTCTVSGG | EIVLTQSPGTLSLSPGERATLSCRASQS |
| | SISSGQYWSWIRQHPGKGLEWIGEIYY | VSSSYLAWYQQKPGQAPRLLIYGASSR |
| | SGSTRYNPSLKSRVTISVDTSKNQFSLK | ATGIPDRFSGSGSGTDFTLTISRLEPEDF |
| | LSSVTAADTAVYYCARDTPYYYDGGY | AVYYCQQVGVVPYTFGGGTKVEIK |
| | YYYMDVWGKGTTVTVSS (SEQ ID | (SEQ ID NO: 722) |
| | NO: 721) | |
| 54E | QVQLVQSGAEVKKPGASVKVSCKASG | DIQMTQSPSSLSASVGDRVTITCQASQ |
| | YTFANYYMHWVRQAPGQGLEWMGII | DISNSLNWYQQKPGKAPKLLIYDASNL |
| | NPSGGITVYAQKFQGRVTMTRDTSTST | ETGVPSRFSGSRSGTDFTFTISSLQPEDI |
| | VYMELSSLRSEDTAVYYCARGGSKVA | ATYYCQQYNFHPLTFGGGTKVEIK |
| | ALAFDIWGQGTMVTVSS (SEQ ID | (SEQ ID NO: 760) |
| | NO: 759) | |

TABLE 14
Variable region sequence consensus

| Group | VH Domain Consensus (SEQ ID NO) | VL Domain Consensus (SEQ ID NO) |
|---|---|---|
| 1 | EVQLLESGGGLVQPGGSLRLSCAASGF | DIQMTQSPSTLSASVGDRVTITCRASQ |
| | TFSx[D/S]YAMx[A/G]WVRQAPGKGLE | SISSWLAWYQQKPGKAPKLLIYKASSL |
| | WVSx[A/T]ISGSGGLTYYADSVKGRFTI | ESGVPSRFSGSGSGTEFTLTISSLQPDD |
| | SRDNSKNTLYLQMNSLRAEDTAVYYC | FATYYCQQYKSYITFGGGTKVEIK |
| | AKAPYGYYMDVWGKGTTVTVSS | (SEQ ID NO: 762) |
| | (SEQ ID NO: 761) | |
| 25 | QVQLVQSGAEVKKPGASVKVSCKASG | DIQMTQSPSTLSASVGDRVTITCx[R/Q] |
| | YTFx[D/R]x[S/V/A]YGISWVRQAPGQG | ASx[Q/E/H]SIx[S/D/N]x[S/N]WLAW |
| | LEWMGWx[I/V]APYx[S/N]GNTNYAQK | YQQKPGKAPKLLIYx[K/S]Ax[S/Y]x |
| | LQGRVTMTTDTSTSTAYMELRSLRSD | [S/Y/N]LEx[S/Y]GVPSRFSGSGSGTEF |
| | DTAVYYCARDAGTYSPx[F/Y]GYGMD | TLTISSLQPDDFATYYCQx[Q/L/R]FQx |
| | VWGQGTTVTVSS (SEQ ID NO: 763) | [S/K]LPPFTFGGGTKVEIK |
| | | (SEQ ID NO: 764) |
| 29 | QVQLVESGGGVVQPGRSLRLSCAASG | DIVMTQSPDSLAVSLGERATINCKSSQ |
| | FTFx[H/R]Sx[R/Y]GMHWVRQAPGKGL | SVLFSSNNKNYLAWYQQKPGQPPKLL |
| | EWVAVITYDGINKYYADSVEGRFTISR | IYWASTRESGVPDRFSGSGSGTDFTLTI |
| | DNSKNTLYLQMNSLRAEDTAVYYCA | SSLQAEDVAVYYCQQFHSYPLTFGGG |
| | RDGVYYGVYDYWGQGTLVTVSS | TKVEIK (SEQ ID NO: 766) |
| | (SEQ ID NO: 765) | |
| 39 | QVQLVQSGAEVKKPGSSVKVSCKASG | EIVMTQSPATLSVSPGERATLSCRASQ |
| | GTFSSNAIGWVRQAPGQGLEWMGSIIP | SVSSNLAWYQQKPGQAPRLLIYGAST |
| | IIGFANYAQKFQGRVTITADESTSTAY | RATGIPARFSGSGSGTEFTLTISSLQSED |
| | MELSSLRSEDTAVYYCARDSGYYYGA | FAVYYCEQYNNLPLTFGGGTKVEIK |
| | SSFGMDVWGQGTTVTVSS (SEQ ID | (SEQ ID NO: 768) |
| | NO: 767) | |
| 43 | QVQLQx[E/Q]x[S/W]Gx[P/A]GLx[V/L] | EIVLTQSPGTLSLSPGERATLSCRASx[Q/ |
| | KPSx[Q/E]TLSLTCx[T/A]Vx[S/Y]GGSx | E/D]SVx[S/D]SSx[Y/F]LAWYQQKPGQ |
| | [I/L]SSGx[Q/Y]YWSWIRQx[H/P]PGKGLE | APRLLIYGAx[S/D/F/Y]x[S/T]Rx[A/Q] |
| | WIGEIx[Y/G]x[Y/A]SGSTRYNPSLKSRV | x[T/N]GIPDRFSGSGSGTDFTLTISRLEPE |
| | TISVDTSKx[N/D]QFSLKLSSVTAADTA | DFAVYYCQQx[V/A/D]GVVPYTFGGGT |
| | VYYCARDx[T/A]PYYYx[E/G/D]GGYY | KVEIK (SEQ ID NO: 770) |
| | YYMDVWGKGTTVTVSS (SEQ ID | |
| | NO: 769) | |
| 54 | QVQLVQSGAEVKKPGASVKVSCKASG | DIQMTQSPSSLSASVGDRVTITCQASQ |
| | YTFANYYMHWVRQAPGQGLEWMGII | DISNSLNWYQQKPGKAPKLLIYDASNL |
| | NPSGGITVYAQKFQGRVTMTRDTSTST | ETGVPSRFSGSRSGTDFTFTISSLQPEDI |
| | VYMELSSLRSEDTAVYYCARGGSKVA | ATYYCQQYNFHPLTFGGGTKVEIK |
| | ALAFDIWGQGTMVTVSS (SEQ ID | (SEQ ID NO: 772) |
| | NO: 771) | |

TABLE 15
Antibody 1F-CDR Sequences

| | | Exem- | | | | Con- | |
|---|---|---|---|---|---|---|---|
| | | plary* | Kabat | Chothia | AbM | tact | IMGT |
| VH | VH | GFTFSD | DYAMG | GFTFSDY | GFTFS | SDYAMG | GFTF |
| CDR | CDR1 | YAMG | (SEQ | (SEQ ID | DYAMG | (SEQ | SDYA |
| Seq. | | (SEQ | ID NO: | NO: 13) | (SEQ | ID NO: | (SEQ |
| | | ID NO: | 7) | | ID | 25) | ID |
| | | 1) | | | NO: | | NO: |
| | | | | | 19) | | 31) |
| | VH | TISGSG | TISGSG | GSGG | TISGS | WVSTIS | ISGS |
| | CDR2 | GLTYYA | GLTYYA | (SEQ ID | GGLTY | GSGGLT | GGLT |
| | | DSVKG | DSVKG | NO: 14) | (SEQ | Y | (SEQ |
| | | (SEQ | (SEQ | | ID | (SEQ | ID |
| | | ID NO: | ID NO: | | NO: | ID NO: | NO: |
| | | 2) | 8) | | 20) | 26) | 32) |
| | VH | APYGYY | APYGYY | PYGYYMD | APYGY | AKAPYG | AKAP |
| | CDR3 | MDV | MDV | (SEQ ID | YMDV | YYMD | YGYY |
| | | (SEQ | (SEQ | NO: 15) | (SEQ | (SEQ | MDV |
| | | ID NO: | ID NO: | | ID | ID NO: | (SEQ |
| | | 3) | 9) | | NO: | 27) | ID |
| | | | | | 21) | | NO: |
| | | | | | | | 33) |
| VL | VL | RASQSI | RASQSI | SQSISSW | RASQS | SSWLAW | QSIS |
| CDR | CDR1 | SSWLA | SSWLA | (SEQ ID | ISSWL | Y | SW |
| Seq. | | (SEQ | (SEQ | NO: 16) | A | (SEQ | (SEQ |
| | | ID NO: | ID NO: | | (SEQ | ID NO: | ID |
| | | 4) | 10) | | ID | 28) | NO: |
| | | | | | NO: | | 34) |
| | | | | | 22) | | |
| | VL | KASSLE | KASSLE | KAS | KASSL | LLIYKA | KAS |
| | CDR2 | S | S | (SEQ ID | ES | SSLE | (SEQ |
| | | (SEQ | (SEQ | NO: 17) | (SEQ | (SEQ | ID |
| | | ID NO: | ID NO: | | ID | ID NO: | NO: |
| | | 5) | 11) | | NO: | 29) | 35) |
| | | | | | 23) | | |
| | VL | QQYKSY | QQYKS | YKSYI | QQYKS | QQYKSY | QQYK |
| | CDR3 | IT | YIT | (SEQ ID | YIT | I | SYIT |
| | | (SEQ | (SEQ | NO: 18) | (SEQ | (SEQ | (SEQ |
| | | ID NO: | ID NO: | | ID | ID NO: | ID |
| | | 6) | 12) | | NO: | 30) | NO: |
| | | | | | 24) | | 36) |

VH Sequence*:
EVQLLESGGGLVQPGGSLRLSCAASGFTFSDYAMGWVRQAPGKGLEWVSTISGSGGLTYYADSVKGRFTISRDNSKNTLYLQMNSLRAEDTAVYYCAKAPYGYYMDVWGKGTTVTVSS (SEQ ID NO: 37)
VL Sequence*:
DIQMTQSPSTLSASVGDRVTITCRASQSISSWLAWYQQKPGKAPKLLIYKASSLESGVPSRFSGSGSGTEFTLTISSLQPDDFATYYCQQYKSYITFGGGTKVEIK (SEQ ID NO: 38)
*Exemplary CDR sequences encompass amino acids as determined by Kabat plus Chothia

TABLE 16
Antibody 1G-CDR Sequences

| | | Exem- | | | | Con- | |
|---|---|---|---|---|---|---|---|
| | | plary* | Kabat | Chothia | AbM | tact | IMGT |
| VH | VH | GFTFSS | SYAMA | GFTFSSY | GFTFS | SSYAMA | GFTFS |
| CDR | CDR1 | YAMA | (SEQ | (SEQ ID | SYAMA | (SEQ | SYA |
| Seq. | | (SEQ | ID NO: | NO: 51) | (SEQ | ID NO: | (SEQ |
| | | ID NO: | 45) | | ID | 63) | ID |
| | | 39) | | | NO: | | NO: |
| | | | | | 57) | | 69) |
| | VH | AISGSG | AISGSG | GSGG | AISGS | WVSAIS | ISGSG |
| | CDR2 | GLTYYA | GLTYYA | (SEQ ID | GGLTY | GSGGLT | GLT |
| | | DSVKG | DSVKG | NO: 52) | (SEQ | Y | (SEQ |
| | | (SEQ | (SEQ | | ID | (SEQ | ID |
| | | ID NO: | ID NO: | | NO: | ID NO: | NO: |
| | | 40) | 46) | | 58) | 64) | 70) |
| | VH | APYGYY | APYGYY | PYGYYMD | APYGY | AKAPYG | AKAPY |
| | CDR3 | MDV | MDV | (SEQ ID | YMDV | YYMD | GYYMD |
| | | (SEQ | (SEQ | NO: 53) | (SEQ | (SEQ | V |
| | | ID NO: | ID NO: | | ID | ID NO: | (SEQ |
| | | 41) | 47) | | NO: | 65) | ID |
| | | | | | 59) | | NO: |
| | | | | | | | 71) |
| VL | VL | RASQSI | RASQSI | SQSISSW | RASQS | SSWLAW | QSISS |
| CDR | CDR1 | SSWLA | SSWLA | (SEQ ID | ISSWL | Y | W |
| Seq. | | (SEQ | (SEQ | NO: 54) | A | (SEQ | (SEQ |
| | | ID NO: | ID NO: | | (SEQ | ID NO: | ID |
| | | 42) | 48) | | ID | 66) | NO: |
| | | | | | NO: | | 72) |
| | | | | | 60) | | |
| | VL | KASSLE | KASSLE | KAS | KASSL | LLIYKA | KAS |
| | CDR2 | S | S | (SEQ ID | ES | SSLE | (SEQ |
| | | (SEQ | (SEQ | NO: 55) | (SEQ | (SEQ | ID |
| | | ID NO: | ID NO: | | ID | ID NO: | NO: |
| | | 43) | 49) | | NO: | 67) | 73) |
| | | | | | 61) | | |
| | VL | QQYKSY | QQYKSY | YKSYI | QQYKS | QQYKSY | QQYKS |
| | CDR3 | IT | IT | (SEQ ID | YIT | I | YIT |
| | | (SEQ | (SEQ | NO: 56) | (SEQ | (SEQ | (SEQ |
| | | ID NO: | ID NO: | | ID | ID NO: | ID |
| | | 44) | 50) | | NO: | 68) | NO: |
| | | | | | 62) | | 74) |

VH Sequence*:
EVQLLESGGGLVQPGGSLRLSCAASGFTFSSYAMAWVRQAPGKGLEWVSAISGSGGLTYYADSVKGRFTISRDNSKNTLYLQMNSLRAEDTAVYYCAKAPYGYYMDVWGKGTTVTVSS (SEQ ID NO: 75)
VL Sequence*:
DIQMTQSPSTLSASVGDRVTITCRASQSISSWLAWYQQKPGKAPKLLIYKASSLESGVPSRFSGSGSGTEFTLTISSLQPDDFATYYCQQYKSYITFGGGTKVEIK (SEQ ID NO: 76)
*Exemplary CDR sequences encompass amino acids as determined by Kabat plus Chothia

TABLE 17
Antibody 25A-CDR Sequences

| | | Exem- | | | | Con- | |
|---|---|---|---|---|---|---|---|
| | | plary* | Kabat | Chothia | AbM | tact | IMGT |
| VH | VH | GYTFDV | VYGIS | GYTFDVY | GYTFD | DVYGIS | GYTFD |
| CDR | CDR1 | YGIS | (SEQ | (SEQ ID | VYGIS | (SEQ | VYG |
| Seq. | | (SEQ | ID NO: | NO: 89) | (SEQ | ID NO: | (SEQ |
| | | ID NO: | 83) | | ID | 101) | ID |
| | | 77) | | | NO: | | NO: |
| | | | | | 95) | | 107) |
| | | WIAPYN | WIAPYN | PYNG | WIAPY | WMGWIA | IAPYN |
| | VH | GNTNY | GNTNY | (SEQ ID | NGNTN | PYNGN | GNT |
| | CDR2 | AQKLQG | AQKLQG | NO: 90) | (SEQ | TN | (SEQ |
| | | (SEQ | (SEQ | | ID | (SEQ | ID |
| | | ID NO: | ID NO: | | NO: | ID NO: | NO: |
| | | 78) | 84) | | 96) | 102) | 108) |
| | VH | DAGTYS | DAGTYS | AGTYSPF | DAGTY | ARDAGT | ARDAG |
| | CDR3 | PFGYG | PFGYG | GYGM | SPFGY | YSPFGY | TYSPF |
| | | MDV | MDV | D | GMDV | GMD | GTGMD |
| | | (SEQ | (SEQ | (SEQ ID | (SEQ | (SEQ | V |
| | | ID NO: | ID NO: | NO: 91) | ID | ID NO: | (SEQ |
| | | 79) | 85) | | NO: | 103) | ID |
| | | | | | 97) | | NO: |
| | | | | | | | 109) |
| VL | VL | RASQSI | RASQSI | SQSISS | RASQS | SSWLAW | QSISS |
| CDR | CDR1 | SSWLA | SSWLA | W | ISSWL | Y | W |
| Seq. | | (SEQ | (SEQ | (SEQ ID | A | (SEQ | (SEQ |
| | | ID NO: | ID NO: | NO: 92) | (SEQ | ID NO: | ID |
| | | 80) | 86) | | ID | 104) | NO: |
| | | | | | NO: | | 110) |
| | | | | | 98) | | |
| | VL | KASSLE | KASSLE | KAS | KASSL | LLIYKA | KAS |
| | CDR2 | S | S | (SEQ ID | ES | SSLE | (SEQ |
| | | (SEQ | (SEQ | NO: 93) | (SEQ | (SEQ | ID |
| | | ID NO: | ID NO: | | ID | ID NO: | NO: |
| | | 81) | 87) | | NO: | 105) | 111) |
| | | | | | 99) | | |
| | VL | QQFQSL | QQFQSL | FQSLPP | QQFQS | QQFQSL | QQFQS |
| | CDR3 | PPFT | PPFT | F | LPPFT | PPF | LPPFT |
| | | (SEQ | (SEQ | (SEQ ID | (SEQ | (SEQ | (SEQ |
| | | ID NO: | ID NO: | NO: 94) | ID | ID NO: | ID |
| | | 82) | 88) | | NO: | 106) | NO: |
| | | | | | 100) | | 112) |

VH Sequence*:
QVQLVQSGAEVKKPGASVKVSCKASGYTFDVYGISWVRQAPGQGLEWMGWIAPYNGNTNYAQKLQGRVTMTTDTSTSTAYMELRSLRSDDTAVYYCARDAGTYSPFGYGMDVWGQGTTVTVSS (SEQ ID NO: 113)
VL Sequence*:
DIQMTQSPSTLSASVGDRVTITCRASQSISSWLAWYQQKPGKAPKLLIYKASSLESGVPSRFSGSGSGTEFTLTISSLQPDDFATYYCQQFQSLPPFTFGGGTKVEIK (SEQ ID NO: 114)
*Exemplary CDR sequences encompass amino acids as determined by Kabat plus Chothia

TABLE 18
Antibody 25A3-CDR Sequences

| | | Exem- | | Cho- | | Con- | |
|---|---|---|---|---|---|---|---|
| | | plary* | Kabat | thia | AbM | tact | IMGT |
| VH | VH | GYTFDV | VYGIS | GYTF | GYTFDV | DVYGIS | GYTFD |
| CDR | CDR1 | YGIS | (SEQ | DVY | YGIS | (SEQ | VYG |
| Seq. | | (SEQ | ID NO: | (SEQ | (SEQ | ID NO: | (SEQ |
| | | ID NO: | 121) | ID | ID NO: | 139) | ID |
| | | 115) | | NO: | 133) | | NO: |
| | | | | 127) | | | 145) |
| | VH | WIAPYS | WIAPYS | PYSG | WIAPYS | WMGWIA | IAPYS |
| | CDR2 | GNTNYA | GNTNYA | (SEQ | GNTN | PYSGN | GNT |
| | | QKLQG | QKLQG | ID | (SEQ | TN | (SEQ |
| | | (SEQ | (SEQ | NO: | ID NO: | (SEQ | ID |
| | | ID NO: | ID NO: | 128) | 134) | ID NO: | NO: |
| | | 116) | 122) | | | 140) | 146) |
| | VH | DAGTYS | DAGTYS | AGTY | DAGTYS | ARDAGT | ARDAG |
| | CDR3 | PFGYG | PFGYG | SPFD | PFGYG | YSPFGY | TYSPF |
| | | MDV | MDV | GYGM | MDV | GMD | GTGMD |
| | | (SEQ | (SEQ | (SEQ | (SEQ | (SEQ | V |
| | | ID NO: | ID | ID | ID | ID NO: | (SEQ |
| | | 117) | NO: | NO: | NO: | 141) | ID |
| | | | 123) | 129) | 135) | | NO: |
| | | | | | | | 147) |
| VL | VL | QASQSI | QASQSI | SQSI | QASQSI | NNWLAW | QSINN |
| CDR | CDR1 | NNWLA | NNWLA | NNW | NNWLA | Y | W |
| Seq. | | (SEQ | (SEQ | (SEQ | (SEQ | (SEQ | (SEQ |
| | | ID NO: | ID NO: | ID | ID NO: | ID NO: | ID |
| | | 118) | 124) | NO: | 136) | 142) | NO: |
| | | | | 130) | | | 148) |
| | VL | KAYNLE | KAYNLE | KAY | KAYNLE | LLIYKA | KAY |
| | CDR2 | S | S | (SEQ | S | YNLE | (SEQ |
| | | (SEQ | (SEQ | ID | (SEQ | (SEQ | ID |
| | | ID | ID | NO: | ID | ID | NO: |
| | | NO: | NO: | 131) | NO: | NO: | 149) |
| | | 119) | 125) | | 137) | 143) | |
| | VL | QLFQSL | QLFQSL | FQSL | QLFQSL | QLFQSL | QLFQS |
| | CDR3 | PPFT | PPFT | PPF | PPFT | PPF | LPPFT |
| | | (SEQ | (SEQ | (SEQ | (SEQ | (SEQ | (SEQ |
| | | ID | ID | ID | ID | ID | ID |
| | | NO: | NO: | NO: | NO: | NO: | NO: |
| | | 120) | 126) | 132) | 138) | 144) | 150) |

VH Sequence*:
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSS (SEQ ID NO: 151)
VL Sequence*:
DIQMTQSPSTLSASVGDRVTITCQASQSINNWLAWYQQKPGKAPKLLIYKAYNLESGVPSRFSGSGSGTEFTLTISSLQPDDFATYYCQLFQSLPPFTFGGGTKVEIK (SEQ ID NO: 152)
*Exemplary CDR sequences encompass amino acids as determined by Kabat plus Chothia

TABLE 19a
Antibody 25A5-CDR Sequences

| | | Exem- | | Cho- | | Con | |
|---|---|---|---|---|---|---|---|
| | | plary* | Kabat | thia | AbM | tact | IMGT |
| VH | VH | GYTFDV | VYGIS | GYTFDV | GYTFDV | DVYGIS | GYTF |
| CDR | CDR1 | YGIS | (SEQ | Y | YGIS | (SEQ | DVYG |
| Seq. | | (SEQ | ID | (SEQ | (SEQ | ID | (SEQ |
| | | ID | NO: | ID | ID | NO: | ID |
| | | NO: | 159) | NO: | NO: | 177) | NO: |
| | | 153) | | 165) | 171) | | 183) |
| | VH | WIAPYS | WIAPYS | PYSG | WIAPYS | WMGWIA | IAPY |
| | CDR2 | GNTNYA | GNTNYA | (SEQ | GNTN | PYSGN | SGNT |
| | | QKLQG | QKLQG | ID | (SEQ | TN | (SEQ |
| | | (SEQ | (SEQ | NO: | ID | (SEQ | ID |
| | | ID | ID | 166) | NO: | ID | NO: |
| | | NO: | NO: | | 172) | NO: | 184) |
| | | 154) | 160) | | | 178) | |
| | VH | DAGTYS | DAGTYS | AGTYSP | DAGTYS | ARDAGT | ARDA |
| | CDR3 | PFGYG | PFGYG | FGYGM | PFGYG | YSPFGY | GTYS |
| | | MDV | MDV | D | MDV | GMD | PFGT |
| | | (SEQ | (SEQ | (SEQ | (SEQ | (SEQ | GMDV |
| | | ID | ID | ID | ID | ID | (SEQ |
| | | NO: | NO: | NO: | NO: | NO: | ID |
| | | 155) | 161) | 167) | 173) | 179) | NO: |
| | | | | | | | 185) |
| VL | VL | RASESI | RASESI | SESISN | RASESI | SNWLAW | ESIS |
| CDR | CDR1 | SNWLA | SNWLA | W | SNWLA | Y | NW |
| Seq. | | (SEQ | (SEQ | (SEQ | (SEQ | (SEQ | (SEQ |
| | | ID | ID | ID | ID | ID | ID |
| | | NO: | NO: | NO: | NO: | NO: | NO: |
| | | 156) | 162) | 168) | 174) | 180) | 186) |
| | VL | KAYSLE | KAYSLE | KAY | KAYSLE | LLIYKA | KAY |
| | CDR2 | Y | Y | (SEQ | Y | YSLE | (SEQ |
| | | (SEQ | (SEQ | ID | (SEQ | (SEQ | ID |
| | | ID | ID | NO: | ID | ID | NO: |
| | | NO: | NO: | 169) | NO: | NO: | 187) |
| | | 157) | 163) | | 175) | 181) | |
| | VL | QQFQKL | QQFQKL | FQKLPP | QQFQKL | QQFQKL | QQFQ |
| | CDR3 | PPFT | PPFT | F | PPFT | PPF | KLPP |
| | | (SEQ | (SEQ | (SEQ | (SEQ | (SEQ | FT |
| | | ID | ID | ID | ID | ID | (SEQ |
| | | NO: | NO: | NO: | NO: | NO: | ID |
| | | 158) | 164) | 170) | 176) | 182) | NO: |
| | | | | | | | 188) |

VH Sequence*:
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSS (SEQ ID NO: 189)
VL Sequence*:
DIQMTQSPSTLSASVGDRVTITCRASESISNWLAWYQQKPGKAPKLLIYKAYSLEYGVPSRFSGSGSGTEFTLTISSLQPDDFATYYCQQFQKLPPFTFGGGTKVEIK (SEQ ID NO: 190)
*Exemplary CDR sequences encompass amino acids as determined by Kabat plus Chothia

TABLE 19b
Antibody 25A5-T-CDR Sequences

| | | Exem- | | Cho- | | Con- | |
|---|---|---|---|---|---|---|---|
| | | plary* | Kabat | thia | AbM | tact | IMGT |
| VH | VH | GYTFDA | AYGIS | GYTFDA | GYTFDA | DAYGIS | GYTF |
| CDR | CDR1 | YGIS | (SEQ | Y | YGIS | (SEQ | DAYG |
| Seq. | | (SEQ | ID | (SEQ | (SEQ | ID | (SEQ |
| | | ID | NO: | ID | ID | NO: | ID |
| | | NO: | 890) | NO: | NO: | 908) | NO: |
| | | 884) | | 896) | 902) | | 914) |
| | VH | WIAPYS | WIAPYS | PYSG | WIAPYS | WMGWIA | IAPY |
| | CDR2 | GNTNYA | GNTNYA | (SEQ | GNTN | PYSGN | SGNT |
| | | QKLQG | QKLQG | ID | (SEQ | TN | (SEQ |
| | | (SEQ | (SEQ | NO: | ID | (SEQ | ID |
| | | ID | ID | 897) | NO: | ID | NO: |
| | | NO: | NO: | | 903) | NO: | 915) |
| | | 885) | 891) | | | 909) | |
| | VH | DAGTYS | DAGTYS | AGTYSP | DAGTYS | ARDAGT | ARDA |
| | CDR3 | PFGYG | PFGYG | FGYGM | PFGYG | YSPFGY | GTYS |
| | | MDV | MDV | D | MDV | GMD | PFGT |
| | | (SEQ | (SEQ | (SEQ | (SEQ | (SEQ | GMDV |
| | | ID | ID | ID | ID | ID | (SEQ |
| | | NO: | NO: | NO: | NO: | NO: | ID |
| | | 886) | 892) | 898) | 904) | 910) | NO: |
| | | | | | | | 916) |
| VL | VL | RASESI | RASESI | SESISN | RASESI | SNWLAW | ESIS |
| CDR | CDR1 | SNWLA | SNWLA | W | SNWLA | Y | NW |
| Seq. | | (SEQ | (SEQ | (SEQ | (SEQ | (SEQ | (SEQ |
| | | ID | ID | ID | ID | ID | ID |
| | | NO: | NO: | NO: | NO: | NO: | NO: |
| | | 887) | 893) | 899) | 905) | 911) | 917) |
| | VL | KAYSLE | KAYSLE | KAY | KAYSLE | LLIYKA | KAY |
| | CDR2 | Y | Y | (SEQ | Y | YSLE | (SEQ |
| | | (SEQ | (SEQ | ID | (SEQ | (SEQ | ID |
| | | ID | ID | NO: | ID | ID | NO: |
| | | NO: | NO: | 900) | NO: | NO: | 918) |
| | | 888) | 894) | | 906) | 912) | |
| | VL | QQFQKL | QQFQKL | FQKLPP | QQFQKL | QQFQKL | QQFQ |
| | CDR3 | PPFT | PPFT | F | PPFT | PPF | KLPP |
| | | (SEQ | (SEQ | (SEQ | (SEQ | (SEQ | FT |
| | | ID | ID | ID | ID | ID | (SEQ |
| | | NO: | NO: | NO: | NO: | NO: | ID |
| | | 889) | 895) | 901) | 907) | 913) | NO: |
| | | | | | | | 919) |

VH Sequence*:
QVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSS (SEQ ID NO: 836)
VL Sequence*:
DIQMTQSPSTLSASVGDRVTITCRASESISNWLAWYQQKPGKAPKLLIYKAYSLEYGVPSRFSGSGSGTEFTLTISSLQPDDFATYYCQQFQKLPPFTFGGGTKVEIK (SEQ ID NO: 837)
*Exemplary CDR sequences encompass amino acids as determined by Kabat plus Chothia

TABLE 20
Antibody 25G-CDR Sequences

| | | Exem- | | Cho- | | Con- | |
|---|---|---|---|---|---|---|---|
| | | plary* | Kabat | thia | AbM | tact | IMGT |
| VH | VH | GYTFRS | SYGIS | GYTFRS | GYTFRS | RSYGIS | GYTF |
| CDR | CDR1 | YGIS | (SEQ | Y | YGIS | (SEQ | RSYG |
| Seq. | | (SEQ | ID | (SEQ | (SEQ | ID | (SEQ |
| | | ID | NO: | ID | ID | NO: | ID |
| | | NO: | 197) | NO: | NO: | 215) | NO: |
| | | 191) | | 203) | 209) | | 221) |
| | VH | WVAPYN | WVAPYN | PYNG | WVAPYN | WMGWVA | VAPY |
| | CDR2 | GNTNYA | GNTNY | (SEQ | GNTN | PYNG | NGNT |
| | | QKLQG | AQKLQG | ID | (SEQ | NTN | (SEQ |
| | | (SEQ | (SEQ | NO: | ID | (SEQ | ID |
| | | ID | ID | 204) | NO: | ID | NO: |
| | | NO: | NO: | | 210) | NO: | 222) |
| | | 192) | 198) | | | 216) | |
| | VH | DAGTYS | DAGTYS | AGTYSP | DAGTYS | ARDAGT | ARDA |
| | CDR3 | PYGYG | PYGYG | YGYGM | PYGYG | YSPYG | GTYS |
| | | MDV | MDV | D | MDV | YGMD | PYGY |
| | | (SEQ | (SEQ | (SEQ | (SEQ | (SEQ | GMDV |
| | | ID | ID | ID | ID | ID | (SEQ |
| | | NO: | NO: | NO: | NO: | NO: | ID |
| | | 193) | 199) | 205) | 211) | 217) | NO: |
| | | | | | | | 223) |
| VL | VL | RASQSI | RASQSI | SQSISS | RASQSI | SSWLAW | QSIS |
| CDR | CDR1 | SSWLA | SSWLA | W | SSWLA | Y | SW |
| Seq. | | (SEQ | (SEQ | (SEQ | (SEQ | (SEQ | (SEQ |
| | | ID | ID | ID | ID | ID | ID |
| | | NO: | NO: | NO: | NO: | NO: | NO: |
| | | 194) | 200) | 206) | 212) | 218) | 224) |
| | VL | KASSLE | KASSLE | KAS | KASSLE | LLIYKA | KAS |
| | CDR2 | S | S | (SEQ | S | SSLE | (SEQ |
| | | (SEQ | (SEQ | ID | (SEQ | (SEQ | ID |
| | | ID | ID | NO: | ID | ID | NO: |
| | | NO: | NO: | 207) | NO: | NO: | 225) |
| | | 195) | 201) | | 213) | 219) | |
| | VL | QQFQSL | QQFQSL | FQSLPP | QQFQSL | QQFQSL | QQFQ |
| | CDR3 | PPFT | PPFT | F | PPFT | PPF | SLPP |
| | | (SEQ | (SEQ | (SEQ | (SEQ | (SEQ | FT |
| | | ID | ID | ID | ID | ID | (SEQ |
| | | NO: | NO: | NO: | NO: | NO: | ID |
| | | 196) | 202) | 208) | 214) | 220) | NO: |
| | | | | | | | 226) |

VH Sequence*:
QVQLVQSGAEVKKPGASVKVSCKASGYTFRSYGISWVRQAPGQGLEWMGWVAPYNGNTNYAQKLQGRVTMTTDTSTSTAYMELRSLRSDDTAVYYCARDAGTYSPYGYGMDVWGQGTTVTVSS (SEQ ID NO: 227)
VL Sequence*:
DIQMTQSPSTLSASVGDRVTITCRASQSISSWLAWYQQKPGKAPKLLIYKASSLESGVPSRFSGSGSGTEFTLTISSLQPDDFATYYCQQFQSLPPFTFGGGTKVEIK (SEQ ID NO: 228)
*Exemplary CDR sequences encompass amino acids as determined by Kabat plus Chothia

TABLE 21
Antibody 25G1-CDR Sequences

| | | Exem- | | Cho- | | Con- | |
|---|---|---|---|---|---|---|---|
| | | plary* | Kabat | thia | AbM | tact | IMGT |
| VH | VH | GYTFRS | SYGIS | GYTFRS | GYTFRS | RSYGIS | GYTF |
| CDR | CDR1 | YGIS | (SEQ | Y | YGIS | (SEQ | RSYG |
| Seq. | | (SEQ | ID | (SEQ | (SEQ | ID | (SEQ |
| | | ID | NO: | ID | ID | NO: | ID |
| | | NO: | 235) | NO: | NO: | 253) | NO: |
| | | 229) | | 241) | 247) | | 259) |
| | VH | WVAPYS | WVAPYS | PYSG | WVAPYS | WMGWVA | VAPY |
| | CDR2 | GNTNY | GNTNY | (SEQ | GNTN | PYSG | SGNT |
| | | AQKLQG | AQKLQG | ID | (SEQ | NTN | (SEQ |
| | | (SEQ | (SEQ | NO: | ID | (SEQ | ID |
| | | ID | ID | 242) | NO: | ID | NO: |
| | | NO: | NO: | | 248) | NO: | 260) |
| | | 230) | 236) | | | 254) | |
| | VH | DAGTYS | DAGTYS | AGTYSP | DAGTYS | ARDAGT | ARDA |
| | CDR3 | PYGYG | PYGYG | YGYGM | PYGYG | YSPYG | GTYS |
| | | MDV | MDV | D | MDV | YGMD | PYGY |
| | | (SEQ | (SEQ | (SEQ | (SEQ | (SEQ | GMDV |
| | | ID | ID | ID | ID | ID | (SEQ |
| | | NO: | NO: | NO: | NO: | NO: | ID |
| | | 231) | 237) | 243) | 249) | 255) | NO: |
| | | | | | | | 261) |
| VL | VL | RASHSI | RASHSI | SHSIDS | RASHSI | DSWLAW | HSID |
| CDR | CDR1 | DSWLA | DSWLA | W | DSWLA | Y | SW |
| Seq. | | (SEQ | (SEQ | (SEQ | (SEQ | (SEQ | (SEQ |
| | | ID | ID | ID | ID | ID | ID |
| | | NO: | NO: | NO: | NO: | NO: | NO: |
| | | 232) | 238) | 244) | 250) | 256) | 262) |
| | VL | KASYLE | KASYLE | KAS | KASYLE | LLIYKA | KAS |
| | CDR2 | S | S | (SEQ | S | SYLE | (SEQ |
| | | (SEQ | (SEQ | ID | (SEQ | (SEQ | ID |
| | | ID | ID | NO: | ID | ID | NO: |
| | | NO: | NO: | 245) | NO: | NO: | 263) |
| | | 233) | 239) | | 251) | 257) | |
| | VL | QLFQSL | QLFQSL | FQSLPP | QLFQSL | QLFQSL | QLFQ |
| | CDR3 | PPFT | PPFT | F | PPFT | PPF | SLPP |
| | | (SEQ | (SEQ | (SEQ | (SEQ | (SEQ | FT |
| | | ID | ID | ID | ID | ID | (SEQ |
| | | NO: | NO: | NO: | NO: | NO: | ID |
| | | 234) | 240) | 246) | 252) | 258) | NO: |
| | | | | | | | 264) |

VH Sequence*:
QVQLVQSGAEVKKPGASVKVSCKASGYTFRSYGISWVRQAPGQGLEWMGWVAPYSGNTNYAQKLQGRVTMTTDTSTSTAYMELRSLRSDDTAVYYCARDAGTYSPYGYGMDVWGQGTTVTVSS (SEQ ID NO: 265)
VL Sequence*:
DIQMTQSPSTLSASVGDRVTITCRASHSIDSWLAWYQQKPGKAPKLLIYKASYLESGVPSRFSGSGSGTEFTLTISSLQPDDFATYYCQLFQSLPPFTFGGGTKVEIK (SEQ ID NO: 266)
*Exemplary CDR sequences encompass amino acids as determined by Kabat plus Chothia

TABLE 22
Antibody 25G9-CDR Sequences

| | | Exem- | | Cho- | | Con- | |
|---|---|---|---|---|---|---|---|
| | | plary* | Kabat | thia | AbM | tact | IMGT |
| VH | VH | GYTFRS | SYGIS | GYTFRS | GYTFRS | RSYGIS | GYTF |
| CDR | CDR1 | YGIS | (SEQ | Y | YGIS | (SEQ | RSYG |
| Seq. | | (SEQ | ID | (SEQ | (SEQ | ID | (SEQ |
| | | ID | NO: | ID | ID | NO: | ID |
| | | NO: | 273) | NO: | NO: | 291) | NO: |
| | | 267) | | 279) | 285) | | 297) |
| | VH | WVAPYS | WVAPYS | PYSG | WVAPYS | WMGWVA | VAPY |
| | CDR2 | GNTNYA | GNTNY | (SEQ | GNTN | PYSG | SGNT |
| | | QKLQG | AQKLQG | ID | (SEQ | NTN | (SEQ |
| | | (SEQ | (SEQ | NO: | ID | (SEQ | ID |
| | | ID | ID | 280) | NO: | ID | NO: |
| | | NO: | NO: | | 286) | NO: | 298) |
| | | 268) | 274) | | | 292) | |
| | VH | DAGTYS | DAGTYS | AGTYSP | DAGTYS | ARDAGT | ARDA |
| | CDR3 | PYGY | PYGYG | YGYGMD | PYGYG | YSPYG | GTYS |
| | | GMDV | MDV | (SEQ | MDV | YGMD | PYGY |
| | | (SEQ | (SEQ | ID | (SEQ | (SEQ | GMDV |
| | | ID | ID | NO: | ID | ID | (SEQ |
| | | NO: | NO: | 281) | NO: | NO: | ID |
| | | 269) | 275) | | 287) | 293) | NO: |
| | | | | | | | 299) |
| VL | VL | QASQSI | QASQSI | SQSIDS | QASQSI | DSWLAW | QSID |
| CDR | CDR1 | DSWLA | DSWLA | W | DSWLA | Y | SW |
| Seq. | | (SEQ | (SEQ | (SEQ | (SEQ | (SEQ | (SEQ |
| | | ID | ID | ID | ID | ID | ID |
| | | NO: | NO: | NO: | NO: | NO: | NO: |
| | | 270) | 276) | 282) | 288) | 294) | 300) |
| | VL | SASYLE | SASYLE | SAS | SASYLE | LLIYSA | SAS |
| | CDR2 | S | S | (SEQ | S | SYLE | (SEQ |
| | | (SEQ | (SEQ | ID | (SEQ | (SEQ | ID |
| | | ID | ID | NO: | ID | ID | NO: |
| | | NO: | NO: | 283) | NO: | NO: | 301) |
| | | 271) | 277) | | 289) | 295) | |
| | VL | QRFQSL | QRFQSL | FQSLPP | QRFQSL | QRFQSL | QRFQ |
| | CDR3 | PPFT | PPFT | F | PPFT | PPF | SLPP |
| | | (SEQ | (SEQ | (SEQ | (SEQ | (SEQ | FT |
| | | ID | ID | ID | ID | ID | (SEQ |
| | | NO: | NO: | NO: | NO: | NO: | ID |
| | | 272) | 278) | 284) | 290) | 296) | NO: |
| | | | | | | | 302) |

VH Sequence*:
QVQLVQSGAEVKKPGASVKVSCKASGYTFRSYGISWVRQAPGQGLEWMGWVAPYSGNTNYAQKLQGRVTMTTDTSTSTAYMELRSLRSDDTAVYYCARDAGTYSPYGYGMDVWGQGTTVTVSS (SEQ ID NO: 303)
VL Sequence*:
DIQMTQSPSTLSASVGDRVTITCQASQSIDSWLAWYQQKPGKAPKLLIYSASYLESGVPSRFSGSGSGTEFTLTISSLQPDDFATYYCQRFQSLPPFTFGGGTKVEIK (SEQ ID NO: 304)
*Exemplary CDR sequences encompass amino acids as determined by Kabat plus Chothia

TABLE 23
Antibody 29D-CDR Sequences

| | | Exem- | | Cho- | | Con- | |
|---|---|---|---|---|---|---|---|
| | | plary* | Kabat | thia | AbM | tact | IMGT |
| VH | VH | GFTFHS | SRGMH | GFTFHS | GFTFHS | HSRGMH | GFTF |
| CDR | CDR1 | RGMH | (SEQ | R | RGMH | (SEQ | HSRG |
| Seq. | | (SEQ | ID | (SEQ | (SEQ | ID | (SEQ |
| | | ID | NO: | ID | ID | NO: | ID |
| | | NO: | 311) | NO: | NO: | 329) | NO: |
| | | 305) | | 317) | 323) | | 335) |
| | VH | VITYDG | VITYDG | YDGI | VITYDG | WVAVIT | ITYD |
| | CDR2 | INKYYA | INKYYA | (SEQ | INKY | YDGINK | GINK |
| | | DSVEG | DSVEG | ID | (SEQ | Y | (SEQ |
| | | (SEQ | (SEQ | NO: | ID | (SEQ | ID |
| | | ID | ID | 318) | NO: | ID | NO: |
| | | NO: | NO: | | 324) | NO: | 336) |
| | | 306) | 312) | | | 330) | |
| | VH | DGVYYG | DGVYYG | GVYYGV | DGVYYG | ARDGVY | ARDG |
| | CDR3 | VYDY | VYDY | YD | VYDY | YGVYD | VYYG |
| | | (SEQ | (SEQ | (SEQ | (SEQ | (SEQ | VYDY |
| | | ID | ID | ID | ID | ID | (SEQ |
| | | NO: | NO: | NO: | NO: | NO: | ID |
| | | 307) | 313) | 319) | 325) | 331) | NO: |
| | | | | | | | 337) |
| VL | VL | KSSQSV | KSSQSV | SQSVLF | KSSQSV | LFSSNN | QSVL |
| CDR | CDR1 | LFSSNN | LFSSNN | SSNNKN | LFSSNN | KNYLA | FSSN |
| Seq. | | KNYLA | KNYLA | Y | KNYLA | WY | NKNY |
| | | (SEQ | (SEQ | (SEQ | (SEQ | (SEQ | (SEQ |
| | | ID | ID | ID | ID | ID | ID |
| | | NO: | NO: | NO: | NO: | NO: | NO: |
| | | 308) | 314) | 320) | 326) | 332) | 338) |
| | VL | WASTRE | WASTRE | WAS | WASTRE | LLIYWA | WAS |
| | CDR2 | S | S | (SEQ | S | STRE | (SEQ |
| | | (SEQ | (SEQ | ID | (SEQ | (SEQ | ID |
| | | ID | ID | NO: | ID | ID | NO: |
| | | NO: | NO: | 321) | NO: | NO: | 339) |
| | | 309) | 315) | | 327) | 333) | |
| | VL | QQFHSY | QQFHSY | FHSYPL | QQFHSY | QQFHSY | QQFHSY |
| | CDR3 | PLT | PLT | (SEQ | PLT | PL | PLT |
| | | (SEQ | (SEQ | ID | (SEQ | (SEQ | (SEQ |
| | | ID | ID | NO: | ID | ID | ID |
| | | NO: | NO: | 322) | NO: | NO: | NO: |
| | | 310) | 316) | | 328) | 334) | 340) |

VH Sequence*:
QVQLVESGGGVVQPGRSLRLSCAASGFTFHSRGMHWVRQAPGKGLEWVAVITYDGINKYYADSVEGRFTISRDNSKNTLYLQMNSLRAEDTAVYYCARDGVYYGVYDYWGQGTLVTVSS (SEQ ID NO: 341)
VL Sequence*:
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQFHSYPLTFGGGTKVEIK (SEQ ID NO: 342)
*Exemplary CDR sequences encompass amino acids as determined by Kabat plus Chothia

TABLE 24
Antibody 29E-CDR Sequences

| | | Exem- | | Cho- | | Con- | |
|---|---|---|---|---|---|---|---|
| | | plary* | Kabat | thia | AbM | tact | IMGT |
| VH | VH | GFTFRS | SYGMH | GFTFRS | GFTFRS | RSYGMH | GFTF |
| CDR | CDR1 | YGMH | (SEQ | Y | YGMH | (SEQ | RSYG |
| Seq. | | (SEQ | ID | (SEQ | (SEQ | ID | (SEQ |
| | | ID | NO: | ID | ID | NO: | ID |
| | | NO: | 349) | NO: | NO: | 367) | NO: |
| | | 343) | | 355) | 361) | | 373) |
| | VH | VITYDG | VITYDG | YDGI | VITYDG | WVAVIT | ITYD |
| | CDR2 | INKYYA | INKYYA | (SEQ | INKY | YDGINK | GINK |
| | | DSVEG | DSVEG | ID | (SEQ | Y | (SEQ |
| | | (SEQ | (SEQ | NO: | ID | (SEQ | ID |
| | | ID | ID | 356) | NO: | ID | NO: |
| | | NO: | NO: | | 362) | NO: | 374) |
| | | 344) | 350) | | | 368) | |
| | VH | DGVYYG | DGVYYG | GVYYGV | DGVYYG | ARDGVY | ARDG |
| | CDR3 | VYDY | VYDY | YD | VYDY | YGVYD | VYYG |
| | | (SEQ | (SEQ | (SEQ | (SEQ | (SEQ | VYDY |
| | | ID | ID | ID | ID | ID | (SEQ |
| | | NO: | NO: | NO: | NO: | NO: | ID |
| | | 345) | 351) | 357) | 363) | 369) | NO: |
| | | | | | | | 375) |
| VL | VL | KSSQSV | KSSQSV | SQSVLF | KSSQSV | LFSSNN | QSVL |
| CDR | CDR1 | LFSSNN | LFSSNN | SSNNKN | LFSSNN | KNYLA | FSSN |
| Seq. | | KNYLA | KNYLA | Y | KNYLA | WY | NKNY |
| | | (SEQ | (SEQ | (SEQ | (SEQ | (SEQ | (SEQ |
| | | ID | ID | ID | ID | ID | ID |
| | | NO: | NO: | NO: | NO: | NO: | NO: |
| | | 346) | 352) | 358) | 364) | 370) | 376) |
| | VL | WASTRE | WASTRE | WAS | WASTRE | LLIYWA | WAS |
| | CDR2 | S | S | (SEQ | S | STRE | (SEQ |
| | | (SEQ | (SEQ | ID | (SEQ | (SEQ | ID |
| | | ID | ID | NO: | ID | ID | NO: |
| | | NO: | NO: | 359) | NO: | NO: | 377) |
| | | 347) | 353) | | 365) | 371) | |
| | VL | QQFHSY | QQFHSY | FHSYPL | QQFHSY | QQFHSY | QQFH |
| | CDR3 | PLT | PLT | (SEQ | PLT | PL | SYPL |
| | | (SEQ | (SEQ | ID | (SEQ | (SEQ | T |
| | | ID | ID | NO: | ID | ID | (SEQ |
| | | NO: | NO: | 360) | NO: | NO: | ID |
| | | 348) | 354) | | 366) | 372) | NO: |
| | | | | | | | 378) |

VH Sequence*:
QVQLVESGGGVVQPGRSLRLSCAASGFTFRSYGMHWVRQAPGKGLEWVAVITYDGINKYYADSVEGRFTISRDNSKNTLYLQMNSLRAEDTAVYYCARDGVYYGVYDYWGQGTLVTVSS (SEQ ID NO: 379)
VL Sequence*:
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQFHSYPLTFGGGTKVEIK (SEQ ID NO: 380)
*Exemplary CDR sequences encompass amino acids as determined by Kabat plus Chothia

TABLE 25
Antibody 39A-CDR Sequences

| | | Exem- | | Cho- | | Con- | |
|---|---|---|---|---|---|---|---|
| | | plary* | Kabat | thia | AbM | tact | IMGT |
| VH | VH | GGTFSS | SNAIG | GGTFSS | GGTFSS | SSNAIG | GGTF |
| CDR | CDR1 | NAIG | (SEQ | N | NAIG | (SEQ | SSNA |
| Seq. | | (SEQ | ID | (SEQ | (SEQ | ID | (SEQ |
| | | ID | NO: | ID | ID | NO: | ID |
| | | NO: | 387) | NO: | NO: | 405) | NO: |
| | | 381) | | 393) | 399) | | 411) |
| | VH | SIIPII | SIIPII | PIIG | SIIPII | WMGSII | IIPI |
| | CDR2 | GFANYA | GFANYA | (SEQ | GFAN | PIIGFA | IGFA |
| | | QKFQG | QKFQG | ID | (SEQ | N | (SEQ |
| | | (SEQ | (SEQ | NO: | ID | (SEQ | ID |
| | | ID | ID | 394) | NO: | ID | NO: |
| | | NO: | NO: | | 400) | NO: | 412) |
| | | 382) | 388) | | | 406) | |
| | VH | DSGYYY | DSGYYY | SGYYYG | DSGYYY | ARDSGY | ARDS |
| | CDR3 | GASSFG | GASSFG | ASSFG | GASSFG | YYGASS | GYYY |
| | | MDV | MDV | MD | MDV | FGMD | GASS |
| | | (SEQ | (SEQ | (SEQ | (SEQ | (SEQ | FGMD |
| | | ID | ID | ID | ID | ID | V |
| | | NO: | NO: | NO: | NO: | NO: | (SEQ |
| | | 383) | 389) | 395) | 401) | 407) | ID |
| | | | | | | | NO: |
| | | | | | | | 413) |
| VL | VL | RASQSV | RASQSV | SQSVSS | RASQSV | SSNLAW | QSVS |
| CDR | CDR1 | SSNLA | SSNLA | N | SSNLA | Y | SN |
| Seq. | | (SEQ | (SEQ | (SEQ | (SEQ | (SEQ | (SEQ |
| | | ID | ID | ID | ID | ID | ID |
| | | NO: | NO: | NO: | NO: | NO: | NO: |
| | | 384) | 390) | 396) | 402) | 408) | 414) |
| | VL | GASTRA | GASTRA | GAS | GASTRA | LLIYGA | GAS |
| | CDR2 | T | T | (SEQ | T | STRA | (SEQ |
| | | (SEQ | (SEQ | ID | (SEQ | (SEQ | ID |
| | | ID | ID | NO: | ID | ID | NO: |
| | | NO: | NO: | 397) | NO: | NO: | 415) |
| | | 385) | 391) | | 403) | 409) | |
| | VL | EQYNNL | EQYNNL | YNNLPL | EQYNNL | EQYNNL | EQYNNL |
| | CDR3 | PLT | PLT | (SEQ | PLT | PL | PLT |
| | | (SEQ | (SEQ | ID | (SEQ | (SEQ | (SEQ |
| | | ID | ID | NO: | ID | ID | ID |
| | | NO: | NO: | 398) | NO: | NO: | NO: |
| | | 386) | 392) | | 404) | 410) | 416) |

VH Sequence*:
QVQLVQSGAEVKKPGSSVKVSCKASGGTFSSNAIGWVRQAPGQGLEWMGSIIPIIGFANYAQKFQGRVTITADESTSTAYMELSSLRSEDTAVYYCARDSGYYYGASSFGMDVWGQGTTVTVSS (SEQ ID NO: 417)
VL Sequence*:
EIVMTQSPATLSVSPGERATLSCRASQSVSSNLAWYQQKPGQAPRLLIYGASTRATGIPARFSGSGSGTEFTLTISSLQSEDFAVYYCEQYNNLPLTFGGGTKVEIK (SEQ ID NO: 418)
*Exemplary CDR sequences encompass amino acids as determined by Kabat plus Chothia

TABLE 26
Antibody 43B-CDR Sequences

| | | Exem- | | Cho- | | Con- | |
|---|---|---|---|---|---|---|---|
| | | plary* | Kabat | thia | AbM | tact | IMGT |
| VH | VH | GGSISS | SGQYWS | GGSISS | GGSISS | SSGQYW | GGSI |
| CDR | CDR1 | GQYWS | (SEQ | GQ | GQYWS | S | SSGQ |
| Seq. | | (SEQ | ID | (SEQ | (SEQ | (SEQ | Y |
| | | ID | NO: | ID | ID | ID | (SEQ |
| | | NO: | 425) | NO: | NO: | NO: | ID |
| | | 419) | | 431) | 437) | 443) | NO: |
| | | | | | | | 449) |
| | VH | EIYYSG | EIYYSG | YSG | EIYYSG | WIGEIY | IYYS |
| | CDR2 | STRYNP | STRYNP | (SEQ | STR | YSGSTR | GST |
| | | SLKS | SLKS | ID | (SEQ | (SEQ | (SEQ |
| | | (SEQ | (SEQ | NO: | ID | ID | ID |
| | | ID | ID | 432) | NO: | NO: | NO: |
| | | NO: | NO: | | 438) | 444) | 450) |
| | | 420) | 426) | | | | |
| | VH | DAPYYY | DAPYYY | APYYYG | DAPYYY | ARDAPY | ARDA |
| | CDR3 | GGGYY | GGGYY | GGYYY | GGGYY | YYGGG | PYYY |
| | | YYMDV | YYMDV | YMD | YYMDV | YYYYMD | GGGY |
| | | (SEQ | (SEQ | (SEQ | (SEQ | (SEQ | YYYM |
| | | ID | ID | ID | ID | ID | DV |
| | | NO: | NO: | NO: | NO: | NO: | (SEQ |
| | | 421) | 427) | 433) | 439) | 445) | ID |
| | | | | | | | NO: |
| | | | | | | | 451) |
| VL | VL | RASQSV | RASQSV | SQSVSS | RASQSV | SSSYLA | QSVS |
| CDR | CDR1 | SSSYLA | SSSYLA | SY | SSSYLA | WY | SSY |
| Seq. | | (SEQ | (SEQ | (SEQ | (SEQ | (SEQ | (SEQ |
| | | ID | ID | ID | ID | ID | ID |
| | | NO: | NO: | NO: | NO: | NO: | NO: |
| | | 422) | 428) | 434) | 440) | 446) | 452) |
| | VL | GASSRA | GASSRA | GAS | GASSRA | LLIYGA | GAS |
| | CDR2 | T | T | (SEQ | T | SSRA | (SEQ |
| | | (SEQ | (SEQ | ID | (SEQ | (SEQ | ID |
| | | ID | ID | NO: | ID | ID | NO: |
| | | NO: | NO: | 435) | NO: | NO: | 453) |
| | | 423) | 429) | | 441) | 447) | |
| | VL | QQVGVV | QQVGVV | VGVVPY | QQVGVV | QQVGVV | QQVGVV |
| | CDR3 | PYT | PYT | (SEQ | PYT | PY | PYT |
| | | (SEQ | (SEQ | ID | (SEQ | (SEQ | (SEQ |
| | | ID | ID | NO: | ID | ID | ID |
| | | NO: | NO: | 436) | NO: | NO: | NO: |
| | | 424) | 430) | | 442) | 448) | 454) |

VH Sequence*:
QVQLQESGPGLVKPSQTLSLTCTVSGGSISSGQYWSWIRQHPGKGLEWIGEIYYSGSTRYNPSLKSRVTISVDTSKNQFSLKLSSVTAADTAVYYCARDAPYYYGGGYYYYMDVWGKGTTVTVSS (SEQ ID NO: 455)
VL Sequence*:
EIVLTQSPGTLSLSPGERATLSCRASQSVSSSYLAWYQQKPGQAPRLLIYGASSRATGIPDRFSGSGSGTDFTLTISRLEPEDFAVYYCQQVGVVPYTFGGGTKVEIK (SEQ ID NO: 456)
*Exemplary CDR sequences encompass amino acids as determined by Kabat & Chothia

TABLE 27
Antibody 43B1-CDR Sequences

| | | Exem- | | Cho- | | Con- | |
|---|---|---|---|---|---|---|---|
| | | plary* | Kabat | thia | AbM | tact | IMGT |
| VH | VH | GGSISS | SGQYWS | GGSISS | GGSISS | SSGQYW | GGSI |
| CDR | CDR1 | GQYWS | (SEQ | GQ | GQYWS | S | SSGQ |
| Seq. | | (SEQ | ID | (SEQ | (SEQ | (SEQ | Y |
| | | ID | NO: | ID | ID | ID | (SEQ |
| | | NO: | 463) | NO: | NO: | NO: | ID |
| | | 457) | | 469) | 475) | 481) | NO: |
| | | | | | | | 487) |
| | VH | EIYYSG | EIYYSG | YSG | EIYYSG | WIGEIY | IYYS |
| | CDR2 | STRYNP | STRYNP | (SEQ | STR | YSGSTR | GST |
| | | SLKS | SLKS | ID | (SEQ | (SEQ | (SEQ |
| | | (SEQ | (SEQ | NO: | ID | ID | ID |
| | | ID | ID | 470) | NO: | NO: | NO: |
| | | NO: | NO: | | 476) | 482) | 488) |
| | | 458) | 464) | | | | |
| | VH | DAPYYY | DAPYYY | APYYYG | DAPYYY | ARDAPY | ARDA |
| | CDR3 | GGGYY | GGGYY | GGYYY | GGGYY | YYGGG | PYYY |
| | | YYMDV | YYMDV | YMD | YYMDV | YYYYMD | GGGY |
| | | (SEQ | (SEQ | (SEQ | (SEQ | (SEQ | YYYM |
| | | ID | ID | ID | ID | ID | DV |
| | | NO: | NO: | NO: | NO: | NO: | (SEQ |
| | | 459) | 465) | 471) | 477) | 483) | ID |
| | | | | | | | NO: |
| | | | | | | | 489) |
| VL | VL | RASESV | RASESV | SESVDS | RASESV | DSSYLA | ESVD |
| CDR | CDR1 | DSSYLA | DSSYLA | SY | DSSYLA | WY | SSY |
| Seq. | | (SEQ | (SEQ | (SEQ | (SEQ | (SEQ | (SEQ |
| | | ID | ID | ID | ID | ID | ID |
| | | NO: | NO: | NO: | NO: | NO: | NO: |
| | | 460) | 466) | 472) | 478) | 484) | 490) |
| | VL | GASTRQ | GASTRQ | GAS | GASTRQ | LLIYGA | GAS |
| | CDR2 | T | T | (SEQ | T | STRQ | (SEQ |
| | | (SEQ | (SEQ | ID | (SEQ | (SEQ | ID |
| | | ID | ID | NO: | ID | ID | NO: |
| | | NO: | NO: | 473) | NO: | NO: | 491) |
| | | 461) | 467) | | 479) | 485) | |
| | VL | QQAGVV | QQAGVV | AGVVPY | QQAGVV | QQAGVV | QQAG |
| | CDR3 | PYT | PYT | (SEQ | PYT | PY | VVPY |
| | | (SEQ | (SEQ | ID | (SEQ | (SEQ | T |
| | | ID | ID | NO: | ID | ID | (SEQ |
| | | NO: | NO: | 474) | NO: | NO: | ID |
| | | 462) | 468) | | 480) | 486) | NO: |
| | | | | | | | 492) |

VH Sequence*:
QVQLQESGPGLVKPSQTLSLTCTVSGGSISSGQYWSWIRQHPGKGLEWIGEIYYSGSTRYNPSLKSRVTISVDTSKNQFSLKLSSVTAADTAVYYCARDAPYYYGGGYYYYMDVWGKGTTVTVSS (SEQ ID NO: 493)
VL Sequence*:
EIVLTQSPGTLSLSPGERATLSCRASESVDSSYLAWYQQKPGQAPRLLIYGASTRQTGIPDRFSGSGSGTDFTLTISRLEPEDFAVYYCQQAGVVPYTFGGGTKVEIK (SEQ ID NO: 494)
*Exemplary CDR sequences encompass amino acids as determined by Kabat & Chothia

TABLE 28
Antibody 43B7-CDR Sequences

| | | Exem- | | Cho- | | Con- | |
|---|---|---|---|---|---|---|---|
| | | plary* | Kabat | thia | AbM | tact | IMGT |
| VH | VH | GGSISS | SGQYWS | GGSISS | GGSISS | SSGQYW | GGSI |
| CDR | CDR1 | GQYWS | (SEQ | GQ | GQYWS | S | SSGQ |
| Seq. | | (SEQ | ID | (SEQ | (SEQ | (SEQ | Y |
| | | ID | NO: | ID | ID | ID | (SEQ |
| | | NO: | 501) | NO: | NO: | NO: | ID |
| | | 495) | | 507) | 513) | 519) | NO: |
| | | | | | | | 525) |
| | VH | EIYYSG | EIYYSG | YSG | EIYYSG | WIGEIY | IYYS |
| | CDR2 | STRYNP | STRYNP | (SEQ | STR | YSGSTR | GST |
| | | SLKS | SLKS | ID | (SEQ | (SEQ | (SEQ |
| | | (SEQ | (SEQ | NO: | ID | ID | ID |
| | | ID | ID | 508) | NO: | NO: | NO: |
| | | NO: | NO: | | 514) | 520) | 526) |
| | | 496) | 502) | | | | |
| | VH | DAPYYY | DAPYYY | APYYYG | DAPYYY | ARDAPY | ARDA |
| | CDR3 | GGGYY | GGGYY | GGYYY | GGGYY | YYGGG | PYYY |
| | | YYMDV | YYMDV | YMD | YYMDV | YYYYMD | GGGY |
| | | (SEQ | (SEQ | (SEQ | (SEQ | (SEQ | YYYM |
| | | ID | ID | ID | ID | ID | DV |
| | | NO: | NO: | NO: | NO: | NO: | (SEQ |
| | | 497) | 503) | 509) | 515) | 521) | ID |
| | | | | | | | NO: |
| | | | | | | | 527) |
| VL | VL | RASESV | RASESV | SESVDS | RASESV | DSSYLA | ESVD |
| CDR | CDR1 | DSSYLA | DSSYLA | SY | DSSYLA | WY | SSY |
| Seq. | | (SEQ | (SEQ | (SEQ | (SEQ | (SEQ | (SEQ |
| | | ID | ID | ID | ID | ID | ID |
| | | NO: | NO: | NO: | NO: | NO: | NO: |
| | | 498) | 504) | 510) | 516) | 522) | 528) |
| | VL | GADSRA | GADSRA | GAD | GADSRA | LLIYGA | GAD |
| | CDR2 | T | T | (SEQ | T | DSRA | (SEQ |
| | | (SEQ | (SEQ | ID | (SEQ | (SEQ | ID |
| | | ID | ID | NO: | ID | ID | NO: |
| | | NO: | NO: | 511) | NO: | NO: | 529) |
| | | 499) | 505) | | 517) | 523) | |
| | VL | QQDGVV | QQDGVV | DGVVPY | QQDGVV | QQDGVV | QQDG |
| | CDR3 | PYT | PYT | (SEQ | PYT | PY | VVPY |
| | | (SEQ | (SEQ | ID | (SEQ | (SEQ | T |
| | | ID | ID | NO: | ID | ID | (SEQ |
| | | NO: | NO: | 512) | NO: | NO: | ID |
| | | 500) | 506) | | 518) | 524) | NO: |
| | | | | | | | 530) |

VH Sequence*:
QVQLQESGPGLVKPSQTLSLTCTVSGGSISSGQYWSWIRQHPGKGLEWIGEIYYSGSTRYNPSLKSRVTISVDTSKNQFSLKLSSVTAADTAVYYCARDAPYYYGGGYYYYMDVWGKGTTVTVSS (SEQ ID NO: 531)
VL Sequence*:
EIVLTQSPGTLSLSPGERATLSCRASESVDSSYLAWYQQKPGQAPRLLIYGADSRATGIPDRFSGSGSGTDFTLTISRLEPEDFAVYYCQQDGVVPYTFGGGTKVEIK (SEQ ID NO: 532)
*Exemplary CDR sequences encompass amino acids as determined by Kabat & Chothia

TABLE 29
Antibody 43D-CDR Sequences

| | | Exemplary* | Kabat | Chothia | AbM | Contact | IMGT |
|---|---|---|---|---|---|---|---|
| VH | VH | GGSLSGYYWS | GYYWS | GGSLSGY | GGSLSGYYWS | SGYYWS | GGSLSGYY |
| CDR | CDR1 | (SEQ ID NO: 533) | (SEQ ID NO: 539) | SEQ ID NO: 545) | (SEQ ID NO: 551) | (SEQ ID NO: 557) | (SEQ ID NO: 563) |
| Seq. | VH | EIGASGSTRYNPS | EIGASGSTRYNPS | ASG | EIGASGSTR | WIGEIGASGSTR | IGASGST |
| | CDR2 | LKS | LKS | (SEQ ID NO: 546) | (SEQ ID NO: 552) | (SEQ ID NO: 558) | (SEQ ID NO: 564) |
| | | (SEQ ID NO: 534) | (SEQ ID NO: 540) | | | | |
| | VH | DTPYYYEGGYY | DTPYYYEGGYY | TPYYYEGGYYY | DTPYYYEGGYY | ARDTPYYYEGG | ARDTPYYYEGG |
| | CDR3 | YYMDV | YYMDV | YMD | YYMDV | YYYYMD | YYYYMDV |
| | | (SEQ ID NO: 535) | (SEQ ID NO: 541) | (SEQ ID NO: 547) | (SEQ ID NO: 553) | (SEQ ID NO: 559) | (SEQ ID NO: 565) |
| VL | VL | RASQSVSSSYL | RASQSVSSSYLA | SQSVSSSY | RASQSVSSSYLA | SSSYLAWY | QSVSSSY |
| CDR | CDR1 | A | (SEQ ID NO: 542) | (SEQ ID NO: 548) | (SEQ ID NO: 554) | (SEQ ID NO: 560) | (SEQ ID NO: 566) |
| Seq. | | (SEQ ID NO: 536) | | | | | |
| | VL | GASSRAT | GASSRAT | GAS | GASSRAT | LLIYGASSRA | GAS |
| | CDR2 | (SEQ ID NO: 537) | (SEQ ID NO: 543) | (SEQ ID NO: 549) | (SEQ ID NO: 555) | (SEQ ID NO: 561 | (SEQ ID NO: 567) |
| | VL | QQVGVVPYT | QQVGVVPYT | VGVVPY | QQVGVVPYT | QQVGVVPY | QQVGVVPYT |
| | CDR3 | (SEQ ID NO: 538) | (SEQ ID NO: 544) | (SEQ ID NO: 550) | (SEQ ID NO: 556) | (SEQ ID NO: 562) | (SEQ ID NO: 568) |

VH Sequence*:
QVQLQQWGAGLLKPSETLSLTCAVYGGSLSGYYWSWIRQPPGKGLEWIGEIGASGSTRYNPSLKSRVTISVDTSKNQFSLKLSSVTAADTAVYY
CARDTPYYYEGGYYYYMDVWGKGTTVTVSS (SEQ ID NO: 569)
VL Sequence*:
EIVLTQSPGTLSLSPGERATLSCRASQSVSSSYLAWYQQKPGQAPRLLIYGASSRATGIPDRFSGSGSGTDFTLTISRLEPEDFAVYYCQQVGVVPY
TFGGGTKVEIK (SEQ ID NO: 570)
*Exemplary CDR sequences encompass amino acids as determined by Kabat & Chothia

TABLE 30
Antibody 43D7-CDR Sequences

| | | Exemplary* | Kabat | Chothia | AbM | Contact | IMGT |
|---|---|---|---|---|---|---|---|
| VH | VH | GGSLSGYYWS | GYYWS | GGSLSGY | GGSLSGYYWS | SGYYWS | GGSLSGYY |
| CDR | CDR1 | (SEQ ID NO: 571) | (SEQ ID NO: 577) | (SEQ ID NO: 583) | (SEQ ID NO: 589) | (SEQ ID NO: 595) | (SEQ ID NO: 601) |
| Seq. | VH | EIGASGSTRYNPS | EIGASGSTRYNPS | ASG | EIGASGSTR | WIGEIGASGSTR | IGASGST |
| | CDR2 | LKS | LKS | (SEQ ID NO: 584) | (SEQ ID NO: 590) | (SEQ ID NO: 596) | (SEQ ID NO: 602) |
| | | (SEQ ID NO: 572) | (SEQ ID NO: 578) | | | | |
| | VH | DTPYYYEGGYY | DTPYYYEGGYY | TPYYYEGGYYY | DTPYYYEGGYY | ARDTPYYYEGG | ARDTPYYYEGG |
| | CDR3 | YYMDV | YYMDV | YMD | YYMDV | YYYYMD | YYYYMDV |
| | | (SEQ ID NO: 573) | (SEQ ID NO: 579) | (SEQ ID NO: 585) | (SEQ ID NO: 591) | (SEQ ID NO: 597) | (SEQ ID NO: 603) |
| VL | VL | RASDSVDSSYLA | RASDSVDSSYLA | SDSVDSSY | RASDSVDSSYLA | DSSYLAWY | DSVDSSY |
| CDR | CDR1 | (SEQ ID NO: 574) | (SEQ ID NO: 580) | (SEQ ID NO: 586) | (SEQ ID NO: 592) | (SEQ ID NO: 598) | (SEQ ID NO: 604) |
| Seq. | VL | GAFSRAN | GAFSRAN | GAF | GAFSRAN | LLIYGAFSRA | GAF |
| | CDR2 | (SEQ ID NO: 575) | (SEQ ID NO: 581) | (SEQ ID NO: 587) | (SEQ ID NO: 593) | (SEQ ID NO: 599) | (SEQ ID NO: 605) |
| | VL | QQAGVVPYT | QQAGVVPYT | AGVVPY | QQAGVVPYT | QQAGVVPY | QQAGVVPYT |
| | CDR3 | (SEQ ID NO: 576) | (SEQ ID NO: 582) | (SEQ ID NO: 588) | (SEQ ID NO: 594) | (SEQ ID NO: 600) | (SEQ ID NO: 606) |

VH Sequence*:
QVQLQQWGAGLLKPSETLSLTCAVYGGSLSGYYWSWIRQPPGKGLEWIGEIGASGSTRYNPSLKSRVTISVDTSKNQFSLKLSSVTAADTAVYY
CARDTPYYYEGGYYYYMDVWGKGTTVTVSS (SEQ ID NO: 607)
VL Sequence*:
EIVLTQSPGTLSLSPGERATLSCRASDSVDSSYLAWYQQKPGQAPRLLIYGAFSRANGIPDRFSGSGSGTDFTLTISRLEPEDFAVYYCQQAGVVP
YTFGGGTKVEIK (SEQ ID NO: 608)
*Exemplary CDR sequences encompass amino acids as determined by Kabat & Chothia

TABLE 31
Antibody 43D8-CDR Sequences

| | | Exemplary* | Kabat | Chothia | AbM | Contact | IMGT |
|---|---|---|---|---|---|---|---|
| VH | VH | GGSLSGYYWS | GYYWS | GGSLSGY | GGSLSGYYWS | SGYYWS | GGSLSGYY |
| CDR | CDR1 | (SEQ ID NO: 609) | (SEQ ID NO: 615) | (SEQ ID NO: 621) | (SEQ ID NO: 627) | (SEQ ID NO: 633) | (SEQ ID NO: 639) |
| Seq. | VH | EIGASGSTRYNPS | EIGASGSTRYNPS | ASG | EIGASGSTR | WIGEIGASGSTR | IGASGST |
| | CDR2 | LKS | LKS | (SEQ ID NO: 622) | (SEQ ID NO: 628) | (SEQ ID NO: 634) | (SEQ ID NO: 640) |
| | | (SEQ ID NO: 610) | (SEQ ID NO: 616) | | | | |
| | VH | DTPYYYEGGYY | DTPYYYEGGYY | TPYYYEGGYYY | DTPYYYEGGYY | ARDTPYYYEGG | ARDTPYYYEGG |
| | CDR3 | YYMDV | YYMDV | YMD | YYMDV | YYYYMD | YYYYMDV |
| | | (SEQ ID NO: 611) | (SEQ ID NO: 617) | (SEQ ID NO: 623) | (SEQ ID NO: 629) | (SEQ ID NO: 635) | (SEQ ID NO: 641) |
| VL | VL | RASQSVSSSFLA | RASQSVSSSFLA | SQSVSSSF | RASQSVSSSFLA | SSSFLAWY | QSVSSSF |
| CDR | CDR1 | (SEQ ID NO: 612) | (SEQ ID NO: 618) | (SEQ ID NO: 624) | (SEQ ID NO: 630) | (SEQ ID NO: 636) | (SEQ ID NO: 642) |
| Seq. | VL | GAYSRAT | GAYSRAT | GAY | GAYSRAT | LLIYGAYSRA | GAY |
| | CDR2 | (SEQ ID NO: 613) | (SEQ ID NO: 619) | (SEQ ID NO: 625) | (SEQ ID NO: 631) | (SEQ ID NO: 637) | (SEQ ID NO: 643) |
| | VL | QQAGVVPYT | QQAGVVPYT | AGVVPY | QQAGVVPYT | QQAGVVPY | QQAGVVPYT |
| | CDR3 | (SEQ ID NO: 614) | (SEQ ID NO: 620) | (SEQ ID NO: 626) | (SEQ ID NO: 632) | (SEQ ID NO: 638) | (SEQ ID NO: 644) |

VH Sequence*:
QVQLQQWGAGLLKPSETLSLTCAVYGGSLSGYYWSWIRQPPGKGLEWIGEIGASGSTRYNPSLKSRVTISVDTSKNQFSLKLSSVTAADTAVYY
CARDTPYYYEGGYYYYMDVWGKGTTVTVSS (SEQ ID NO: 645)
VL Sequence*:
EIVLTQSPGTLSLSPGERATLSCRASQSVSSSFLAWYQQKPGQAPRLLIYGAYSRATGIPDRFSGSGSGTDFTLTISRLEPEDFAVYYCQQAGVVP
YTFGGGTKVEIK (SEQ ID NO: 646)
*Exemplary CDR sequences encompass amino acids as determined by Kabat & Chothia

TABLE 32
Antibody 43E-CDR Sequences

| | | Exemplary* | Kabat | Chothia | AbM | Contact | IMGT |
|---|---|---|---|---|---|---|---|
| VH | VH | GGSISSGQYWS | SGQYWS | GGSISSGQ | GGSISSGQYWS | SSGQYWS | GGSISSGQY |
| CDR | CDR1 | (SEQ ID NO: 647) | (SEQ ID NO: 653) | (SEQ ID NO: 659) | (SEQ ID NO: 665) | (SEQ ID NO: 671) | (SEQ ID NO: 677) |
| Seq. | VH | EIYYSGSTRYNPS | EIYYSGSTRYNPS | YSG | EIYYSGSTR | WIGEIYYSGSTR | IYYSGST |
| | CDR2 | LKS | LKS | (SEQ ID NO: 660) | (SEQ ID NO: 666) | (SEQ ID NO: 672) | (SEQ ID NO: 678) |
| | | (SEQ ID NO: 648) | (SEQ ID NO: 654) | | | | |
| | VH | DTPYYYDGGYY | DTPYYYDGGYY | TPYYYDGGYYY | DTPYYYDGGYY | ARDTPYYYDGG | ARDTPYYYDGG |
| | CDR3 | YYMDV | YYMDV | YMD | YYMDV | YYYYMD | YYYYMDV |
| | | (SEQ ID NO: 649) | (SEQ ID NO: 655) | (SEQ ID NO: 661) | (SEQ ID NO: 667) | (SEQ ID NO: 673) | (SEQ ID NO: 679) |
| VL | VL | RASQSVSSSYLA | RASQSVSSSYLA | SQSVSSSY | RASQSVSSSYLA | SSSYLAWY | QSVSSSY |
| CDR | CDR1 | (SEQ ID NO: 650) | (SEQ ID NO: 656) | (SEQ ID NO: 662) | (SEQ ID NO: 668) | (SEQ ID NO: 674) | (SEQ ID NO: 680) |
| Seq. | VL | GASSRAT | GASSRAT | GAS | GASSRAT | LLIYGASSRA | GAS |
| | CDR2 | (SEQ ID NO: 651) | (SEQ ID NO: 657) | (SEQ ID NO: 663) | (SEQ ID NO: 669) | (SEQ ID NO: 675) | (SEQ ID NO: 681) |
| | VL | QQVGVVPYT | QQVGVVPYT | VGVVPY | QQVGVVPYT | QQVGVVPY | QQVGVVPYT |
| | CDR3 | (SEQ ID NO: 652) | (SEQ ID NO: 658) | (SEQ ID NO: 664) | (SEQ ID NO: 670) | (SEQ ID NO: 676) | (SEQ ID NO: 682) |

VH Sequence*:
QVQLQESGPGLVKPSQTLSLTCTVSGGSISSGQYWSWIRQHPGKGLEWIGEIYYSGSTRYNPSLKSRVTISVDTSKDQFSLKLSSVTAADTAVYY
CARDTPYYYDGGYYYYMDVWGKGTTVTVSS (SEQ ID NO: 683)
VL Sequence*:
EIVLTQSPGTLSLSPGERATLSCRASQSVSSSYLAWYQQKPGQAPRLLIYGASSRATGIPDRFSGSGSGTDFTLTISRLEPEDFAVYYCQQVGVVPY
TFGGGTKVEIK (SEQ ID NO: 684)
*Exemplary CDR sequences encompass amino acids as determined by Kabat & Chothia

TABLE 33
Antibody 43Ea-CDR Sequences

| | | Exemplary* | Kabat | Chothia | AbM | Contact | IMGT |
|---|---|---|---|---|---|---|---|
| VH | VH | GGSISSGQYWS | SGQYWS | GGSISSGQ | GGSISSGQYWS | SSGQYWS | GGSISSGQY |
| CDR | CDR1 | (SEQ ID NO: 685) | (SEQ ID NO: 691) | (SEQ ID NO: 697) | (SEQ ID NO: 703) | (SEQ ID NO: 709) | (SEQ ID NO: 715) |
| Seq. | VH | EIYYSGSTRYNPS | EIYYSGSTRYNPS | YSG | EIYYSGSTR | WIGEIYYSGSTR | IYYSGST |
| | CDR2 | LKS | LKS | (SEQ ID NO: 698) | (SEQ ID NO: 704) | (SEQ ID NO: 710) | (SEQ ID NO: 716) |
| | | (SEQ ID NO: 686) | (SEQ ID NO: 692) | | | | |
| | VH | DTPYYYDGGYY | DTPYYYDGGYY | TPYYYDGGYYY | DTPYYYDGGYY | ARDTPYYYDGG | ARDTPYYYDGG |
| | CDR3 | YYMDV | YYMDV | YMD | YYMDV | YYYYMD | YYYYMDV |
| | | (SEQ ID NO: 687) | (SEQ ID NO: 693) | (SEQ ID NO: 699) | (SEQ ID NO: 705) | (SEQ ID NO: 711) | (SEQ ID NO: 717) |
| VL | VL | RASQSVSSSYLA | RASQSVSSSYLA | SQSVSSSY | RASQSVSSSYLA | SSSYLAWY | QSVSSSY |
| CDR | CDR1 | (SEQ ID NO: 688) | (SEQ ID NO: 694) | (SEQ ID NO: 700) | (SEQ ID NO: 706) | (SEQ ID NO: 712) | (SEQ ID NO: 718) |
| Seq. | VL | GASSRAT | GASSRAT | GAS | GASSRAT | LLIYGASSRA | GAS |
| | CDR2 | (SEQ ID NO: 689) | (SEQ ID NO: 695) | (SEQ ID NO: 701) | (SEQ ID NO: 707) | (SEQ ID NO: 713) | (SEQ ID NO: 719) |
| | VL | QQVGVVPYT | QQVGVVPYT | VGVVPY | QQVGVVPYT | QQVGVVPY | QQVGVVPYT |
| | CDR3 | (SEQ ID NO: 690) | (SEQ ID NO: 696) | (SEQ ID NO: 702) | (SEQ ID NO: 708) | (SEQ ID NO: 714) | (SEQ ID NO: 720) |

VH Sequence*:
QVQLQESGPGLVKPSQTLSLTCTVSGGSISSGQYWSWIRQHPGKGLEWIGEIYYSGSTRYNPSLKSRVTISVDTSKNQFSLKLSSVTAADTAVYY
CARDTPYYYDGGYYYYMDVWGKGTTVTVSS (SEQ ID NO: 721)
VL Sequence*:
EIVLTQSPGTLSLSPGERATLSCRASQSVSSSYLAWYQQKPGQAPRLLIYGASSRATGIPDRFSGSGSGTDFTLTISRLEPEDFAVYYCQQVGVVPY
TFGGGTKVEIK (SEQ ID NO: 722)
*Exemplary CDR sequences encompass amino acids as determined by Kabat & Chothia

TABLE 34
Antibody 54E-CDR Sequences

| | | Exemplary* | Kabat | Chothia | AbM | Contact | IMGT |
|---|---|---|---|---|---|---|---|
| VH | VH | GYTFANYYMH | NYYMH | GYTFANY | GYTFANYYMH | ANYYMH | GYTFANYY |
| CDR | CDR1 | (SEQ ID NO: 723) | (SEQ ID NO: 729) | (SEQ ID NO: 735) | (SEQ ID NO: 741) | (SEQ ID NO: 747) | (SEQ ID NO: 753) |
| Seq. | VH | IINPSGGITVYAQ | IINPSGGITVYAQ | PSGG | IINPSGGITV | WMGIINPSGGIT | INPSGGIT |
| | CDR2 | KFQG | KFQG | (SEQ ID NO: 736) | (SEQ ID NO: 742) | V | (SEQ ID NO: 754) |
| | | (SEQ ID NO: 724) | (SEQ ID NO: 730) | | | (SEQ ID NO: 748) | |
| | VH | GGSKVAALAFDI | GGSKVAALAFDI | GSKVAALAFD | GGSKVAALAFDI | ARGGSKVAALA | ARGGSKVAALA |
| | CDR3 | (SEQ ID NO: 725) | (SEQ ID NO: 731) | (SEQ ID NO: 737) | (SEQ ID NO: 743) | FD | FDI |
| | | | | | | (SEQ ID NO: 749) | (SEQ ID NO: 755) |
| VL | VL | QASQDISNSLN | QASQDISNSLN | SQDISNS | QASQDISNSLN | SNSLNWY | QDISNS |
| CDR | CDR1 | (SEQ ID NO: 726) | (SEQ ID NO: 732) | (SEQ ID NO: 738) | (SEQ ID NO: 744) | (SEQ ID NO: 750) | (SEQ ID NO: 756) |
| Seq. | VL | DASNLET | DASNLET | DAS | DASNLET | LLIYDASNLE | DAS |
| | CDR2 | (SEQ ID NO: 727) | (SEQ ID NO: 733) | (SEQ ID NO: 739) | (SEQ ID NO: 745) | (SEQ ID NO: 751) | (SEQ ID NO: 757) |
| | VL | QQYNFHPLT | QQYNFHPLT | YNFHPL | QQYNFHPLT | QQYNFHPL | QQYNFHPLT |
| | CDR3 | (SEQ ID NO: 728) | (SEQ ID NO: 734) | (SEQ ID NO: 740) | (SEQ ID NO: 746) | (SEQ ID NO: 752) | (SEQ ID NO: 758) |

VH Sequence*:
QVQLVQSGAEVKKPGASVKVSCKASGYTFANYYMHWVRQAPGQGLEWMGIINPSGGITVYAQKFQGRVTMTRDTSTSTVYMELSSLRSEDT
AVYYCARGGSKVAALAFDIWGQGTMVTVSS (SEQ ID NO: 759)
VL Sequence*:
DIQMTQSPSSLSASVGDRVTITCQASQDISNSLNWYQQKPGKAPKLLIYDASNLETGVPSRFSGSRSGTDFTFTISSLQPEDIATYYCQQYNFHPLT
FGGGTKVEIK (SEQ ID NO: 760)
*Exemplary CDR sequences encompass amino acids as determined by Kabat plus Chothia

TABLE 35
Consensus CDRs

| Antibody Group | | 1 | 25 | 29 | 39 | 43 | 54 |
|---|---|---|---|---|---|---|---|
| VH | VH | GFTFSx[D/S]YAM | GYTFx[D/R]x[S/V] | GFTFx[H/R]Sx[R/Y] | GGTFSSNAIG | GGSx[I/L]SSGx[Q/Y] | GYTFANYYMH |
| CDR | CDR1 | x[A/G] | YGIS | GMH | (SEQ ID NO: 791) | YWS | (SEQ ID NO: 803) |
| Seq.* | | (SEQ ID NO: 773) | (SEQ ID NO: 779) | (SEQ ID NO: 785) | | (SEQ ID NO: 797) | |
| | VH | x[A/T]ISGSGGLT | Wx[I/V]APYx[S/N] | VITYDGINKYYA | SIIPIIGFANYAQK | EIx[Y/G]x[Y/A]SG | IINPSGGITVYAQ |
| | CDR2 | YYADSVKG (SEQ | GNTNYAQKLQG | DSVEG | FQG | STRYNPSLKS | KFQG |
| | | ID NO: 774) | (SEQ ID NO: 780) | (SEQ ID NO: 786) | (SEQ ID NO: 792) | (SEQ ID NO: 798) | (SEQ ID NO: 804) |
| | VH | APYGYYMDV | DAGTYSPx[F/Y]G | DGVYYGVYDY | DSGYYYGASSFG | Dx[T/A]PYYYx[E/G/D] | GGSKVAALAFDI |
| | CDR3 | (SEQ ID NO: 775) | YGMDV | (SEQ ID NO: 787) | MDV | GGYYYYMD | (SEQ ID NO: 805) |
| | | | (SEQ ID NO: 781) | | (SEQ ID NO: 793) | V | |
| | | | | | | (SEQ ID NO: 799) | |
| VL | VL | RASQSISSWLA | x[R/Q]ASx[Q/E/H] | KSSQSVLFSSNN | RASQSVSSNLA | RASx[Q/E/D]SVx[ | QASQDISNSLN |
| CDR | CDR1 | (SEQ ID NO: 776) | SIx[S/D/N]x[S/N] | KNYLA | (SEQ ID NO: 794) | S/D]SSx[Y/F]LA | (SEQ ID NO: 806) |
| Seq.* | | | WLA | (SEQ ID NO: 788) | | (SEQ ID NO: 800) | |
| | | | (SEQ ID NO: 782) | | | | |
| | VL | KASSLES | x[K/S]Ax[S/Y]x[S/Y/N] | WASTRES | GASTRAT | GAx[S/D/F/Y]x[S/T] | DASNLET |
| | CDR2 | (SEQ ID NO: 777) | LEx[S/Y] | (SEQ ID NO: 789) | (SEQ ID NO: 795) | Rx[A/Q]x[T/N] | (SEQ ID NO: 807) |
| | | | (SEQ ID NO: 783) | | | (SEQ ID NO: 801) | |
| | VL | QQYKSYIT | Qx[Q/L/R]FQx[S/K] | QQFHSYPLT | EQYNNLPLT | QQx[V/A/D]GVVP | QQYNFHPLT |
| | CDR3 | (SEQ ID NO: 778) | LPPFT | (SEQ ID NO: 790) | (SEQ ID NO: 796) | YT | (SEQ ID NO: 808) |
| | | | (SEQ ID NO: 784) | | | (SEQ ID NO: 802) | |

*Exemplary CDR sequences encompass amino acids as determined by Kabat plus Chothia

TABLE 36
Human, Cynomolgus Monkey, and Mouse TF Sequences

| | Human | Cynomolgus Monkey | Mouse |
|---|---|---|---|
| Species | (Homo sapiens) | (Macaca fascicularis) | (Mus musculus) |
| Full-length | METPAWPRVPRPETA | METPAWPRVPRPETA | MAILVRPRLLAALAPT |
| sequence | VARTLLLGWVFAQV | VARTLLLGWVFAQV | FLGCLLLQVTAGAGIP |
| [signal | AGASGTTNTVAAYNL | AGASGTTNTVAAYNL | EKAFNLTWISTDFKTI |
| sequence | TWKSTNFKTILEWEP | TWKSTNFKTILEWEP | LEWQPKPTNYTYTVQ |
| underlined] | KPVNQVYTVQISTKS | KPINQVYTVQISTKSG | ISDRSRNWKNKCFSTT |
| | GDWKSKCFYTTDTEC | DWKSKCFYTADTECD | DTECDLTDEIVKDVT |
| | DLTDEIVKDVKQTYL | LTDEIVKDVKQTYLA | WAYEAKVLSVPRRNS |
| | ARVFSYPAGNVESTG | RVFSYPAGHVESTGST | VHGDGDQLVIHGEEP |
| | SAGEPLYENSPEFTPY | EEPPYENSPEFTPYLE | PFTNAPKFLPYRDTNL |
| | LETNLGQPTIQSFEQV | TNLGQPTIQSFEQVGT | GQPVIQQFEQDGRKL |
| | GTKVNVTVEDERTLV | KVNVTVQDEWTLVR | NVVVKDSLTLVRKNG |
| | RRNNTFLSLRDVFGK | RNDTFLSLRDVFGKD | TFLTLRQVFGKDLGYI |
| | DLIYTLYYWKSSSSG | LIYTLYYWKSSSSGK | ITYRKGSSTGKKTNIT |
| | KKTAKTNTNEFLIDV | KTAKTNTNEFLIDVD | NTNEFSIDVEEGVSYC |
| | DKGENYCFSVQAVIPS | KGENYCFSVQAVIPSR | FFVQAMIFSRKTNQNS |
| | RTVNRKSTDSPVECM | RTANRKSTDSPVECM | PGSSTVCTEQWKSFL |
| | GQEKGEFREIFYIIGA | GHEKGESREIFYIIGA | GETLIIVGAVVLLATIF |
| | VVFVVIILVIILAISLH | VVFVVIILVIILAISLH | IILLSISLCKRRKNRAG |
| | KCRKAGVGQSWKEN | KCKKARVGRSWKEN | QKGKNTPSRLA (SEQ |
| | SPLNVS (SEQ ID | SPLNVA (SEQ ID | ID NO: 817) |
| | NO: 809) | NO: 813) | |
| Extracellular | SGTTNTVAAYNLTWK | SGTTNTVAAYNLTWK | AGIPEKAFNLTWISTD |
| domain | STNFKTILEWEPKPVN | STNFKTILEWEPKPIN | FKTILEWQPKPTNYTY |
| (ECD) | QVYTVQISTKSGDWK | QVYTVQISTKSGDWK | TVQISDRSRNWKNKC |
| | SKCFYTTDTECDLTDE | SKCFYTADTECDLTD | FSTTDTECDLTDEIVK |
| | IVKDVKQTYLARVFS | EIVKDVKQTYLARVF | DVTWAYEAKVLSVPR |
| | YPAGNVESTGSAGEP | SYPAGHVESTGSTEEP | RNSVHGDGDQLVIHG |
| | LYENSPEFTPYLETNL | PYENSPEFTPYLETNL | EEPPFTNAPKFLPYRD |
| | GQPTIQSFEQVGTKVN | GQPTIQSFEQVGTKVN | TNLGQPVIQQFEQDG |
| | VTVEDERTLVRRNNT | VTVQDEWTLVRRND | RKLNVVVKDSLTLVR |
| | FLSLRDVFGKDLIYTL | TFLSLRDVFGKDLIYT | KNGTFLTLRQVFGKD |
| | YYWKSSSSGKKTAKT | LYYWKSSSSGKKTAK | LGYIITYRKGSSTGKK |
| | NTNEFLIDVDKGENY | TNTNEFLIDVDKGEN | TNITNTNEFSIDVEEG |
| | CFSVQAVIPSRTVNRK | YCFSVQAVIPSRRTAN | VSYCFFVQAMIFSRKT |
| | STDSPVECMGQEKGE | RKSTDSPVECMGHEK | NQNSPGSSTVCTEQW |
| | FRE (SEQ ID NO: 810) | GESRE (SEQ ID | KSFLGE (SEQ ID |
| | | NO: 814) | NO: 818) |
| Sequence of | SGTTNTVAAYNLTWK | SGTTNTVAAYNLTWK | AGIPEKAFNLTWISTD |
| TF ECD-His | STNFKTILEWEPKPVN | STNFKTILEWEPKPIN | FKTILEWQPKPTNYTY |
| (TF-His) | QVYTVQISTKSGDWK | QVYTVQISTKSGDWK | TVQISDRSRNWKNKC |
| protein | SKCFYTTDTECDLTDE | SKCFYTADTECDLTD | FSTTDTECDLTDEIVK |
| | IVKDVKQTYLARVFS | EIVKDVKQTYLARVF | DVTWAYEAKVLSVPR |
| | YPAGNVESTGSAGEP | SYPAGHVESTGSTEEP | RNSVHGDGDQLVIHG |
| | LYENSPEFTPYLETNL | PYENSPEFTPYLETNL | EEPPFTNAPKFLPYRD |
| | GQPTIQSFEQVGTKVN | GQPTIQSFEQVGTKVN | TNLGQPVIQQFEQDG |
| | VTVEDERTLVRRNNT | VTVQDEWTLVRRND | RKLNVVVKDSLTLVR |
| | FLSLRDVFGKDLIYTL | TFLSLRDVFGKDLIYT | KNGTFLTLRQVFGKD |
| | YYWKSSSSGKKTAKT | LYYWKSSSSGKKTAK | LGYIITYRKGSSTGKK |
| | NTNEFLIDVDKGENY | TNTNEFLIDVDKGEN | TNITNTNEFSIDVEEG |
| | CFSVQAVIPSRTVNRK | YCFSVQAVIPSRRTAN | VSYCFFVQAMIFSRKT |
| | STDSPVECMGQEKGE | RKSTDSPVECMGHEK | NQNSPGSSTVCTEQW |
| | FRETGHHHHHH (SEQ | GESRETGHHHHHH | KSFLGETGHHHHHH |
| | ID NO: 811) | (SEQ ID NO: 815) | (SEQ ID NO: 819) |
| Sequence of | SGTTNTVAAYNLTWK | SGTTNTVAAYNLTWK | AGIPEKAFNLTWISTD |
| TF ECD-Fc | STNFKTILEWEPKPVN | STNFKTILEWEPKPIN | FKTILEWQPKPTNYTY |
| (TF-Fc) | QVYTVQISTKSGDWK | QVYTVQISTKSGDWK | TVQISDRSRNWKNKC |
| fusion protein | SKCFYTTDTECDLTDE | SKCFYTADTECDLTD | FSTTDTECDLTDEIVK |
| | IVKDVKQTYLARVFS | EIVKDVKQTYLARVF | DVTWAYEAKVLSVPR |
| | YPAGNVESTGSAGEP | SYPAGHVESTGSTEEP | RNSVHGDGDQLVIHG |
| | LYENSPEFTPYLETNL | PYENSPEFTPYLETNL | EEPPFTNAPKFLPYRD |
| | GQPTIQSFEQVGTKVN | GQPTIQSFEQVGTKVN | TNLGQPVIQQFEQDG |
| | VTVEDERTLVRRNNT | VTVQDEWTLVRRND | RKLNVVVKDSLTLVR |
| | FLSLRDVFGKDLIYTL | TFLSLRDVFGKDLIYT | KNGTFLTLRQVFGKD |
| | YYWKSSSSGKKTAKT | LYYWKSSSSGKKTAK | LGYIITYRKGSSTGKK |
| | NTNEFLIDVDKGENY | TNTNEFLIDVDKGEN | TNITNTNEFSIDVEEG |
| | CFSVQAVIPSRTVNRK | YCFSVQAVIPSRRTAN | VSYCFFVQAMIFSRKT |
| | STDSPVECMGQEKGE | RKSTDSPVECMGHEK | NQNSPGSSTVCTEQW |
| | FRETGENLYFQGDKT | GESRETGENLYFQGD | KSFLGETGENLYFQG |
| | HTCPPCPAPELLGGPS | KTHTCPPCPAPELLGG | DKTHTCPPCPAPELLG |
| | VFLFPPKPKDTLMISR | PSVFLFPPKPKDTLMI | GPSVFLFPPKPKDTLM |
| | TPEVTCVVVDVSHED | SRTPEVTCVVVDVSH | ISRTPEVTCVVVDVSH |
| | PEVKFNWYVDGVEV | EDPEVKFNWYVDGV | EDPEVKFNWYVDGV |
| | HNAKTKPREEQYNST | EVHNAKTKPREEQYN | EVHNAKTKPREEQYN |
| | YRVVSVLTVLHQDW | STYRVVSVLTVLHQD | STYRVVSVLTVLHQD |
| | LNGKEYKCKVSNKAL | WLNGKEYKCKVSNK | WLNGKEYKCKVSNK |
| | PAPIEKTISKAKGQPR | ALPAPIEKTISKAKGQ | ALPAPIEKTISKAKGQ |
| | EPQVYTLPPSREEMTK | PREPQVYTLPPSREEM | PREPQVYTLPPSREEM |
| | NQVSLTCLVKGFYPS | TKNQVSLTCLVKGFY | TKNQVSLTCLVKGFY |
| | DIAVEWESNGQPENN | PSDIAVEWESNGQPE | PSDIAVEWESNGQPE |
| | YKTTPPVLDSDGSFFL | NNYKTTPPVLDSDGS | NNYKTTPPVLDSDGS |
| | YSKLTVDKSRWQQG | FFLYSKLTVDKSRWQ | FFLYSKLTVDKSRWQ |
| | NVFSCSVMHEALHNH | QGNVFSCSVMHEALH | QGNVFSCSVMHEALH |
| | YTQKSLSLSPGK (SEQ | NHYTQKSLSLSPGK | NHYTQKSLSLSPGK |
| | ID NO: 812) | (SEQ ID NO: 816) | (SEQ ID NO: 820) |

TABLE 39
Sequences of Anti-TF Antibodies

| Antibody | VH domain | VL domain |
|---|---|---|
| 10H10 | EVQLVQSGAEVKKPGESLRISCKGS | DIVMTQTPLSLPVTPGEPASISCKSSQ |
| (M1593) | GYTFAPYWIEWVRQMPGKGLEWM | SLLSSGNQKNYLTWYLQKPGQSPQL |
| | GDILPGTGFTTYSPSFQGHVTISADK | LIYWASTRESGVPDRFSGSGSGTDFT |
| | SISTAYLQWSSLKASDTAMYYCARS | LKISRVEAEDVGVYYCQNDYTYPLT |
| | GYYGNSGFAYWGQGTLVTVSS | FGQGTKLEIK (SEQ ID NO: 822) |
| | (SEQ ID NO: 821) | |
| TF-011 | EVQLLESGGGLVQPGGSLRLSCAAS | DIQMTQSPPSLSASAGDRVTITCRAS |
| | GFTFSNYAMSWVRQAPGKGLEWVS | QGISSRLAWYQQKPEKAPKSLIYAA |
| | SISGSGDYTYYTDSVKGRFTISRDNS | SSLQSGVPSRFSGSGSGTDFTLTISSL |
| | KNTLYLQMNSLRAEDTAVYYCARS | QPEDFATYYCQQYNSYPYTFGQGTK |
| | PWGYYLDSWGQGTLVTVSS (SEQ | LEIK (SEQ ID NO: 829) |
| | ID NO: 828) | |
| 5G9 | QVQLVESGGGVVQPGRSLRLSCKAS | DIQMTQSPSSLSASVGDRVTITCKAS |
| (humanized | GFNIKDYYMHWVRQAPGKGLEWIG | QDIRKYLNWYQQKPGKAPKLLIYY |
| TF8-5G9, | LIDPENGNTIYDPKFQGRFTISADNS | ATSLADGVPSRFSGSGSGTDYTFTIS |
| CNTO 860) | KNTLFLQMDSLRPEDTAVYYCARD | SLQPEDIATYYCLQHGESPYTFGQGT |
| | NSYYFDYWGQGTPVTVSS (SEQ ID | KLEIT (SEQ ID NO: 831) |
| | NO: 830) | |

TABLE 41
Pig TF sequences

| Species | Pig (Sus scrofa) |
|---|---|
| Full-length sequence [signal | MATPTGPPVSCPKAAVARALLLGWVLVQVAGATGTT |
| sequence underlined] | DVIVAYNLTWKSTNFKTILEWEPKPINYVYTVQISPRLG |
| | DWKNKCFHTTDTECDVTDEIMRNVKETYVARVLSYPA |
| | DTVLTAQEPPFTNSPPFTPYLDTNLGQPVIQSFEQVGTK |
| | LNVTVEAARTLVRVNGTFLRLRDVFGKDLNYTLYYW |
| | RASSTGKKKATTNTNEFLIDVDKGENYCFSVQAVIPSR |
| | RVNQKSPESRIECTSQEKAVSRELFLIVGAVVFAVIVFV |
| | LVLSVSLYKCRKERAGPSGKENAPLNVA (SEQ ID |
| | NO: 824) |
| Extracellular domain (ECD) | TGTTDVIVAYNLTWKSTNFKTILEWEPKPINYVYTVQIS |
| | PRLGDWKNKCFHTTDTECDVTDEIMRNVKETYVARVL |
| | SYPADTVLTAQEPPFTNSPPFTPYLDTNLGQPVIQSFEQ |
| | VGTKLNVTVEAARTLVRVNGTFLRLRDVFGKDLNYTL |
| | YYWRASSTGKKKATTNTNEFLIDVDKGENYCFSVQAV |
| | IPSRRVNQKSPESRIECTSQEKAVSRE (SEQ ID NO: 825) |
| Sequence of TF ECD-His (TF-His) | TGTTDVIVAYNLTWKSTNFKTILEWEPKPINYVYTVQIS |
| protein | PRLGDWKNKCFHTTDTECDVTDEIMRNVKETYVARVL |
| | SYPADTVLTAQEPPFTNSPPFTPYLDTNLGQPVIQSFEQ |
| | VGTKLNVTVEAARTLVRVNGTFLRLRDVFGKDLNYTL |
| | YYWRASSTGKKKATTNTNEFLIDVDKGENYCFSVQAV |
| | IPSRRVNQKSPESRIECTSQEKAVSRETGHHHHHH (SEQ |
| | ID NO: 826) |
| Sequence of TF ECD-Fc (TF-Fc) | TGTTDVIVAYNLTWKSTNFKTILEWEPKPINYVYTVQIS |
| fusion protein | PRLGDWKNKCFHTTDTECDVTDEIMRNVKETYVARVL |
| | SYPADTVLTAQEPPFTNSPPFTPYLDTNLGQPVIQSFEQ |
| | VGTKLNVTVEAARTLVRVNGTFLRLRDVFGKDLNYTL |
| | YYWRASSTGKKKATTNTNEFLIDVDKGENYCFSVQAV |
| | IPSRRVNQKSPESRIECTSQEKAVSRETGENLYFQGDKT |
| | HTCPPCPAPELLGGPSVFLFPPKPKDTLMISRTPEVTCV |
| | VVDVSHEDPEVKFNWYVDGVEVHNAKTKPREEQYNS |
| | TYRVVSVLTVLHQDWLNGKEYKCKVSNKALPAPIEKT |
| | ISKAKGQPREPQVYTLPPSREEMTKNQVSLTCLVKGFY |
| | PSDIAVEWESNGQPENNYKTTPPVLDSDGSFFLYSKLT |
| | VDKSRWQQGNVFSCSVMHEALHNHYTQKSLSLSPGK |
| | (SEQ ID NO: 827) |

TABLE 49
Rabbit TF sequences

| Species | Rabbit (Oryctolagus cuniculus) |
|---|---|
| Full-length sequence [signal | MAPPTRLQVPRPGTAVPYTVLLGWLLAQVARAADTTG |
| sequence underlined] | RAYNLTWKSTNFKTILEWEPKSIDHVYTVQISTRLENW |
| | KSKCFLTAETECDLTDEVVKDVGQTYMARVLSYPARN |
| | GNTTGFPEEPPFRNSPEFTPYLDTNLGQPTIQSFEQVGT |
| | KLNVTVQDARTLVRRNGTFLSLRAVFGKDLNYTLYY |
| | WRASSTGKKTATTNTNEFLIDVDKGENYCFSVQAVIPS |
| | RKRKQRSPESLTECTSREQGRAREMFFIIGAVVVVALLI |
| | IVLSVTVYKCRKARAGPSGKESSPLNIA (SEQ ID |
| | NO: 832) |
| Extracellular domain (ECD) | ADTTGRAYNLTWKSTNFKTILEWEPKSIDHVYTVQIST |
| | RLENWKSKCFLTAETECDLTDEVVKDVGQTYMARVL |
| | SYPARNGNTTGFPEEPPFRNSPEFTPYLDTNLGQPTIQSF |
| | EQVGTKLNVTVQDARTLVRRNGTFLSLRAVFGKDLNY |
| | TLYYWRASSTGKKTATTNTNEFLIDVDKGENYCFSVQ |
| | AVIPSRKRKQRSPESLTECTSREQGRAREM (SEQ ID |
| | NO: 833) |
| Sequence of TF ECD-His (TF-His) | ADTTGRAYNLTWKSTNFKTILEWEPKSIDHVYTVQIST |
| protein | RLENWKSKCFLTAETECDLTDEVVKDVGQTYMARVL |
| | SYPARNGNTTGFPEEPPFRNSPEFTPYLDTNLGQPTIQSF |
| | EQVGTKLNVTVQDARTLVRRNGTFLSLRAVFGKDLNY |
| | TLYYWRASSTGKKTATTNTNEFLIDVDKGENYCFSVQ |
| | AVIPSRKRKQRSPESLTECTSREQGRAREMTGHHHHHH |
| | (SEQ ID NO: 834) |
| Sequence of TF ECD-Fc (TF-Fc) | ADTTGRAYNLTWKSTNFKTILEWEPKSIDHVYTVQIST |
| fusion protein | RLENWKSKCFLTAETECDLTDEVVKDVGQTYMARVL |
| | SYPARNGNTTGFPEEPPFRNSPEFTPYLDTNLGQPTIQSF |
| | EQVGTKLNVTVQDARTLVRRNGTFLSLRAVFGKDLNY |
| | TLYYWRASSTGKKTATTNTNEFLIDVDKGENYCFSVQ |
| | AVIPSRKRKQRSPESLTECTSREQGRAREMENLYFQGD |
| | KTHTCPPCPAPELLGGPSVFLFPPKPKDTLMISRTPEVT |
| | CVVVDVSHEDPEVKFNWYVDGVEVHNAKTKPREEQY |
| | NSTYRVVSVLTVLHQDWLNGKEYKCKVSNKALPAPIE |
| | KTISKAKGQPREPQVYTLPPSREEMTKNQVSLTCLVKG |
| | FYPSDIAVEWESNGQPENNYKTTPPVLDSDGSFFLYSK |
| | LTVDKSRWQQGNVFSCSVMHEALHNHYTQKSLSLSPG |
| | K (SEQ ID NO: 835) |

TABLE 56
Rat TF ECD and chimeric construct ECD sequences

| Rat/Chimeric construct | Extracellular domain (ECD) sequence |
|---|---|
| rTF (rat TF) | AGTPPGKAFNLTWISTDFKTILEWQPKPTNYTYTVQISDRSRNW |
| | KYKCTGTTDTECDLTDEIVKDVNWTYEARVLSVPWRNSTHGK |
| | ETLFGTHGEEPPFTNARKFLPYRDTKIGQPVIQKYEQGGTKLKV |
| | TVKDSFTLVRKNGTFLTLRQVFGNDLGYILTYRKDSSTGRKTNT |
| | THTNEFLIDVEKGVSYCFFAQAVIFSRKTNHKSPESITKCTEQW |
| | KSVLGE (SEQ ID NO: 838) |
| h1-107_r | AGTPPGKAFNLTWISTDFKTILEWQPKPTNYTYTVQISDRSRNW |
| | KYKCTGTTDTECDLTDEIVKDVNWTYEARVLSVPWRNSTHGK |
| | ETLFGTHGEEPPFTNARKFLPYRDTKLGQPTIQSFEQVGTKVNV |
| | TVEDERTLVRRNNTFLSLRDVFGKDLIYTLYYWKSSSSGKKTA |
| | KTNTNEFLIDVDKGENYCFSVQAVIPSRTVNRKSTDSPVECMGQ |
| | EKGEFRE (SEQ ID NO: 839) |
| h1-77_r | AGTPPGKAFNLTWISTDFKTILEWQPKPTNYTYTVQISDRSRNW |
| | KYKCTGTTDTECDLTDEIVKDVNWTYEARVLSYPAGNVESTGS |
| | AGEPLYENSPEFTPYLETNLGQPTIQSFEQVGTKVNVTVEDERT |
| | LVRRNNTFLSLRDVFGKDLIYTLYYWKSSSSGKKTAKTNTNEF |
| | LIDVDKGENYCFSVQAVIPSRTVNRKSTDSPVECMGQEKGEFRE |
| | (SEQ ID NO: 840) |
| h1-38_r | AGTPPGKAFNLTWISTDFKTILEWQPKPTNYTYTVQISTKSGDW |
| | KSKCFYTTDTECDLTDEIVKDVKQTYLARVFSYPAGNVESTGS |
| | AGEPLYENSPEFTPYLETNLGQPTIQSFEQVGTKVNVTVEDERT |
| | LVRRNNTFLSLRDVFGKDLIYTLYYWKSSSSGKKTAKTNTNEF |
| | LIDVDKGENYCFSVQAVIPSRTVNRKSTDSPVECMGQEKGEFRE |
| | (SEQ ID NO: 841) |
| h39-77_r | SGTTNTVAAYNLTWKSTNFKTILEWEPKPVNQVYTVQISDRSR |
| | NWKYKCTGTTDTECDLTDEIVKDVNWTYEARVLSYPAGNVES |
| | TGSAGEPLYENSPEFTPYLETNLGQPTIQSFEQVGTKVNVTVED |
| | ERTLVRRNNTFLSLRDVFGKDLIYTLYYWKSSSSGKKTAKTNT |
| | NEFLIDVDKGENYCFSVQAVIPSRTVNRKSTDSPVECMGQEKGE |
| | FRE (SEQ ID NO: 842) |
| h78-107_r | SGTTNTVAAYNLTWKSTNFKTILEWEPKPVNQVYTVQISTKSG |
| | DWKSKCFYTTDTECDLTDEIVKDVKQTYLARVFSVPWRNSTH |
| | GTHGEEPPFTNARKFLPYRDTKLGQPTIQSFEQVGTKVNVTVED |
| | ERTLVRRNNTFLSLRDVFGKDLIYTLYYWKSSSSGKKTAKTNT |
| | NEFLIDVDKGENYCFSVQAVIPSRTVNRKSTDSPVECMGQEKGE |
| | FRE (SEQ ID NO: 843) |
| h78-107_r.v2 | SGTTNTVAAYNLTWKSTNFKTILEWEPKPVNQVYTVQISTKSG |
| | DWKSKCFYTTDTECDLTDEIVKDVKQTYLARVFSVPWRNSTH |
| | GKETLFGTHGEEPPFTNARKFLPYRDTKLGQPTIQSFEQVGTKV |
| | NVTVEDERTLVRRNNTFLSLRDVFGKDLIYTLYYWKSSSSGKK |
| | TAKTNTNEFLIDVDKGENYCFSVQAVIPSRTVNRKSTDSPVECM |
| | GQEKGEFRE (SEQ ID NO: 844) |
| h78-93_r | SGTTNTVAAYNLTWKSTNFKTILEWEPKPVNQVYTVQISTKSG |
| | DWKSKCFYTTDTECDLTDEIVKDVKQTYLARVFSVPWRNSTH |
| | GKETLFGTHGEEPPYENSPEFTPYLETNLGQPTIQSFEQVGTKVN |
| | VTVEDERTLVRRNNTFLSLRDVFGKDLIYTLYYWKSSSSGKKT |
| | AKTNTNEFLIDVDKGENYCFSVQAVIPSRTVNRKSTDSPVECMG |
| | QEKGEFRE (SEQ ID NO: 845) |
| h94-107_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLFTNARKFLPYRDTKLGQPTIQSFEQVGTKVNVTVEDE |
| | RTLVRRNNTFLSLRDVFGKDLIYTLYYWKSSSSGKKTAKTNTN |
| | EFLIDVDKGENYCFSVQAVIPSRTVNRKSTDSPVECMGQEKGEF |
| | RE (SEQ ID NO: 846) |
| h108-219_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNIGQPVIQKYEQGGTKLKVTVKDS |
| | FTLVRKNGTFLTLRQVFGNDLGYILTYRKDSSTGRKTNTTHTNE |
| | FLIDVEKGVSYCFFAQAVIFSRKTNHKSPESITKCTEQWKSVLGE |
| | (SEQ ID NO: 847) |
| h108-158_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNIGQPVIQKYEQGGTKLKVTVKDS |
| | FTLVRKNGTFLTLRQVFGNDLGYILTYRKSSSSGKKTAKTNTNE |
| | FLIDVDKGENYCFSVQAVIPSRTVNRKSTDSPVECMGQEKGEFR |
| | E (SEQ ID NO: 848) |
| h108-132_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNIGQPVIQKYEQGGTKLKVTVKDS |
| | FTLVRRNNTFLSLRDVFGKDLIYTLYYWKSSSSGKKTAKTNTN |
| | EFLIDVDKGENYCFSVQAVIPSRTVNRKSTDSPVECMGQEKGEF |
| | RE (SEQ ID NO: 849) |
| h133-158_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRKNGTFLTLRQVFGNDLGYILTYRKSSSSGKKTAKTNTNE |
| | FLIDVDKGENYCFSVQAVIPSRTVNRKSTDSPVECMGQEKGEFR |
| | E (SEQ ID NO: 850) |
| h133-145_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRKNGTFLTLRQVFGKDLIYTLYYWKSSSSGKKTAKTNTN |
| | EFLIDVDKGENYCFSVQAVIPSRTVNRKSTDSPVECMGQEKGEF |
| | RE (SEQ ID NO: 851) |
| h133-139_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRKNGTFLSLRDVFGKDLIYTLYYWKSSSSGKKTAKTNTN |
| | EFLIDVDKGENYCFSVQAVIPSRTVNRKSTDSPVECMGQEKGEF |
| | RE (SEQ ID NO: 852) |
| h140-145_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLTLRQVFGKDLIYTLYYWKSSSSGKKTAKTNTN |
| | EFLIDVDKGENYCFSVQAVIPSRTVNRKSTDSPVECMGQEKGEF |
| | RE (SEQ ID NO: 853) |
| h146-158_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLSLRDVFGNDLGYILTYRKSSSSGKKTAKTNTNE |
| | FLIDVDKGENYCFSVQAVIPSRTVNRKSTDSPVECMGQEKGEFR |
| | E (SEQ ID NO: 854) |
| h146-151_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLSLRDVFGNDLIYTLYYWKSSSSGKKTAKTNTN |
| | EFLIDVDKGENYCFSVQAVIPSRTVNRKSTDSPVECMGQEKGEF |
| | RE (SEQ ID NO: 855) |
| h152-158_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLSLRDVFGKDLGYILTYRKSSSSGKKTAKTNTNE |
| | FLIDVDKGENYCFSVQAVIPSRTVNRKSTDSPVECMGQEKGEFR |
| | E (SEQ ID NO: 856) |
| h159-219_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLSLRDVFGKDLIYTLYYWKDSSTGRKTNTTHTN |
| | EFLIDVEKGVSYCFFAQAVIFSRKTNHKSPESITKCTEQWKSVLG |
| | E (SEQ ID NO: 857) |
| h159-189_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLSLRDVFGKDLIYTLYYWKDSSTGRKTNTTHTN |
| | EFLIDVEKGVSYCFFAQAVIPSRTVNRKSTDSPVECMGQEKGEF |
| | RE (SEQ ID NO: 858) |
| h159-174_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLSLRDVFGKDLIYTLYYWKDSSTGRKTNTTHTN |
| | EFLIDVDKGENYCFSVQAVIPSRTVNRKSTDSPVECMGQEKGEF |
| | RE (SEQ ID NO: 859) |
| h159-166_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLSLRDVFGKDLIYTLYYWKDSSTGRKTAKTNTN |
| | EFLIDVDKGENYCFSVQAVIPSRTVNRKSTDSPVECMGQEKGEF |
| | RE (SEQ ID NO: 860) |
| h167-174_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLSLRDVFGKDLIYTLYYWKSSSSGKKTNTTHTN |
| | EFLIDVDKGENYCFSVQAVIPSRTVNRKSTDSPVECMGQEKGEF |
| | RE (SEQ ID NO: 861) |
| h175-189_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLSLRDVFGKDLIYTLYYWKSSSSGKKTAKTNTN |
| | EFLIDVEKGVSYCFFAQAVIPSRTVNRKSTDSPVECMGQEKGEF |
| | RE (SEQ ID NO: 862) |
| h190-219_r | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLSLRDVFGKDLIYTLYYWKSSSSGKKTAKTNTN |
| | EFLIDVDKGENYCFSVQAVIFSRKTNHKSPESITKCTEQWKSVL |
| | GE (SEQ ID NO: 863) |
| hTF_K68N | SGTTNTVAAYNLTWKSTNFKTILEWEPKPVNQVYTVQISTKSG |
| | DWKSKCFYTTDTECDLTDEIVKDVNQTYLARVFSYPAGNVEST |
| | GSAGEPLYENSPEFTPYLETNLGQPTIQSFEQVGTKVNVTVEDE |
| | RTLVRRNNTFLSLRDVFGKDLIYTLYYWKSSSSGKKTAKTNTN |
| | EFLIDVDKGENYCFSVQAVIPSRTVNRKSTDSPVECMGQEKGEF |
| | RE (SEQ ID NO: 865) |
| hTF_K149N | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLSLRDVFGNDLIYTLYYWKSSSSGKKTAKTNTN |
| | EFLIDVDKGENYCFSVQAVIPSRTVNRKSTDSPVECMGQEKGEF |
| | RE (SEQ ID NO: 866) |
| hTF_N171H_T197K | SGTTNTVAAYNLTWKSTNFKTILEWEPKPVNQVYTVQISTKSG |
| | DWKSKCFYTTDTECDLTDEIVKDVKQTYLARVFSYPAGNVEST |
| | GSAGEPLYENSPEFTPYLETNLGQPTIQSFEQVGTKVNVTVEDE |
| | RTLVRRNNTFLSLRDVFGKDLIYTLYYWKSSSSGKKTAKTHTN |
| | EFLIDVDKGENYCFSVQAVIPSRKVNRKSTDSPVECMGQEKGEF |
| | RE (SEQ ID NO: 867) |
| r141-194_h | AGTPPGKAFNLTWISTDFKTILEWQPKPTNYTYTVQISDRSRNW |
| | KYKCTGTTDTECDLTDEIVKDVNWTYEARVLSVPWRNSTHGK |
| | ETLFGTHGEEPPFTNARKFLPYRDTKIGQPVIQKYEQGGTKLKV |
| | TVKDSFTLVRRNNTFLSLRDVFGKDLIYTLYYWKSSSSGKKTA |
| | KTNTNEFLIDVDKGENYCFSVQAVIFSRKTNHKSPESITKCTEQ |
| | WKSVLGE (SEQ ID NO: 864) |

TABLE 57
Variable region sequence consensus

| Group | VH Domain Consensus (SEQ ID NO) | VL Domain Consensus (SEQ ID NO) |
|---|---|---|
| Lineage | QVQLVQSGAEVKKPGASVKVSCKAS | DIQMTQSPSTLSASVGDRVTITCx[R/Q] |
| 25A | GYTFDx[V/A]YGISWVRQAPGQGLEW | ASx[Q/E]SIx[S/N]x[S/N]WLAWYQQKP |
| | MGWIAPYx[N/S]GNTNYAQKLQGRVT | GKAPKLLIYKAx[S/Y]x[S/N]LEx[S/Y]G |
| | MTTDTSTSTAYMELRSLRSDDTAVYY | VPSRFSGSGSGTEFTLTISSLQPDDFAT |
| | CARDAGTYSPFGYGMDVWGQGTTVT | YYCQx[Q/L]FQx[S/K]LPPFTFGGGTKV |
| | VSS (SEQ ID NO: 868) | EIK (SEQ ID NO: 869) |
| Lineage | QVQLVQSGAEVKKPGASVKVSCKAS | DIQMTQSPSTLSASVGDRVTITCx[R/Q] |
| 25G | GYTFRSYGISWVRQAPGQGLEWMGW | ASx[Q/H]SIx[S/D]SWLAWYQQKPGKA |
| | VAPYx[N/S]GNTNYAQKLQGRVTMTT | PKLLIYx[K/S]ASx[S/Y]LESGVPSRFSG |
| | DTSTSTAYMELRSLRSDDTAVYYCAR | SGSGTEFTLTISSLQPDDFATYYCQx[Q/L/R] |
| | DAGTYSPYGYGMDVWGQGTTVTVSS | FQSLPPFTFGGGTKVEIK (SEQ ID |
| | (SEQ ID NO: 870) | NO: 871) |

TABLE 58
Consensus CDRs

| Antibody Group | | Lineage 25A | Lineage 25G |
|---|---|---|---|
| VH CDR | VH CDR1 | GYTFDx[V/A]YGIS | GYTFRSYGIS |
| Seq.* | | (SEQ ID NO: 872) | (SEQ ID NO: 878) |
| | VH CDR2 | WIAPYx[N/S]GNTNYAQKLQG | WVAPYx[N/S]GNTNYAQKLQG |
| | | (SEQ ID NO: 873) | (SEQ ID NO: 879) |
| | VH CDR3 | DAGTYSPFGYGMDV | DAGTYSPYGYGMDV |
| | | (SEQ ID NO: 874) | (SEQ ID NO: 880) |
| VL CDR | VL CDR1 | x[R/Q]ASx[Q/E]SIx[S/N]x | x[R/Q]ASx[Q/H]SIx[S/D]SWLA |
| Seq.* | | [S/N]WLA | (SEQ ID NO: 881) |
| | | (SEQ ID NO: 875) | |
| | VL CDR2 | KAx[S/Y]x[S/N]LEx[S/Y] | x[K/S]ASx[S/Y]LES |
| | | (SEQ ID NO: 876) | (SEQ ID NO: 882) |
| | VL CDR3 | Qx[Q/L]FQx[S/K]LPPFT | Qx[Q/L/R]FQSLPPFT |
| | | (SEQ ID NO: 877) | (SEQ ID NO: 883) |

*Exemplary CDR sequences encompass amino acids as determined by Kabat plus Chothia

TABLE 59
Antibody sequences for TF antibodies
variable regions in bold; cysteines involved in drug conjugation underlined.
The clones in able 13 have the same heavy chain constant regions. The clones
in Table 13 have the same light chain constant regions.

| Clone | HEAVY CHAIN | LIGHT CHAIN |
|---|---|---|
| 25A | QVQLVQSGAEVKKPGASVKVSC | DIQMTQSPSTLSASVGDRVTITCR |
| | KASGYTFDVYGISWVRQAPGQG | ASQSISSWLAWYQQKPGKAPKLL |
| | LEWMGWIAPYSGNTNYAQKLQ | IYKASSLESGVPSRFSGSGSGTEFT |
| | GRVTMTTDTSTSTAYMELRSLRS | LTISSLQPDDFATYYCQQFQSLPP |
| | DDTAVYYCARDAGTYSPFGYGM | FTFGGGTKVEIKRTVAAPSVFIFPP |
| | DVWGQGTTVTVSSASTKGPSVFP | SDEQLKSGTASVVCLLNNFYPREA |
| | LAPSSKSTSGGTAALGCLVKDYFP | KVQWKVDNALQSGNSQESVTEQD |
| | EPVTVSWNSGALTSGVHTFPAVLQ | SKDSTYSLSSTLTLSKADYEKHKVY |
| | SSGLYSLSSVVTVPSSSLGTQTYIC | ACEVTHQGLSSPVTKSFNRGEC |
| | NVNHKPSNTKVDKRVEPKSCDKT | (SEQ ID NO: 944) |
| | HTCPPCPAPELLGGPSVFLFPPKPK | |
| | DTLMISRTPEVTCVVVDVSHEDPE | |
| | VKFNWYVDGVEVHNAKTKPREE | |
| | QYNSTYRVVSVLTVLHQDWLNGK | |
| | EYKCKVSNKALPAPIEKTISKAKG | |
| | QPREPQVYTLPPSREEMTKNQVSL | |
| | TCLVKGFYPSDIAVEWESNGQPEN | |
| | NYKTTPPVLDSDGSFFLYSKLTVD | |
| | KSRWQQGNVFSCSVMHEALHNH | |
| | YTQKSLSLSPG (SEQ ID NO: 939) | |
| 25A3 | QVQLVQSGAEVKKPGASVKVSC | DIQMTQSPSTLSASVGDRVTITCQ |
| | KASGYTFDVYGISWVRQAPGQG | ASQSINNWLAWYQQKPGKAPKL |
| | LEWMGWIAPYSGNTNYAQKLQ | LIYKAYNLESGVPSRFSGSGSGTE |
| | GRVTMTTDTSTSTAYMELRSLRS | FTLTISSLQPDDFATYYCQLFQSL |
| | DDTAVYYCARDAGTYSPFGYGM | PPFTFGGGTKVEIKRTVAAPSVFIF |
| | DVWGQGTTVTVSSASTKGPSVFP | PPSDEQLKSGTASVVCLLNNFYPRE |
| | LAPSSKSTSGGTAALGCLVKDYFP | AKVQWKVDNALQSGNSQESVTEQ |
| | EPVTVSWNSGALTSGVHTFPAVLQ | DSKDSTYSLSSTLTLSKADYEKHKV |
| | SSGLYSLSSVVTVPSSSLGTQTYIC | YACEVTHQGLSSPVTKSFNRGEC |
| | NVNHKPSNTKVDKRVEPKSCDKT | (SEQ ID NO: 945) |
| | HTCPPCPAPELLGGPSVFLFPPKPK | |
| | DTLMISRTPEVTCVVVDVSHEDPE | |
| | VKFNWYVDGVEVHNAKTKPREE | |
| | QYNSTYRVVSVLTVLHQDWLNGK | |
| | EYKCKVSNKALPAPIEKTISKAKG | |
| | QPREPQVYTLPPSREEMTKNQVSL | |
| | TCLVKGFYPSDIAVEWESNGQPEN | |
| | NYKTTPPVLDSDGSFFLYSKLTVD | |
| | KSRWQQGNVFSCSVMHEALHNH | |
| | YTQKSLSLSPG (SEQ ID NO: 940) | |
| 25A5 | QVQLVQSGAEVKKPGASVKVSC | DIQMTQSPSTLSASVGDRVTITCR |
| | KASGYTFDVYGISWVRQAPGQG | ASESISNWLAWYQQKPGKAPKLL |
| | LEWMGWIAPYSGNTNYAQKLQ | IYKAYSLEYGVPSRFSGSGSGTEF |
| | GRVTMTTDTSTSTAYMELRSLRS | TLTISSLQPDDFATYYCQQFQKLP |
| | DDTAVYYCARDAGTYSPFGYGM | PFTFGGGTKVEIKRTVAAPSVFIFP |
| | DVWGQGTTVTVSSASTKGPSVFP | PSDEQLKSGTASVVCLLNNFYPREA |
| | LAPSSKSTSGGTAALGCLVKDYFP | KVQWKVDNALQSGNSQESVTEQD |
| | EPVTVSWNSGALTSGVHTFPAVLQ | SKDSTYSLSSTLTLSKADYEKHKVY |
| | SSGLYSLSSVVTVPSSSLGTQTYIC | ACEVTHQGLSSPVTKSFNRGEC |
| | NVNHKPSNTKVDKRVEPKSCDKT | (SEQ ID NO: 921) |
| | HTCPPCPAPELLGGPSVFLFPPKPK | |
| | DTLMISRTPEVTCVVVDVSHEDPE | |
| | VKFNWYVDGVEVHNAKTKPREE | |
| | QYNSTYRVVSVLTVLHQDWLNGK | |
| | EYKCKVSNKALPAPIEKTISKAKG | |
| | QPREPQVYTLPPSREEMTKNQVSL | |
| | TCLVKGFYPSDIAVEWESNGQPEN | |
| | NYKTTPPVLDSDGSFFLYSKLTVD | |
| | KSRWQQGNVFSCSVMHEALHNH | |
| | YTQKSLSLSPG (SEQ ID NO: 941) | |
| 25A5T | QVQLVQSGAEVKKPGASVKVSC | DIQMTQSPSTLSASVGDRVTITCR |
| | KASGYTFDAYGISWVRQAPGQG | ASESISNWLAWYQQKPGKAPKLL |
| | LEWMGWIAPYSGNTNYAQKLQ | IYKAYSLEYGVPSRFSGSGSGTEF |
| | GRVTMTTDTSTSTAYMELRSLRS | TLTISSLQPDDFATYYCQQFQKLP |
| | DDTAVYYCARDAGTYSPFGYGM | PFTFGGGTKVEIKRTVAAPSVFIFP |
| | DVWGQGTTVTVSSASTKGPSVFP | PSDEQLKSGTASVVCLLNNFYPREA |
| | LAPSSKSTSGGTAALGCLVKDYFP | KVQWKVDNALQSGNSQESVTEQD |
| | EPVTVSWNSGALTSGVHTFPAVLQ | SKDSTYSLSSTLTLSKADYEKHKVY |
| | SSGLYSLSSVVTVPSSSLGTQTYIC | ACEVTHQGLSSPVTKSFNRGEC |
| | NVNHKPSNTKVDKRVEPKSCDKT | (SEQ ID NO: 921) |
| | HTCPPCPAPELLGGPSVFLFPPKPK | |
| | DTLMISRTPEVTCVVVDVSHEDPE | |
| | VKFNWYVDGVEVHNAKTKPREE | |
| | QYNSTYRVVSVLTVLHQDWLNGK | |
| | EYKCKVSNKALPAPIEKTISKAKG | |
| | QPREPQVYTLPPSREEMTKNQVSL | |
| | TCLVKGFYPSDIAVEWESNGQPEN | |
| | NYKTTPPVLDSDGSFFLYSKLTVD | |
| | KSRWQQGNVFSCSVMHEALHNH | |
| | YTQKSLSLSPG (SEQ ID NO: 920) | |
| 25G | QVQLVQSGAEVKKPGASVKVSC | DIQMTQSPSTLSASVGDRVTITCR |
| | KASGYTFRSYGISWVRQAPGQG | ASQSISSWLAWYQQKPGKAPKLL |
| | LEWMGWVAPYNGNTNYAQKLQ | IYKASSLESGVPSRFSGSGSGTEFT |
| | GRVTMTTDTSTSTAYMELRSLRS | LTISSLQPDDFATYYCQQFQSLPP |
| | DDTAVYYCARDAGTYSPYGYGM | FTFGGGTKVEIKRTVAAPSVFIFPP |
| | DVWGQGTTVTVSSASTKGPSVFP | SDEQLKSGTASVVCLLNNFYPREA |
| | LAPSSKSTSGGTAALGCLVKDYFP | KVQWKVDNALQSGNSQESVTEQD |
| | EPVTVSWNSGALTSGVHTFPAVLQ | SKDSTYSLSSTLTLSKADYEKHKVY |
| | SSGLYSLSSVVTVPSSSLGTQTYIC | ACEVTHQGLSSPVTKSFNRGEC |
| | NVNHKPSNTKVDKRVEPKSCDKT | (SEQ ID NO: 946) |
| | HTCPPCPAPELLGGPSVFLFPPKPK | |
| | DTLMISRTPEVTCVVVDVSHEDPE | |
| | VKFNWYVDGVEVHNAKTKPREE | |
| | QYNSTYRVVSVLTVLHQDWLNGK | |
| | EYKCKVSNKALPAPIEKTISKAKG | |
| | QPREPQVYTLPPSREEMTKNQVSL | |
| | TCLVKGFYPSDIAVEWESNGQPEN | |
| | NYKTTPPVLDSDGSFFLYSKLTVD | |
| | KSRWQQGNVFSCSVMHEALHNH | |
| | YTQKSLSLSPG (SEQ ID NO: 942) | |
| 25G1 | QVQLVQSGAEVKKPGASVKVSC | DIQMTQSPSTLSASVGDRVTITCR |
| | KASGYTFRSYGISWVRQAPGQG | ASHSIDSWLAWYQQKPGKAPKLL |
| | LEWMGWVAPYSGNTNYAQKLQ | IYKASYLESGVPSRFSGSGSGTEF |
| | GRVTMTTDTSTSTAYMELRSLRS | TLTISSLQPDDFATYYCQLFQSLP |
| | DDTAVYYCARDAGTYSPYGYGM | PFTFGGGTKVEIKRTVAAPSVFIFP |
| | DVWGQGTTVTVSSASTKGPSVFP | PSDEQLKSGTASVVCLLNNFYPREA |
| | LAPSSKSTSGGTAALGCLVKDYFP | KVQWKVDNALQSGNSQESVTEQD |
| | EPVTVSWNSGALTSGVHTFPAVLQ | SKDSTYSLSSTLTLSKADYEKHKVY |
| | SSGLYSLSSVVTVPSSSLGTQTYIC | ACEVTHQGLSSPVTKSFNRGEC |
| | NVNHKPSNTKVDKRVEPKSCDKT | (SEQ ID NO: 947) |
| | HTCPPCPAPELLGGPSVFLFPPKPK | |
| | DTLMISRTPEVTCVVVDVSHEDPE | |
| | VKFNWYVDGVEVHNAKTKPREE | |
| | QYNSTYRVVSVLTVLHQDWLNGK | |
| | EYKCKVSNKALPAPIEKTISKAKG | |
| | QPREPQVYTLPPSREEMTKNQVSL | |
| | TCLVKGFYPSDIAVEWESNGQPEN | |
| | NYKTTPPVLDSDGSFFLYSKLTVD | |
| | KSRWQQGNVFSCSVMHEALHNH | |
| | YTQKSLSLSPG (SEQ ID NO: 922) | |
| 25G9 | QVQLVQSGAEVKKPGASVKVSC | DIQMTQSPSTLSASVGDRVTITCQ |
| | KASGYTFRSYGISWVRQAPGQG | ASQSIDSWLAWYQQKPGKAPKLL |
| | LEWMGWVAPYSGNTNYAQKLQ | IYSASYLESGVPSRFSGSGSGTEFT |
| | GRVTMTTDTSTSTAYMELRSLRS | LTISSLQPDDFATYYCQRFQSLPP |
| | DDTAVYYCARDAGTYSPYGYGM | FTFGGGTKVEIKRTVAAPSVFIFPP |
| | DVWGQGTTVTVSSASTKGPSVFP | SDEQLKSGTASVVCLLNNFYPREA |
| | LAPSSKSTSGGTAALGCLVKDYFP | KVQWKVDNALQSGNSQESVTEQD |
| | EPVTVSWNSGALTSGVHTFPAVLQ | SKDSTYSLSSTLTLSKADYEKHKVY |
| | SSGLYSLSSVVTVPSSSLGTQTYIC | ACEVTHQGLSSPVTKSFNRGEC |
| | NVNHKPSNTKVDKRVEPKSCDKT | (SEQ ID NO: 923) |
| | HTCPPCPAPELLGGPSVFLFPPKPK | |
| | DTLMISRTPEVTCVVVDVSHEDPE | |
| | VKFNWYVDGVEVHNAKTKPREE | |
| | QYNSTYRVVSVLTVLHQDWLNGK | |
| | EYKCKVSNKALPAPIEKTISKAKG | |
| | QPREPQVYTLPPSREEMTKNQVSL | |
| | TCLVKGFYPSDIAVEWESNGQPEN | |
| | NYKTTPPVLDSDGSFFLYSKLTVD | |
| | KSRWQQGNVFSCSVMHEALHNH | |
| | YTQKSLSLSPG (SEQ ID NO: 922) | |
| Lineage | QVQLVQSGAEVKKPGASVKVSC | DIQMTQSPSTLSASVGDRVTITCx[ |
| 25A | KASGYTFDx[V/A]YGISWVRQAP | R/QJASx[Q/E]SIx[S/N]x[S/N]WLAW |
| consensus | GQGLEWMGWIAPYx[N/S]GNTN | YQQKPGKAPKLLIYKAx[S/Y]x[S/ |
| | YAQKLQGRVTMTTDTSTSTAYM | NJLEx[S/Y]GVPSRFSGSGSGTEFT |
| | ELRSLRSDDTAVYYCARDAGTYS | LTISSLQPDDFATYYCQx[Q/L]FQx |
| | PFGYGMDVWGQGTTVTVSSAST | [S/K]LPPFTFGGGTKVEIKRTVAA |
| | KGPSVFPLAPSSKSTSGGTAALGCL | PSVFIFPPSDEQLKSGTASVVCLLNN |
| | VKDYFPEPVTVSWNSGALTSGVH | FYPREAKVQWKVDNALQSGNSQES |
| | TFPAVLQSSGLYSLSSVVTVPSSSL | VTEQDSKDSTYSLSSTLTLSKADYE |
| | GTQTYICNVNHKPSNTKVDKRVEP | KHKVYACEVTHQGLSSPVTKSFNR |
| | KSCDKTHTCPPCPAPELLGGPSVFL | GEC (SEQ ID NO: 948) |
| | FPPKPKDTLMISRTPEVTCVVVDV | |
| | SHEDPEVKFNWYVDGVEVHNAKT | |
| | KPREEQYNSTYRVVSVLTVLHQD | |
| | WLNGKEYKCKVSNKALPAPIEKTI | |
| | SKAKGQPREPQVYTLPPSREEMTK | |
| | NQVSLTCLVKGFYPSDIAVEWESN | |
| | GQPENNYKTTPPVLDSDGSFFLYS | |
| | KLTVDKSRWQQGNVFSCSVMHEA | |
| | LHNHYTQKSLSLSPG (SEQ ID NO: | |
| | 943) | |
| 43D7 | QVQLQQWGAGLLKPSETLSLTC | EIVLTQSPGTLSLSPGERATLSCR |
| | AVYGGSLSGYYWSWIRQPPGKG | ASDSVDSSYLAWYQQKPGQAPRL |
| | LEWIGEIGASGSTRYNPSLKSRV | LIYGAFSRANGIPDRFSGSGSGTD |
| | TISVDTSKNQFSLKLSSVTAADTA | FTLTISRLEPEDFAVYYCQQAGVV |
| | VYYCARDTPYYYEGGYYYYMD | PYTFGGGTKVEIKRTVAAPSVFIFP |
| | VWGKGTTVTVSSASTKGPSVFPL | PSDEQLKSGTASVVCLLNNFYPREA |
| | APSSKSTSGGTAALGCLVKDYFPE | KVQWKVDNALQSGNSQESVTEQD |
| | PVTVSWNSGALTSGVHTFPAVLQS | SKDSTYSLSSTLTLSKADYEKHKVY |
| | SGLYSLSSVVTVPSSSLGTQTYICN | ACEVTHQGLSSPVTKSFNRGEC |
| | VNHKPSNTKVDKRVEPKSCDKTH | (SEQ ID NO: 925) |
| | TCPPCPAPELLGGPSVFLFPPKPKD | |
| | TLMISRTPEVTCVVVDVSHEDPEV | |
| | KFNWYVDGVEVHNAKTKPREEQ | |
| | YNSTYRVVSVLTVLHQDWLNGKE | |
| | YKCKVSNKALPAPIEKTISKAKGQ | |
| | PREPQVYTLPPSREEMTKNQVSLT | |
| | CLVKGFYPSDIAVEWESNGQPENN | |
| | YKTTPPVLDSDGSFFLYSKLTVDK | |
| | SRWQQGNVFSCSVMHEALHNHYT | |
| | QKSLSLSPG (SEQ ID NO: 924) | |
| 43D8 | QVQLQQWGAGLLKPSETLSLTC | EIVLTQSPGTLSLSPGERATLSCR |
| | AVYGGSLSGYYWSWIRQPPGKG | ASQSVSSSFLAWYQQKPGQAPRL |
| | LEWIGEIGASGSTRYNPSLKSRV | LIYGAYSRATGIPDRFSGSGSGTD |
| | TISVDTSKNQFSLKLSSVTAADTA | FTLTISRLEPEDFAVYYCQQAGVV |
| | VYYCARDTPYYYEGGYYYYMD | PYTFGGGTKVEIKRTVAAPSVFIFP |
| | VWGKGTTVTVSSASTKGPSVFPL | PSDEQLKSGTASVVCLLNNFYPREA |
| | APSSKSTSGGTAALGCLVKDYFPE | KVQWKVDNALQSGNSQESVTEQD |
| | PVTVSWNSGALTSGVHTFPAVLQS | SKDSTYSLSSTLTLSKADYEKHKVY |
| | SGLYSLSSVVTVPSSSLGTQTYICN | ACEVTHQGLSSPVTKSFNRGEC |
| | VNHKPSNTKVDKRVEPKSCDKTH | (SEQ ID NO: 927) |
| | TCPPCPAPELLGGPSVFLFPPKPKD | |
| | TLMISRTPEVTCVVVDVSHEDPEV | |
| | KFNWYVDGVEVHNAKTKPREEQ | |
| | YNSTYRVVSVLTVLHQDWLNGKE | |
| | YKCKVSNKALPAPIEKTISKAKGQ | |
| | PREPQVYTLPPSREEMTKNQVSLT | |
| | CLVKGFYPSDIAVEWESNGQPENN | |
| | YKTTPPVLDSDGSFFLYSKLTVDK | |
| | SRWQQGNVFSCSVMHEALHNHYT | |
| | QKSLSLSPG (SEQ ID NO: 926) | |

1. A method of treating an inflammatory disease in a subject in need thereof comprising administering to the subject an isolated antibody wherein the antibody binds to Tissue Factor (TF), wherein the antibody binds human TF at a human TF binding site that is distinct from a human TF binding site bound by human FVIIa.

2. The method of claim 1, wherein the inflammatory disease is selected from the group consisting of colitis, inflammatory bowel disease, arthritis, acute lung injury, acute respiratory distress syndrome (ARDS), Respiratory Syncytial Virus (RSV), lupus, dermatitis, and dermatosis due to acute inflammatory component.

3. The method of claim 1, wherein the inflammatory disease is:
(i) a cardiovascular disease or injury;
(ii) a cardiovascular disease associated with upregulation of protease-activated receptor 2 (PAR-2);
(iii) a viral infection;
(iv) a neurological disease;
(v) rheumatism;
(vi) vasculitis;
(vii) an inflammatory skin disease;
(viii) an inflammatory disease that damages the skin; or
(ix) an inflammatory disease that damages the gastrointestinal tract.

4-10. (canceled)

11. The method of claim 3(i), wherein the cardiovascular disease or injury is myocardial infarction.

12. The method of claim 3(iii), wherein the viral infection is severe acute respiratory syndrome coronavirus 2 (SARS-CoV-2).

13. The method of claim 1, wherein:
(a) the antibody does not inhibit human thrombin generation as determined by thrombin generation assay (TGA); and/or
(b) the isolated human antibody does not inhibit or inhibits human thrombin generation to a lesser extent, as determined by thrombin generation assay (TGA), compared to a reference antibody comprising a heavy chain variable domain (VH) sequence of SEQ ID NO:821 and a light chain variable domain (VL) sequence of SEQ ID NO:822, optionally wherein binding between the isolated antibody and a variant TF extracellular domain comprising a mutation at amino acid residue 149 of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; and/or
(c) the antibody comprises:
(i) all three heavy chain Complementary Determining Regions (CDRs) and all three light chain CDRs from an antibody group in Table 35 wherein the all three heavy chain CDRs and the all three light chain CDRs are from the same antibody group, or
(ii) all three heavy chain CDRs and all three light chain CDRs from an antibody in any one of Tables 15-34, wherein the all three heavy chain CDRs and the all three light chain CDRs are from the same antibody, optionally comprising all three heavy chain CDRs and all three light chain CDRs from: the antibody designated 25A, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea; or
(iii) a VH sequence and VL sequence from Table 14, wherein the VH and VL sequences are from the same group in Table 14; or
(iv) a VH sequence and VL sequence from Table 13, wherein the VH and VL sequences are from the same clone in Table 13.

14-22. (canceled)

23. The method of claim 1, wherein the antibody comprises three heavy chain complementarity determining regions (CDRs) (VH-CDR1, VH-CDR2, and VH-CDR3) and three light chain complementarity determining regions (CDRs) (VL-CDR1, VL-CDR2, and VL-CDR3), wherein: the VH-CDR1 comprises the amino acid sequence set forth in SEQ ID NO:797; the VH-CDR2 comprises the amino acid sequence set forth in SEQ ID NO:798; the VH-CDR3 comprises the amino acid sequence set forth in SEQ ID NO:799; the VL-CDR1 comprises the amino acid sequence set forth in SEQ ID NO:800; the VL-CDR2 comprises the amino acid sequence set forth in SEQ ID NO:801; and the VL-CDR3 comprises the amino acid sequence set forth in SEQ ID NO:802, optionally wherein
the VH-CDR1 comprises the amino acid sequence set forth in SEQ ID NO:571; the VH-CDR2 comprises the amino acid sequence set forth in SEQ ID NO:572; the VH-CDR3 comprises the amino acid sequence set forth in SEQ ID NO:573; the VL-CDR1 comprises the amino acid sequence set forth in SEQ ID NO:574; the VL-CDR2 comprises the amino acid sequence set forth in SEQ ID NO:575; and the VL-CDR3 comprises the amino acid sequence set forth in SEQ ID NO:576; or
the VH-CDR1 comprises the amino acid sequence set forth in SEQ ID NO:609; the VH-CDR2 comprises the amino acid sequence set forth in SEQ ID NO:610; the VH-CDR3 comprises the amino acid sequence set forth in SEQ ID NO:611; the VL-CDR1 comprises the amino acid sequence set forth in SEQ ID NO:612, the VL-CDR2 comprises the amino acid sequence set forth in SEQ ID NO:613; and the VL-CDR3 comprises the amino acid sequence set forth in SEQ ID NO:614; or
the antibody comprises: a heavy chain variable domain (VH) sequence comprising the amino acid sequence set forth in SEQ ID NO:769 and a light chain variable domain (VL) sequence comprising the amino acid sequence set forth in SEQ ID NO:770, optionally wherein the antibody comprises: (i) a VH sequence comprising the amino acid sequence set forth in SEQ ID NO:569 and a VL sequence comprising the amino acid sequence set forth in SEQ ID NO:570, or (ii) a VH sequence comprising the amino acid sequence set forth in SEQ ID NO:607 and a VL sequence comprising the amino acid sequence set forth in SEQ ID NO:608; or
the antibody comprises: a heavy chain comprising the amino acid sequence set forth in SEQ ID NO:924 and a light chain comprising the amino acid sequence set forth in SEQ ID NO:925; or
the antibody comprises: a VH sequence comprising the amino acid sequence set forth in SEQ ID NO:645 and a VL sequence comprising the amino acid sequence set forth in SEQ ID NO:646; or
the antibody comprises: a heavy chain comprising the amino acid sequence set forth in SEQ ID NO:926 and a light chain comprising the amino acid sequence set forth in SEQ ID NO:927.

24-31. (canceled)

32. The method of claim 1, wherein the antibody comprises three heavy chain complementarity determining regions (CDRs) (VH-CDR1, VH-CDR2, and VH-CDR3) and three light chain complementarity determining regions (CDRs) (VL-CDR1, VL-CDR2, and VL-CDR3), wherein: the VH-CDR1 comprises the amino acid sequence set forth in SEQ ID NO:779; the VH-CDR2 comprises the amino acid sequence set forth in SEQ ID NO:780; the VH-CDR3 comprises the amino acid sequence set forth in SEQ ID NO:781; the VL-CDR1 comprises the amino acid sequence set forth in SEQ ID NO:782; the VL-CDR2 comprises the amino acid sequence set forth in SEQ ID NO:783; and the VL-CDR3 comprises the amino acid sequence set forth in SEQ ID NO:784; or
the VH-CDR1 comprises the amino acid sequence set forth in SEQ ID NO:872; the VH-CDR2 comprises the amino acid sequence set forth in SEQ ID NO:873; the VH-CDR3 comprises the amino acid sequence set forth in SEQ ID NO:874; the VL-CDR1 comprises the amino acid sequence set forth in SEQ ID NO:875; the VL-CDR2 comprises the amino acid sequence set forth in SEQ ID NO:876; and the VL-CDR3 comprises the amino acid sequence set forth in SEQ ID NO:877, optionally wherein the VH-CDR1 comprises the amino acid sequence set forth in SEQ ID NO:884; the VH-CDR2 comprises the amino acid sequence set forth in SEQ ID NO:885; the VH-CDR3 comprises the amino acid sequence set forth in SEQ ID NO:886; the VL-CDR1 comprises the amino acid sequence set forth in SEQ ID NO:887; the VL-CDR2 comprises the amino acid sequence set forth in SEQ ID NO:888; and the VL-CDR3 comprises the amino acid sequence set forth in SEQ ID NO:889; or
the antibody comprises: a heavy chain variable domain (VH) sequence comprising the amino acid sequence set forth in SEQ ID NO:868 and a light chain variable domain (VL) sequence comprising the amino acid sequence set forth in SEQ ID NO:869, optionally wherein the antibody comprises: (i) a VH sequence comprising the amino acid sequence set forth in SEQ ID NO:189 and a VL sequence comprising the amino acid sequence set forth in SEQ ID NO:190, or (ii) a VH sequence comprising the amino acid sequence set forth in SEQ ID NO:836 and a VL sequence comprising the amino acid sequence set forth in SEQ ID NO:837; or
the antibody comprises: a heavy chain comprising the amino acid sequence set forth in SEQ ID NO:920 and a light chain comprising the amino acid sequence set forth in SEQ ID NO:921.

33-38. (canceled)

39. The method of claim 1, wherein the antibody comprises three heavy chain complementarity determining regions (CDRs) (VH-CDR1, VH-CDR2, and VH-CDR3) and three light chain complementarity determining regions (CDRs) (VL-CDR1, VL-CDR2, and VL-CDR3), wherein: the VH-CDR1 comprises the amino acid sequence set forth in SEQ ID NO:878; the VH-CDR2 comprises the amino acid sequence set forth in SEQ ID NO:879; the VH-CDR3 comprises the amino acid sequence set forth in SEQ ID NO:880; the VL-CDR1 comprises the amino acid sequence set forth in SEQ ID NO:881; the VL-CDR2 comprises the amino acid sequence set forth in SEQ ID NO:882; and the VL-CDR3 comprises the amino acid sequence set forth in SEQ ID NO:883, optionally wherein the VH-CDR1 comprises the amino acid sequence set forth in SEQ ID NO:267; the VH-CDR2 comprises the amino acid sequence set forth in SEQ ID NO:268; the VH-CDR3 comprises the amino acid sequence set forth in SEQ ID NO:269; the VL-CDR1 comprises the amino acid sequence set forth in SEQ ID NO:270; the VL-CDR2 comprises the amino acid sequence set forth in SEQ ID NO:271; and the VL-CDR3 comprises the amino acid sequence set forth in SEQ ID NO:272; or
the antibody comprises: a heavy chain variable domain (VH) sequence comprising the amino acid sequence set forth in SEQ ID NO:870 and a light chain variable domain (VL) sequence comprising the amino acid sequence set forth in SEQ ID NO:871, optionally wherein the antibody comprises: a VH sequence comprising the amino acid sequence set forth in SEQ ID NO:303 and a VL sequence comprising the amino acid sequence set forth in SEQ ID NO:304; or
the antibody comprises: a heavy chain comprising the amino acid sequence set forth in SEQ ID NO:922 and a light chain comprising the amino acid sequence set forth in SEQ ID NO:923.

40-43. (canceled)

44. The method of claim 1, wherein:
the antibody competes for binding to human TF with the antibody designated 25A, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea; and/or
the antibody binds to the same human TF epitope bound by the antibody designated 25A, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea.

45-49. (canceled)

50. The method of claim 1, wherein the antibody does not inhibit human thrombin generation as determined by thrombin generation assay (TGA), does not reduce the thrombin peak on a thrombin generation curve (Peak IIa) compared to an isotype control, does not increase the time from the assay start to the thrombin peak on a thrombin generation curve (ttPeak) compared to an isotype control, does not decrease the endogenous thrombin potential (ETP) as determined by the area under a thrombin generation curve compared to an isotype control, allows human thrombin generation as determined by thrombin generation assay (TGA), maintains the thrombin peak on a thrombin generation curve (Peak IIa) compared to an isotype control, maintains the time from the assay start to the thrombin peak on a thrombin generation curve (ttPeak) compared to an isotype control, preserves the endogenous thrombin potential (ETP) as determined by the area under a thrombin generation curve compared to an isotype control, binds human TF at a human TF binding site that is distinct from a human TF binding site bound by human FX, does not interfere with the ability of TF:FVIIa to convert FX into FXa, and does not compete for binding to human TF with FVIIa.

51. The method of claim 1, wherein:
the three heavy chain CDRs and the three light chain CDRs are determined using exemplary, Kabat, Chothia, AbM, Contact, or IMGT numbering; and/or
the antibody specifically binds to cynomolgus TF; and/or
the antibody binds to specifically mouse TF; and/or
the antibody binds to specifically rabbit TF; and/or
the antibody binds to specifically pig TF; and/or
the disease involves vascular inflammation; and/or
the disease involves local inflammation; and/or
the disease involves systemic inflammation; and/or
the disease involves infiltration of mononuclear cells and/or granulocytes, optionally wherein the mononuclear cells comprise macrophages and/or lymphocytes, and/or
wherein the granulocytes comprise neutrophils and/or eosinophils.

52-62. (canceled)

63. The method of claim 1, wherein:
(a) upon administration to a subject, the antibody reduces the total leukocyte count, optionally wherein the total leukocyte count is determined by light microscopy; and/or
(b) upon administration to a subject, the antibody reduces the total number of granulocytes, optionally wherein the granulocytes comprise neutrophils and/or eosinophils, optionally wherein the total number of granulocytes is determined by immunohistochemical (IHC) analysis or bronco-alveolar lavage (BAL) fluid differential cell count, optionally wherein the granulocytes are in the alveoli and/or in the interstitial fluid; and/or
(c) upon administration to a subject, the antibody reduces the total number of mononuclear cells, optionally wherein the mononuclear cells comprise macrophages optionally M1 macrophages and/or wherein the mononuclear cells comprise lymphocytes and/or wherein the mononuclear cells comprise monocytes, optionally wherein the total number of mononuclear cells is determined by immunohistochemical (IHC) analysis or bronco-alveolar lavage (BAL) fluid differential cell count, optionally wherein the mononuclear cells are in the alveoli or in the interstitial fluid; and/or
(d) upon administration to a subject, the subject maintains or increases body weight relative to baseline levels; and/or
(e) upon administration to a subject, the antibody maintains or increases body weight relative to a different anti-inflammatory therapeutic; and/or
(f) upon administration to a subject, the antibody reduces the spleen size or reverses spleen enlargement relative to baseline levels, and/or wherein the antibody reduces the spleen size or reverses splenomegaly relative to a different anti-inflammatory therapeutic, optionally wherein the spleen size or splenomegaly is determined using palpation, percussion, ultrasound, computerized tomography (CT) scan or magnetic resonance imagining (MRI); and/or
(g) the inflammatory disease is acute lung injury or acute respiratory distress syndrome (ARDS); and/or
(h) upon administration to a subject, the antibody increases net alveolar fluid clearance relative to baseline levels or relative to a different anti-inflammatory therapeutic, optionally wherein net alveolar fluid clearance is determined by measuring sequential edema fluid protein concentrations, optionally wherein the sequential edema fluid protein concentrations are measured with ELISA; and/or
(i) the inflammatory disease is SARS-Cov-2 and wherein upon administration to a subject, the subject maintains or increases body weight relative to baseline levels, optionally wherein the antibody maintains or increases body weight relative to a different anti-inflammatory therapeutic; and/or
(j) upon administration to a subject, the antibody reduces the concentration of inflammatory cytokines and chemokines relative to baseline levels and/or reduces the concentration of inflammatory cytokines and chemokines relative to a different anti-inflammatory therapeutic, optionally wherein the inflammatory cytokines and chemokines are in bronco-alveolar lavage (BAL) samples and/or in lung homogenate samples, optionally wherein the inflammatory cytokines and chemokines comprise one or more of: IL-1α, IL-1β, IL-2, IL-4, IL-5, IL-6, IL-8, IL-10, IFNγ, GM-CSF, TNFα, CCL2, CCL3, CCL4, CCL5, CCL19, CCL20, CCL25, CXCL1, CXCL2, and CXCL10, or wherein the inflammatory cytokines and chemokines comprise VEGF, or wherein the inflammatory cytokines and chemokines comprise one or more of: GMCSF, VEGF, IL17F, IL-1 beta, IL-6, IFNγ, IL-8, and KC, optionally wherein the inflammatory cytokines and chemokines are measured using ELISA or using Luminex Multiplex Assay.

64-101. (canceled)

102. The method of claim 1, wherein the inflammatory disease is (i) a viral infection or (ii) RSV.

103. The method of claim 102, wherein in part (ii):
upon administration to a subject, the antibody increases anti-inflammatory cytokines and chemokines relative to baseline levels; and/or
upon administration to a subject, the antibody increases anti-inflammatory cytokines and chemokines relative to a different anti-inflammatory therapeutic; and/or
the anti-inflammatory cytokines and chemokines comprise one or more of: IL-10 and IL27p28; and/or
the anti-inflammatory cytokines and chemokines are in bronco-alveolar lavage (BAL) samples; and/or
the inflammatory cytokines and chemokines are measured using multiplex electrochemiluminescence MSD assay or using Luminex Multiplex Assay.

104-109. (canceled)

110. The method of claim 1, wherein upon administration to a subject, the antibody reduces fibrosis in the lungs relative to baseline levels and/or relative to a different anti-inflammatory therapeutic.

111. (canceled)

112. The method of claim 110, wherein the fibrosis is determined by IHC analysis or Quantitative High Resolution Computed Tomography (qHRCT).

113. (canceled)

114. The method of claim 1, wherein:
the inflammatory disease is arthritis, optionally wherein upon administration to a subject, the antibody reduces the concentration of inflammatory cytokines and chemokines relative to baseline levels and/or relative to a different anti-inflammatory therapeutic, optionally wherein the inflammatory cytokines and chemokines comprise one or more of: IL-1α, IL-1β, IL-2, IL-4, IL-5, IL-6, IL-8, IL-10, IFNγ, GM-CSF, TNFα, CCL2, CCL3, CCL4, CCL5 CCL19, CCL20, CCL25, CXCL1, CXCL2, and CXCL10;
or
the inflammatory disease is colitis or inflammatory bowel disease, optionally wherein upon administration to a subject, the antibody results in a normal stool consistency or hardens the subject's stool consistency relative to baseline levels and/or relative to a different anti-inflammatory therapeutic optionally wherein the stool consistency is determined using the Bristol Stool Scale; and/or wherein upon administration to a subject, the antibody reduces blood or results in the absence of blood in the subject's stool relative to baseline levels or relative to a different anti-inflammatory therapeutic, optionally wherein the blood in the subject's stool is measured using a hemoccult test; and/or wherein upon administration to a subject, the antibody reduces the concentration of inflammatory cytokines and chemokines relative to baseline levels or relative to a different anti-inflammatory therapeutic, optionally wherein the inflammatory cytokines and chemokines comprise one or more of: IL-1α, IL-1β, IL-2, IL-4, IL-5, IL-6, IL-8, IL-10, IFNγ, GM-CSF, TNFα, CCL2, CCL3, CCL4, CCL5, CCL19, CCL20, CCL25, CXCL1, CXCL2, and CXCL10;
or
the inflammatory disease is myocardial infarction, optionally wherein the antibody reduces infarct size relative to baseline levels and/or relative to a different anti-inflammatory therapeutic and/or wherein upon administration to a subject, the antibody increases left ventricular ejection fraction relative to baseline levels and/or relative to a different anti-inflammatory therapeutic, and/or wherein upon administration to a subject, the antibody decreases left ventricular end diastolic volume relative to baseline levels or relative to a different anti-inflammatory therapeutic, and/or wherein upon administration to a subject, the antibody decreases inflammatory cell recruitment in the infarcted myocardium relative to baseline levels or relative to a different anti-inflammatory therapeutic, optionally wherein the inflammatory cells are selected from CD45+, CD11b+, Ly6Chi, CD45+/CD90.2−/NK1.1−/CD11b+, CD45+/CD90.2−/NK1.1−/CD11b+/Ly6Chi, and CD45+/CD90.2−/NK1.1−/CD11b+/Ly6Clo, optionally wherein the inflammatory cell recruitment is measured using flow cytometry.

115-139. (canceled)

140. The method of claim 1, wherein:
upon administration to a subject, the antibody results in a reduced need for systemic steroids; and/or
wherein the different anti-inflammatory therapeutic comprises one or more of: a non-steroidal anti-inflammatory drug (NSAID), a steroidal anti-inflammatory drug, a beta-agonist, an anticholinergic agent, an antihistamine, and a methyl xanthine; and/or
the different anti-inflammatory therapeutic comprises any one of: an IL-6 inhibitor, anti-GM-CSF, anti-TNFa, anti-IL-1a, dexamethasone, a chemokine and chemokine receptor antagonist, and a JAK inhibitor.

141-142. (canceled)

143. The method of claim 1, wherein the antibody is administered biweekly or weekly.

144. (canceled)
